(19) United States
(12) Patent Application Publication
(10) Pub. No.: US 2004/0005557 A1
(43) Pub. Date: Jan. 8, 2004

(54) Proteins, polynucleotides encoding them and methods of using the same

(76) Inventors: Muralidhara Padigaru, Branford, CT (US); John P. Alsobrook II, Madison, CT (US); Steven D. Colman, Guilford, CT (US); Kimberly A. Spytek, New Haven, CT (US); Ferenc L. Boldog, North Haven, CT (US); Corine A.M. Vernet, Branford, CT (US); Li Li, Branford, CT (US); Suresh G. Shenoy, Branford, CT (US); Stacie J. Casman, North Haven, CT (US); Xiaojia (Sasha) Guo, Branford, CT (US); Shlomit R. Edinger, New Haven, CT (US); John R. MacDougall, Hamden, CT (US); Uriel M. Malyankar, Branford, CT (US); Meera Patturajan, Branford, CT (US); Richard A. Shimkets, Guilford, CT (US); Carol E. A. Pena, New Haven, CT (US); Velizar T. Tchernev, Branford, CT (US); Bryan D. Zerhusen, Branford, CT (US); Isabelle Millet, Milford, CT (US); Charles E. Miller, Guilford, CT (US); Denise M. Lepley, Branford, CT (US); Glennda Smithson, Guilford, CT (US); Jason C. Baumgartner, New Haven, CT (US); John L. Herrmann, Guilford, CT (US); John A. Peyman, New Haven, CT (US); Linda Gorman, Branford, CT (US); Peter D. Mezes, Old Lyme, CT (US); Ramesh Kekuda, Norwalk, CT (US); Raymond J. Taupier JR., East Haven, CT (US); Valerie Gerlach, Branford, CT (US); William M. Grosse, Branford, CT (US); Xiaohong Liu, Lexington, MA (US); Karen Ellerman, Branford, CT (US); Mark Rothenberg, Clinton, CT (US); David J. Stone, Guilford, CT (US); Catherine E. Burgess, Wethersfield, CT (US)

Correspondence Address: MINTZ, LEVIN, COHN, FERRIS,, GLOVSKY and POPEO, P.C., One Financial Center, Boston, MA, 02111, US

(21) Appl. No.: 10/051,874

(22) Filed: Jan. 16, 2002

Related U.S. Application Data
(60) Provisional application No. 60/261,376, filed on Jan. 16, 2001.
(60) Provisional application No. 60/268,595, filed on Feb. 14, 2001.
(60) Provisional application No. 60/325,306, filed on Sep. 27, 2001.
(60) Provisional application No. 60/262,587, filed on Jan. 18, 2001.
(60) Provisional application No. 60/272,409, filed on Feb. 28, 2001.
(60) Provisional application No. 60/262,454, filed on Jan. 18, 2001.
(60) Provisional application No. 60/276,777, filed on Mar. 16, 2001.
(60) Provisional application No. 60/291,672, filed on May 17, 2001.
(60) Provisional application No. 60/330,336, filed on Oct. 18, 2001.
(60) Provisional application No. 60/265,530, filed on Jan. 31, 2001.
(60) Provisional application No. 60/345,202, filed on Nov. 9, 2001.

(51) Int. Cl. C12Q 1/68; C07H 21/04; C12N 9/00; C12P 21/02; C12N 5/06; C07K 14/435

(52) U.S. Cl. 435/6; 435/69.1; 435/183; 435/320.1; 435/325; 530/350; 536/23.2

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This is a request for filing a new nonprovisional application under 37 C.F.R. §1.53(b). This application claims priority to U.S. Ser. No. 60/261,376, filed Jan. 16, 2001 (Cura-545); U.S. Ser. No. 60/268,595, filed Feb. 14, 2001 (Cura-545 C1); U.S. Ser. No. 60/325,306, filed Sep. 27, 2001 (Cura-545 I1); U.S. Ser. No. 60/262,587, filed Jan. 18, 2001 (Cura-549); U.S. Ser. No. 60/272,409, filed Feb. 28, 2001 (Cura-549 A); U.S. Ser. No. 60/______, filed Nov. 9, 2001 (Cura-549 B); U.S. Ser. No. 60/262,454, filed Jan. 18, 2001 (Cura-550); U.S. Ser. No. 60/276,777, filed Mar. 16, 2001 (Cura-550 B1); U.S. Ser. No. 60/291,672, filed May 17, 2001 (Cura-550 B2); U.S. Ser. No. 60/330,336, filed Oct. 18, 2001 (Cura-550 D1); U.S. Ser. No. 60/265,530, filed Jan. 31, 2001 (Cura-550 A), each of which is incorporated by reference in its entirety.

### FIELD OF THE INVENTION

The invention relates to polynucleotides and the polypeptides encoded by such polynucleotides, as well as vectors, host cells, antibodies and recombinant methods for producing the polypeptides and polynucleotides, as well as methods for using the same.

### BACKGROUND OF THE INVENTION

The present invention is based in part on nucleic acids encoding proteins that are new members of the following protein families: fibromodulin, secretin receptor precursor, B7-H2, B7-H1, prostasin, lysosomal acid lipase, tryptase 4, P450, mitsugumin29, micromolar calcium-activated neutral protease 1, P2X2C, DIABLO, HRPET-1 related protein, B7-H2B, galactosyltransferase, lymphocyte antigen precursor, pepsinogen C and ALR. More particularly, the invention relates to nucleic acids encoding novel polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9, NOV10, NOV11, NOV12, NOV13, NOV14, NOV15, NOV16, NOV17 and NOV18 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56. The nucleic acid can be, for example, a genomic DNA fragment or a cDNA molecule that includes the nucleic acid sequence of any of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55.

Also included in the invention is an oligonucleotide, e.g., an oligonucleotide which includes at least 6 contiguous nucleotides of a NOVX nucleic acid (e.g., SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55) or a complement of said oligonucleotide. Also included in the invention are substantially purified NOVX polypeptides (SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., trauma, regeneration (in vitro and in vivo), viral/bacterial/parasitic infections, Von Hippel-Lindau (VHL) syndrome, Alzheimer's disease, stroke, Tuberous sclerosis, hypercalceimia, Parkinson's disease, Huntington's disease, Cerebral palsy, Epilepsy, Lesch-Nyhan syndrome, multiple sclerosis, Ataxia-telangiectasia, leukodystrophies, behavioral disorders, addiction, anxiety, pain, actinic keratosis, acne, hair growth diseases, allopecia, pigmentation disorders, endocrine disorders, connective tissue disorders, such as severe neonatal Marfan syndrome, dominant ectopia lentis, familial ascending aortic aneurysm, isolated skeletal features of Marfan syndrome, Shprintzen-Goldberg syndrome, genodermatoses, contractural arachnodactyly, inflammatory disorders such as osteo- and rheumatoid-arthritis, inflammatory bowel disease, Crohn's disease; immunological disorders, AIDS; cancers including but not limited to lung cancer, colon cancer, neoplasm; adenocarcinoma; lymphoma; prostate cancer; uterus cancer, leukemia or pancreatic cancer; blood disorders; asthma; psoriasis; vascular disorders, hypertension, skin disorders, renal disorders including Alport syndrome, immunological disorders, tissue injury, fibrosis disorders, bone diseases, Ehlers-Danlos syndrome type VI, VII, type IV, S-linked cutis laxa and Ehlers-Danlos syndrome type V, osteogenesis imperfecta, neurologic diseases, brain and/or autoimmune disorders like encephalomyelitis, neurodegenerative disorders, immune disorders, hematopoietic disorders, muscle disorders, inflammation and wound repair, bacterial, fungal, protozoal and viral infections (particularly infections caused by HIV-1 or HIV-2), pain, acute heart failure, hypotension, hypertension, urinary retention, osteoporosis, treatment of Albright hereditary ostoeodystrophy, angina pectoris, myocardial infarction, ulcers, benign prostatic hypertrophy, arthrogryposis multiplex congenita, osteogenesis imperfecta, keratoconus, scoliosis, duodenal atresia, esophageal atresia, intestinal malrotation, pancreatitis, obesity systemic lupus erythematosus, autoimmune disease, emphysema, scleroderma, allergy, ARDS, neuroprotection, fertility Myasthenia gravis, diabetes, obesity, growth and reproductive disorders hemophilia, hypercoagulation, idiopathic thrombocytopenic purpura, immunodeficiencies, graft vesus host, adrenoleukodystrophy, congenital adrenal hyperplasia, endometriosis, xerostomia, ulcers, cirrhosis, transplantation, diverticular disease, Hirschsprung's disease, appendicitis, arthritis, ankylosing spondylitis, tendinitis, renal artery stenosis, interstitial nephritis, glomerulonephritis, polycystic kidney disease, erythematosus, renal tubular acidosis, IgA nephropathy, anorexia, bulimia, psychotic disorders, including anxiety, schizophrenia, manic depression, delirium, dementia, severe mental retardation and dyskinesias, such as Huntington's disease and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

NOVX nucleic acids and polypeptides are further useful in the generation of antibodies that bind immuno-specifically to the novel NOVX substances for use in therapeutic or diagnostic methods. These NOVX antibodies may be generated according to methods known in the art, using prediction from hydrophobicity charts, as described in the “Anti-NOVX Antibodies” section below. The disclosed NOVX proteins have multiple hydrophilic regions, each of which can be used as an immunogen. These NOVX proteins can be used in assay systems for functional analysis of various human disorders, which will help in understanding of pathology of the disease and development of new drug targets for various disorders.

The NOVX nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE 1
Sequences and Corresponding SEQ ID Numbers

| | | | Nucleic | Amino |
|---|---|---|---|---|
| | | | Acid | Acid |
| NOVX | Internal | | SEQ ID | SEQ ID |
| No. | Acc. No. | Homology | NO. | NO. |
| 1a | CG56201-01 | Fibromodulin | 1 | 2 |
| 1b | CG56201-02 | Fibromodulin | 3 | 4 |
| 1c | CG56201-04 | Fibromodulin | 5 | 6 |
| 1d | CG56201-01 | Fibromodulin | 7 | 8 |
| | Assembly | | | |
| 2a | CG56213-01 | Secretin receptor | 9 | 10 |
| | | precursor | | |
| 2b | CG56213-02 | Secretin receptor | 11 | 12 |
| | | precursor | | |
| 2c | CG56213-03 | Secretin receptor | 13 | 14 |
| | | precursor | | |
| 3a | CG55790-03 | B7-H2 | 15 | 16 |
| 3b | CG55790-04 | B7-H2 | 17 | 18 |
| 4a | CG56110-01 | B7-H1 | 19 | 20 |
| 4b | CG56110-04 | B7-H1 | 21 | 22 |
| 5a | CG56142-01 | Prostasin | 23 | 24 |
| 5b | CG56142-02 | Prostasin | 25 | 26 |
| 6a | CG50159-01 | Lysosomal acid lipase | 27 | 28 |
| 6b | CG50159-02 | Lysosomal acid lipase | 29 | 30 |
| 6c | CG50159-04 | Lysosomal acid lipase | 31 | 32 |
| 7 | CG56140-01 | Tryptase 4 | 33 | 34 |
| 8 | CG56134-01 | P450 | 35 | 36 |
| 9 | CG56207-01 | Mitsugumin29 | 37 | 38 |
| 10 | CG56127-01 | Micromolar calcium- | 39 | 40 |
| | | activated | | |
| | | neutral protease1 | | |
| 11 | CG56179-01 | P2X2C | 41 | 42 |
| 12 | CG56132-01 | DIABLO | 43 | 44 |
| 13 | CG56195-01 | HRPET-1 | 45 | 46 |
| 14 | CG55790-02 | B7-H2B | 47 | 48 |
| 15 | CG56252-01 | Galactosyltransferase | 49 | 50 |
| 16 | CG56303-01 | Lymphocyte antigen | 51 | 52 |
| | | precursor | | |
| 17 | CG56307-01 | Pepsinogen C | 53 | 54 |
| 18 | CG56294-01 | ALR | 55 | 56 |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1 is homologous to the Fibromodulin family of proteins. Thus, the NOV1 nucleic acids, polypeptides, antibodies and related compounds according to the invention will be useful in therapeutic and diagnostic applications implicated in, for example, the treatment of patients suffering from: repair of damage to cartilage and ligaments; therapeutic applications to joint repair, and other diseases, disorders and conditions of the like.

It has been suggested that fibromodulin participates in the assembly of the extracellular matrix by virtue of its ability to interact with type I and type II collagen fibrils and to inhibit fibrillogenesis in vitro.

NOV2 is homologous to the Secretin receptor precursor-like family of proteins. Thus NOV2 nucleic acids, polypeptides, antibodies and related compounds according to the invention will be useful in therapeutic and diagnostic applications implicated in, for example, developmental diseases, MHCII and III diseases (immune diseases), taste and scent detectability disorders, Burkitt's lymphoma, corticoneurogenic disease, signal transduction pathway disorders, retinal diseases including those involving photoreception, cell growth rate disorders; cell shape disorders, feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and treatment of Albright hereditary ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation, dentatorubro-pallidoluysian atrophy (DRPLA) hypophosphatemic rickets, autosomal dominant (2) acrocallosal syndrome and dyskinesias, such as Huntington's disease or Gilles de la Tourette syndrome and/or other pathologies and disorders of the like.

Secretin occupies a unique position in the history of gastrointestinal hormones because it was the first to be discovered, in duodenal mucosa by Bayliss and Starling (1902). This 27-amino acid peptide stimulates the secretion of bicarbonate, enzymes, and potassium ion by the pancreas.

NOV3 is homologous to the B7-H2 like proteins. Thus, the NOV3 nucleic acids and polypeptides, antibodies and related compounds according to the invention will be useful in therapeutic and diagnostic applications implicated in, for example, the treatment of patients suffering from brain disorders including epilepsy, eating disorders, schizophrenia, ADD, and cancer; heart disease; inflammation and autoimmune disorders including Crohn's disease, IBD, allergies, rheumatoid and osteoarthritis, inflammatory skin disorders, allergies, blood disorders; psoriasis colon cancer, leukemia, AIDS; thalamus disorders; metabolic disorders including diabetes and obesity; lung diseases such as asthma, emphysema, cystic fibrosis, and cancer; pancreatic disorders including pancreatic insufficiency and cancer; and prostate disorders including prostate cancer, as well as other diseases, disorders and conditions.

Costimulatory interactions between the B7 family ligands and their receptors play critical roles in the growth, differentiation, and death of T cells. Engagement of the T-cell costimulator CD28 by either specific antibodies or its natural ligands B7-1 and B7-2 increases antigen-specific proliferation of CD4⁺ T cells, enhances production of cytokines, induces maturation of CD8⁺ effector T cells and promotes T-cell survival.

NOV4 is homologous to the B7-H1 like proteins. Thus, NOV4 nucleic acids, polypeptides, antibodies and related compounds according to the invention will be useful in therapeutic and diagnostic applications implicated in, for example, the treatment of patients suffering from brain disorders including epilepsy, eating disorders, schizophrenia, ADD, and cancer; heart disease; inflammation and autoimmune disorders including Crohn's disease, IBD, allergies, rheumatoid and osteoarthritis, inflammatory skin disorders, blood disorders; psoriasis colon cancer, leukemia, AIDS; thalamus disorders; metabolic disorders including diabetes and obesity; lung diseases such as asthma, emphysema, cystic fibrosis and cancer; pancreatic disorders including pancreatic insufficiency and cancer; and prostate disorders including prostate cancer and other diseases, disorders and conditions of the like.

Several recent studies demonstrate the importance of the co-stimulatory interaction of B7 family members like B7RP-1 (B7 Related Protein-1), B7-1, and B7-2, with antigen receptors such as CD28, CTLA-4 (Cytotoxic T Lymphocyte-associated Antigen 4) and ICOS (Inducible Co-Stimulatory molecule). These protein interactions have been shown to be critical for normal T-cell activation and proliferation, B-cell stimulation and antibody production, immunoglobulin class switching, interleukin production, and germinal center formation.

NOV5 is homologous to the Prostasin protein family. Thus NOV5 nucleic acids, polypeptides, antibodies and related compounds according to the invention will be useful in therapeutic and diagnostic applications implicated in, for example, the treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation and other diseases, disorders and conditions of the like.

Human seminal fluid contains a variety of proteolytic enzymes, including prostate-specific antigen (OMIM #176820) and acrosin (OMIM #102480). These enzymes are involved in the postejaculatory hydrolysis of proteins and in semen coagulation and liquefaction (Yu et al. (1995)) obtained partial amino acid sequence of a 40-kD protein isolated from seminal fluid originally by Yu et al. (1994). The protein, designated serine protease-8 (gene symbol=PRSS8), was called prostasin by the authors. The precursor, proprostasin, is cleaved between residues 12 and 13 to produce a 12-amino acid light chain and a 299-amino acid heavy chain which are associated through a disulfide bond. The predicted amino acid sequence is between 34 and 42% identical to human acrosin, plasma kallikrein (OMIM #229000), and hepsin (OMIM #142440).

NOV6 is homologous to the Lysosomal acid lipase family of proteins. Thus NOV6 nucleic acids, polypeptides, antibodies and related compounds according to the invention will be useful in therapeutic and diagnostic applications implicated in, for example, Wolman disease and cholesteryl ester storage disease.

Lysosomal acid lipase-A (LIPA), the enzyme deficient in the presumably allelic Wolman disease and cholesterol ester storage disease (OMIM #278000), is located on chromosome 10. The distinct kinetic and physical properties of lipases A and B were defined by Warner et al. (1980). They stated that the natural substrate for LIPB is not known, and that it is not clear that LIPB is a lysosomal hydrolase. LIPA may serve an important role in cellular metabolism by releasing cholesterol. The liberated cholesterol suppresses further cholesterol synthesis and stimulates esterification of cholesterol within the cell.

NOV7 is homologous to Tryptase 4. Thus, the NOV7 nucleic acids, polypeptides, antibodies and related compounds according to the invention will be useful in therapeutic and diagnostic applications implicated in, for example, the treatment of patients suffering from: diabetes, Von Hippel-Lindau (VHL) syndrome, pancreatitis, obesity, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, transplantation, fertility, endometriosis, Hirschsprung's disease , Crohn's Disease, appendicitis and other diseases, disorders and conditions of the like.

Human tryptase is a structurally unique and mast cell specific trypsin-like serine protease. Recent biological and immunological investigations have implicated tryptase as a mediator in the pathology of numerous allergic and inflammatory conditions including rhinitis, conjunctivitis, and most notably asthma. A growing body of data further implicates tryptase in certain gastrointestinal, dermatological, and cardiovascular disorders as well. The recent availability of potent, and selective tryptase inhibitors, though, has facilitated the validation of this protease as an important therapeutic target as well.

NOV8 is homologous to the P450 family of proteins. Since the NOV8 protein of the invention is ubiquitously expressed in many tissues, the NOV8 nucleic acids and polypeptides, antibodies and related compounds according to the invention will be useful in therapeutic and diagnostic applications implicated in the treatment certain pathologies and disorders.

The P450 gene superfamily is a biologically diverse class of oxidase enzymes; members of the class are found in all organisms. P450 proteins are clinically and toxicologically important in humans; they are the principal enzymes in the metabolism of drugs and xenobiotic compounds, as well as in the synthesis of cholesterol, steroids and other lipids. Induction of some P450 genes can also be a risk factor for several types of cancer. This diversity of function is mirrored in the diversity of nucleotide and protein sequences; there are currently over 100 human P450 forms described. Allelic forms of many cytochrome P450 genes have been identified as causing quantitatively different rates of drug metabolism, and hence are important to consider in the development of safe and effective human pharmaceutical therapies. See, e.g., review in E. Tanaka, J Clinical Pharmacy & Therapeutics 24:323-329, 1999.

NOV9 is homologous to the Mitsugumin 29 protein. Thus, the NOV9 nucleic acids, polypeptides, antibodies and related compounds according to the invention will be useful in therapeutic and diagnostic applications implicated in, for example, the treatment of patients suffering from: Wiskott-Aldrich syndrome, Aldrich syndrome, eczema-thrombocytopenia-immunodeficiency syndrome, thrombocytopenia, night blindness, amyotrophic lateral sclerosis, Batten disease, ceroid lipofuscinosis, Rett syndrome, Pick disease (lobar atrophy), cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection and other diseases, disorders and conditions of the like.

Mitsugumin29 is involved in the formation of specialized endoplasmic reticulum systems in skeletal muscle and renal tubule cells. The subcellular distribution and protein structure suggest that mitsugumin29 is involved in communication between the T-tubular and junctional SR membranes.

NOV10 is homologous to members of Micromolar calcium-activated neutral protease 1 family of proteins. Thus, the NOV10 nucleic acids, polypeptides, antibodies and related compounds according to the invention will be useful in therapeutic and diagnostic applications implicated in, for example, the treatment of patients suffering from: diabetes, Von Hippel-Lindau (VHL) syndrome, pancreatitis, obesity, hypercalceimia, ulcers, endometriosis, fertility, hemophilia, hypercoagulation, idiopathic thrombocytopenic purpura, autoimmume disease, allergies, immunodeficiencies, transplantation, graft versus host disease, psoriasis, actinic keratosis, tuberous sclerosis, acne, hair growth/loss, allopecia, pigmentation disorders, endocrine disorders, hemophilia, lymphaedema, and other diseases, disorders and conditions of the like.

The predicted sequence described here belongs to the calpain protease family. The calpains, or calcium-activated neutral proteases, are nonlysosomal intracellular cysteine proteases (Richard, et al.). Calpain is an intracellular protease involved in many important cellular functions that are regulated by calcium.

NOV11 is homologous to the P2X2C-like proteins. Thus, the NOV11 nucleic acids, polypeptides, antibodies and related compounds according to the invention will be useful in therapeutic and diagnostic applications implicated in, for example, the treatment of patients suffering from pain, since P2X receptor activation of sensory neurones has been demonstrated in in vivo pain models, including the rat hindpaw and knee-joint preparations, as well as in inflammatory models. P2X4 and/or P2X6 receptors in the CNS also seem to be involved in pain pathways. Non-nociceptive P2 receptors on sensory nerves are present in muscle and on sensory endings in the heart and lung that initiate reflex activity involving vagal afferent and efferent nerve fibres (Br J Anaesth 2000 Apr;84(4):476-88). The compositions of the present invention may also have efficacy for treatment of patients suffering from diabetes, obesity, syndrome X, and other diseases, disorders and conditions of the like.

NOV12 is related to the DIABLO-like proteins. Thus, the NOV12 nucleic acids, polypeptides, antibodies and related compounds according to the invention will be useful in therapeutic and diagnostic applications implicated in, for example, the treatment of patients suffering from: cancer, trauma, bacterial and viral infections, regeneration (in vitro and in vivo), fertility, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, Von Hippel-Lindau (VHL) syndrome, tuberous sclerosis, endocrine disorders, Alzheimer's disease, stroke, hypercalceimia, Parkinson's disease, Huntington's disease, cerebral palsy, epilepsy, multiple sclerosis, ataxia-telangiectasia, leukodystrophies, behavioral disorders, addiction, anxiety, pain, neuroprotection and other diseases, disorders and conditions of the like.

The DIABLO protein (direct IAP binding protein with low pI) performs a critical function in apoptosis by eliminating the inhibitory effect of IAPs (inhibitor of apoptosis proteins) on caspases (1). This protein is also known as Smac for second mitochondria-derived activator of caspase. DIABLO/Smac is normally a mitochondrial protein but is released into the cytosol when cells undergo apoptosis. Mitochondrial import and cleavage of its signal peptide are required for DIABLO/Smac to gain its apoptotic activity. In addition, overexpression of DIABLO/Smac has been shown to increase cellular sensitivity to apoptotic stimuli (2).

NOV13 is homologous to the HRPET-1 related protein. Thus, the NOV13 nucleic acids, polypeptides, antibodies and related compounds according to the invention will be useful in therapeutic and diagnostic applications implicated in, for example, the treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, diabetes, Von Hippel-Lindau (VHL) syndrome, pancreatitis, fertility, endometriosis, xerostomia, cirrhosis, hemophilia, hypercoagulation, idiopathic thrombocytopenic purpura, autoimmume disease, allergies, immunodeficiencies, graft versus host disease, lymphedema, hemophilia, hypercoagulation, Alzheimer's disease, stroke, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, systemic lupus erythematosus, asthma, emphysema, scleroderma, ARDS, psoriasis, actinic keratosis, acne, hair growth/loss, allopecia, pigmentation disorders, endocrine disorders, renal artery stenosis, interstitial nephritis, glomerulonephritis, polycystic kidney disease, systemic lupus erythematosus, renal tubular acidosis, IgA nephropathy, Lesch-Nyhan syndrome and other diseases, disorders and conditions of the like.

The HRPET-1 related protein is highly conserved across species, among C. elegans, Drosophila, mouse and human. It is predicted to be membrane associated. The high conservation in primary sequences indicates that it has important biological functions, although currently unknown. The HRPET-1 related protein also shows homology with plant adhesion molecules, suggesting that the HRPET-1 related protein is likely a cell adhesion molecule involved in cell interaction and migration.

NOV14 is homologous to B7-H2B protein. Thus, the NOV14 nucleic acids, polypeptides, antibodies and related compounds according to the invention will be useful in therapeutic and diagnostic applications implicated in, for example, the treatment of patients suffering from: brain disorders including epilepsy, eating disorders, schizophrenia, ADD, and cancer; heart disease; inflammation and autoimmune disorders including Crohn's disease, IBD, allergies, rheumatoid and osteoarthritis, inflammatory skin disorders, allergies, blood disorders; psoriasis colon cancer, leukemia AIDS; thalamus disorders; metabolic disorders including diabetes and obesity; lung diseases such as asthma, emphysema, cystic fibrosis, and cancer; pancreatic disorders including pancreatic insufficiency and cancer; and prostate disorders including prostate cancer and other diseases, disorders and conditions of the like.

Costimulatory interactions between the B7 family ligands and their receptors play critical roles in the growth, differentiation, and death of T cells. Engagement of the T-cell costimulator CD28 by either specific antibodies or its natural ligands B7-1 and B7-2 increases antigen-specific proliferation of CD4⁺ T cells, enhances production of cytokines, induces maturation of CD8⁺ effector T cells and promotes T-cell survival. Signaling through homologous CTLA-4 receptor of B7-1 and B7-2 on activated T cells, however, is thought to deliver a negative signal that inhibits T-cell proliferation, interleukin (IL)-2 production, and cell cycle progression.

NOV15 is homologous to galactosyltransferase-like proteins. Thus, the NOV15 nucleic acids, polypeptides, antibodies and related compounds according to the invention will be useful in therapeutic and diagnostic applications implicated in, for example, the treatment of patients suffering from: proteodermatan sulfate, defective biosynthesis of PDS, defective biosynthesis of dermatan sulfate proteoglycan xylosylprotein 4-beta-galactosyltransferase deficiency xgpt deficiency galactosyltransferase I deficiency, Ehlers-Danlos syndrome, cardiomyopathy, Atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, endometriosis, fertility, Von Hippel-Lindau (VHL) syndrome, Alzheimer's disease, Stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection and other diseases, disorders and conditions of the like.

The enzyme galactosyltransferase (EC 2.4.1.38) catalyzes the reaction involving UDP-galactose and N-acetylglucosamine for the production of galactose beta-1,4-N-acetylglucosamine. The galactosyltransferase enzyme can also form a heterodimer with the regulatory protein alpha-lactalbumin to form lactose synthetase (EC 2.4.1.22). In addition to a biosynthetic role, galactosyltransferases may be components of plasma membranes where they may function in intercellular recognition and/or adhesion.

NOV16 is homologous to Lymphocyte antigen precursor-like proteins. Thus, the NOV16 nucleic acids, polypeptides, antibodies and related compounds according to the invention will be useful in therapeutic and diagnostic applications implicated in, for example, the treatment of patients suffering from cancer,trauma, regeneration, viral/bacterial/parasitic infections.

NOV17 is homologous to Pepsinogen C-like proteins. Thus, the NOV17 nucleic acids, polypeptides, antibodies and related compounds according to the invention will be useful in therapeutic and diagnostic applications implicated in, for example, the treatment of patients suffering from: ulcer, hypertension (Scand J Clin Lab Invest Suppl 1992;210:111-9), gastric mucosal inflammation and atrophy, and other diseases, disorders and conditions of the like. PGC gene polymorphism has been associated with gastric ulcer and can be a subclinical marker of the genetic predisposition to gastric ulcer (Nippon Rinsho 1996 Apr;54(4): 1149-54). The serum determination of pepsinogen A (PGA) and pepsinogen C (PGC) might indicate gastric mucosal inflammation and atrophy. Body gastric mucosa produces both PGA and PGC, while antral mucosa produces only PGC. Therefore, diseases involving mainly the antrum, such as H. pylori infection, are mainly indicated by the variations in serum PGC than in serum PGA. In agreement, when the antral mucosa is infected by the more virulent cagA positive H. pylori strains, which cause severe inflammation, serum PGC significantly increases (Recenti Prog Med 1999 Jun;90(6):342-6).

The gastric aspartic proteinases (pepsin A, pepsin B, gastricsin/pepsinogen C and chymosin) are synthesized in the gastric mucosa as inactive precursors, known as zyrnogens. The gastric zymogens each contain a prosegment (i.e. additional residues at the N-terminus of the active enzyme) that serves to stabilize the inactive form and prevent entry of the substrate to the active site. Upon ingestion of food, each of the zymogens is released into the gastric lumen and undergoes conversion into active enzyme in the acidic gastric juice.

NOV18 is homologous to ALR-like proteins. Thus, the NOV18 nucleic acids, polypeptides, antibodies and related compounds according to the invention will be useful in therapeutic and diagnostic applications implicated in, for example, the treatment of patients suffering from: cancers such as acute lymphoid leukemia, acute myeloid leukemia, translocation-associated leukemias, and other diseases, disorders and conditions of the like.

The ALL-1 gene is involved in human acute leukemia through chromosome translocations or internal rearrangements. ALL-1 is the human homologue of Drosophila trithorax. The latter is a member of the trithorax group (trx-G) genes which together with the Polycomb group (Pc-G) genes act as positive and negative regulators, respectively, to determine the body structure of Drosophila. ALR, a ALL-1 related protein, which encodes a gigantic 5262 amino acid long protein containing a SET domain, five PHD fingers, potential zinc fingers, and a very long run of glutamines interrupted by hydrophobic residues, mostly leucine.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopolesis, wound healing and angiogenesis.

Additional utilities for the NOVX nucleic acids and polypeptides according to the invention are disclosed herein.

NOV1

One NOVX protein of the invention, referred to herein as NOV1, includes four fibromodulin-like proteins. The disclosed proteins have been named NOV1a, NOV1b, NOV1c and NOV1d.

NOV1a

A disclosed NOV1a (designated CuraGen Acc. No. CG-56201-01), which encodes a novel fibromodulin-like protein and includes the 1455 nucleotide sequence (SEQ ID NO: 1) is shown in Table 1A. An open reading frame for the mature protein was identified beginning with an ATG initiation codon at nucleotides 197-199 and ending with a TGA stop codon at nucleotides 1445-1447. Putative untranslated regions are underlined in Table 1A, and the start and stop codons are in bold letters.

TABLE 1A
NOV1a Nucleotide Sequence

(SEQ ID NO:1)

CCTGCAGGTTCTCTCAGCCCCTTTTCACAATATTTGATTAGGAATTTGGGGCGGGACCCTGGTCTGGCACAGGCACGCACACTC
TCAGTAGACTCTTTCACTCCTCTCTCTCTTCCTCTCTCACACGTTCTCCAACCCAAGGAGGCCAGACAGAGGGACGTGGTCACT
CTCTGAAAAGTTCAACTTGAGAGACAAAATGCAGTGGACCTCCCTCCTGCTGCTGGCAGGGCTCTTCTCCCTCTCCCAGGCCCA
GTATGAAGATGACCCTCATTGGTGGTTCCACTACCTCCGCAGCCAGCAGTCCACCTACTACGATCCCTATGACCCTTACCCGTA
TGAGACCTACGAGCCTTACCCCTATGGGGTGGATGAAGGGCCAGCCTACACCTACGGCTCTCCATCCCCTCCAGATCCCCGCGA
CTGCCCCCAGGAATGCGACTGCCCACCCAACTTCCCCACGGCCATGTACTGTGACAATCGCAACCTCAACTACCTGCCCTTCGT
TCCCTCCCGCATGAAGTATGTGTACTTCCAGAACAACCAGATCACCTCCATCCAGGAAGGCGTCTTTGACAATGCCACAGGGCT
GCTCTGGATTGCTCTCCACGGCAACCAGATCACCAGTGATAAGGTGGGCAGGAAGGTCTTCTCCAAGCTGAGGCACCTGGAGAG
GCTGTACCTGGACCACAACAACCTGACCCGGATGCCCGGTCCCCTGCCTCGATCCCTGAGAGAGCTCCATCTCGACCACAACCA
GATCTCACGGGTCCCCAACAATGCTCTGGAGGGGCTGGAGAACCTCACGGCCTTGTACCTCCAACACAATGAGATCCAGGAAGT
GGGCAGTTCCATGAGGGGCCTCCGGTCACTGATCTTGCTGGACCTGAGTTATAACCACCTTCGGAAGGTGCCTGATGGGCTGCC
CTCAGCTCTTGAGCAGCTGTACATGGAGCACAACAATGTCTACACCGTCCCCGATAGCTACTTCCGGGGGGCGCCCAAGCTGCT
GTATGTGCGGCTGTCCCACAACAGTCTAACCAACAATGGCCTGGCCTCCAACACCTTCAATTCCAGCAGCCTCCTTGAGCTAGA
CCTCTCCTACAACCAGCTGCAGAAGATCCCCCCAGTCAACACCAACCTGGAGAACCTCTACCTCCAAGGCAATAGGATCAATGA
GTTCTCCATCCAGGAAGGCGTCTTTGACAATGCCACAGGGCTGCTCTGGATTGCTCTCCACGGCAACTTCTCCACGGCCATGTA
CTGTGACAATCGCAACCTCAAGTACCTGCCCTTCGTTCCCTCCCGCATGAAGTATGTGTACTTCCAGAACAACCAGATCACCTC
CAAGCTGCAGGTGCTGCGCCTGGACGGGAACGAGATCAAGCGCAGCGCCATGCCTGCCGACGCGCCCCTCTGCCTGCGCCTTGC
CAGCCTCATCGAGATCTGAGCAGCCCT

The disclosed NOV1a nucleic acid sequence maps to chromosome 1 and has 1050 of 1061 bases (98%) identical to a gb:GENBANK-ID:HSFIBR|acc:X72913.1 mRNA from Homo sapiens (H.sapiens gene for fibromodulin) (E=2.0e⁻²⁴⁶).

The NOV1a polypeptide (SEQ ID NO: 2) is 416 amino acid residues in length and is presented using the one-letter amino acid code in Table 1B. The SignalP, Psort and/or Hydropathy results predict that NOV1a has a signal peptide and is likely to be localized to the lysosome (lumen) with a certainty of 0.5500. In alternative embodiments, a NOV1a polypeptide is located outside the cell with a certainty of 0.3700, the endoplasmic reticulum (membrane) with a certainty of 0.1000, or in the endoplasmic reticulum (lumen) with a certainty of 0.1000. The SignaIP predicts a likely cleavage site for a NOV1a peptide between amino acid positions 18 and 19, i.e. at the dash in the sequence SQA-QY.

TABLE 1B
Encoded NOV1a Protein Sequence

(SEQ ID NO:2)

MQWTSLLLLAGLFSLSQAQYEDDPHWWFHYLRSQQSTYYDPYDPYPYETYEPYPYGVDEGPAYTYGSPSPPDPRD
CPQECDCPPNFPTAMYCDNRNLKYLPFVPSRMKYVYFQNNQITSIQEGVFDNATGLLWIALHGNQITSDKVGRKV
FSKLRHLERLYLDHNNLTRMPGPLPRSLRELHLDHNQISRVPNNALEGLENLTALYLQHNEIQEVGSSMRGLRSL
ILLDLSYNHLRKVPDGLPSALEQLYMEHNNVYTVPDSYFRGAPKLLYVRLSHNSLTNNGLASNTFNSSSLLELDL
SYNQLQKIPPVNTNLENLYLQGNRINEFSIQEGVFDNATGLLWIALHGNFSTAMYCDNRNLKYLPFVPSRMKYVY
FQNNQITSKLQVLRLDGNEIKRSAMPADAPLCLRLASLIEI

the NOV1a amino acid sequence have 336 of 353 amino acid residues (95%) identical to, and 338 of 353 amino acid residues (95%) similar to, the 376 amino acid residue ptnr:SWISSNEW-ACC:Q06828 protein from Homo sapiens (Human) (FIBROMODULIN PRECURSOR (FM) (COLLAGEN-BINDING 59 KDA PROTEIN)) (E=3.9e-¹⁸⁴).

Possible small nucleotide polymorphisms (SNPs) found for NOV1a are listed in Tables 1C.

TABLE 1C
SNPs

| | Nucleotide | | Amino Acid | |
|---|---|---|---|---|
| Variant | Position | Base Change | Position | Base Change |
| c110.2794 | 190 | Insertion of A | NA | NA |
| c110.2793 | 192 | Insertion of A | NA | NA |
| 13374277 | 433 | A > G | NA | NA |
| 13374280 | 447 | C > T | 84 | Pro > Leu |
| 13374278 | 589 | G > C | NA | NA |
| 13374281 | 785 | G > A | 197 | Glu > Lys |
| 13374279 | 1002 | A > G | 269 | Lys > Arg |

NOV1b

A disclosed NOV1b (designated CuraGen Acc. No. CG56201-02), which includes the 965 nucleotide sequence (SEQ ID NO: 3) shown in Table 1D. An open reading frame for the mature protein was identified beginning with an ATG codon at nucleotides 57-59 and ending with a TGA codon at nucleotides 963-965. The start and stop codons of the open reading frame are highlighted in bold type. Putative untranslated regions are underlined.

TABLE 1D
NOV1b Nucleotide Sequence

(SEQ ID NO:3)

AAGGAGGCCAGACAGAGGGACGTGGTCACTTCTCTGAAAAGTTCAACTTGAGCAAAATGCAGTGGACCTCCCTCCTGCTGC
TGGCAGGGCTCTTCTCCCTCTCCCAGGCCCAGTATGAAGATGACCCTCATTGGTGGTTCCACTACCTCCGCAGCCAGCAGT
CCACCTACTACGATCCCTATGACCCTTACCCGTATGAGACCTACGAGCCTTACCCCTATGGGGTGGATGAAGGGCCAGCCT
ACACCTACGGCTCTCCATCCCCTCCAGATCCCCGCGACTGCCCCCAGGAATGCGACTGCCCACCCAACTTCCCCACGGCCA
TGTACTGTGACAATCGCAACCTCAAGTACCTGCCTCGATCCCTGAGAGAGCTCCATCTCGACCACAACCAGATCTCACGGG
TCCCCAACAATGCTCTGGAGGGGCTGGAGAACCTCACGGCCTTGTACCTCCAACACAATGAGATCCAGGAAGTGGGCAGTT
CCATGAGGGGCCTCCGGTCACTGTACTTGCTGGACCTGAGTTATAACCACCTTCGGAAGGTGCCTGATGGGCTGCCCTCAG
CTCTTGAGCAGCTGTACATGGAGCACAACAATGTCTACACCGTCCCCGATAGCTACTTCCGGGGGGCGCCCAAGCTGCTGT
ATGTGCGGCTGTCCCACAACAGTCTAACCAACAATGGCCTGGCCTCCAACACCTTCAATTCCAGCAGCCTCCTTGAGCTAG
ACCTCTCCTACAACCAGCTGCAGAAGATCCCCCCAGTCAACACCAACCTGGAGAACCTCTACCTCCAAGGCAATAGGATCA
ATGAGTTCTCCATCAGCAGCTTCTGCACCGTGGTGGACGTCGTGAACTTCTCCCAGCTGCAGGTCGTGCGGCTGGACGGGA
ACGAGATGAAGCGGAGCGCCATGCCTGCCGAGGCGCCCCTCTGCCTGCGCCTTGCCAGCCTCATCGAGATCTGA

The disclosed NOV1b nucleic acid sequence maps to chromosome 1 and has 613 of 613 bases (100%) identical to a gb:GENBANK-ID:HSFIBR|acc:X72913.1 mRNA from Homo sapiens (H.sapiens gene for fibromodulin) (E=2.7e⁻²¹¹).

The NOV1b polypeptide (SEQ ID NO: 4) is 302 amino acid residues in length and is presented using the one-letter amino acid code in Table 1E. The SignalP, Psort and/or Hydropathy results predict that NOV1b has a signal peptide and is likely to be localized to the lysosome (lumen) with a certainty of 0.4595. In alternative embodiments, a NOV1b polypeptide is located to the outside of the cell with a certainty of 0.3700, the endoplasmic reticulum (membrane) with a certainty of 0.1000, or the endoplasmic reticulum (lumen) with a certainty of 0.1000. The SignalP predicts a likely cleavage site for a NOV1a peptide between amino acid positions 18 and 19, i.e. at the dash in the sequence SQA-QY.

TABLE 1E
Encoded NOV1b Protein Sequence

(SEQ ID NO:4)

MQWTSLLLLAGLFSLSQAQYEDDPHWWFHYLRSQQSTYYDPYDPYPYETYEPYPYGVDEGPAYTYGSPSPPDPRDCPQECDC
PPNFPTAMYCDNRNLKYLPRSLRELHLDHNQISRVPNNALEGLENLTALYLQHNEIQEVGSSMRGLRSLYLLDLSYNHLRKV
PDGLPSALEQLYMEHNNVYTVPDSYFRGAPKLLYVRLSHNSLTNNGLASNTFNSSSLLELDLSYNQLQKIPPVNTNLENLYL
QGNRINEFSISSFCTVVDVVNFSQLQVVRLDGNEMKRSAMPAEAPLCLRLASLIEI

The NOV1b amino acid sequence has 207 of 217 amino acid residues (95%) identical to, and 209 of 217 amino acid residues (96%) similar to, the 376 amino acid residue ptnr:SWISSNEW-ACC:Q06828 protein from Homo sapiens (Human) (FIBROMODULIN PRECURSOR (FM) (COLLAGEN-BINDING 59 KDA PROTEIN)) (E=7.2e⁻¹⁰⁵).

NOV1c

A disclosed NOV1c (designated CuraGen Acc. No. CG56201-04), which includes the 1139 nucleotide sequence (SEQ ID NO: 5) shown in Table 1F. An open reading frame for the mature protein was identified beginning with an ATG codon at nucleotides 57-59 and ending with a TGA codon at nucleotides 1137-1139. The start and stop codons of the open reading frame are highlighted in bold type. Putative untranslated regions are underlined.

TABLE 1F
NOV1c Nucleotide Sequence

(SEQ ID NO:5)

AAGGAGGCCAGACAGAGGGACGTGGTCACTTCTCTGAAAAGTTCAACTTGAGCAAAATGCAGTGGACCTCCCTCCTGCTGC
TGGCAGGGCTCTTCTCCCTCTCCCAGGCCCAGTATGAAGATGACCCTCATTGGTGGTTCCACTACCTCCGCAGCCAGCAGT
CCACCTACTACGATCCCTATGACCCTTACCCGTATGAGACCTACGAGCCTTACCCCTATGGGGTGGATGAAGGGCCAGCCT
ACACCTACGGCTCTCCATCCCCTCCAGATCCCCGCGACTGCCCCCAGGAGTGCGACTGCCCACCCAACTTCCCCACGGCCA
TGTACTGTGACAATCGCAACCTCAAGTACCTGCCCTTCGTTCCCTCCCGCATGAAGTATGTGTACTTCCAGAACAACCAGA
TCACCTCCATCCAGGAAGGCGTCTTTGACAATGCCACAGGGCTGCTCTGGATTGCTCTCCACGGCAACCAGATCACCAGTC
ATAAGGTGGGCAGGAAGGTCTTCTCCAAGCTGAGGCACCTGGAGAGGCTGTACCTGGACCACAACAACCTGACCCGGATGC
CCGGTCCCCTGCCTCGATCCCTGAGAGAGCTCCATCTCGACCACAACCAGATCTCACGGGTCCCCAACAATGCTCTGGAGG
GGCTGGAGAACCTCACGGCCTTGTACCTCCAACACAATGAGATCCAGGAAGTGGGCAGTTCCATGAGGGGCCTCCGGTCAC
TGTACTTGCTGGACCTGAGTTATAACCACCTTCGGAAGGTGCCTGATGGGCTGCCCTCAGCTCTTGAGCAGCTGTACATGC
AGCACAACAATGTCTACACCGTCCCCGATAGCTACTTCCGGGGGGCGCCCAAGCTGCTGTATGTGCGGCTGTCCCACAACA
GTCTAACCAACAATGGCCTGGCCTCCAACACCTTCAATTCCAGCAGCCTCCTTGAGCTAGACCTCTCCTACAACCAGCTGC
AGAAGATCCCCCCAGTCAACACCATCAGCAGCTTCTGCACCGTGGTGGACGTCGTGAACTTCTCCCAGCTGCAGGTCGTGC
GGCTGGACGGGAACGAGATGAAGCGGAGCGCCATGCCTGCCGAGGCGCCCCTCTGCCTGCGCCTTGCCAGCCTCATCGAGA
TCTGA

The nucleic acid sequence of NOV1c maps to chromosome 1 and has 1036 of 1065 bases (97%) identical to a gb:GENBANK-ID:HSFIBR|acc:X72913.1 mRNA from Homo sapiens (H.sapiens gene for fibromodulin) (E=9.4e⁻²²⁰).

The NOV1c polypeptide (SEQ ID NO: 6) is 360 amino acid residues in length and is presented using the one-letter amino acid code in Table 1G. The SignalP, Psort and/or Hydropathy results predict that NOV1c has a signal peptide and is likely to be localized to the lysosome (lumen) with a certainty of 0.5305. In alternative embodiments, a NOV1c polypeptide is located to the outside of the cell with a certainty of 0.3700, the endoplasmic reticulum (membrane) with a certainty of 0.1000, or the endoplasmic reticulum (lumen) with a certainty of 0.1000. The Signal predicts a likely cleavage site for a NOV1c peptide between amino acid positions 18 and 19, i.e. at the dash in the sequence SQA-QY.

TABLE 1G
Encoded NOV1c Protein Sequence

(SEQ ID NO:6)

MQWTSLLLLAGLFSLSQAQYEDDPHWWFHYLRSQQSTYYDPYDPYPYETYEPYPYGVDEGPAYTYGSPSPPDPRDCPQECDC
PPNFPTAMYCDNRNLKYLPFVPSRMKYVYFQNNQITSIQEGVFDNATGLLWIALHGNQITSDKVGRKVFSKLRHLERLYLDH
NNLTRMPGPLPRSLRELHLDHNQISRVPNNALEGLENLTALYLQHNEIQEVGSSMRGLRSLYLLDLSYNHLRKVPDGLPSAL
EQLYMEHNNVYTVPDSYFRGAPKLLYVRLSHNSLTNNGLASNTFNSSSLLELDLSYNQLQKIPPVNTISSFCTVVDVVNFSQ
LQVVRLDGNEMKRSAMPAEAPLCLRLASLIEI

The NOV1c amino acid sequence has 360 of 376 amino acid residues (95%) identical to, and 360 of 376 amino acid residues (95%) similar to, the 376 amino acid residue ptnr:SWISSNEW-ACC:Q06828 protein from Homo sapiens (Human) (FIBROMODULIN PRECURSOR (FM) (COLLAGEN-BINDING 59 KDA PROTEIN)) (E=1.4e-195).

NOV1c is expressed in at least the following tissues: aorta, bone marrow, brain, cartilage, cochlea, colon, heart, kidney, liver, lung, lymph node, lymphoid tissue, mammary gland/breast, muscle, ovary, pancreas, parathyroid gland, parotid salivary glands, placenta, prostate, retina, salivary glands, skin, spinal chord, stomach, testis, thyroid, uterus, whole organism.

NOV1d

A disclosed NOV1d (designated CuraGen Acc. No. CG56201-01; Assembly 224700033), which includes the 1053 nucleotide sequence (SEQ ID NO: 7) shown in Table 1H. An open reading frame for the mature protein was identified beginning with an GGA codon at nucleotides 1-3 and ending with a GAG codon at nucleotides 1051-1053. The start and stop codons of the open reading frame are highlighted in bold type.

TABLE 1H
NOV1d Nucleotide Sequence

(SEQ ID NO:7)

GGATCCCAGTATGAAGATGACCCTCATTGGTGGTTCCACTACCTCCGCAGCCAGCAGTCCACCTACTACGATCCCTATGAC
CCTTACCCGTATGAGACCTACGAGCCTTACCCCTATGGGGTGGATGAAGGGCCAGCCTACACCTACGGCTCTCCATCCCCT
CCAGATCCCCGCGACTGCCCCCAGGAATGCGACTGCCCACCCAACTTCCCCACGGCCATGTACTGTGACAATCGCAACCTC
AAGTACCTGCCCTTCGTTCCCTCCCGCATGAAGTATGTGTACTTCCAGAACAACCAGATCACCTCCATCCAGGAAGGCGTC
TTTGACAATGCCACAGGGCTGCTCTGGATTGCTCTCCACGGCAACCAGATCACCAGTGATAAGGTGGGCAGGAAGGTCTTC
TCCAAGCTGAGGCACCTGGAGAGGCTGTACCTGGACCACAACAACCTGACCCGGATGCCCGGTCCCCTGCCTCGATCCCTG
AGAGAGCTCCATCTCGACCACAACCAGATCTCACGGGTCCCCAACAATGCTCTGGAGGGGCTGGAGAACCGCACGGCCTTG
TACCTCCAACACAATGAGATCCAGGAAGTGGGCAGTTCCATGAGGGGCCTCCGGTCACTGATCTTGCTGGACCTGAGTTAT
AACCACCTTCGGAAGGTGCCTGATGGGCTGCCCTCAGCTCTTGAGCAGCTGTACATGGAGCACAACAATGTCTACACCGTC
CCCGATAGCTACTTCCGGGGGGCGCCCAAGCTGCTGTATGTGCGGCTGTCCCACAACAGTCTAACCAACAATGGCCTGGCC
TCCAACACCTTCAATTCCAGCAGCCTCCTTGAGCTAGACCTCTCCTACAACCTGGAGAACCTCTACCTCCAAGGCAATAGG
ATCAATGAGTTCTCCATCAGCAGCTTCTGCACCGTGGTGGACGTCGTGAACTTCTCCAAGCTGCAGGTGCTGCGCCTGGAC
GGGAACGAGATCAAGCGCAGCGCCATGCCTGCCGACGCGCCCCTCTGCCGGCGCCTTGCCAGCCTCATCGAGATCCTCGAG

The NOV1d polypeptide (SEQ ID NO: 8) is 360 amino acid residues in length and is presented using the one-letter amino acid code in Table 1I. The SignalP, Psort and/or Hydropathy results predict that NOV1c has a signal peptide and is likely to be localized to the lysosome (lumen) with a certainty of 0.5305. In alternative embodiments, a NOV1c polypeptide is located to the outside of the cell with a certainty of 0.3700, the endoplasmic reticulum (membrane) with a certainty of 0.1000, or the endoplasmic reticulum (lumen) with a certainty of 0.1000. The SignaIP predicts a likely cleavage site for a NOV1c peptide between amino acid positions 18 and 19, i.e. at the dash in the sequence SQA-QY.

TABLE 1I
Encoded NOV1c Protein Sequence

(SEQ ID NO:8)

GSQYEDDPHWWFHYLRSQQSTYYDPYDPYPYETYEPYPYGVDEGPAYTYGSPSPPDPRDCPQECDCPPNFPTAMYCDNRNLK
YLPFVPSRMKYVYFQNNQITSIQEGVFDNATGLLWIALHGNQITSDKVGRKVFSKLRHLERLYLDHNNLTRMPGPLPRSLRE
LHLDHNQISRVPNNALEGLENRTALYLQHNEIQEVGSSMRGLRSLILLDLSYNHLRKVPDGLPSALEQLYMEHNNVYTVPDS
YFRGAPKLLYVRLSHNSLTNNGLASNTFNSSSLLELDLSYNLENLYLQGNRINEFSISSFCTVVDVVNFSKLQVLRLDGNEI
KRSAMPADAPLCRRLASLIEILE

NOV1c is expressed in at least the following tissues: aorta, bone marrow, brain, cartilage, cochlea, colon, heart, kidney, liver, lung, lymph node, lymphoid tissue, mammary gland/breast, muscle, ovary, pancreas, parathyroid gland, parotid salivary glands, placenta, prostate, retina, salivary glands, skin, spinal cord, stomach, testis, thyroid, uterus, whole oraganism. Expression information was derived from the tissue sources of the sequences that were included in the derivation of the sequence of NOV1d.

NOV1a, NOV1b, NOV1c and NOV1d are very closely homologous as is shown in the amino acid alignment in Table 1J.

[image]

[image]

Homologies to any of the above NOV1 proteins will be shared by the other NOV1 proteins insofar as they are homologous to each other as shown above. Any reference to NOV1 is assumed to refer to both of the NOV1 proteins in general, unless otherwise noted.

NOV1a also has homology to the amino acid sequences shown in the BLASTP data listed in Table 1K.

TABLE 1K
BLAST results for NOV1

| Gene Index/ | | Length | Identity | Positives | |
|---|---|---|---|---|---|
| Identifier | Protein/Organism | (aa) | (%) | (%) | Expect |
| gi\|11424102\|ref\| | similar to | 376 | 336/416 | 338/416 | e−174 |
| XP_001782.1\| | FIBROMODULIN | | (80%) | (80%) | |
| (XM_001782) | PRECURSOR (FM) | | | | |
| | (COLLAGEN-BINDING | | | | |
| | 59 KDA PROTEIN) | | | | |
| | [Homo sapiens] | | | | |
| gi\|4503763\|ref\|N | fibromodulin | 376 | 332/416 | 336/416 | e−172 |
| P_002014.1\| | precursor [Homo | | (79%) | (79%) | |
| (NM_002023) | sapiens] | | | | |
| gi\|544335\|sp\|Q06 | FIBROMODULIN | 376 | 331/416 | 337/416 | e−171 |
| 828\|FMOD_HUMAN | PRECURSOR (FM) | | (79%) | (80%) | |
| | (COLLAGEN-BINDING | | | | |
| | 59 KDA PROTEIN) | | | | |
| | [Homo sapiens] | | | | |
| gi\|1706877\|sp\|P5 | FIBROMODULIN | 376 | 314/416 | 327/416 | e−161 |
| 0609\|FMOD_RAT | PRECURSOR (FM) | | (75%) | (78%) | |
| | [Rattus norvegicus] | | | | |
| gi\|10946680\|ref\| | fibromodulin [Mus | 376 | 313/416 | 329/416 | e−161 |
| NP_067330.1\| | musculus] | | (75%) | (78%) | |
| (NM_021355) | | | | | |

The homology of these sequences is shown graphically in the ClustalW analysis shown in Table 1L.

[image]

[image]

Tables 1M and 1N list the domain description from DOMAIN analysis results against NOV1. This indicates that the NOV1 sequence has properties similar to those of other proteins known to contain these domains.

TABLE 1M
Domain Analysis of NOV1

gnl|Smart|smart00013, LRRNT, Leucine rich repeat
N-terminal domain (SEQ ID NO:62)
Length = 34 residues, 100% aligned
Score = 45.8 bits (107), Expect = 5e−06

| | | | |
|---|---|---|---|
| Query: | 75 | DCPQECDCPPNFPTAMYCDNRNLKYLPF-VPSRMKY | 109 |
| | | \|\| \|+\| \| \|\|+ \| \| \| +\| +\|+ | |
| Sbjct: | 1 | ACPAPCNCSP--GTAVDCSGRCLTEVPLDLPADTTL | 34 |

TABLE 1N
Domain Analysis of NOV1

gnl|Pfam|pfam01462, LRRNT, Leucine rich repeat
N-terminal domain (SEQ ID NO:63)
Length = 28 residues, 100% aligned
Score = 42.4 bits (107). Expect = 5e−05

| | | | |
|---|---|---|---|
| Query: | 75 | DCPQECDCPPNFPTAMYCDNRNLKYLPFVPS | 105 |
| | | \|\|+ \| \| \| + \| \| \| +\| | |
| Sbjct: | 1 | ACPRPCHCSG---TVVDCSGRGLTEVPRDLP | 28 |

Leucine rich repeat N-terminal domain (Accno. gnl|Pfam|pfam01462) is represented by the sequence domain LRRNT. Leucine Rich Repeats pfam00560 are short sequence motifs present in a number of proteins with diverse functions and cellular locations. Leucine Rich Repeats are often flanked by cysteine rich domains. This domain is often found at the N-terminus of tandem leucine rich repeats.

The fibromodulin precursor precursor (collagen-binding 59 kd protein) binds to type I and type II collagen and affects the rate of fibrils formation. It also binds keratan sulfate chains and belongs to the small interstitial proteoglycans family. This protein also contains 10 repeated leucine-rich (lrr) segments.

Fibromodulin is a member of a family of small interstitial proteoglycans that also includes decorin (DCN; OMIM 125255), biglycan (BGN; OMIM 301870), and lumican (LDC; OMIM 600616). The core proteins of these proteoglycans are structurally related, consisting of a central region composed of leucine-rich repeats flanked by disulfide-bonded terminal domains, with that for fibromodulin possessing up to 4 keratan sulfate chains within its leucine-rich domain. Fibromodulin exhibits a wide tissue distribution, with the highest abundance observed in articular cartilage, tendon, and ligament. It has been suggested that fibromodulin participates in the assembly of the extracellular matrix by virtue of its ability to interact with type I and type II collagen fibrils and to inhibit fibrillogenesis in vitro.

The protein similarity information, expression pattern, cellular localization, and map location for the NOV1 protein and nucleic acid disclosed herein suggest that it may have important structural and/or physiological functions characteristic of the Glycoprotein family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from: repair of damage to cartilage and ligaments; therapeutic applications to joint repair, as well as other diseases, disorders and conditions.

The novel nucleic acid encoding the fibromodulin-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV1 protein has multiple hydrophilic regions, each of which can be used as an immunogen. In one embodiment, a contemplated NOV1 epitope is from about amino acids 30 to 32. In another embodiment, a contemplated NOV1 epitope is from about amino acids 45 to 49. In other specific embodiments, contemplated NOV1 epitopes are from about amino acids 65 to 80, 105 to 120, 140 to 150, 155 to 180, 190 to 192, 198 to 200, 210 to 215, 220 to 225, 230 to 250 and 280 to 300.

NOV2

NOV2 includes three novel secretin receptor precursor-like proteins. The disclosed proteins have been named NOV2a, NOV2b, and NOV2c.

NOV2a

A disclosed NOV2a nucleic acid (designated as CuraGen Acc. No. CG56213-01), which encodes a novel secretin receptor precursor-like protein includes the 1280 nucleotide sequence (SEQ ID NO: 9) shown in Table 2A. An open reading frame for the mature protein was identified beginning with and ACT codon at nucleotides 1-3 and ending with a TGA codon at nucleotides 1264-1266. Putative untranslated regions are underlined in Table 2A, and the start and stop codons are in bold letters.

TABLE 2A
NOV2a Nucleotide Sequence

(SEQ ID NO:9)

ACTGGAGCTCTTCCCCGACTATGTGACGTGCTACAAGTGCTGTGGGAAGAGCAAGACCAGTGCCTGCAGGAACTCTCCAGAGAG
CAGACAGGAGACCTGGGCACGGAGCAGCCAGTGCCAGGTTGTGAGGGGATGTGGGACAACATAAGCTGCTGGCCCTCTTCTGTG
CCGGGCCCGATGGTGGAGGTGGAATGCCCGAGATTCCTCCGGATGCTCACCAGCAGAAATGGTTCCTTGTTCCGAAACTGCACA
CAGGATGGCTGGTCAGAAACCTTCCCCAGGCCTAATCTGGCCTGTGGCGTTAATGTGAACGACTCTTCCAACGAGAAGCGGCAC
TCCTACCTGCTGAAGCTCAAAGTCATGTACACCGTGGGCTACAGCTCCTCCCTGGTCATGCTCCTGGTCGCCCTTGGCATCCTC
TGTGCTTTCCGGAGGCTCCACTGCACTCGCAACTACATCCACATGCACCTGTTCGTGTCCTTCATCCTTCGTGCCCTGTCCAAC
TTCATCAAGGACGCCGTGCTCTTCTCCTCAGATGATGTCACCTACTGCGATGCCCACAGGGCGGGCTGCAACCTGGTCATGGTG
CTGTTCCAGTACTGCATCATGGCCAACTACTCCTGGCTGCTGGTGGAAGGCCTCTACCTTCACACACTCCTCGCCATCTCCTTC
TTCTCTGAAAGAAAGTACCTCCAGGGATTTGTGGCATTCGGATGGGGTTCTCCAGCCATTTTTGTTGCTTTGTGGGCTATTGCC
AGACACTTTCTGGAAGATGTTGGCTGTCCCTCCCTTAGGTGCTGGGACATCAATGCCAACGCATCCATCTGGTGGATCATTCGT
GGTCCTGTGATCCTCTCCATCCTGATTAATTTCATCCTTTTCATAAACATTCTAAGAATCCTGATGACAAAACTTACAACCCAA
GAAACAAGAGGAAATGAAGTCAGCCATTATAACCCCCTGGCCAGGTCCACTCTCCTGCTGATCCCCCTCTTTGGCATCCACTAC
ATCGTCTTCCCCTTCTCCCCAGAGGACGCTATGGAGATCCAGCTGTTTTTTGAACTAGCCCTTGCCTCATTCCAGGGACTGGTG
GTGGCCGTCCTCTACTGCTTCCTCAACGGGGAGGTGCAGCTGGAGGTTCAGAAGAAGTGGCACCAATGGCACCTCCGTGAGTTC
CCACTGCACCCCGTGGCCTCCTTCAGCAACAGCACCAAGGCCAGCCACTTCGAGCAGAGCCAGGGCACCTGCAGGACCAGCATC
ATCTGAGAAGCTGGAGCAGG

The nucleic acid sequence of NOV2a maps to chromosome 2q14.1 has 868 of 932 bases (93%) identical to a gb:GENBANK-ID:HSU13989|acc:U13989.1 mRNA from Homo sapiens (Human secretin receptor mRNA, complete cds) (E=2.5e⁻¹⁷⁶).

The NOV2a polypeptide (SEQ ID NO: 10) is 421 amino acid residues in length and is presented using the one-letter amino acid code in Table . The SignalP, Psort and/or Hydropathy results predict that NOV2a is likely to be localized at the plasma membrane with a certainty of 0.6000. In alternative embodiments, a NOV2a polypeptide is located to the Golgi body with a certainty of 0.4000, the endoplasmic reticulum (membrane) with a certainty of 0.3000, or the microbody (peroxisome) with a certainty of 0.3000.

TABLE 1B
Encoded NOV2a Protein Sequence

(SEQ ID NO:10)

TGALPRLCDVLQVLWEEQDQCLQELSREQTGDLGTEQPVPGCEGMWDNISCWPSSVPGRMVEVECPRFLRMLTSRNGSLFRN
CTQDGWSETFPRPNLACGVNVNDSSNEKRHSYLLKLKVMYTVGYSSSLVMLLVALGILCAFRRLHCTRNYIHMHLFVSFILR
ALSNFIKDAVLFSSDDVTYCDAHRAGCKLVMVLFQYCIMANYSWLLVEGLYLHTLLAISFFSERKYLQGFVAFGWGSPAIFV
ALWAIARHFLEDVGCPSLRCWDINANASIWWIIRGPVILSILINFILFINILRILMRKLRTQETRGNEVSHYKRLARSTLLL
IPLFGIHYIVFAFSPEDAMEIQLPFELALGSFQGLVVAVLYCFLNGEVQLEVQKKWQQWHLREFPLHPVASFSNSTKASHLE
QSQGTCRTSII

The NOV2a amino acid sequence to have 416 of 421 amino acid residues (98%) identical to, and 416 of 421 amino acid residues (98%) similar to, the 440 amino acid residue ptnr:SWISSPROT-ACC:P47872 protein from Homo sapiens (Human) (SECRETIN RECEPTOR PRECURSOR (SCT-R)) (E=3.7e⁻²²⁷).

NOV2a is expressed in at least the following tissues: pancreas, lung. This information was derived by determining the tissue sources of the sequences that were included in the invention including but not limited to SeqCalling sources.

Possible small nucleotide polymorphisms (SNPs) found for NOV2a are listed in Tables 2C.

TABLE 2C
SNPs

| | Nucleotide | Base | Amino Acid | Base |
|---|---|---|---|---|
| Variant | Position | Change | Position | Change |
| C110.477 | 1119 | C > T | NA | NA |

NOV2b

A disclosed NOV2b nucleic acid (designated as CuraGen Acc. No. CG56213-02), which includes the 789 nucleotide sequence (SEQ ID NO: 11) shown in Table 2D. An open reading frame for the mature protein was identified with an ATG codon beginning at nucleotides 76-78 and ending with a TGA codon at nucleotides 559-561. The start and stop codons of the open reading frame are highlighted in bold type. Putative untranslated regions are underlined and found upstream from the initiation codon and downstream from the termination codon.

TABLE 2D
NOV2b Nucleotide Sequence

(SEQ ID NO:11)

GGCGCTGAGCTCCCGAGCGGGCAGAGGGCACGGGCAGGCGGACGTCGGGGCGCCCTCGGGGAACGTGCGGGCACCATGCGTCC
CCACCTGTCGCCGCCGCTGCAGCAGCTACTACTGCCGGTGCTGCTCGCCTGCGCCGCGCACTCGCACTCCTACCTGCTGAAGC
TGAAAGTCATGTACACCGTGGGCTACAGCTCCTCCCTGGTCATGCTCCTGGTCGCCCTTGGCATCCTCTGTGCTTTCCGGAGG
CTCCACTGCACTCGCAACTACATCCACATGCACCTGTTCGTGTCCTTCATCCTTCGTGCCCTGTCCAACTTCATCAAGGACGC
CGTGCTCTTCTCCTCAGATGATGTCACCTACTGCGATGCCCACAGGGGACTGGTGGTGGCCGTCCTCTACTGCTTCCTCAACG
GGGACGTGCAGCTGGAGGTTCAGAAGAAGTGGCAGCAATGGCACCTCCGTGAGTTCCCACTGCACCCCGTGGCCTCCTTCAGC
AACACCACCAAGGCCAGCCACTTGGAGCAGAGCCTGGGCACCTGCAGGACCAGCATCATCTGAGAGGCTGGAGCAGGGTCACC
CATGGACAGAGACCAAGAGAGGTCCTGCGAAGGCTGCGCACTGCTGTGGGACAGCCAGTCTTCCCAGCAGACACCCTGTGTCC
TCCTTCAGCTGAAGATGCCCCTCCCCAGGCCTTGGACTCTTCCGAAGGGATGTGAGGCACTGTGGGGCAGGACAAGGGCCTGG
GATTTGGTTCGTTTGCTCTTCTGGGAAGAGAAGTTCAGGGGT

The nucleic acid sequence of NOV2b maps to chromosome 2q14.1 has 472 of 526 bases (89%) identical to a gb:GENBANK-ID:HSU28281|acc:U28281.1 mRNA from Homo sapiens (Human secretin receptor mRNA, complete cds) (E=4.1e⁻¹¹⁸).

The NOV2b polypeptide (SEQ ID NO: 12) is 161 amino acid residues in length and is presented using the one-letter amino acid code in Table 2E. The SignalP, Psort and/or Hydropathy results predict that NOV2b has a signal peptide and is likely to be localized at the plasma membrane with a certainty of 0.4600. In alternative embodiments, a NOV2b polypeptide is located to the microbody (peroxisome) with a certainty of 0.2543, the endoplasmic reticulum (membrane) with a certainty of 0.1000, or the endoplasmic reticulum (lumen) with a certainty of 0.1000. The SignalP predicts a likely cleavage site for a NOV2b peptide between amino acid positions 61 and 62, i.e. at the dash in the sequence LHC-TR.

TABLE 2E
Encoded NOV2b Protein Sequence

(SEQ ID NO:12)

MRPHLSPPLQQLLLFVLLACAAHSHSYLLKLKVMYTVGYSSSLVMLLVALGILCAFRRLHCTRNYIHMHLFVSFILRALSNF
IKDAVLFSSDDVTYCDAHRGLVVAVLYCFLNGEVQLEVQKKWQQWHLREFPLHPVASFSNSTKASHLEQSLGTCRTSII

The NOV2b amino acid sequence has 82 of 92 amino acid residues (89%) identical to, and 84 of 92 amino acid residues (91%) similar to, the 440 amino acid residue ptnr:SWISSPROT-ACC:P47872 protein from Homo sapiens (Human) (SECRETIN RECEPTOR PRECURSOR (SCT-R)) (E=9.6e⁻⁸¹).

NOV2b is expressed in at least the following tissues: pancreas, lung. Expression information was derived from the tissue sources of the sequences that were included in the derivation of the sequence of NOV2b.

NOV2c

A disclosed NOV2c nucleic acid (designated as CuraGen Acc. No. CG56213-03), which includes the 1633 nucleotide sequence (SEQ ID NO: 13) shown in Table 2F. An open reading frame for the mature protein was identified beginning with an ATG codon at nucleotides 109-111 and ending with a TAA codon at nucleotides 979-981. The start and stop codons of the open reading frame are highlighted in bold type. Putative untranslated regions are underlined and found upstream from the initiation codon and downstream from the termination codon.

TABLE 2F
NOV2c Nucleotide Sequence

(SEQ ID NO:13)

ACGAGGCCGGCCGGAGCCCGGGACCCTGCGCGGGGCGCTGAGCTCCCGAGCGGGCAGAGGGCACGGGCAGGCGGACGTCGGGGC
GCCCTCGGGGAACGTGCGGGCACCATGCGTCCCCACCTGTCGCCGCCGCTGCAGCAGCTACTACTGCCGGTGCTGCTCGCCTGC
GCCGCGCACTCGACTGGAGCCCTTCCCCGACTATGTGACGTGCTACAAGTGCTGTGGGAAGAGCAAGACCAGTGCCTGCAGGAA
CTCTCCAGAGAGCAGACAGGAGACCTGGGCACGGAGCAGCCAGTGCCAGGTTGTGAGGGGATGTGGGACAACATAAGCTGCTGG
CCCTCTTCTGTGCCGGGCCGGATGGTGGAGCTGGAATGCCCGAGATTCCTCCGGATGCTCACCAGCACAAATGGTTCCTTGTTC
CGAAACTGCACACACGATGGCTGGTCAGAAACCTTCCCCAGGCCTAATCTGGCCTGTGGCGTTAATGTGAACGACTCTTCCAAC
GAGAAGCGCCACTCCTACCTGCTGAAGCTGAAAGTCATGTACACCGTGGGCTACAGCTCCTCCCTGGTCATGCTCCTGGTCGCC
CTTGGCATCCTCTGTGCTTTCCGGAGGCTCCACTGCACTCGCAACTACATCCACATGCACCTGTTCGTGTCCTTCATCCTTCGT
GCCCTGTCCAACTTCATCAAGGACGCCGTGCTCTTCTCCTCAGATGATGTCACCTACTGCGATCCGCACAGCGCCGCCTGCAAG
CTGGTCATGGTGCTGTTCCAGTACTGCATCATGGCCAACTACTCCTGGCTGCTGGTGGAAGGCCTCTACCTTCACACACTCCTC
GCCATCTCCTTCTTCTCTGAAAGAAAGTACCTCCAGGGATTTGTGGCATTCGGATGGGGTTCTCCACCCATTTTTGTTGCTTTG
TGGGCTATTGCCAGACACTTTCTGGAAGATGTTGCATTAATTTCATCCTTTTCATAAACATTCTAAGAATCCTGATGAGAAAAC
TTAGAACCCAAGAAACAAGAGGAAATGAAGTCAGCCATTATAAGCGCCTGGCCAGGTCCACTCTCCTGCTCATCCCCCTCTTTG
GCATCCACTACATCGTCTTCGCCTTCTCCCCAGAGGACGCTATGGAGATCCAGCTGTTTTTTGAACTAGCCCTTGGCTCATTCC
AGGGACTGGTGGTGACCGTCCTCTACTGCTTCCTCAACGGGGAGGTGCAGCTGGAGGTTCAGAAGAAGTGGCAGCAATGGCACC
TCCGTGAGTTCCCACTGCACCCCGTGGCCTCCTTCAGCAACAGCACCAAGGCCAGCCACTTGGAGCAGAGCCAGGGCACCTGCA
GGACCAGCATCATCTGAGAGGCTGGAGCAGGGTCACCCACGGACAGAGACCAAGAGAGGTCCTGCGAAGGCTGGGCACTGCTGT
GGGACAGCCAGTCTTCCCAGCAGACACCCTGTGTCCTCCTTCAGCTGAACATGCCCCTCCCCAGGCCTTGGACTCTTCCGAAGG
GATGTGAGGCACTGTGGGGCAGGACAAGGGCCTGGGATTTGGTTCGTTTGCTCTTCTGGGAAGAGAAGTTCAGGGGTCCCAGAA
AGGGACAGGGAAATAAATGGTGCCTGGGATGAGATTC

The nucleic acid sequence of NOV2c maps to chromosome 2q14.1 invention has 960 of 961 bases (99%) identical to a gb:GENBANK-ID:HSU28281|acc:U28281.1 mRNA from Homo sapiens (Human secretin receptor mRNA, complete cds) (E=0.0).

The NOV2c polypeptide (SEQ ID NO: 14) is 290 amino acid residues in length and is presented using the one-letter amino acid code in Table 2G. The SignalP, Psort and/or Hydropathy results predict that NOV2c has a signal peptide and is likely to be localized extracellularly at the plasma membrane with a certainty of 0.4600. In alternative embodiments, a NOV2c polypeptide is located to the microbody (peroxisome) with a certainty of 0.1589, the endoplasmic reticulum (membrane) with a certainty of 0.1000, or the enodplasmic reticulum (lumen) with a certainty of 0.1000. The SignalP predicts a likely cleavage site for a NOV7c peptide between amino acid positions 27 and 28 at the dash in the sequence TGA-LP.

TABLE 2G
Encoded NOV2b Protein Sequence

(SEQ ID NO:14)

MRPHLSPPLQQLLLPVLLACAAHSTGALPRLCDVLQVLWEEQDQCLQELSREQTGDLGTEQPVPGCEGMWDNISCW
PSSVPGRMVEVECPRFLRMLTSRNGSLFRNCTQDGWSETFPRPNLACGVNVNDSSNEKRHSYLLKLKVMYTVGYSS
SLVMLLVALGILCAFRRLHCTRNYIHMHLFVSFILRALSNFIKDAVLFSSDDVTYCDPHRAGCKLVMVLFQYCIMA
NYSWLLVEGLYLHTLLAISFFSERKYLQGFVAFGWGSPAIFVALWAIARHFLEDVGLISSFS

The NOV2c amino acid sequence have 283 of 284 amino acid residues (99%) identical to, and 283 of 284 amino acid residues (99%) similar to, the 440 amino acid residue ptnr:SWISSPROT-ACC:P47872 protein from Homo sapiens (Human) (SECRETIN RECEPTOR PRECURSOR (SCT-R)) (E=1.5e⁻¹⁵⁵).

NOV2c is expressed in at least the following tissues:: pancreas, lung. Expression information was derived from the tissue sources of the sequences that were included in the derivation of the sequence of NOV2c.

NOV2a, NOV2 b and NOV2c are very closely homologous as is shown in the amino acid alignment in Table 2H.

[image]

[image]

Homologies to any of the above NOV2 proteins will be shared by the other NOV2 proteins insofar as they are homologous to each other as shown above. Any reference to NOV2 is assumed to refer to both of the NOV2 proteins in general, unless otherwise noted.

NOV2a also has homology to the amino acid sequences shown in the BLASTP data listed in Table 2I.

TABLE 2I
BLAST results for NOV1

| Gene Index/ | | Length | Identity | Positives | |
|---|---|---|---|---|---|
| Identifier | Protein/Organism | (aa) | (%) | (%) | Expect |
| Gi\|2506489\|sp\|P47 | SECRETIN RECEPTOR | 440 | 398/421 | 398/421 | 0.0 |
| 872\|SCRC_HUMAN | PRECURSOR (SCT-R) | | (94%) | (94%) | |
| Gi\|4506825\|ref\|NP | secretin receptor | 440 | 397/421 | 397/421 | 0.0 |
| _002971.1\| | precursor; | | (94%) | (94%) | |
| (NM_002980) | pancreatic | | | | |
| | secretin | | | | |
| | receptor [Homo | | | | |
| | sapiens] | | | | |
| Gi\|763534\|gb\|AAA6 | secretin receptor | 440 | 397/421 | 398/421 | 0.0 |
| 4949.1\| | [Homo sapiens] | | (94%) | (94%) | |
| (U13989) | | | | | |
| Gi\|1085380\|pir\|\|J | secretin receptor - | 440 | 396/421 | 396/421 | 0.0 |
| C2532 | human | | (94%) | (94%) | |
| Gi\|13592081\|ref\|N | secretin receptor | 449 | 331/430 | 362/430 | 0.0 |
| P_112377.1\| | [Rattus | | (76%) | (83%) | |
| (NM_031115) | norvegicus] | | | | |

The homology of these sequences is shown graphically in the ClustalW analysis shown in Table 2J.

[image]

[image]

Tables 2K, 2L and 2M list the domain description from DOMAIN analysis results against NOV2. This indicates that the NOV2 sequence has properties similar to those of other proteins known to contain these domains.

TABLE 2K
Domain Analysis of NOV2

gnl|Pfam|pfam00002, 7tm_2,7 transmembrane receptor (Secretin family)
(SEQ ID NO:69)
Length = 249 residues, 100% aligned
Score = 260 bits (664), Expect = 1e−70

| | | | |
|---|---|---|---|
| Query: | 115 | LLKLKVMYTVGYSSSLVMLLVALGILCAFRRLHCTRNYIHMHLFVSFILRALSNFIKDAV | 174 |
| | | \| \| \|+\|\|\|\|\|\| \|\|\| \|\|+\|+ \| \|\|+\| \|\|\|\|\|\|\|++\|\|+\| \|\|\|\|\|\| \| \|\|\| | |
| Sbjct: | 1 | ALLLSVIYTVGYSLSLVCLLLAIAIFLFFRKLRCTRNYIHLNLFLSLILRALSFLIGDAV | 60 |
| Query: | 175 | LFSSDDVTYCDAHRAGCKLVMVLFQYCIMANYSWLLVEGLYLHTLLAISFFSERKYLQGF | 234 |
| | | \| +\| \|\|\|+\| \| \| +\|\|+ \|+\|\|\|\|\|\|\|+\|\|\| +\|\|\|\|\| \| + | |
| Sbjct: | 61 | LLNSGG--------LGCKVVAVFLHYFFLANFFWMLVEGLYLYTLLVETFFSERLRLLWY | 112 |
| Query: | 235 | VAFGWGSPAIFVALWAIAR-HFLEDVGCPSLRCWDINANASIWWIIROPVILSILINFIL | 293 |
| | | + \|\|\| \|\|+ \| +\|\|+ \| + \|\| \|\| \| \|\|\| +\|\|\|+\| \|\|+\|\|\| | |
| Sbjct: | 113 | LLIGWGVPAVVVGIWALVRPKGYGNEGC----CWLSN-EGGFWWIFKGPVLLIILVNFIF | 167 |
| Query: | 294 | FINILRILMRKLRTQETRGNEVSHYKRLARSTLLLIPLFGIHYIVFAFSPED-AMEIQLF | 352 |
| | | \|\|\|\|\|\|+\|++\|\|\|+ +\| ++ \|++\| +\|\|\|+\|+\|\| \|+ +\|+\| \|+\|\| + + \|+ | |
| Sbjct: | 168 | FINILRVLVQKLRSPQTGkTDL--YRKLVKSTLVLLPLLGVTWILFLFAPESQSSLVFLY | 225 |
| Query: | 353 | FELALGSFQGLVVAVLYCFLNGEV | 376 |
| | | \| \| \|\|\|\| \|\|\|\|\|\|\|\|\|\|\|\| | |
| Sbjct: | 226 | LFLILNSFQGFFVAVLYCFLNGEV | 249 |

TABLE 2L
Domain Analysis of NOV2

gnl|Pfam|pfam02793, HRM, Hormone receptor domain. (SEQ ID NO:70)
Length = 249 residues, 100% aligned
Score = 260 bits (664), Expect = 1e−70

| | | | |
|---|---|---|---|
| Query: | 115 | LLKLKVMYTVGYSSSLVMLLVALGILCAFRRLHCTRNYIHMHLFVSFILRALSNFIKDAV | 174 |
| | | \| \| \|+\|\|\|\|\|\| \|\|\| \|\|+\|+ \| \|\|+\| \|\|\|\|\|\|\|++\|\|+\| \|\|\|\|\|\| \| \|\|\| | |
| Sbjct: | 1 | ALLLSVIYTVGYSLSLVCLLLAIAIFLFFRKLRCTRNYIHLNLFLSLILRALSFLIGDAV | 60 |
| Query: | 175 | LFSSDDVTYCDAHRAGCKLVMVLFQYCIMANYSWLLVEGLYLHTLLAISFFSERKYLQGF | 234 |
| | | \| +\| \|\|\|+\| \| \| +\|\|+ \|+\|\|\|\|\|\|\|+\|\|\| +\|\|\|\|\| \| + | |
| Sbjct: | 61 | LLNSGG--------LGCKVVAVFLUYFPLANFFWMLVEGLYLYTLLVETFFSERLRLLWY | 112 |
| Query: | 235 | VAFGWGSPAIFVALWAIAR-HFLEDVGCPSLRCWDINANASIWWIIRGPVILSILINFIL | 293 |
| | | + \|\|\| \|\|+ \| +\|\|+ \| + \|\| \|\| \| \|\|\| +\|\|\|+\| \|\|+\|\|\| | |
| Sbjct: | 113 | LLIGWGVPAVVVGIWALVRPKGYCNEGC----CWLSN-EGGFWWIFKGPVLLIILVNFIF | 167 |
| Query: | 294 | FINILRILMRKLRTQETRGNEVSHYKRLARSTLLLIPLFGIHYIVFAFSPED-AMEIQLF | 352 |
| | | \|\|\|\|\|\|+\|++\|\|\|+ +\| ++ \|++\| +\|\|\|+\|+\|\| \|+ +\|+\| \|+\|\| + + \|+ | |
| Sbjct: | 168 | FINILRVLVQKLRSPQTGKTDL--YRKLVKSTLVLLPLLGVTWILFLFAPESQSSLVFLY | 225 |
| Query: | 353 | FELALGSFQGLVVAVLYCFLNGEV | 376 |
| | | \| \| \|\|\|\| \|\|\|\|\|\|\|\|\|\|\|\| | |
| Sbjct: | 226 | LFLILNSFQGFFVAVLYCFLNGEV | 249 |

This extracellular domain contains four conserved cysteines that probably for disulphide bridges. The domain is found in a variety of hormone receptors. It may be a ligand binding domain.

TABLE 2M
Domain Analysis of NOV2

gnl|Smart|smart00008, HormR, Domain present in hormone receptors
(SEQ ID NO:71)
Length = 70 residues, 95.7% aligned
Score = 66.6 bits (161), Expect = 3e−12

| | | | |
|---|---|---|---|
| Query: | 41 | GCEGMWDNISCWPSSVPGRMVEVECPRFLRMLTSRNGSLFRNCTQD-GWSETFPRPNLAC | 99 |
| | | \|\| \|\| \| \|\|\| + \|++\|\|\| \|\| + +++ \| \|\|\|\|++ \|\|\| \|\| \| | |
| Sbjct: | 4 | GCPATWDGIICWPQTPAGQLVEVPCPDYFSGFSNKTG-ASRNCTENGGWSPPFPNY-SNC | 61 |
| Query: | 100 | GVNVNDSSN | 108 |
| | | \| + | |
| Sbjct: | 62 | TSNDYNELK | 70 |

Secretin (SCT; OMIM #182099) occupies a unique position in the history of gastrointestinal hormones because it was the first to be discovered, in duodenal mucosa by Bayliss and Starling (1902). This 27-amino acid peptide stimulates the secretion of bicarbonate, enzymes, and potassium ion by the pancreas. Ishihara et al. (1991) isolated a cDNA encoding the rat secretin receptor. The nucleotide sequence showed that the secretin receptor has a calculated molecular weight of 48,696. It contains 7 putative transmembrane segments and belongs to a family of the G protein-coupled receptors, which includes parathyroid hormone receptor (OMIM #168468), glucagon-like receptor (OMIM #138032), and calcitonin receptor (OMIM #114131).

Chow (1995) showed that the secretin receptor cDNA isolated from a pancreatic adenocarcinoma cell-line cDNA library was 1,717 bp long and encoded a 440-amino acid polypeptide. By Northern blot analysis, a 1.8-kb mRNA was detected in human pancreas and intestine, while weak hybridization signals were detected in human colon, kidney, and lung.

NOV2 protein and nucleic acid disclosed herein suggest that it may have important structural and/or physiological functions characteristic of the Secretin receptor precursor family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: developmental diseases, MHCII and III diseases (immune diseases), taste and scent detectability disorders, Burkitt's lymphoma, corticoneurogenic disease, signal transduction pathway disorders, retinal diseases including those involving photoreception, cell growth rate disorders; cell shape disorders, feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and treatment of Albright hereditary ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. dentatorubro-pallidoluysian atrophy(DRPLA) hypophosphatemic rickets, autosomal dominant (2) acrocallosal syndrome and dyskinesias, such as Huntington's disease or Gilles de la Tourette syndrome and/or other pathologies and disorders of the like.. The polypeptides can be used as immunogens to produce antibodies specific for the invention, and as vaccines. They can also be used to screen for potential agonist and antagonist compounds. or example, a cDNA encoding the NOV2 protein may be useful in gene therapy, and the NOV2 protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and treatment of Albright hereditary ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The novel nucleic acid encoding the NOV2 protein, and the NOV2 protein of the invention, or fragments thereof, may further be useful in diagnostic applications, wherein the presence or amount of the nucleic acid or the protein are to be assessed. These materials are further useful in the generation of antibodies that bind immuno specifically to the novel substances of the invention for use in therapeutic or diagnostic methods, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation; colon cancer, colorectal cancer; colorectal cancer; familial nonpolyposis, type 6; esophageal cancer; hepatoblastoma; hypobetalipoproteinemia, familial, 2; lung cancer; metaphyseal chondrodysplasia, Murk Jansen type; ovarian carcinoma, endometrioid type; pilomatricoma; Pseudo-Zellweger syndrome and other diseases, disorders and conditions of the like.

The novel nucleic acid encoding the secretin receptor precursor-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV2 protein has multiple hydrophilic regions, each of which can be used as an immunogen. In one embodiment, a contemplated NOV2 epitope is from about amino acids 10 to 25. In another embodiment, a contemplated NOV2 epitope is from about amino acids 70 to 80. In alternative embodiments, contemplated NOV2 epitopes include from about amino acids 100 to 120, 160 to 170, 230 to 235, 255 to 260, 310 to 320, 370 to 380 and 400 to 405.

NOV3

NOV3 includes two novel B7-H2 like proteins. The disclosed proteins have been named NOV3a and NOV3b.

NOV3a

A disclosed NOV3a nucleic acid (designated as CuraGen Acc. No. CG55790-03), which encodes a novel B7-H2-like protein and includes the 1449 nucleotide sequence (SEQ ID NO: 15) shown in Table 3A. An open reading frame for the mature protein was identified beginning with an ATG codon at nucleotides 2-4 and ending with a TGA codon at nucleotides 908-910. Putative untranslated regions downstream from the termination codon and upstream from the initiation codon are underlined in Table 3A, and the start and stop codons are in bold letters.

TABLE 3K
NOV3a Nucleotide Sequence

(SEQ ID NO:15)

CATGCGGCTGGGCAGTCCTGGACTGCTCTTCCTGCTCTTCAGCAGCCTTCGAGCTGATACTCAGGAGAAGGAAGTC
AGAGCGATGGTAGGCAGCGACGTGGAGCTCAGCTGCGCTTGCCCTGAAGGAAGCCGTTTTGATTTAAATGATGTTT
ACGTATATTGGCAAACCAGTGAGTCGAAAACCGTGGTGACCTACCATATCCCACAGAACAGCTCCTTGGAAAACGT
GGACAGCCGCTACCGGAACCGAGCCCTGATGTCACCGGCCGGCATGCTGCGGGGCCACTTCTCCCTGCGCTTGTTC
AACGTCACCCCCCAGGACGAGCAGAAGTTTCACTGCCTGGTGTTGAGCCAATCCCTGGGATTCCAGGACGTTTTGA
GCGTTGAGGTTACACTGCATGTGGCAGCAAACTTCAGCGTGCCCGTCGTCAGCGCCCCCCACAGCCCCTCCCAGGA
TGAGCTCACCTTCACGTGTACATCCATAAACGGCTACCCCAGGCCCAACGTGTACTGGATCAATAAGACGGACAAC
AGCCTGCTGGACCAGGCTCTGCAGAATGACACCGTCTTCTTGAACATGCGGGGCTTGTATGACGTGGTCAGCGTGC
TGAGGATCGCACGGACCCCCAGCGTGAACATTGGCTGCTGCATAGAGAACGTGCTTCTGCAGCAGAACCTGACTGT
CGGCAGCCAGACAGGAAATGACATCGGAGAGAGAGACAAGATCACAGAGAATCCAGTCAGTACCGGCGAGAAAAAC
GCGGCCACGTGGAGCATCCTGGCTGTCCTGTGCCTGCTTGTGGTCGTGGCGGTGGCCATAGGCTGGGTGTGCAGGG
ACCGATGCCTCCAACACAGCTATGCAGGTGCCTGGGCTGTGAGTCCGGACACAGAGCTCACTGCCCACGTTTGACC
GGAGCTCACCGCCCAGAGCGTGGACAGGGCTTCCATGAGACGCCACCGTGAGAGGCCAGGTGGCAGCTTGAGCATG
GACTCCCAGACTGCAGGGGAGCACTTGGGGCAGCCCCCAGAAGGACCACTGCTGGATCCCAGGGAGAACCTGCTGG
CGTTGGCTGTGATCCTGGAATGAGGCCCTTTCAAAAGCGTCATCCACACCAAAGGCAAATGTCCCCAAGTGAGTGG
GCTCCCCGCTGTCACTGCCAGTCACCCACAGGAAGGGACTGGTGATGGGCTGTCTCTACCCGGAGCGTGCGGGATT
CAGCACCAGGCTCTTCCCAGTACCCCAGACCCACTGTGGGTCTTCCCGTGGGATGCGGGATCCTGAGACCGAAGGG
TGTTTGGTTTAAAAAGAAGACTGGGCGTCCGCTCTTCCAGGACGGCCTCTGTGCTGCTGGGGTCACGCGAGGCTGT
TTGCAGGGGACACGGTCACAGGAGCTCTTCTGCCCTGAACGCTTCCAACCTGCTCCGGCCGGAAGCCACAGGACCC
ACTCA

The nucleic acid sequence of NOV3a maps to chromosome 21 invention has 1448 of 1449 bases (99%) identical to a gb:GENBANK-ID:AF289028|acc:AF289028.1 mRNA from Homo sapiens (Homo sapiens transmembrane protein B7-H2 ICOS ligand mRNA, complete cds) (E=0.0).

The NOV3a polypeptide (SEQ ID NO: 16) is 302 amino acid residues in length and is presented using the one-letter amino acid code in Table 3B. The SignalP, Psort and/or Hydropathy results predict that NOV3a has a signal peptide and is likely to be localized to the plasma membrane with a certainty of 0.4600. In alternative embodiments, a NOV3a polypeptide is located to the lysosome (lumen) with a certainty of 0.2000, the endoplasmic reticulum (membrane) with a certainty of 0.1000, or the endoplasmic reticulum (lumen) with a certainty of 0.1000. The SignalP predicts a likely cleavage site for a NOV3a peptide between amino acid positions 18 and 19, i.e. at the dash in the sequence LRA-DT.

TABLE 3B
Encoded NOV3a Protein Sequence

(SEQ ID NO:16)

MRLGSPGLLFLLFSSLRADTQEKEVRAMVGSDVELSCACPEGSRFDLNDVYVYWQTSESKTVVTYHIPQNSSLENVDSRYR
NRALMSPAGMLRGDFSLRLFNVTPQDEQKFHCLVLSQSLGFQEVLSVEVTLHVAANFSVPVVSAPHSPSQDELTFTCTSIN
GTPRPNVYWINKTDNSLLDQALQNDTVFLNMRGLYDVVSVLRIARTPSVNIGCCIENVLLQQNLTVGSQTGNDIGERDKIT
ENPVSTGEKNAATWSILAVLCLLVVVAVAIGWVCRDRCLQHSYAGAWAVSPETELTGHV

The NOV3a amino acid sequence has 302 of 302 amino acid residues (100%) identical to, and 302 of 302 amino acid residues (100%) similar to, the 302 amino acid residue ptnr:TREMBLNEW-ACC:AAG01176 protein from Homo sapiens (Human) (TRANSMEMBRANE PROTEIN B7-H2 ICOS LIGAND) (E=4.0e161).

NOV3a is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the sequence of NOV3a.

Possible small nucleotide polymorphisms (SNPs) found for NOV3 a are listed in Table 3C and 3D.

TABLE 3C
SNPs

| Consensus | | Base | |
|---|---|---|---|
| Position | Depth | Change | PAF |
| 353 | 10 | G > A | 0.200 |
| 388 | 11 | G > A | 0.273 |

TABLE 3D
SNPs

| | Nucleotide | Base | Amino Acid | |
|---|---|---|---|---|
| Variant | Position | Change | Position | Base Change |
| 13374885 | 260 | T > C | 87 | Ser > Pro |
| 13374884 | 294 | T > C | 98 | Leu > Pro |
| 13374883 | 383 | G > A | 128 | Val > Ile |

NOV3b

A disclosed NOV3b nucleic acid (designated as CuraGen Acc. No. CG55790-04), encoding a novel B7-H2-like protein, which includes the 8250 nucleotide sequence (SEQ ID NO: 17) shown in Table 3E. An open reading frame for the mature protein was identified beginning with an ATG codon at nucleotides 4-6 and ending with a termination codon at nucleotides 1420-1422. The start and stop codons of the open reading frame are highlighted in bold type. Putative untranslated regions are underlined and found upstream from the initiation codon and downstream from the termination codon.

TABLE 3E
NOV3b Nucleotide Sequence

(SEQ ID NO:17)

ACCATGCGGCTGGGCAGTCCTGGACTGCTCTTCCTGCTCTTCAGCAGCCTTCGAGCTGATACTCAGGAGA
AGGAAGTCAGAGCGATGGTAGGCAGCGACGTGGAGCTCAGCTGCGCTTGCCCTGAAGGAAGCCGTTTTGA
TTTAAATGATGTTTACGTATATTGGCAAACCAGTGAGTCGAAAACCGTGGTGACCTACCACATCCCACAG
AACAGCTCCTTGGAAAACGTGGACAGCCGCTACCGGAACCGAGCCCTGATGTCACCGGCCGGCATGCTGC
GGGGCGACTTCTCCCTGCGCTTGTTCAACGTCACCCCCCAGGACGAGCAGAAGTTTCACTGCCTGGTGTT
GAGCCAATCCCTGGGATTCCAGGAGGTTTTGAGCGTTGAGGTTACACTGCATGTGGCAGCAAACTTCAGC
GTGCCCGTCGTCAGCGCCCCCCACAGCCCCTCCCAGGATGAGCTCACCTTCACGTGTACATCCATAAACG
GCTACCCCAGGCCCAACGTGTACTGGATCAATAAGACGGACAACAGCCTGCTGGACCAGGCTCTGCAGAA
TGACACCGTCTTCTTGAACATGCGGGGCTTGTATGACGTGGTCAGCGTGCTGAGGATCGCACGGACCCCC
AGCGTGAACATTGGCTGCTGCATAGAGAACGTGCTTCTGCAGCAGAACCTGACTGTCGGCAGCCAGACAG
GAAATGACATCGGAGAGAGAGACAAGATCACAGAGAATCCAGTCAGTACCGGCGAGAAAAACGCGGCCAC
GTGGAGCATCCTGGCTGTCCTGTGCCTGCTTGTGGTCGTGGCGGTGGCCATAGGCTGGGTGTGCAGGGAC
CGATGCCTCCAACACAGCTATGCAGGTGCCTGGGCTGTGAGTCCGGAGACAGAGCTCACTGGTGAGTTTG
CCGTGGGAAGCAGCAGGTTCTGGGGGGCCCAGGGGAGGCTTGGCTGCCAGCTGTCTTTCAGAGTTTCAAA
AAACTTTCAAAAGGCAAAAGTCCCTTGCCTTGAACAACTGTTGTTCCTGGAGACGCAGCGAAGCCCTCGA
TGGTGCGCATGGCATTTCCTGCAGCCTCCCCTTGGCATGGGATGGCATCCTGGTGTGCACTTTGTCACAC
TGCGATGGGATTTTCCCAACATGCACAGAAGCAGAGAGACGAGTGCTAGACCCCCGCGCTCCCCAGTGCC
CAGCCCCGACCAGGGTGTCCAGGGCGGGTCCAGGCACCGGCGCCCAGCCCCCATGGGGTGTCCGGAGTGG
GTCCAGGCACCGGCGCCCAGCCCCCGTGGGGTGTCCAGGGCGGGTCCAGGCACCGGCGCCCAGCCCCTGT
GGGGTGTCCGGAGTGGGTCCGGGCACCGCCAGCTTCTCTCTGTGGCAGCCACTCCTGCAGCTCTCGTTTG
CCCCTCAGTTCCAGGAGCAACATAGATGTGGATTCCTGTCCAATTTGGGAAAAATGTCCACACACGGTCA
CCCACCTGGCAGGTGCCTCTGGCTGCAAGGGGCGCTGGGCTTCGCAGGCAGGCCAGCCGGGCTCCCCGCC
ATGGGCCAGGATCCCCTCCGAGCCCTGTTTGCCGCCCAGGAGAAGGGGTTCCCCGGGGACAGTGGGCTCA
GGGTGTGCGCAGCCACCATGCTGTGGTGTCACCTGTGGACCCAGGCGAGCTGATGGCCGACCGCAGAAAC
GCACTTCCAAGGCCAGGTCGGCCCATCCAGATGATGCAGGAACACAGCTTGCTAAAAACACGGCCGGCCT
GTTCCCGTCGGAGCCAGTCGAAGTTCCCTGAACAGGCCGCTGTTTCCGAAGCTTTAAACCCTGTGTTTCC
ACCAAGCTGAGTCCTGAGAAAACCGACGTCTGCCTGCAGAAGGGAAAGGGGTGCTTCATGTTCCTCTCTC
TCCTTCATCTCCCTTCCAAGGCCACGTTTGACCGGAGCTCACCGCCCAGAGCGTGGACAGGGCTTCCGTG
ACACGCCACCGTGACAGGCCAGGTGGCAGCTTGAGCATGGACTCCCAGACTGCAGGGGAGCACTTGGGGC
AGCCCCCAGAAGGACCACTGCTGGATCCCAGGGAGAACCTGCTGGCGTTGGCTGTGATCCTGGAATGAGG
CCCTTTCAAAAGCGTCATCCACACCAAAGGCAAATGTCCCCAAGTGAGTGGGCTCCCCGCTGTCACTGCC
AGTCACCCACAGGAAGGGACTGGTGATGGGCTGTCTCTACCCGGAGCGTGCGGGATTCAGCACCAGGCTC
TTCCCAGTACCCCAGACCCACTGTGGGTCTTCCCGTGGGATGCGGGATCCTGAGACCGAAGGGTGTTTGG
TTTAAAAAGAAGACTGGGCGTCCGCTCTTCCAGGACGGCCTCTGTGCTGCTGGGGTCACGCGAGGCTGTT
TGCAGGGGACACGGTCACAGGAGCTCTTCTGCCCTGAACGCTCCCAACCTGCCTCCCGCCCGGAAGCCAC
AGGACCCACTCATGTGTGTGCCCACAAGTGTAGTTAGCCGTCCACACCGAGGAGCCCCCGGAAGTCCCCA
CTGGGCTTCAGTGTCCTCTGCCACATTCCCTGGGAGGAACAATGTCCCTCGGCTGTTCCGGTGAAAAGTT
GAGCCACCTTTGGAAGACGCACGGGTGGAGTTTGCCAGAAGAAAGGCTGTGCCAGGGCCGTGTTTGGCTA
CAGGGGCTGCCGGGGCTCTTGGCTCTGCAGCGAGAAAGACACAGCCCAGCAGGGCTGGAGACGCCCATGT
CCAGCAGGCGCAGGCCTGGCAACACGGTCCCCAGAGTCCTGAGCAGCAGTTAGGTGCATCGAGAGGGTAT
CACCTGGTGGCCACAGTCCCCCTTCTCACCTCAGCAATGATCCCCAAAGTGAGAGGTGGCTCCCCCGGCC
CCCACCACCCTCAGCAGCCCCACCCCACTCAACCCTGAGGGTCCCCAGGGTCCTGATGAAGACCTCCGAC
CCCAGCGCCAGGCTCCTCGGAGCCCAACAGTCCCAAGGGGGCAGGAGACGGGGTGGTCCAGTGCTGAGGG
GTACAGCCCTGGGCCCTGACCAGCCCCGGCACCTGCCATGCTGGTTCCCGGAATGAATCAGCTGCTGACT
GTCTCCAGAAGGGCTGGAAAGGATGCTGCCAGGTGACCCGAGGTGCACTCGCCCCAGGGAGATGGAGTAG
ACAGCCTGGCCTGGCCCTCGGGACACATTGTCTGCCCCGGGGCTATGGGCAAATGCCCCTCCTTCTTACT
TCCCAGAATCCCCTGACATTCCCAGGGTCAGCCAGGACCTGTTACAGCCCTGGTCACTTGGAACTGACAG
CTGTGTGAGGCCTGCACTTCTCAGACCCAGACTTAGAACAAAAGGAGGAGTGAGGACTCAAGGCTACAAT
GAGGTTCCAGTACTTGTTACAAGAAAATTGGTTTTCTGCAAAAAAGTCCCTACCTGAGCCTTTAGGTGAA
TGTGGGATCCACTCCCGCTTTTAACATGAAAGCATTAGAAGATGTGTGGTGTTTATAAAAGAACAGTTGT
CATCACCGGGCATTGATTGGCAGGGACAAGGAGCTGCTTGGGTGTGGAAAGTTGGGGCGTTGGAAAGTGG
GCTGTGGTGCCCATTTGCAGTGACTGTGAAGTGACTCCAGGACGGACCTGCGGGGGCACCCAGAGGTCCT
AAGCCCCAGGACTGAGGGTCGTGCATCACCACTCGGGTGTCCCGGGAGGTGCCCTGGGCCCGGGGACCTC
ACAGGCAGGACGGCGACACTAATGCAGGGAGAGGGAGTCTGGCCCCAGCTTTTCCTATCAGAGGCGATTT
TCCTTCACCAGGGGATGGGCAGGAAAGAGGCAGGGGCCCCAGAAGCTTCTGTCCCTCATGCCTGAGGGCA
CGGGGGACACTTGGAGGCTGCTGTCACCACTGTGCGTCCAAGGCCATGCTCTCTGCGGGTCAGTGCCTGA
GTCTCGCCTCCCTGCTGGTCCCTGAAGCCCCCTCAGAAGCCCTGCCTGTCACGTCGGCATTTGTGAGACC
TACCCTGTAACGCCTGCCCCTCTCAGCCCAACATCAGCTTCCTCTTTCTCCCTTGCTGTAGACAGGCTGG
ATTCCAGTGTTGGGACAGCCATCTCCAGAAACCTGACTTAAGAGAGTAAGATGCAAATCGTGCCTGTATC
CAGTGGCTTTCGTGGGTCCAGGGAGTCTTCGGCACAGCCAGCTCAGCTGTCTGTGGTATGAGCAGGAACA
GGTGCCACTCCTGCTCAGGGGACCCTGCCCTACACCAGGCTGTTCCGTCCCCCTGGAGGACATGGGGCCA
GGTCTGGAGGCATTTTGGGTTGTCACAGCTGGGGGCTGTTCCTCGGCTTCAGCGGCTGGAAGCCTCAGAT
GCTGTTCAACATCTTCTGGACACGGGAGGCCCCGACAGAGAGAAGCGTCCACCCGCAAGTCCACAGTCTG
AGGTCTCCCCTCAGAGACCCTGCCCTGCACACCCACCTCCAGCCAAAGGTCCTGCCTGCCCCAGGGCTCA
GGGGAACCTTGCCGGTCTGTGGAACAGGAGAGGGGACTCTCGCCAGCTGCACCACCCTGCACGTAGTAGG
TGTGCGGTAAACATCCACCAGGGAGGCTCCAGTCAAGGCTGGCAGATGGGGCGGTCCATCCCTAGGGCAG
GTGACAGAAGGGAAAAGGCTGCCTGCTGGCCCCCGAGCCAGGTAGCACATGCTTGTGCCTCAGTTTCCCC
TCCTGTAAAGTGAGGCGCTGGATCCAGGTTCTGTCTACTGGGCTCTGCAGCTTGGACGCTCCTAAGACCA
AGCGACCCACCCTGGGGAGGGCAGCTATGCCTTTGGAATAGCTGTCCAGGCCCGGGTGCCTCCAAGACGG
CCACCACACCCTGCCTGTGCTGCAGGGGTGCAGGGGTAAGGGGCAAGACTCCAGAGCCCTCCTCTCTGCA
TCTCCTTGTCTTCAGTGGCCGGAGGTGAGGCCTGAGCTCAGGGGAGGGGCTTCTGCCACCAACCCTATGG
CGGGGCACAGCACACTTTTCCCAGGGAGGACCCCTGGGCCCCCTGCATTATCCCCAGCGGAGTGTGGGGT
CACCTTCCAAGAGCGACATTGAGAAGCTCCAGCTCTAGGAGTGTGCAGACTCTTAACCAGGCAGGCCCAG
GCCCTGGGGCACACAAAGGCGGGGCCTGCTCTCCCCAGCTGCCCCTCCCAATGGGGGCTGGACTGTCCTA
CCCTCCTCCCTTCTACCTCCCCACTGTCTTCCCTCTCCACTGTCACCACTGCCTCCCTCTTCCACTGTCC
TCCATGCACTGCCCTCCCTCCACCTTCCCCCACCCCCACCACTCCCCATGCTGTCCCCAGGCTCCCCCCG
CTCTCCCCCCTCCCCACTGTCCCCCTCCCCATGCTGTACCCAGCTCACCCCGCTCTCCCCTCTCCCCACT
GTCCCCCCTCCCACTCCCCATGCTGTCCCCAGCTCACCCTACATGGACTTGGCGATGTCCTTCCATGGCT
CACCGGTCTGAATTTCCATGATGAGCCGGGCCTGCAGCTTTGCTCCCCTATCCCTGCCCAGGCTGCAGCT
GTCCATGCAGGGAGCGAGCTCCAGCACCTGCGGAGTCCTTCCGTGGGGGCCTCTCCGTGCCACAGAAGCC
AGGGACCTCAGGTGCCTGTGCATGACACCACCGCCCATCCTCATCCTGAGCCAGCCTCTCAGGATCAGGA
CTTGGTTTGGCGGCGTTAACCTTAGAGCCTGCAAGGGGCTTCCTCCTGGTGGGTCTCGCCGTAGCCTGGG
GAGGCCACAGCTCCAGGCCACTCCAGACCTCCCTTCCTCTGGGCCTTCCATGTGGTGGCAACCACCGCAG
CTGTAAGGGAGGGAAAATGGAGCGTTTGTTCTCGGGCTGGGCTGGGGTCTGGGGGAAGCCATGGGCGTGA
AGACTGGAGTATTATTTGATGGAGAAGCGGCCACTCCTGGAGACCGGCGGCAAACACAGAAGCACAGCGT
GGAAGGTGCTGGTGTCAGCCCACACGGGTGATGGGGTCAGACTCAGGAGTCACACTCAGGAGTCACCAGG
CTCAAAGGGCCCAGGCACCGCAAGTCCTGCTCAGCCCCAGACACAATGCATTCCTGTTGCCCTCGCCCTC
AGCCAGGCCCCACGCAGGCCAGGGAGCACTGGCAAAGCTTGGCAACCCTCTGGGGGCCAGCCTTCATCCA
GGCCGAAGGTGGTCAGTGGCCCACCATGGCCCAGGTAGAAAACTCACGGATTAAGATTTCATGCCCGACT
CCAAAGGCAAGAGACTTTATTATTTTATTTTTTTTGAGCCAGAGTATCGCTCTGTCACCTAGGCTGGAGT
GCAATCTCTGCTCATTGCAACATCTGCCTCCCGAACTCAAGCAATTCTGCCTCAGCCTCCCAAGTAGCTG
GGATTACAGGTGTGCGCCACCATGCCCAGGTAATTGTATTTTTAGTAGAGACAGGGTTTCACCATGTTGG
TCAGGCTGGTTTCAAACTCCTGACCTCAAATGATCTGCCCACCTCGACCTCCCAAAGTGCTGGGATTACA
GGTGCGAGCCACCGCACCTGGCTACCAGACACTTCAGAGTTACAGGTTAGTTTTTCTTTTTCTTTTATTT
TTTTTTTTTTGGCGGAGGTGCAGGGGGAGTTAAACAAACAAACAAAATAAACAGGCCGGGTGCGGTGGCT
CATGCCTGTAATCCCAGCACTTTAGGAGGCCTAGGTGGGTGGATCACGAGATCAGGGGTTCAAGACCAGC
CTGGCCGAGATGGTAAAACCCCGTCTCCACTAAAAATACAAAAATTGGCCAGGCACGGTGGCTCACACCT
GTAATCCCAGTACTTTGGGAGGCTGAGGTGGGCAGATCACCTGAGGTCAGGAGTTCAAGACCAACCTGAC
CAACATGGAGAAACCCCATCTCTACTAAAAATACAAAATTAGCCAGGTGTGGTGGTGCATGCCTGTAATT
CCAGCTACTCGGGAGGCTGAGGCAGGAGAATTGCTTGAACCCAGGAGGCAGAGGTTGCAGTGGGCCAAGA
TGGCGCCATTGCACTCCAGCCTGGGAACAAGAGCGAAACTCTGACTAAAAAAGAAAGAAAGAAAGAAAAA
AATTAGTTGGGCACGGTGGCAGGCGCCTGTAATCCCAGGTACTCAGGAGGCTGAGGCAGGAGAATTGCTT
GAACCCGGGAGGCAGAGGTCGCAGTGAGCCGAGATTGCACCACTGCCCTCCAGCCTGGGTGACAGAGCAA
GACTCCGTCTCAAAAAAAAAAAAAAAAAAAATTGGATACATTGTAATACCTCAAATACTTGTAAGTGAAG
CACCCCAGTTCCCATAGAGCTGCCGCACTCAGAGGCTTCTGTAACCTGCCTGCTCCCAGCATTCTATTTA
GGGTCTGGTATGTCCAGAATTTGCAGACACAGCAATTCCTGCAGCAGCAGTGCACCATGTGGAAGGGGCC
CCATGACCAGCCCACTGTGAGCTCACACGTGATGACTGAGGCTTCTTCACACAGCAGGGCTCTGGGTGTG
ATACCCAGGGCACACGCGTTTGCACACGCACAGGCCACACAAGTTCTCACATGCTCAGCCCCATAAGCCG
TGCTGGACAGGCATGGCCATTTACACCCAGGATCCTGCTGAGAACAGCAACCAACTCACCACCCTCGCAT
CATGATCCTTGCCACACAGGGGCTCTGGTGGCTTTGGTGGCCTGGGCTGTGGCTCTGCTGCCAGCCACCT
TGAGTGAAGATCCGGGTTCTCTGGGTGCTACTCAGCTGCTATGTGGGGAGCTGCCCCCTGGGGTGATGAG
GGCCCTTCCCAACCCGCCCTCAGCCCTTGGACAGCCAGGATCACCCGGGGCTGTCTGCATACAGACTTCT
CAGGGGAGTTCTCAGCTTGGACCCTTATCTCCCCAGAATCCTGGAACCTGCTCCTTCTGCTCTCGTGACT
GACTGTGTTCTCTATGCAACTTCCAATAAAACCTCTTCATTTGAAAGGAAAAAAGTCTGCATTATCTGTT
TAGGAAGGGAGAGAGTTCATATTGCAATCTTTTTTTTTTTAATAAAAATAATCTCAGCCTGGGCAACATG
GTGAGACCCCATCTCTGTAAAACATTTTTAAAAAATTAGCCGGGTATGGTGGCGCACACTTGTAGTCCCA
GCTACTCAGGAGGCTGAAGCGGGAGGATCCATTGAACCTGAGAAGTCGAAGCTGCAGTGAGCTGTGATTG
TGCCACTGTACTCCAGCCTGGACAACAGAGTGAGACGCCGTCTCAAATAATAAATACAT

The NOV3polypeptide (SEQ ID NO: 18) is 473 amino acid residues in length and is presented using the one-letter amino acid code in Table 3F.

TABLE 3F
Encoded NOV3b Protein Sequence

(SEQ ID NO:18)

MRLGSPGLLFLLFSSLRADTQEKEVRAMVGSDVELSCACPEGSRFDLNDVYVYWQTSESKTVVTYHIPQNSSLENV
DSRYRNRALMSPAGMLRGDFSLRLFNVTPQDEQKFHCLVLSQSLGFQEVLSVEVTLHVAANFSVPVVSAPHSPSQD
ELTFTCTSINGYPRPNVYWINKTDNSLLDQALQNDTVFLNMRGLYDVVSVLRIARTPSVNIGCCIENVLLQQNLTV
GSQTGNDIGERDKITENPVSTGEKNAATWSILAVLCLLVVVAVAIGWVCRDRCLQHSYAGAWAVSPETELTGEFAV
GSSRFWGAQGRLCCQLSFRVSKNFQKAKVPCLEQLLFLETQRSPRWCAWHFLQPPLGMGWHPGVHFVTLRNDFPNM
HRSRETSARPPRSPVPSPDQGVQGGSRHRRPAPMGCPEWVQAFAPSPRGVSRAGPGTGAQPLWGVRSGSGHRQLLS
VAATPAALVCPSVPGAT

NOV3a and NOV3b are very closely homologous as is shown in the amino acid alignment in Table 3G.

[image]

[image]

Homologies to any of the above NOV3 proteins will be shared by the other NOV3 proteins insofar as they are homologous to each other as shown above. Any reference to NOV3 is assumed to refer to both of the NOV3 proteins in general, unless otherwise noted.

NOV3a also has homology to the amino acid sequences shown in the BLASTP data listed in Table 3H.

TABLE 3H
BLAST results for NOV3

| Gene Index/ | Protein/ | Length | Identity | Positives | |
|---|---|---|---|---|---|
| Identifier | Organism | (aa) | (%) | (%) | Expect |
| sp\|O75144\| | HYPOTHETICAL | 558 | 259/284 | 259/284 | e−152 |
| Y653_HUMAN | PROTEIN KIAA0653 | | (91%) | (91%) | |
| | PROTEIN | | | | |
| ref\|XP_036027.2\| | KIAA0653 | 302 | 261/286 | 261/286 | e−149 |
| (XM_036027) | protein, B7-like | | (91%) | (91%) | |
| | protein [Homo | | | | |
| | sapiens] | | | | |
| gb\|AAF34739.1\|AF199 | B7-like protein | 309 | 258/283 | 258/283 | e−147 |
| 028_1 (AF199028) | [Homo sapiens] | | (91%) | (91%) | |
| gb\|AAK77544.1\|AF394 | B7-like protein | 347 | 112/234 | 143/234 | 1e−49 |
| 451_1 (AF394451) | GL50-B [Mus | | (47%) | (60%) | |
| | musculus] | | | | |
| ref\|NP_056605.1\| | icos ligand [Mus | 322 | 112/234 | 143/234 | 2e−49 |
| (NM_015790) | musculus] | | (47%) | (60%) | |

The homology of these sequences is shown graphically in the ClustalW analysis shown in Table 3I.

[image]

[image]

[image]

Table 3J lists the domain description from DOMAIN analysis results against NOV3. This indicates that the NOV3 sequence has properties similar to those of other proteins known to contain these domains.

TABLE 3J
Domain Analysis of NOV3

gnl|Smart|smart00406, IGv, Immunoglobulin (SEQ ID NO:77)
Length = 80 residues, 100% aligned
Score = 35.4 bits (107), Expect = 0.005

| | | | |
|---|---|---|---|
| Query: | 32 | DVELSCACPEGSRFDLNDVYVYWQTSESKTVVTYHIPQNSSLENVDSRYRNRALMSPAGM | 91 |
| | | \| \|\|\| + \| \| + \|\| \| + + \| + ++ \|+ \| +\| | |
| Sbjct: | 1 | SVTLSC---KASGFTFSSYYVSWVRQPPGKGLEWLGYIGSDVSYSEASYKGRVTISKD-N | 56 |
| Query: | 92 | LRGDFSLRLFNVTPQDEQKFHCLV | 115 |
| | | + \| \|\| + \|+ +\| ++\| \| | |
| Sbjct: | 57 | SKNDVSLTISNLRVEDTGTYYCAV | 80 |

Costimulatory interactions between the B7 family ligands and their receptors play critical roles in the growth, differentiation, and death of T cells. Engagement of the T-cell costimulator CD28 by either specific antibodies or its natural ligands B7-1 and B7-2 increases antigen-specific proliferation of CD4⁺ T cells, enhances production of cytokines, induces maturation of CD8⁺ effector T cells and promotes T-cell survival. Signaling through homologous CTLA-4 receptor of B7-1 and B7-2 on activated T cells, however, is thought to deliver a negative signal that inhibits T-cell proliferation, interleukin (IL)-2 production, and cell cycle progression. Although B7-1 and B7-2 share only ˜20% homology in their amino acids, they have similar tertiary structures and costimulatory functions. Recent studies indicate that other members of the B7-CD28 family may also participate in the regulation of cellular and humoral immune responses. One of the new members is an inducible costimulator (ICOS), a CD28-like receptor. An F44 monoclonal antibody (mAb) against human ICOS costimulates T-cell growth and increases secretion of several cytokines including IL-10, interferon-, and IL-4, but not IL-2 in the presence of optimal doses of anti-CD3 antibody.

Another new B7 family member is mouse B7h/B7RP-1. B7h/B7RP-1 does not bind to CD28 and CTLA-4 and can costimulate T-cell growth in the presence of antigenic signals. It has been shown that surface expression of B7h/B7RP-1 is up-regulated by tumor necrosis factor-in the 3T3 fibroblast line and the increase of B7h/B7RP-1 messenger RNA (mRNA) is also observed in nonlymphoid tissues exposed to lipopolysaccharide (LPS). It has been demonstrated that B7h/B7RP-1 is a ligand for mouse CRP-1, a mouse homologue of human ICOS. Expression of a B7RP-1 fusion protein in transgenic mice leads to hyperplasia in several lymphoid organs and treatment of mice with B7h/B7RP-1 fusion protein enhanced oxazolone-induced contact hypersensitivity. A new member of the human B7 family, B7-H1, has recently been reported. B7-H1 shares ˜20% identical amino acid sequence with B7-1 and B7-2 in the Ig V- and Ig C-like extracellular domains but differs more profoundly from B7-1 and B7-2 in the cytoplasmic domain. It is unlikely that B7-H1 is a human homologue of mouse B7h/B7RP-1 because identity of amino acids between them is less than 30%. Furthermore, B7-H1 does not bind to CD28, CTLA-4, and ICOS. Surface expression of B7-H1 can be detected in the majority of activated CD14⁺ macrophages and a fraction of activated T cells.

B7-H1 costimulates T-cell responses in the presence of suboptimal doses of anti-CD3 mAb, enhances allogeneic mixed lymphocyte response, and preferentially induces IL-10 secretion from T cells. By searching molecules sharing homologies with the Ig V and Ig C domains of B7-1, B7-2, and B7-H1 in the NCBI database followed by subsequent cloning and sequencing, a new B7-like gene designated B7-H2 (B7omologue 2) was identified. In addition to an overall structure similarity to B7-1, B7-2, and B7-H1, B7-H2 binds ICOS and costimulates the proliferation and cytokine production of human T cells. Cell surface expression of B7-H2 protein is detected in monocyte-derived immature dendritic cells. Soluble B7-H2 and immunoglobulin (Ig) fusion protein, B7-H2Ig, binds activated but not resting T cells and the binding is abrogated by inducible costimulator Ig (ICOSIg), but not CTLA4Ig. In addition, ICOSIg stains Chinese hamster ovary cells transfected with B7-H2 gene. By suboptimal cross-linking of CD3, costimulation of T-cell proliferation by B7-H2Ig is dose-dependent and correlates with secretion of interleukin (IL)-2, whereas optimal CD3 ligation preferentially stimulates IL-10 production. The results indicate that B7-H2 is a putative ligand for the ICOS T-cell molecule. (Blood. 2000;96:2808-2813) PMID: 11023515, UI: 20477846

The T cell-specific cell surface receptors CD28 and CTLA4 are important regulators of the immune system. CD28 potently enhances those T-cell functions essential for an effective antigen-specific immune response, and CTLA4 counterbalances the CD28-mediated signals and thus prevents an otherwise fatal overstimulation of the lymphoid system. By generating monoclonal antibodies against activated human T cells, another member of this family of molecules, ‘inducible costimulator,’ symbolized ICOS has been identified. The ICOS-specific monoclonal antibody did not react with resting human peripheral blood T cells, but stained CD4+ and CD8+ T lymphocytes that had been activated by stimulation of the T-cell antigen receptor complex. Immunoprecipitations defined the ICOS antigen as a disulfide-linked dimer with an apparent relative molecular mass of 55 to 60 kD. Protein purification by SDS-PAGE indicated that ICOS is expressed on the cell surface as a homodimeric protein, with the 2 chains differing only in their posttranslational modification. The full-length ICOS cDNA of 2,641 basepairs was cloned from a MOLT-4V T lymphoblast cDNA library. Northern analysis revealed a single ICOS mRNA species of approximately 2.8 kb in length in activated human T cells. The open reading frame of ICOS mRNA encodes a protein of 199 amino acids. The ICOS amino acid sequence shares 24% and 17% identity, respectively, with CD28 and CTLA4. The predicted mature ICOS is a type I transmembrane molecule that consists of a single immunoglobulin V-ike domain, stabilized by conserved cysteine residues at positions 42 and 109; a transmembrane region of approximately 23 amino acids; and a cytoplasmic tail of 35 amino acids. It shows close structural resemblance to CD28 and CTLA4. The cysteine residue located at position 141 of CD28, also found in CTLA4, is apparently involved in forming the disulfide bridge between the homodimeric chains of these proteins, and is also found in ICOS at position 136. ICOS matches CD28 in potency and enhances all basic T-cell responses to a foreign antigen, namely proliferation, secretion of lymphokines, upregulation of molecules that mediate cell-cell interaction, and effective help for antibody secretion by B cells. Unlike the constitutively expressed CD28, ICOS has to be de novo induced on the T-cell surface and does not upregulate the production of interleukin-2 (IL2), but superinduces the synthesis of interleukin-10 (IL10), a B-cell differentiation factor. In vivo, ICOS is highly expressed on tonsillar T cells, which are closely associated with B cells in the apical light zone of germinal centers, the site of terminal B-cell maturation

Icos-deficient mice have been generated and it has been determined that the absence of Icos did not impair T-cell development. However, T-cell activation in terms of proliferation and IL2 production was impaired. Differentiated Icos −/− cells were able to produce IFNG but not IL4 or IL2. In vivo immunization also revealed a defect in IL2 and IL4 production and a reduction in serum IgG1 and IgE. Using allergy models, it has been found that Icos was not required for Th2 cell differentiation, but rather it regulated IL4 and IL13 production. Using the experimental autoimmune encephalitis (EAE) model for multiple sclerosis, it has been found that Icos −/− mice developed greatly enhanced disease compared with wildtype mice, even with a genetic background otherwise associated with resistance to EAE. Splenocytes from the knockout and wildtype mice produced undetectable levels of IL4 and similar levels of IL10 and IFNG; however, cells from the Icos −/− mice produced no IL13, whereas wildtype mice made abundant amounts. It has been concluded that ICOS may have an important negative regulatory role, through the induction of IL13, in protection against inflammatory diseases.

It has been found that Icos-deficient mice had similar basal levels of IgM, slightly elevated IgG3, and reduced IgG1, IgG2a, and IgE compared to wildtype mice. Immunized knockout and wildtype mice, except in the presence of the highly inflammatory complete Freund's adjuvant, also had similar levels of IgM-specific antibody but reduced IgG1- and IgG2a-specific antibody and reduced germinal center formation. Class switching from IgM to IgG was restored in Icos −/− mice by stimulation of CD40.

It has been found that reduced T-cell proliferation in cells from Icos-deficient mice was associated with a marked decrease in expression of CD40LG, CD25, and CD69. B-cell activation and T cell-independent antibody responses were unimpaired in Icos knockout mice. It has been found that only basal levels of IgG1 were significantly reduced in Icos −/− mice; however, they concurred that serum IgG1 and IgG2a levels were reduced, and IgE levels were undetectable after immunization. ELISA assays showed that this class-switching impairment was associated with reduced IL4 production but not with IFNG production. Immunohistochemistry analysis determined that germinal center formation was also reduced in Icos knockout mice, as it is in mice deficient in Cd401g or Cd28.

The protein similarity information, expression pattern, cellular localization, and map location for the NOV3 protein and nucleic acid disclosed herein suggest that it may have important structural and/or physiological functions characteristic of the Immunoglobulin domain-containing proteins family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention may have efficacy for the treatment of patients suffering from brain disorders including epilepsy, eating disorders, schizophrenia, ADD, and cancer; heart disease; inflammation and autoimmune disorders including Crohn's disease, IBD, allergies, rheumatoid and osteoarthritis, inflammatory skin disorders, allergies, blood disorders; psoriasis colon cancer, leukemia AIDS; thalamus disorders; metabolic disorders including diabetes and obesity; lung diseases such as asthma, emphysema, cystic fibrosis, and cancer; pancreatic disorders including pancreatic insufficiency and cancer; and prostate disorders including prostate cancer, as well as other diseases, disorders and conditions.

The novel nucleic acid encoding the B7-H2-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV3 protein has multiple hydrophilic regions, each of which can be used as an immunogen. In one embodiment, a contemplated NOV3 epitope is from about amino acids 20 to 25. In another embodiment, a contemplated NOV3 epitope is from about amino acids 40 to 42. In other specific embodiments, contemplated NOV3 epitopes are from about amino acids 48 to 55, 60 to 75, 90 to 120, 145 to 180, 230 to 250 and 270 to 290.

NOV4

NOV4 includes two novel B7-H1-like proteins. The disclosed proteins have been named NOV4a and NOV4b.

NOV4a

A disclosed NOV4a nucleic acid (designated as CG56 110_{—}01), encodes a novel B7-H1-like protein and includes the 4582 nucleotide sequence (SEQ ID NO: 19) shown in Table 4A. An open reading frame for the mature protein was identified beginning with an ATG codon at nucleotides 10-12 and ending with a TAA codon at nucleotides 887-889. Putative untranslated regions downstream from the termination codon and upstream from the initiation codon are underlined in Table 4A, and the start and stop codons are in bold letters.

TABLE 4A

NOV4a Nucleotide Sequence

(SEQ ID NO:19)

TCCAGAAAGATGAGGATATTTGCTGTCTTTATATTCATGACCTACTGGCATTTGCTGAACGCATTTACTGTCACGG
TTCCCAAGGACCTATATGTGGTAGAGTATGGTAGCAATATGACAATTGAATGCAAATTCCCAGTAGAAAAACAATT
AGACCTGGCTGCACTAATTGTCTATTGGGAAATGGAGGATAAGAACATTATTCAATTTGTGCATGGAGAGGAAGAC
CTGAAGGTTCAGCATAGTAGCTACAGACAGAGGGCCCGGCTGTTGAAGGACCAGCTCTCCCTGGGAAATGCTGCAC
TTCAGATCACAGATGTGAAATTGCAGGATGCAGGGGTGTACCGCTGCATGATCAGCTATGGTGGTGCCGACTACAA
GCGAATTACTGTGAAAGTCAATGCCCCATACAACAAAATCAACCAAAGAATTTTGGTTGTGGATCCAGTCACCTCT
GAACATGAACTGACATGTCAGGCTGAGGGCTACCCCAAGGCCGAAGTCATCTGGACAAGCAGTGACCATCAAGTCC
TGAGTGGTAAGACCACCACCACCAATTCCAAGAGAGAGGAGAAGCTTTTCAATGTGACCAGCACACTGAGAATCAA
CACAACAACTAATGAGATTTTCTACTGCACTTTTAGGAGATTAGATCCTGAGGAAAACCATACAGCTGAATTGGTC
ATCCCAGAACTACCTCTGGCACATCCTCCAAATGAAAGGACTCACTTGGTAATTCTGGGAGCCATCTTATTATGCC
TTGGTGTAGCACTGACATTCATCTTCCGTTTAAGACAAGGGAGAATGATGGATGTGAAAAAATGTGGCATCCAAGA
TACAAACTCAAAGAAGCAAAGTGGTAAGAATATCAGAAGGAATTGGGAAGTAAAAGTCAAACGAAACAAAAAGCTA
AAGCAATAACAAAGAGAAATCCATCAGTCATAATCTCCTCTCCTTTTAAAGAATGCTGGTTCCCCTTTGCCTCACA
GCTAACACAAGAACTCCTCCACCGTCTGAGGAGGTTTAGGAGCAGGGAAGGGGAAGGAGTCAGCTTCATTTGCTAA
TCTTCTGTTGCCCTGCACCCTAGCAGCTCCTTGCAGCAGGGGACAAGGATGACTTAGGTGGATGCATAATTAATTG
ATTCTAAAATATTGTGTGTCAGTATTGTAATACTATGTTAATTGCACCATGCACGGTATCTCATTTAATCCCCCAC
CCCTTGCCATTACCAAAGAGAGAGAGAGAGAGAGAGAGAGAAATACTAGAATTTATCCTCATTTTACAGTAGAGAA
AACAGAGGGTCAAGAAGATAATGTAAAGTGCCCAAGAACACACAGCTGATCACAAAAATCAAGCTTGGGGGCCATT
AGCCTAACCACAGACCCTTACTCTTAACCCATCTGCTTCAATCCATTTTGCTACAAATGTTTACATTTATAAGCAG
GGCAGAAAAACCTCATCCAGCTTATTGAACTAACAACAAAGTTATATTAACGTTTCTAATTTTTTTAATGTAGTTA
GAAACCAAACTTAACAATGAGCCCAAGTTTAAAGCAGTCTAATTAACCTGGACAAGCTCAGGCAAGTTTCATTCTG
TGGCCCATAGCATCATCTGTGTTGTAAAGCTAAGTAGCAAATGTTGTTTGGGTCATGCTGGGGGACAAGCCATCCC
AATTTGCTCAGGACTGAGGGGTTTTCCAGGATATCATGTAAGGATAATTGGGTACAAATATAACCTGCTGCTTTCT
CTCATTTCAAATTTATCATTTATCATATCAGCAACTATGAGTTATGTTTTTTATTAGATTTCTTGTTACTTTTTCC
CCAGACCACTTCCCATGAAATTAATATACTATTATCACTCTCCAGATACACATTTGGAGGAGACGTAATCCAGCAT
TGGAACTTCTGATCTTCAAGCAGGGATTCTCAACCTGTGGTTTAGGGGTTCATCGGGGCTGAGCGTGACAAGAGGA
AGGAATGGGCCCGTCGGATGCAGGCAATGTGGGACTTAAAAGGCCCAAGCACTGAAAATGGAACCTGGCGAAAGCA
GAGGAGGAGAATGAAGAAAGATGGAGTCAAACAGGGAGCCTGGAGGGAGACCTTGATACTTTCAPATGCCTGAGGG
GCTCATCGACGCCTGTGACAGGGAGAAAGGATACTTCTGAACAAGGAGCCTCCAAGCAAATCATCCATTGCTCATC
CTAGGAAGACGGGTTGAGAATCCCTAATTTGAGGGTCAGITCCTGCAGAAGTGCCCTTTGCCTCCACTCAATGCCT
CAATTTGTTTTCTGCATGACTGAGAGTCTCAGTGTTGGAACGGGACAGTATTTATGTATGAGTTTTTCCTATTTAT
TTTGAGTCTGTGAGGTCTTCTTGTCATGTGAGTGTGGTTGTGAATGATTTCTTTTGAAGATATATTGTAGTAGATG
TTACAATTTTGTCGCCAAACTAAACTTGCTGCTTAATGATTTGCTCACATCTAGTAAAACATGGAGTATTTGTAAG
GTGCTTGGTCTCCTCTATAACTACAAGTATACATTGGAAGCATAAAGATCAAACCGTTGGTTGCATAGGATGTCAC
CTTTATTTAACCCATTAATACTCTGGTTGACCTAATCTTATTCTCAGACCTCAAGTGTCTGTGCAGTATCTGTTCC
ATTTAAATATCAGCTTTACAATTATGTGGTAGCCTACACACATAATCTCATTTCATCGCTGTAACCACCCTGTTGT
GATAACCACTATTATTTTACCCATCGTACAGCTGAGGAAGCAAACAGATTAAGTAACTTGCCCAAACCAGTAAATA
GCAGACCTCAGACTGCCACCCACTGTCCTTTTATAATACAATTTACAGCTATATTTTACTTTAAGCAATTCTTTTA
TTCAAAAACCATTTATTAAGTGCCCTTGCAATATCAATCGCTGTGCCAGGCATTGAATCTACAGATGTGAGCAAGA
CAAAGTACCTGTCCTCAAGGAGCTCATAGTATAATGAGGAGATTAACAAGAAAATGTATTATTACAATTTAGTCCA
GTGTCATAGCATAAGGATGATGCGAGGGGAAAACCCGAGCAGTGTTGCCAAGAGGAGGAAATAGGCCAATGTGGTC
TGGGACGGTTGGATATACTTAAACATCTTAATAATCAGAGTAATTTTCATTTACAAAGAGAGGTCGOTACTTAAAA
TAACCCTGAAAAATAACACTGGAATTCCTTTTCTAGCATTATATTTATTCCTGATTTGCCTTTGCCATATAATCTA
ATGCTTGTTTATATAGTGTCTGGTATTGTTTAACAGTTCTGTCTTTTCTATTTAAATGCCACTAAATTTTAAATTC
ATACCTTTCCATGATTCAAAATTCAAAAGATCCCATGGGAGATGGTTGGAAAATCTCCACTTCATCCTCCAAGCCA
TTCAAGTTTCCTTTCCAGAAGCAACTGCTACTGCCTTTCATTCATATGTTCTTCTAAAGATAGTCTACATTTGGAA
ATGTATGTTAAAAGCACGTATTTTTAAAATTTTTTTCCTAAATAGTAACACATTGTATGTCTGCTGTGTACTTTGC
TATTTTTATTTATTTTAGTGTTTCTTATATAGCAGATGGAATGAATTTGAAGTTCCCAGGGCTGAGGATCCATGCC
TTCTTTGTTTCTAAGTTATCTTTCCCATAGCTTTTCATTATCTTTCATATGATCCAGTATATGTTAAATATGTCCT
ACATATACATTTAGACAACCACCATTTGTTAAGTATTTGCTCTAGGACAGAGTTTGGATTTGTTTATGTTTGCTCA
AAAGGAGACCCATGGGCTCTCCAGGGTGCACTGAGTCAATCTAGTCCTAAAAAGCAATCTTATTATTAACTCTGTA
TGACAGAATCATGTCTGGAACTTTTGTTTTCTGCTTTCTGTCAAGTATAAACTTCACTTTGATGCTGTACTTGCAA
AATCACATTTTCTTTCTGGAAATTCCGGCAGTGTACCTTGACTGCTAGCTACCCTGTGCCAGAAAAGCCTCATTCG
TTGTGCTTGAACCCTTGAATGCCACCAGCTGTCATCACTACACAGCCCTCCTAAGAGGCTTCCTGGAGGTTTCGAG
ATTCAGATGCCCTGGGAGATCCCAGAGTTTCCTTTCCCTCTTGGCCATATTCTGGTGTCAATGACAAGGAGTACCT
TGGCTTTGCCACATGTCAAGGCTGAAGAAACAGTGTCTCCAACAGAGCTCCTTGTGTTATCTGTTTGTACATGTGC
ATTTGTACAGTAATTGGTGTGACAGTGTTCTTTGTGTGAATTACAGGCAAGAATTGTGGCTGAGCAAGGCACATAG
TCTACTCAGTCTATTCCTAAGTCCTAACTCCTCCTTGTGGTGTTGGATTTGTAAGGCACTTTATCCCTTTTGTCTC
ATGTTTCATCGTAAATGGCATAGGCAGAGATGATACCTAATTCTGCATTTGATTGTCACTTTTTGTACCTGCATTA
ATTTAATAAAATATTCTTATTTATTTTGTTACTTGGTACACCAGCATGTCCATTTTCTTGTTTATTTTGTCTTTAA
TAAAATGTTCAGTTTAACATCC

The nucleic acid sequence of NOV4a maps to chromosome 9 has 672 of 873 bases (76%) identical to a gb:GENBANK-ID:AF317088|acc:AF317088.1 mRNA from Mus musculus (Mus musculus B7-H1 protein mRNA, complete cds) (E=8.5e⁻¹⁰⁶).

The NOV4a polypeptide (SEQ ID NO: 20) is 290 amino acid residues in length and is presented using the one-letter amino acid code in Table 4B. The SignalP, Psort and/or Hydropathy results predict that NOV4a has a signal peptide and is likely to be localized to the plasma membrane with a certainty of 0.4600. In alternative embodiments, a NOV4a polypeptide is located to the endoplasmic reticulum (membrane) with a certainty of 0.1000, the endoplasmic reticulum (lumen) with a certainty of 0.1000, or outside of the cell with a certainty of 0.1000. The SignalP predicts a likely cleavage site for a NOV4a peptide between amino acid positions 18 and 19, i.e. at the dash in the sequence LNA-FT.

TABLE 4B

Encoded NOV4a Protein Sequence

(SEQ ID NO:20)

MRIFAVFIFMTYWHLLNAFTVTVPKDLYVVEYGSNMTIECKFPVEKQLDLAALIVYWEMEDKNIIQFVHGEEDLKVQHSSY
RQRARLLKDQLSLGNAALQITDVKLQDAGVYRCMISYGGADYKRITVKVNAPYNKINQRILVVDPVTSEHELTCQAEGYPK
AEVIWTSSDUQVLSGKTTTTNSKREEKLFNVTSTLRINTTTNEIFYCTFRRLDPEENNTAELVTPELPLAHPPNERTHLVI
LGAILLCLGVALTFIFRLRKGRMMDVKKCGIQDTNSKKQSDTHLEET

The NOV4a amino acid sequence has 202 of 290 amino acid residues (69%) identical to, and 236 of 290 amino acid residues (81%) similar to, the 290 amino acid residue ptnr:TREMBLNEW-ACC:AAG18509 protein from Mus musculus (Mouse) (PD-1-LIGAND PRECURSOR) (E=3.0e⁻¹⁰⁶).

NOV4a is expressed in at least the following tissues LPS treated dendritic cells, LPS treated monocytes and macrophages, brain, cervix, ovary, pituitary gland, placenta, uterus, whole organism. This information was derived by determining the tissue sources of the sequences that were included in the invention including but not limited to SeqCalling sources, Public EST sources, Literature sources, and/or RACE sources.

Possible small nucleotide polymorphisms (SNPs) found for NOV4a are listed in Tables 4C.

TABLE 4C
SNPs

| | Nucleotide | Base | Amino Acid | |
|---|---|---|---|---|
| Variant | Position | Change | Position | Base Change |
| 13376571 | 109 | A > G | 37 | Thr > Ala |
| 13376572 | 242 | A > G | 81 | Tyr > Cys |
| 13374882 | 263 | T > C | 88 | Leu > Ser |
| 13376573 | 346 | A > G | 116 | Ile > Val |
| 13376574 | 578 | T > C | 193 | Val > Ala |

NOV4b

A disclosed NOV4b nucleic acid(designated as CG56110-04), which is a splice variant of NOV4a, includes the 745 nucleotide sequence (SEQ ID NO: 21) shown in Table 4D. An open reading frame for the mature protein was identified beginning with an ATG codon at nucleotides 1-3 and ending with a TAG codon at nucleotides 535-537. The start and stop condons of the open reading frame are highlighted in bold type. Putative untranslated regions are underlined and found upstream from the initiation codon and downstream from the termination codon.

TABLE 4D

NOV4b Nucleotide Sequence

(SEQ ID NO:21)

ATGAGGATATTTGCTGTCTTTATATTCATGACCTACTGGCATTTGCTGAACGCATTTACTGTCACGGTTCCCAAGGAC
CTATATGTGGTAGAGTATGGTAGCAATATGACAATTGAATGCAAATTCCCAGTAGAAAAACAATTAGACCTGGCTGCA
CTAATTGTCTATTGGGAAATGGAGGATAAGAACATTATTCAATTTGTGCATGGAGAGGAAGACCTGAAGGTTCAGCAT
AGTAGCTACAGACAGAGGGCCCGGCTGTTGAAGGACCAGCTCTCCCTGGGAAATGCTGCACTTCAGATCACAGATGTG
AAATTGCAGGATGCAGCGGTGTACCGCTGCATGATCAGCTATGGTGGTGCCGACTACAAGCGAATTACTGTCAAAGTC
AATGCCCCATACAACAAAATCAACCAAAGAATTTTGGTTGTGGATCCAGTCACCTCTGAACATGAACTGACATGTCAG
GCTGAGGGCTACCCCAAGGCCGAAGTCATCTGGACAAGCAGTGACCATCAAGTCCTGAGTCGAGATTAGATCCTGAGG
AAAACCATACAGCTGAATTGGTCATCCCAGAACTACCTCTGGCACATCCTCCAAATGAAAGGACTCACTTGGTAATTC
TGGGAGCCATCTTATTATGCCTTGGTGTAGCACTGACATTCATCTTCCGTTTAAGAAAAGGGAGAATGATGGATGTGA
AAAAATGTGGCATCCAAGATACAAACTCAAAGAAGCAAAGTGG

The nucleic acid sequence of NOV4b maps to chromosome 9 and has 530 of 530 bases (100%) identical to a gb:GENBANK-ID:AF233516|acc:AF233516.1 mRNA from Homo sapiens (Homo sapiens PD-1-ligand precursor, mRNA, complete cds) (E=2.8e⁻¹⁶⁰).

A NOV4b polypeptide (SEQ ID NO: 22) is 178 amino acid residues and is presented using the one letter code in Table 4E. Signal P, Psort and/or Hydropathy results predict that NOV4b contains a signal peptide and is likely to be localized outside of the cell with a certainty of 0.4180. In other embodiments, NOV4b is localized to the endoplasmic reticulum (membrane) with a certainty of 0.1000, the endoplasmic reticulum (lumen) with a certainty of 0.1000 or the microbody (peroxisome) with a certainty of 0.1000. The most likely cleavage site for a NOV4b peptide is between amino acids 18 and 19, at: LNA-FT.

TABLE 4E

Encoded NOV4b Protein Sequence

(SEQ ID NO:22)

MRIFAVFIFMTYWHLLNAFTVTVPKDLYVVEYFSNMTIECKFPVEKQLDLAALIVYWEMEDKNIIQFVHGEEDLKVQHSSYRQR
ARLLKDQLSLGNAALQITDVKLQDAGVYRCMISYGGADYKRITVKVNAPYNKINQRILVVDPVTSEHELTCQAEGYPKAEVIWT
SSDHQVLSGD

The NOV4b amino acid sequence have 177 of 177 amino acid residues (100%) identical to, and 177 of 177 amino acid residues (100%) similar to, the 290 amino acid residue ptnr:SPTREMBL-ACC:Q9NZQ7 protein from Homo sapiens (Human) (B7-H1 (PD-1-LIGAND PRECURSOR)) (E=1.8e⁻⁹²).

NOV4b is expressed in at least the following tissues: mammalian tissue, uterus. Expression information was derived from the tissue sources of the sequences that were included in the derivation of the sequence of NOV4b.

NOV4a and NOV4b are very closely homologous as is shown in the amino acid alignment in Table 4F.

[image]

[image]

Homologies to any of the above NOV4 proteins will be shared by the other NOV4 proteins insofar as they are homologous to each other as shown above. Any reference to NOV4 is assumed to refer to both of the NOV4 proteins in general, unless otherwise noted.

NOV4a also has homology to the amino acid sequences shown in the BLASTP data listed in Table 4G.

TABLE 4G
BLAST results for NOV4

| Gene Index/ | | Length | Identity | Positives | |
|---|---|---|---|---|---|
| Identifier | Protein/Organism | (aa) | (%) | (%) | Expect |
| ref\|NP_054862.1\| | B7-H1 protein | 290 | 290/290 | 290/290 | e−168 |
| (NM_014143) | [Homo sapiens] | | (100%) | (100%) | |
| ref\|NP_068693.1\| | programmed cell | 290 | 202/291 | 236/291 | e−114 |
| (NM_021893) | death 1 ligand 1 | | (69%) | (80%) | |
| | [Mus musculus] | | | | |
| dbj\|BAA91966.1\| | unnamed protein | 176 | 176/176 | 176/176 | 1e−97 |
| (AK001894) | product [Homo | | (100%) | (100%) | |
| | sapiens] | | | | |
| ref\|NP_079515.1\| | programmed death | 273 | 90/223 | 123/223 | 5e−34 |
| (NM_025239) | ligand 2 [Homo | | (40%) | (54%) | |
| | sapiens] | | | | |
| ref\|NP_067371.1\| | butyrophilin-like | 247 | 90/248 | 132/248 | 1e−28 |
| (NM_021396) | protein [Mus | | (36%) | (52%) | |
| | musculus] | | | | |

The homology of these sequences is shown graphically in the ClustalW analysis shown in Table 4H.

[image]

[image]

Tables 4I and 4J list the domain description from DOMAIN analysis results against NOV4. This indicates that the NOV4 sequence has properties similar to those of other proteins known to contain these domains.

TABLE 4I

Domain Analysis of NOV4

gnl|Smart|smart00409, IG, Immunoglobulin
(SEQ ID NO:83)
Length = 86 residues, 98.8% aligned
Score = 40.8 bits (94), Expect = 1e−04

| | | | |
|---|---|---|---|
| Query: | 24 | PKDLYVVEYGSNMTIECKFPVEKQLDLAALIVYWEMEDKNIIQFVHGEENLKVQHSSYRQ | 83 |
| | | \| + \| \| \| ++\|+ \|+ \| \| \| + + \| \| | |
| Sbjct: | 1 | PPSVTVKE-GESVTLSCEASGNPPPT-----VTWY---KQGGKLLAESGRFSVSRSG--- | 48 |
| Query: | 84 | RARLLKDQLSLGNAALQITDVKLQDAGVYRCMISYGGASYK-RITVKV 130 | |
| | | \|\|+ \| \|++\| +\|+\| \| \| + \|+ \| | |
| Sbjct: | 49 | -----------GNSTLTISNVTPEDSGTYTCAATNSSGSASSGTTLTV 85 | |

TABLE 4J

Domain Analysis of NOV4

gnl|Smart|smart00406, IGv, Immunoglobulin V-Type (SEQ ID NO:84)
Length = 80 residues, 97.5% aligned
Score = 35.4 bits (94), Expect = 0.005

| | | | |
|---|---|---|---|
| Query: | 35 | NMTIECKFPVEKQLDLAALIVYW--EMEDKNIIQFVEGEEDLKVQESSYRQPARLLKDQL | 92 |
| | | ++\|+ \|\| ++ \| \| + \| + + \|+ +\|\|+ \| + \|\| | |
| Sbjct: | 1 | SVTLSCKA---SGFTFSSYYVSWVRQPPGKGLEWLGYIGSDVSYSEASYKGRVTISKD-N | 56 |
| Query: | 93 | SLGNAALQITDVKLQDAGVYRC 114 | |
| | | \| + +\| \|++++++\| \| \| \| | |
| Sbjct: | 57 | SKNDVSLTISNLRVEDTGTYYC 78 | |

Engagement of CTLA4 by B7-1 or B7-2, on the other hand, may inhibit proliferation and interleukin-2 (IL2) production. Antibody against the CD28-related molecule ICOS can stimulate T-cell growth and induce IL10 and IL4 production. By searching an EST database for B7-1 and B7-2 homologs, followed by RT-PCR of a placenta cDNA library, Dong et al. (1999) obtained a cDNA encoding B7H1 (B7 homolog-1). Sequence analysis predicted that the 290-amino acid type I transmembrane protein, which is 20% and 15% identical to B7-1 and B7-2, respectively, has immunoglobulin V-like and C-like domains and a 30-amino acid cytoplasmic tail. Northern blot analysis detected 4.1- and 7.2-kb B7H1 transcripts most abundantly in heart, skeletal muscle, placenta, and lung, with weak expression in thymus, spleen, kidney, and liver, and no expression in brain, colon, and small intestine. Fluorescence-activated cell sorting (FACS) analysis demonstrated B7H1 expression on a fraction of monocytes and, weakly, on T and B cells. Activation significantly increased expression on both T cells and monocytes, and, to a lesser extent, on B cells. Binding analysis demonstrated no interaction between B7H1 and ICOS, CTLA4, or CD28. Stimulation of T cells in the presence of B7H1 enhanced proliferation and the preferential production of IL10 and gamma-interferon (IFNG), but not IL4, in an IL2-dependent manner.

Freeman et al. (2000) also cloned B7H1, which they termed ‘programmed cell death-1 (PDCD1, or PD1) ligand-1,’ or PDL1. Mouse Pd11 is 70% identical to the human protein. Flow cytometric and BIAcore analyses determined that PDL1 binds to PDCD1 , but not to the structurally similar CTLA4, CD28, or ICOS proteins. RNA blot hybridization indicated that PDL1 was upregulated in monocytes by treatment with IFNG and in dendritic cells and keratinocytes by treatment with IFNG together with other activators. In dendritic cells, B7-1 and B7-2 were upregulated in parallel with PDL1. Expression of PDL1 was also upregulated in B cells activated by surface Ig cross-linking. Activation of human T cells and murine Pdcd1 +/+ T cells in the presence of PDL1 led to a decrease in proliferation and cytokine secretion, possibly due to the presence of a cytoplasmic immunoreceptor tyrosine-based inhibitory motif (ITIM) on PDCD1.

By PCR and somatic cell hybrid analysis, Freeman et al. (2000) mapped the PDL1 gene to chromosome 9. Scott (2000) mapped the B7H1 gene to chromosome 9 based on sequence similarity between the B7H1 sequence (GenBank GENBANK AF177937) and the chromosome 9 clone RP11-574F11 (GenBank GENBANK AL162253).

Rennert et al. (1997) Int Immunol 9, 805-13: B7-1 (CD80) and B7-2 (CD86) are genetically and structurally related molecules expressed on antigen-presenting cells. Both bind CD28 to co-stimulate T lymphocytes, resulting in proliferation and cytokine production. The extracellular portions of B7-1 and B7-2 which bind to CD28 and CTLA-4 are related to Ig variable (V) and Ig constant (C) domain sequences. Recent reports have described Splice Variant forms of B7 proteins which occur in vivo and are of unknown function. Here we describe soluble recombinant forms of B7-1 and B7-2 containing either both of the Ig-like extracellular domains or the individual IgV or IgC domains coupled to an Ig Fc tail. Soluble B7-1 and B7-2 bind to CD28 and CTLA-4, and effectively co-stimulate T lymphocytes resulting in their proliferation and the secretion of cytokines. Furthermore, the IgV domain of B7-2 binds CD28 and CTLA-4, competes with B7-1 and B7-2 for binding to these receptors, and co-stimulates T lymphocytes. Cross-linked soluble B7-2v was the most potent co-stimulatory molecule tested and was active at a concentration approximately 100-fold lower than cross-linked soluble B7-1 or B7-2 proteins. When bound to tosyl-activated beads, B7-2v was capable of sustaining multiple rounds of T cell expansion. These data complement the description of naturally occurring variants to suggest that T cell co-stimulation in vivo may be regulated by soluble or truncated forms of B7 proteins.

Several recent studies demonstrate the importance of the co-stimulatory interaction of B7 family members like B7RP-1 (B7 Related Protein-i), B7-1, and B7-2, with antigen receptors such as CD28, CTLA-4 (Cytotoxic T Lymphocyte-associated Antigen 4) and ICOS (Inducible Co-Stimulatory molecule). These protein interactions have been shown to be critical for normal T-cell activation and proliferation, B-cell stimulation and antibody production, immunoglobulin class switching, interleukin production, and germinal center formation. Because these events constitute critical steps in mediating proper humoral immune responses, their modulation may serve as potent therapeutics for immune system disorders of many kinds (Dong, C., et al. ICOS co-stimulatory receptor is essential for T-cell activation and function. Nature. 409, 97-101 (2001).; McAdam, A. J., et al. ICOS is critical for CD40-mediated antibody class switching. Nature. 409, 102-105 (2001).; Tafuri, A., et al. ICOS is essential for effective T-helper-cell responses. Nature. 409, 105-109 (2001).; Yoshinaga, S. K., et al. T-cell co-stimulation through B7RP-1 and ICOS. Nature. 402, 827-932 (1999).)

The B7 family members B7-1 and B7-2 interact with CD28 and constitute an essential T-cell co-stimulatory pathway in the initiation of antigen-specific humoral and cell-mediated immune response. Here, we describe a third member of the B7 family, called B7-H1 that does not bind CD28, cytotoxic T-lymphocyte A4 or ICOS (inducible co-stimulator). Ligation of B7-H1 co-stimulated T-cell responses to polyclonal stimuli and allogenic antigens, and preferentially stimulated the production of interleukin-10. Interleukin-2, although produced in small amounts, was required for the effect of B7-H1 co-stimulation. Our studies thus define a previously unknown co-stimulatory molecule that may be involved in the negative regulation of cell-mediated immune responses. PMID: 10581077, UI: 20048154

Costimulation is critical to T cell activation. On the antigen-presenting cell the key players are found in the extended family of B7 genes comprising cd80, cd86, B7h/B7RP-1 and B7-H1. cd80 and cd86 encode proteins that bind to CD28 and CTLA4 on T cells. Blocking this pathway with the potent CTLA4-Ig fusion protein shows encouraging potential as a therapeutic agent. While cd80 and cd86 pathways act mainly on naive T cells, B7h/B7RP-1 and B7-H1 seem to exert their effects on antigen-experienced lymphocytes. PMID: 11029388, UI: 20485717

Engagement of CD28 (OMIM #186760) by B7-1 (CD80; OMIM #112203) or B7-2 (CD86; OMIM #601020) in the presence of antigen promotes T-cell proliferation, cytokine production, differentiation of effector T cells, and the induction of BCL-X (OMIM #600039), a promoter of T-cell survival. Engagement of CTLA4 (OMIM #123890) by B7-1 or B7-2, on the other hand, may inhibit proliferation and interleukin-2 (IL2; OMIM #147680) production. Antibody against the CD28-related molecule ICOS (OMIM #604558) can stimulate T-cell growth and induce IL10 (OMIM #124092) and IL4 (OMIM #147780) production. By searching an EST database for B7-1 and B7-2 homologs, followed by RT-PCR of a placenta cDNA library, Dong et al. (1999) obtained a cDNA encoding B7-H1 (B7 homolog-1). Sequence analysis predicted that the 290-amino acid type I transmembrane protein, which is 20% and 15% identical to B7-1 and B7-2, respectively, has immunoglobulin V-like and C-like domains and a 30-amino acid cytoplasmic tail. Northern blot analysis detected 4.1- and 7.2-kb B7-H1 transcripts most abundantly in heart, skeletal muscle, placenta, and lung, with weak expression in thymus, spleen, kidney, and liver, and no expression in brain, colon, and small intestine. Fluorescence-activated cell sorting (FACS) analysis demonstrated B7-H 1 expression on a fraction of monocytes and, weakly, on T and B cells. Activation significantly increased expression on both T cells and monocytes, and, to a lesser extent, on B cells. Binding analysis demonstrated no interaction between B7-H1 and ICOS, CTLA4, or CD28. Stimulation of T cells in the presence of B7-H1 enhanced proliferation and the preferential production of IL10 and gamma-interferon (IFNG; OMIM #147570), but not IL4, in an IL2-dependent manner.

The protein similarity information, expression pattern, and map location for the NOV4 protein and nucleic acid disclosed herein suggest that it may have important structural and/or physiological functions characteristic of the B7 family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: brain disorders including epilepsy, eating disorders, schizophrenia, ADD, and cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, and cancer; pancreatic disorders including pancreatic insufficiency and cancer; and prostate disorders including prostate cancer, immune-mediated pathogenesis, T-cell-mediated diseases, multiple sclerosis, colitis, cancer, trauma, regeneration (in vitro and in vivo), viral/bacterial/parasitic infections and other diseases, disorders and conditions of the like.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV4 protein has multiple hydrophilic regions, each of which can be used as an immunogen. In one embodiment, a contemplated NOV4 epitope is from about amino acids 40 to 45. In another embodiment, a contemplated NOV4 epitope is from about amino acids 52 to 55. In other specific embodiments, contemplated NOV4 epitopes are from about amino acids 60 to 68, 70 to 90, 110 to 112, 130 to 140, 142 to 145, 150 to 155, 157 to 160, 175 to 190, 220 to 240 and 260 to 280.

NOV5

NOV5 includes two novel prostasin-like proteins. The disclosed proteins have been named NOV5a and NOV5b.

NOV5a

A disclosed NOV5a nucleic acid (designated as CuraGen Acc. No. CG56142-01), encodes a novel prostasin-like protein and includes the 866 nucleotide sequence (SEQ ID NO: 23) shown in Table 5A. An open reading frame for the mature protein was identified beginning with an ATG codon at nucleotides 19-21 and ending with a TGA codon at nucleotides 820-822. Putative untranslated regions downstream from the termination codon and upstream from the initiation codon are underlined in Table 2A, and the start and stop codons are in bold letters.

TABLE 5A

NOV5a Nucleotide Sequence

(SEQ ID NO:23)

CCCAGCCTTGAAGACAGAATGAGAGGGGTTTCCTGTCTCCAGGTCCTGCTCCTTCTGGTGCTGGCCTGCGGGCAGCCCCGCATG
TCCAGTCGGATCGTTGGGGGCCGGGATGGCCGGGACGGAGAGTGGCCGTGGCAGGCGAGCATCCAGCATCGTGGGGCACACGTG
TGCGGGGGGTCGCTCATCGCCCCCCAGTGGGTGCTGACAGCGGCGCACTGCTTCCCCAGGGCACTGCCAGCTGAGTACCGCGTG
CGCCTGGGGGCGCTGCGTCTGGGCTCCACCTCGCCCCGCACGCTCTCGGTGCCCGTGCGACGGGTGCTGCTGCCCCCGGACTAC
TCCGAGGACGGGGCCCGCGGCGACCTGGCACTGCTGCAGCTGCGTCGCCCGGTGCCCCTGAGCGCTCGCGTCCAACCCGTCTGC
CTGCCCGTGCCCGGCGCCCGCCCGCCGCCCGGCACACCATGCCGGGTCACCGGCTGGGGCAGCCTCCGCCCAGGAGTGCCCCTC
CCAGAGTGGCGACCGCTACAAGGAGTAAGGGTGCCGCTGCTGGACTCGCGCACCTGCGACGGCCTCTACCACGTGGGCGCGGAC
GTGCCCCAGGCTGAGCGCATTGTGCTGCCTGGGAGTCTGTGTGCCGGCTACCCCCAGGGCCACAAGGACGCCTGCCAGGTGTGC
ACCCAGCCTCCCCAGCCTCCGGAGTCCCCTCCCTGTGCCCAGCACCCTCCCTCCCTGAACTCCAGGACCCAGGACATCCCAACT
CAGGCTCAGGATCCTGGCCTCCAACCTAGAGGCACCACGCCAGGGGTCTGGAACCCTGAGAACTGAAGTCCTGGGAGGGCTGGG
ACTTAGGCTCCTCTTTCTCCTGCAGG

The nucleic acid sequence of NOV5a maps to chromosome 16 has 421 of 639 bases (65%) identical to a gb:GENBANK-ID:AF175522|acc:A-F175522.1 mRNA from Homo sapiens (Homo sapiens transmembrane trytase mRNA, complete cds) (E=1.2e⁻³⁰).

The NOV5a polypeptide (SEQ ID NO: 25) is 267 amino acid residues in length and is presented using the one-letter amino acid code in Table 5B. The SignalP, Psort and/or Hydropathy results predict that NOV5a has a signal peptide and is likely to be localized outside of the cell with a certainty of 0.6902. In alternative embodiments, a NOV5a polypeptide is located to the endoplasmic reticulum (membrane) with a certainty of 0.1000, the endoplasmic reticulum (lumen) with a certainty of 0.1000, or the lysosome (lumen) with a certainty of 0.1000. The SignalP predicts a likely cleavage site for a NOV5a peptide between amino acid positions 18 and 19, i.e. at the dash in the sequence ACG-QP.

TABLE 5B

Encoded NOVSa Protein Sequence

(SEQ ID NO:24)

MRGVSCLQVLLLLVLACGQPRMSSRIVGGRDGRDGEWPWQASIQHRGAHVCGGSLIAPQNVLTAAICFPRALPAEYRVRLG
ALRLGSTSPRTLSVPVRRVLLPPDYSEDGARGDLALLQLRRPVPLSARVQPVCLPVPGARPPPGTPCRVTGWGSLRPGVPL
PEWRPLQGVRVPLLDSRTCDGLYHVGADVPQAERIVLPGSLCAGYPQGHKDACQVCTQPPQPPESPPCAQHPPSLNSRTQD
IPTQAQDPGLQPRGTTPGVWNPEN

The NOV5a amino acid sequence has 93 of 201 amino acid residues (46%) identical to, and 125 of 201 amino acid residues (62%) similar to, the 342 amino acid residue ptnr:TREMBLNEW-ACC:AAG32641 protein from Rattus norvegicus (Rat) (PROSTASIN) (E=1.2e⁻⁴⁵).

NOV5a is expressed in at least the following tissues: endometrium cancer tissue. This information was derived by determining the tissue sources of the sequences that were included in the invention including but not limited to SeqCalling sources, Public EST sources, Literature sources, and/or RACE sources.

Possible small nucleotide polymorphisms (SNPs) found for NOV5a are listed in Tables 5C.

TABLE 5C
SNPs

| | Nucleotide | Base | Amino Acid | Base |
|---|---|---|---|---|
| Variant | Position | Change | Position | Change |
| 13376578 | 736 | A > G | 240 | Arg > Gly |

NOV5b

A disclose NOV5b nucleic acid (designated as CuraGen Acc. No. CG56142-02), encodes a novel prostasin-like protein and includes the 1020 nucleotide sequence (SEQ ID NO: 25) shown in Table 5D. An open reading frame for the mature protein was identified beginning with an ATG codon at nucleotides 91-93 and ending with a TAA codon at nucleotides 931-933. The start and stop codons of the open reading frame are highlighted in bold type. Putative untranslated regions are underlined and found upstream from the initiation codon and downstream from the termination codon.

TABLE 5D
NOVSb Nucleotide Sequence

(SEQ ID NO:25)

AGGACTCTCCTCTCTTCTCCCTGCTGGCTCCAGACCAGAGTCCAAGCCCTAGGCAGTGCCACCCTTACCCAGCCCAGC
CTTGAAGACAGAATGAGACGGGTTTCCTGTCTCCAGGTCCTQCTCCTTCTGGTGCTCCGACCTCCTGGGACTCAGGGA
AGGAAGTCTGCAGCCTGCGGGCAGCCCCGCATGTCCAGTCGGATCGTTGGGGGCCGGGATGGCCGGGACCGAGAGTGG
CCGTGGCAGGCGAGCATCCAGCATCGTGGGGCACACGTGTGCGCGQGGTCGCTCATCGCCCCCCAGTGGGTGCTGACA
GCGGCGCACTGCTTCCCCAGGAGGGCACTGCCAGCTCAGTACCGCGTGCGCCTGGGGGCGCTGCGTCTGGGCTCCACC
TCGCCCCGCACGCTCTCGGTGCCCGTGCGACGGGTGCTGCTGCCCCCGGACTACTCCGAGGACGGGGCCCGCGGCGAC
CTGGCACTGCTGCAGCTGCGTCGCCCGGTGCCCCTGAGCGCTCGCGTCCAACCCGTCTCCCTGCCCGTGCCCGGCGCC
CGCCCGCCGCCCGGCACACCATGCCGGGTCACCGGCTGGGGCAGCCTCCGCCCAGGAGTGCCCCTCCCAGAGTGGCGA
CCGCTACAAGGAGTAAGGGTGCCGCTGCTGGACTCGCGCACCTGCGACGGCCTCTACCACGTGGGCGCGGACGTGCCC
CAGGCTGAGCGCATTGTGCTGCCTGGGAGTCTGTGTCCGGCTACCCCCAGGGCCACAAGAACGCCTGCCAGGGTGAT
TCTGGGGGACCTCTGACCTGCCTGCAGTCTGGGAGCTGGGTCCTGGTGGGCGTGGTGAQCTGGGGCAAGGGTTGTGCC
CTGCCCAACCGTCCAGGGGTCTACACCAGTGTGGCCACATATAGCCCCTGGATTCAGGCTCGCGTCAGCTTCTAATGC
TAGCCGGTGAGGCTGACCTGGAGCCAGCTGCTGGGGTCCCTCAGCCTCCTGGTTCATCCAGGCACCTGCCTATACCCC
ACATCC

The nucleic acid sequence of NOV5bmaps to chromosome 16 has 561 of 863 bases (65%) identical to a gb:GENBANK-ID:HSA306593|acc:AJ306593.1 mRNA from Homo sapiens (Homo sapiens mRNA for marapsin (MPN gene)) (E=4.8e⁻⁴⁷).

The NOV5b polypeptide (SEQ ID NO: 26) is 280 amino acid residues in length and is presented using the one-letter amino acid code in Table 5E. The SignalP, Psort and/or Hydropathy results predict that NOV5b has a signal peptide and is likely to be localized endoplasmic reticulum (membrane) with a certainty of 0.8200. In alternative embodiments, a NOV5b polypeptide is located to the plasma membrane with a certainty of 0.1900, the endoplasmic reticulum (lumen) with a certainty of 0.1000, or the outside of the cell with a certainty of 0.1000. The SignalP predicts a likely cleavage site for a NOV5b peptide between amino acid positions 22 and 23, i.e. at the dash in the sequence TQG-RK.

TABLE 5E
Encoded NOV5b Protein Sequence

(SEQ ID NO:26)

MRGVSCLQVLLLLVLGAAGTQGRKSAACGQPRMSSRIVGGRDGRDGEWPWQASIQHRGAHVCGGSLIAPQWVLTAAHCRPRRAL
PAEYRVRLGALRLGSTSPRTLSVPVRRVLLPPDYSEDGARGDLALLQLRRPVPLSARVQPVCLPVPGARPPPGTPCRVTGWGSL
RPGVPLPEWRPLQGVRVPLLDSRTCDGLYHVGADVPQAERIVLPGSLCAGYPQGHKDACQGDSGGPLTCLQSHSWVLVGVVSWG
KGCALPNRPGVYTSVATYSPWIQARVSF

The NOV5b amino acid sequence to has 132 of 276 amino acid residues (47%) identical to, and 172 of 276 amino acid residues (62%) similar to, the 342 amino acid residue ptnr:SPTREMBL-ACC:Q9ES87 protein from Rattus norvegicus (Rat) (PROSTASIN) (E=6.9e⁻⁶⁷).

NOV5a and NOV5b are very closely homologous as is shown in the amino acid alignment in Table 5F.

[image]

Homologies to any of the above NOV5 proteins will be shared by the other NOV5 proteins insofar as they are homologous to each other as shown above. Any reference to NOV5 is assumed to refer to both of the NOV5 proteins in general, unless otherwise noted.

NOV5a also has homology to the amino acid sequences shown in the BLASTP data listed in Table 5G.

TABLE 5G
BLAST results for NOV5

| Gene Index/Identifier | Protein/Organism | Length (aa) | Identity (%) | Positives (%) | Expect |
|---|---|---|---|---|---|
| dbj\|BAB08216.1\| | embryonic serine | 317 | 76/201 | 112/201 | 4e−36 |
| (AB038496) | protease-1 | | (37%) | (54%) | |
| | [Xenopus laevis] | | | | |
| gb\|AAG32641.1\|AF2 | prostasin | 342 | 85/202 | 116/202 | 3e−35 |
| 02076_1 | [Rattus | | (42%) | (57%) | |
| (AF202076) | norvegicus] | | | | |
| sp\|Q9ES87\| | Prostasin | 342 | 85/202 | 116/202 | 3e−35 |
| PSS8_RAT | precursor | | (42%) | (57%) | |
| ref\|NP_114154.1\| | marapsin [Homo | 290 | 85/202 | 113/202 | 5e−35 |
| (NM_031948) | sapiens] | | (42%) | (55%) | |
| gb\|AAH03851.1\|AAH | Similar to | 339 | 85/202 | 117/202 | 1e−34 |
| 03851 (BC003851) | protease, | | (42%) | (57%) | |
| | serine, 8 | | | | |
| | (prostasin) [Mus | | | | |
| | musculus] | | | | |

The homology of these sequences is shown graphically in the ClustalW analysis shown in Table 5H.

[image]

[image]

The protein similarity information, expression pattern, and map location for the NOV5 protein and nucleic acid disclosed herein suggest that it may have important structural and/or physiological functions characteristic of the family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, cancer, trauma, regeneration (in vitro and in vivo), viral/bacterial/parasitic infections, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, aneurysm, fibromuscular dysplasia, stroke, anemia, bleeding disorders, adrenoleukodystrophy, congenital adrenal hyperplasia, diabetes, Von Hippel-Lindau (VHL) syndrome, pancreatitis, hyperparathyroidism, hypoparathyroidism, SIDS, endometriosis, fertility, xerostomia, hypercalceimia, ulcers, cirrhosis, inflammatory bowel disease, diverticular disease, Hirschsprung's disease, Crohn's Disease, appendicitis, hemophilia, hypercoagulation, idiopathic thrombocytopenic purpura, autoimmume disease, allergies, immunodeficiencies, graft vesus host, ataxia-telangiectasia, hemophilia, lymphedema, tonsilitis, osteoporosis, arthritis, ankylosing spondylitis, scoliosis, tendinitis, muscular dystrophy, Lesch-Nyhan syndrome, Myasthenia gravis, dental disease and infection, Alzheimer's disease, tuberous sclerosis, Parkinson's disease, Huntington's disease, cerebral palsy, epilepsy, Lesch-Nyhan syndrome, multiple sclerosis, Ataxia-telangiectasia, leukodystrophies, behavioral disorders, addiction, anxiety, pain, neuroprotection, growth and reproductive disorders, endocrine dysfunctions, systemic lupus erythematosus, asthma, emphysema, ARDS, pharyngitis, laryngitis, hearing loss, tinnitus, psoriasis, actinic keratosis, tuberous sclerosis, acne, hair growth, allopecia, pigmentation disorders, cystitis, incontinence, renal artery stenosis, interstitial nephritis, glomerulonephritis, polycystic kidney disease, systemic lupus erythematosus, renal tubular acidosis, IgA nephropathy, vesicoureteral reflux, glaucoma, blindness, and hypothyroidism ventricular septal defect (VSD), valve diseases, tuberous sclerosis, scleroderma, obesity, transplantation and other diseases, disorders and conditions of the like.

The novel nucleic acid encoding the prostasin-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV5 protein has multiple hydrophilic regions, each of which can be used as an immunogen. In one embodiment, a contemplated NOV5 epitope is from about amino acids 30 to 35. In another embodiment, a contemplated NOV5 epitope is from about amino acids 40 to 45. In other specific embodiments, contemplated NOV5 epitopes are from about amino acids 70 to 80, 95 to 105, 110 to 115, 140 to 150, 160 to 170, 175 to 180, 190 to 195, 220 to 225, 230 to 240, 245 to 248, 249to 252 and 260 to 262.

NOV6

NOV6 includes three novel lysosomal acid lipase-like proteins. The disclosed proteins have been named NOV6a and NOV6b.

NOV6a

A disclosed NOV6a nucleic acid (designated as CuraGen Acc. No. CG50159-01), encodes a novel lysosomal acid lipase-like protein and includes the 1267 nucleotide sequence (SEQ ID NO: 27) shown in Table 6A. An open reading frame for the mature protein was identified beginning with an ATG codon at nucleotides 9-10 and ending with a TAA codon at nucleotides 1127-1129. Putative untranslated regions downstream from the termination codon and upstream from the initiation codon are underlined in Table 6A, and the start and stop codons are in bold letters.

TABLE 6A
NOV6a Nucleotide Sequence

(SEQ ID NO:27)

GTCCAAAATGTGGCTGCTTTTAACAACAACTTGTTTGATCTGTGGAACTTTAAATGCTGGTGGATTCCTTGATTTGGAAAAGTGA
AGTGAATCCTGAGGTGTGGATGAATACTAGTGAAATCATCATCTACAATGGCTACCCCAGTGAAGAGTATGAAGTCACCACTGA
AGATGGGTATATACTCCTTGTCAACAGAATTCCTTATGGGCGAACACATGCTAGGAGCACAGGTCCCCGGCCAGTTGTGTATAT
GCAGCATGCCCTGTTTGCAGACAATGCCTACTGGCTTGAGAATTATGCTAATGGAAGCCTTGGATTCCTTCTAGCAGATGCAGG
TTATGATGTATGGATGGGAAACAGTCGGGGAAACACTTGGTCAAGAAGACACAAAACACTCTCAGAGACAGATGAGAAATTCTG
GGCCTTTGGTTTTGATGAAATGGCCAAATATGATCTCCCAGGAGTAATAGACTTCATTGTAAATAAAACTGGTCAGGAGAAATT
GTATTTCATTGGACATTCACTTGGCACTACAATAGGGTTTGTAGCCTTTTCCACCATGCCTGAACTGGCACAAAGAATCAAAAT
GAATTTTGCCTTGGGTCCTACGATCTCATTCAAATATCCCACGGGCATTTTTACCAGGTTTTTTCTACTTCCAAATTCCATAAT
CAAGGCTGTTTTTTGGTACCAAAGGTTTCTTTTTAGAAGATAAGAAAACGAAGATAGCTTCTACCAAAATCTGCAACAATAAGAT
ACTCTGGTTGATATGTAGCGAATTTATGTCCTTATGGGCTGGATCCAACAAGAAAAATATGAATCAGCTTTACCACTCTTGATGA
ATTCAGAGCTTATGACTGGGGAAATGACGCTGATAATATGAAACATTACAATCAGAGTCTCCCCCTATATATGACCTGACTGC
CATGAAAGTGCCTACTGTATTTGGGCTGGTGGACATGAGTCCTCGTAACACCCCAGGATGTGGCCAGGATACTCCCTCAAAT
CAAGAGTCTTCATTACTTTAAGCTATTGCCAGATTGGAACCACTTTGATTTGTCTGGGGCCTCGATGCCCCTCAACGATGTA
CAGTGAAATCATAGCTTTAATGAAGGCATATTCCTAAATGCAATGCATTTACTTTTCAATTAAAAGTTGCTTCCAAGCCATAA
GGGACTTTAGAAAAAATAGTAACCAACAATGAGGTTGTCCCCAGCACCCTGGGGGAGATGCACAGTGGAGTCTGTTTTCCAAG
TCAATG

The nucleic acid sequence of NOV6a maps to chromosome 10 and has 545 of 820 bases (66%) identical to a gb:GENBANK-ID:RNLIP|acc:X02309.1 mRNA from Rattus norvegicus (Rat mRNA for lingual lipase) (E=2.5e⁻⁷¹).

The NOV6a polypeptide (SEQ ID NO: 28) is 373 amino acid residues in length and is presented using the one-letter amino acid code in Table 6B. The SignalP, Psort and/or Hydropathy results predict that NOV6a has a signal peptide and is likely to be localized to the lysome (lumen) with a certainty of 0.5500. In alternative embodiments, a NOV6a polypeptide is located to the outside of the cell with a certainty of 0.3700, the microbody (peroxisome) with a certainty of 0.2967, or the endoplasmic reticulum (membrane) with a certainty of 0.1000. The SignalP predicts a likely cleavage site for a NOV6a peptide between amino acid positions 17 and 18, i.e. at the dash in the sequence LNA-GG.

TABLE 6B
Encoded NOV6a Protein Sequence

(SEQ ID NO:28)

MWLLLTTTCLICGTLNAGGFLDLENEVNPEVWMNTSEIIIYNGYPSEEYEVTTEDGYILLVNRIPYGRTHARSTGPRPVVY
MQHALFADNAYWLENYANGSLGFFLLADAGYDVWMGNSRGNTWWSRRHKTLSETDEKFWAFGFDEMAKYDLPGVIDFIVNKTG
QEKLYFIGHSLGTTIGFAVAFSTMPELAQRIKMNFALGPTISFKYPTGIFTRFFLLPNSIIKAVFGTKGFFLEDKKTKIAST
KICNNKILWLICSEFMSLWAGSNKKNMNQLYHSDEFRAYDWGNDADNMKHYNQSHPIYDLTAMKVPTAIWAGGHDVLVTP
QDVARILPQIKSLHYFKLLPDWNHFDFVWGLDAPQRMYSEIIALMKAYS

The NOV6a amino acid sequence has 152 of 297 amino acid residues (51%) identical to, and 201 of 297 amino acid residues (67%) similar to, the 399 amino acid residue ptnr:SPTREMBL-ACC:Q16529 protein from Homo sapiens (Human) (LYSOSOMAL ACID LIPASE PRESCURSOR) (E=6.2e⁻¹⁰⁸).

Possible small nucleotide polymorphisms (SNPs) found for NOV6 are listed in Table 6C.

TABLE 6C
SNPs

| | Nucleotide | Base | Amino Acid | |
|---|---|---|---|---|
| Variant | Position | Change | Position | Base Change |
| 13375591 | 191 | A > G | 62 | Asn > Asp |
| 13375592 | 221 | A > G | 72 | Arg > Gly |
| 13373919 | 299 | G > C | 998 | Ala > Pro |
| 13373884 | 301 | T > C | NA | NA |
| 13373921 | 399 | C > T | 131 | Ser > Leu |
| 13375593 | 428 | G > A | 141 | Gly > Ser |
| 13375594 | 735 | C > A | 243 | Thr > Asn |
| 13375595 | 867 | A > G | 287 | Asp > Gly |

NOV6b

A disclosed NOV6b nucleic acid (designated as CuraGen Acc. No. CG50159-02), encodes a novel lysosomal acid lipase-like protein and includes the 1267 nucleotide sequence (SEQ ID NO: 29) shown in Table 6D. An open reading frame for the mature protein was identified beginning with an ATG codon at nucleotides 8-10 and ending with a TAA codon at nucleotides 1126-1128. The start and stop codons of the open reading frame are highlighted in bold type. Putative untranslated regions are underlined and found upstream from the initiation codon and downstream from the termination codon.

TABLE 6D
NOV6b Nucleotide Sequence

(SEQ ID NO:29)

GTCCAAAATGTGGCTGCTTTTAACAACAACTTGTTTGATCTGTGGAACTTTAAATGCTGGTGGATTCCTTGATTTCGA
AAATGAAGTGAATCCTGAGGTGTGGATGAATACTAGTGAAATCATCATCTACAATGGCTACCCCAGTGAAGAGTATGA
AGTCACCACTGAAGATGGGTATATACTCCTTGTCGACAGAATTCCTTATGGGCGAACACATGCTGGGAGCACAGGTCC
CCGGCCAGTTGTGTATATGCAGCATGCCCTGTTTGCAGACAATGCCTACTGGCTTGAGAATTATCCTAATGGAAGCCT
TGGATTCCTTCTAGCAGATGCAGGTTATGATGTATGGATGGGAAACAGTCGGGGAAACACTTGGTCAAGAAGACACAA
AACACTCTCAGAGACAGATGAGAAATTTCTGGGCCTTTAGTTTTGATGAAATGGCCAAATATGATCTCCCAGGAGTAAT
AGACTTCATTGTAAATAAAACTGGTCAGGAGAAATTGTATTTCATTGGACATTCACTTGGCACTACAATAGGGTTTGT
AGCCTTTTCCACCATGCCTGAACTGGCACAAAGAATCAAAATGAATTTTGCCTTGGGTCCTACGATCTCATTCAAATA
TCCCACGGGCATTTTTACCAGGTTTTTTCTACTTCCAAATTCCATAATCAAGGCTGTTTTTGGTACCAAAGGTTTCTT
TTTAGAAGATAAGAAAACGAAGATAGCTTCTAACAAAATCTGCAACAATAAGATACTCTGGTTGATATGTAGCGAATT
TATGTCCTTATGGGCTGGATCCAACAAGAAAAATATGAATCAGCTTTACCACTCTGATGAATTCAGAGCTTATGACTG
GGGAAATGGCGCTGATAATATGAACATTACAATCAGAGTCATCCCCCCTATATATGACCTGACTGCCATGAAAGTGCC
TACTGCTATTTGGGCTGGTGGACATGATGTCCTCGTAACACCCCAGGATGTGGCCAGGATACTCCCTCAAATCAAGAG
TCTTCATTACTTTAAGCTATTGCCAGATTGGAACCACTTTGATTTTGTCTGGGGCCTCGATGCCCCTCAACGGATGTA
CAGTGAAATCATAGCTTTAATGAAGGCATATTCCTAAATGCAATGCATTTACTTTTCAATTAAAAGTTGCTTCCAAGC
CCATAAGGGACTTTAGAAAAAATAGTAACCAACAATGAGGTTGTCCCCCAGCACCCTGGGGGAGATGCACAGTGGAGT
CTGTTTTCCAAGTCAATTG

The nucleic acid sequence of NOV6b maps to chromosome 17 and has 545 of 820 bases (66%) identical to a gb:GENBANK-ID:RNLIP|acc:X02309.1 mRNA from Rattus norvegicus (Rat mRNA for lingual lipase) (E=2.5e⁻⁷¹).

The NOV6b polypeptide (SEQ ID NO: 30) is 373 amino acid residues in length and is presented using the one-letter amino acid code in Table 6E. The SignalP, Psort and/or Hydropathy results predict that NOV6b has a signal peptide and is likely to be localized to the lysosome (lumen) with a certainty of 0.5500. In alternative embodiments, a NOV6b polypeptide is located to the outside of the cell with a certainty of 0.3700, the microbody (peroxisome) with a certainty of 0.2967, or the endoplasmic reticulum (membrane) with a certainty of 0.1000. The SignalP predicts a likely cleavage site for a NOV6b peptide between amino acid positions 17 and 18, i.e. at the dash in the sequence LNA-GG.

TABLE 6E
Encoded NOV6b Protein Sequence

(SEQ ID NO:30)

MWLLLTTTCLICGTLNAGGFLDLENEVNPEVWMNTSEIIIYNGYPSEEYEVTTEDGYILLVDRIPYGRTHAGSTGPRPVVYMQH
ALFADNAYWLENYPNGSLGFLLADAGYDVWNGNSRGNTWSRRHKTLSETDEKFWAFSFDEAMAKYDLPGVIDFIVNKTGQEKLYF
IGHSLGTTIGFVAFSTMPELAQRIKMNFALGPTISFKYPTGIFTRFFLLPNSIIKAVFGTKGFFLEDKKTKIASNKICNNKILW
LICSEFMSLWAGSNKKWMNQLYHSDEFRAYDWGNADNMKHYNQSHPPIYDLTAMKVPTAIWAGGHDVLVTPQDVARILPQIKS
LHYFKLLPDWNEFDFVWGLDAPQRNYSEIIALMKAYS

The NOV6b amino acid sequence has152 of 297 amino acid residues (51%) identical to, and 201 of 297 amino acid residues (67%) similar to, the 399 amino acid residue ptnr:SPTREMBL-ACC:Q16529 protein from Homo sapiens (Human) (LYSOSOMAL ACID LIPASE PRECURSOR) (E=6.2e⁻¹⁰⁸).

NOV6c

A disclosed NOV6c nucleic acid (designated as CuraGen Acc. No. CG50159-04), encodes a novel lysosomal acid lipase-like protein and includes the 1195 nucleotide sequence (SEQ ID NO: 30) shown in Table 6F. An open reading frame for the mature protein was identified beginning with an ATG codon at nucleotides 8-10 and ending with a TAA codon at nucleotides 1126-1128. The start and stop codons of the open reading frame are highlighted in bold type. Putative untranslated regions are underlined and found upstream from the initiation codon and downstream from the termination codon.

TABLE 6F
NOV6c Nucleotide Sequence

(SEQ ID NO:31)

GTCCAAAATGTGGCTGCTTTTAACAACAACTTGTTTGATCTGTGGAACTTTAAATGCTGGTGGATTCCTTGATTTGGA
AAATGAAGTGAATCCTGAGGTGTGGATGAATACTAGTGAAATCATCATCTACAATGGCTACCCCAGTGAAGAGTATGA
AGTCACCACTGAAGATGGGTATATCTCCTTGTCAACAGAATTCCTTATGGGCGAACACATGCTAGGAGCACAGGTTCC
CCGGCCAGTTGTGTATATGCAGCATGCCTGTTTGCASGACAATGCCTACTGGCTTGAGAATTATGCTAATGGAAGCCT
TGGATTCCTTCTAGCAGATGCAGGTTATGATGTATGGATGGGAAACAGTCGGGGAAACACTTGGTCAAGAAGACACAA
AACACTCTCAGAGACAGATGAGAAATTCTGGGCCTTTGGTTTTGATGAAATGGCCAAATATGATCTCCCAGGAGTAAT
AGACTTCATTGTAAATAAAACTGGTCAGGAGAAATTGTATTTCATTGGACATTCACTTGGCACTACAATAGGGTTTGT
AGCCTTTTCCACCATGCCTGAACTGGCACAAAGAATCAAAATGAATTTTGCCTTGGGTCCTACGATCTCATTCAAATA
TCCCACGGGCATTTTTACCAGGTTTTTTCTACTTCCAAATTCCATAATCAAGGCTGTTTTTGGTACCAAAGGTTTCTT
TTTAGAAGATAAGAAAACGAAGATAGCTTCTACCAAAATCTGCAACAATAAGATACTCTGGTTGATATGTAGCGAATT
TATGTCCTTATGGGCTGGATCCAACAAGAAAAATATGAATCAGAGTCATCCCCCTATATATGACCTGACTGCCATGGA
AGTGCCTACTGCTATTTGGGCTGGTGGACATGATGTCCTCGTAACACCCCAGGATGTGGCCAGGATACTCCCTCAAAT
CAAGAGTCTTCATTACTTTAAGCTATTGCCAGATTGGAACCACTTTGATTTTGTCTGGGGCCTCGATGCCCCTCAACG
GATGTACAGTGAAATCATAGCTTTAATGAAGGCATATTCCTAAATGCAATGCATTTACTTTTCGATTAAAAGTTGCTT
CCAAGCCCATAAGGGACTTTAGAAAAAATAGTAACCAACAATGAGGTTGTCCCCCAGCAACCTGGGGGAGATGCACAG
TGGAGTCTGTTTTCCAAGTCAATTG

The nucleic acid sequence of NOV6c maps to chromosome 10 and has 557 of 827 bases (67%) identical to a gb:GENBANK-ID:A01046|acc:A01046.1 mRNA from Homo sapiens (H.sapiens mRNA for human gastric lipase) (E=2.5e⁻⁷¹).

The NOV6c polypeptide (SEQ ID NO: 30) is 349 amino acid residues in length and is presented using the one-letter amino acid code in Table 6G. The SignalP, Psort and/or Hydropathy results predict that NOV6c has a signal peptide and is likely to be localized to the lysosome (lumen) with a certainty of 0.8306. In alternative embodiments, a NOV6c polypeptide is located to the outside of the cell with a certainty of 0.3700, the microbody (peroxisome) with a certainty of 0.2944, or the endoplasmic reticulum (membrane) with a certainty of 0.1000. The SignalP predicts a likely cleavage site for a NOV6b peptide between amino acid positions 17 and 18, i.e. at the dash in the sequence LNA-GG.

TABLE 6G
Encoded NOV6c Protein Sequence

(SEQ ID NO:32)

MWLLLTTTCLICGTLNAGGFLDLENEVNPEVWMNTSEIIIYNGYPSEEYEVTTEDGYILLVNRIPYGRTHARSTGPRPVVYMQH
ALFADNAYWLENYANGSLGFLLADAGYDVWMGNSRGNTWSRRHKTLSETDEKFWAFGFDEMAKYDLPGVIDFIVNKTGQEKLYF
IGHSLGTTIGFVAFSTMPELAQRIKMNFALGPTISFKYPTGIFTRFFLLPNSIIKAVGFGTKGFFLEDKKTKIASTKICNNKILW
LICSEFMSLWAGSNKKNMNQSHPPIYDLTAMKVPTAIWAGGHDVLVTPQDVARILPQIKSLHYFKLLPDWNHFDFVWGLDAPQR
MYSEIIALMKAYS

The NOV6c amino acid sequence has143 of 278 amino acid residues (51%) identical to, and 185 of 278 acid residues (66%) similar to, the 395 amino acid residue ptnr:SPTREMBL-ACC:Q9D798 protein from Mus musculus (Mouse) (2310051B21RIK PROTEIN) (E=7.2e⁻⁹⁹).

NOV6c is expressed in at least the following tissues: pooled mammalian tissues. Expression information was derived from the tissue sources of the sequences that were included in the derivation of the NOV6c sequence.

NOV6a, NOV6b and NOV6c are very closely homologous as is shown in the amino acid alignment in Table 6H.

[image]

[image]

Homologies to any of the above NOV6 proteins will be shared by the other NOV6 proteins insofar as they are homologous to each other as shown above. Any reference to NOV6 is assumed to refer to both of the NOV6 proteins in general, unless otherwise noted.

NOV6a also has homology to the amino acid sequences shown in the BLASTP data listed in Table 6I.

TABLE 6I
BLAST results for NOV6

| | | | Identity | Positives | |
|---|---|---|---|---|---|
| Gene Index/Identifier | Protein/Organism | Length (aa) | (%) | (%) | Expect |
| ref\|XP_061214. | similar to lipase A | 395 | 327/397 | 327/397 | 0.0 |
| 1\| (XM_061214) | precursor; Lipase A, | | (82%) | (82%) | |
| | lysosomal acid, | | | | |
| | cholesterol esterase | | | | |
| | (H. sapiens) [Homo | | | | |
| | sapiens] | | | | |
| Ref\|XP_061221. | similar to lipase A | 395 | 327/397 | 327/397 | 0.0 |
| 1\| (XM_061221) | precursor; Lipase A, | | (82%) | (82%) | |
| | lysosomal acid, | | | | |
| | cholesterol esterase | | | | |
| | (H. sapiens) [Homo | | | | |
| | sapiens] | | | | |
| ref\|XP_061221. | similar to lipase A | 351 | 307/373 | 307/373 | e−174 |
| 1\| (XM_061221) | precursor; Lipase A, | | (82%) | (82%) | |
| | lysosomal acid, | | | | |
| | cholesterol esterase | | | | |
| | (H. sapiens) [Homo | | | | |
| | sapiens] | | | | |
| ref\|NP_000226. | lipase A precursor; | 399 | 192/370 | 251/370 | e−107 |
| 1\| (NM_000235) | Lipase A, lysosomal | | (51%) | (66%) | |
| | acid, cholesterol | | | | |
| | esterase [Homo | | | | |
| | sapiens] | | | | |
| pir\|\|S41408 | lysosomal acid lipase | 399 | 192/370 | 251/370 | e−107 |
| | (EC 3.1.1.—) / sterol | | (51%) | (66%) | |
| | esterase (EC 3.1.1.13) | | | | |
| | precursor - human | | | | |

The homology of these sequences is shown graphically in the ClustalW analysis shown in Table 6J.

[image]

[image]

Table 6K list the domain description from DOMAIN analysis results against NOV6. This indicates that the NOV6 sequence has properties similar to those of other proteins known to contain these domains.

TABLE 6K
Domain Analysis of NOV6

gnl|Pfam|pfam00561, abhydrolase, alpha/beta hydrolase fold catalytic domain
(SEQ ID NO:95)
Length = 226 residues, 96.0% aligned
Score = 226 bits (155), Expect = 1e−11

| | | | |
|---|---|---|---|
| Query: | 111 | YDVWMGNSRGNTWSRRHKTLSETDEKFWAFGFDEMAKYDLPGVIDFIVNKTGQEKLYFIG | 170 |
| | | +\|\| + + \|\| \| + \|\|++\|+ ++ +++ \| +\|+ +\| | |
| Sbjct: | 1 | FDVIL9DLRGFGQSSPSDLAE--------YRFDDLAED-----LEALLDALGLDKVILVG | 47 |
| Query: | 171 | HSLGTTIGFVAPSTMPELAQRIKMNFALGPTISFKYPTGIFTRFFLLPNSIIKAVFGTKC | 230 |
| | | \|\|+\| \| + \|\| \|+\| + + +\| \| + | |
| Sbjct: | 48 | HSMGGAIAAAYAAKYPE---RVKALVLVSAPHPALLSSRLFPRNLFGLLLANFRNRLLRS | 104 |
| Query: | 231 | FFLEDKKTKIASTKICNNKILWLICSEFMSLWAGSNKKNMNQLYHSDEFRAYDWGNDADN | 290 |
| | | + \| +\|+ +\| + ++ \| \| \| | |
| Sbjct: | 105 | VEALLGRA---------LKQFFLLQRPLVS--DFLKQFELSSLIRFGEDDGGDGLL--WV | 151 |
| Query: | 291 | MKHYNQSEPPIYDLTAMKVPTAIWAGGHDVLVTPQDVARILPQIKSLHYFKLLPDWNHFD | 350 |
| | | \|\| +\|\|\|\| + \| \| \|\| \| ++ + ++ \| \| | |
| Sbjct: | 152 | ALGKLLQWDVSADLKRIKVPTLVIWGDDDPLVPPDASEKLSALFPNAEVV-VIDDAGHLA | 210 |
| Query: | 351 | FVWCLDA | 357 |
| | | + + | |
| Sbjct: | 211 | QLEKPEE | 217 |

LIPB was assigned to chromosome 16 by study of somatic cell hybrids (Van Cong et al., 1980). Lysosomal acid lipase-A (LIPA) is the enzyme deficient in the presumably allelic Wolman disease and cholesterol ester storage disease. The distinct kinetic and physical properties of lipases A and B were defined by Warner et al. (1980). They stated that the natural substrate for LIPB is not known, and that it is not clear that LIPB is a lysosomal hydrolase. LIPA may serve an important role in cellular metabolism by releasing cholesterol. The liberated cholesterol suppresses further cholesterol synthesis and stimulates esterification of cholesterol within the cell. Lysosomal acid lipase (LIPA, or LAL), otherwise known as acid cholesteryl ester hydrolase, is coded for by a gene (LIPA) on chromosome 10.

Two major disorders, the severe infantile-onset Wolman disease and the milder late-onset cholesteryl ester storage disease (CESD), are seemingly caused by mutations in different parts of the LIPA gene. Wolman et al. (Pediatrics 28: 742-757,1961) described 3 sibs in whom involvement of the viscera was an important feature and death occurred at the age of about 3 months. Xanthomatous changes were observed in the liver, adrenal, spleen, lymph nodes, bone marrow, small intestine, lungs and thymus, and slight changes were found in the skin, retina, and central nervous system. The adrenals were calcified. Death was thought to be due to intestinal malabsorption resulting from involvement of the gut. The parents, Persian Jews, were cousins. Lipids in the plasma were normal or moderately elevated. Several features suggested that the entity is distinct from hypercholesterolemia and the hyperlipidemias (q.v.). Three cases, the first from the U.S.A., were reported by Crocker et al. (Pediatrics 35: 627-640, 1965), who gave no information on ethnicity. The relatively nonspecific clinical picture includes poor weight gain, vomiting, diarrhea, increasing hepatosplenomegaly with abdominal protuberance, and death by nutritional failure by 2 to 4 months of age. Foam cells are found in bone marrow and vacuolated lymphocytes in peripheral blood, as in Niemann-Pick disease (257200). Diffuse punctate calcification of the adrenals is typical. Disseminated foam cell infiltration is found in many organs. Great increases in cholesterol are found in the organs. Konno et al. (Tohoku J. Exp. Med. 90: 375-389, 1966) reported a Japanese family with 3 affected sibs. Spiegel-Adolf et al. (Confin. Neurol. 28: 399-406, 1966) reported 3 affected sibs in an American family.

Patrick and Lake (Nature 222: 1067-1068, 1969) demonstrated deficiency of an acid lipase (cholesteryl ester hydrolase; EC 3.1.1.13 ) which apparently leads to the progressive accumulation of triglycerides and cholesterol esters in lysosomes in the tissues of affected persons. Lough et al. (Arch. Path. 89: 103-110, 1970) described an affected infant of Greek ancestry in whom calcified adrenals were demonstrated on the 5th day of life. Young and Patrick (Arch. Dis. Child. 45: 664-668, 1970) commented on the existence of cases with the same biochemical and histologic changes as in the acute infantile form but with later onset and a much less fulminant course. One of their cases was alive and well at age 8 years, showing no clinical abnormality other than moderate hepatomegaly. The same enzyme is deficient in all these cases. Hence, they suggested the term ‘acid lipase deficiency’ for the whole group, with Wolman disease as the designation for the acute infantile form. Burton and Reed (Am. J. Hum. Genet. 33: 203-208, 1981) demonstrated material crossreacting with antibodies to acid lipase in fibroblasts of 3 patients with Wolman disease and 3 with cholesterol ester storage disease. Quantitation of the CRM showed normal levels in both cell types. Enzyme activity was reduced about 200-fold in Wolman disease fibroblasts and 50- to 100-fold in cholesterol ester storage disease cells. Presumably, cholesterol ester storage disease is a disorder allelic to Wolman disease (Assmann and Fredrickson: Acid lipase deficiency (Wolman's disease and cholesteryl ester storage disease).In: Stanbury, J. B.; Wyngaarden, J. B.; Fredrickson, D. S.; Goldstein, J. L.; Brown, M. S.: Metabolic Basis of Inherited Disease. New York: McGraw-Hill (pub.) (5th ed.) 1983. Pp. 803-819.), but experiments such as cell-fusion studies have not, to my knowledge, been done to establish this as fact. Supporting the allelic nature of Wolman and cholesteryl ester storage diseases is the occurrence of possible genetic compounds, i.e., cases of intermediate severity (Schmitz and Assmann: Acid lipase deficiency: Wolman disease and cholesteryl ester storage disease.In: Scriver, C. R.; Beaudet, A. L.; Sly, W. S.; Valle, D. The Metabolic Basis of Inherited Disease. New York: McGraw-Hill (pub.) (6th ed.) 1989. Pp. 1623-1644.).

In both Wolman disease and cholesteryl ester storage disease, Chatterjee et al. (Clin. Genet. 29: 360-368, 1986) demonstrated that renal tubular cells shed in the urine are laden with cholesteryl esters and triacylglycerol and that LIPA is lacking in these cells. Yoshida and Kuriyama (Lab. Animal Sci. 40: 486-489, 1990) described lysosomal acid lipase deficiency in rats. Roytta et al. (Clin. Genet. 42: 1-7, 1992) reported the case of an affected 1-month-old girl on the Aland Islands, the first published Scandinavian example of Wolman disease. Skin biopsy showed cytoplasmic accumulations identical to those noted in 2 Aland Islander sibs who died at the age of about 3 months during the 1950s. Genealogic analyses showed that the 2 families had ancestors from the same restricted area as well as common ancestors during the 17th century. The parents of the 2 affected sibs were born on a small island and were related to each other ‘in many different ways.’ Schiff et al. (Am. J. Med. 44: 538-546, 1968) described cholesterol ester storage disease of the liver in teenage brother and sister whose livers were orange in color. Four younger sibs showed milder changes. The parents were not known to be related. Tissue accumulation of cholesterol esters and triglycerides occurs in both this disease and Wolman disease. The chemical and enzymatic abnormalities are similar. The marked difference in phenotypic expression is unexplained but is comparable to the difference between Hurler and Scheie syndromes, the late infantile and adult forms of metachromatic leukodystrophy, and the classic and visceral forms (A and B) of Niemann-Pick disease. Each of these is presumably a pair of allelic disorders.

In contrast to Wolman disease, cholesterol ester storage disease is relatively benign; however, in 1 sibship 3 sisters died of acute hepatic failure at the ages of 7, 9, and 17 years (Beaudet et al., J. Pediat. 90: 910-914, 1977). Accumulation of neutral fats and cholesterol esters in the arteries predispose affected persons to atherosclerosis. Hypercholesterolemia is common. Massive hepatomegaly and hepatic fibrosis may lead to esophageal varices. Lysosomal acid lipase A, the enzyme deficient in both Wolman disease and this disorder, is one of three acid lipase isozymes. The role of lipases B and C is unknown. Besley et al. (Clin. Genet. 26: 195-203, 1984) reported the first patient observed in Ireland. Then aged 39, with hepatomegaly and sea-blue histiocytes in the bone marrow, the patient had suffered from recurring periods of general malaise and diarrhea since age 21. Desai et al. (Am. J. Med. Genet. 26: 689-698, 1987) made the prenatal diagnosis of this disorder by demonstration of deficient lysosomal acid lipase activity in cultured amniocytes from an at-risk fetus. The findings in the affected fetus at 17 weeks were described. Massive lysosomal cholesterol and lipid accumulation was demonstrated in fetal hepatocytes, adrenal cells, and syncytiotrophoblasts. Of particular note was the finding of extensive necrosis in the fetal adrenal glands. Necrosis of the adrenal may precede the calcification observed later in these patients. Cagle et al. (Am. J. Med. Genet. 24: 711-722, 1986) concluded that patients with CESD are at risk for the development of pulmonary hypertension. Such was recognized in a 15-year-old patient who died at age 18. Di Bisceglie et al. (Hepatology 11: 764-772, 1990) could demonstrate no significant changes in serum lipoprotein concentrations or liver histopathology after 12 months or more of treatment with lovastatin, a cholesterol-lowering agent. Yokoyama and McCoy (J. Inherit. Metab. Dis. 15: 291-292, 1992) observed some improvement with combined cholestyramine and lovastatin therapy. Koch et al. (Cytogenet. Cell Genet. 25: 174, 1979; Somat. Cell Genet. 7: 345-358, 1981) assigned lysosomal acid lipase A to chromosome 10 by human-Chinese hamster somatic cell hybrids. Judging from the close concordance with GOT1 (138180), these loci may be close together on the long arm of 10. Lipase A is encoded by chromosome 19 in mouse (Koch et al., 1981).

Soluble glutamate oxaloacetate transaminase (138180) is also on chromosome 10 q in man and 19 in mouse. By fluorescence in situ hybridization, Anderson et al. (Genomics 15: 245-247, 1993) mapped the LIPA locus to 10q23.2-q.23.3. It was clearly distinct from the locus for pancreatic lipase (246600) at 10q26. 1. Anderson and Sando (J. Biol. Chem. 266: 22479-22484, 1991) reported that the amino acid sequence of LAL as deduced from the 2.6-kb cDNA nucleotide sequence is 58% identical to that of human gastric lipase, which is involved in the preduodenal breakdown of ingested triglycerides. Aslanidis et al. (Genomics 20: 329-331, 1994) summarized the exon structure of the LIPA gene, which consists of 10 exons, together with the sizes of genomic EcoRI and SacI fragments hybridizing to each exon. The DNA sequence of the putative promoter region was presented. Anderson et al. (Proc. Nat. Acad. Sci. 91: 2718-2722, 1994) isolated and sequenced the gene for LIPA. They found that it is spread over 36 kb of genomic DNA. The 5-prime flanking region is GC-rich and has characteristics of a ‘housekeeping’ gene promoter. Aslanidis et al. (Genomics 33: 85-93,1996) provided evidence that a strikingly more severe course of Wolman disease is caused by genetic defects of LAL that leave no residual enzyme activity. In a CESD patient, a G-to-A transition at position −1 of the exon 8 splice donor site resulted in skipping of exon 8 in 97% of the mRNA originating from this allele, while 3% was spliced correctly, resulting in full-length LAL enzyme.

Pagani et al. (Hum. Molec. Genet. 5: 1611-1617,1996) described the molecular basis of CESD in 3 patients. They identified mutations by sequence analysis of LAL cDNA and genomic DNA. The role of the mutations as the direct cause of the disease was confirmed by measuring the LAL enzymatic activity of extracts from cells transfected with LAL mutants. The 3 CESD patients were found to be compound heterozygotes. Pagani et al. (1996) identified 3 different missense mutations, 2 splicing defects, and a null allele. Du et al. (Hum. Molec. Genet. 7: 1347-1354, 1998) produced a mouse model of lysosomal acid lipase deficiency by a null mutation produced by targeting disruption of the mouse gene. Homozygous knockout mice produced no Lip1 mRNA, protein, or enzyme activity. The homozygous deficient mice were born in mendelian ratios, were normal appearing at birth, and followed normal development into adulthood. However, massive accumulation of triglycerides and cholesteryl esters occurred in several organs. By 21 days, the liver developed a yellow-orange color and was up to 2 times larger than normal. The accumulated cholesteryl esters and triglycerides were approximately 30-fold greater than normal. The heterozygous mice had approximately 50% of normal enzyme activity and did not show lipid accumulation. Male and female homozygous deficient mice were fertile and could be bred to produce progeny. This mouse model is the phenotypic model of human CESD and a biochemical and histopathologic mimic of human Wolman disease. ALLELIC VARIANTS (selected examples) 0.0001 CHOLESTERYL ESTER STORAGE DISEASE [LIPA, LEU179PRO]

In a family with 2 children affected with CESD, Maslen and Illingworth (m. J. Hum. Genet. 53 (suppl.): A926, 1993) found compound heterozygosity for a 72-bp deletion corresponding to amino acids 254-277 in the allele inherited from the father, and a T-to-C transition at position 639 that resulted in substitution of a proline for leucine at position 179 in the allele inherited from the mother. 0.0006 WOLMAN DISEASE [LIPA, TYR22TER] In a Japanese patient with Wolman disease, Fujiyama et al. (Hum. Mutat. 8: 377-380, 1996) identified a tyr22-to-ter mutation of the LIPA gene. The female patient had an umbilical cord herniation at birth. At about 30 days after birth, she showed abdominal distention with hepatosplenomegaly and frequent episodes of diarrhea and vomiting. Abdominal computed tomography revealed massive hepatosplenomegaly and enlargement of the adrenal glands with calcification. Anemia and hepatic failure progressed rapidly and she died at age 114 days. The parents were first cousins. An older sister had died with similar symptoms 80 days after birth.

The protein similarity information, expression pattern, cellular localization, and map location for the NOV6 protein and nucleic acid disclosed herein suggest that the NOV6 lysosomal acid lipase-like proteins may have important structural and/or physiological functions characteristic of the Lysosomal Acid Lipase family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from: severe infantile-onset Wolman disease and the milder late-onset cholesteryl ester storage disease (CESD), obesity, diabetes, Von Hippel-Lindau (VHL) syndrome, and pancreatitis as well as other diseases, disorders and conditions. The novel nucleic acid encoding the polydom-like protein of the invention, or fragments thereof, are useful in diagnostic applications, wherein the presence or amount of the nucleic acid or the protein are to be assessed.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV6 protein has multiple hydrophilic regions, each of which can be used as an immunogen. In one embodiment, a contemplated NOV6 epitope is from about amino acids 40 to 60. In another embodiment, a contemplated NOV6 epitope is from about amino acids 70 to 80. In other specific embodiments, contemplated NOV6 epitopes are from about amino acids 90 to 95, 110 to 140, 150 to 152, 155 to 157, 240 to 250, 270 to 280, 310 to 315 and 320 to 325.

NOV7

A disclosed NOV7 nucleic acid (alternatively referred to herein as CG56140-01) encodes a novel tryptase 4-like protein and includes the 1608 nucleotide sequence (SEQ ID NO: 33) shown in Table 7A. An open reading frame for the mature protein was identified beginning with an ATG codon at nucleotides 1-3 and ending with a TGA codon at nucleotides 1279-1281. Putative untranslated regions are underlined in Table 7A, and the start and stop codons are in bold letters.

TABLE 7A
NOV7 Nucleotide Sequence

(SEQ ID NO:33)

ATGGGCGCGCGCGGGGCGCTGCTGCTGGCGCTGCTGCTGGCTCGGGCTGGACTCGGGAAGCCGGAGGCCTGCGGCCACCGGGAA
ATTCACGCGCTGGTGGCGGGCGGAGTGGAGTCCGCGCGCGGGCGCTGGCCATGGCAGGCCAGCCTGCGCCTGAGGAGACGCCAC
CGATGTGGAGGGAGCCTGCTCAGCCGCCGCTGGGTGCTCTCGGCTGCGCACTGCTTCCAAAACAGTCGTTACAAAGTCCAGGAC
ATCATTGTGAACCCTCACCCACTTGGGGTTTTACGCAATGACATTGCCCTGCTGAGACTGGCCTCTTCTGTCACCTACAATGCG
TACATCCAGCCCATTTGCATCGAGTCTTCCACCTTCAACTTCGTGCACCGGCCGGACTGCTGGGTGACCGGCTGGCGGTTAATC
AGCCCCAGTGGCACACCTCTGCCACCTCCTTACAACCTCCGGGAAGCACAGGTCACCATCTTAAACAACACCAGGTGTAATTAC
CTGTTTGAACAGCCCTCTAGCCGTAGTATGATCTCCGATTCCATGTTTTGTGCTGGTGCTGAGGATGGCAGTGTAGACACCTGC
AAAGGTGACTCAGGTGGACCCTTGGTCTGTGACAAGGATGGACTGTGGTATCAGGTTGGAATCGTGAGCTGGGGAATGGACTGC
GGTCAACCCAATCGGCCTGGTGTCTACACCAACATCAGTGTGTACTTCCACTGGATCCGGAGGGTGATGTCCCACAGTACACCA
AGGCCAAACCCCTCCCCAGCTGTTGCTGCTCCTTGCCCTGCTGTGGGCTCCCTGACTCCTGCAGCCATTCTGAGTGCACCAGAA
ACTGTGAGGCTGCAGTGGGGACCACAGTATTGGCTCACCTCCTCTGGGCTGTCGGCGCTTCAGGGACACCGTTGGGACTGCCTG
CTGGATCAGATTCCCGCCCCTTTTGTCTCGTTTGCTAATAAATACGTGTGCATGTTCAAGCTGATCCCTTACAGAGCTTTCTGT
GGACCTAAGGGGTTTCGTGGACAACTCCCTCCTCTTCACTCATGTCCAGTCCAGGCCAAGACCCCACCTGAACTCCTAAATTGT
TATCCAGGTTTTTGTTGCGAACAGCAGCACCCTCTGGTTATTTCCATCGGAAAGATAATTGATGGAAGAGCAGTAGTACTTCAG
TGTGTCAGAGGGGTGGGAAGACATGGATTGGGGGTGCCATGGAGGAAATGCTCCCAGTGCTCCCATCCTAGGGTTCCCAATCAC
ACAAATGCCAGATGTTCCTGATCTTATTTTGGTCACTCCAATGGTTGACCTAAAACCACGACATQGGTGCGGTAGTTTATCTGG
AAGGTGATCCCAGGAAGCAAAGATGAGAAAGTGCAGAAACCAAGGCAGGAAAGGCACAAATGCCAATGAATTTGCTCAAACTGG
GAGAAATTGGGCCACAATCCTGTGGGGGCTTCAGCATTCGCCCACTGAGGACAAGGACACCAGGAGCGAGTGTTTCTCTCTTCC
CCCTGATGCCACAAGTTGAGGGAGGTCCTTCGGGCATGGTATGATGCCCCAGAGCTCTAGCAGCCCCTAGGTGAGCCTGTGGGC
ACCCAGGCAGCT

The nucleic acid sequence of NOV7 maps to chromosome 16 and invention has 587 of 853 bases (68%) identical to a gb:GENBANK-ID:ABO31329|acc:AB031329.1 mRNA from Homo sapiens (Homo sapiens esp-1 mRNA for eosinophil serine protease, complete cds) (E=5.7e⁻⁵⁸).

The NOV7 polypeptide (SEQ ID NO: 34) is 426 amino acid residues in length and is presented using the one-letter amino acid code in Table 7B. The SignalP, Psort and/or Hydropathy results predict that NOV7 has a signal peptide and is likely to be localized lysosome (lumen) with a certainty of 0.5500. In alternative embodiments, a NOV7 polypeptide is located to the outside of the cell with a certainty of 0.3700, the plasma membrane with a certainty of 0.1900, or the endoplasmic reticulum (membrane) with a certainty of 0.1000. The SignalP predicts a likely cleavage site for a NOV7 peptide between amino acid positions 19 and 20, i.e. at the dash in the sequence GLG-KP

TABLE 7B
Encoded NOV7 Protein Sequence

(SEQ ID NO:34)

MGARGALLLALLLARAGLGKPEACGHREIHALVAGGVESARGRWPWQASLRLRRRHRCGGSLLSRRWVLSAAHCFQNSRYK
VQDIIVNPDALGVLRNDIALLRLASSVTYNAYIQPICIESSTFNFVHRPDCWVTGWGLISPSGTPLPPPYNLREAQVTILN
NTRCNYLFEQPSSRSMIWDSMFCAGAEDGSVDTCKGDSGGPLVCDKDGLWYQVGIVSWGMDCGQPNRPGVYTNISVYFHWI
RRVNSHSTPRPNPSPAVAAPCPAVGSLTPAAILSAPETVRLQWCPQYWLTSSGLWALQGQGWDCLLDQIPAPFVSFANKYV
CMFKLMPYRAFCGPKGFRGQLPPLHSCPVQAKTPPELLNCYPGFCCEQQHPLVISIGKIIDGRAVVLQCVRGVGRHGLGVP
WRKCSQCSHPRVPNHTNARCS

The NOV7 amino acid sequence has 105 of 199 amino acid residues (52%) identical to, and 140 of 199 amino acid residues (70%) similar to, the 305 amino acid residue ptnr:SPTREMBL-ACC:Q9JHJ7 protein from Mus musculus (Mouse) (TRYPTASE 4) (E=6.5e⁻⁷³)

NOV7 is expressed in at least the following tissues: pancreas. This information was derived by determining the tissue sources of the sequences that were included in the invention including but not limited to SeqCalling sources, Public EST sources, Literature sources, and/or RACE sources.

NOV7 also has homology to the amino acid sequences shown in the BLASTP data listed in Table 7C.

TABLE 7C
BLAST results for NOV7

| Gene Index/ | Protein/ | | | | |
|---|---|---|---|---|---|
| Identifier | Organism | Length (aa) | Identity (%) | Positives (%) | Expect |
| gi\|15823587\|dbj\|B | testis serine | 322 | 143/252 | 179/252 | 7e−77 |
| AB68561.1\| | protease-1 [Mus | | (56%) | (70%) | |
| (AB049453) | musculus] | | | | |
| gi\|12855280\|dbj\|B | putative [Mus | 282 | 143/252 | 179/252 | 3e−76 |
| AB30277.1\| | musculus] | | (56%) | (70%) | |
| (AK016509) | | | | | |
| gi\|11055972\|ref\|N | tryptase 4; | 324 | 125/251 | 167/251 | 1e−67 |
| P_065233.2\| | protease, | | (49%) | (55%) | |
| (NM_020487) | serine, 21 | | | | |
| | (testisin) [Mus | | | | |
| | musculus] | | | | |
| gi\|12839280\|dbj\|B | putative [Mus | 296 | 124/251 | 166/251 | 1e−66 |
| AB24495.1\| | musculus] | | (49%) | (65%) | |
| (AK006271) | | | | | |
| gi\|8777606\|gb\|AAF | testisin [Homo | 312 | 126/255 | 163/255 | 2e−63 |
| 79020.1\| | sapiens] | | (49%) | (63%) | |
| (AF058301) | | | | | |

The homology of these sequences is shown graphically in the ClustalW analysis shown in Table 7D.

[image]

[image]

Tables 7E and 7F list the domain description from DOMAIN analysis results against NOV7. This indicates that the NOV7 sequence has properties similar to those of other proteins known to contain these domains.

TABLE 7E
Domain Analysis of NOV7

gnL|Smart|smart00020, Tryp_SPc, Trypsin-like serine protease (SEQ ID NO:101)
Length = 230 residues, 99.6% aligned
Score = 220 bits (560), Expect = 1e−58

| | | | |
|---|---|---|---|
| Query: | 33 | VAGGVESARGRWPWQASLRLRR-RHRCGGSLLSRRWVLSAAHCFQNSR------------ | 79 |
| | | + \|\| \|+ \| +\|\|\| \|\|+ \| \|\| \|\|\|\|\|+\| \|\|\|\|+\|\|\|\| \| | |
| Sbjct: | 2 | IVGGSEANIGSFPWQVSLQYRGGRHFCGGSLISPRWVLTAAHCVYGSAPSSIRVRLGSHD | 61 |
| Query: | 80 | ---------YKVQDIIVNPDALGV-LRNDIALLRLASSVTYNAYIQPICIESSTFNFVHR | 129 |
| | | \|\| +\|\|+\|+ \|\|\|\|\|\|+\|+ \|\| + ++\|\|\|+ \|\| +\| | |
| Sbjct: | 62 | LSSGEETQTVKVSKVIVHPNYNPSTYDNDIALLKLSEPVTLSDTVRPICLPSSGYNVPAG | 121 |
| Query: | 130 | PDCWVTGWGLISPSGTPLPPPYNLREAQVTILNNTRCNYLFEQPSSRSMIWDSMFCAGAE | 189 |
| | | \| \|+\|\|\| \| \| \|\| \|+\| \| \|++\| \| \| \| \|+\| \|\|\| | |
| Sbjct: | 122 | TTCTVSGWGRTSESSGSLPD--TLQEVNVPIVSNATCR---RAYSGCPAITDNMLCAGGL | 176 |
| Query: | 190 | DGSVDTCKGDSGGPLVCDKDGLWYQVGIVSWGMD-CGQPNRPGVYTNISVYFHWI | 243 |
| | | +\| \| \|+\|\|\|\|\|\|\|\|\| \| \| \|\|\|\|\|\|\| \| +\|\|+\|\|\|\|\| +\| \| \|\| | |
| Sbjct: | 177 | EGGKDACQGDSGGPLVC-NDPRWVLVGIVSWGSYGCARPNKPGVYTRVSSYLDWI | 230 |

TABLE 7F
Domain Analysis of NOV7

gnl|Pfam|pfam00089, trypsin, Trypsin (SEQ ID NO:102)
Length = 217 residues, 99.1% aligned
Score = 172 bits (436), Expect = 3e−44

| | | | |
|---|---|---|---|
| Query: | 35 | GGVESARGRWPWQASLRLRRRERCGGSLLSRRWVLSAAHCFQNS---------------- | 78 |
| | | \|\| \|+ \| +\|\|\| \|\|++ \| \|\|\|\|\|+\| \|\|\|+\|\|\|\| + | |
| Sbjct: | 3 | GGREAQAGSFPWQVSLQVSSGHPCGGSLISENWVLTAAHCVSGASSVRVVLGEHNLGTTE | 62 |
| Query: | 79 | ----RYKVQDIIVNPDALGVLRNDIALLRLASSVTYNAYIQPICIESSTFNFVHRPDCWV | 134 |
| | | ++ \|+ \|\|\|+\|+ \|\|\|\|\|\|+\| \| \|\| ++\|\|\|+ \|++ + \| \| | |
| Sbjct: | 63 | GTEQKFDVKKIIVHPNY-NPDTNDIALLKLKSPVTLGDTVRPICLPSASSDLPVGTTCSV | 121 |
| Query: | 135 | TGWGLISFSGTPLPPPYNLREAQVTILNNTRCNYLFEQPSSRSMIWDSMFCAGAEDGSVD | 194 |
| | | +\|\|\| \| \|+\| \| \|++ \| + + + \|+\| \|\|\|\| \| \| | |
| Sbjct: | 122 | SGWGRTKNLG----TSDTLQEVVVPIVSRETC-----RSAYGGTVTDTMICAGALGG-KD | 171 |
| Query: | 195 | TCKGDSGGPLVCDKDGLWYQVGIVSWGMDCGQPNRPGVYTNISVYFHWI | 243 |
| | | \|+\|\|\|\|\|\|\|\|\| \|\|\|\|\|\|\| \| \| \|\|\|\|\| +\| \| \|\| | |
| Sbjct: | 172 | ACQGDSGGPLVCSDG---ELVGIVSWGYGCAVGNYPGVYTRVSRYLDWI | 217 |

Human tryptase is a structurally unique and mast cell specific trypsin-like serine protease. Recent biological and immunological investigations have implicated tryptase as a mediator in the pathology of numerous allergic and inflammatory conditions including rhinitis, conjunctivitis, and most notably asthma. A growing body of data further implicates tryptase in certain gastrointestinal, dermatological, and cardiovascular disorders as well. The recent availability of potent, and selective tryptase inhibitors, though, has facilitated the validation of this protease as an important therapeutic target as well. Herein, we describe the design and potency of four classes of selective tryptase inhibitors, of which the first three types are synthetic and the fourth is natural in origin: 1) peptidic inhibitors (e.g., APC-366), 2) dibasic inhibitors (i.e., pentamidine-like), 3) Zn(2+)-mediated inhibitors (i.e., BABIM-like), and 4) heparin antagonists (e.g., lactoferrin). These inhibitors have been tested in the airways and skin of allergic sheep. Aerosol administration of tryptase inhibitors from each structural class 30 minutes before, and 4 hours and 24 hours after allergen challenge, abolishes late phase bronchoconstriction and airway hyperresponsiveness in a dose-dependent manner. Moreover, intradermal injection of APC-366 blocks the cutaneous response to antigen. These studies provide the essential proof-of-concept for the further pursuit of tryptase inhibitors for the treatment of asthma, and perhaps other allergic diseases. Results from clinical studies with the first generation tryptase inhibitor APC-366, currently in phase II trials for the treatment of asthma, provide additional support for a pathological role for tryptase in this disease. Notable advances in the area of tryptase inhibitor design at Axys Pharmaceuticals, Inc. include a novel, zinc-mediated, serine protease inhibitor technology (described herein), and the discovery of a unique class of extremely potent and selective dibasic tryptase inhibitors. Independently, an X-ray crystal structure of active tryptase tetramer complexed with 4-amidinophenyl pyruvic acid has been reported. It is anticipated that these discoveries will further accelerate the design of structurally novel tryptase inhibitors as well as the development of new drugs for the treatment of mast cell tryptase-mediated disorders.

The protein similarity information, expression pattern, and map location for the NOV7 protein and nucleic acid disclosed herein suggest that it may have important structural and/or physiological functions characteristic of the Serine Protease family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: diabetes, Von Hippel-Lindau (VHL) syndrome, pancreatitis, obesity, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, transplantation, fertility, endometriosis, Hirschsprung's disease, Crohn's disease, appendicitis and other diseases, disorders and conditions of the like.

The novel nucleic acid encoding the novel tryptase-4 protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV7 protein has multiple hydrophilic regions, each of which can be used as an immunogen. In one embodiment, a contemplated NOV7 epitope is from about amino acids 10 to 15. In another embodiment, a contemplated NOV7 epitope is from about amino acids 20 to 25. In other specific embodiments, contemplated NOV7 epitopes are from about amino acids 40 to 60, 70 to 80, 80 to 85, 120 to 160, 180 to 200, 220 to 260, 280 to 300, 340 to 360 and 420 to 430.

NOV8

A disclosed NOV8 nucleic acid (designated as CuraGen Acc. No. CG56134-01), encodes a novel P450-like protein and includes the 1539 nucleotide sequence (SEQ ID NO: 35) shown in Table 8A. An open reading frame for the mature protein was identified beginning with an ATG codon at nucleotides 1-3 and ending with a TAA codon at nucleotides 1537-1539. The start and stop codons are in bold letters in Table 8A.

TABLE 8A
NOV8 Nucleotide Sequence

(SEQ ID NO:35)

ATGGCGGGGCTCTGGCTGGGGCTCGTGTGGCAGAACCTGCTGCTGTCGGGCGCGGCGAGTGCCGTTTCCCTGQCCGGCGCCAGT
CTGGTCCTGAGCCTGCTGCAGAQGGTGGCGAGCTACGCGCGGAAATGGCAGCAGATGCGGCCCATCCCCACGGTGGCCCGCGCC
TACCCACTGGTGGGCCACGCGCTGCTGATGAAGCCGGACGGGCGAGAATTTTTTCACCAGATCATTGAGTACACAQAGGAATAC
CGCCACATGCCGCTGCTGAAGCTCTGGGTCGGGCCAGTGCCCATGGTGGCCCTTTATAATGCAGAAAATGTGGAGAATCCTGGC
TCAGAGAAGAGAGCAAGAAGGGCAGACAGCATTTCTGCCGCTGTTGGCCTAGTGTTAATAGAAGTTCGTGTGGTGGATGCAGAT
CGAGATCTGTCCAGAGTAGGGGACTTGAGCAAGAAGCCTGATATATTTTTTGTAACCACATATTTTATTTCTACTACTGCAAAC
AAATGGCGCTCCAGGAGAAAGATGTTAACACCCACTTTCCATTTTACCATTCTGGAACATTTCTTAGATATCATCAATGAACAA
GCAAATATATTGGTTAAGAAACTTGAAAAACACATTAACCAAGAAGCATTTAACTGCTTTTTTTACATCACTCTTTGTCCCTTA
GATATCATCTGTGAGAAAATGGCCCAAACAGGAAATCACACTCCACTGGQAAGGCAAATGGGGGGACGGGAAAGGGTGACGGGC
TCTTCAGCGCGGTTCTACGACCQCACTGGCCTTCTGAGGAGCAGCAGCCACGCCCAGCGCTGTGAGTGGGCCAGGCATCCAGCA
ACTGCCCAGGGACGCGAAGGGAAGGAAGAACAGGAACAGGGAGTAGAAGTGGACCGTACAAGAGAAGAGGGTAAAGCCAGGAAG
AAGAATTCTGAAATATACAAGGACAAAGCAGGATCTATGGGGAAGAATATTGGTGCTCAAAGTAATGATGATTCCGAGTATGTC
CGTGCAGTTTATAGAATGAGTGAGATGATATTTCGAAGAATAAAGATGCCCTGGCTTTGGCTTGATCTCTGGTACCTTATGTTT
AAAGAAGGATGGGAACACAAAAAGAGCCTTCAGATCCTACATACTTTTACCAACAGTGTCATCGCTGAACGGGCCAATGAAATG
AACGCCAATGAAGACTGTAGAGGTGATGGCAGGGGCTCTGCCCCCTCCAAAAATAAACGCAGGGCCTTTCTTGACTTGCTTTTA
AGTGTGACTGATGACGAAGGGAACAGGCTAAGTCATGAAGATATTCGAGAAGAAGTTGACACCTTCATGTTTGAGGCTGGTGCA
GGCTGCAACTGCCCAGGTTCCAGCTGTGAGCTAAAAGTGGGCGTCCTCCCCTGCTCCACCAGTGTGCCTCGGTGCTTTACGTTT
GCCTTATCATGCTTTCTGCAGTTGGCAGATGAAATGAAATCGGAAGTTCAGCAGACTCCTCTTATGCATTTGGATCAGGCTTCC
GCTCATAAATTCAAGGAAAGCTATTAA

The nucleic acid sequence of NOV8 maps to chromosome 4 and has 158 of 252 bases (62%) identical to a gb:GENBANK-ID:AF251548|acc:AF251548.1 mRNA from Tribolium castaneum (Tribolium castaneum cytochrome P450 monooxigenase CYP4Q4 (CYP4Q4) mRNA, complete cds) (E=1.5e⁻⁰⁶).

The NOV8 polypeptide (SEQ ID NO: 36) is 512 amino acid residues in length and is presented using the one-letter amino acid code in Table 8B. The SignalP, Psort and/or Hydropathy results predict that NOV8 has a signal peptide and is likely to be localized to the plasma membrane with a certainty of 0.6000. In alternative embodiments, a NOV8 polypeptide is located to the Golgi body with a certainty of 0.4000, the mitochondrial intermembrane space with a certainty of 0.3131, or the endoplasmic reticulum (membrane) with a certainty of 0.3000. The SignalP predicts a likely cleavage site for a NOV8 peptide between amino acid positions 39 and 40, i.e. at the dash in the sequence VAS-YA

TABLE 8B
Encoded NOV8 Protein Sequence

(SEQ ID NO:36)

MAGLWLGLVWQKLLLWGAASAVSLAGASLVLSLLQRVASYARKWQQMRPIPTVARAYPLVGHALLMKPDGREFFQQIIEYT
EEYRHMPLLKLWVGPVPMVALYNAENVENPGSEKRARRADRISAAVGLVLIEVGVVDADGDLSRVGDLSKKPDIFFVTTYF
ISSTGNKWRSRRKMLTPTFHFTILEDFLDIMNEQANILVKKLEKUINQEAFNCFFYITLCALDIICEKMAQTGNHTPLGRQ
MGGRERVTGSSARFYDRTGLLRSSSHAQGCEWGRHGATAQGGEGKEEQEQGVEVDRTREEGKGRKKNSEIYKDKAGSMGKN
IGAQSNDDSEYVRAVYRMSEMIFRRIKMPWLWLDLWYLMFKEGWEHKKSLQILHTFTNSVIAERANEMNANEDCRGDGRGS
APSKNKRRAFLDLLLSVTDDEGNRLSHEDIREEVDTFMFEAGAGCNCPGSSCELKVGVLPCSTSVPRCFTFALSCFLQLAD
EMKSEVQQTPLMHLDQASAHKFKESY

The NOV8 amino acid sequence has 57 of 131 amino acid residues (43%) identical to, and 77 of 131 amino acid residues (58%) similar to, the 535 amino acid residue ptnr:SWISSNEW-ACC:Q9VA27 protein from Drosophila melanogaster (Fruit fly) (CYTOCHROME P450 4C3 (EC 1.14.-.-) (CYPIVC3)) (E=1.6e⁻⁴⁰).

NOV8 is expressed in at least the following tissues: liver, lung. This information was derived by determining the tissue sources of the sequences that were included in the invention including but not limited to SeqCalling sources, Public EST sources, Literature sources, and/or RACE sources.

NOV8 also has homology to the amino acid sequences shown in the BLASTP data listed in Table 8C.

TABLE 8C
BLAST results for NOV8

| Gene Index/ | Protein/ | | | | |
|---|---|---|---|---|---|
| Identifier | Organism | Length (aa) | Identity (%) | Positives (%) | Expect |
| gi\|12836111\|dbj\|BAB | putative [Mus | 525 | 123/219 | 149/219 | 7e−64 |
| 23507.1\| (AK004724) | musculus] | | (56%) | (67%) | |
| gi\|17542994\|ref\|NP_{—} | cytochrome | 511 | 66/193 | 97/193 | 1e−23 |
| 500637.1\| | P450 | | (34%) | (50%) | |
| (NM_068236) | [Caenorhabditis | | | | |
| | elegans] | | | | |
| gi\|17540954\|ref\|NP_{—} | Cytochrome | 467 | 67/193 | 96/193 | 2e−23 |
| 502152.1\| | P450 | | (34%) | (49%) | |
| (NM_069751) | [Caenorhabditis | | | | |
| | elegans] | | | | |
| gi\|17543882\|ref\|NP_{—} | cytochrome | 278 | 66/194 | 97/194 | 9e−23 |
| 502584.1\| | P450 | | (34%) | (49%) | |
| (NM_070183) | [Caenorhabditis | | | | |
| | elegans] | | | | |
| gi\|5263306\|gb\|AAC03 | family 4 | 501 | 56/127 | 78/127 | 5e−21 |
| 111.2\| (AF046010) | cytochrome | | (44%) | (61%) | |
| | P450 | | | | |
| | [Coptotermes | | | | |
| | acinaciformis] | | | | |

The homology of these sequences is shown graphically in the ClustalW analysis shown in Table 8D.

[image]

[image]

[image]

The P450 gene superfamily is a biologically diverse class of oxidase enzymes; members of the class are found in all organisms. P450 proteins are clinically and toxicologically important in humans; they are the principal enzymes in the metabolism of drugs and xenobiotic compounds, as well as in the synthesis of cholesterol, steroids and other lipids. Induction of some P450 genes can also be a risk factor for several types of cancer. This diversity of function is mirrored in the diversity of nucleotide and protein sequences; there are currently over 100 human P450 forms described. Allelic forms of many cytochrome P450 genes have been identified as causing quantitatively different rates of drug metabolism, and hence are important to consider in the development of safe and effective human pharmaceutical therapies. [reviewed in E. Tanaka, J Clinical Pharmacy & Therapeutics 24:323-329, 1999].

The protein similarity information, expression pattern, and map location for the NOV8 protein and nucleic acid disclosed herein suggest that it may have important structural and/or physiological functions characteristic of the P450 family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The novel nucleic acid encoding the P450-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV8 protein has multiple hydrophilic regions, each of which can be used as an immunogen. In one embodiment, a contemplated NOV8 epitope is from about amino acids 20 to 25. In another embodiment, a contemplated NOV8 epitope is from about amino acids 80 to 85. In other specific embodiments, contemplated NOV8 epitopes are from about amino acids 110 to 115, 140 to 145, 202 to 205, 220 to 320, 330 to 335, 380 to 405, 420 to 425 and 490 to 500.

NOV9

A disclosed NOV9 is nucleic acid (designated as CuraGen Acc. No. CG56207-01, encodes a novel mitsugumin29-like protein and includes the 813 nucleotide sequence (SEQ ID NO: 37) shown in Table 9A. An open reading frame for the mature protein was identified beginning at nucleotide 1 and ending with a TAA codon at nucleotides 805-807. Putative untranslated regions downstream from the termination codon are underlined in Table 9A, and the stop codon is in bold letters.

TABLE 9A
NOV9 Nucleotide Sequence (SEQ ID NO:37)

TCCTCACTCTCCCCCACTCCCGCACTTAATGTCTGCACCTGCGGGGAGTC
ACGGTTAGTTCTTGACTTTGGCCAGTTGAGGCCTTCTGACTCTCAAAGAG
GGTTCACTCTCTCTCAGCTCTTTGCTATTTTCGCCTTCGGGTCCTGTGGC
TCCTACAGCGGGGAGACAGGAGCAATGGTTCGCTGCAACAACGAAGCCAA
GGACGTGAGCTCCATCATCGTTGCATTTGGCTATCCCTTCAGGTTGCGCC
GGATCCAATATGAGATGCCCCTCTGCGATGAAGAGTCCAGCTCCAAGACC
ATGCACCTCATGGGGGACTTCTCTGCACCCGCCGAGTTCTTCGTGACCCT
TGGCATCTTTTCCTTCTTCTATACCATGGCTGCCCTAGTTATCTACCTGC
GCTTCCACAACCTCTACACAGAGAACAAACGCTTCCCGCTGGTGGACTTC
TGTGTGACTGTCTCCTTCACCTTCTTCTGGCTGGTAGCTGCAGCTGCCTG
GGGCAAGGGCCTGACCGATGTCAAGGGGGCCACACGACCATCCAGCTTGA
CAGCAGCCATGTCAGTGTGCCATGGAGAGGAAGCAGTGTGCAGTGCCGGG
GCCACGCCCTCTATGGGCCTGGCCAACATCTCCGTGCTCTTTGGCTTTAT
CAACTTCTTCCTGTGGGCCGGGAACTGTTGGTTTGTGTTCAAGGAGACCC
CGTGGCATGGACAGGGCCAGGGCCAGGACCAGGACCAGGACCAGGACCAG
GGCCAGGGTCCCAGCCAGGAGAGTGCAGCTGAGCAGGGAGCAGTGGAGAA
GCAGTAAGCAGCC

The nucleic acid sequence of NOV9 maps to chromosome 3 and has 606 of 676 bases (89%) identical to a gb:GENBANK-ID:AB004816|acc:AB004816.1 mRNA from Oryctolagus cuniculus (Oryctolagus cuniculus mRNA for mitsugumin29, complete cds) (E=2.0e⁻¹¹⁶)

The NOV9 polypeptide (SEQ ID NO: 37) is 268 amino acid residues in length and is presented using the one-letter amino acid code in Table 9B. The SignalP, Psort and/or Hydropathy results predict that NOV9 has a signal peptide and is likely to be localized to the plasma membrane with a certainty of 0.6000. In alternative embodiments, a NOV9 polypeptide is located to the Golgi body with a certainty of 0.4000, the endoplasmic reticulum (membrane) with a certainty of 0.3000, or the microbody (peroxisome) with a certainty of 0.3000. The SignalP predicts a likely cleavage site for a NOV9 peptide between amino acid positions 50 and 51, i.e. at the dash in the sequence SCG-SY.

TABLE 9B
Encoded NOV9 Protein Sequence (SEQ ID NO:38)

SSLSPTPALNVCTCGESRLVLDFGQLRPSDSQRGFTLSQLFAIFAFGSCG
SYSGETGAMVRCNNEAKDVSSIIVAFGYPFRLRRIQYEMPLCDEESSSKT
MHLMGDFSAPAEFFVTLGIFSFFYTMAALVIYLRFHNLYTENKRFPLVDF
CVTVSFTFFWLVAAAAWGKGLTDVKGATRPSSLTAAMSVCHGEEAVCSAG
ATPSMGLANISVLFGFINFFLWAGNCWFVFKETPWHGQGQGQDQDQDQDQ
GQGPSQESAAEQGAVEKQ

The NOV9 amino acid sequence has 223 of 268 amino acid residues (83%) identical to, and 235 of 268 amino acid residues (87%) similar to, the 264 amino acid residue ptnr:SPTREMBL-ACC:062646 protein from Oryctolagus cuniculus (Rabbit) (MITSUGUMIN29) (E=7.9e⁻¹¹⁵)

NOV9 is expressed in at least the following tissues: brain, skeletal muscle, heart. This information was derived by determining the tissue sources of the sequences that were included in the invention including but not limited to SeqCalling sources, Public EST sources, Literature sources, and/or RACE sources.

Possible small nucleotide polymorphisms (SNPs) found for NOV9are listed in Table 9C.

TABLE 9C
SNPs

| | Nucleotide | Base | Amino Acid | Base |
|---|---|---|---|---|
| Variant | Position | Change | Position | Change |
| 13375301 | 433 | T > C | 145 | Phe > Leu |
| 13375302 | 540 | A > G | NA | NA |

NOV9 also has homology to the amino acid sequences shown in the BLASTP data listed in Table 9D.

TABLE 9D
BLAST results for NOV9

| Gene Index/ | Protein/ | | | | |
|---|---|---|---|---|---|
| Identifier | Organism | Length (aa) | Identity (%) | Positives (%) | Expect |
| gi\|3077703\|dbj\|BAA2 | mitsugumin29 | 264 | 214/270 | 226/270 | e−110 |
| 5784.1\| (AB004816) | [Oryctolagus | | (79%) | (83%) | |
| | cuniculus] | | | | |
| gi\|6678874\|ref\|NP_0 | mitsugumin 29 | 264 | 201/232 | 209/232 | e−107 |
| 32622.1\| | [Mus musculus] | | (86%) | (89%) | |
| (NM_008596) | | | | | |
| gi\|2134413\|pir\|\|I50 | synaptophysin | 268 | 102/197 | 133/197 | 1e−52 |
| 720 | IIa - chicken | | (51%) | (66%) | |
| gi\|12836843\|dbj\|BAB | putative [Mus | 285 | 103/200 | 134/200 | 7e−52 |
| 23831.1\| (AK005132) | musculus] | | (51%) | (66%) | |
| gi\|13027428\|ref\|NP_{—} | Synaptoporin; | 265 | 103/197 | 132/197 | 2e−51 |
| 076464.1\| | synaptorin | | (52%) | (66%) | |
| (NM_023974) | [Rattus | | | | |
| | norvegicus] | | | | |

The homology of these sequences is shown graphically in the ClustalW analysis shown in Table 9E.

[image]

[image]

Synaptophysin and synaptoporin are related glycoproteins: they are the major integral membrane proteins of a certain class of small neurosecretory vesicles, although they may also be found in vesicles of various non-endocrine cells [1, 2]. The polypeptide chain spans the membrane four times and possibly acts as an ion or solute channel.

Recently mitsugumin29 unique to the triad junction in skeletal muscle was identified as a novel member of the synaptophysin family; the members of this family have four transmembrane segments and are distributed on intracellular vesicles. Mouse mitsugumin29 cDNA and genomic DNA containing the gene has been isolated and analyzed. The mitsugumin29 gene mapped to the mouse chromosome 3 F3-H2 is closely related to the synaptophysin gene in exon-intron organization, which indicates their intimate relationship in molecular evolution. RNA blot hybridization and immunoblot analysis revealed that mitsugumin29 is expressed abundantly in skeletal muscle and at lower levels in the kidney. Immunofluorescence microscopy demonstrated that mitsugumin29 exists specifically in cytoplasmic regions of the proximal and distal tubule cells in the kidney. The results obtained may suggest that mitsugumin29 is involved in the formation of specialized endoplasmic reticulum systems in skeletal muscle and renal tubule cells.

In skeletal muscle, excitation-contraction (E-C) coupling requires the conversion of the depolarization signal of the invaginated surface membrane, namely the transverse (T-) tubule, to Ca2+ release from the sarcoplasmic reticulum (SR). Signal transduction occurs at the junctional complex between the T-tubule and SR, designated as the triad junction, which contains two components essential for E-C coupling, namely the dihydropyridine receptor as the T-tubular voltage sensor and the ryanodine receptor as the SR Ca2+-release channel. However, functional expression of the two receptors seemed to constitute neither the signal-transduction system nor the junction between the surface and intracellular membranes in cultured cells, suggesting that some as-yet-unidentified molecules participate in both the machinery. In addition, the molecular basis of the formation of the triad junction is totally unknown. It is therefore important to examine the components localized to the triad junction. Here we report the identification using monoclonal antibody and primary structure by cDNA cloning of mitsugumin29, a novel transmembrane protein from the triad junction in skeletal muscle. This protein is homologous in amino acid sequence and shares characteristic structural features with the members of the synaptophysin family. The subcellular distribution and protein structure suggest that mitsugumin29 is involved in communication between the T-tubular and junctional SR membranes.

Physiological roles of the members of the synaptophysin family, carrying four transmembrane segments and being basically distributed on intracellular membranes including synaptic vesicles, have not been established yet. Recently, mitsugumin29 (MG29) was identified as a novel member of the synaptophysin family from skeletal muscle. MG29 is expressed in the junctional membrane complex between the cell surface transverse (T) tubule and the sarcoplasmic reticulum (SR), called the triad junction, where the depolarization signal is converted to Ca(2+) release from the SR. In this study, we examined biological functions of MG29 by generating knockout mice. The MG29-deficient mice exhibited normal health and reproduction but were slightly reduced in body weight. Ultrastructural abnormalities of the membranes around the triad junction were detected in skeletal muscle from the mutant mice, i.e., swollen T tubules, irregular SR structures, and partial misformation of triad junctions. In the mutant muscle, apparently normal tetanus tension was observed, whereas twitch tension was significantly reduced. Moreover, the mutant muscle showed faster decrease of twitch tension under Ca(2+)-free conditions. The morphological and functional abnormalities of the mutant muscle seem to be related to each other and indicate that MG29 is essential for both refinement of the membrane structures and effective excitation-contraction coupling in the skeletal muscle triad junction. Our results further imply a role of MG29 as a synaptophysin family member in the accurate formation of junctional complexes between the cell surface and intracellular membranes.

The protein similarity information, expression pattern, and map location for the NOV9 protein and nucleic acid disclosed herein suggest that it may have important structural and/or physiological functions characteristic of the Mitsugumin29 family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: Wiskott-Aldrich syndrome, Aldrich syndrome, eczema-thrombocytopenia-immunodeficiency syndrome, thrombocytopenia, night blindness, amyotrophic lateral sclerosis, Batten disease, ceroid lipofuscinosis, Rett syndrome, Pick disease (lobar atrophy), cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection and other diseases, disorders and conditions of the like. The nucleic acids and proteins of the invention are useful in potential diagnostic and therapeutic applications implicated in various diseases and disorders described below and/or other pathologies. For example, the compositions of the present invention will have efficacy for treatment of patients suffering from: diabetes,Von Hippel-Lindau (VHL) syndrome , pancreatitis, obesity, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, transplantation, fertility, endometriosis, Hirschsprung's disease, Crohn's disease, appendicitis and other diseases, disorders and conditions of the like.

The novel nucleic acid encoding the mitsugumin 29 protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV9 protein has multiple hydrophilic regions, each of which can be used as an immunogen. In one embodiment, a contemplated NOV9 epitope is from about amino acids 20 to 25. In another embodiment, a contemplated NOV9 epitope is from about amino acids 30 to 35. In other specific embodiments, contemplated NOV9 epitopes are from about amino acids 60 to 65, 75 to 105, 145 to 155, 170 to 175, 180 to 185 and 240 to 260.

NOV10

The disclosed NOV10 nucleic acid (designated as CuraGen Acc. No. CG56127-01), encodes a novel micromolar calcium-activated neutral protease 1-like protein and includes the 2542 nucleotide sequence (SEQ ID NO: 39) shown in Table 10A. An open reading frame for the mature protein was identified beginning with an ATG codon at nucleotides 260-262 and ending with a TAA codon at nucleotides 2318-2320. Putative untranslated regions downstream from the termination codon and upstream from the initiation codon are underlined in Table 10A, and the start and stop codons are in bold letters.

TABLE 10A
NOV10 Nucleotide Sequence (SEQ ID NO:39)

TTTAACGAAGGTGGAGCCTGCCTTTACCTGGTACACCCATATAAGGAAAA
GCCTGAGGTCAGGAGTAAGCAGACACCAGCACTGCTCTTTCTCCAAGACG
GCCGGCCATGCTCTCCTCCTCTGCCAGTCTCCTCCACCACTCTCTAACCT
GAGAGCCTGTGGAACCTGCCCGTCTCCCCTCCTCCATCAGACACACCTGC
CTAGGAAACAGGTAAGTTGTGCGTGGGGAAAGGACCTCGGAAGTCTTCTA
AGGAGAGTCATGGCGTATTACCAGGAGCCTTCAGTGGAGACCTCCATCAT
CAAGTTCAAAGACCAGGACTTTACCACCTTGCGGGATCACTGCCTGAGCA
TGGGCCGGACGTTTAAGGATGAGACATTCCCTGCAGCAGATTCTTCCATA
GGCCAGAAGCTGCTCCAGGAAAAACGCCTCTCCAATGTGATATGGAAGCG
GCCAGATCTACCAGGGGGTCCTCCTCACTTCATCCTGGATGATATAAGCA
GATTTGACATCCAACAAGGAGGCGCAGGTGACTGCTGGTTCCTGGCAGCA
CTGGGATCCTTGACTCAGAACCCACAGTACAGGCAGAAGATCCTGATGGT
CCAAAGCTTTTCACACCAGTATGCTGGCATTTTCCGTTTCCGGTTCTGGC
AATGTGGCCAGTGGGTGGAAGTGGTGATTGATGACCGCCTACCTGTCCAG
GGAGATAAATGCCTCTTTGTGCGTCCTCGCCACCAAAACCAAGAGTTCTG
GCCCTGCCTGCTGGAGAAGGCCTATGCCAAGCTGCTCGGATCCTATTCCG
ATCTGCACTATGGCTTCCTCGAGGATGCCCTGGTGGACCTCACAGGAGGC
GTGATCACCAACATCCATCTGCACTCTTCCCCTGTGGACCTGGTGAAGGC
AGTGAAGACAGCGACCAAGGCAGGCTCCCTGATAACCTGTGCCACTCCAA
GTGGGGTAAGTCACGATACAGCACAGGCGATGGAGAATGGGCTGGTGAGT
CTCCATGCCTACACTGTGACTGGGGCTGAGCAGGTACAATACCGAAGGGG
CTGGGAAGAAATTATCTCCCTGTGGAACCCCTGGGGCTGGGGCGAGGCCG
AATGGAGAGGGCGCTGGAGTGATGGGTATGGCTTCTGGGACGAAACCTGT
GATCCGCGGAAAAGCCAGCTACATAAGAAACGGGAAGATGGCGAGTTTTG
GTATTTACCCTTCTTATACAATGGTGTTTTAAATCTTTTATTGCCCAAGT
CTTCAATCCCTACCCTTTTCCCTGAACATTTACGAAGGTGGAAAATAGCT
TTGACAGACCCCAGGTGGGCAGGGCCCAGCCCAGGAGGAGCCTGCATTCA
CACACACTCCCATGTCCCAGATAATAAATTCTTTAAAAGAGAGGAAGAAA
AGGAGAAGGAATGCAGGGATGAAACCAATGAGCCAAGCTGTTCGGTTCTG
CTTGCCTTTTTGTTTACGTCTGAGTTCCTAAATCTGCCTTTCTCCCTGTT
TCCAACAGGCTGGCTCACAGGTATGGCTCAGCGTCGTCCTTGCCCTGCGC
CTCTGCTGCTGTCTGCTGGTGGAGTGTTATTTTTTTCCTCGTTCAGAAAC
ACTGTCCAAAGCTCAAATAATAAATTCCGCCGCAACTTCACCATGACTTA
CCATCTGAGCCCTGGGAACTATGTTGTGGTTGCACAGACACGGAGAAAAT
CAGCGGAGTTCTTGCTCCGAATCTTCCATTTCAACCTCAGAATGAAGGTA
GGTATGCAGCAACGTTTGGCTGGCGAGCCTCATTGGCCCCATCCCATCCC
CAAGAGCTTCCGTCTCCTTCTTTACACCTCCCGCTGCCCCCAACCAATGA
AAAGAGAGACACCACACCCCACTGTCAACACTTCAGTCCTTCCTGTCCTT
CTCTCCTCAGGACCTCCAGGGGACATGTTCTCCTTAGATGAGTGCCGCAG
CTTGGTGGCTCTGATGGAAGTATCCTTTGCGGTCATCCCTCCCATGCTCA
TGTTTTCCAGAAGGTTCAGACAAGCCCTGGAGTCCTCCTCACTCACTCGT
TCACCTGTGGCCCCAGACTTCCTCAGAGGGATCTTCATCAGCCGTGAGCT
GCTGCATCTGGTGACCCTCAGGTACAGCGACAGCGTCGGCAGGGTCAGCT
TCCCCAGCCTGGTCTGCTTCCTGATGCGGCTTGAAGCCATGGCAAGTAGT
CAAAACCTTCCCTTCTTTATCCTAGAGACCTTCCGCAACCTCTCTAAGGA
TGGAAAAGGACTCTACCTGACAGAAATGGAGGTGAGGTTTGGGAAAAAGT
ATTTTAAAGTTCATATGTAAACAAATTTACAAGAAAAAAATCAAACAACC
CCATCAAAAAGTGGGCAAAGGATATGAACAGACACTTCTCAAAAGAAGAC
ATTTATGCAGCCAACAGACACATGAAAAAATGCTCACCATCACTGGCCAT
CAGAGAAACGCAAATCAAAACCACAATGAGATACCATCTCACACCAGTTA
GAATAAAATCAATTTCGCCTCTGTTAGAGCCATTGCAACTTC

The nucleic acid sequence of NOV10 maps to chromosome 2 and has 574 of 909 bases (63%) identical to a gb:GENBANK-ID:AF221129|acc:AF221129.1 mRNA from Bos taurus (Bos taurus micromolar calcium-dependent neutral protease large subunit (CAPN1) mRNA, complete cds) (E=1.4e⁻³¹).

The NOV10 polypeptide (SEQ ID NO: 39) is 686 amino acid residues in length and is presented using the one-letter amino acid code in Table 10B. The SignalP, Psort and/or Hydropathy results predict that NOV10 is likely to be localized microbody (peroxisome) with a certainty of 0.7480. In alternative embodiments, a NOV10 polypeptide is located to the plasma membrane with a certainty of 0.7000, the endoplasmic reticulum (membrane) with a certainty of 0.2000, or the mitochondrial inner membrane with a certainty of 0.1000.

TABLE 10B
Encoded NOV10 Protein Sequence (SEQ ID NO:40)

MAYYQEPSVETSIIKFKDQDFTTLRDHCLSMGRTFKDETFPAADSSIGQK
LLQEKRLSNVIWKRPDLPGGPPHFILDDISRFDIQQGGAGDCWFLAALGS
LTQNPQYRQKILMVQSFSHQYAGIFRFRFWQCGQWVEVVIDDRLPVQGDK
CLFVRPRHQNQEFWPCLLEKAYAKLLGSYSDLHYGFLEDALVDLTGGVIT
NIHLHSSPVDLVKAVKTATKAGSLITCATPSGVSHDTAQAMENGLVSLHA
YTVTGAEQVQYRRGWEEIISLWNPWGWGEAEWRGRWSDGYGFWEETCDPR
KSQLHKKREDGEFWYLPFLYNGVLNLLLPKSSIPTLFPEHLRRWKIALTD
PRWAGPSPGGACIHTHSHVPDNKFFKREEEKEKECRDETNEPSCSVLLAF
LFTSEFLNLPFSLFPTGWLTGMAQRRPCPAPLLLSAGGVLFFSSFRNTVQ
SSNNKFRRNFTMTYHLSPGNYVVVAQTRRKSAEFLLRIFHFNLRMKVGMQ
QGLAGEPHWPHPIPKSFRLLLYTSRCPQPMKRETPHPTVNTSVLPVLLSS
GPPGDMFSLDECRSLVALMEVSFAVIPPMLMFSRRFRQALESSSLTRSPV
APDFLRGIFISRELLHLVTLRYSDSVGRVSFPSLVCFLMRLEAMASSQNL
PFFILETFRNLSKDGKGLYLTEMEVRFGKKYFKVHM

The NOV10 amino acid sequence has 194 of 503 amino acid residues (38%) identical to, and 258 of 503 amino acid residues (51%) similar to, the 703 amino acid residue ptnr:SPTREMBL-ACC:Q64698 protein from Rattus norvegicus (Rat) (CALPAIN, LARGE (CATALYTIC) SUBUNIT (EC 3.4.22.17) (CALCIUM-ACTIVATED NEUTRAL PROTEINASE) (CANP) (STOMACH-SPECIFIC CALCIUM-ACTIVATED NEUTRAL PROTEASE LARGE SUBUNIT) (NCL2)) (E=7.2e⁻⁸⁰).

NOV10 is expressed in at least the following tissues: pancreas, colon, skin, lung, breast, uterus, placenta, lymph, leukopheresis, eye, and marrow. This information was derived by determining the tissue sources of the sequences that were included in the invention including but not limited to SeqCalling sources, Public EST sources, Literature sources, and/or RACE sources.

NOV10 also has homology to the amino acid sequences shown in the BLASTP data listed in Table 10C.

TABLE 10C
BLAST results for NOV10

| Gene Index/ | Protein/ | | | | |
|---|---|---|---|---|---|
| Identifier | Organism | Length (aa) | Identity (%) | Positives (%) | Expect |
| gi\|14041821\|dbj\|BAB | stomach-specific | 703 | 174/510 | 236/510 | 7e−66 |
| 55000.1\| (AB061518) | calpain [Mus | | (34%) | (46%) | |
| | musculus] | | | | |
| gi\|9280814\|gb\|AAF63 | calpain [Xenopus | 702 | 204/670 | 302/670 | 8e−66 |
| 194.2\| (AF212199) | laevis] | | (30%) | (44%) | |
| gi\|477319\|pir\|\|A487 | large chain 2, | 703 | 176/510 | 236/510 | 2e−65 |
| 64 calpain (EC | tissue-specific - | | (34%) | (45%) | |
| 3.4.22.17) | rat | | | | |
| gi\|495222\|dbj\|BAA03 | calpain [Rattus | 703 | 175/510 | 235/510 | 2e−64 |
| 369.1\| (D14478) | norvegicus] | | (34%) | (45%) | |
| gi\|2584822\|gb\|AAC04 | calpain Lp82 | 709 | 199/696 | 311/696 | 3e−63 |
| 848.1\| (U96367) | [Rattus | | (28%) | (44%) | |
| | norvegicus] | | | | |

The homology of these sequences is shown graphically in the ClustalW analysis shown in Table 10D.

[image]

[image]

[image]

Tables 10E and 10F list the domain description from DOMAIN analysis results against NOV10. This indicates that the NOV10 sequence has properties similar to those of other proteins known to contain these domains.

TABLE 10E
Domain Analysis of NOV10

gnL|Smart|smart00230, CysPc, Calpain-like thiol protease family, Calpain-like
thiol protease family (peptidase family C2). Calcium activated neutral protease
(large subunit).
Length = 323 residues, 92.0% aligned
Score = 253 bits (645), Expect = 3e−68

| | | | | |
|---|---|---|---|---|
| Query: | 16 | FKDQDFTTLRDHCLSMGRTFKDETFPAADSSIGQKLLQEKRLSNVIWKRPDLPGGPPHFI | 75 | (SEQ ID NO:118) |
| | | \|++\|\|+ \|\| \| \| \| \|\|\| \|\|+ \|\| \| \|+\|\|\|\| \| \| | | |
| Sbjct: | 1 | FENQDYEELRQECLEEGGLFVDPLFPAKPSSLFFSQLQRKF---VVWKRPHEIFEDPPLI | 57 | |
| Query: | 76 | LDDISRFDIQQGGAGDCWFLAALGSLTQNPQYRQKILMV-QSFSEQYAGIFRFRFWQCGQ | 134 | |
| | | + \|\| \|\| \|\| \|\|\|\| \|\|\|\| +\|\| + +++ \| \|\| \|\|\|\|+ \|\|\|\|+ \|+ | | |
| Sbjct: | 58 | VGGASRTDICQGVLGDCWLLAALAALTLREELLARVIPKDQEFSENYAGIYHFRFWRYGK | 117 | |
| Query: | 135 | WVEVVIDDRLPVQGDKCLFVRPRHQNQEFWPCLLEKAYAKLLGSYSDLHYGFLEDALVDL | 194 | |
| | | \|\|+\|\|\|\|\|\|\|\| \|\|+ +\| \|\|\| \|\|\|\|\|\|\|\|\| \| \| \| \| +\|\| \|\| | | |
| Sbjct: | 118 | WVDVVIDDRLPTYNGDLLFMHSNSRN-EFWSALLEKAYAKLRGCYEALKGGSTTEALEDL | 176 | |
| Query: | 195 | TGGVITNIHLHSSPVD---LVKAVKTATKAGSLITCATPSGVSHDTAQAMENGLVSLHAY | 251 | |
| | | \|\|\|\| +\| \| \| \| \| +\| \| + \|\|\|+ \|+ +\| + + + \|\|\|\| \|\|\| | | |
| Sbjct: | 177 | TGGVAESIELKKISKDPDELFKDLKKAFERGSLMGCSIGAGTAVEEEEQKRNGLVKGHAY | 236 | |
| Query: | 252 | TVTGAEQVQYRRGWEEIISLWNPWGWGEAEWRGRWSDGYGFWEE-TCDPRKSQLHKKRED | 310 | |
| | | +\|\| +\| \|\| ++++ \| \|\| \|\|\|+\|\| \| \|\|\| \| + + +\|+ +\| | | |
| Sbjct: | 237 | SVTDVREVDGRRR-QKLLRLRNP--WGESEWNGPWSDDSPEWRSVSAEEKKNLGLTMDDD | 293 | |
| Query: | 311 | GEFW 314 | | |
| | | \|\|\|\| | | |
| Sbjct: | 294 | GEFW 297 | | |

TABLE 10F
Domain Analysis of NOV10

gnL|Pfam|pfam00648, Peptidase_C2, Calpain family cysteine protease (SEQ ID NO:119)
Length = 298 sidues, 96.3 aligned
Score = 221 bits (564), Expect = 8e−59

| | | | |
|---|---|---|---|
| Query: | 35 | FKDETFPAADSSIGQELLQEKRLSNVIWKRPDLPGGPPHFILDDISRFDIQQGGAGDCWF | 94 |
| | | \| \| +\|\|\|\| \|+\| \| \| + + \|\|\|\| \| \|\|+ +\| \|\| \|\| \|\|\|\| | |
| Sbjct: | 2 | FVDPSFPAAPKSLGYKPLGPRG---IEWKRPHEINENPQFIVGGATRTDICQGALGDCWL | 58 |
| Query: | 95 | LAALGSLTQNPQ-YRQKILMVQSFSHQYAGIFRFRFWQCGQWVEVVIDDRLPVQGDKCLF | 153 |
| | | \|\|\|\| \|\|\| \| + + \|\|\| \|\|\|\|\| \|\|\|\|\| \|+\|\|+\|\|+\|\| \|\| + \| \|\| | |
| Sbjct: | 59 | LAALASLTLNEPLLLRVVPHDQSFQENYAGIFHFRFWQFGEWVDVVVDDLLPTKDGKLLF | 118 |
| Query: | 154 | VRPRHQNQEFWPCLLEKAYAKLLGSYSDLHYGFLEDALVDLTGGVITNIHLHSSP---VD | 210 |
| | | \| +\| \|\|\| \|\|\|\|\|\|\|\|\| \| \| \| \| +\|\| \|\|\|\|\|\| + \| +\| ++ | |
| Sbjct: | 119 | VHSAERN-EFWSALLEKAYAKLNGCYEALSGGSTTEALEDLTGGVCESYELKLAPSSMLN | 177 |
| Query: | 211 | LVKAVKTATKAGSLITCATPSGVSHDTAQAMENGLVSLHAYTVTGAEQVQYRRGWEEIIS | 270 |
| | | \| +\| + \|\|\|+ \|+ \| \| \|\|\| \|\|\|+\|\|\| ++\| \|\| ++\| | |
| Sbjct: | 178 | LGNIIKKMLERGSLLGCSIDITSPVDMEARMAKGLVKCHAYSVTGVKEVNYRGEGVKLIR | 237 |
| Query: | 271 | LWNPWGWGEAEWRGRWSDGYQFWEETCDPRKSQLHKKREDGEFW--YLPFLYN 321 | |
| | | \| \|\| \|\|+ \|\| \| \|\|\| \| \|++\| \| \|\|\|\|\|\| + \|\| + | |
| Sbjct: | 238 | LRNP--WGQVEWTGDWSDSSPDWNIVDPDEKARLQLKFEDGEFWMSFEDFLRH 288 | |

The predicted sequence described here belongs to the calpain protease family. The calpains, or calcium-activated neutral proteases, are nonlysosomal intracellular cysteine proteases (Richard, et al.). Calpain is an intracellular protease involved in many important cellular functions that are regulated by calcium. The mammalian calpains include 2 ubiquitous proteins, CAPN1 and CAPN2, as well as 2 stomach-specific proteins, and CAPN3, which is muscle-specific. The ubiquitous enzymes consist of heterodimers with distinct large subunits associated with a common small subunit, all of which are encoded by different genes. The association of tissue-specific large subunits with a small subunit has not yet been demonstrated. The large subunits of calpains can be subdivided into 4 domains; domains I and III, whose functions remain unknown, show no homology with known proteins. The former, however, may be important for the regulation of the proteolytic activity. Domain II shows similarity with other cysteine proteases, which share histidine, cysteine, and asparagine residues at their active sites. Domain IV comprises 4 EF-hand structures that are potential calcium-binding sites. In addition, 3 unique regions with no known homology are present in the muscle-specific CAPN protein, namely NS, IS1, and IS2, the latter containing a nuclear translocation signal. These regions may be important for the muscle-specific function of CAPN3 (Richard, et al.).

It was previously shown that defects in the human calpain 3 gene are responsible for limb girdle muscular dystrophy type 2A (LGMD2A), an inherited disease affecting predominantly the proximal limb muscles. To better understand the function of calpain 3 and the pathophysiological mechanisms of LGMD2A and also to develop an adequate model for therapy research, we generated capn3-deficient mice by gene targeting. capn3-deficient mice are fully fertile and viable. Allele transmission in intercross progeny demonstrated a statistically significant departure from Mendel's law. capn3 -deficient mice show a mild progressive muscular dystrophy that affects a specific group of muscles. The age of appearance of myopathic features varies with the genetic background, suggesting the involvement of modifier genes. Affected muscles manifest a similar apoptosis-associated perturbation of the IkappaBalpha/nuclear factor kappaB pathway as seen in LGMD2A patients. In addition, Evans blue staining of muscle fibers reveals that the pathological process due to calpain 3 deficiency is associated with membrane alterations (Richard, et al.).

Recently, calpain was suggested to be involved in the progression of alpha-fodrin proteolysis and tissue destruction in the development of Sjogren syndrome (SS) (Hayashi et al.). SS is an autoimmune disease characterized by diffuse lymphoid cell infiltrates in the salivary and lacrimal glands, resulting in symptoms of dry mouth and eyes due to insufficient secretion. Although it has been assumed that a combination of immunologic, genetic and environmental factors may play a key role in the development of autoimmune lesions in the salivary and lacrimal glands, little is known about the disease pathogenesis of SS in humans. The 120 kDa alpha-fodrin as an important autoantigen in the development of SS in both an animal model and SS patients, but the mechanism of alpha-fodrin cleavage leading to tissue destruction in SS remains unclear. Tissue-infiltrating CD4+ T cells purified from the salivary glands of a mouse model for SS bear a large proportion of Fas ligand and the salivary gland duct cells possess apoptotic receptor Fas. Anti-Fas antibody-induced apoptotic salivary gland cells result in specific alpha-fodrin cleavage to the 120 kDa fragment in vitro. Preincubation with a combination of calpain and caspase inhibitor peptides could be responsible for inhibition of the 120 kDa alpha-fodrin cleavage. Thus, an increase in apoptotic protease activities including calpain and caspases may be involved in the progression of alpha-fodrin proteolysis and tissue destruction in the development of SS (Hayashi et al.).

It is anticipated that the novel sequence described here will have useful properties and functions similar to calpain proteases because of the presence of the Calpain-type cystein-protease (C2 family) domain and the homology to calpain III.

The protein similarity information, expression pattern, and map location for the NOV10 protein and nucleic acid disclosed herein suggest that it may have important structural and/or physiological functions characteristic of the cysteine protease family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: diabetes, Von Hippel-Lindau (VHL) syndrome, pancreatitis, obesity, hypercalceimia, ulcers, endometriosis, fertility, hemophilia, hypercoagulation, idiopathic thrombocytopenic purpura, autoimmume disease, allergies, immunodeficiencies, transplantation, graft versus host disease, psoriasis, actinic keratosis, tuberous sclerosis, acne, hair growth/loss, allopecia, pigmentation disorders, endocrine disorders, hemophilia, lymphaedema, and other diseases, disorders and conditions of the like.

The novel nucleic acid encoding micromolar calcium-activated neutral protease-1 protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV10 protein has multiple hydrophilic regions, each of which can be used as an immunogen. In one embodiment, a contemplated NOV10 epitope is from about amino acids 5 to 90. In another embodiment, a contemplated NOV10 epitope is from about amino acids 105 to 110. In other specific embodiments, contemplated NOV10 epitopes are from about amino acids 170 to 180, 230 to 310, 370 to 400, 420 to 430, 450 to 455, 460 to 465, 480 to 485, 510 to 515, 570 to 580 and 680 to 690.

NOV11

A disclosed NOV11 nucleic acid (designated CuraGen Acc. No. CG56179-01) encodes a novel P2X2C-like protein and includes the 1422 nucleotide sequence (SEQ ID NO: 41) which is shown in Table 11A. An open reading frame was identified beginning with an ATG initiation codon at nucleotides 1-3 and ending with a TGA codon at nucleotides 1420-1422. The start and stop codons are in bold letters in Table 11A.

TABLE 11A
NOV11 Nucleotide Sequence (SEQ ID NO:41)

ATGGCCGCCGCCCAGCCCAGTACCCCGCCGGGGCGACCGCCCGGCGCCTG
GCCCGGGGCTGCTGGTCCGCCCTCTGGGACTACGAGACGCCCAAGGTGAT
CGTGGTGAGGAACCGGCGCCTGGGGGTCCTGTACCGCGCCGTGCAGCTGC
TCATCCTGCTCTACTTCGTGTGGTACGTATTCATCGTGCAGAAAAGCTAC
CAGGAGAGCGAGACGGGCCCCCAGAGCTCCATCATCACCAAGGTCAAGGG
GATCACCACGTCCGAGCACAAAGTGTGGGACGTGGAGGAGTACGTGAAGC
CCCCCGAGAGCATAAGGCTCCACAACGCCACCTGCCTCTCCGACGCCGAC
TGCGTGGCTGCGGAGCTGGACATCCTGGGAAACGGCCTGAGGACCGGGCG
CTGTGTGCCCTATTACCAGGGGCCCTCCAAGACCTGCGAGGTGTTCGGCT
GGTGCCCGGTGGAAGATGGGGCCTCTGTCAGCCAATTTCTGGGTACGATG
GCCCCAAATTTCACCATCCTCATCAAGAACAGCATCCACTACCCCAAATT
CCACTTCTCCAAGGGCAACATCGCCGACCGCACAGACGGGTACCTGAAGC
GCTGCACGTTCCACGAGGCCTCCGACCTCTACTGCCCCATCTTCAAGCTG
GGCTTTATCGTGGAGAAGGCTGGGGAGAGCTTCACAGAGCTCGCACACAA
GGGTGGTGTCATCGGGGTCATTATCAACTGGGACTGTGACCTGGACCTGC
CTGCATCGGAGTGCAACCCCAAGTACTCCTTCCGGAGGCTTGACCCCAAG
CACGTGCCTGCCTCGTCAGGCTACAACTTCAGGTTTGCCAAATACTACAA
GATCAATGGCACCACCACCCGCACGCTCATCAAGGCCTACGGGATCCGCA
TTGACGTCATTGTGCATCGACAGGCCGGGAAGTTCAGCCTGATTCCCACC
ATTATTAATCTGGCCACAGCTCTGACTTCCGTCGGGGTGGTAAGGAACCC
TCTCTGGGGTCCCAGCGGGTGCGGGGGGTCCACCAGGCCCTTACACACCG
GTCTCTGCTGGCCCCAGGGCTCCTTCCTGTGCGACTGGATCTTGCTAACA
TTCATGAACAAAAACAAGGTCTACAGCCATAAGAAATTTGACAAGGTGTG
TACGCCGAGCCACCCCTCACGTAGCTGGCCTGTGACCCTTGCCCGTGTAT
TGGGCCAGGCCCCTCCCGAACCCGGCCACCGCTCCGAGGACCAGCACCCC
AGCCCTCCATCAGGCCAGGAGGGCCAACAAGGGGCAGAGTGTGGCCCAGC
CTTCCCGCCCCTGCGGCCTTGCCCCATCTCTGCCCCTTCTGAGCAGATGG
TGGACACTCCTGCCTCCGAGCCTGCCCAAGCCTCCACACCCACAGACCCC
AAAGGTTTGGCTCAACTCTGA

The nucleic acid sequence of NOV11 maps to chromosome 1 and has 990 of 991 bases (99%) identical to a gb:GENBANK-ID:AF190824|acc:AF190824.1 mRNA from Homo sapiens (Homo sapiens P2X2C receptor (P2X2) mRNA, complete cds) (E=3.6e⁻²⁹⁵).

A NOV11 polypeptide (SEQ ID NO: 42) is 473 amino acid residues and is presented using the one letter code in Table 11B. The SignalP, Psort and/or Hydropathy results predict that NOV11 has a signal peptide and is likely to be localized to the mitochondrial inner membrane with a certainty of 0.6577. In alternative embodiments, a NOV11 polypeptide is located to the plasma membrane with a certainty of 0.6500, the microbody (peroxisome) with a certainty of 0.3556, or the Golgi body with a certainty of 0.3000. The SignalP predicts a likely cleavage site for a NOV11 peptide between amino acid positions 68 and 69, i.e. at the dash in the sequence SYQ-ES.

TABLE 11B
NOV11 protein sequence

(SEQ ID NO:42)

MAAAQPKYPAGATARRLARGCWSALWDYETPKVIVVRNRRLGVLYRAVQLLILLYFVWYVFIVQKSYQESETGP
ESSIITKVKGITTSEHKVWDVEEYVKPPESIRVHNATCLSDADCVAGELDMLGNGLRTGRCVPYYQGPSKTCEV
FGWCPVEDGASVSQFLGTMAPNFTILIKNSIHYPKFHFSKGNIADRTDGYLKRCTFHEASDLYCPIFKLGFIVE
KAGESFTELAHKGGVIGVIINWDCDLDLPASECNPKYSFRRLDPKHVPASSGYNFRFAKYYKINGTTTRTLIKA
YGIRIDVIVHGQAGKFSLIPTIINLATALTSVGVVRNPLWGPSGCGGSTRPLHTGLCWPQGSFLCDWILLTFMN
KNKVYSHKKFDKVCTPSHPSGSWPVTLARVLGQAPPEPGHRSEDQHPSPPSGQEGQQGAECGPAFPPLRPCPIS
APSEQMVDTPASEPAQASTPTDPKGLAQL

The NOV11 amino acid sequence have 330 of 330 amino acid residues (100%) identical to, and 330 of 330 amino acid residues (100%) similar to, the 447 amino acid residue ptnr:SPTREMBL-ACC:Q9UHD6 protein from Homo sapiens (Human) (P2X2C RECEPTOR) (E=8.7e⁻²⁴⁸).

NOV11 is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. This information was derived by determining the tissue sources of the sequences that were included in the invention including but not limited to SeqCalling sources, Public EST sources, Literature sources, and/or RACE sources.

Possible small nucleotide polymorphisms (SNPs) found for NOV11 are listed in Tables 11C and 11D.

TABLE 11C
SNPs

| Consensus | | Base | |
|---|---|---|---|
| Position | Depth | Change | PAF |
| 273 | 23 | G > A | 0.304 |

TABLE 11D
SNPs

| | Nucleotide | Base | Amino Acid | Base |
|---|---|---|---|---|
| Variant | Position | Change | Position | Change |
| 13374572 | 1121 | T > C | 374 | Val > Ala |

NOV11 also has homology to the amino acid sequences shown in the BLASTP data listed in Table 11E.

TABLE 11E
BLAST results for NOV11

| Gene Index/ | | Length | Identity | Positives | |
|---|---|---|---|---|---|
| Identifier | Protein/Organism | (aa) | (%) | (%) | Expect |
| gi\|6606328\|gb\|AAF1 | P2X2D receptor | 497 | 473/497 | 473/497 | 0.0 |
| 9173.1\|AF190825_1 | [Homo sapiens] | | (95%) | (95%) | |
| (AF190825) | | | | | |
| gi\|7706629\|ref\| | P2X2C receptor; | 447 | 447/473 | 447/473 | 0.0 |
| NP_057402.1\| | P2X Receptor, | | (94%) | (94%) | |
| (NM_016318) | Subunit 2 [Homo | | | | |
| | sapiens] | | | | |
| gi\|12643353\|sp\| | P2X PURINOCEPTOR | 471 | 447/497 | 447/497 | 0.0 |
| Q9UBL9\|P2X2_HUMAN | 2 (ATP RECEPTOR) | | (89%) | (89%) | |
| | (P2X2) | | | | |
| | (PURINERGIC | | | | |
| | RECEPTOR) | | | | |
| gi\|5381337\|gb\| | P2X2A receptor | 459 | 433/483 | 433/483 | 0.0 |
| AAD42947.1\|AF10938 | [Homo sapiens] | | (89%) | (89%) | |
| 7_1 (AF109387) | | | | | |
| gi\|6606324\|gb\|AAF1 | P2X2B receptor | 404 | 360/426 | 362/426 | 0.0 |
| 9171.1\|AF190823_1 | [Homo sapiens] | | (84%) | (84%) | |
| (AF190823) | | | | | |

The homology of these sequences is shown graphically in the ClustalW analysis shown in Table 11F.

[image]

[image]

[image]

Table 11G lists the domain description from DOMAIN analysis results against NOV11. This indicates that the NOV11 sequence has properties similar to those of other proteins known to contain these domains.

TABLE 11G
Domain Analysis of NOV11

gnl|Pfam|pfam00864, P2X receptor, ATP P2X receptor (SEQ ID NO:125)
Length = 377 residues, 96.6% aligned
Score = 509 bits (1310), Expect = 2e−145

| | | | |
|---|---|---|---|
| Query: | 26 | WDYETPKVIVVRNRRLGVLYRAVQLLILLYFVWYVFIVQKSYQESETGPESSIITKVKGI | 85 |
| | | +\|\|+\|\|\| +\|\|\|\|+++\|+\| \| \|\|\|\|\|\|+\| \| +\|\|+++\| \|\|+\|+\| +\|\|+\|\|\|\|\|\|+ | |
| Sbjct: | 1 | FDYKTPKYVVVRNKKVGLLNRLVQLLILVYVVGWVFLIEKGYQDSDTSLQSSVITKVKGV | 60 |
| Query: | 86 | TTSE-----HKVWDVEEYVKP------------------------PESIRVHNATCLSDA | 116 |
| | | + ++\|\|\|\| +\|\| \| \|\| \| + \|\| \|\|+ | |
| Sbjct: | 61 | AVTNTSELGNRVWDVADYVIPPQGENVFFVVTNFIVTPNQTQGTCPEHPEVPDGTCKSDS | 120 |
| Query: | 117 | DCVAGELDMLGNGLRTGRCVPYYQGPSKTCEVFGWCPVEDGASVSQFLGTMAPNFTILIK | 176 |
| | | \|\| \|\|\| \|\|\|++\|\|\|\|\| + +\|\|\|+\| \|\|\|\|\| + \| \| \|\|\|\| \|\| | |
| Sbjct: | 121 | DCTAGEAGTHGNGIKTGRCVAFNGSVRRTCEIFAWCPVEVDTVPNPPLLKEAENFTIFIK | 180 |
| Query: | 177 | NSIHYPKFHFSKGNIAD-RTDGYLKRCTFHEASDLYCPIFKLGFIVEKAGESFTELAHKG | 235 |
| | | \|\|\| +\|\|\|+\|\|\|\|\|+ + +\|\| \|\|\| \| \|\| +\| \|\|\|\|\|+\|\| +\|\|\|\|\|+ \| +\|\| \|\| | |
| Sbjct: | 181 | NSIRFPKFNFSKGNLLENKTDTYLKHCRFHPTNDPYCPIFRLGDVVEKAGQDFQDLALKG | 240 |
| Query: | 236 | GVIGVIINWDCDLDLPASECNPKYSFRRLDPKHV-PASSGYNFRFAKYYKI-NGTTTRTL | 293 |
| | | \|\|\|\|+\|\|\|\|\|\|\|\|\| \|\|\|\|\|\| \|\|\|\|\|\|\| \| \| \|\|\|\|\|\|\|\|\|\|+ \|\| \|\|\| | |
| Sbjct: | 241 | GVIGIIINWDCDLDKAASECNPHYSFRRLDNKKEKSVSPGYNFRFAKYYRDNNGVEYRTL | 300 |
| Query: | 294 | IKAYGIRIDVIVIGQAGKFSLIPTIINLATALTSVGVVRNPLWGPSGCGGSTRPLHTGLC | 353 |
| | | +\|\|\|\|\|\| \|\|+\|+\|+\|\|\|\| +\|\|\|\|\|\|+ + \| \|+\|\| | |
| Sbjct: | 301 | LKAYGIRFDVLVNGKAGKFDIIPTIINIGSGLASLGV----------------------- | 337 |
| Query: | 354 | WPQGSFLCDWILLTFMNKNKVYSHKKFDKV | 383 |
| | | \|+\|\|\|\| \|\|\| \|+ \| \| \|\|\|++\| | |
| Sbjct: | 338 | ---GTFLCDLILLYFLKKRHFYRDKKFEEV | 364 |

P2X receptors are membrane ion channels gated by extracellular adenosine 5′-triphosphate (ATP); nucleotides also activate a family of seven transmembrane G protein-coupled receptors (P2Y). P2X receptors are widely expressed on mammalian cells, where they can be broadly differentiated into three groups. The first group is almost equally well activated by ATP and its analog alpha beta methyleneATP (alpha beta meATP), whereas a second group is not activated by alpha beta meATP. A third-group type of receptor (termed P2Z) is distinguished by the fact that the channel opening is followed by cell permeabilization and lysis if the agonist application is continued for more than a few seconds. Seven cDNAs have been cloned that encode P2X receptor subunits. When expressed individually in heterologous systems, P2X1 and P2X3 subunits form channels activated by ATP or alpha beta meATP; whereas P2X2, P2X4, and P2X5 form channels activated by ATP but not alpha beta meATP. P2X6 receptors do not express readily, and P2X7 receptors correspond closely in their properties to P2Z. Further phenotypes can be produced when two subunits are coexpressed, indicating hetero-multimerization. Electrophysiological experiments on dissociated smooth muscle and neurons have revealed three distinct phenotypes of P2X receptor: (1) a rapidly desensitizing, beta-methylene ATP-sensitive response typical of most smooth muscle; (2) a non-desensitizing, alpha,beta-methylene ATP-insensitive response characteristic of PC 12 phaeochromocytoma cells and rat superior cervical ganglion neurons; and (3) a non-desensitizing, alpha, beta-methylene ATP-sensitive response observed in sensory neurons.

All of these purinoceptors share a similar cationic and high Ca2+ permeability and sensitivity to blockade by suramin, Cibacron blue, oxidized ATP, pyridoxal-5-phosphate and pyridoxalphosphate-6-azophenyl-2′,4′-disulfonic acid. Heterologous expression of two forms of cloned P2X receptors (from rat vas deferens and PC 12 cells) reveals that each cloned receptor can reconstitute native responses with remarkable fidelity. Such results suggest that homo-oligomeric channels may be formed from single subunits of the P2X receptor in smooth muscle, PC 12 cells and some neurons. The third phenotype observed in native cells might result from co-assembly of subunits of the cloned receptors. However, co-expression studies show that these two forms of the P2X receptor do not heteropolymerize. Therefore, the non-desensitizing, alpha, beta-methylene ATP-sensitive response observed in sensory neurons may result from a distinct P2X receptor or from heteropolymerization of more than one distinct P2X purinoceptor.

There are seven P2X receptor cDNAs currently known. Six homomeric (P2X1, P2X2, P2X3, P2X4, P2X5, P2X7) and three heteromeric (P2X2/P2X3, P2X4/P2X6, P2X1/P2X5) P2X receptor channels have been characterized in heterologous expression systems. Homomeric P2X 1 and P2X3 receptors are readily distinguishable by their rapid desensitization, the agonist action of alpha beta methyleneATP, and the block by 2′,3′-O-(2,4,6-trinitrophenyl)-ATP. P2X2 receptors are unique among homomeric forms in their potentiation by low pH. Homomeric P2X4 receptors are much less sensitive to antagonism by suramin and pyridoxal 5-phosphate-6-azo-2′,4′-disulfonic acid. Homomeric P2X7 receptors are the only form in which 2′,3′-O-(4-benzoylbenzoyl)-ATP is more potent than ATP. The heteromeric P2X2/P2X3 receptor resembles P2X2 in slow desensitization kinetics and potentiation by low pH and is similar to P2X3 with respect to agonism by alpha beta methyleneATP and block by 2′,3′-O-(2,4,6-trinitrophenyl)-ATP. Seven subtypes of P2X receptor family of ligand-gated ion channels (responsive to ATP) have been identified, which form homo-multimeric or hetero-multimeric pores. P2X3 receptors are selectively expressed predominantly on small-diameter nociceptive sensory neurones in the dorsal root, trigeminal and nodose ganglia, particularly the non-peptidergic subpopulations labelled with the lectin IB4. P2X2/3 labelling is also present in inner lamina II of the spinal cord and in sensory nerve projections to skin and viscera, but few receptors are present in skeletal muscle. P2X3 receptors are down-regulated after peripheral nerve injury and their expression can be regulated by glial cell-derived neurotrophic factor. P2X receptor activation of sensory neurones has been demonstrated in in vivo pain models, including the rat hindpaw and knee-joint preparations, as well as in inflammatory models. P2X4 and/or P2X6 receptors in the CNS also seem to be involved in pain pathways. Non-nociceptive P2 receptors on sensory nerves are present in muscle and on sensory endings in the heart and lung that initiate reflex activity involving vagal afferent and efferent nerve fibres.

The protein similarity information, expression pattern, and map location for the NOV11 protein and nucleic acid disclosed herein suggest that it may have important structural and/or physiological functions characteristic of the ATP P2X receptor family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention may have efficacy for treatment of patients suffering from pain, since P2X receptor activation of sensory neurones has been demonstrated in in vivo pain models, including the rat hindpaw and knee-joint preparations, as well as in inflammatory models. P2X4 and/or P2X6 receptors in the CNS also seem to be involved in pain pathways. Non-nociceptive P2 receptors on sensory nerves are present in muscle and on sensory endings in the heart and lung that initiate reflex activity involving vagal afferent and efferent nerve fibres (Br J Anaesth 2000 Apr;84(4):476-88). The compositions of the present invention may also have efficacy for treatment of patients suffering from diabetes, obesity, syndrome X, and other diseases, disorders and conditions of the like.

The novel nucleic acid encoding the P2X2C-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV11 protein has multiple hydrophilic regions, each of which can be used as an immunogen. In one embodiment, a contemplated NOV11 epitope is from about amino acids 5 to 10. In another embodiment, a contemplated NOV11 epitope is from about amino acids 20 to 25. In other specific embodiments, contemplated NOV11 epitopes are from about amino acids 40 to 50, 70 to 80, 95 to 105, 140 to 148, 195 to 215, 250 to 300, 340 to 360, 370 to 380, 410 to 430 and 455 to 465.

NOV12

A disclosed NOV12 nucleic acid (designated CuraGen Acc. No. CG56132-01) encodes a novel DIABLO-like protein and includes 1823 nucleotides (SEQ ID NO: 43) which is shown in Table 12A. An open reading frame was identified beginning with an ATG initiation codon at nucleotides 31-33 and ending with a TGA codon at nucleotides 1606-1608. Putative untranslated regions upstream from the initiation codon and downstream from the termination codon are underlined in Table 12A, and the start and stop codons are in bold letters.

TABLE 12A
NOV12 Nucleotide Sequence

(SEQ ID NO:43)

AACTGTCTATGCTTTTTTATTTTTCTGTAGATGGACCACACATCCCCGACCTACATGCTTGCTAACTTAA
CCCACTTGCATTCTGAACAACTTCTGCAGGGCTTGAATCTTCTTCGCCAACATCACGAACTCTGTGACAT
CATTCTTCGAGTAGGTGATGTTAAAATTCATGCTCACAAAGTGGTACTTGCCAGCGTCAGCCCGTATTTC
AAAGCTATGTTCACTGGAAACCTTTCTGAAAAAGAGAACAGTGAGGTTGAGTTTCAATGCATTGATGAAA
CTGCTCTCCAGGCCATTGTGGAGTATGCCTATACAGGGACTGTTTTTATTTCTCAGGACACAGTTGAATC
TCTCCTGCCAGCAGCAAACCTACTCCAGATAAAACTTGTCCTGAAAGAATGTTGTGCATTTCTTGAAAGC
CAACTTGATCCTGGTAATTGTATTGGAATTTCTCGTTTTGCAGAAACATATGGTTGCCGTGACCTTTATT
TGGCAGCCACTAAATACATATGCCAGAATTTTGAAGCTGTTTGCCAGACTGAAGAGTTTTTTGAGCTTAC
ACATGCTGACTTGGATGAAATTGTTTCCAATGACTGTTTGAATGTAGCTACCGAAGAGACTGTTTTTTAT
GCATTAGAGTCTTGGATCAAGTATGATGTACAAGAACGCCAGAAATACTTAGCACAGTTACTAAACAGTG
TACGATTACCATTGTTGAGTGTTAAGTTTCTCACTAGACTATATGAAGCAAATCATCTTATTCGTGATGA
TCGCACTTGTAAACATCTTTTGAATGAAGCCCTAAAGTACCACTTTATGCCTGAACATAGACTCTCTCAT
CAGACAGTCTTGATGACACGACCTCGCTGTGCTCCCAAAGTACTTTGTGCAGTAGGAGGGAAATCTGGAC
TCTTTGCCTGTTTGGATAGGGTCACTATCAGAAAACATGAAAATTCAGTGGAATGCTGGAATCCTGATAC
AAATACTTGGACTTCTCTAGAGAGAATGAATGAAAGCCGAAGTACTCTTGGAGTAGTAGTACTTGCAGGA
GAACTTTATGCCTTAGGTGGTTATGATGGACAATCTTATTTACAATCTGTAGAGAAGTACATTCCCAAAA
TAAGAAAATGGCAACCTGTGGCACCAATGACGACAACAAGAAGTTGTTTTGCTGCAGCGGTATTGGATGG
AATGATATATGCCATTGGTGGGTATGGTCCTGCCCACATGAACAGTGTGGAGCGTTATGATCCAAGTAAG
GACTCCTGGGAGATGGTTGCATCCATGGCAGATAAAAGGATTCACTTTGGCGTGGGTGTCATGCTAGGCT
TTATTTTTGTGGTGGGTGGACATAATGGAGTCTCACATTTGTCCAGCATTGAAAGATACGATCCTCATCA
AAATCAGTGGACTGTGTGTAGACCAATGAAAGAACCTAGAACAGGAGTTGGTGCTGCTGTAATCGATAAC
TACCTTTATGTCGTCGGTGGTCACTCAGGGTCTTCCTATCTGAATACAGTGCAGAAATATGATCCTATCT
CAGATACGTGGCTGGATTCAGCTGGCATGATATACTGTCGCTGCAACTTTGGGTTAACTGCACTTTGACA
AATGTGAACTCTCGGAAATAGTATGGTGGTGAAACTTGTACTGCATGAACATCCGGATGGCCCAGTTTTC
TGAAACCCACAAGCTGCATTGCTTTCTTTTTAACTTGAAGTAGCATGAAGGCTCAAAAGTTTTGTTGGGT
ACTTTTAATTGAGAAGTAGTTTTGGTTGCTCTTGATTACACAGTAAATCAATAATCAAAAAAAAAAAAAA
AAA

The nucleic acid sequence of NOV12 invention has 909 of 918 bases (99%) identical to a gb:GENBANK-ID:AK000088|acc:AK000088.1 mRNA from Homo sapiens (Homo sapiens cDNA FLJ20081 fis, clone COL03242) (E=9.3e⁻¹⁹⁸).

A NOV12 polypeptide (SEQ ID NO: 43) is 525 amino acid residues and is presented using the one letter code in Table 12B. The SignalP, Psort and/or Hydropathy results predict that NOV12 is likely to be localized to the endoplasmic reticulum (membrane) with a certainty of 0.8500. In alternative embodiments, a NOV12 polypeptide is located to the plasma membrane with a certainty of 0.4400, the microbody (peroxisome) with a certainty of 0.3084, or the mitochondrial inner membrane with a certainty of 0.1000.

TABLE 12B
NOV12 protein sequence

(SEQ ID NO:44)

MDHTSPTYMLANLTHLHSEQLLQGLNLLRQHHELCDIILRVGDVKIHAHKVVLASVSPYFKAMFTGNLSEKENS
EVEFQCIDETALQAIVEYAYTGTVFISQDTVESLLPAANLLQIKLVLKECCAFLESQLDPGNCIGISRFAETYG
CRDLYLAATKYICQNFEAVCQTEEFFELTHADLDEIVSNDCLNVATEETVFYALESWIKYDVQERQKYLAQLLN
SVRLPLLSVKFLTRLYEANHLIRDDRTCKHLLNEALKYHFMPEHRLSHQTVLMTRPRCAPKVLCAVGGKSGLFA
CLDRVTIRKHENSVECWNPDTNTWTSLERMNESRSTLGVVVLAGELYALGGYDGQSYLQSVEKYIPKIRKWQPV
APMTTTRSCFAAAVLDGMIYAIGGYGPAHMNSVERYDPSKDSWEMVASMADKRIHFGVGVMLGFIFVVGGHNGV
SHLSSIERYDPHQNQWTVCRPMKEPRTGVGAAVIDNYLYVVGGHSGSSYLNTVQKYDPISDTWLDSAGMIYCRC
NFGLTAL

The NOV12 amino acid sequence have 225 of 521 amino acid residues (43%) identical to, and 324 of 521 amino acid residues (62%) similar to, the 623 amino acid residue ptnr:SPTREMBL-ACC:Q9NGX7 protein from Drosophila melanogaster (Fruit fly) (DIABLO) (E=2.0e⁻¹⁰⁹).

The NOV12 in this invention is expressed in at least the following tissues: Foreskin, hypothalamus, kidney, prostate, retina, tonsils, breast, whole organism. This information was derived by determining the tissue sources of the sequences that were included in the invention including but not limited to SeqCalling sources, Public EST sources, Literature sources, and/or RACE sources.

NOV12 also has homology to the amino acid sequences shown in the BLASTP data listed in Table 12C.

TABLE 12C
BLAST results for NOV12

| Gene Index/ | Protein/ | Length | Identity | Positives | |
|---|---|---|---|---|---|
| Identifier | Organism | (aa) | (%) | (%) | Expect |
| gi\|12832769\|dbj\|BAB | putative [Mus | 571 | 511/571 | 520/571 | 0.0 |
| 22250.1\| (AK002637) | musculus] | | (89%) | (90%) | |
| gi\|11434452\|ref\| | hypothetical | 300 | 300/300 | 300/300 | e−175 |
| XP_007355.1\| | protein | | (100%) | (100%) | |
| (XM_007355) | FLJ20081 [Homo | | | | |
| | sapiens] | | | | |
| gi\|8923090\|ref\| | hypothetical | 300 | 298/300 | 298/300 | e−174 |
| NP_060128.1\| | protein | | (99%) | (99%) | |
| (NM_017658) | FLJ20081 [Homo | | | | |
| | sapiens] | | | | |
| gi\|12850547\|dbj\|BAB | putative [Mus | 249 | 243/249 | 247/249 | e−140 |
| 28765.1\| (AK013278) | musculus] | | (97%) | (98%) | |
| gi\|12314036\|emb\|CAC | (A Kelch | 601 | 221/518 | 324/518 | e−120 |
| 10469.1\| (AL109921) | motif- | | (42%) | (61%) | |
| dJ383J4.1 | containing | | | | |
| | protein) [Homo | | | | |
| | sapiens] | | | | |

The homology of these sequences is shown graphically in the ClustalW analysis shown in Table 12D.

[image]

[image]

[image]

Tables 12E, 12F and 12G list the domain description from DOMAIN analysis results against NOV12. This indicates that the NOV12 sequence has properties similar to those of other proteins known to contain these domains.

TABLE 12E
Domain Analysis of NOV12

gnl|Pfam|pfam00651, BTB, BTB/POZ domain. (SEQ ID NO:131)
Length = 114 residues, 100% aligned
Score = 122 bits (306), Expect = 5e−29

| | | | |
|---|---|---|---|
| Query: | 19 | EQLLQGLNLLRQHHELCDIILRVGDVKIHAHKVVLASVSPYFKAMFTGNLSEKENSEVEF | 78 |
| | | \|\|+ \|\| \|\|++ \| \|\|+ \| \|\| + \|\|\| \|\|\|+ \|\|\|\|\|\|+\|+\|\| \| ++\|\|+ | |
| Sbjct: | 1 | SSLLKSLNELRENGEFCDVTLVVGGKEFPAHKAVLAACSPYFKALFSGNFKESDSSEITL | 60 |
| Query: | 79 | QCIDETALQAIVEYAYTGTVFISQDTVESLLPAANLLQIKLVLKECCAFLESQL | 132 |
| | | + +\|++\|+ \|\|\| + \|+++ \|\| \|\| \|+ \|\|\| ++ +\| \|\| \| | |
| Sbjct: | 61 | DDVSPEDFEALLEFIYTGELIITEENVEELLELADKLQIPSLVDKCEEFLIKNL | 114 |

TABLE 12F
Domain Analysis of NOV12

gnl|Smart|smart00225, BTB, Broad-Complex (SEQ ID NO:132)
Length = 96 residues, 100% aligned
Score = 99.8 bits (247), Expect = 4e−22

| | | | |
|---|---|---|---|
| Query: | 36 | DIILRVGDVKIHAHKVVLASVSPYFKAMFTGNLSEKENSEVEFQCIDETALQAIVEYAYT | 95 |
| | | \|+ \| \|\| \| \|\|\|\| \|\|\|+\|\|\|\|\|\|+\|+ + \| + \|\|+ + +\|++ + \|\| | |
| Sbjct: | 1 | DVTLNVGGKKFHAHKAVLAAHSPYFKALFSSDFKESDKSEIYLFDVSPEDFRALLNFLYT | 60 |
| Query: | 96 | GTVFISQDTVESLLPAANLLQIKLVLKECCAFLESQ | 131 |
| | | \| + \| ++ \|\| \|\| \|+ \|\|\| +++ \| \|\| | |
| Sbjct: | 61 | GKLDIPEENVEELLELADYLQIPGLVELCEEFLLKN | 96 |

TABLE 12G
Domain Analysis of NOV12

gnl|Smart|smart00612, Kelch, Kelch domain (SEQ ID NO:133)
Length = 47 residues, 97.9% aligned
Score = 69.7 bits (169), Expect = 4e−13

| | | | |
|---|---|---|---|
| Query: | 435 | IFVVGGHNGVSHLSSIERYDPHQNQWTVCRPMKEPRTGVGAAVIDN | 480 |
| | | I+V+GG NG L S+E YDP N+WT M PR+G G AVI+ | |
| Sbjct: | 2 | IYVIGGFNGGQRLKSVEVYDPETNKWTPLPSMPTPRSGHGVAVING | 47 |

Apoptosis or programmed cell death is an essential process in metazoan development and homeostasis that is carried out by caspases. The DIABLO protein (direct IAP binding protein with low pI) performs a critical function in apoptosis by eliminating the inhibitory effect of IAPs (inhibitor of apoptosis proteins) on caspases (1). This protein is also known as Smac for second mitochondria-derived activator of caspase. DIABLO/Smac is normally a mitochondrial protein but is released into the cytosol when cells undergo apoptosis. Mitochondrial import and cleavage of its signal peptide are required for DIABLO/Smac to gain its apoptotic activity. In addition, overexpression of DIABLO/Smac has been shown to increase cellular sensitivity to apoptotic stimuli (2).

The protein described in this invention is homologous to the DIABLO/Smac protein and is therefore predicted to play a role in apoptosis. It contains a BTB/POZ domain as well as five copies of the kelch motif. The BTB/POZ domain has been shown to mediate homomeric dimerisation and in some instances heteromeric dimerization (3). Kelch is a 50-residue motif, named after the Drosophila mutant in which it was first identified (4). The known functions of kelch-containing proteins are diverse. The gene described in this invention maps to chromosome 14 and based on its expression pattern may contribute to a number of human diseases such as cancer, inflammation/autoimmune diseases, metabolic diseases and CNS disorders, among others. In addition, because the novel DIABLO-like protein is likely to play a role in regulating apoptosis, this gene may be useful as a diagnostic or prognostic tool and in gene therapy.

The protein similarity information, expression pattern, and map location for the NOV12 protein and nucleic acid disclosed herein suggest that NOV12 may have important structural and/or physiological functions characteristic of the DIABLO family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: cancer, trauma, bacterial and viral infections, regeneration (in vitro and in vivo), fertility, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, Von Hippel-Lindau (VHL) syndrome, tuberous sclerosis, endocrine disorders, Alzheimer's disease, stroke, hypercalceimia, Parkinson's disease, Huntington's disease, cerebral palsy, epilepsy, multiple sclerosis, ataxia-telangiectasia, leukodystrophies, behavioral disorders, addiction, anxiety, pain, neuroprotection and other diseases, disorders and conditions of the like.

The novel nucleic acid encoding the DIABLO-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV12 protein has multiple hydrophilic regions, each of which can be used as an immunogen. In one embodiment, a contemplated NOV12 epitope is from about amino acids 5 to 7. In another embodiment, a contemplated NOV12 epitope is from about amino acids 10 to 15. In other specific embodiments, contemplated NOV12 epitopes are from about amino acids 80 to 90, 130 to 135, 140 to 145, 170 to 180, 190 to 192, 198 to 205, 220 to 270, 295, 320, 340 to 370, 400 to 415, 420 to 470, 490 to 500.

NOV13

A disclosed NOV13 nucleic acid (designated CuraGen Acc. No. CG56195-01) encodes a novel HRPET-1 related protein-like protein and includes 1970 nucleotides (SEQ ID NO: 45) is shown in Table 13A.

TABLE 13A
NOV13 Nucleotide Sequence

(SEQ ID NO:45)

TTTTTTTTTTTTTTTCTCTATACAAGGCTGTTTATTTCTGTACAAAACCATGTTTCTATTTTACACAAAG
AACACCCCACCCTTTCCCCTCACACCAGCACCCTAACCCTGGGGAGCATCCCCCAGGAGGAGGGGGCTGA
AGGAGGCCCCACCCCCCAGGCCCTAGCTTCTGCCTGCCCTGGCTGGGCCAGCCTGAGTGCCACTGTAAAG
AAAATAAATAAGGAGGCTCAGGCAGAATCTGTGTTGGACCAGGCAGAATCTGTGTTGGACCAGCCAGACT
CCAGCCCAGCCCAGTGGCCAGGCAGCTTGGCCCTCAGAGGGTGGGCAGGATGTGGAATGTCAGTTCATGA
ACCGTGTAGTTATATGGAGACCCCGCCCTGGAGGCCTTAGCTGCCAGGGTTACAAGTAGGTGTCCTCACT
CTCTTGGGACGTCAAGCTCTCCTGGGAGCGGTGGTGGGCTGAGACCTGGGGAGCCAAATCCTGAGGGGCT
GAGTCCTTGGGGGCTGAGTCCTTCGGGGGCACATGCTGTGGGGGACATGCATCTCCTGCAGCGGCCACAA
CCATGGCTTGATTTGGGGCTGGGGGCTTCTCCAGCTGCCCTCTCCCCTTCATCTGTTTCCGCTGCTCCTT
CTGGGCCTGCTTGGGTGGCTTGGGCTTGGCTTTGGAGCCAGGGAGGGGGGCATCTAGGGGCAGGCGGATG
GATGGTGAAGGTTGTAGGGCAGGCCGGGGACCAGGTTCTGCATCCAAGATAGCCTTGGCACCATGCAGCC
TGGGCGGGGAGCGGCACTGCAGCTCACCCCGGGTCTCCTGCCAGCGCCGCAGCTGAATGAGGTGTTCGCG
CTCAATCTGGCGCTCTGTCACGGGCAACTCCACCACCTCCTGGACCAGAAAGGCCTCCTGCATGATCTTG
GGGCTGAGGCTCCGCAGTCGCTCGATGGTCTCGTACTGGCCCTGGCAGGCTTTGACCTTCTCAGGGGAGC
CCAGCGCGTGCTTCAGCAGCACCAGCCCCACCCGGAGATGATCTTGACCCCTTCACAGAAAGAACATGTC
CCAGACACGCAGCACAGAGCTCCAGGGCAAGGTTCGGGAGAAGGCGCACATGAACCATTCTGTCATATAG
AGGAGCGGGTCGATCTTCTGACGGCTGAGGTGCTTGTGGGCCACCGGCGACACCTTCTGCAACAGCGAGA
AAAGGATCTCCCCGTCCAGCTGGATCGCCTCCAGTTTCTCGCTGTAGTAGCCGGGCAGGTACTTCTCACA
GATCTGTACCAGGCACCAGAAGCCTTGCTCAGCAGGCATATGCATGAGCAAGACAGCGGCAATGGGCGCC
TGGGCCTGGCAGTAGCCCTCCTCGGGCCGGTACAGCGTGTAGGCCTTCAGCACACGGAATAGGTCCTGCT
GGCCGTGGCCCCCCCGGGACACAAACATCTCATGGAATGGGAACTGCCGGTGCAGGTCACGCTCAATCAC
GTCCAGCCACTTGGGGTCCCCAGGGGACATGTCCAGCTCGTCAAACTTTCCAGGGTTCTGCTGTAACTTC
ACCTTGCCTCCTGACAGGTACTGCCAAGCACGGCCCCGCAGAGAAGGCGGGATGCCCTTTTGGCACCGCA
GACGAATCTTTTTGTGCTTCTTGGCCATCCATTTGTCCCAGTTGTTGAGCATGTCCAGCCACTTGGACTC
CCTCTGCCTCAGCACCTCCAGGGGGACTTCCTCCAGCGCGCCCTCGGCGCCCTGCGAGCCCACGATGAAG
CCGAACTTGTCGATGCGGCGCTCGGCGAAGCCGTTGGCCTCCGAGTCAGACCCGAGAGAGCTGAGTTCGT
CGGTCGTTGCGGCGTCGGGGCCCTGGGCCAGGCTCTCCCGGGTTCCCGACAGGCTTTCCCCGGCCGCGGG
CGCGCGCGGCCCATTCTCTCCGTTGCTCTTCGCCATCCCAGCCGCGCCCGCCGCCTGAGCTCCAGCGGCC
ACCTCAGCCG

The nucleic acid sequence of NOV13 maps to chromosome 22 and has 891 of 1228 bases (72%) identical to a gb:GENBANK-ID:AK023192|acc:AK023192.1 mRNA from Homo sapiens (Homo sapiens cDNA FLJ13130 fis, clone NT2RP3002972, weakly similar to Halocynthia roretzi mRNA for HrPET-1) (E=0.0).

A NOV13 polypeptide (SEQ ID NO: 46) is 508 amino acid residues and is presented using the one letter code in Table 13B. Signal P, Psort and/or Hydropathy results predict that NOV13 is likely to be localized at the plasma membrane with a certainty of 0.7000. In another embodiement, NOV13 is likely to be localized to the nucleus with a certainty of 0.3000, the endoplasmic reticulum (membrane) with a certainty of 0.2000 or the mitochondrial inner membrane with a certainty of 0.1000.

TABLE 13B
NOV13 protein sequence

| | |
|---|---|
| MAKSNGENGPRAPAAGESLSGTRESLAQGPDAATTDELSSLGSDSEANGFAERRIDKFGFIVGSQGA | (SEQ ID NO:46) |
| EGALEEVPLEVLRQRESKWLDMLNNWDKWMAKKHKKIRLRCQKGIPPSLRGRAWQYLSGGKVKLQQN | |
| PGKFDELDMSPGDPKWLDVIERDLHRQFPFHEMFVSRGGHGQQDLFRVLKAYTLYRPEEGYCQAQAP | |
| IAAVLLMHMPAEQAFWCLVQICEKYLPGYYSEKLEAIQLDGEILFSLLQKVSPVAHKHLSRQKIDPL | |
| LYMTEWFMCAFSRTLPWSSVLRVWDMFFCEGVKIIFRVGLVLLKHALGSPEKVKACQGQYETIERLR | |
| SLSPKIMQEAFLVQEVVELPVTERQTEREHLTQLRRWQETRGELQCRSPPRLHGAKAILDAEPGPRP | |
| ALQPSPSIRLPLDAPLPGSKAKPKPPKQAQKEQRKQMKGRGQLEKPPAPNQAMVVAAAGDACPPQHV | |
| PPKDSAPKDSAPQDLAPQVSAHHRSQESLTSQESEDTYL | |

The NOV13 amino acid sequence have 438 of 438 amino acid residues (100%) identical to, and 438 of 438 amino acid residues (100%) similar to, the 438 amino acid residue ptnr:SPTREMBL-ACC:O76053 protein from Homo sapiens (Human) (WUGSC:H_DJ130H16.2 PROTEIN) (E=4.3e⁻²⁴²).

NOV13 is expressed in at least the following tissues: bone marrow, brain, bronchus, dermis, epidermis, heart, kidney, liver, lung, lymph node, lymphoid tissue, mammary. This information was derived by determining the tissue sources of the sequences that were included in the invention including but not limited to SeqCalling sources, Public EST sources, Literature sources, and/or RACE sources. In addition, the sequence is predicted to be expressed in the following tissues because of the expression pattern of (GENBANK-ID: gb:GENBANK-ID:AK023192|acc:AK023192.1) a closely related Homo sapiens cDNA FLJ13130 fis, clone NT2RP3002972, weakly similar to Halocynthia roretzi mRNA for HrPET-1 homolog in species Homo sapiens: testis, ovary, colon, parathyroid, thyroid, bone, spleen, stomach, cervix, adrenal gland, head-neck.

NOV13 also has homology to the amino acid sequences shown in the BLASTP data listed in Table 13C.

TABLE 13C
BLAST results for NOV13

| Gene Index/ | Protein/ | Length | Identity | Positives | |
|---|---|---|---|---|---|
| Identifier | Organism | (aa) | (%) | (%) | Expect |
| gi\|13994322\|ref\| | EBP50-PDZ | 508 | 485/508 | 485/508 | 0.0 |
| NP_114143.1\| | interactor of 64 | | (95%) | (95%) | |
| (NM_031937) | kD [Homosapiens] | | | | |
| gi\|17390711\|gb\| | Similar to | 500 | 445/508 | 457/508 | 0.0 |
| AAH18300.1\|AAH18300 | EBP50-PDZ | | (87%) | (89%) | |
| (BC018300) | interactor of 64 | | | | |
| | kD [Mus | | | | |
| | musculus] | | | | |
| gi\|3212997\|gb\| | match to ESTs | 438 | 415/438 | 415/438 | 0.0 |
| AAC23434.1\| | AA667999 | | (94%) | (94%) | |
| (AC004997) | (NID:g2626700), | | | | |
| | AA165465 | | | | |
| gi\|10435007\|dbj\| | unnamed protein | 533 | 261/375 | 317/375 | e−155 |
| BAB14454.1\| | product [Homo | | (69%) | (83%) | |
| (AK023192) | sapiens] | | | | |
| gi\|15076925\|gb\| | unknown WZ3-85 | 537 | 259/375 | 316/375 | e−150 |
| AAK82984.1\|AF285112 | [Mus musculus] | | (69%) | (84%) | |
| _1 (AF285112) | | | | | |

The homology of these sequences is shown graphically in the ClustalW analysis shown in Table 13D.

[image]

[image]

[image]

Tables 13E and 13F list the domain description from DOMAIN analysis results against NOV13. This indicates that the NOV13 sequence has properties similar to those of other proteins known to contain these domains.

TABLE 13E
Domain Analysis of NOV13

gnl|Smart|smart00164, TBC, Domain in Tre-2, BUB2p, and Cdc16p. Probable
Rab-GAPs (SEQ ID NO:139)
Length = 218 residues, 95.9% aligned
Score 189 bits (481), Expect = 3e-49

| | | | |
|---|---|---|---|
| Query: | 108 | CQKGIPPSLRGRAWQYLSGGKVK-LQQNPGKFDEL--DMSPGDPKWLDVIERDLHRQFPF | 164 |
| | | +\|\|\|\|\|\|\|\|\| \|+ \| + \| \| + + \| +\| \| \|\|+\|\| \| \|\|\| | |
| Sbjct: | 1 | VRKGIPPSLRGEVWKLLLNAQPKNLSNDKDLYSRLLRQTAPKKKSTLKQIEKDLPRTFPE | 60 |
| Query: | 165 | HEMFVSRGGHGQQDLFRVLKAYTLYRPEEGYCQAQAPIAAVLLMHMPAEQ-AFWCLVQTC | 223 |
| | | \| +\| \|+ \| \|\|\|\|\|\|++\| \|\| \|\|\|\| +\|\| \|\|+ \|\| \|+ \|\|\|\|\|\|++ | |
| Sbjct: | 61 | LPPFQFKGPG-QESLRRVLKAYSIYNPEVGYCQGMNFLAAPLLLVMPDEEDAFWCLVKLM | 119 |
| Query: | 224 | EKYLPGYYSEKLEAIQLDGEILFSLLQKVSPVAHKHLSRQKIDPLLYMTEWFMCAFSRTL | 283 |
| | | \|+\|\|\| +\| \| + \| +\| \|\|\|\|+ \| +\|\|\| + \|\|\| \|\| \|\|+ \|+\| \| | |
| Sbjct: | 120 | ERYLPNFYLPDLSGLHADQLVLDSLLQEYLPDLYKHLQEKGIDPSLYALRWFLTLFAREL | 179 |
| Query: | 284 | PWSSVLRVWDMFFCEGVKIIFRVGLVLLKH 313 | |
| | | \| \|\|\|+\|\|+ \| \|\| + +\|\|+ \| +\|\| | |
| Sbjct: | 180 | PLEIVLRIWDVLFAEGSEFLFRIALAILKL 209 | |

TABLE 13F
Domain Analysis of NOV13

gnl|pfam|pfam00566, TBC, TBC domain (SEQ ID NO:140)
Length = 217 residues, 95.4% aligned
Score = 163 bits (413), Expect = 2e-41

| | | | |
|---|---|---|---|
| Query: | 110 | KGIPPSLRGRAWQYLSGGKVKLQQNPGKFDELDMSPGDPKWLDV---IERDLHRQFPFHE | 166 |
| | | 51 +\| \|\|\|\| \|+ \| \| + +\| + ++ \|\|++++++++++\| \|\|+\|\| \| \|\| | |
| Sbjct: | 3 | GGVPSSLRGYVWKLLLGAQ-ELNNDKDEYIELLNKHKPETVQDQLDQIEKDLSRTFPDDI | 61 |
| Query: | 167 | MFVSRGGHGQQDLFRVLKAYTLYRPEEGYCQAQAPIAAVLLMHMP-AEQAFWCLVQICEK | 225 |
| | | \| \| \| \|+\|+\|\|+ \|+ \|\| \| \| + \|\|+ + \|\|\|\|\|\| ++ + | |
| Sbjct: | 62 | FFHSNEPPSIAQLRRLLRAYSWKNPDLGYVQGMNDILSPLLLFLKDEEQAFWCFTKLMDN | 121 |
| Query: | 226 | YLPGYYSEKLEAIQLDGEILFSLLQKVSPVAHKHLSRQKIDPLLYMTEWFMCAFSRTLPW | 285 |
| | | \|\|\| \|++ \| \| +\| \|\|+++ \| + \|\| +\| \|++ \|\|+ \|+\| \|\| | |
| Sbjct: | 122 | YLPQYFTNDLSGSNEDLRVLDSLVKESLPELYSHLKKQGSTLLIFAFPWFLTLFARELPL | 181 |
| Query: | 286 | SSVLRVWDMFFCEGVKIIFRVGLVLLKH 313 | |
| | | \|\|\|+\|\|\| \| \| + \| \| +\|\| | |
| Sbjct: | 182 | EIVLRIWDMLFTYGSHFLIFVALAILKL 209 | |

NOV13 is highly conserved across species, among C. elegans, Drosophila, mouse and human. It's predicted to be membrane associated. The high conservation in primary sequences indicates that it has important biological functions, although currently unknown. The HRPET-1 related protein also shows homology with plant adhesion molecules, suggesting that the HRPET-1 related protein is likely a cell adhesion molecule involved in cell interaction and migration.

The protein similarity information, expression pattern, and map location for the NOV13 protein and nucleic acid disclosed herein suggest that it may have important structural and/or physiological functions characteristic of the cell adhesion molecule family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, diabetes, Von Hippel-Lindau (VHL) syndrome, pancreatitis, fertility, endometriosis, xerostomia, cirrhosis, hemophilia, hypercoagulation, idiopathic thrombocytopenic purpura, autoimmume disease, allergies, immunodeficiencies, graft versus host disease, lymphedema, hemophilia, hypercoagulation, Alzheimer's disease, stroke, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, systemic lupus erythematosus, asthma, emphysema, scleroderma, ARDS, psoriasis, actinic keratosis, acne, hair growth/loss, allopecia, pigmentation disorders, endocrine disorders, renal artery stenosis, interstitial nephritis, glomerulonephritis, polycystic kidney disease, systemic lupus erythematosus, renal tubular acidosis, IgA nephropathy, Lesch-Nyhan syndrome and other diseases, disorders and conditions of the like.

The novel nucleic acid encoding the HRPET-1 related protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV13 protein has multiple hydrophilic regions, each of which can be used as an immunogen. In one embodiment, a contemplated NOV13 epitope is from about amino acids 2 to 70. In another embodiment, a contemplated NOV13 epitope is from about amino acids 90 to 120. In other specific embodiments, contemplated NOV13 epitopes are from about amino acids 125 to 200, 210 to 215, 220 to 230, 310 to 320, 380 to 390, 390 to 398, 410 to 425 and 480 to 500.

NOV14

A disclosed NOV14 nucleic acid (designated CuraGen Acc. No. CG55790-02) encoding a B7-H2-like protein includes 8270 nucleotides (SEQ ID NO: 47) and is shown in Table 14A. An open reading frame was identified beginning with an ATG initiation codon at nucleotides 24-26 and ending with a TAG codon at nucleotides 1443-1445. A putative untranslated region downstream from the termination codon is underlined in Table 14A, and the start and stop codons are in bold letters.

TABLE 14A
NOV14 Nucleotide Sequence

(SEQ ID NO:47)

GGCCCGAGGTCTCCGCCCGCACCATGCGGCTGGGCAGTCCTGGACTGCTCTTCCTGCTCTTCAGCAGCCT
TCCAGCTGATACTCAGGAGAAGGAAGTCAGAGCGATGGTAGGCAGCGACGTGGAGCTCAGCTGCGCTTGC
CCTGAAGGAAGCCGTTTTGATTTAAATGATGTTTACGTATATTGGCAAACCAGTGAGTCGAAAACCGTGG
TGACCTACCACATCCCACAGAACAGCTCCTTGGAAAACGTGGACAGCCGCTACCGGAACCGAGCCCTGAT
GTCACCGGCCGGCATGCTGCGGGGCGACTTCTCCCTGCGCTTGTTCAACGTCACCCCCCAGGACGAGCAG
AAGTTTCACTGCCTGGTGTTGAGCCAATCCCTGGGATTCCAGGAGGTTTTGAGCGTTGAGGTTACACTGC
ATGTGGCAGCAAACTTCAGCGTGCCCGTCGTCAGCGCCCCCCACAGCCCCTCCCAGGATGAGCTCACCTT
CACGTGTACATCCATAAACGGCTACCCCAGGCCCAACGTGTACTGGATCAATAAGACGGACAACAGCCTG
CTGGACCAGGCTCTGCAGAATGACACCGTCTTCTTGAACATGCGGGGCTTGTATGACGTGGTCAGCGTGC
TGAGGATCGCACGGACCCCCAGCGTGAACATTGGCTGCTGCATAGAGAACGTGCTTCTGCAGCAGAACCT
GACTGTCGGCAGCCAGACAGGAAATGACATCGGAGAGAGAGACAAGATCACAGAGAATCCAGTCAGTACC
GGCGAGAAAAACGCGGCCACGTGGAGCATCCTGGCTGTCCTGTGCCTGCTTGTGGTCGTGGCGGTGGCCA
TAGGCTGGGTGTGCAGGGACCGATGCCTCCAACACAGCTATGCAGGTGCCTGGGCTGTGAGTCCGGAGAC
AGAGCTCACTGGTGAGTTTGCCGTGGGAAGCAGCAGGTTCTGGGGGGCCCAGGGGAGGCTTGGCTGCCAG
CTGTCTTTCAGAGTTTCAAAAAACTTTCAAAAGGCAAAAGTCCCTTGCCTTGAACAACTGTTGTTCCTGG
AGACGCAGCGAAGCCCTCGATGGTGCGCATGGCATTTCCTGCAGCCTCCCCTTGGCATGGGATGGCATCC
TGGTGTGCACTTTGTCACACTGCGATGGGATTTTCCCAACATGCACAGAAGCAGAGAGACGAGTGCTAGA
CCCCCGCGCTCCCCAGTGCCCAGCCCCGACCAGGGTGTCCAGGGCGGGTCCAGGCACCGGCGCCCAGCCC
CCATGGGGTGTCCGGAGTGGGTCCAGGCACCGGCGCCCAGCCCCCGTGGGGTGTCCAGGGCGGGTCCAGG
CACCGGCGCCCAGCCCCTGTGGGGTGTCCGGAGTGGGTCCGGGCACCGCCAGCTTCTCTCTGTGGCAGCC
ACTCCTGCAGCTCTCGTTTGCCCCTCAGTTCCAGGAGCAACATAGATGTGGATTCCTGTCCAATTTGGGA
AAAATGTCCACACACGGTCACCCACCTGGCAGGTGCCTCTGGCTGCAAGGGGCGCTGGGCTTCGCAGGCA
GGCCAGCCGGGCTCCCCGCCATGGGCCAGGATCCCCTCCGAGCCCTGTTTGCCGCCCAGGAGAAGGGGTT
CCCCGGGGACAGTGGGCTCAGGGTGTGCGCAGCCACCATGCTGTGGTGTCACCTGTGGACCCAGGCGAGC
TGATGGCCGACCGCAGAAACGCACTTCCAAGGCCAGGTCGGCCCATCCAGATGATGCAGGAACACAGCTT
GCTAAAAACACGGCCGGCCTGTTCCCGTCGGAGCCAGTCGAAGTTCCCTGAACAGGCCGCTGTTTCCGAA
GCTTTAAACCCTGTGTTTCCACCAAGCTGAGTCCTGAGAAAACCGACGTCTGCCTGCAGAAGGGAAAGGG
GTGCTTCATGTTCCTCTCTCTCCTTCATCTCCCTTCCAAGGCCACGTTTGACCGGAGCTCACCGCCCAGA
GCGTGGACAGGGCTTCCGTGAGACGCCACCGTGAGAGGCCAGGTGGCAGCTTGAGCATGGACTCCCAGAC
TGCAGGGGAGCACTTGGGGCAGCCCCCAGAAGGACCACTGCTGGATCCCAGGGAGAACCTGCTGGCGTTG
GCTGTGATCCTGGAATGAGGCCCTTTCAAAAGCGTCATCCACACCAAAGGCAAATGTCCCCAAGTGAGTG
GGCTCCCCGCTGTCACTGCCAGTCACCCACAGGAAGGGACTGGTGATGGGCTGTCTCTACCCGGAGCGTG
CGGGATTCAGCACCAGGCTCTTCCCAGTACCCCAGACCCACTGTGGGTCTTCCCGTGGGATGCGGGATCC
TGAGACCGAAGGGTGTTTGGTTTAAAAAGAAGACTGGGCGTCCGCTCTTCCAGGACGGCCTCTGTGCTGC
TGGGGTCACGCGAGGCTGTTTGCAGGGGACACGGTCACAGGAGCTCTTCTGCCCTGAACGCTCCCAACCT
GCCTCCCGCCCGGAAGCCACAGGACCCACTCATGTGTGTGCCCACAAGTGTAGTTAGCCGTCCACACCGA
GGAGCCCCCGGAAGTCCCCACTGGGCTTCAGTGTCCTCTGCCACATTCCCTGGGAGGAACAATGTCCCTC
GGCTGTTCCGGTGAAAAGTTGAGCCACCTTTGGAAGACGCACGGGTGGAGTTTGCCAGAAGAAAGGCTGT
GCCAGGGCCGTGTTTGGCTACAGGGGCTGCCGGGGCTCTTGGCTCTGCAGCGAGAAAGACACAGCCCAGC
AGGGCTGGAGACGCCCATGTCCAGCAGGCGCAGGCCTGGCAACACGGTCCCCAGAGTCCTGAGCAGCAGT
TAGGTGCATGGAGAGGGTATCACCTGGTGGCCACAGTCCCCCTTCTCACCTCAGCAATGATCCCCAAAGT
GAGAGGTGGCTCCCCCGGCCCCCACCACCCTCAGCAGCCCCACCCCACTCAACCCTGAGGGTCCCCAGGG
TCCTGATGAAGACCTCCGACCCCAGCGCCAGGCTCCTCGGAGCCCAACAGTCCCAAGGGGGCAGGAGACG
GGGTGGTCCAGTGCTGAGGGGTACAGCCCTGGGCCCTGACCAGCCCCGGCACCTGCCATGCTGGTTCCCG
GAATGAATCAGCTGCTGACTGTCTCCAGAAGGGCTGGAAAGGATGCTGCCAGGTGACCCGAGGTGCACTC
GCCCCAGGGAGATGGAGTAGACAGCCTGGCCTGGCCCTCGGGACACATTGTCTGCCCCGGGGCTATGGGC
AAATGCCCCTCCTTCTTACTTCCCAGAATCCCCTGACATTCCCAGGGTCAGCCAGGACCTGTTACAGCCC
TGGTCACTTGGAACTGACAGCTGTGTGAGGCCTGCACTTCTCAGACCCAGACTTAGAACAAAAGGAGGAG
TGAGGACTCAAGGCTACAATGAGGTTCCAGTACTTGTTACAAGAAATTGGTTTTCTGCAAAAAAAGTCCC
TACCTGAGCCTTTAGGTGAATGTGGGATCCACTCCCGCTTTTAACATGAAAGCATTAGAAGATGTGTGGT
GTTTATAAAAGAACAGTTGTCATCACCGGGCATTGATTGGCAGGGACAAGGAGCTGCTTGGGTGTGGAAA
GTTGGGGCGTTGGAAAGTGGGCTGTGGTGCCCATTTGCAGTGACTGTGAAGTGACTCCAGGACGGACCTG
CGGGGGCACCCAGAGGTCCTAAGCCCCAGGACTGAGGGTCGTGCATCACCACTCGGGTGTCCCGGGAGGT
GCCCTGGGCCCGGGGACCTCACAGGCAGGACGGCGACACTAATGCAGGGAGAGGGAGTCTGGCCCCAGCT
TTTCCTATCAGAGGCGATTTTCCTTCACCAGGGGATGGGCAGGAAAGAGGCAGGGGCCCCAGAAGCTTCT
GTCCCTCATGCCTGAGGGCACGGGGGACACTTGGAGGCTGCTGTCACCACTGTGCGTCCAAGGCCATGCT
CTCTGCGGGTCAGTGCCTGAGTCTCGCCTCCCTGCTGGTCCCTGAAGCCCCCTCAGAAGCCCTGCCTGTC
ACGTCGGCATTTGTGAGACCTACCCTGTAACGCCTGCCCCTCTCAGCCCAACATCAGCTTCCTCTTTCTC
CCTTGCTGTAGACAGGCTGGATTCCAGTGTTGGGACAGCCATCTCCAGAAACCTGACTTAAGAGAGTAAG
ATGCAAATCGTGCCTGTATCCAGTGGCTTTGGTGGGTGCAGGGAGTCTTGGGCACAGCCAGCTCAGCTGT
CTGTGGTATGAGCAGGAACAGGTGCCACTCCTGCTCAGGGGACCCTGCCCTACACCAGGCTGTTCCGTCC
CCCTGGAGGACATGGGGCCAGGTCTGGAGGCATTTTGGGTTGTCACAGCTGGGGGCTGTTCCTCGGCTTC
AGCGGGTGGAAGCCTCAGATGCTGTTCAACATCTTCTGGACACGGGAGGCCCCGACAGAGAGAAGCGTCC
ACCCGCAAGTCCACAGTCTGAGGTCTCCCCTCAGAGACCCTGCCCTGCACACCCACCTCCAGCCAAAGGT
CCTGCCTGCCCCAGGGCTCAGGGGAACCTTGCCGGTCTGTGGAACAGGAGAGGGGACTCTCGCCAGCTGC
ACCACCCTGCACGTAGTAGGTGTGCGGTAAACATCCACCAGGGAGGCTCCAGTCAAGGCTGGCAGATGGG
GCGGTCCATCCCTAGGGCAGGTGACAGAAGGGAAAAGGCTGCCTGCTGGCCCCCGAGCCAGGTAGCACAT
GCTTGTGCCTCAGTTTCCCCTCCTGTAAAGTGAGGCGCTGGATCCAGGTTCTGTCTACTGGGCTCTGCAG
CTTGGACGCTCCTAAGACCAAGCGACCCACCCTGGGGAGGGCAGCTATGGCTTTGGAATAGCTGTCCAGG
CCCGGGTGCCTCCAAGACGGCCACCACACCCTGCCTGTGCTGCAGGGGTGCAGGGGTAAGGGGCAAGACT
CCAGAGGCCTCCTCTCTGCATCTCCTTGTCTTCAGTGGCCGGAGGTGAGGCCTGAGCTCAGGGGAGGGGC
TTCTGCCACGAACCCTATGGCGGGGCACAGCACACTTTTCCCAGGGAGGACCCCTGGGCCCCCTGCATTA
TCCCCAGCGGAGTGTGGGGTCACCTTCCAAGAGCGACATTGAGAAGCTCCAGCTCTAGGAGTGTGCAGAC
TCTTAACCAGGCAGGCCCAGGCCCTGGGGCACACAAAGGCGGGGCCTGCTCTCCCCAGCTGCCCCTGCCA
ATGGGGGCTGGACTGTCCTACCCTCCTCCCTTCTACCTCCCCACTGTCTTCCCTCTCCACTGTCACCACT
GCCTCCCTCTTCCACTGTCCTCCATGCACTGCCCTCCCTCCACCTTCCCCCACCCCCACCACTCCCCATG
CTGTCCCCAGGCTCCCCCCGCTCTCCCCCCTCCCCACTGTCCCCCTCCCCATGCTGTACCCAGCTCACCC
CGCTCTCCCCTCTCCCCACTGTCCCCCCTCCCACTCCCCATGCTGTCCCCAGCTCACCCTACATGGACTT
GGCGATGTCCTTCCATGGCTCACCGGTCTGAATTTCCATGATGAGCCGGGCCTGCAGCTTTGCTCCCCTA
TCCCTGCCCAGCCTGCAGCTGTCCATGCAGGGAGCGAGCTCCAGCACCTGCGGAGTCCTTCCGTGGGGGC
CTCTCCGTGCCACAGCAGCCAGGGACCTCAGGTGCCTGTGCATGACACCACCGCCCATCCTCATCCTGAG
CCAGCCTCTCAGGATCAGGACTTGGTTTGGCGGCGTTAACCTTAGAGCCTGCAAGGGGCTTCCTCCTGGT
GGGTCTGGCCGTAGCCTGGGGAGGCCACAGCTCCAGGCCACTCCAGACCTCCCTTCCTCTGGGCCTTCCA
TGTGGTGGCAACCACCGCAGCTGTAAGGGAGGGAAAATGGAGCGTTTGTTCTCGCGCTGGGCTGGGGTCT
GGGGGAAGCCATGGGCGTGAAGACTGGAGTATTATTTGATGGAGAAGCGGCCACTCCTGGAGACCGGCGG
CAAACACAGAAGCACAGCGTGGAAGGTGCTGGTGTCAGCCCACACGGGTGATGGGGTCAGACTCAGGAGT
CACACTCAGGAGTCACCAGGCTCAAAGGGCCCAGGCACCGCAAGTCCTGCTCAGCCCCAGACACAATGCA
TTCCTGTTGCCCTCGCCCTCAGCCAGGCCCCACGCAGGCCAGGGAGCACTGGCAAAGCTTGGCAACCCTC
TGGGGGCCAGCCTTCATCCAGGCCGAAGGTGGTCAGTGGCCCACCATGGCCCAGGTAGAAAACTCACGGA
TTAAGATTTCATGCCCGACTCCAAAGGCAAGAGACTTTATTATTTTATTTTTTTTGAGCCAGAGTATCGC
TCTGTCACCTAGGCTGGAGTGCAATCTCTGCTCATTGCAACATCTGCCTCCCGAACTCAAGCAATTCTGC
CTCAGCCTCCCAAGTAGCTGGGATTACAGGTGTGCGCCACCATGCCCAGGTAATTGTATTTTTAGTAGAG
ACAGGGTTTCACCATGTTGGTCAGGCTGCTTTCAAACTCCTGACCTCAAATGATCTGCCCACCTCGACCT
CCCAAAGTGCTGGGATTACAGGTGCGAGCCACCGCACCTGGCTACCAGACACTTCAGAGTTACAGGTTAG
TTTTTCTTTTTCTTTTATTTTTTTTTTTTTGGCGGAGGTGCAGGGGGAGTTAAACAAACAAACAAAATAA
ACAGGCCGGGTGCGGTGGCTCATGCCTGTAATCCCAGCACTTTAGGAGGCCTAGGTGGGTGGATCACGAG
ATCAGGGGTTCAAGACCAGCCTGGCCGAGATGGTAAAACCCCGTCTCCACTAAAAATACAAAAATTGGCC
AGGCACGGTGGCTCACACCTGTAATCCCAGTACTTTGGGAGGCTGAGGTGGGCAGATCACCTGAGGTCAG
GAGTTCAAGACCAACCTGACCAACATGGAGAAACCCCATCTCTACTAAAAATACAAAATTAGCCAGGTGT
GGTGGTGCATGCCTGTAATTCCAGCTACTCGGGAGGCTGAGGCAGGAGAATTGCTTGAACCCAGGAGGCA
GAGGTTGCAGTGGGCCAAGATGGCGCCATTGCACTCCAGCCTGGGAACAAGAGCGAAACTCTGACTAAAA
AAGAAAGAAAGAAAGAAAAAAATTAGTTGGGCACGGTGGCAGGCGCCTGTAATCCCAGGTACTCAGGAGG
CTGAGGCAGGAGAATTGCTTGAACCCGGGAGGCAGAGGTCGCAGTGAGCCGAGATTGCACCACTGCCCTC
CAGCCTGGGTGACAGAGCAAGACTCCGTCTAAAAAAAAAAAAAAAAAAAAATTGGATACATTGTAATACC
TCAAATACTTGTAAGTGAAGCACCCCAGTTCCCATAGAGCTGCCGCACTCAGAGGCTTCTGTAACCTGCC
TGCTCCCAGCATTCTATTTAGGGTCTGGTATGTCCAGAATTTGCAGACACAGCAATTCCTGCAGCAGCAG
TGCACCATGTGGAAGGGGCCCCATGACCAGCCCACTGTGAGCTCACACGTGATGACTGAGGCTTCTTCAC
ACAGCAGGGCTCTGGGTGTGATACCCAGGGCACACGCGTTTGCACAGGCACAGGCCACACAAGTTCTCAC
ATGCTCAGCCCCATAAGCCGTGCTGGACAGGCATGGCCATTTACACCCAGGATCCTGCTGAGAACAGCAA
CCAACTCACCACCCTCGCATCATGATCCTTGCCACACAGGGGCTCTGGTGGCTTTGGTGGCCTGGGCTGT
GGCTCTGCTGCCACCCACCTTGAGTGAAGATCCGGGTTCTCTGGGTGCTACTCAGCTGCTATGTGGGGAG
CTGGCCCCTGGGGTGATGAGGGCCCTTCCCAACCCGCCCTCAGCCCTTGGACAGCCAGGATCACCCGGGG
CTGTCTGCATACAGACTTCTCAGGGGAGTTCTCAGCTTGGACCCTTATCTCCCCAGAATCCTGGAACCTG
CTCCTTCTGCTCTCGTGACTGACTGTGTTCTCTATGCAACTTCCAATAAAACCTCTTCATTTGAAAGGAA
AAAAGTCTGCATTATCTGTTTAGGAAGGGAGAGAGTTCATATTGCAATCTTTTTTTTTTTAATAAAAATA
ATCTCAGCCTGGGCAACATGGTGAGACCCCATCTCTGTAAAACATTTTTAAAAAATTAGCCGGGTATGGT
GGCGCACACTTGTAGTCCCAGCTACTCAGGAGGCTGAAGCGGGAGGATCCATTGAACCTGAGAAGTCGAA
GCTGCAGTGAGCTGTGATTGTGCCACTGTACTCCAGCCTCGACAACAGAGTGAGACGCCGTCTCAAATAA
ATAAATACAT

The nucleic acid sequence of NOV14 maps to chromosome 21 and has 480 of 607 bases (79%) identical to a gb:GENBANK-ID:AP001753jacc:AP001753.1 mRNA from Homo sapiens (Homo sapiens genomic DNA, chromosome 21 q, section 97/105) (E=1.5e⁻¹¹⁷).

A NOV14 polypeptide (SEQ ID NO: 48) is 473 amino acid residues and is presented using the one letter code in Table 14B. Signal P, Psort and/or Hydropathy results predict that NOV14 contains a signal peptide and is likely to be localized at the plasma membrane with a certainty of 0.4600. In other embodiments, NOV14 is localized to the endoplasmic reticulum (membrane) with a certainty of 0.1000, endoplasmic reticulum (lumen) with a certainty of 0.10000 or the outside of the cell with a certainty of 0.1000. The most likely cleavage site for a NOV14 peptide is between amino acids 18 and 19, at: LRA-DT.

TABLE 14B
NOV14 protein sequence

(SEQ ID NO:48)

MRLGSPGLLFLLFSSLRADTQEKEVRAMVGSDVELSCACPEGSRFDLNDVYVYWQTSESKTVVTYHIPQNSSLE
NVDSRYRNRALMSPAGMLRGDFSLRLFNVTPQDEQKFHCLVLSQSLGFQEVLSVEVTLHVAANFSVPVVSAPHS
PSQDELTFTCTSINGYPRPNYVWINKTDNSLLDQALQNDTVFLNMRGLYDVVSVLRIARTPSVNIGCCIENVLL
QQNLTVGSQTGNDIGERDKITENPVSTGEKNAATWSILAVLCLLVVVAVAIGWVCRDRCLQHSYAGAWAVSPET
ELTGEFAVGSSRFWGAQGRLGCQLSFRVSKNFQKAKVPCLEQLLFLETQRSPRWCAWHFLQPPLGMGWHPGVHF
VTLRWDFPNMHRSRETSARPPRSPVPSPDQGVQGGSRHRRPAPMGCPEWVQAPAPSPRGVSRAGPGTGAQPLWG
VRSGSGHRQLLSVAATPAALVCPSVPGAT

The NOV14 amino acid sequence has 300 of 300 amino acid residues (100%) identical to, and 300 of 300 amino acid residues (100%) similar to, the 302 amino acid residue ptnr:TREMBLNEW-ACC:AAG01176 protein from Homo sapiens (Human) (TRANSMEMBRANE PROTEIN B7-H2 ICOS LIGAND) (E=7.3e⁻¹⁶⁰).

NOV14 is expressed in at least the following tissues: bone marrow, brain, thalamus, adipose, amygdala, bone, heart, kidney, lymphoid tissue, mammary gland/breast, ovary, pancreas, peripheral blood, prostate, thalamus, tonsils, urinary bladder, uterus, vulva, whole organism, appendix, bronchus, cartilage, heart, kidney, lung, lymph node, placenta, right cerebellum, skeletal muscle, testis, thymus. This information was derived by determining the tissue sources of the sequences that were included in the invention including but not limited to SeqCalling sources, Public EST sources, Literature sources, and/or RACE sources.

Possible small nucleotide polymorphisms (SNPs) found for NOV 14re listed in Table 14C.

TABLE 14C
SNPs

| | Nucleotide | Base | Amino Acid | |
|---|---|---|---|---|
| Variant | Position | Change | Position | Base Change |
| 13376532 | 92 | G > A | 30 | Gly > Asp |
| 13374885 | 262 | T > C | 87 | Ser > Pro |
| 13374884 | 296 | T > C | 98 | Leu > Pro |
| 13374883 | 385 | G > A | 128 | Val > Ile |
| 13376150 | 533 | A > G | 177 | Asn > Ser |
| 13376531 | 554 | T > C | 184 | Leu > Pro |
| 13376151 | 598 | G > A | 199 | Val > Met |
| 13376530 | 619 | G > A | 206 | Ala > Thr |
| 13376152 | 697 | A > G | 232 | Thr > Ala |

NOV14 also has homology to the amino acid sequences shown in the BLASTP data listed in Table 14D.

TABLE 14D
BLAST results for NOV14

| Gene Index/ | Protein/ | | Identity | Positives | |
|---|---|---|---|---|---|
| Identifier | Organism | Length (aa) | (%) | (%) | Expect |
| gi\|6136557\|sp\|07514 | HYPOTHETICAL | 558 | 420/500 | 420/500 | 0.0 |
| 4\|Y653_HUMAN | PROTEIN | | (84%) | (84%) | |
| | KIAA0653 | | | | |
| | PROTEIN | | | | |
| gi\|17450870\|ref\|XP_{—} | KIAA0653 | 302 | 259/284 | 259/284 | e−148 |
| 036027.2\| | protein, B7- | | (91%) | (91%) | |
| (XM_036027) | like protein | | | | |
| | [Homo sapiens] | | | | |
| gi\|6983944\|gb\|AAF34 | B7-like | 309 | 258/283 | 258/283 | e−147 |
| 739.1\|AF199028_1 | protein [Homo | | (91%) | (91%) | |
| (AF199028) | sapiens] | | | | |
| gi\|7657220\|ref\|NP_0 | icos ligand | 322 | 112/234 | 143/234 | 8e−50 |
| 5S605.1\| | [Mus musculus] | | (47%) | (60%) | |
| (NM_015790) | | | | | |
| gi\|15011418\|gb\|AAK7 | B7-like | 347 | 112/234 | 143/234 | 2e−49 |
| 7544.1\|AF394451_1 | protein GL50-B | | (47%) | (60%) | |
| (AF394451) | [Mus musculus] | | | | |

The homology of these sequences is shown graphically in the ClustalW analysis shown in Table 14E.

[image]

[image]

[image]

Table 14F lists the domain description from DOMAIN analysis results against NOV14. This indicates that the NOV14 sequence has properties similar to those of other proteins known to contain these domains.

TABLE 14F
Domain Analysis of NOV14

gnl|Smart|smart00406, UDPGT, IGv, Immunoglobulin V-Type (SEQ ID NQ:146)
Length = 80 residues, 100.0% aligned
Score = 38.9 bits (146), Expect = 7e-04

| | | | |
|---|---|---|---|
| Query: | 32 | DVELSCACPEGSRFDLNDVYVYWQTSESKTVVTYHIPQNSSLENVDSRYRNRALMSPAGM | 91 |
| | | \| \|\|\| + \| \| + \|\| \| + + \| + ++ \|+ \| +\| | |
| Sbjct: | 1 | SVTLSC---KASGFTFSSYYVSWVRQPPGKGLEWLGYIGSDVSYSEASYKGRVTISKD-N | 56 |
| Query: | 92 | LRGDFSLRLFNVTPQDEQKFHCLV 115 | |
| | | + \| \|\| + \|+ +\| ++\| \| | |
| Sbjct: | 57 | SKNDVSLTISNLRVEDTGTYYCAV 80 | |

Costimulatory interactions between the B7 family ligands and their receptors play critical roles in the growth, differentiation, and death of T cells. Engagement of the T-cell costimulator CD28 by either specific antibodies or its natural ligands B7-1 and B7-2 increases antigen-specific proliferation of CD4⁺ T cells, enhances production of cytokines, induces maturation of CD8⁺ effector T cells and promotes T-cell survival. Signaling through homologous CTLA-4 receptor of B7-1 and B7-2 on activated T cells, however, is thought to deliver a negative signal that inhibits T-cell proliferation, interleukin (IL)-2 production, and cell cycle progression. Although B7-1 and B7-2 share only ˜20% homology in their amino acids, they have similar tertiary structures and costimulatory functions.

Recent studies indicate that other members of the B7-CD28 family may also participate in the regulation of cellular and humoral immune responses. One of the new members is an inducible costimulator (ICOS), a CD28-like receptor. An F44 monoclonal antibody (mAb) against human ICOS costimulates T-cell growth and increases secretion of several cytokines including IL-10, interferon-, and IL4, but not IL-2 in the presence of optimal doses of anti-CD3 antibody. Another new B7 family member is mouse B7h/B7RP-1. B7h/B7RP-1 does not bind to CD28 and CTLA-4 and can costimulate T-cell growth in the presence of antigenic signals. It has been shown that surface expression of B7B7RP-1 is up-regulated by tumor necrosis factor-in the 3T3 fibroblast line and the increase of B7h/B7RP-1 messenger RNA (mRNA) is also observed in nonlymphoid tissues exposed to lipopolysaccharide (LPS). Yoshinaga and associates demonstrated that B7h/B7RP-1 is a ligand for mouse CRP-1, a mouse homologue of human ICOS. Expression of a B7RP-1 fusion protein in transgenic mice leads to hyperplasia in several lymphoid organs and treatment of mice with B7h/B7RP-1 fusion protein enhanced oxazolone-induced contact hypersensitivity.

A new member of the human B7 family, B7-H1, shares ˜20% identical amino acid sequence with B7-1 and B7-2 in the Ig V- and Ig C-like extracellular domains but differs more profoundly from B7-1 and B7-2 in the cytoplasmic domain. It is unlikely that B7-H1 is a human homologue of mouse B7h/B7RP-1 because identity of amino acids between them is less than 30%. Furthermore, B7-H1 does notbind to CD28, CTLA-4, and ICOS. Surface expression of B7-H1 can be detected in the majority of activated CD14+macrophages and a fraction of activated T cells. B7-H1 costimulates T-cell responses in the presence of suboptimal doses of anti-CD3 mAb, enhances allogeneic mixed lymphocyte response, and preferentiallyinduces IL-10 secretion from T cells. By searching molecules sharing homologies with the Ig V and Ig C domains of B7-1, B7-2, and B7-H1 in the NCBI database (http://www.ncbi.nlm.nih.gov) followed by subsequent cloning and sequencing, a new B7-like gene designated B7-H2 (B7 homologue 2) was identified. In addition to an overall structure similarity to B7-1, B7-2, and B7-H1, B7-H2 binds ICOS and costimulates the proliferation and cytokine production of human T cells. Cell surface expression of B7-H2 protein is detected in monocyte-derived immature dendritic cells. Soluble B7-H2 and immunoglobulin (Ig) fusion protein, B7-H2Ig, binds activated but not resting T cells and the binding is abrogated by inducible costimulator Ig (ICOSIg), but not CTLA4Ig. In addition, ICOSIg stains Chinese hamster ovary cells transfected with B7-H2 gene. By suboptimal cross-linking of CD3, costimulation of T-cell proliferation by B7-H2Ig is dose-dependent and correlates with secretion of interleukin (IL)-2, whereas optimal CD3 ligation preferentially stimulates IL-10 production. The results indicate that B7-H2 is a putative ligand for the ICOS T-cell molecule. (Blood. 2000;96:2808-2813) PMID: 11023515, UI: 20477846

The T cell-specific cell surface receptors CD28 (OMIM #186760) and CTLA4 (OMIM #123890) are important regulators of the immune system. CD28 potently enhances those T-cell functions essential for an effective antigen-specific immune response, and CTLA4 counterbalances the CD28-mediated signals and thus prevents an otherwise fatal overstimulation of the lymphoid system. By generating monoclonal antibodies against activated human T cells, Hutloff et al. (1999) identified another member of this family of molecules, ‘inducible costimulator,’ symbolized ICOS. The ICOS-specific monoclonal antibody did not react with resting human peripheral blood T cells, but stained CD4+ and CD8+ T lymphocytes that had been activated by stimulation of the T-cell antigen receptor complex. Immunoprecipitations defined the ICOS antigen as a disulfide-linked dimer with an apparent relative molecular mass of 55 to 60 kD. Protein purification by SDS-PAGE indicated that ICOS is expressed on the cell surface as a homodimeric protein, with the 2 chains differing only in their posttranslational modification. The full-length ICOS cDNA of 2,641 basepairs was cloned from a MOLT-4V T lymphoblast cDNA library. Northern analysis revealed a single ICOS mRNA species of approximately 2.8 kb in length in activated human T cells. The open reading frame of ICOS mRNA encodes a protein of 199 amino acids. The ICOS amino acid sequence shares 24% and 17% identity, respectively, with CD28 and CTLA4. The predicted mature ICOS is a type I transmembrane molecule that consists of a single immunoglobulin V-like domain, stabilized by conserved cysteine residues at positions 42 and 109; a transmembrane region of approximately 23 amino acids; and a cytoplasmic tail of 35 amino acids. It shows close structural resemblance to CD28 and CTLA4. The cysteine residue located at position 141 of CD28, also found in CTLA4, is apparently involved in forming the disulfide bridge between the homodimeric chains of these proteins, and is also found in ICOS at position 136. ICOS matches CD28 in potency and enhances all basic T-cell responses to a foreign antigen, namely proliferation, secretion of lymphokines, upregulation of molecules that mediate cell-cell interaction, and effective help for antibody secretion by B cells. Unlike the constitutively expressed CD28, ICOS has to be de novo induced on the T-cell surface and does not upregulate the production of interleukin-2 (IL2; OMIM #147680), but superinduces the synthesis of interleukin-10 (IL10; OMIM #124092), a B-cell differentiation factor. In vivo, ICOS is highly expressed on tonsillar T cells, which are closely associated with B cells in the apical light zone of germinal centers, the site of terminal B-cell maturation

Dong et al. (2001) generated Icos-deficient mice and determined that the absence of Icos did not impair T-cell development. However, T-cell activation in terms of proliferation and IL2 production was impaired. Differentiated Icos −/− cells were able to produce IFNG (OMIM #147570) but not IL4 (OMIM #147780) or IL2. In vivo immunization also revealed a defect in IL2 and IL4 production and a reduction in serum IgGI and IgE. Using allergy models, Dong et al. (2001) found that Icos was not required for Th2 cell differentiation, but rather it regulated IL4 and IL13 (OMIM #147683) production. Using the experimental autoimmune encephalitis (EAE) model for multiple sclerosis, the authors found that Icos −/−mice developed greatly enhanced disease compared with wildtype mice, even with a genetic background otherwise associated with resistance to EAE. Splenocytes from the knockout and wildtype mice produced undetectable levels of IL4 and similar levels of IL10 and IFNG; however, cells from the Icos −/− mice produced no IL13, whereas wildtype mice made abundant amounts. Dong et al. (2001) concluded that ICOS may have an important negative regulatory role, through the induction of IL13, in protection against inflammatory diseases.

McAdam et al. (2001) found that Icos-deficient mice had similar basal levels of IgM, slightly elevated IgG3, and reduced IgG1, IgG2a, and IgE compared to wildtype mice. Immunized knockout and wildtype mice, except in the presence of the highly inflammatory complete Freund's adjuvant, also had similar levels of IgM-specific antibody but reduced IgG1- and IgG2a-specific antibody and reduced germinal center formation. Class switching from IgM to IgG was restored in Icos −/−mice by stimulation of CD40 (OMIM #109535).

Tafuri et al. (2001) found that reduced T-cell proliferation in cells from Icos-deficient mice was associated with a marked decrease in expression of CD40LG (OMIM #308230), CD25 (IL2RA; OMIM #147730), and CD69 (OMIM #107273). B-cell activation and T cell-independent antibody responses were unimpaired in Icos knockout mice. In contrast to the findings of McAdam et al. (2001), Tafuri et al. (2001) found that only basal levels of IgGI were significantly reduced in Icos −/−mice; however, they concurred that serum IgG1 and IgG2a levels were reduced, and IgE levels were undetectable after immunization. ELISA assays showed that this class-switching impairment was associated with reduced IL4 production but not with IFNG production. Immunohistochemistry analysis determined that germinal center formation was also reduced in Icos knockout mice, as it is in mice deficient in Cd401g or Cd28.

The protein similarity information, expression pattern, and map location for the NOV14 protein and nucleic acid disclosed herein suggest that it may have important structural and/or physiological functions characteristic of the B7 Immunoglobulins family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: brain disorders including epilepsy, eating disorders, schizophrenia, ADD, and cancer; heart disease; inflammation and autoimmune disorders including Crohn's disease, IBD, allergies, rheumatoid and osteoarthritis, inflammatory skin disorders, allergies, blood disorders; psoriasis colon cancer, leukemia AIDS; thalamus disorders; metabolic disorders including diabetes and obesity; lung diseases such as asthma, emphysema, cystic fibrosis, and cancer; pancreatic disorders including pancreatic insufficiency and cancer; and prostate disorders including prostate cancer and other diseases, disorders and conditions of the like. These materials are further useful in the generation of antibodies that bind immunospecifically to the substances of the invention for use in therapeutic or diagnostic methods.

The B7-H2B-like nucleic acid and protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV14 protein has multiple hydrophilic regions, each of which can be used as an immunogen. In one embodiment, a contemplated NOV14 epitope is from about amino acids 20 to 25. In another embodiment, a contemplated NOV14 epitope is from about amino acids 48 to 49. In other specific embodiments, contemplated NOV10 epitopes are from about amino acids 50 to 52, 58 to 75, 100 to 120, 150 to 190, 240 to 260, 290 to 350, 370 to 420 and 440 to 450.

NOV15

A disclosed NOV15 nucleic acid(designated CuraGen Acc. No. CG56252-01) encoding a novel galactosyltransferase-like protein includes 1302 nucleotides (SEQ ID NO: 49) and is shown in Table 15A. An open reading frame was identified beginning with an ATG initiation codon at nucleotides 1-3 and ending with a TAG codon at nucleotides 1276-1278. A putative untranslated region downstream from the termination codon is underlined in Table 15A, and the start and stop codons are in bold letters.

TABLE 15A
NOV15 Nucleotide Sequence

(SEQ ID NO:49)

ATGGGAGGAGCTGCCCCCTGCTGCCGACTGCGACCCTTACAGGGGAGGGAGGGCGCAGGCCGCGCGGAGA
TGAGGAGGAGGCTGCGCCTACGCAGGGACGCATTGCTCACGCTGCTCCTTGGCGCCTCCCTGGGCCTCTT
ACTCTATGCGCAGCGCGACGGCGCGGCCCCGACGGCGAGCGCGCCGCGAGGGCGAGGGAGGGCGGCACCG
AGGCCCACCCCCGGACCCCGCGCGTTCCAGTTACCCGACGCGGGTGCAGCCCCGCCGGCCTACGAAGGGG
ACACACCGGCGCCGCCCACGCCTACGGGACCCTTTGACTTCGCCCGCTATTTGCGCGCCAAGGACCAGCG
GCGGTTTCCACTGCTCATTAACCAGCCGCACAAGTGCCGCGGCGACGGCGCACCCGGTGGCCGCCCGGAC
CTGCTTATTGCTGTCAAGTCGGTGGCAGAGGACTTCGAGCGGCGCCAAGCCGTGCGCCAGACGTGGGGCG
CGGAGGGTCGCGTGCAGGGGGCGCTGGTGCGCCGCGTGTTCTTGCTGGGCGTGCCCAGGGGCGCAGGCTC
GGGCGGGGCCGACGAAGTTGGGGAGGGCGCGCGAACCCACTGGCGCGCCCTGCTGCGGGCCGAGAGCCTT
GCGTATGCGGACATCCTGCTCTGGGCCTTCGACGACACCTTTTTTAACCTAACGCTCAAGGAGATCCACT
TTCTAGCCTGGGCCTCAGCTTTCTGCCCCGACGTGCGCTTCGTTTTTAAGGGCGACGCAGATGTGTTCGT
GAACGTGGGAAATCTCCTGGAGTTCCTGGCGCCGCGGGACCCGGCGCAAGACCTGCTTGCTGGTGACGTA
ATTGTGCATGCGCGGCCCATCCGCACGCGGGCTAGCAAGTACTACATCCCCGAGGCCGTGTACGGCCTGC
CCGCCTATCCGGCCTACGCGGGCGGCGGTGGCTTTGTGCTTTCCGGGGCCACGCTGCACCGCCTGGCTGG
CGCCTGTGCGCAGGTCGAGCTCTTCCCCATCGACGACGTCTTTCTGGGCATGTGTCTGCAGCGCCTGCGG
CTCACGCCCGAGCCTCACCCTGCCTTCCGCACCTTTGGCATCCCCCAGCCTTCAGCCGCGCCGCATTTGA
GCACCTTCGACCCCTGCTTTTACCGTGAGCTGGTTGTAGTGCACGGGCTCTCGGCCGCTGACATCTGGCT
TATGTGGCGCCTGCTGCACGGGCCGCATGGGCCAGCCTGTGCGCATCCACAGCCTGTCGCTGCAGGCCCC
TTCCAATGGGACTCCTAGCTCCCCACTACAGCCCCAAGCTCC

The nucleic acid sequence of NOV15 maps to chromosome 16 and has 421 of 639 bases (65%) identical to a gb:GENBANK-ID:AF175522|acc:AF175522.1 mRNA from Homo sapiens (Homo sapiens transmembrane tryptase mRNA, complete cds) (E 1.9e⁻³³).

A NOV15 polypeptide (SEQ ID NO: 50) is 425 amino acid residues and is presented using the one letter code in Table 13B. Signal P, Psort and/or Hydropathy results predict that NOV15 contains a signal peptide and is likely to be localized at the plasma membrane with a certainty of 0.7900. In other embodiments, NOV15 is localized to the microbody (peroxisome) with a certainty of 0.6400, the Golgi body with a certainty of 0.3000 or the endoplasmic reticulum (membrane) with a certainty of 0.2000. The most likely cleavage site for a NOV15 peptide is between amino acids 55 and 56, at: DGA-AP.

TABLE 15B
NOV15 protein sequence

(SEQ ID NO:50)

MGGAAPCCRLRPLQGREGAGRAEMRRRLRLRRDALLTLLLGASLGLLLYAQRDGAAPTASAPRGRGRAAPRPTP
GPRAFQLPDAGAAPPAYEGDTPAPPTPTGPFDFARYLRAKDQRRFPLLINQPHKCRGDGAPGGRPDLLIAVKSV
AEDFERRQAVRQTWGAEGRVQGALVRRVFLLGVPRGAGSGGADEVGEGARTHWRALLRAESLAYADILLWAFDD
TFFNLTLKEIHFLAWASAFCPDVRFVFKGDADVFVNVGNLLEFLAPRDPAQDLLAGDVIVHARPIRTRASKYYI
PEAVYGLPAYPAYAGGGGFVLSGATLHRLAGACAQVELFPIDDVFLGMCLQRLRLTPEPHPAFRTFGIPQPSAA
PHLSTFDPCFYRELVVVHGLSAADIWLMWRLLHGPHGPACAHPQPVAAGPFQWDS

The NOV15 amino acid sequence has 93 of 201 amino acid residues (460%) identical to, and 125 of 201 amino acid residues (62%) similar to, the 342 amino acid residue ptnr:TREMBLNEW-ACC:AAG32641 protein from Rattus norvegicus (Rat) (PROSTASIN) (E=9.6e⁻⁵⁵).

NOV15 is expressed in at least the following tissues: large cell carcinoma, adult brain, amygdala, aorta, appendix, artery, bone marrow, brain, cartilage, cerebellum, cervix, epidermis, kidney, lung, lymph node, lymphoid tissue, ovary, oviduct/uterine tube/fallopian tube, pituitary gland, prostate, skin, small intestine, spinal cord, spleen, synovium/synovial membrane, testis, thalamus, thymus, thyroid, vulva, whole organism, bone marrow. This information was derived by determining the tissue sources of the sequences that were included in the invention including but not limited to SeqCalling sources, Public EST sources, Literature sources, and/or RACE sources. In addition, the sequence is predicted to be expressed in the following tissues because of the expression pattern of (GENBANK-ID: gb:GENBANK-ID:AB015630|acc:AB015630.1) a closely related Homo sapiens mRNA for type II membrane protein, complete cds, clone:HP10328 homolog in species Homo sapiens :epidermoid carcinoma.

NOV15 also has homology to the amino acid sequences shown in the BLASTP data listed in Table 15C.

TABLE 15C
BLAST results for NOV15

| Gene Index/ | Protein/ | | | | |
|---|---|---|---|---|---|
| Identifier | Organism | Length (aa) | Identity {%) | Positives (%) | Expect |
| gi\|15208631\|ref\|NP_{—} | hypothetical | 377 | 303/317 | 303/317 | e−163 |
| 171608.1\| | protein MGC4655 | | (95%) | (95%) | |
| (NM_033309) | [Homo sapiens] | | | | |
| gi\|17225044\|gb\|AAL3 | beta-1,3- | 399 | 272/347 | 284/347 | e−141 |
| 7220.1\|AF321826_1 | galactosyltrans- | | (78%) | (81%) | |
| (AF321826) | ferase-related | | | | |
| | protein [Mus | | | | |
| | musculus] | | | | |
| gi\|16973455\|gb\|AAL3 | beta-3- | 418 | 128/295 | 181/295 | 1e−65 |
| 2295.1\|AF321827_1 | galactosyltrans- | | (43%) | (60%) | |
| (AF321827) | ferase [Danio | | | | |
| | rerio] | | | | |
| gi\|16973459\|gb\|AAL3 | beta-3- | 412 | 123/289 | 171/289 | 2e−58 |
| 2297.1\|AF321829_1 | galactosyltrans- | | (42%) | (58%) | |
| (AF321829) | ferase [Danio | | | | |
| | rerio] | | | | |
| gi\|14290592\|gb\|AAH0 | beta-1,3-N- | 397 | 124/299 | 163/299 | 7e−53 |
| 9075.1\|AAH09075 | acetylglucosamin | | (41%) | (54%) | |
| (BC009075) | yltransferase 1 | | | | |
| | [Mus musculus] | | | | |

The homology of these sequences is shown graphically in the ClustalW analysis shown in Table 15D.

[image]

[image]

Table 15E lists the domain description from DOMAIN analysis results against NOV15. This indicates that the NOV15 sequence has properties similar to those of other proteins known to contain these domains.

TABLE 15E
Domain Analysis of NOV15

gnl|Pfam|pfam01762, Galactosyl_T, Galactosyltransferase
(SEQ ID NO:152)
Length = 195sidues, 99.5igned
Score = 102 bits (254), Expect = 4e−23

| | | | |
|---|---|---|---|
| Query: | 154 | RRQAVRQTWGAEGRVQGALVRRVFLLGVPRGAGSGGADE-VGEGARTHWRALLRAESLAY | 212 |
| | | \|\| \|+\|+\|\| + +\| ++ +\|\|+\| \| \| + \| \| \|\| \| | |
| Sbjct: | 2 | RRNAIRKTWMNQNNSRGGRIKSLFLVG--LAALDGKLKKLVMEEARL------------Y | 47 |
| Query: | 213 | ADILLWAFDDTFFNLTLKEIHFLAWASAFCPDVRFVFKGDADVFVNVGNLLEFL--APRD | 270 |
| | | \|\|++ +\|++ +\|\|\|\|\| + \| + + \|\|+ + + \| \| \|\|\|\|\| \|\|\| \| \| | |
| Sbjct: | 48 | GDIIVVDLEDSYLNLTLKTLTILLYVVSKCPNAKLIGKIDDDVFVNPDNLLSLLEREYID | 107 |
| Query: | 271 | PAQDLLAGDVIVHARPIRTRASKYYIPEAVYGLPAYPAYAGGGGFVLSGATLHRLAGACA | 330 |
| | | \|+ \| +\| + \|+\|\|+ \|\|+\|+\| \| \|\| \| \| ++\|\| + \| | |
| Sbjct: | 108 | PSPLSFYGYIIKNGEPVRTKKSKWYVPPTAYPCSNYPPYLSGPFYILSRDAAPLILKASK | 167 |
| Query: | 331 | QVELFPIDDVFL-GMCLQRLRLTPEPHP 357 | |
| | | \|+\|\| + \|+ \| ++ \| | |
| Sbjct: | 168 | HRRFIKIEDVLITGILALDLGISRINLP 195 | |

The enzyme galactosyltransferase (EC 2.4.1.38) catalyzes the reaction involving UDP-galactose and N-acetylglucosamine for the production of galactose beta-1,4-N-acetylglucosamine. The enzyme that provides UDP-galactose for galactosyltransferase, galactose-1-phosphate uridyltransferase, maps to the same band. The galactosyltransferase enzyme can also form a heterodimer with the regulatory protein alpha-lactalbumin to form lactose synthetase (EC 2.4.1.22). In addition to a biosynthetic role, galactosyltransferases may be components of plasma membranes where they may function in intercellular recognition and/or adhesion. Masri et al. (1988) noted that galactosyltransferase, which they called beta-1,4-galactosyltransferase, is located primarily in the trans-cistemae of the Golgi complex and exists in both membrane-bound and soluble forms.

Appert et al. (1986) cloned a galactosyltransferase cDNA by screening a human liver cDNA library with a probe based on the sequence of the purified protein. The partial cDNA did not include the putative N-terminal membrane-bound region. By screening a human placenta cDNA library with the partial galactosyltransferase cDNA isolated by Appert et al. (1986), Masri et al. (1988) cloned a full-length beta-1,4-galactosyltransferase cDNA. It encodes a predicted 400-amino acid protein with an N-terminal membrane-anchoring domain. The soluble form of the enzyme appears to result from proteolytic cleavage of the membrane-bound form at arginine-77. Mengle-Gaw et al. (1991) reported that the galactosyltransferase gene, which they called GalTase, is composed of 6 exons spanning more than 50 kb. By Northern blot analysis, GalTase was expressed as a 4.2-kb mRNA in all cell lines examined; there was a high degree of variability in expression levels among the cell lines. Appert et al. (1986) mapped the galactosyltransferase gene to chromosome 9. Shaper et al. (1986) localized the structural gene for galactosyltransferase to 9p 13 by in situ hybridization using a cloned bovine cDNA probe. On the basis of dosage effects, Furukawa et al. (1986) suggested that several galactosyltransferase genes may be located on chromosome 17 of the mouse; trisomy 17 embryos had enzyme activities almost 1.5 times higher than did diploid embryos. Furukawa et al. (1986) suggested a relationship between these galactosyltransferases and the major histocompatibility complex. Lo et al. (1998) analyzed 6 members of the B4GALT galactosyltransferase family. Northern blot analysis revealed that, among these homologs, only B4GALT1 is expressed in the mouse lactating mammary gland. They stated that B4GALT1 null mice are unable to produce lactose. Thus, B4GALT1 appears to be the gene recruited for lactose biosynthesis during the evolution of mammals.

The protein similarity information, expression pattern, and map location for the NOV15 protein and nucleic acid disclosed herein suggest that this it may have important structural and/or physiological functions characteristic of the Galactosyltransferase family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: proteodermatan sulfate, defective biosynthesis of PDS, defective biosynthesis of dermatan sulfate proteoglycan xylosylprotein 4-beta-galactosyltransferase deficiency, xgpt deficiency galactosyltransferase I deficiency, Ehlers-danlos syndrome, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, endometriosis, fertility, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection and other diseases, disorders and conditions of the like.

The novel nucleic acid encoding the galactosyltransferase-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV10 protein has multiple hydrophilic regions, each of which can be used as an immunogen. In one embodiment, a contemplated NOV15 epitope is from about amino acids 30 to 45. In another embodiment, a contemplated NOV15 epitope is from about amino acids 60 to 65. In other specific embodiments, contemplated NOV15 epitopes are from about amino acids 80 to 110, 140 to 145, 155 to 165, 170 to 175, 180 to 183, 190 to 192 and 210 to 260.

NOV16

A disclosed NOV16 nucleic acid (designated CuraGen Acc. No. CG56303-01) encoding a novel lymphocyte antigen precursor-like protein includes 447 nucleotides (SEQ ID NO: 51) and is shown in Table 16A. An open reading frame was identified beginning with an ATG initiation codon at nucleotides 31-33 and ending with a TGA codon at nucleotides 418-420. A putative untranslated region downstream from the termination codon is underlined in Table 13A, and the start and stop codons are in bold letters.

TABLE 16A
NOV16 Nucleotide Sequence

(SEQ ID NO:51)

GGCCCGCACTGCTCCCAGACGACATCAGAGATGAGGACAGCATTGCTGCTCCTTGCAGCCCTGGCTGTGG
CTACAGGGCCAGCCCAGGCCTTGGACTGCCACGTGTGTGCCTACAACGGAGACAACTGCTTCAACCCCAT
GCGCTGCCCGGCTATGGTTGCCTACTGCATGACCACGCGCACCTGTGAGCCACTTCGCGGGAGAGAACTT
AAGAAGGACTGTGCGAAGTGGTGCACACCCGGTTACCCCTTGCAAGGCCAGGTCAGCAGCGGCACAGCTT
CCACCCAGTGCTGCAGGGAGGACCTGTGCAATGAGAAGCTGCACAACGCTGCACCCACCCGCACCGCCCT
CGCCCACAGTGCCCTCAGCCTGGGGCTGGCCCTGAGCCTCCTGGCCGTCATCTTAGCCCCCAGCCTGTGA
CCTTCCCCGCAGGGAAGGCCCCTCATG

The nucleic acid sequence of NOV16 maps to chromosome 8 has 383 of 440 bases (87%) identical to a gb:GENBANK-ID:A58084| acc:A58084 mRNA from Homo sapiens (Sequence 1 from Patent WO9635808 (E=3.6e⁻⁶⁷).

A NOV16 polypeptide (SEQ ID NO: 51) 129 amino acid residues and is presented using the one letter code in Table 16B. Signal P, Psort and/or Hydropathy results predict that NOV16 contains a signal peptide and is likely to be localized at the plasma membrane with a certainty of 0.9190. In other embodiments, NOV16 is localized to the lysosome (membrane) with a certainty of 0.2000, the endoplasmic reticulum (membrane) with a certainty of 0.1000 or the endoplasmic reticulum (lumen) with a certainty of 0.1000. The most likely cleavage site for a NOV16 peptide is between amino acids 20 and 21, at: AQA-LD.

TABLE 16B
NOV16 protein sequence

(SEQ ID NO:52)

MRTALLLLAALAVATGPAQALDCHVCAYNGDNCFNPMRCPAMVAYCMTTRTCEPLRGRELKKDCAKWCTPGYPL
QGQVSSGTASTQCCREDLCNEKLHNAAPTRTALAHSALSLGLALSLLAVILAPSL

The NOV16 amino acid sequence has 100 of 129 amino acid residues (77%) identical to, and 108 of 129 amino acid residues (83%) similar to, the 128 amino acid residue ptnr:SWISSNEW-ACC:Q14210 protein from Homo sapiens (Human) (LYMPHOCYTE ANTIGEN LY-6D PRECURSOR (E48 ANTIGEN)(E=5.1e⁻⁴⁸).

NOV16 is predicted to be expressed in at least the following tissues: Human Breast Adenocarcinoma.

Possible small nucleotide polymorphisms (SNPs) found for NOV16 are listed in Table 16C.

TABLE 16C
SNPs

| | Nucleotide | | Amino Acid | |
|---|---|---|---|---|
| Variant | Position | Base Change | Position | Base Change |
| C99.827 | 1793 | G > A | 598 | Arg > Gln |

NOV16 also has homology to the amino acid sequences shown in the BLASTP data listed in Table 16D.

TABLE 16D
BLAST results for NOV16

| Gene Index/ | Protein/ | | | | |
|---|---|---|---|---|---|
| Identifier | Organism | Length (aa) | Identity (%) | Positives (%) | Expect |
| gi\|11321575\|ref\|N | lymphocyte | 128 | 60/105 | 68/105 | 1e−18 |
| P_003686.1\| | antigen 6 | | (57%) | (64%) | |
| (NM_003695) | complex, locus | | | | |
| | D; e48 antigen | | | | |
| | [Homo sapiens] | | | | |
| gi\|2739294\|emb\|CA | E48 antigen | 128 | 60/105 | 68/105 | 3e−18 |
| A73189.1\| | [Homo sapiens] | | (57%) | (64%) | |
| (Y12642) | | | | | |
| gi\|2118925\|pir\|\|I | gene ThB | 130 | 42/80 | 52/80 | 4e−13 |
| _54553 | protein-mouse | | (52%) | (64%) | |
| gi\|6754584\|ref\|NP | lymphocyte | 127 | 42/80 | 52/80 | 7e−13 |
| _034872.1\| | antigen 6 | | (52%) | (64%) | |
| (NM_010742) | complex, locus D | | | | |
| | [Mus musculus] | | | | |
| gi\|1519481\|gb\|AAB | E48 antigen | 79 | 47/75 | 56/75 | 8e−13 |
| 07524.1\| (U66837) | [Homo sapiens] | | (62%) | (74%) | |

The homology of these sequences is shown graphically in the ClustalW analysis shown in Table 16E.

[image]

[image]

Table 16F lists the domain description from DOMAIN analysis results against NOV16. This indicates that the NOV16 sequence has properties similar to those of other proteins known to contain these domains.

TABLE 16F
Domain Analysis of NOV16

gnl|Smart|smart00134, LU, Ly-6 antigen/uPA receptor-like domain
(SEQ ID NO:158)
Length = 91 residues, 96.7% aligned
Score = 43.9 bits (102), Expect = 6e−06

| | | | |
|---|---|---|---|
| Query: | 22 | DCHVCAYNGDNCFNPMRCPAMVAYCMTTRTCEPLRGRE--LKKDCAK--WCTPGYPLQGQ | 77 |
| | | C+ C N D+ + C + C+T R + K CA C + ++ | |
| Sbjct: | 2 | QCYSCTGNPDSSCSTEECRSPDDVCLTAVAEVISGSRGSVVYKGCATSPICPGSHGIEIH | 61 |
| Query: | 78 | VSSGTASTQCCREDLCNEKLHNAAPTRT 105 | |
| | | ++ S CC+ DLCN T T | |
| Sbjct: | 62 | LTIANVSVSCCQTDLCNAAGPTLGSTLT 89 | |

Ly-6A.2 and Ly-6E.1 molecules are antithetical and identical to MALA-1. On western blots of lymphocyte surface proteins which had been solubilized and electrophoretically separated in octylglucoside, bands were detected which comigrated with Ly-6A.2 or Ly-6E.1 antigens. On cells or in an immunoassay they blocked alloantibodies against Ly-6A.2 or Ly-6E.1. The tissue distribution of MALA-1 also correlated with Ly-6A.2 or Ly-6E.1. Upon octylglucoside or sodium dodecyl sulfate-polyacrylamide gel electrophoresis, these antigens displayed similar sizes. Thus, Ly-6A.2 and Ly-6E.1 are most likely products of alternate alleles. Electrophoretic analysis showed a similar size and charge for Ly-6A.2, Ly-6B.2, Ly-6D.2, and Ly-27.2. Ly-6C.2 and Ly-28.2 appeared to be identical, and were similar in size to Ly-6A.2, but they differed in charge and in intrachain disulfide constraints. Since Ly-6D.2 and Ly-27.2 may represent the same or different epitopes on the Ly-6A.2 molecule, the previously postulated five Ly-6-like antigens that were thought to be separable on the basis of tissue distribution, may represent no more than three separate proteins which can be assigned to one of two distinct categories by electrophoretic mobility in gels containing octylglucoside.

Competitive binding studies and immunoprecipitation experiments define at least five distinct epitopes encoded by Ly-6-linked genes—Ly-6A.2, Ly-6B.2, Ly-6C.2, Ly-6D.2, and ThB. Ly-6A.2, a 33 kd protein, and Ly-6D.2 are closely overlapping epitopes that can be distinguished by their unique thymus reactions of 10-20% or greater than 90%, respectively. Similarly, the Ly-6C.2 antigen present on a 14 kd moiety loosely overlaps the Ly-6B.2 antigen. Ly-6C.2 and Ly-6B.2 antigens are distinct from Ly-6A.2 and Ly-6D.2, however. ThB is a 16-18 kd antigen which is not associated on the cell surface with any other “Ly-6” antigens. In addition, independently derived antibodies made to the Ly-6C.2 antigen detect an identical epitope, as do antibodies to Ly-6A.2 and Ly-6B.2. These results imply the existence of a single antigenic site on each of these molecules.

Despite the differences in the antigens that they recognize and in the effector functions they carry out, B and T lymphocytes utilize remarkably similar signal transduction components to initiate responses. They both use oligomeric receptors that contain distinct recognition and signal transduction subunits. Antigen receptors on both cells interact with at least two distinct families of PTKs via common sequence motifs, ARAMs, in the cytoplasmic tails of their invariant chains, which have likely evolved from a common evolutionary precursor. Coreceptors appear to serve to increase the sensitivity of both of these receptor systems through events that influence ligand binding and signal transduction. The critical role of tyrosine phosphorylation of downstream signaling components, such as phospholipase C, is the net result of changes in the balance of the action of antigen receptor-regulated PTKs and PTPases. The identification of downstream effectors, including calcineurin and Ras, that regulate cellular responses, such as lymphokine gene expression, promises the future possibility of connecting the complex pathway from the plasma membrane to the nucleus in lymphocytes. Insight gained from studies of the signaling pathways downstream of TCR and BCR stimulation is likely to contribute significantly to future understanding of mechanisms responsible for lymphocyte differentiation and for the discrimination of self from nonself in developing and mature cells.

The E48 antigen, a putative human homologue of the 20-kD protein present in desmosomal preparations of bovine muzzle, and formerly called desmoglein III (dg4), is a promising target antigen for antibody-based therapy of squamous cell carcinoma in man. To anticipate the effect of high antibody dose treatment, and to evaluate the possible biological involvement of the antigen in carcinogenesis, we set out to molecularly characterize the antigen. A cDNA clone encoding the E48 antigen was isolated by expression cloning in COS cells. Sequence analysis revealed that the clone contained an open reading frame of 128 amino acids, encoding a core protein of 13,286 kD. Database searching showed that the E48 antigen has a high level of sequence similarity with the mouse ThB antigen, a member of the Ly-6 antigen family. Phosphatidylinositol-specific (PI-specific) phospholipase-C treatment indicated that the E48 antigen is glycosylphosphatidylinositol-anchored (GPI-anchored) to the plasma membrane. The gene encoding the E48 antigen is a single copy gene, located on human chromosome 8 in the 8q24-qter region. The expression of the gene is confined to keratinocytes and squamous tumor cells. The putative mouse homologue, the ThB antigen, originally identified as an antigen on cells of the lymphocyte lineage, was shown to be highly expressed in squamous mouse epithelia. Moreover, the ThB expression level is in keratinocytes, in contrast to that in lymphocytes, not mouse strain related. Transfection of mouse SV40-polyoma transformed mouse NIH/3T3 cells with the E48 cDNA confirmed that the antigen is likely to be involved in cell-cell adhesion.

The Thb locus is responsible for the expression of 15-kDa phosphatidyl inositol anchored molecules (ThB) on murine thymocytes and B cells. Thb expression as detected with mAb is polymorphic on B cells with two alleles, Thbh and Thbl responsible for the high and low expression of ThB on B cells. The regulatory locus for Thb expression had been mapped with the Ly-6 cluster of genes to Chr 15. In our study we used expression cloning in COS cells to isolate cDNA clones that code for ThB after transfection; the cDNA products react with anti- ThB antibodies, but not with Ly-6A.2, -6B.2, -6C.2, or -6D.2 antibodies. One of these clones, pThB-A contains insert of 702 bases which was sequenced. The translated amino acid sequence has 11 cysteine residues, and together with the absence of potential N-linked glycosylation sites is similar to the structure of the Ly-6 molecules. The nucleotide and amino acid sequences of ThB cDNA were compared to those of Ly-6 genes and the Ly-6 related human CD59 and show clear homology. Finally using interspecies crosses, the structural Thb gene has been mapped to Chr 15; thus both structural and regulatory genes map to a similar site. The genetic map location near Ly-6 and the sequence similarity suggest that Thb and Ly-6 may have been derived from the same progenitor by gene duplication The protein similarity information, expression pattern, and map location for the NOV16 protein and nucleic acid disclosed herein suggest that it may have important structural and/or physiological functions characteristic of the TGF family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from cancer,trauma, regeneration, viral/bacterial/parasitic infections.

The novel nucleic acid encoding the lymphocytic antigen precursor-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV16 protein has multiple hydrophilic regions, each of which can be used as an immunogen. In one embodiment, a contemplated NOV16 epitope is from about amino acids 25 to 30. In another embodiment, a contemplated NOV16 epitope is from about amino acids 50 to 70. In other specific embodiments, contemplated NOV10 epitopes are from about amino acids 82 to 102.

NOV17

A disclosed NOV17 nucleic acid (designated CuraGen Acc. No. CG56307-01) encoding a novel pepsinogen C-like protein includes 1270 nucleotides (SEQ ID NO: 53) and is shown in Table 13A. An open reading frame was identified beginning with an ATG initiation codon at nucleotides 8-10 and ending with a TAG codon at nucleotides 1124-1126. A putative untranslated region downstream from the termination codon is underlined in Table 17A, and the start and stop codons are in bold letters.

TABLE 17A
NOV17 Nucleotide Sequence

| | |
|---|---|
| CAGCATCATGAAGTGGATGGTGGTGGTCTTGGTCTGCCTCCAGCTCTTGGAGGCAGCAGTGGT | (SEQ ID NO:53) |
| CAAAGTGCCCCTGAAGAAATTTAAGTCTATCCGTGAGACCATGAAGGAGAAGGGCTTGCTGGG | |
| GGAGTTCCTGAGGACCCACAAGTATGATCCTGCTTGGAAGTACCGCTTTGGTGACCTCAGCGT | |
| GACCTACGAGCCCATGGCCTACATGGATGCTGCCTACTTTGGTGAGATCAGCATCGGGACTCC | |
| ACCCCAGAACTTCCTGGTCCTTTTTGACACCGGCTCCTCCAACTTGTGGGTGCCCTCTGTCTA | |
| CTGCCAGAGCCAGGCCTGCACCAGTCACTCCCGCTTCAACCCCAGCGAGTCGTCCACCTACTC | |
| CACCAATGGGCAGACCTTCTCCCTGCAGTATGGCAGTGGCAGCCTCACCGGCTTCTTTGGCTA | |
| TGACACCCTGACTGTCCAGAGCATCCAGGTCCCCAACCAGGAGTTCGGCTTGAGTGAGAATGA | |
| GCCTGGTACCAACTTCGTCTATGCGCAGTTTGATGGCATCATGGGCCTGGCCTACCCTGCTCT | |
| GTCCGTGGATGAGGCCACCACAGCTATGCAGGGCATGGTGCAGGAGGGCGCCCTCACCAGCCC | |
| CGTCTTCAGCGTCTACCTCAGCAACCAGCAGGGCTCCAGCGGGGGAGCGGTTGTCTTTGGGGG | |
| TGTGGATAGCAGCCTGTACACGGGGCAGATCTACTGGGCGCCTGTCACCCAGGAACTCTACTG | |
| GCAGATTGGCATTGAAGAGTTCCTCATCGGCGGCCAGGCCTCCGGCTGGTGTTCTGAGGGTTG | |
| CCAGGCCATCGTGGACACAGGCACCTCTCTGCTCACTGTGCCCCAGCAGTACATGAGTGCTCT | |
| TCTGCAGGCCACAGGGGCCCAGGAGGATGAGTATGGACAGTTTCTCGTGAACTGTAACAGCAT | |
| TCAGAATCTGCCCAGCTTGACCTTCATCATCAATGGTGTGGAGTTCCCTCTGCCACCTTCCTC | |
| CTATATCCTCAGTAACAACGGCCAGCCCCTGTGGATCCTCGGGGATGTCTTCCTCAGGTCCTA | |
| CTATTCCGTCTACGACTTGGGCAACAACAGAGTAGGCTTTGCCACTGCCGCCTAGACTTGCTG | |
| CCTCGACACGTGGGTGGGCTCCCCTCTTCCTCTTGACCCTGCACCCTCCTAGGGCATTGTATC | |
| TGTCTTTCCACTCTGGATTCAGCCTTCTTTTTCTGGACTCTGGACTTTCTCTAATAATAAATA | |
| GTTCTTCTTT | |

The nucleic acid sequence of NOV17 maps to chromosome 16q21.3-p21.1 and invention has 1171 of 1277 bases (91%) identical to a gb:GENBANK-ID:HUMPGCA|acc:J04443.1 mRNA from Homo sapiens (Homo sapiens pepsinogen C (PGC) mRNA, complete cds) (E=7.0e⁻²²⁸).

A NOV17 polypeptide (SEQ ID NO: 54) is 372 amino acid residues and is presented using the one letter code in Table 17B. Signal P, Psort and/or Hydropathy results predict that NOV17 contains a signal peptide and is likely to be localized at the outside of the cell with a certainty of 0.8200. In other embodiments, NOV17 is localized to the microbody (peroxisome) with a certainty of 0.2076, the endoplasmic reticulum (membrane) with a certainty of 0.1000 or the endoplasmic reticulum (lumen) with a certainty of 0.1000. The most likely cleavage site for a NOV17 peptide is between amino acids 16 and 17, at: LEA-AV.

TABLE 17B
NOV17 protein sequence

(SEQ ID NO:54)

MKWMVVVLVCLQLLEAAVVKVPLKKFKSIRETMKEKGLLGEFLRTHKYDPAWKYRFGDLSVTYEPMAYMDAAYF
GEISIGTPPQNFLVLFDTGSSNLWVPSVYCQSQACTSHSRFNPSESSTYSTNGQTFSLQYGSGSLTGFFGYDTL
TVQSIQVPNQEFGLSENEPGTNFVYAQFDGIMGLAYPALSVDEATTAMQGMVQEGALTSPVFSVYLSNQQGSSG
GAVVFGGVDSSLYTGQIYWAPVTQELYWQIGIEEFLIGGQASGWCSEGCQAIVDTGTSLLTVPQQYMSALLQAT
GAQEDEYGQFLVNCNSIQNLPSLTFIINGVEFPLPPSSYILSNNGQPLWILGDVFLRSYYSVYDLGNNRVGFAT
AA

The NOV17 amino acid sequence have 372 of 388 amino acid residues (95%) identical to, and 372 of 388 amino acid residues (95%) similar to, the 388 amino acid residue ptnr:SWISSPROT-ACC:P20142 protein from Homo sapiens (Human) (GASTRICSIN PRECURSOR (EC 3.4.23.3) (PEPSINOGEN C))(FIG. 3B). The sequence of this invention lacks 16 internal amino acids when compared to ptnr:SWISSPROT-ACC:P20142 protein from Homo sapiens (Human) (GASTRICSIN PRECURSOR (EC 3.4.23.3) (PEPSINOGEN C)) (E=1.1e⁻¹⁹⁷).

NOV17 is expressed in at least the following tissues: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus, aorta, duodenum, gall bladder, liver, lung, lung pleura, lymph node, ovary, peripheral blood. This information was derived by determining the tissue sources of the sequences that were included in the invention including but not limited to SeqCalling sources, Public EST sources, Literature sources, and/or RACE sources. In addition, the sequence is predicted to be expressed in gastric mucosa because of the expression pattern of (GENBANK-ID: gb:GENBANK-ID:HUMPGCA|acc:J04443.1) a closely related Homo sapiens pepsinogen C (PGC) mRNA, complete cds homolog in species Homo sapiens.

NOV17 also has homology to the amino acid sequences shown in the BLASTP data listed in Table 17C.

TABLE 17C
BLAST results for NOV17

| Gene Index/ | | | | | |
|---|---|---|---|---|---|
| Identifier | Protein/Organism | Length (aa) | Identity (%) | Positives (%) | Expect |
| gi\|4505757\|ref\|NP_0 | progastrics in | 388 | 357/388 | 357/388 | 0.0 |
| 02621.1\| | (pepsinogen C); | | (92%) | (92%) | |
| (NM_002630) | Preprogastricsin | | | | |
| | [Homo sapiens] | | | | |
| gi\|387014\|gb\| AAA600 | pepsinogen [Homo | 385 | 354/385 | 354/385 | 0.0 |
| 62.1\| (M18667) | sapiens] | | (91%) | (91%) | |
| gi\|129797\|sp\|P03955 | PROGASTRICSIN | 377 | 337/377 | 342/377 | 0.0 |
| \|PEPC_MACFU | PRECURSOR | | (89%) | (90%) | |
| | (PEPSINOGEN C) | | | | |
| gi\|11265695\|pir\|\|JC | pepsinogen C - | 388 | 333/388 | 344/388 | 0.0 |
| 7245 | common marmoset | | (85%) | (87%) | |
| gi\|9798666\|dbj\|BAB1 | pepsinogen C | 389 | 322/389 | 341/389 | e−171 |
| 1755.1\| (AB047249) | [Rhinolophus | | (82%) | (86%) | |
| | ferrumequinum] | | | | |

The homology of these sequences is shown graphically in the ClustalW analysis shown in Table 17D.

[image]

[image]

Table 17E lists the domain description from DOMAIN analysis results against NOV17. This indicates that the NOV17 sequence hasproperties similar to those of other proteins known to contain these domains.

TABLE 17E
Domain Analysis of NOV17

gnl|Pfam|pfam00026, asp, Eukaryotic aspartyl protease SEQ ID NO:164)
Length = 376 residues, 100.0% aligned
Score = 402 bits (1034), Expect = 1e−113

| | | | |
|---|---|---|---|
| Query: | 18 | VVKVPLKKFKSIRETMKEKGLLGEFLRTHKYDPAWKYRFGDLSVTY--EPMA-YMDAAYF | 74 |
| | | \|++\|\|\|\| \|+\|\| + \|\|\|+\| +\|\| \|\|+\| \| \| \| \|\|+ \|+\|\| \|+ | |
| Sbjct: | 1 | FVRIPLKKVPSLREKLSEKGVLLDFLVKRKYEPTKKLTGGASSSRSAVEPLLNYLDAEYY | 60 |
| Query: | 75 | GEISIGTPPQNFLVLFDTGSSNLWVPSVYCQSQ-ACTSHSRFNPSESSTYSTNGQTFSLQ | 133 |
| | | \| \|\|\|\|\|\|\|\| \| \|+\|\|\|\|\|\|+\|\|\|\|\|\|\|\| \| \|\| \| \|+\|\|+\|\|\|\| \| \|\|\|+ | |
| Sbjct: | 61 | GTISIGTPPQKFTVVFDTGSSDLWVPSVYCTSSYACKGHGTFDPSKSSTYKNLGTTFSIS | 120 |
| Query: | 134 | YGSGS-LTGFFGYDTLTVQSIQVPNQEFGLSENEPGTNFVYAQFDGIMGLAYPALSVDE- | 191 |
| | | \|\| \|\| +\|\| \| \|\|+\|\| \| \| \|\|+\|\|\|+ \|\|\|+ \| \| \|\|\|\|+\|\| +\|++ | |
| Sbjct: | 121 | YGDGSSASGFLGQDTVTVGGITVTNQQFGLATKEPGSFFATAVFDGILGLGFPSIEAGGP | 180 |
| Query: | 192 | ATTAMQCMVQEGALTSPVFSVYLSNQQGSSGGAVVFGGVDSSLYTGQIYWAPVTQELYWQ | 251 |
| | | \| + +\| + \|\| \|\|\|\|\| \| +\|\| ++\|\|\|\|\| \| \|\|\| + \| \|\|\| + \|\|\| | |
| Sbjct: | 181 | YTPVFDNLKSQGLIDSPAFSVYL-NSDSGAGGEIIFGGVDPSKYTGSLTWVPVTSQGYWQ | 239 |
| Query: | 252 | IGIEEFLIGGQASGWCSEGCQAIVDTGTSLLTVPQQYMSALLQATGAQEDEY-GQFLVNC | 310 |
| | | \|++ +\|\| + +\|\| \|\|\|\|\|+\|\|\|\|\|\|\| \| +\| + +\| \|\| \|\| \|+++++\| | |
| Sbjct: | 240 | ITLDSITVGGSTT-FCSSGCQAILDTGTSLLYGPTSIVSKIAKAVGASLSEYSGEYVIDC | 298 |
| Query: | 311 | NSIQNLPSLTFIINGVEFPLPPSSYILSN-----------------NGQPLWILGDVFLR | 353 |
| | | +\|\| +\|\| +\|\| \| \| + +\|\|\|+\|+\| \| \|\|\|\|\|\|\|\|\|\|\| | |
| Sbjct: | 299 | DSISSLPDITFFIGGAKITVPPSAYVLQPSSGGSDICLSGFQSDDIPGGPLWILGDVFLR | 358 |
| Query: | 354 | SYYSVYDLGNNRVGFATA 371 | |
| | | \| \| \|+\| \|\|\|+\| \| \| | |
| Sbjct: | 359 | SAYVVFDRDNNRIGLAPA 376 | |

The gastric aspartic proteinases (pepsin A, pepsin B, gastricsin/pepsinogen C and chymosin) are synthesized in the gastric mucosa as inactive precursors, known as zymogens. The gastric zymogens each contain a prosegment (i.e. additional residues at the N-terminus of the active enzyme) that serves to stabilize the inactive form and prevent entry of the substrate to the active site. Upon ingestion of food, each of the zymogens is released into the gastric lumen and undergoes conversion into active enzyme in the acidic gastric juice. This activation reaction is initiated by the disruption of electrostatic interactions between the prosegment and the active enzyme moiety at acidic pH values. The conversion of the zymogen into its active form is a complex process, involving a series of conformational changes and bond cleavage steps that lead to the unveiling of the active site and ultimately the removal and dissociation of the prosegment from the active centre of the enzyme. During this activation reaction, both the prosegment and the active enzyme undergo changes in conformation, and the proteolytic cleavage of the prosegment can occur in one or more steps by either an intra- or inter-molecular reaction. This variability in the mechanism of proteolysis appears to be attributable in part to the structure of the prosegment. Because of the differences in the activation mechanisms among the four types of gastric zymogens and between species of the same zymogen type, no single model of activation can be proposed.

Pepsinogen is an inactive precursor of pepsin, a typical aspartic proteinase, synthesized in the chief cells of gastric glands. There are two major groups of pepsinogen, namely pepsinogen A (PGA) and pepsinogen C (PGC) (or progastricsin), and each frequently has isozymogens. The relative extents of expression of the two pepsinogens vary among animal species and, moreover, their biosynthesis is known to be affected by such bioactive peptides as gastrin and secretin; however, the regulation mechanism of pepsinogen biosynthesis, hence pepsinogen gene expression is not yet clear. Therefore, it is thought to be of fundamental importance to elucidate the primary structures of the pepsinogen gene for such studies. The organization of the human PGA and PGC genes and rat PGC gene is essentially the same; each gene was found to be separated into nine exons by eight introns of various lengths, encoding the amino acid sequence of the corresponding prepepsinogen. These results show that these genes are all derived from a common ancestral gene. The 5′-flanking region of human PGA gene, however, was different from those of human and rat PGC genes, whereas those of human and rat PGC genes were similar to each other. Thus, it is suggested that the expression of the PGA and PGC genes are somewhat differently regulated. Comparative analysis of the genes for the human aspartic proteinases pepsinogen A, pepsinogen C, cathepsin D, cathepsin E and renin reveals a high degree of similarity with regard to their respective coding sequences and the location of exon-intron junctions.

Despite strong conservation of the regions containing the active site aspartyl groups, genetic polymorphisms have been identified for each of the proteinase genes with the exception of cathepsin D. These genetic polymorphisms are useful for localization of genes on linkage maps as well as determination of gene copy number. The chromosomal location of each aspartyl proteinase has been determined by a variety of gene mapping methods employing recombinant DNA probes including analysis of somatic cell hybrid mapping panels, in situ hybridization to metaphase chromosome preparations and family linkage analysis with polymorphic markers. Pepsinogen A exhibits the most extensive polymorphism among aspartic proteinases which can be detected by either by protein electrophoresis or by DNA analysis. Southern blot hybridization with respective DNA probes and polymerase chain reaction (PCR) amplification have revealed nucleotide differences located within the coding and noncoding portions of the aspartic proteinase genes. These polymorphisms can be used to investigate potential roles of each proteinase in genetically influenced clinical conditions. The development of additional highly polymorphic markers detected by PCR amplification of divergent nucleotide sequence repeats will greatly assist with documentation of the effect of genetic variation of the aspartic proteinases may have in specific clinical diseases such as ulcer and hypertension. PGC gene polymorphism has been associated with gastric ulcer and can be a subclinical marker of the genetic predisposition to gastric ulcer. The serum determination of pepsinogen A (PGA) and pepsinogen C (PGC) might indicate gastric mucosal inflammation and atrophy. Body gastric mucosa produces both PGA and PGC, while antral mucosa produces only PGC. Therefore, diseases involving mainly the antrum, such as H. pylori infection, are mainly indicated by the variations in serum PGC than in serum PGA. In agreement, when the antral mucosa is infected by the more virulent cagA positive H. pylori strains, which cause severe inflammation, serum PGC significantly increases.

The protein similarity information, expression pattern, and map location for the NOV17 protein and nucleic acid disclosed herein suggest that it may have important structural and/or physiological functions characteristic of the eukaryotic aspartyl protease family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention may have efficacy for treatment of patients suffering from: ulcer, hypertension (Scand J Clin Lab Invest Suppl 1992;210:111-9), gastric mucosal inflammation and atrophy, and other diseases, disorders and conditions of the like. PGC gene polymorphism has been associated with gastric ulcer and can be a subclinical marker of the genetic predisposition to gastric ulcer (Nippon Rinsho 1996 April;54(4): 1149-54). The serum determination of pepsinogen A (PGA) and pepsinogen C (PGC) might indicate gastric mucosal inflammation and atrophy. Body gastric mucosa produces both PGA and PGC, while antral mucosa produces only PGC. Therefore, diseases involving mainly the antrum, such as H. pylori infection, are mainly indicated by the variations in serum PGC than in serum PGA. In agreement, when the antral mucosa is infected by the more virulent caga positive H. pylori strains, which cause severe inflammation, serum PGC significantly increases (Recenti Prog Med 1999 June;90(6):342-6).

The novel nucleic acid encoding the pepsinogen C-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV10 protein has multiple hydrophilic regions, each of which can be used as an immunogen. In one embodiment, a contemplated NOV17 epitope is from about amino acids 30 to 60. In another embodiment, a contemplated NOV17 epitope is from about amino acids 110 to 130. In other specific embodiments, contemplated NOV17 epitopes are from about amino acids 160 to 170, 180 to 181, 201 to 202, 204 to 205, 207 to 208, 240 to 252, 290 to 310, 340 to 345 or 360 to 365.

NOV18

A disclosed NOV18 nucleic acid (designated CuraGen Acc. No. CG56294-01) encoding a novel ARL-like protein includes 14859 nucleotides (SEQ ID NO: 55) and is shown in Table 18A. An open reading frame was identified beginning with an ATG initiation codon at nucleotides 1-3 and ending with a TAA codon at nucleotides 14857-14859. The start and stop codons are in bold letters in Table 18A.

TABLE 18A

NOV18 Nucleotide Sequence

(SEQ ID NO:55)

ATGTCCCCTCCACCTGAAGAGTCACCCATGTCTCCACCACCGGAGGCATCTCGTCTGTTCCCACCATTTGAAG
AGTCTCCTCTGTCCCCTCCACCTGAGGAGTCTCCCCTTTCCCCACCACCTGAGGCATCACGCCTGTCCCCACC
ACCTGAGGACTCGCCTATGTCCCCACCACCTGAAGAATCACCTATGTCCCCCCCACCTGAGGTATCGCGCCTA
TCCCCCCTGCCTGTGGTGTCACGCCTGTCTCCACCGCCTGAGGAATCTCCCTTGTCCCCACCGCCTGAGGAGT
CTCCCACGTCCCCTCCACCTGAGGCTTCACGCCTCTCCCCACCACCTGAGGACTCCCCCACATCCCCACCACC
TGAGGACTCACCTGCTTCCCCACCACCGGAGGACTCGCTCATGTCCCTGCCGCTGGAGGAGTCACCCCTGTTG
CCACTACCTGAGGAGCCGCAACTCTGCCCCCGGTCCGAGGGGCCGCACCTGTCACCCCGGCCTGAGGAGCCGC
ACCTGTCCCCCCGGCCTGAGGAGCCACACCTATCTCCGCAGGCTGAGGAGCCACACCTGTCCCCCCAGCCTGA
GGAGCCATGCCTATGCGCTGTGCCTGAGGAGCCACACTTGTCCCCCCAGGCTGAGGGACCACATCTGTCCCCT
CAGCCTGAGGAATTGCACCTGTCCCCCCAGACTGAGGAGCCGCACCTGTCTCCTGTGCCTGAGGAGCCATGCT
TGTCCCCCCAACCTGAGGAATCACACCTGTCCCCCCAGTCTGAGGAGCCATGCCTGTCCCCCCGGCCTGAGGA
ATCGCATCTGTCCCCTGAGCTTGAGAAGCCACCCCTGTCCCCTCGGCCTGAAAAGCCCCCTGAGGAGCCAGGC
CAATGCCCTGCACCTGAGGAGCTGCCCTTGTTCCCTCCCCCTGGGGAACCATCCTTATCTCCCTTGCTTGGAG
AGCCAGCCCTGTCTGAGCCTGGGGAACCACCTCTGTCCCCTCTGCCCGAGGAGCTGCCGTTGTCCCCATCTGG
GGAGCCATCCTTGTCGCCTCAGCTGATGCCACCAGATCCCCTTCCTCCTCCACTCTCACCCATTATCACAGCT
GCGGCCCCACCGGCCCTGTCTCCTTTGGGGGAGTTAGAGTACCCCTTTGGTGCCAAAGGGGACAGTGACCCTG
AGTCACCGTTGGCTGCCCCCATCCTGGAGACACCCATCAGCCCTCCACCAGAAGCTAACTGCACTGACCCTGA
GCCTGTCCCCCCTATGATCCTTCCCCCATCTCCAGGCTCCCCAGTGGGGCCGGCTTCTCCCATCCTGATGGAG
CCCCTTCCTCCTCAGTGTTCGCCACTCCTTCAGCATTCCCTGGTTCCCCAAAACTCCCCTCCTTCCCAGTGCT
CTCCTCCTGCCCTACCACTGTCCGTTCCCTCCCCGTTGAGTCCCATAGGGAAGGTAGTGGGGGTCTCAGATGA
GGCTGAGCTGCACGAGATGGAGACTGAGAAAGTTTCAGAACCTGAATGCCCAGCCTTGGAACCCAGTGCCACC
AGTCCTCTCCCTTCCCCAATGGGGGACCTTTCCTGCCCCGCCCCCAGCCCTGCCCCAGCCCTGGATGACTTCT
CTGGCCTAGGGGAAGACACAGCCCCTCTGGATGGGATTGATGCTCCGGGTTCACAGCCAGAGCCTGGACAGAC
CCCTGGCAGTTTGGCTAGTGAACTTAAAGGCTCCCCTGTGCTCCTGGACCCCGAGGAGCTGGCCCCTGTGACC
CCTATGGAGGTCTACCCCGAATGCAAGCAGACAGCAGGGCGGGGCTCACCATGTGAAGAACAGGAAGAGCCAC
GTGCACCGGTGGCCCCCACACCACCCACTCTCATCAAATCCGACATCGTTAACGAGATCTCTAATCTGAGCCA
GGGTGATGCCAGTGCCAGTTTTCCTGGCTCAGAGCCCCTCCTGGGCTCTCCAGACCCGGAGGGGGGTGGCTCC
CTGTCCATGGAGTTGGGGGTCTCTACGGATGTTAGTCCAGCCCGAGATGAGGGCTCCCTACGGCTCTGTACTG
ACTCACTGCCAGAGACTGATGACTCACTATTGTGCGATGCTGGGACAGCTATCAGCGGAGGCAAAGCTGAGGG
GGAGAAGGGGCGGCGGCGCAGCTCCCCAGCCCGTTCCCGCATCAAACAGGGTCGCAGCAGCAGTTTCCCAGGA
AGACGCCGGCCTCGTGGAGGAGCCCATGGAGGGCGTGGTAGAGGACGGGCCCGGCTAAAGTCAACTGCTTCTT
CCATTGAGACTCTGGTAGTTGCTGACATTGATAGCTCTCCCAGTAAGGAGGAGGAGGAAGAAGATGATGACAC
CATGCAGAATACCGTGGTTCTCTTCTCCAACACAGACAAATTTGTCCTAATGCAGGACATGTGTGTGGTATGT
GGCAGCTTTGGCCGGGGGGCAGAGGGCCACCTCCTTGCCTGTTCGCAGTGCTCTCAGTGCTATCACCCTTACT
GTGTCAACAGCAAGATCACCAAGGTGATGCTGCTCAAGGGCTGGCGTTGTGTGGAGTGTATTGTGTGTGAGGT
GTGTGGCCAGGCCTCCGACCCCTCACGCCTGCTGCTCTGTGATGACTGTGATATTAGCTACCACACATACTGC
CTGGACCCCCCACTGCTCACCGTCCCCAAGGGCGGCTGGAAGTGCAAGTGGTGTGTGTCCTGTATGCAGTGTG
GGGCTGCTTCCCCTGGCTTCCACTGTGAATGGCAGAATAGTTACACACACTGTGGGCCCTGTGCCAGCCTGGT
GACCTGCCCTATCTGTCATGCTCCTTACGTAGAAGAGGACCTACTAATCCAGTGCCGCCACTGTGAACGGTGG
ATGCATGCAGGCTGTGAGAGCCTCTTCACAGAGGACGATGTGGACCACGCACCCGATGAAGGCTTTGACTGTG
TCTCCTGCCAGCCCTACGTGGTAAAGCCTGTGGCGCCTGTTGCACCTCCAGAGCTGGTGCCCATGAAGGTGAA
AGAGCCAGAGCCCCAGTACTTTCGCTTCGAAGGCGTGTGGCTGACAGAAACTGGCATGGCCTTGCTGCGTAAC
CTGACCATGTCACCACTGCACAAGCGGCGCCAACGGCGAGGACGGCTTGGCCTCCCAGGCGAGGCAGGATTGG
AGGGTTCTGAGCCCTCAGATGCCCTTGGCCCTGATGACAAGAAGGATGGGGACCTGGACACCGATGAGCTGCT
CAAGGGTGAAGGTGGTGTGGAGCACATGGAGTGCGAAATTAAACTGGAGGGCCCCGTCAGCCCTGATGTGGAG
CCTGGCAAAGAGGAGACCGAGGAAAGCAAAAAACGCAAGCGTAAACCATATCGGCCTGGCATTGGTGGTTTCA
TGGTGCGACAGCGGAAATCCCACACACGCACGAAAAAGGGGCCTGCTGCACAGGCGGAGGTGTTGAGTGGGGA
TGGGCAGCCCGACGAGGTGATACCTGCTGACCTGCCTGCAGAGGGCGCCGTGGAGCAGAGCTTAGCTGAAGGG
GATGAGAAGAAGAAGCAACAGCGGCGAGGGCGCAAGAGGAGCAAACTGGAGGGCATGTTCCCTGCTTACTTGC
AGGAAGCCTTCTTTGGGAAGGAGCTGCTGGACCTGAGCCGTAAGGCCCTTTTTGCAGTTGGGGTGGGCCGGCC
AAGCTTTGGACTAGGGACCCCAAAAGCCAAGGGAGATGGAGGCTCAGAAAGGAAGGAACTCCCCACATCGCAG
AAAGGAGATGATGGTCCAGATATTGCAGATGAAGAATCCCGTGGCCTCGAGGGCAAAGCCGATACACCAGGAC
CTGAGGATGGGGGCGTGAAGGCATCCCCAGTGCCCAGTGACCCTGAGAAGCCAGGCACCCCAGGTGAAGGGAT
GCTTAGCTCTGACTTAGACAGGATTTCCACAGAAGAACTGCCCAAGATGGAATCCAAGGACCTGCAGCAGCTC
TTCAAGGATGTTCTGGGCTCTGAACGAGAACAGCATCTGGGTTGTGGAACCCCTGGCCTAGAAGGCAGCCGTA
CGCCACTGCAGAGGCCCTTTCTTCAAGGTGGACTCCCTTTGGGCAATCTGCCCTCCAGCAGCCCAATGGACTC
CTACCCAGGCCTCTGCCAGTCCCCGTTCCTGGATTCTAGGGAGCGCGGGGGCTTCTTTAGCCCGGAACCCGGT
GAGCCCGACAGCCCCTGGACGGGCTCAGGTGGCACCACGCCCTCCACCCCCACAACCCCCACCACGGAGGGTG
AGGGCGACGGACTCTCCTATAACCAGCGGAGTCTTCAGCGCTGGGAGAAGGATGAGGAGTTGGGCCAGCTGTC
CACCATCTCGCCTGTGCTCTATGCCAACATTAATTTTCCTAATCTCAAGCAAGACTACCCAGACTGGTCAAGC
CGTTGCAAACAAATCATGAAGCTCTGGAGAAAGGTTCCAGCAGCTGACAAAGCCCCCTACCTGCAAAAGGCCA
AAGATAACCGGGCAGCTCACCGCATCAACAAGGTGCAGAAGCAGGCTGAGAGCCAGATCAACAAGCAGACCAA
GGTGGGCGACATAGCCCGTAAGACTGACCGACCGGCCCTACATCTCCGCATTCCCCCGCAGCCAGGGGCACTG
GGCAGCCCGCCCCCCGCTGCTGCCCCCACCATTTTCATTGGCAGCCCCACTACCCCCGCCGGCTTGTCTACCT
CTGCGGACGGGTTCCTGAAGCCGCCGGCGGGCTCGGTGCCTGGCCCTGACTCGCCTGGTGAGCTCTTCCTCAA
GCTCCCACCCCAGGTGCCCGCCCAAGCGCCTTCGCAGGACCCCTTTGGACTGGCCCCTGCCTATCCCCTGGAG
CCCCGCTTCCCCACGGCACCGCCCACCTATCCCCCCTATCCTAGTCCTACGGGGGCCCCTGCGCAGCCCCCGA
TGCTGGGCGCCTCATCTCGTCCTGGGGCTGGCCAGCCAGGGGAATTCCACACTACCCCACCTGGCACCCCCAG
ACACCAGCCCTCCACACCTGACCCGTTCCTCAAACCCCGCTGCCCCTCGCTGGATAACTTGGCTGTGCCTGAG
AGCCCTGGGGTAGGGGGAGGCAAAGCTTCCGAGCCCCTGCTCTCGCCCCCACCTTTTGGGGAGTCCCGGAAGG
CCCTAGAGGTGAAGAAGGAAGAGCTTGGGGCATCCTCTCCTAGCTATGGGCCCCCAAACCTGGGCTTTGTTGA
CTCACCCTCCTCAGGCACCCACCTGGGTGGCCTGGAGTTAAAGACACCTGATGTCTTCAAAGCCCCCCTGACC
CCTCGGGCATCTCAGGTAGAGCCCCAGAGCCCGGGCTTGGGCCTAAGGCCCCAGGAGCCACCCCCTGCCCAGG
CTTTGGCACCTTCTCCTCCAAGTCACCCAGACATCTTTCGCCCTGGCTCCTACACTGACCCATATGCTCAGCC
CCCATTGACTCCTCGGCCCCAACCTCCGCCCCCTGAGAGCTGCTGTGCTCTGCCCCCTCGCTCACTGCCCTCC
GACCCTTTCTCCCGAGTGCCTGTCAGTCCTCAGTCCCAGTCCAGCTCCCAGTCTCCACTGACACCCCGGCCTC
TGTCTGCTGAAGCTTTTTGCCCATCACCCGTTACCCCTCGCTTCCAGTCCCCTGACCCTTATTCTCGCCCACC
CTCACGCCCTCAGTCCCGTGACCCATTTGCCCCATTGCATAAGCCACCCCGACCCCAGCCCCCTGAAGTTGCC
TTTAAGGCTGGGTCTCTAGCCCACACTTCGCTGGGGGCTGGGGGGTTCCCAGCAGCCCTGCCCGCGGGGCCAG
CAGGTGAGCTCCATGCCAAGGTCCCAAGTGGGCAGCCCCCCAATTTTGTCCGGTCCCCTGGGACGGGTGCATT
TGTGGGCACCCCCTCTCCCATGCGTTTCACTTTCCCTCAGGCAGTAGGGGAGCCTTCCCTAAAGCCCCCTGTC
CCTCAGCCTGGTCTCCCGCCACCCCATGGGATCAACAGCCATTTTGGGCCCGGCCCCACCTTGGGCAAGCCTC
AAAGCACAAACTACACAGTAGCCACAGGGAACTTCCACCCATCGGGCAGCCCCCTGGGGCCCAGCAGCGGGTC
CACAGGGGAGAGCTATGGGCTGTCCCCACTACGCCCTCCGTCGGTTCTGCCACCACCTGCACCCGACGGATCC
CTCCCCTACCTGTCCCATGGAGCCTCACAGCGATCAGGCATCACCTCTCCTGTCGAAAAGCGAGAAGACCCAG
GGACTGGAATGGGTAGCTCTTTGGCGACAGCTGAACTCCCAGGTACCCAGGACCCAGGCATGTCCGGCCTTAG
CCAAACAGAGCTGGAGAAGCAACGGCAGCGCCAGCGGCTACGAGAGCTGCTGATTCGGCAGCAGATCCAGCGC
AACACCCTGCGGCAGGAGAAGGAAACAGCTGCAGCAGCTGCGGGAGCAGTGGGGCCTCCAGGCAGCTGGGGTG
CTGAGCCCAGCAGCCCTGCCTTTGAGCAGCTGAGTCGAGGCCAGACCCCCTTTGCTGGGACACAGGACAAGAG
CAGCCTTGTGGGGTTGCCCCCAAGCAAGCTGAGTGGCCCCATCCTGGGGCCAGGGTCCTTCCCTAGCGATGAC
CGACTCTCCCGGCCACCTCCACCAGCCACGCCTTCCTCTATGGATGTGAACAGCCGGCAACTGGTAGGAGGCT
CCCAAGCTTTCTATCAGCGAGCACCCTATCCTGGGTCCCTGCCCTTACAGCAGCAACAGCAACAACTGTGGCA
GCAACAACAGGCAACAGCAGCAACCTCCATGCGATTTGCCATGTCAGCTCGCTTTCCATCAACTCCTGGACCT
GAACTTGGCCGCCAAGCCCTAGGTTCCCCGTTGGCGGGAATTTCCACCCGTCTGCCAGGCCCTGGTGAGCCAG
TGCCTGGTCCAGCTGGTCCTGCCCAGTTCATTGAGCTGCGGCACAATGTACAGAAAGGACTGGGACCTGGGGG
CACTCCGTTTCCTGGTCAGGGCCCACCTCAGAGACCCCGTTTTTACCCTGTAAGTGAGGACCCCCACCGACTG
GCTCCTGAAGGGCTTCGGGGCCTGGCGGTATCAGGTCTTCCCCCACAGAAACCCTCAGCCCCACCGGCCCCTG
AATTGAACAACAGTCTTCATCCAACACCCCACACCAAGGGTCCTACCCTGCCAACTGGTTTGGAGCTGGTCAA
CCGGCCCCCGTCGAGCACTGAGCTTGGCCGCCCCAATCCTCTGGCCCTGGAAGCTGGGAAGTTGCCCTGTGAG
GATCCCGAGCTGGATGACGATTTTGATGCCCACAAGGCCCTAGAGGATGATGAAGAGCTTGCTCACCTGGGTC
TGGGTGTGGATGTGGCCAAGGGTGATGATGAACTTGGCACCTTAGAAAACCTGGAGACCAATGACCCCCACTT
GGATGACCTGCTCAATGGAGACGAGTTTGACCTGCTGGCATATACTGATCCTGAGCTGGACACTGGGGACAAG
AAGGATATCTTCAATGAGCACCTGAGGCTGGTAGAATCGGCTAATGAGGAGGCTGAACGGGAGGCCCTGCTGC
GGGGGGTGGAGCCAGGACCCTTGGGCCCTGAGGAGCGCCCTCCCCCTGCTGCTGATGCCTCTGAACCCCGCCT
GGCATCTGTGCTCCCTGAGGTGAAGCCCAAGGTGGAGGAGGGTGGACGCCACCCTTCTCCTTGCCAATTCACC
ATTGCTACCCCCAAGGTAGAGCCCGCACCTGCTGCCAATTCCCTTGGCCTGGGGCTAAAGCCAGGACAGAGCA
TGATGGGCAGCCGGGATACCCGGATGGGCACAGGGCCATTTTCTAGCAGTGGGCACACAGCTGAGAAGGCCTC
CTTTGGGGCCACGGGAGGGCCACCAGCTCACCTGCTGACCCCCAGCCCACTGAGTGGCCCAGGAGGATCCTCC
CTGCTGGAAAAGTTTGAGCTCGAGAGTGGGGCTTTGACCTTGCCTGGTGGACCTGCAGCATCTGGGGATGAGC
TAGACAAGATGGAGAGCTCACTGGTAGCCAGCGAGTTACCCCTGCTCATTGAGGACCTGTTGGAGCATGAGAA
GAAGGAGCTGCAGAAGAAGCAGCAGCTTTCAGCACAGTTGCAGCCTGCCCAGCAGCAGCAGCAACAGCAGCAG
CAGCATTCCCTACTGCCTGCACCAGGCCCTGCCCAGGCCATGTCTTTGCCACATGAGGGCTCTTCTCCCAGTT
TGGCTGGGTCCCAACAGCAGCTTTCCCTGGGTCTTGCAGTTGCCCGACAGCCAGGTTTGCCCCAGCCACTGAT
GCCCACCCAGCCACCAGCTCATGCCCTCCAGCAACGCCTGGCTCCATCCATGGCTATGGTGTCCAATCAAGGG
CATATGCTAAGTGGGCAGCATGGAGGGCAGGCAGGCTTGGTACCCCAGCAGAGCTCACAGCCAGTGCTATCAC
AGAAGCCCATGGGCACCATGCCACCTTCCATGTGCATGAAGCCGCAGCAATTGGCAATGCAGCAGCAGCTGGC
AAACAGCTTCTTCCCAGATACAGACCTGGACAAATTTGCTGCAGAAGATATCATTGGTCCCATTGCAAAGGCC
AAGATGGTGGCTTTGAAAGGCATCAAGAAAGTGATGGCTCAGGGCAGCATTGGGGTGGCACCTGGTATGAACA
GACAGCAAGTGTCTCTGCTAGCCCAGAGGCTCTCGGGGGGACCTAGCAGTGATCTGCAGAACCATGTGGCAGC
TGGGAGTGGCCAGGAGCGGAGTGCTGGTGATCCCTCCCAGCCTCGTCCCAACCCGCCCACTTTTGCTCAGGGA
GTGATCAATGAAGCTGACCAGCGGCAGTATGAGGAGTGGCTGTTCCATACCCAGCAGCTCCTACAGATGCAGC
TGAAGGTGCTAGAGGAGCAGATTGGTGTACACCGCAAGTCCCGGAAGGCTCTGTGTGCCAAGCAGCGCACTGC
CAAAAAAGCTGGCCGTGAGTTCCCAGAAGCTGATGCTGAGAAGCTCAAGCTGGTTACAGAGCAGCAGAGCAAG
ATCCAGAAACAACTGGATCAGGTCCGGAAACAGCAGAAGGAGCACACTAATCTCATGGCAGAATATCGGAACA
AGCAGCAGCAACAACAGCAGCAGCAGCAGCAACAACAGCAACAGCACTCAGCTGTGCTGGCTCTCAGCCCTTC
CCAGAGTCCCCGGCTGCTCACCAAGCTCCCTGGTCAGCTGCTCCCTGGCCATGGGCTGCAGCCACCACAGGGG
CCTCCGGGTGGGCAAGCCGGAGGTCTTCGCCTGACCCCTGGGGGTATGGCACTACCTGGACAGCCTGGTGGCC
CCTTCCTTAATACAGCTCTGGCCCAACAGCAGCAACAGCAACATTCTGGTGGGGCTGGATCCCTGGCTGGCCC
TTCAGGGGGCTTCTTCCCTGGCAACCTTGCTCTTCGAAGCCTCGGACCTGATTCAAGGCTTTTACAGGAAAGG
CAGCTGCAGCTGCAGCAGCAACGTATGCAGCTGGCCCAGAAACTGCAGCAGCAGCAGCAGCAGCAACAGCAGC
AGCAGCACCTTCTAGGACAGGTGGCAATCCAGCAGCAACAGCAGCAGGGTCCTGGAGTACAGACAAACCAAGC
TCTGGGTCCCAAGCCCCAGGGCCTTATGCCTCCCAGCAGCCACCAAGGCCTCCTGGTCCAGCAGCTGTCCCCT
CAACCACCCCAGGGGCCCCAGGGCATGCTGGGCCCTGCCCAGGTGGCTGTGTTGCAGCAGCAGCACCCTGGAG
CTTTGGGCCCCCAGGGCCCTCACAGACAGGTGCTTATGACCCAGTCCCGGGTGCTCAGTTCCCCCCAGCTGGC
ACAGCAGGGTCAGGGCCTTATGGGACACAGGCTGGTCACAGCCCAGCAGCAGCAGCAGCAACAACAGCACCAA
CAGCAAGGGTCCATGGCAGGGCTGTCCCATCTTCAGCAAAGTCTGATGTCACACAGTGGGCAGCCCAAACTGA
GCGCTCAGCCCATGGGCTCTTTACAGCAGCTTCAGCAGCAGCAGCAGCTGCAACAGCAACAGCAACTTCAGCA
GCAGCAGCAGCAGCAGCTACAACAGCAACAGCAACTTCAGCAGCAACAGCTTCAACAGCAGCAACAGCAGCAG
CAGCTTCAACAACAGCAGCAGCAACAGCTTCAACAGCAGCAACAGCAGCTACAACAGCAACAGCAACAACAAC
AGCAGCAGTTTCAACAGCAGCAGCAACAGCAGCAGATGGGCCTTTTAAACCAGAGTCGAACTTTACTGTCCCC
TCAGCAACAACAGCAGCAGCAAGTGGCACTTGGCCCTGGCATGCCAGCAAAGCCTCTTCAACACTTTTCTAGC
CCTGGAGCCCTGGGTCCAACCCTCCTCCTGACGGGCAAGGAACAAAACACCGTAGACCCAGCCGTTTCTTCAG
AGGCCACTGAGGGGCCCTCTACACATCAGGGAGGGCCGTTAGCAATAGGAACTACCCCTGAGTCAATGGCCAC
TGAACCAGGAGAGGTAAAGCCCTCACTCTCTGGGGACTCACAACTCCTGCTTGTCCAACCCCAGCCCCAGCCT
CAGCCCAGCTCTCTGCAGCTGCAGCCACCTCTGAGGCTTCCAGGACAACAGCAGCAGCAAGTTAGCCTGCTCC
ACACAGCAGGTGGAGGAAGCCATGGGCAGCTAGGCAGTGGATCATCTTCTGAGGCCTCATCTGTGCCCCACCT
GCTGGCTCAGCCCTCTGTTTCCTTAGGGGATCAGCCTGGGTCCATGACCCAGAACCTTCTGGGCCCCCAACAG
CCCATGCTAGAGCGGCCCATGCAAAATAATACAGGGCCACAACCTCCCAAACCAGGACCTGTCCTCCAGTCTG
GGCAGGGTCTGCCTGGGGTTGGAATCATGCCTACGGTGGGTCAGCTTCGAGCACAGCTCCAAGGAGTCCTGGC
CAAAAACCCACAGCTGCGGCACTTAAGTCCTCAGCAGCAGCAGCAGCTACAGGCACTCCTCATGCAGCGGCAG
CTGCAGCAGAGTCAGGCAGTACGCCAGACCCCACCCTACCAGGAGCCTGGGACCCAGACCTCTCCCCTCCAGG
GCCTCCTGGGCTGCCAACCTCAACTTGGGGGCTTCCCTGGACCACAGACAGGCCCCCTCCAGGAGCTAGGGGC
AGGGCCTCGACCTCAGGGCCCACCCCGGCTCCCTGCCCCACCAGGAGCCTTATCTACAGGACCAGTCCTTGGC
CCTGTCCATCCCACACCTCCACCATCCAGCCCTCAAGAGCCAAAGAGACCTTCACAATTACCTTCCCCCAGCT
CCCAGCTTCCCACTGAGGCCCAGCTCCCTCCCACCCATCCAGGGACCCCCAAACCTCAGGGGCCAACCTTGGA
GCCGCCTCCTGGGAGGGTCTCACCTGCTGCTGCCCAGCTTGCAGATACCTTGTTTAGCAAGGGTCTGGGACCT
TGGGATCCCCCAGACAACCTAGCAGAAACCCAGAAGCCAGAGCAGAGCAGCCTGGTACCTGGGCATCTGGACC
AGGTGAATGGACAGGTGGTGCCTGAGGCATCCCAACTCAGCATCAAGCAGGAACCTCGGGAAGAGCCATGTGC
CCTGGGAGCCCAGTCAGTGAAGAGGGAGGCCAATGGGGAGCCAATAGGGGCACCAGGAACCAGCAACCACCTC
CTGCTGGCAGGCCCTCGCTCAGAAGCTGGGCATCTGCTCTTGCAGAAGCTACTCCGGGCAAAGAATGTGCAAC
TCAGCACTGGGCAGGGGTCCGAGGGGCTGCGAGCTGAGATCAACGCGGGCATTGACAGCAAGCTGGCTGGGCT
GGAGCAGAAACTACAGGGTACCCCCAGCAACAAGGAGGATGCAGCAGCAAGGAAGCCTTTGACACCGAAGCCC
AAGCGGGTACAGAAGGCAAGCGACAGGTTGGTGAGCTCCCGAAAGAAGCTGCGGAAGGAGGACGGCGTCAGGG
CCAGCGAGGCCTTGCTGAAACAGCTGAAACAGGAGCTGTCCCTGCTGCCCCTAACGGAGCCTGCTATCACCGC
CAATTTTAGCCTCTTTGCCCCCTTTGGCAGTGGCTGCCCAGTCAATGGGCAGAGCCAGCTGAGGGGGGCCTTT
GGAAGTGGGGCGCTGCCCACTGGCCCTGACTACTATTCCCAGCTGCTTACCAAGAATAACCTGAGTAACCCGC
CGACACCACCCTCGTCGCTGCCCCCCACCCCACCCCCATCGGTGCAGCAGAAGATGGTGAATGGCGTCACCCC
ATCTGAAGAGCTGGGGGAGCACCCCAAGGATGCTGCCTCTGCCCGGGATAGTGAAAGGGCACTGAGGGATACT
TCAGAGGTGAAGAGTCTAGACCTGCTGGCTGCCTTGCCTACACCCCCTCACAATCAGACTGAGGATGTCAGGA
TGGAGAGTGATGAGGATAGCGATTCTCCTGACAGCATTGTGCCAGCTTCATCCCCTGAGAGCATCTTGGGGGA
GGAGGCCCCTCGTTTCCCTCATCTGGGCTCAGGCCGGTGGGAGCAAGAGGACCGGGCCCTCTCCCCTGTCATC
CCCCTCATTCCTCGGGCCAGCATCCCAGTCTTCCCAGATACCAAACCTTATGGGGCCCTTGGCCTGGACGTCC
CTGGAAAGCTGCCTGTCACAACTTGGGAAAAGGGCAAAGGAAGTGAGGTGTCAGTCATGCTCACAGTCTCTGC
TGCTGCAGCCAAGAACCTGAATGGCGTGATGGTGGCAGTGGCGGAGCTGCTGAGCATGAAGATCCCCAACTCC
TATGAGGTGCTGTTCCCAGAGAGCCCCGCCCGGGCAGGCACTGAGCCAAAGAAGGGGGAAGCTGAGGGTCCTG
GTGGGAAGGAAAAGGGTCTGGAAGGCAAGAGCCCAGACACTGGCCCTGATTGGCTGAAGCAGTTTGATGCAGT
GTTGCCTGGCTATACCCTGAAGAGCCAACTAGACATCTTGAGCCTCCTGAAACAGGAGAGCCCCGCCCCAGAG
CCACCCACTCAGCACAGCTATACCTACAATGTCTCCAATCTGGATGTGCGACAGCTCTCGGCCCCACCTCCTG
AAGAACCCTCCTTGGCACCTTCTCCTGCCAGTCCCCCTACTGAGCCCTTGGTTGAACTTCCCACCGAACCCTT
GGCTGAGCCACCCGTCCCCTCACCTCTGCCACTGGCCTCATCCCCTGAATCAGCCCGACCCAAGCCCCGTGCC
CGGCCCCCTGAAGAAGGTGAAGATACCCGTCCTCCTCGCCTCAAGAAATGGAAAGGAGTGCGCTGGAAGCGGC
TTCGGCTGCTGCTGACCATCCAGAAGGGCAGTGGACGGCAGGAGGATGAGCGGGAAGTGGCAGAGTTTATGGA
GCAGCTTGGCACAGCCTTGCGACCTGACAAGGTACCGCGAGACATGCGTCGCTGCTGTTTCTGTCATGAGGAG
GGTGACGGGGCCACTGATGGGCCTGCCCGTCTGCTGAACCTGGACCTGGACCTGTGGGTGCACCTCAACTGTG
CCCTTTGGTCCACGGAGGTGTATGAGACCCAGGGCGGAGCACTGATGAATGTGGAGGTTGCCCTGCACCGAGG
ACTGCTAACCAAGTGCTCCCTGTGCCAGCGAACTGGTGCCACCAGCAGCTGCAATCGCATGCGTTGCCCCAAT
GTCTACCATTTTGGTTGTGCCATCCGCGCCAAGTGCATGTTCTTCAAGGACAAGACCATGCTGTGTCCAATGC
ATAAGATCAAGGGGCCCTGTGAGCAAGAGCTGAGCTCTTTTGCTGTCTTCCGGCGGGTCTACATTGAGCGGGA
CGAGGTGAAGCAAATCGCTAGCATCATTCAGCGGGGAGAACGGCTGCACATGTTCCGTGTGGGGGGGCTTGTG
TTCCACGCCATCGGACAGCTGCTGCCTCACCAGATGGCTGACTTTCATAGTGCCACTGCCCTCTATCCCGTGG
GCTACGAGGCCACGCGCATCTATTGGAGCCTCCGCACCAACAATCGTCGCTGCTGCTATCGCTGTTCTATTGG
TGAGAACAACGGGCGGCCGGAGTTTGTAATCAAAGTCATCGAGCAGGGCCTGGAGGACCTGGTCTTCACTGAC
GCCTCTCCCCAGGCCGTGTGGAATCGCATCATTGAGCCTGTGGCTGCCATGAGAAAAGAGGCTGACATGCTGC
GACTCTTCCCTGAGTATCTGAAGGGCGAGGAGCTCTTTGGGCTGACGGTGCATGCCGTGCTTCGCATAGCTGA
ATCACTGCCCGGGGTGGAGAGCTGTCAAAACTATTTATTCCGCTATGGGCGCCACCCCCTTATGGAGCTGCCA
CTCATGATCAACCCCACTGGCTGTGCCCGATCAGAGCCTAAAATCCTCACACACTACAAACGGCCCCATACCC
TGAACAGCACCAGCATGTCTAAGGCATATCAGAGCACCTTCACAGGCGAGACCAACACCCCCTACAGCAAGCA
GTTTGTGCACTCCAAGTCATCTCAGTACCGGCGGCTGCGCACCGAATGGAAGAACAACGTGTACCTGGCTCGC
TCCCGTATCCAGGGCCTGGGGCTCTATGCAGCCAAGGACCTAGAAAAGCACACAATGGTTATCGAGTACATTG
GCACCATCATTCGGAACGAGGTGGCCAACCGGCGGGAGAAAATCTACGAAGAGCAGAATCGAGGCATCTACAT
GTTCCGAATAAACAATGAACATGTGATTGATGCTACGTTGACCGGCGGCCCTGCCAGGTACATTAACCATTCC
TGTGCCCCTAACTGTGTGGCCGAAGTCGTGACATTTGACAAAGAGGACAAAATCATCATCATCTCCAGCCGGC
GAATCCCCAAAGGAGAGGAGCTAACCTATGACTATCAGTTTGATTTTGAGGACGATCAGCACGAGATCCCCTG
CCACTGTGGAGCCTGGAATTGTCGGAAATGGATGAACTAA

The nucleic acid sequence of NOVI 8 maps to chromosome 12q12-q14 and has 13081 of 13153 bases (99%) identical to a gb:GENBANK-ID:AF010404|acc:AF010404.1 mRNA from Homo sapiens (Homo sapiens ALR mRNA, complete cds) (E=0.0).

A NOV18 polypeptide (SEQ ID NO: 56) is 4952 amino acid residues and is presented using the one letter code in Table 18B. Signal P, Psort and/or Hydropathy results predict that NOV18 is likely to be localized to the nucleus with a certainty of 0.9800. In other embodiments, NOV18 is localized to the microbody (peroxisome) with a certainty of 0.3000, the mitochondrial matrix space with a certainty of 0.1000 or the lysosomy (lumen) with a certainty of 0.1000.

TABLE 18B

NOV18 protein sequence

(SEQ ID NO:56)

MSPPPEESPMSPPPEASRLFPPFEESPLSPPPEESPLSPPPEASRLSPPPEDSPMSPPPEESPMSPPPEVSRLS
PLPVVSRLSPPPEESPLSPPPEESPTSPPPEASRLSPPPEDSPTSPPPEDSPASPPPEDSLMSLPLEESPLLPL
PEEPQLCPRSEGPHLSPRPEEPHLSPRPEEPHLSPQAEEPHLSPQPEEPCLCAVPEEPHLSPQAEGPHLSPQPE
ELHLSPQTEEPHLSPVPEEPCLSPQPEESHLSPQSEEPCLSPRPEESHLSPELEKPPLSPRPEKPPEEPGQCPA
PEELPLFPPPGEPSLSPLLGEPALSEPGEPPLSPLPEELPLSPSGEPSLSPQLMPPDPLPPPLSPIITAAAPPA
LSPLGELEYPFGAKGDSDPESPLAAPILETPISPPPEANCTDPEPVPPMILPPSPGSPVGPASPILMEPLPPQC
SPLLQHSLVPQNSPPSQCSPPALPLSVPSPLSPIGKVVGVSDEAELHEMETEKVSEPECPALEPSATSPLPSPM
GDLSCPAPSPAPALDDFSGLGEDTAPLDGIDAPGSQPEPGQTPGSLASELKGSPVLLDPEELAPVTPMEVYPEC
KQTAGRGSPCEEQEEPRAPVAPTPPTLIKSDIVNEISNLSQGDASASFPGSEPLLGSPDPEGGGSLSMELGVST
DVSPARDEGSLRLCTDSLPETDDSLLCDAGTAISGGKAEGEKGRRRSSPARSRIKQGRSSSFPGRRRPRGGAHG
GRGRGRARLKSTASSIETLVVADIDSSPSKEEEEEDDDTMQNTVVLFSNTDKFVLMQDMCVVCGSFGRGAEGHL
LACSQCSQCYHPYCVNSKITKVMLLKGWRCVECIVCEVCGQASDPSRLLLCDDCDISYHTYCLDPPLLTVPKGG
WKCKWCVSCMQCGAASPGFHCEWQNSYTHCGPCASLVTCPICHAPYVEEDLLIQCRHCERWMHAGCESLFTEDD
VDHAPDEGFDCVSCQPYVVKPVAPVAPPELVPMKVKEPEPQYFRFEGVWLTETGMALLRNLTMSPLHKRRQRRG
RLGLPGEAGLEGSEPSDALGPDDKKDGDLDTDELLKGEGGVEHMECEIKLEGPVSPDVEPGKEETEESKKRKRK
PYRPGIGGFMVRQRKSHTRTKKGPAAQAEVLSGDGQPDEVIPADLPAEGAVEQSLAEGDEKKKQQRRGRKRSKL
EGMFPAYLQEAFFGKELLDLSRKALFAVGVGRPSFGLGTPKAKGDGGSERKELPTSQKGDDGPDIADEESRGLE
GKADTPGPEDGGVKASPVPSDPEKPGTPGEGMLSSDLDRISTEELPKMESKDLQQLFKDVLGSEREQHLGCGTP
GLEGSRTPLQRPFLQGGLPLGNLPSSSPMDSYPGLCQSPFLDSRERGGFFSPEPGEPDSPWTGSGGTTPSTPTT
PTTEGEGDGLSYNQRSLQRWEKDEELGQLSTISPVLYANINFPNLKQDYPDWSSRCKQIMKLWRKVPAADKAPY
LQKAKDNRAAHRINKVQKQAESQINKQTKVGDIARKTDRPALHLRIPPQPGALGSPPPAAAPTIFIGSPTTPAG
LSTSADGFLKPPAGSVPGPDSPGELFLKLPPQVPAQAPSQDPFGLAPAYPLEPRFPTAPPTYPPYPSPTGAPAQ
PPMLGASSRPGAGQPGEFHTTPPGTPRHQPSTPDPFLKPRCPSLDNLAVPESPGVGGGKASEPLLSPPPFGESR
KALEVKKEELGASSPSYGPPNLGFVDSPSSGTHLGGLELKTPDVFKAPLTPRASQVEPQSPGLGLRPQEPPPAQ
ALAPSPPSHPDIFRPGSYTDPYAQPPLTPRPQPPPPESCCALPPRSLPSDPFSRVPVSPQSQSSSQSPLTPRPL
SAEAFCPSPVTPRFQSPDPYSRPPSRPQSRDPFAPLHKPPRPQPPEVAFKAGSLAHTSLGAGGFPAALPAGPAG
ELHAKVPSGQPPNFVRSPGTGAFVGTPSPMPFTFPQAVGEPSLKPPVPQPGLPPPHGINSHFGPGPTLGKPQST
NYTVATGNFHPSGSPLGPSSGSTGESYGLSPLRPPSVLPPPAPDGSLPYLSHGASQRSGITSPVEKREDPGTGM
GSSLATAELPGTQDPGMSGLSQTELEKQRQRQRLRELLIRQQIQRNTLRQEKETAAAAAGAVGPPGSWGAEPSS
PAFEQLSRGQTPFAGTQDKSSLVGLPPSKLSGPILGPGSFPSDDRLSRPPPPATPSSMDVNSRQLVGGSQAFYQ
RAPYPGSLPLQQQQQQLWQQQQATAATSMRFAMSARFPSTPGPELGRQALGSPLAGISTRLPGPGEPVPGPAGP
AQFIELRHNVQKGLGPGGTPFPGQGPPQRPRFYPVSEDPHRLAPEGLRGLAVSGLPPQKPSAPPAPELNNSLHP
TPHTKGPTLPTGLELVNRPPSSTELGRPNPLALEAGKLPCEDPELDDDFDAHKALEDDEELAHLGLGVDVAKGD
DELGTLENLETNDPHLDDLLNGDEFDLLAYTDPELDTGDKKDIFNEHLRLVESANEEAEREALLRGVEPGPLGP
EERPPPAADASEPRLASVLPEVKPKVEEGGRHPSPCQFTIATPKVEPAPAANSLGLGLKPGQSMMGSRDTRMGT
GPFSSSGHTAEKASFGATGGPPAHLLTPSPLSGPGGSSLLEKFELESGALTLPGGPAASGDELDKMESSLVASE
LPLLIEDLLEHEKKELQKKQQLSAQLQPAQQQQQQQQQHSLLPAPGPAQAMSLPHEGSSPSLAGSQQQLSLGLA
VARQPGLPQPLMPTQPPAHALQQRLAPSMAMVSNQGHMLSGQHGGQAGLVPQQSSQPVLSQKPMGTMPPSMCMK
PQQLAMQQQLANSFFPDTDLDKFAAEDIIGPIAKAKMVALKGIKKVMAQGSIGVAPGMNRQQVSLLAQRLSGGP
SSDLQNHVAAGSGQERSAGDPSQPRPNPPTFAQGVINEADQRQYEEWLFHTQQLLQMQLKVLEEQIGVHRKSRK
ALCAKQRTAKKAGREFPEADAEKLKLVTEQQSKIQKQLDQVRKQQKEHTNLMAEYRNKQQQQQQQQQQQQQQHS
AVLALSPSQSPRLLTKLPGQLLPGHGLQPPQGPPGGQAGGLRLTPGGMALPGQPGGPFLNTALAQQQQQQHSGG
AGSLAGPSGGFFPGNLALRSLGPDSRLLQERQLQLQQQRMQLAQKLQQQQQQQQQQQHLLGQVALQQQQQQGPG
VQTNQALGPKPQGLMPPSSHQGLLVQQLSPQPPQGPQGMLGPAQVAVLQQQHPGALGPQGPHRQVLMTQSRVLS
SPQLAQQGQGLMGHRLVTAQQQQQQQQHQQQGSMAGLSHLQQSLMSHSGQPKLSAQPMGSLQQLQQQQQLQQQQ
QLQQQQQQQLQQQQQLQQQQLQQQQQQQQLQQQQQQQLQQQQQQLQQQQQQQQQQFQQQQQQQQMGLLNQSRTL
LSPQQQQQQQVALGPGMPAKPLQHFSSPGALGPTLLLTGKEQNTVDPAVSSEATEGPSTHQGGPLAIGTTPESM
ATEPGEVKPSLSGDSQLLLVQPQPQPQPSSLQLQPPLRLPGQQQQQVSLLHTAGGGSHGQLGSGSSSEASSVPH
LLAQPSVSLGDQPGSMTQNLLGPQQPMLERPMQNNTGPQPPKPGPVLQSGQGLPGVGIMPTVGQLRAQLQGVLA
KNPQLRHLSPQQQQQLQALLMQRQLQQSQAVRQTPPYQEPGTQTSPLQGLLGCQPQLGGFPGPQTGPLQELGAG
PRPQGPPRLPAPPGALSTGPVLGPVHPTPPPSSPQEPKRPSQLPSPSSQLPTEAQLPPTHPGTPKPQGPTLEPP
PGRVSPAAAQLADTLFSKGLGPWDPPDNLAETQKPEQSSLVPGHLDQVNGQVVPEASQLSIKQEPREEPCALGA
QSVKREANGEPIGAPGTSNHLLLAGPRSEAGHLLLQKLLRAKNVQLSTGQGSEGLRAEINGHIDSKLAGLEQKL
QGTPSNKEDAAARKPLTPKPKRVQKASDRLVSSRKKLRKEDGVRASEALLKQLKQELSLLPLTEPAITANFSLF
APFGSGCPVNGQSQLRGAFGSGALPTGPDYYSQLLTKNNLSNPPTPPSSLPPTPPPSVQQKMVNGVTPSEELGE
HPKDAASARDSERALRDTSEVKSLDLLAALPTPPHNQTEDVRMESDEDSDSPDSIVPASSPESILGEEAPRFPH
LGSGRWEQEDRALSPVIPLIPRASIPVFPDTKPYGALGLEVPGKLPVTTWEKGKGSEVSVMLTVSAAAAKNLNG
VMVAVAELLSMKIPNSYEVLFPESPARAGTEPKKGEAEGPGGKEKGLEGKSPDTGPDWLKQFDAVLPGYTLKSQ
LDILSLLKQESPAPEPPTQHSYTYNVSNLDVRQLSAPPPEEPSLAPSPASPPTEPLVELPTEPLAEPPVPSPLP
LASSPESARPKPRARPPEEGEDTRPPRLKKWKGVRWKRLRLLLTIQKGSGRQEDEREVAEFMEQLGTALRPDKV
PRDMRRCCFCHEEGDGATDGPARLLNLDLDLWVHLNCALWSTEVYETQGGALMNVEVALHRGLLTKCSLCQRTG
ATSSCNRMRCPNVYHFGCAIRAKCMFFKDKTMLCPMHKIKGPCEQELSSFAVFRRVYIERDEVKQIASIIQRGE
RLHMFRVGGLVFHAIGQLLPHQMADFHSATALYPVGYEATRIYWSLRTNNRRCCYRCSIGENNGRPEFVIKVIE
QGLEDLVFTDASPQAVWNRIIEPVAAMRKEADMLRLFPEYLKGEELFGLTVHAVLRIAESLPGVESCQNYLFRY
GRHPLMELPLMINPTGCARSEPKILTHYKRPHTLNSTSMSKAYQSTFTGETNTPYSKQFVHSKSSQYRRLRTEW
KNNVYLARSRIQGLGLYAAKDLEKHTMVIEYIGTIIRNEVANRREKIYEEQNRGIYMFRINNEHVIDATLTGGP
ARYINHSCAPNCVAEVVTFDKEDKIIIISSRRIPKGEELTYDYQFDFEDDQHEIPCHCGAWNCRKWMN

The NOV18 amino acid sequence have 4946 of 4957 amino acid residues (99%) identical to, and 4946 of 4957 amino acid residues (99%) similar to, the 4957 amino acid residue ptnr:SPTREMBL-ACC 014687 protein from Homo sapiens (Human) (ALR) (E=0.0).

NOV 18 is expressed in at least the following tissues: Adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. This information was derived by determining the tissue sources of the sequences that were included in the invention including but not limited to SeqCalling sources, Public EST sources, and/or RACE sources.

NOV18 also has homology to the amino acid sequences shown in the BLASTP data listed in Table 18C.

TABLE 18C
BLAST results for NOV18

| Gene Index/ | | | | | |
|---|---|---|---|---|---|
| Identifier | Protein/Organism | Length (aa) | Identity (%) | Positives (%) | Expect |
| gi\|4505197\|ref\|NP_0 | myeloid/lymphoid | 5262 | 2179/2942 | 2179/2942 | 0.0 |
| 03473.1\| | or mixed-lineage | | (74%) | (74%) | |
| (NM_003482) | leukemia 2; | | | | |
| | ALL1-related gene | | | | |
| | [Homo sapiens] | | | | |
| gi\|7512280\|pir\|\|T03 | ALR protein - | 4957 | 2179/2942 | 2179/2942 | 0.0 |
| 455 | human | | (74%) | (74%) | |
| gi\|14761653\|ref\|XP_{—} | myeloid/lymphoid | 5262 | 2172/2942 | 2174/2942 | 0.0 |
| 028760.1\| | or mixed-lineage | | (73%) | (73%) | |
| (XM_028760) | leukemia 2 [Homo | | | | |
| | sapiens] | | | | |
| gi\|14626492\|gb\|AAK7 | MLL3-like protein | 677 | 398/557 | 467/557 | 0.0 |
| 0214.1\| (AY036887) | [Mus musculus] | | (71%) | (83%) | |
| gi\|3540281\|gb\|AAC34 | All-1 related | 4823 | 434/561 | 495/561 | 0.0 |
| 383.1\| (AF056116) | protein [Takifugu | | (77%) | (87%) | |
| | rubripes] | | | | |

The homology of these sequences is shown graphically in the ClustalW analysis shown in Table 18D.

[image]

[image]

[image]

[image]

[image]

[image]

[image]

[image]

[image]

[image]

[image]

[image]

[image]

[image]

[image]

[image]

Tables 18E, 18F, 18G and 18H list the domain description from DOMAIN analysis results against NOV18. This indicates that the NOV18 sequence has properties similar to those of other proteins known to contain these domains.

TABLE 18E

Domain Analysis of NOV18

gn1|Pfam|pfam00856, SET, SET domain (SEQ ID NO:170)
Length = 125 residues, 100.0% aligned
Score = 123 bits (314), Expect = 6e−29

| | | | |
|---|---|---|---|
| Query: | 4812 | NNVYLARSRIQGLGLYAAKDLEKHTMVIEYIGTIIRNEVANRREKIYE-EQNRGIYMFRI | 4870 |
| | | + + +\| +\| \|\|+\| +\|+ \| ++\|\|+\| \|\| ++ \| \|\|\| \|+ + + \|+\| \| | |
| Sbjct: | 1 | KKLEVFKSPGKGWGLFATEDIPKGEFILEYVGEIITSDEAEEREKAYDTDGAKSSYLFDI | 60 |
| Query: | 4871 | NNEH-VIDATLTGGPARYINHSCAPNCVAEVVTFDKEDKIIIISSRRIPKGEELTYDYQF | 4929 |
| | | +++ \|\|\| \| \|\|+\|\|\|\|\|\|\|\| \| \|++\|+\|+ \|\| \|\|\|\|\|\|\| | |
| Sbjct: | 61 | DSKDLCIDARRKGNLARFINHSCEPNCELVFVEVDGDPRIVIFALRDIKPGEELTIDYGS | 120 |
| Query: | 4930 | DFEDD 4934 | |
| | | \|+\| + | |
| Sbjct: | 121 | DYEGE 125 | |

TABLE 18F

Domain Analysis of NOV18

gn1|Smart|smart00542, FYRC, “FY-rich” domain, C-terminal region (SEQ ID NO:171)
Length = 86 residues, 100.0% aligned
Score = 105 bits (314), Expect = 5e−23

| | | | |
|---|---|---|---|
| Query: | 4655 | EFVIKVIEQGLEDLVFTDASPQAVWNRIIEPVAAMRKEADMLRLFPEYLKGEELFGLTVH | 4714 |
| | | \| ++\| \|\| \|\|+\| \| ++\| \| \| \| +\|+\| \|\| + \|\|++\|\|\|+ | |
| Sbjct: | 1 | LFRVEVESDP--GEVFKGESPEACWEMVLERVQEARIAARLLQLLPEGVSGEDMFGLSSP | 58 |
| Query: | 4715 | AVLRIAESLPGVESCQNYLFRYGRHPLM 4742 | |
| | | \|\|+++ \| \|\|\|\| \| \|\| \|\|\| \| \| + | |
| Sbjct: | 59 | AVVKLIEQLPGVHQCTNYWFRYHRSPEL 86 | |

TABLE 18G

Domain Analysis of NOV18

gnl|Pfam|pfam00628, PHD, PHD-finger (SEQ ID NO:172)
Length = 49 residues, 98.0% aligned
Score = 70.5 bits (171), Expect = 2e−12

| | | | |
|---|---|---|---|
| Query: | 850 | CEVCGQASDPSRLLLCDDCDISYHTYCLDPPLLTVPKGGWKCKWCVSC | 897 |
| | | \| \|\|\|+ \| \|\| \|\| \|\| +\| \|\| \|\|\| \|+\| \| \| \| | |
| Sbjct: | 2 | CAVCGKVDDGGDLLQCDGCDRWFHQACLGPPLEEPPEGKWLCPECTPK | 49 |

TABLE 18H

Domain Analysis of NOV18

gnl|Smart|smart00398, HMG, high mobility group (SEQ ID NO:173)
Length = 71 residues, 76.1% aligned
Score = 48.5 bits (171), Expect = 9e−06

| | | | |
|---|---|---|---|
| Query: | 1441 | VLYANINFPNLKQDYPDWSSR--CKQIMKLWRKVPAADKAPYLQKAKDNRAAHR | 1492 |
| | | +\|++ \| +\| + \|\| + \|++ + \|+ + +\|\|\|\| +\|\|\| + + | |
| Sbjct: | 10 | MLFSQENRKKIKAENPDLKNAEISKKLGERWKLLSEEEKAPYEEKAKKEKERYE | 63 |

The ALL-1 gene is involved in human acute leukemia through chromosome translocations or internal rearrangements. ALL-1 is the human homologue of Drosophila trithorax. The latter is a member of the trithorax group (trx-G) genes which together with the Polycomb group (Pc-G) genes act as positive and negative regulators, respectively, to determine the body structure of Drosophila. ALR, a ALL-1 related protein, which encodes a gigantic 5262 amino acid long protein containing a SET domain, five PHD fingers, potential zinc fingers, and a very long run of glutamines interrupted by hydrophobic residues, mostly leucine. The SET motif, PDH fingers, zinc fingers and two other regions are most similar to domains of ALL-1 and TRX. The first two motifs are also found in other trx-G and Pc-G proteins. The ALR gene was mapped to chromosome band 12q12-13, adjacent to the VDR gene. This region is involved in duplications and translocations associated with cancer. The analysis of ALR expression showed that its approximately 18 kb long mRNA is expressed, like ALL-1, in most adult tissues, including a variety of hematopoietic cells, with the exception of the liver. Whole mount in situ hybridization to early mouse embryos indicates expression in multiple tissues. Based on similarities in structure and expression pattern, ALR is likely to play a similar role to ALL-1 and trx, although its target genes have yet to be identified. (Prasad et al., 1997, Oncogene vol. 15:549-60).

The protein similarity information, expression pattern, and map location for the NOV18 protein and nucleic acid disclosed herein suggest that it may have important structural and/or physiological functions characteristic of the Intracellular family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: cancers such as acute lymphoid leukemia, acute myeloid leukemia, translocation-associated leukemias, and other diseases, disorders and conditions of the like.

The novel nucleic acid encoding the ALR-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV18 protein has multiple hydrophilic regions, each of which can be used as an immunogen.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the celvtissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, or a complement of this aforementioned nucleotide sequence, can be isolated using standard molecular biology techniques and the sequence information provided herein. Using all or a portion of the nucleic acid sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 11, 13, 15, 17, 19, 21, 23, 25, 27, 29, 31, 33, 35, 37, 39, 41,43, 45, 47, 49, 51, 53 and 55, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown NOS: 1, 3, 5, 7, 9, 11, 13, 15, 17, 19, 21, 23, 25, 27, 29, 31, 33, 35, 37, 39 or 41 is one that is sufficiently complementary to the nucleotide sequence shown NOS: 1, 3, 5, 7, 9, 11, 13, 15, 17, 19, 21, 23, 25, 27, 29, 31, 33, 35, 37, 39 or 41 that it can hydrogen bond with little or no mismatches to the nucleotide sequence shown SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and amutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55; or an anti-sense strand nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55; or of a naturally occurring mutant of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53and 55.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55 due to degeneracy of the genetic code and thus encode the same NOVX proteins as that encoded by the nucleotide sequences shown in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55. In another embodiment, an isolated nucleic acid molecule of the invention has a nucleotide sequence encoding a protein having an amino acid sequence shown in SEQ ID NOS: 2, 4, 6, 8, 10, 12, 14, 16, 18, 20, 22, 24, 26, 28, 30, 32, 34, 36, 38, 40,42, 44, 46, 48, 50, 52, 54 and 56.

In addition to the human NOVX nucleotide sequences shown in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56. Preferably, the protein encoded by the nucleic acid molecule is at least about 60% homologous to SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56; more preferably at least about 70% homologous SEQ ID NOS: 2, 4, 6, 8, 10, 12, 14, 16, 18, 20, 22, 24, 26, 28, 30, 32, 34, 36, 38,40, 42, 44, 46, 48, 50, 52, 54 and 56; still more preferably at least about 80% homologous to SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56; even more preferably at least about 90% homologous to SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56; and most preferably at least about 95% homologous to SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56.

An isolated nucleic acid molecule encoding an NOVX protein homologous to the protein of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56 can be created by introducing one or more nucleotide substitutions, additions or deletions into the nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, such that one or more amino acid substitutions, additions or deletions are introduced into the encoded protein.

Mutations can be introduced into SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g. alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof, (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56, or antisense nucleic acids complementary to an NOVX nucleic acid sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Altematively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an (α-anomeric nucleic acid molecule. An a-anomeric nucleic acid molecule forms specific double-stranded hybrids with complementary RNA in which, contrary to the usual P-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55). For example, a derivative of a Tetrahymena L-1 9 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56. The invention also includes a mutant or variant protein any of whose residues may be changed from the corresponding residues shown in SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated afrom cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56. In other embodiments, the NOVX protein is substantially homologous to SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56, and retains the functional activity of the protein of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56, and retains the functional activity of the NOVX proteins of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53and55.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetic s) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′}, and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No.5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No.5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immuinol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g. replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid-sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11 d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55), but more preferably, is a non-human homologue of a human NOVX gene. For example, a mouse homologue of human NOVX gene of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′-and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′-and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (erg., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No.5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′-and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. Bio Technology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g. preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (ize., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

### Identification of NOVX Nucleic Acids

TblastN using CuraGen Corporation's sequence file for polypeptides or homologs was run against the Genomic Daily Files made available by GenBank or from files downloaded from the individual sequencing centers. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The novel NOVX target sequences identified in the present invention were subjected to the exon linking process to confirm the sequence. PCR primers were designed by starting at the most upstream sequence available, for the forward primer, and at the most downstream sequence available for the reverse primer. PCR primer sequences were used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain-amygdala, brain-cerebellum, brain-hippocamppus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

### Example 2

### Identification of Single Nucleotide Polymorphismis in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderbom et al., Determination of Single Nucleotide Polymorphisms by Real-time Pyrophosphate DNA Sequencing. Genome Research. 10 (8)1249-1265, 2000).

### Example 3

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD0.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1X TaqManq® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam squamous,

pl. eff=pl effusion=pleural effusion,

glio=glioma,

astro=astrocytoma, and

neuro=neuroblastoma.

General_screening_panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1 .3D are of the most common cell lines used in the scientific literature.

Panels 4D, 4R, and 4.1D Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-1 3 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5 -10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 μmM Hepes (Gibco) and IL-2 (4 ng/ml). IL-12 (5 ng/ml) and anti-IL4 (1μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, a 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_comprehensive panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-i anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normnal=No apparent disease

Rep22 /Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr 2 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. NOV3 (NOV3a and NOV3b): B7-H2

Expression of the NOV3 gene (CG55790-03 and CG55790-04) was assessed using the primer-probe sets Ag2589, Ag2621 and Ag2915, described in Tables 19, 20 and 21. Results of the RT-PCR runs are shown in Tables 22, 23, 24 and 25.

TABLE 19

Probe Name Ag2589

| | | | | |
|---|---|---|---|---|
| Primers | Sequences | Length | Start Position | SEQ ID NO: |

| | | | | |
|---|---|---|---|---|
| Forward | 5′-gagctcaccttcacgtgtacat-3′ | 22 | 458 | 174 |
| Probe | TET-5′-ctaccccaggcccaacgtgtactg-3′-TAMRA | 24 | 490 | 175 |
| Reverse | 5′-gctgttgtccgtcttattgatc-3′ | 22 | 514 | 176 |

TABLE 20

Probe Name Ag2621

| | | | | |
|---|---|---|---|---|
| Primers | Sequences | Length | Start Position | SEQ ID NO: |

| | | | | |
|---|---|---|---|---|
| Forward | 5′-gagctcaccttcacgtgtacat-3′ | 22 | 458 | 177 |
| Probe | TET-5′-ctaccccaggcccaacgtgtactg-3′-TAMRA | 24 | 490 | 178 |
| Reverse | 5′-gctgttgtccgtcttattgatc-3′ | 22 | 514 | 179 |

TABLE 21

Probe Name Ag2915

| | | | | |
|---|---|---|---|---|
| Primers | Sequences | Length | Start Position | SEQ ID NO: |

| | | | | |
|---|---|---|---|---|
| Forward | 5′-gagctcaccttcacgtgtacat-3′ | 22 | 458 | 180 |
| Probe | TET-5′-ctaccccaggcccaacgtgtactg-3′-TAMRA | 24 | 490 | 181 |
| Reverse | 5′-gctgttgtccgtcttattgatc-3′ | 22 | 514 | 182 |

TABLE 22
CNS_neurodegeneration_v1.0

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag2589, | Ag2621, | Ag2915, | | Ag2589, | Ag2621, | Ag2915, |
| | Run | Run | Run | | Run | Run | Run |
| Tissue Name | 208776915 | 208393684 | 209735956 | Tissue Name | 208776915 | 208393684 | 209735956 |
| AD 1 Hippo | 10.2 | 10.3 | 16.3 | Control (Path) | 1.9 | 1.4 | 1.8 |
| | | | | 3 Temporal | | | |
| | | | | Ctx | | | |
| AD 2 Hippo | 17.2 | 13.9 | 17.4 | Control (Path) | 10.2 | 8.5 | 11.7 |
| | | | | 4 Temporal | | | |
| | | | | Ctx | | | |
| AD 3 Hippo | 6.9 | 4.3 | 5.9 | AD 1 | 9.9 | 6.3 | 11.1 |
| | | | | Occipital Ctx | | | |
| AD 4 Hippo | 5.3 | 3.2 | 6.6 | AD 2 | 0.0 | 0.4 | 0.0 |
| | | | | Occipital Ctx | | | |
| | | | | (Missing) | | | |
| AD 5 Hippo | 33.0 | 27.0 | 40.6 | AD 3 | 4.5 | 6.3 | 5.9 |
| | | | | Occipital Ctx | | | |
| AD 6 Hippo | 60.7 | 49.0 | 59.5 | AD 4 | 14.5 | 10.8 | 14.1 |
| | | | | Occipital Ctx | | | |
| Control 2 | 27.5 | 17.4 | 25.0 | AD 5 | 21.0 | 16.7 | 21.3 |
| Hippo | | | | Occipital Ctx | | | |
| Control 4 | 11.3 | 8.4 | 10.2 | AD 6 | 18.9 | 15.5 | 21.0 |
| Hippo | | | | Occipital Ctx | | | |
| Control (Path) | 4.0 | 3.4 | 4.1 | Control 1 | 3.5 | 2.4 | 2.7 |
| 3 Hippo | | | | Occipital Ctx | | | |
| AD 1 | 15.8 | 12.9 | 15.7 | Control 2 | 24.8 | 25.5 | 36.9 |
| Temporal Ctx | | | | Occipital Ctx | | | |
| AD 2 | 16.8 | 13.9 | 22.5 | Control 3 | 9.0 | 5.8 | 9.0 |
| Temporal Ctx | | | | Occipital Ctx | | | |
| AD 3 | 5.1 | 3.9 | 3.5 | Control 4 | 5.1 | 5.6 | 7.1 |
| Temporal Ctx | | | | Occipital Ctx | | | |
| AD 4 | 13.3 | 12.0 | 18.4 | Control (Path) | 53.6 | 42.3 | 56.6 |
| Temporal Ctx | | | | 1 Occipital | | | |
| | | | | Ctx | | | |
| AD 5 Inf | 66.9 | 59.5 | 84.7 | Control (Path) | 7.8 | 6.3 | 11.2 |
| Temporal Ctx | | | | 2 Occipital | | | |
| | | | | Ctx | | | |
| AD 5 Sup | 35.8 | 30.8 | 43.2 | Control (Path) | 2.3 | 2.7 | 2.2 |
| Temporal Ctx | | | | 3 Occipital | | | |
| | | | | Ctx | | | |
| AD 6 Inf | 100.0 | 100.0 | 100.0 | Control (Path) | 9.9 | 8.1 | 9.9 |
| Temporal Ctx | | | | 4 Occipital | | | |
| | | | | Ctx | | | |
| AD 6 Sup | 50.3 | 35.6 | 52.1 | Control 1 | 7.5 | 6.2 | 6.7 |
| Temporal Ctx | | | | Parietal Ctx | | | |
| Control 1 | 4.0 | 2.4 | 3.8 | Control 2 | 31.4 | 22.2 | 30.1 |
| Temporal Ctx | | | | Parietal Ctx | | | |
| Control 2 | 20.6 | 18.2 | 7.5 | Control 3 | 11.4 | 8.9 | 13.6 |
| Temporal Ctx | | | | Parietal Ctx | | | |
| Control 3 | 8.3 | 5.8 | 7.7 | Control (Path) | 29.1 | 23.5 | 29.1 |
| Temporal Ctx | | | | 1 Parietal Ctx | | | |
| Control 3 | 5.1 | 4.2 | 9.2 | Control (Path) | 11.6 | 9.6 | 17.6 |
| Temporal Ctx | | | | 2 Parietal Ctx | | | |
| Control (Path) | 25.5 | 17.6 | 26.4 | Control (Path) | 2.9 | 1.9 | 1.8 |
| 1 Temporal | | | | 3 Parietal Ctx | | | |
| Ctx | | | | | | | |
| Control (Path) | 13.0 | 11.5 | 12.6 | Control (Path) | 18.6 | 16.3 | 18.8 |
| 2 Temporal Ctx | | | | 4 Parietal Ctx | | | |

TABLE 23
Panel 1.3D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag2589, | Ag2621, | Ag2915, | | Ag2589, | Ag2621, | Ag2915, |
| | Run | Run | Run | | Run | Run | Run |
| Tissue Name | 167660070 | 167644903 | 167646705 | Tissue Name | 167660070 | 167644903 | 167646705 |
| Liver | 23.8 | 17.6 | 22.8 | Kidney (fetal) | 100.0 | 100.0 | 85.9 |
| adenocar- | | | | | | | |
| cinoma | | | | | | | |
| Pancreas | 0.8 | 1.6 | 2.3 | Renal ca. 786- | 27.4 | 28.3 | 33.21 |
| | | | | 0 | | | |
| Pancreatic ca. | 2.6 | 0.5 | 1.0 | Renal ca. | 19.3 | 21.5 | 21.8 |
| CAPAN 2 | | | | A498 | | | |
| Adrenal gland | 3.7 | 3.2 | 1.9 | Renal ca. | 50.0 | 55.9 | 48.6 |
| | | | | RXF 393 | | | |
| Thyroid | 4.2 | 5.3 | 3.3 | Renal ca. | 8.7 | 7.6 | 9.2 |
| | | | | ACHN | | | |
| Salivary gland | 6.3 | 4.0 | 4.2 | Renal ca. UO- | 3.3 | 4.2 | 4.2 |
| | | | | 31 | | | |
| Pituitary gland | 2.0 | 2.0 | 1.4 | Renal ca. TK- | 18.0 | 13.7 | 15.5 |
| | | | | 10 | | | |
| Brain (fetal) | 4.6 | 6.0 | 3.3 | Liver | 5.6 | 4.4 | 8.0 |
| Brain (whole) | 90.8 | 90.8 | 77.4 | Liver (fetal) | 1.9 | 5.6 | 2.6 |
| Brain | 34.9 | 31.4 | 34.4 | Liver ca. | 10.8 | 8.7 | 9.6 |
| (amygdala) | | | | (hepatoblast) | | | |
| | | | | HepG2 | | | |
| Brain | 21.6 | 18.6 | 20.7 | Lung | 12.8 | 13.9 | 17.0 |
| (cerebellum) | | | | | | | |
| Brain | 39.2 | 29.9 | 27.7 | Lung (fetal) | 13.3 | 6.8 | 5.0 |
| (hippocampus) | | | | | | | |
| Brain | 86.5 | 60.7 | 67.8 | Lung ca. | 9.0 | 6.1 | 9.6 |
| (substantia | | | | (small cell) | | | |
| nigra) | | | | LX-1 | | | |
| Brain | 89.5 | 49.7 | 59.0 | Lung ca. | 1.5 | 1.0 | 0.5 |
| (thalamus) | | | | (small cell) | | | |
| | | | | NCI-H69 | | | |
| Cerebral | 46.3 | 33.2 | 38.4 | Lung ca. | 0.0 | 0.0 | 0.5 |
| Cortex | | | | (s. cell var.) | | | |
| | | | | SHP-77 | | | |
| Spinal cord | 29.9 | 20.2 | 31.0 | Lung ca. | 0.6 | 0.0 | 0.7 |
| | | | | (large | | | |
| | | | | cell) NCI- | | | |
| | | | | H460 | | | |
| glio/astro | 18.7 | 13.3 | 18.9 | Lung ca. | 4.0 | 4.0 | 5.7 |
| U87-MG | | | | (non-sm. cell) | | | |
| | | | | A549 | | | |
| glio/astro U- | 0.6 | 1.6 | 1.6 | Lung ca. | 8.4 | 6.6 | 7.8 |
| 118-MG | | | | (non-s. cell) | | | |
| | | | | NCI-H23 | | | |
| astrocytoma | 1.5 | 1.0 | 0.3 | Lung ca. | 1.5 | 2.4 | 3.3 |
| SW1783 | | | | (non-s. cell) | | | |
| | | | | HOP-62 | | | |
| Neuro*; met | 0.0 | 0.0 | 0.0 | Lung ca. | 13.4 | 12.3 | 12.0 |
| SK-N-AS | | | | (non-s. cl) | | | |
| | | | | NCI-H522 | | | |
| astrocytoma | 28.9 | 15.3 | 21.8 | Lung ca. | 5.9 | 5.8 | 4.6 |
| SF-539 | | | | (squam.) SW | | | |
| | | | | 900 | | | |
| astrocytoma | 10.7 | 5.4 | 5.5 | Lung ca. | 0.0 | 0.4 | 1.1 |
| SNB-75 | | | | (squam.) NCI- | | | |
| | | | | H596 | | | |
| glioma SNB- | 2.4 | 1.7 | 3.6 | Mammary | 32.1 | 26.2 | 32.1 |
| 19 | | | | gland | | | |
| glioma U251 | 10.0 | 7.1 | 5.0 | Breast ca.* | 76.3 | 79.0 | 100.0 |
| | | | | (pl. ef) MCF-7 | | | |
| glioma SF-295 | 24.1 | 17.1 | 25.5 | Breast ca.* | 6.2 | 6.7 | 6.5 |
| | | | | (pl. ef) MDA- | | | |
| | | | | MB-231 | | | |
| Heart (fetal) | 29.3 | 24.5 | 31.4 | Breast ca.* | 35.8 | 31.9 | 37.4 |
| | | | | (pl. ef) T47D | | | |
| Heart | 12.8 | 8.5 | 12.2 | Breast ca. BT- | 9.1 | 6.3 | 6.2 |
| | | | | 549 | | | |
| Skeletal | 30.6 | 34.9 | 36.1 | Breast ca. | 2.9 | 4.3 | 6.5 |
| muscle (fetal) | | | | MDA-N | | | |
| Skeletal | 3.1 | 4.4 | 3.2 | Ovary | 5.0 | 6.3 | 6.3 |
| muscle | | | | | | | |
| Bone marrow | 3.3 | 3.5 | 3.6 | Ovarian ca. | 26.2 | 31.6 | 41.2 |
| | | | | OVCAR-3 | | | |
| Thymus | 10.2 | 11.1 | 11.3 | Ovarian ca. | 23.8 | 11.5 | 20.2 |
| | | | | OVCAR-4 | | | |
| Spleen | 11.2 | 10.7 | 15.3 | Ovarian ca. | 20.7 | 17.6 | 14.7 |
| | | | | OVCAR-5 | | | |
| Lymph node | 27.0 | 29.5 | 28.7 | Ovarian ca. | 2.5 | 2.7 | 1.3 |
| | | | | OVCAR-8 | | | |
| Colorectal | 9.5 | 8.1 | 7.5 | Ovarian ca. | 10.7 | 8.1 | 9.9 |
| | | | | IGROV-1 | | | |
| Stomach | 9.5 | 8.2 | 9.7 | Ovarian ca.* | 16.7 | 12.0 | 10.7 |
| | | | | (ascites) SK- | | | |
| | | | | OV-3 | | | |
| Small intestine | 6.8 | 4.6 | 5.7 | Uterus | 2.4 | 4.2 | 4.1 |
| Colon ca. | 7.3 | 6.3 | 7.9 | Placenta | 1.8 | 1.5 | 1.4 |
| SW480 | | | | | | | |
| Colon ca.* | 12.2 | 26.4 | 19.2 | Prostate | 4.6 | 2.9 | 3.8 |
| SW620 | | | | | | | |
| (SW480 | | | | | | | |
| met) | | | | | | | |
| Colon ca. | 5.2 | 4.2 | 4.3 | Prostate ca.* | 19.9 | 17.2 | 19.3 |
| HT29 | | | | (bone | | | |
| | | | | met) PC-3 | | | |
| Colon ca. | 12.2 | 14.7 | 14.2 | Testis | 3.0 | 1.0 | 1.9 |
| HCT-116 | | | | | | | |
| Colon ca. | 30.8 | 28.5 | 29.7 | Melanoma | 0.0 | 0.0 | 0.0 |
| CaCo-2 | | | | Hs688(A).T | | | |
| Colon ca. | 17.3 | 24.3 | 19.3 | Melanoma* | 0.0 | 0.0 | 0.0 |
| tissue | | | | (met) | | | |
| (ODO3866) | | | | Hs688(B).T | | | |
| Colon ca. | 30.8 | 31.9 | 35.8 | Melanoma | 1.0 | 3.6 | 3.8 |
| HCC-2998 | | | | UACC-62 | | | |
| Gastric ca.* | 6.1 | 7.4 | 6.2 | Melanoma | 0.0 | 0.0 | 0.0 |
| (liver met) | | | | M14 | | | |
| NCI-N87 | | | | | | | |
| Bladder | 14.3 | 9.7 | 15.7 | Melanoma | 0.0 | 0.0 | 0.0 |
| | | | | LOX IMVI | | | |
| Trachea | 3.0 | 2.3 | 2.3 | Melanoma* | 1.3 | 2.2 | 2.4 |
| | | | | (met) SK- | | | |
| | | | | MEL-5 | | | |
| Kidney | 24.0 | 23.2 | 21.9 | Adipose | 32.8 | 29.9 | 31.4 |

TABLE 24
Panel 2.2

| | Rel. Exp. (%) | | Rel. Exp. (%) |
|---|---|---|---|
| | Ag2621, Run | | Ag2621, Run |
| Tissue Name | 175063689 | Tissue Name | 175063689 |
| Normal Colon | 6.5 | Kidney Margin (OD04348) | 100.0 |
| Colon cancer (OD06064) | 8.7 | Kidney malignant cancer | 12.3 |
| | | (OD06204B) | |
| Colon Margin (OD06064) | 6.9 | Kidney normal adjacent | 18.9 |
| | | tissue (OD06204E) | |
| Colon cancer (OD06159) | 2.1 | Kidney Cancer (OD04450- | 6.7 |
| | | 01) | |
| Colon Margin (OD06159) | 5.9 | Kidney Margin (OD04450- | 12.9 |
| | | 03) | |
| Colon cancer (OD06297-04) | 3.1 | Kidney Cancer 8120613 | 5.8 |
| Colon Margin (OD06297-015) | 9.7 | Kidney Margin 8120614 | 32.8 |
| CC Gr. 2 ascend colon (ODO3921) | 10.8 | Kidney Cancer 9010320 | 13.8 |
| CC Margin (ODO3921) | 4.1 | Kidney Margin 9010321 | 14.9 |
| Colon cancer metastasis (OD06104) | 6.6 | Kidney Cancer 8120607 | 16.7 |
| Lung Margin (OD06104) | 6.0 | Kidney Margin 8120608 | 10.4 |
| Colon mets to lung (OD04451-01) | 9.9 | Normal Uterus | 9.0 |
| Lung Margin (OD04451-02) | 5.6 | Uterine Cancer 064011 | 4.7 |
| Normal Prostate | 4.7 | Normal Thyroid | 0.7 |
| Prostate Cancer (OD04410) | 2.1 | Thyroid Cancer 064010 | 10.1 |
| Prostate Margin (OD04410) | 4.5 | Thyroid Cancer A302152 | 3.9 |
| Normal Ovary | 2.5 | Thyroid Margin A302153 | 1.2 |
| Ovarian cancer (OD06283-03) | 19.3 | Normal Breast | 10.9 |
| Ovarian Margin (OD06283-07) | 7.6 | Breast Cancer (OD04566) | 9.5 |
| Ovarian Cancer 064008 | 5.6 | Breast Cancer 1024 | 28.3 |
| Ovarian cancer (OD06145) | 6.5 | Breast Cancer (OD04590- | 32.3 |
| | | 01) | |
| Ovarian Margin (OD06145) | 11.7 | Breast Cancer Mets | 13.6 |
| | | (OD04590-03) | |
| Ovarian cancer (OD06455-03) | 4.1 | Breast Cancer Metastasis | 12.9 |
| | | (OD04655-05) | |
| Ovarian Margin (OD06455-07) | 5.6 | Breast Cancer 064006 | 12.9 |
| Normal Lung | 14.6 | Breast Cancer 9100266 | 5.8 |
| Invasive poor diff. lung adeno | 3.8 | Breast Margin 9100265 | 7.8 |
| (ODO4945-01 | | | |
| Lung Margin (ODO4945-03) | 6.3 | Breast Cancer A209073 | 4.7 |
| Lung Malignant Cancer (OD03126) | 4.2 | Breast Margin A2090734 | 23.3 |
| Lung Margin (OD03126) | 6.7 | Breast cancer (OD06083) | 23.5 |
| Lung Cancer (OD05014A) | 5.9 | Breast cancer node | 15.8 |
| | | metastasis (OD06083) | |
| Lung Margin (OD05014B) | 8.5 | Normal Liver | 23.2 |
| Lung cancer (OD06081) | 5.5 | Liver Cancer 1026 | 5.6 |
| Lung Margin (OD06081) | 3.5 | Liver Cancer 1025 | 13.6 |
| Lung Cancer (OD04237-01) | 3.0 | Liver Cancer 6004-T | 19.1 |
| Lung Margin (OD04237-02) | 17.4 | Liver Tissue 6004-N | 1.4 |
| Ocular Melanoma Metastasis | 3.2 | Liver Cancer 6005-T | 19.2 |
| Ocular Melanoma Margin (Liver) | 9.7 | Liver Tissue 6005-N | 18.3 |
| Melanoma Metastasis | 1.4 | Liver Cancer 064003 | 2.2 |
| Melanoma Margin (Lung) | 5.3 | Normal Bladder | 16.2 |
| Normal Kidney | 10.6 | Bladder Cancer 1023 | 8.2 |
| Kidney Ca, Nuclear grade 2 | 45.7 | Bladder Cancer A302173 | 27.4 |
| (OD04338) | | | |
| Kidney Margin (OD04338) | 10.6 | Normal Stomach | 18.4 |
| Kidney Ca Nuclear grade 1/2 | 33.2 | Gastric Cancer 9060397 | 17.0 |
| (OD04339) | | | |
| Kidney Margin (OD04339) | 23.0 | Stomach Margin 9060396 | 4.5 |
| Kidney Ca, Clear cell type | 47.3 | Gastric Cancer 9060395 | 5.7 |
| (OD04340) | | | |
| Kidney Margin (OD04340) | 14.7 | Stomach Margin 9060394 | 13.6 |
| Kidney Ca, Nuclear grade 3 | 6.0 | Gastric Cancer 064005 | 11.3 |
| (OD04348) | | | |

TABLE 25
Panel 4D

| | Rel. Exp. (%) | Rel. Exp. (%) | Rel. Exp. (%) | Rel. Exp. (%) |
|---|---|---|---|---|
| | Ag2589, Run | Ag2589, Run | Ag2621, Run | Ag2915, Run |
| Tissue Name | 164289988 | 164347841 | 164299478 | 164403111 |
| Secondary Th1 act | 1.7 | 1.7 | 2.1 | 2.0 |
| Secondary Th2 act | 2.4 | 2.4 | 2.0 | 1.7 |
| Secondary Tr1 act | 2.3 | 2.3 | 3.0 | 2.1 |
| Secondary Th1 rest | 0.4 | 0.4 | 0.5 | 0.6 |
| Secondary Th2 rest | 0.6 | 0.6 | 1.1 | 0.5 |
| Secondary Tr1 rest | 1.1 | 1.1 | 1.8 | 0.9 |
| Primary Th1 act | 2.0 | 2.0 | 3.1 | 2.0 |
| Primary Th2 act | 3.0 | 3.0 | 4.6 | 3.8 |
| Primary Tr1 act | 3.1 | 3.1 | 6.2 | 4.2 |
| Primary Th1 rest | 2.8 | 2.8 | 6.0 | 4.0 |
| Primary Th2 rest | 1.6 | 1.6 | 3.8 | 1.8 |
| Primary Tr1 rest | 2.0 | 2.0 | 2.6 | 2.4 |
| CD45RA CD4 lymphocyte act | 1.8 | 1.8 | 1.7 | 1.7 |
| CD45RO CD4 lymphocyte act | 3.4 | 3.4 | 1.9 | 2.2 |
| CD8 lymphocyte act | 1.1 | 1.1 | 0.8 | 1.4 |
| Secondary CD8 lymphocyte rest | 1.8 | 1.8 | 2.2 | 1.9 |
| Secondary CD8 lymphocyte act | 1.3 | 1.3 | 0.8 | 1.2 |
| CD4 lymphocyte none | 1.2 | 1.2 | 1.6 | 1.2 |
| 2ry Th1/Th2/Tr1_anti-CD95 CH11 | 1.9 | 1.9 | 1.9 | 1.1 |
| LAK cells rest | 12.2 | 12.2 | 8.5 | 6.7 |
| LAK cells IL-2 | 1.4 | 1.4 | 1.1 | 0.7 |
| LAK cells IL-2 + IL-12 | 1.7 | 1.7 | 2.3 | 1.2 |
| LAK cells IL-2 + IFN gamma | 2.7 | 2.7 | 3.1 | 3.0 |
| LAK cells IL-2 + IL-18 | 2.6 | 2.6 | 3.2 | 2.2 |
| LAK cells PMA/ionomycin | 4.1 | 4.1 | 3.6 | 3.9 |
| NK Cells IL-2 rest | 0.6 | 0.6 | 0.8 | 0.6 |
| Two Way MLR 3 day | 9.2 | 9.2 | 9.5 | 8.8 |
| Two Way MLR 5 day | 3.9 | 3.9 | 4.4 | 2.5 |
| Two Way MLR 7 day | 1.8 | 1.8 | 1.6 | 1.1 |
| PBMC rest | 6.8 | 6.8 | 5.7 | 4.6 |
| PBMC PWM | 4.3 | 4.3 | 5.8 | 5.4 |
| PBMC PHA-L | 2.2 | 2.2 | 2.0 | 2.5 |
| Ramos (B cell) none | 13.8 | 13.8 | 19.2 | 15.2 |
| Ramos (B cell) ionomycin | 22.7 | 22.7 | 30.6 | 26.2 |
| B lymphocytes PWM | 10.9 | 10.9 | 18.7 | 11.3 |
| B lymphocytes CD40L and IL-4 | 14.6 | 14.6 | 26.8 | 20.2 |
| EOL-1 dbcAMP | 23.7 | 23.7 | 26.8 | 25.3 |
| EOL-1 dbcAMP PMA/ionomycin | 100.0 | 100.0 | 100.0 | 100.0 |
| Dendritic cells none | 12.9 | 12.9 | 9.9 | 8.6 |
| Dendritic cells LPS | 19.2 | 19.2 | 23.3 | 17.3 |
| Dendritic cells anti-CD40 | 16.4 | 16.4 | 17.1 | 11.7 |
| Monocytes rest | 13.6 | 3.6 | 4.5 | 4.0 |
| Monocytes LPS | 11.5 | 11.5 | 12.2 | 11.2 |
| Macrophages rest | 6.2 | 6.2 | 10.5 | 7.9 |
| Macrophages LPS | 12.0 | 12.0 | 15.7 | 13.6 |
| HUVEC none | 1.8 | 1.8 | 2.3 | 1.1 |
| HUVEC starved | 3.1 | 3.1 | 4.3 | 3.9 |
| HUVEC IL-1beta | 6.2 | 6.2 | 9.6 | 7.6 |
| HUVEC IFN gamma | 2.4 | 2.4 | 1.8 | 2.0 |
| HUVEC TNF alpha + IFN gamma | 22.1 | 22.1 | 26.1 | 20.7 |
| HUVEC TNF alpha + IL4 | 28.7 | 28.7 | 20.2 | 19.2 |
| HUVEC IL-11 | 1.8 | 1.8 | 1.1 | 1.3 |
| Lung Microvascular EC none | 2.0 | 2.0 | 2.8 | 2.2 |
| Lung Microvascular EC TNFalpha + | 54.3 | 54.3 | 56.6 | 48.3 |
| IL-1beta | | | | |

| | | | | |
|---|---|---|---|---|
| Microvascular Dermal EC none | 1.5 | 1.5 | 1.0 | 1.3 |
| Microsvasular Dermal EC TNFalpha + | 47.3 | 47.3 | 61.6 | 48.6 |
| IL-1beta | | | | |
| Bronchial epithelium TNFalpha + | 3.2 | 3.2 | 4.7 | 3.1 |
| IL1beta | | | | |
| Small airway epithelium none | 0.4 | 0.4 | 0.9 | 0.8 |
| Small airway epithelium TNFalpha + | 3.7 | 3.7 | 5.4 | 5.6 |
| IL-1beta | | | | |
| Coronery artery SMC rest | 0.3 | 0.3 | 0.5 | 0.1 |
| Coronery artery SMC TNFalpha + IL- | 0.8 | 0.8 | 0.8 | 1.0 |
| 1beta | | | | |
| Astrocytes rest | 0.4 | 0.4 | 0.8 | 0.8 |
| Astrocytes TNFalpha + IL-1beta | 26.2 | 26.2 | 27.9 | 22.8 |
| KU-812 (Basophil) rest | 0.7 | 0.7 | 0.4 | 0.3 |
| KU-812 (Basophil) PMA/ionomycin | 1.9 | 1.9 | 2.4 | 1.9 |
| CCD1106 (Keratinocytes) none | 1.0 | 1.0 | 1.4 | 1.0 |
| CCD1106 (Keratinocytes) TNFalpha + | 2.8 | 2.8 | 3.7 | 2.3 |
| IL-1beta | | | | |
| Liver cirrhosis | 1.0 | 1.0 | 0.9 | 0.9 |
| Lupus kidney | 1.9 | 1.9 | 1.9 | 1.7 |
| NCI-H292 none | 2.6 | 2.6 | 3.3 | 3.2 |
| NCI-H292 IL-4 | 2.2 | 2.2 | 2.1 | 2.6 |
| NCI-H292 IL-9 | 3.2 | 3.2 | 2.1 | 2.8 |
| NCI-H292 IL-13 | 2.2 | 2.2 | 1.6 | 1.2 |
| NCI-H292 IFN gamma | 5.0 | 5.0 | 4.5 | 4.2 |
| HPAEC none | 1.5 | 1.5 | 0.8 | 0.9 |
| HPAEC TNF alpha + IL-1 beta | 69.3 | 69.3 | 89.5 | 70.2 |
| Lung fibroblast none | 0.1 | 0.1 | 0.0 | 0.1 |
| Lung fibroblast TNF alpha + | 0.6 | 0.6 | 0.5 | 0.5 |
| IL-1 beta | | | | |
| Lung fibroblast IL-4 | 0.1 | 0.1 | 0.1 | 0.0 |
| Lung fibroblast IL-9 | 0.0 | 0.0 | 0.1 | 0.0 |
| Lung fibroblast IL-13 | 0.0 | 0.0 | 0.0 | 0.0 |
| Lung fibroblast IFN gamma | 0.3 | 0.3 | 0.1 | 0.2 |
| Dermal fibroblast CCD1070 rest | 0.7 | 0.7 | 0.6 | 0.4 |
| Dermal fibroblast CCD1070 TNF | 3.2 | 3.2 | 4.2 | 2.9 |
| alpha | | | | |
| Dermal fibroblast CCD1070 IL-1 beta | 0.7 | 0.7 | 0.4 | 0.5 |
| Dermal fibroblast IFN gamma | 0.3 | 0.3 | 0.5 | 0.4 |
| Dermal fibroblast IL-4 | 0.6 | 0.6 | 0.4 | 0.4 |
| IBD Colitis 2 | 1.0 | 1.0 | 1.4 | 1.3 |
| IBD Crohn's | 0.4 | 0.4 | 0.5 | 0.3 |
| Colon | 5.8 | 5.8 | 9.6 | 5.3 |
| Lung | 3.2 | 3.2 | 7.2 | 4.5 |
| Thymus | 11.0 | 11.0 | 14.2 | 12.9 |
| Kidney | 4.5 | 4.5 | 7.1 | 7.0 |

CNS_neurodegeneration_v1.0 Summary: Ag2589/Ag2621/Ag2915 Multiple experiments with the same probe and primer set produce results are in excellent agreement. In all cases, the expression of the NOV3a gene is up-regulated in the temporal cortex of Alzheimer's disease patients when compared to non-demented controls. This difference is apparent when data are analyzed via ANCOVA, using overall RNA quality and/or quantity as a covariate. The up-regulation of this gene is most apparent in the variant detected by Ag1845. The temporal cortex is a region that shows degeneration at the mid-stages of this disease. Thus, it is likely that the phenomenon of neurodegeneration was captured in this region, as opposed to the hippocampus and entorhinal cortex where a large number of neurons are already lost by the time of death in AD. Furthermore, in the occipital cortex (where neurodegeneration does not occur in Alzheimer's) this gene is not found to be up-regulated in the same patients. Taken together, these data suggest that this gene is at least a marker of Alzheimer's-like neurodegeneration, and is probably involved in the process of neurodegeneration.

Furthermore, this gene is a form of B7 protein (B7-H2B), which plays a role in inflammation. Neuroinflammation has been implicated in AD, to the extent that long-term usage of anti-inflammatory agents has been correlated with a reduced incidence of Alzheimer's in retrospective studies. This gene therefore represents an excellent drug target for the treatment of Alzheimer's disease, and any other neuroinflammatory condition.

Panel 1.3D Summary: Ag2589/Ag2621/Ag2915 Multiple experiments with the same probe and primer set produce results that are in excellent agreement. Highest expression of the NOV3a gene is seen in the brain, fetal kidney, and a breast cancer cell line.

Expression in the CNS panel confirms the expression of this gene in the CNS. Please see panel CNS_Neurodegeneration for a discussion of utility of this gene in the central nervous system.

Higher levels of expression are also consistently seen in fetal skeletal muscle (CTs=29-30), when compared to expression in adult skeletal muscle (CTs=33-35). Thus, expression of the NOV3a gene could be used to differentiate between the adult and fetal sources of this tissue.

The NOV3a gene product is also moderately expressed in pancreas, adrenal, thyroid, pituitary, adult and fetal liver, adult and fetal heart, and adipose. Based on its expression profile in metabolic tissues, this gene product may be useful in the diagnosis and/or treatment of metabolic disease, including obesity and diabetes.

Panel 2.2 Summary: Ag2621 The expression of the NOV3a gene appears to be highest in a sample derived from a normal kidney margin (CT=29.1). In addition, there appears to be substantial expression associated with several kidney cancer samples. Thus, the expression of this gene could be used to distinguish this normal kidney sample from others in the panel. Moreover, therapeutic modulation of this gene, through the use of small molecule drugs, protein therapeutics or antibodies might be beneficial for the treatment of kidney cancer.

Panel 4D Summary: Ag2589/Ag2621/Ag2915 The NOV3a transcript is highly expressed in activated EOL cells, activated lung and dermal microvascular endothelium, activated human pulmonary aortic endothelial cells and in TNFalpha activated human umbilical vein endothelial cells. NOV3a encodes B7-H2, which has been shown to be important in antigen presentation. It is a ligand for ICOS and serves as a costimulatory molecule (Ref. 1-2). Therefore, monoclonal antibody therapeutics designed with the NOV3a protein product may reduce or inhibit antigen presentation and be important in the treatment of diseases such as asthma in which T cells are chronically stimulated.

References:

Ling V, Wu P W, Finnerty H F, Bean K M, Spaulding V, Fouser L A, Leonard J P, Hunter S E, Zollner R, Thomas J L, Miyashiro J S, Jacobs K A, Collins M. Cutting edge: identification of GL50, a novel B7-like protein that functionally binds to ICOS receptor. J Immunol Feb. 15, 2000;164(4):1653-7

By the genetic selection of mouse cDNAs encoding secreted proteins, a B7-like cDNA clone termed mouse GL50 (mGL50) was isolated encoding a 322-aa polypeptide identical with B7h. Isolation of the human ortholog of this cDNA (hGL50) revealed a coding sequence of 309 aa residues with 42% sequence identity with mGL50. Northern analysis indicated GL50 to be present in many tissues including lymphoid, embryonic yolk sac, and fetal liver samples. Of the CD28, CTLA4, and ICOS fusion constructs tested, flow cytometric analysis demonstrated only mouse ICOS-IgG binding to mGL50 cell transfectants. Subsequent phenotyping demonstrated high levels of ICOS ligand staining on splenic CD19+ B cells and low levels on CD3+ T cells. These results indicate that GL50 is a specific ligand for the ICOS receptor and suggest that the GL50-ICOS interaction functions in lymphocyte costimulation.

Wang S, Zhu G, Chapoval A I, Dong H, Tamada K, Ni J, Chen L. Costimulation of T cells by B7-H2, a B7-like molecule that binds ICOS. Blood Oct. 15, 2000 ;96(8):2808-13

This report describes a new human B7-like gene designated B7-H2. Cell surface expression of B7-H2 protein is detected in monocyte-derived immature dendritic cells. Soluble B7-H2 and immunoglobulin (Ig) fusion protein, B7-H2Ig, binds activated but not resting T cells and the binding is abrogated by inducible costimulator Ig (ICOSIg), but not CTLA4Ig. In addition, ICOSIg stains Chinese hamster ovary cells transfected with B7-H2 gene. By suboptimal cross-linking of CD3, costimulation of T-cell proliferation by B7-H2Ig is dose-dependent and correlates with secretion of interleukin (IL)-2, whereas optimal CD3 ligation preferentially stimulates IL-10 production. The results indicate that B7-H2 is a putative ligand for the ICOS T-cell molecule. (Blood. 2000;96:2808-2813)

PMID: 11023515

B. NOV4a: B7-H1

Expression of the NOV4a gene (CG56110-01) was assessed using the primer-probe set Ag1544, described in Table 26. Results of the RTQ-PCR runs are shown in Tables 27, 28, 29 and 30.

TABLE 26

Probe Name Ag1544

| | | | | |
|---|---|---|---|---|
| Primers | Sequences | Length | Start Position | SEQ ID NO: |

| | | | | |
|---|---|---|---|---|
| Forward | 5′-tctggacaagcagtgaccat-3′ | 20 | 497 | 183 |
| Probe | TET-5′-accaccaccaattccaagagagagga-3′-TAMRA | 26 | 538 | 184 |
| Reverse | 5′-ttctcagtgtgctggtcaca-3′ | 20 | 576 | 185 |

TABLE 27
Panel 1.2

| | Rel. Exp. (%) | | Rel. Exp. (%) |
|---|---|---|---|
| | Ag1544, Run | | Ag1544, Run |
| Tissue Name | 142185523 | Tissue Name | 142185523 |
| Endothelial cells | 21.0 | Renal ca. 786-0 | 6.0 |
| Heart (Fetal) | 4.8 | Renal ca. A498 | 16.6 |
| Pancreas | 0.0 | Renal ca. RXF 393 | 7.4 |
| Pancreatic ca. CAPAN2 | 1.4 | Renal ca. ACHN | 5.9 |
| Adrenal Gland | 9.6 | Renal ca. UO-31 | 17.0 |
| Thyroid | 0.0 | Renal ca. TK-10 | 0.9 |
| Salivary gland | 13.0 | Liver | 4.9 |
| Pituitary gland | 0.0 | Liver (fetal) | 6.1 |
| Brain (fetal) | 0.0 | Liver ca. (hepatoblast) HepG2 | 0.0 |
| Brain (whole) | 0.0 | Lung | 3.5 |
| Brain (amygdala) | 0.0 | Lung (fetal) | 0.4 |
| Brain (cerebellum) | 0.0 | Lung ca. (small cell) LX-1 | 2.3 |
| Brain (hippocampus) | 1.6 | Lung ca. (small cell) NCI-H69 | 0.0 |
| Brain (thalamus) | 0.7 | Lung ca. (s. cell var.) SHP-77 | 0.0 |
| Cerebral Cortex | 9.5 | Lung ca. (large cell) NCI-H460 | 24.3 |
| Spinal cord | 0.0 | Lung ca. (non-sm. cell) A549 | 0.0 |
| glio/astro U87-MG | 28.9 | Lung ca. (non-s. cell) NCI-H23 | 0.0 |
| glio/astro U-118-MG | 1.0 | Lung ca. (non-s. cell) HOP-62 | 40.9 |
| astrocytoma SW1783 | 1.2 | Lung ca. (non-s. cl) NCI-H522 | 0.0 |
| Neuro*; met SK-N-AS | 4.5 | Lung ca. (squam.) SW 900 | 88.9 |
| astrocytoma SF-539 | 1.1 | Lung ca. (squam.) NCI-H596 | 0.0 |
| astrocytoma SNB-75 | 0.0 | Mammary gland | 0.0 |
| glioma SNB-19 | 13.4 | Breast ca.* (pl. ef) MCF-7 | 0.0 |
| glioma U251 | 5.1 | Breast ca.* (pl. ef) MDA-MB-231 | 14.0 |
| glioma SF-295 | 10.4 | Breast ca.* (pl. ef) T47D | 0.0 |
| Heart | 100.0 | Breast ca. BT-549 | 4.4 |
| Skeletal Muscle | 42.6 | Breast ca. MDA-N | 9.3 |
| Bone marrow | 0.0 | Ovary | 0.1 |
| Thymus | 0.5 | Ovarian ca. OVCAR-3 | 0.0 |
| Spleen | 12.2 | Ovarian ca. OVCAR-4 | 6.1 |
| Lymph node | 0.0 | Ovarian ca. OVCAR-5 | 18.9 |
| Colorectal Tissue | 1.7 | Ovarian ca. OVCAR-8 | 4.5 |
| Stomach | 0.0 | Ovarian ca. IGROV-1 | 0.0 |
| Small intestine | 9.9 | Ovarian ca. (ascites) SK-OV-3 | 0.1 |
| Colon ca. SW480 | 0.0 | Uterus | 0.0 |
| Colon ca.* SW620 (SW480 met) | 0.0 | Placenta | 29.3 |
| Colon ca. HT29 | 0.0 | Prostate | 3.7 |
| Colon ca. HCT-116 | 2.0 | Prostate ca.* (bone met) PC-3 | 5.3 |
| Colon ca. CaCo-2 | 0.0 | Testis | 0.0 |
| Colon ca. Tissue (ODO3866) | 0.0 | Melanoma Hs688(A).T | 0.0 |
| Colon ca. HCC-2998 | 10.5 | Melanoma* (met) Hs688(B).T | 0.0 |
| Gastric ca.* (liver met) NCI-N87 | 57.0 | Melanoma UACC-62 | 3.5 |
| Bladder | 23.7 | Melanoma M14 | 0.0 |
| Trachea | 0.0 | Melanoma LOX IMVI | 23.0 |
| Kidney | 18.4 | Melanoma* (met) SK-MEL-5 | 11.5 |
| Kidney (fetal) | 0.0 | | |

TABLE 28
Panel 1.3D

| | Rel. Exp. (%) | | Rel. Exp. (%) |
|---|---|---|---|
| | Ag1544, Run | | Ag1544, Run |
| Tissue Name | 146169090 | Tissue Name | 146169090 |
| Liver adenocarcinoma | 3.6 | Kidney (fetal) | 5.5 |
| Pancreas | 2.9 | Renal ca. 786-0 | 10.0 |
| Pancreatic ca. CAPAN 2 | 5.0 | Renal ca. A498 | 17.8 |
| Adrenal gland | 2.4 | Renal ca. RXF 393 | 11.0 |
| Thyroid | 2.3 | Renal ca. ACHN | 6.9 |
| Salivary gland | 1.7 | Renal ca. UO-31 | 14.4 |
| Pituitary gland | 6.3 | Renal ca. TK-10 | 0.2 |
| Brain (fetal) | 1.8 | Liver | 2.2 |
| Brain (whole) | 3.5 | Liver (fetal) | 5.8 |
| Brain (amygdala) | 2.8 | Liver ca. (hepatoblast) HepG2 | 0.1 |
| Brain (cerebellum) | 1.7 | Lung | 27.9 |
| Brain (hippocampus) | 4.5 | Lung (fetal) | 14.0 |
| Brain (substantia nigra) | 2.7 | Lung ca. (small cell) LX-1 | 3.4 |
| Brain (thalamus) | 4.4 | Lung ca. (small cell) NCI-H69 | 0.2 |
| Cerebral Cortex | 10.4 | Lung ca. (s. cell var.) SHP-77 | 3.0 |
| Spinal cord | 2.0 | Lung ca. (large cell) NCI-H460 | 9.7 |
| glio/astro U87-MG | 25.5 | Lung ca. (non-sm. cell) A549 | 0.8 |
| glio/astro U-118-MG | 8.2 | Lung ca. (non-s. cell) NCI-H23 | 1.3 |
| astrocytoma SW1783 | 7.9 | Lung ca. (non-s. cell) HOP-62 | 19.3 |
| Neuro*; met SK-N-AS | 9.2 | Lung ca. (non-s. cl) NCI-H522 | 0.0 |
| astrocytoma SF-539 | 6.3 | Lung ca. (squam.) SW 900 | 33.2 |
| astrocytoma SNB-75 | 37.9 | Lung ca. (squam.) NCI-H596 | 0.0 |
| glioma SNB-19 | 12.3 | Mammary gland | 5.6 |
| glioma U251 | 14.7 | Breast ca.* (pl. ef) MCF-7 | 0.0 |
| glioma SF-295 | 6.0 | Breast ca.* (pl. ef) MDA-MB- | 62.0 |
| | | 231 | |
| Heart (fetal) | 9.0 | Breast ca.* (pl. ef) T47D | 0.0 |
| Heart | 5.4 | Breast ca. BT-549 | 11.1 |
| Skeletal muscle (fetal) | 6.2 | Breast ca. MDA-N | 5.9 |
| Skeletal muscle | 1.9 | Ovary | 3.5 |
| Bone marrow | 3.2 | Ovarian ca. OVCAR-3 | 1.7 |
| Thymus | 13.4 | Ovarian ca. OVCAR-4 | 0.1 |
| Spleen | 14.0 | Ovarian ca. OVCAR-5 | 5.9 |
| Lymph node | 27.0 | Ovarian ca. OVCAR-8 | 3.6 |
| Colorectal | 1.5 | Ovarian ca. IGROV-1 | 0.0 |
| Stomach | 3.4 | Ovarian ca.* (ascites) SK-OV- | 0.6 |
| | | 3 | |
| Small intestine | 2.7 | Uterus | 2.7 |
| Colon ca. SW480 | 2.2 | Placenta | 59.0 |
| Colon ca* SW620 (SW480 met) | 0.8 | Prostate | 1.7 |
| Colon ca. HT29 | 0.6 | Prostate ca.* (bone met) PC-3 | 3.1 |
| Colon ca. HCT-116 | 2.6 | Testis | 3.7 |
| Colon ca. CaCo-2 | 0.6 | Melanoma Hs688(A).T | 15.1 |
| Colon ca. tissue (ODO3866) | 6.6 | Melanoma* (met) Hs688(B).T | 9.7 |
| Colon ca. HCC-2998 | 4.5 | Melanoma UACC-62 | 1.4 |
| Gastric ca.* (liver met) NCI-N87 | 100.0 | Melanoma M14 | 0.1 |
| Bladder | 6.7 | Melanoma LOX IMVI | 17.8 |
| Trachea | 10.9 | Melanoma* (met) SK-MEL-5 | 7.4 |
| Kidney | 0.8 | Adipose | 5.1 |

TABLE 29
Panel 2D

| | Rel. | Rel. | | Rel. | Rel. |
|---|---|---|---|---|---|
| | Exp. (%) | Exp. (%) | | Exp. (%) | Exp. (%) |
| | Ag1544, | Ag1544, | | Ag1544, | Ag1544, |
| | Run | Run | | Run | Run |
| Tissue Name | 145030196 | 145361330 | Tissue Name | 145030196 | 145361330 |
| Normal Colon | 15.7 | 2.5 | Kidney Margin | 0.7 | 0.8 |
| | | | 8120608 | | |
| CC Well to Mod Diff | 5.1 | 0.8 | Kidney Cancer | 1.2 | 1.7 |
| (ODO3866) | | | 8120613 | | |
| CC Margin (ODO3866) | 3.8 | 0.5 | Kidney Margin | 0.0 | 0.3 |
| | | | 8120614 | | |
| CC Gr. 2 rectosigmoid | 3.3 | 1.2 | Kidney Cancer | 4.7 | 1.2 |
| (ODO3868) | | | 9010320 | | |
| CC Margin (ODO3868) | 0.0 | 0.1 | Kidney Margin | 2.1 | 0.7 |
| | | | 9010321 | | |
| CC Mod Diff (ODO3920) | 2.6 | 1.8 | Normal Uterus | 2.7 | 1.6 |
| CC Margin (ODO3920) | 3.0 | 2.2 | Uterus Cancer 064011 | 6.0 | 6.3 |
| CC Gr. 2 ascend colon | 6.7 | 4.0 | Normal Thyroid | 7.3 | 4.5 |
| (ODO3921) | | | | | |
| CC Margin (ODO3921) | 1.6 | 0.9 | Thyroid Cancer | 36.3 | 29.9 |
| | | | 064010 | | |
| CC from Partial | 6.0 | 5.4 | Thyroid Cancer | 11.6 | 13.8 |
| Hepatectomy (ODO4309) | | | A302152 | | |
| Mets | | | | | |
| Liver Margin (ODO4309) | 9.8 | 4.7 | Thyroid Margin | 15.7 | 9.9 |
| | | | A302153 | | |
| Colon mets to lung | 7.3 | 7.2 | Normal Breast | 5.2 | 3.9 |
| (OD04451-01) | | | | | |
| Lung Margin (OD04451- | 13.6 | 2.4 | Breast Cancer | 5.0 | 3.3 |
| 02) | | | (OD04566) | | |
| Normal Prostate 6546-1 | 3.5 | 2.2 | Breast Cancer | 8.6 | 2.3 |
| | | | (OD04590-01) | | |
| Prostate Cancer | 5.1 | 3.7 | Breast Cancer Mets | 6.7 | 7.2 |
| (OD04410) | | | (OD04590-03) | | |
| Prostate Margin | 4.5 | 8.0 | Breast Cancer | 6.6 | 5.6 |
| (OD04410) | | | Metastasis (OD04655- | | |
| | | | 05) | | |
| Prostate Cancer | 6.3 | 7.1 | Breast Cancer 064006 | 15.3 | 11.2 |
| (OD04720-01) | | | | | |
| Prostate Margin | 8.1 | 11.5 | Breast Cancer 1024 | 1.9 | 0.9 |
| (OD04720-02) | | | | | |
| Normal Lung 061010 | 34.2 | 49.0 | Breast Cancer 9100266 | 3.1 | 3.5 |
| Lung Met to Muscle | 25.7 | 58.2 | Breast Margin 9100265 | 2.4 | 1.9 |
| (ODO4286) | | | | | |
| Muscle Margin | 5.7 | 4.9 | Breast Cancer | 5.1 | 7.2 |
| (ODO4286) | | | A209073 | | |
| Lung Malignant Cancer | 13.9 | 25.7 | Breast Margin | 5.4 | 1.2 |
| (OD03126) | | | A2090734 | | |
| Lung Margin (OD03126) | 39.0 | 32.3 | Normal Liver | 6.0 | 4.3 |
| Lung Cancer (OD04404) | 27.4 | 15.1 | Liver Cancer 064003 | 4.7 | 1.1 |
| Lung Margin (OD04404) | 25.0 | 4.9 | Liver Cancer 1025 | 1.8 | 1.5 |
| Lung Cancer (OD04565) | 6.2 | 5.8 | Liver Cancer 1026 | 0.0 | 0.5 |
| Lung Margin (OD04565) | 12.9 | 17.1 | Liver Cancer 6004-T | 4.3 | 1.1 |
| Lung Cancer (OD04237- | 27.5 | 17.6 | Liver Tissue 6004-N | 4.2 | 1.3 |
| 01) | | | | | |
| Lung Margin (OD04237- | 100.0 | 28.9 | Liver Cancer 6005-T | 0.5 | 0.4 |
| 02) | | | | | |
| Ocular Mel Met to Liver | 3.3 | 3.9 | Liver Tissue 6005-N | 1.6 | 1.0 |
| (ODO4310) | | | | | |
| Liver Margin (ODO4310) | 5.0 | 5.6 | Normal Bladder | 11.3 | 12.7 |
| Melanoma Mets to Lung | 5.5 | 3.7 | Bladder Cancer 1023 | 1.8 | 1.3 |
| (OD04321) | | | | | |
| Lung Margin (OD04321) | 35.8 | 32.1 | Bladder Cancer | 50.0 | 16.7 |
| | | | A302173 | | |
| Normal Kidney | 12.1 | 4.8 | Bladder Cancer | 96.6 | 100.0 |
| | | | (OD04718-01) | | |
| Kidney Ca, Nuclear grade | 12.7 | 11.9 | Bladder Normal | 11.1 | 4.6 |
| 2 (OD04338) | | | Adjacent (OD04718- | | |
| | | | 03) | | |
| Kidney Margin | 6.7 | 2.6 | Normal Ovary | 0.3 | 0.4 |
| (OD04338) | | | | | |
| Kidney Ca Nuclear grade | 14.7 | 9.2 | Ovarian Cancer | 7.4 | 3.4 |
| 1/2 (OD04339) | | | 064008 | | |
| Kidney Margin | 6.6 | 3.2 | Ovarian Cancer | 23.0 | 0.4 |
| (OD04339) | | | (OD04768-07) | | |
| Kidney Ca, Clear cell type | 7.3 | 9.4 | Ovary Margin | 5.5 | 2.0 |
| (OD04340) | | | (OD04768-08) | | |
| Kidney Margin | 8.2 | 5.1 | Normal Stomach | 5.2 | 2.1 |
| (OD04340) | | | | | |
| Kidney Ca, Nuclear grade | 6.7 | 8.1 | Gastric Cancer | 1.8 | 0.4 |
| 3 (OD04348) | | | 9060358 | | |
| Kidney Margin | 17.9 | 18.9 | Stomach Margin | 2.3 | 1.3 |
| (OD04348) | | | 9060359 | | |
| Kidney Cancer (OD04622- | 7.5 | 15.9 | Gastric Cancer | 5.9 | 2.0 |
| 01) | | | 9060395 | | |
| Kidney Margin | 1.9 | 0.7 | Stomach Margin | 5.0 | 1.9 |
| (OD04622-03) | | | 9060394 | | |
| Kidney Cancer (OD04450- | 1.7 | 2.3 | Gastric Cancer | 6.0 | 2.6 |
| 01) | | | 9060397 | | |
| Kidney Margin | 6.5 | 5.9 | Stomach Margin | 2.1 | 1.5 |
| (OD04450-03) | | | 9060396 | | |
| Kidney Cancer 8120607 | 4.5 | 2.5 | Gastric Cancer 064005 | 10.9 | 10.4 |

TABLE 30
Panel 4.1D

| | Rel. Exp. (%) | | Rel. Exp. (%) |
|---|---|---|---|
| | Ag1544, Run | | Ag1544, Run |
| Tissue Name | 209988812 | Tissue Name | 209988812 |
| Secondary Th1 act | 7.9 | HUVEC IL-1beta | 4.0 |
| Secondary Th2 act | 11.7 | HUVEC IFN gamma | 18.4 |
| Secondary Tr1 act | 7.3 | HUVEC TNF alpha + IFN gamma | 19.5 |
| Secondary Th1 rest | 0.8 | HUVEC TNF alpha + IL4 | 2.8 |
| Secondary Th2 rest | 1.2 | HUVEC IL-11 | 2.0 |
| Secondary Tr1 rest | 1.1 | Lung Microvascular EC none | 7.5 |
| Primary Th1 act | 6.7 | Lung Microvascular EC | 3.5 |
| | | TNFalpha + IL-1beta | |
| Primary Th2 act | 4.5 | Microvascular Dermal EC none | 5.2 |
| Primary Tr1 act | 5.9 | Microsvasular Dermal EC | 1.4 |
| | | TNFalpha + IL-1beta | |
| Primary Th1 rest | 0.4 | Bronchial epithelium TNFalpha + | 1.1 |
| | | IL1beta | |
| Primary Th2 rest | 0.3 | Small airway epithelium none | 0.7 |
| Primary Tr1 rest | 0.3 | Small airway epithelium | 2.3 |
| | | TNFalpha + IL-1beta | |
| CD45RA CD4 lymphocyte act | 5.8 | Coronery artery SMC rest | 1.4 |
| CD45RO CD4 lymphocyte act | 6.5 | Coronery artery SMC TNFalpha + | 1.8 |
| | | IL-1beta | |
| CD8 lymphocyte act | 2.5 | Astrocytes rest | 3.3 |
| Secondary CD8 lymphocyte rest | 5.9 | Astrocytes TNFalpha + IL-1beta | 5.8 |
| Secondary CD8 lymphocyte act | 0.7 | KU-812 (Basophil) rest | 0.0 |
| CD4 lymphocyte none | 0.4 | KU-812 (Basophil) | 0.3 |
| | | PMA/ionomycin | |
| 2ry Th1/Th2/Tr1_anti-CD95 | 1.7 | CCD1106 (Keratinocytes) none | 5.4 |
| CH11 | | | |
| LAK cells rest | 23.8 | CCD1106 (Keratinocytes) | 19.9 |
| | | TNFalpha + IL-1beta | |
| LAK cells IL-2 | 2.0 | Liver cirrhosis | 0.2 |
| LAK cells IL-2 + IL-12 | 3.1 | NCI-H292 none | 0.8 |
| LAK cells IL-2 + IFN gamma | 5.6 | NCI-H292 IL-4 | 1.4 |
| LAK cells IL-2 + IL-18 | 6.6 | NCI-H292 IL-9 | 1.0 |
| LAK cells PMA/ionomycin | 77.9 | NCI-H292 IL-13 | 1.6 |
| NK Cells IL-2 rest | 1.5 | NCI-H292 IFN gamma | 4.2 |
| Two Way MLR 3 day | 21.6 | HPAEC none | 2.4 |
| Two Way MLR 5 day | 18.9 | HPAEC TNF alpha + IL-1 beta | 4.7 |
| Two Way MLR 7 day | 5.1 | Lung fibroblast none | 0.8 |
| PBMC rest | 0.3 | Lung fibroblast TNF alpha + | 1.6 |
| | | IL-1beta | |
| PBMC PWM | 11.5 | Lung fibroblast IL-4 | 1.5 |
| PBMC PHA-L | 8.4 | Lung fibroblast IL-9 | 1.7 |
| Ramos (B cell) none | 0.2 | Lung fibroblast IL-13 | 1.1 |
| Ramos (B cell) ionomycin | 0.3 | Lung fibroblast IFN gamma | 15.0 |
| B lymphocytes PWM | 2.8 | Dermal fibroblast CCD1070 rest | 2.8 |
| B lymphocytes CD40L and IL-4 | 1.2 | Dermal fibroblast CCD1070 TNF | 4.9 |
| | | alpha | |
| EOL-1 dbcAMP | 0.1 | Dermal fibroblast CCD1070 IL-1 | 4.0 |
| | | beta | |
| EOL-1 dbcAMP PMA/ionomycin | 0.2 | Dermal fibroblast IFN gamma | 3.7 |
| Dendritic cells none | 19.9 | Dermal fibroblast IL-4 | 0.7 |
| Dendritic cells LPS | 58.2 | Dermal Fibroblasts rest | 0.2 |
| Dendritic cells anti-CD40 | 12.6 | Neutrophils TNFa + LPS | 0.4 |
| Monocytes rest | 0.1 | Neutrophils rest | 0.6 |
| Monocytes LPS | 100.0 | Colon | 0.2 |
| Macrophages rest | 4.6 | Lung | 0.6 |
| Macrophages LPS | 32.3 | Thymus | 4.5 |
| HUVEC none | 2.0 | Kidney | 0.5 |
| HUVEC starved | 3.2 | | |

Panel 1.2 Summary: Ag1544 The NOV4a gene is most highly expressed in heart (CT=23.4) This gene also has moderate to high levels of expression in several other endocrine/metabolic related tissues, including adrenal, kidney, liver, skeletal muscle, and small intestine. Therefore, a therapeutic modulator to this gene and/or gene product may be useful in the treatment of diseases of endocrine/metabolic origin.

The expression of the NOV4a gene confirms expression in the hippocampus, thalamus, and cerebral cortex. Please see panel 1.3D for a discussion of utility of this gene in the central nervous system.

In addition, there is substantial expression associated with three lung cancer cell lines. Thus, the expression of the NOV4a gene could be used to distinguish heart tissue from the other samples in the panel. Moreover, therapeutic modulation of this gene, through the use of small molecule drugs, antibodies or protein therapeutics might be beneficial in the treatment of lung cancer.

Panel 1.3D Summary: Ag1544 The expression of the NOV4a gene appears to be highest in sample derived from a gastric cancer cell line (NCI-H87) (CT=27.3). In addition, there is substantial expression forund in lung cancer cell lines, a breast cancer cell line and placental tissue. Thus, the expression of this gene could be used to distinguish NCI-H87 cells from other samples in the panel. Moreover, therapeutic modulation of this gene, through the use of small molecule drugs, antibodies or protein therapeutics might be beneficial in the treatment of lung cancer or breast cancer.

Panel 2D Summary: Ag1544 The expression of the NOV4a gene was assessed by two independent runs in panel 2D with very good concordance between the runs. In both runs, there is high expression associated with bladder cancer tissue and lung tissue derived samples. Thus, the expression of the NOV4a gene could be used to distinguish between these samples and the rest of the samples in the panel. Moreover, therapeutic modulation of this gene, through the use of small molecule drugs, antibodies or protein therapeutics might be beneficial in the treatment of bladder or lung cancer.

Panel 4.1D Summary: Ag1544 The NOV4a transcript is is expressed in LAK cells, and induced in LAK cells activated with PMA/ionomycin, dendritic cells treated with LPS, monocytes treated with LPS, Gamma interferon treated HUVEC cells and keratinocytes treated with TNFalpha and IL-1beta. This transcript encodes a smaller isoform of B7-H1, an antigen presentation co-receptor. B7-H 1 binds to PD-1 ligand on T cells, resulting in T cell activation and production of IL-10. Antibody or other types of therapeutics designed with B7-H1 could block T cell activation and be particularly important in the treatment of T cell-mediated diseases such as asthma, psoriasis, IBD and arthritis. Alternatively, agonistic therapeutics could be designed with the NOV4a protein and have adjuvant or immuno-modulatory properties.

References:

Dong H, Zhu G, Tamada K, Chen L. B7-H1, a third member of the B7 family, co-stimulates T-cell proliferation and interleukin-10 secretion. Nat Med 1999 December;5(12):1365-9

The B7 family members B7-1 and B7-2 interact with CD28 and constitute an essential T-cell co-stimulatory pathway in the initiation of antigen-specific humoral and cell-mediated immune response. Here, we describe a third member of the B7 family, called B7-H1 that does not bind CD28, cytotoxic T-lymphocyte A4 or ICOS (inducible co-stimulator). Ligation of B7-H 1 co-stimulated T-cell responses to polyclonal stimuli and allogeneic antigens, and preferentially stimulated the production of interleukin-10. Interleukin-2, although produced in small amounts, was required for the effect of B7-H1 co-stimulation. Our studies thus define a previously unknown co-stimulatory molecule that may be involved in the negative regulation of cell-mediated immune responses.

PMID: 10581077

C. NOV4b: Splice variant of NOV4a, B7H1

Expression of the NOV4b gene (CG56110-04) was assessed using the primer-probe set Ag5282, described in Table 31. Results of the RTQ-PCR runs are shown in Tables 32, 33 and 34.

TABLE 31
Probe Name Ag5282

| | | | | SEQ ID |
|---|---|---|---|---|
| Primers | Sequences | Length | Start Position | NO |
| Forward | 5′-tcaagtcctgagtggagattagat-3′ | 24 | 516 | 186 |
| Probe | TET-5′-tggtcatcccagaactacctctggca-3′-TAMRA | 26 | 565 | 187 |
| Reverse | 5′-cccagaattaccaagtgagtcct-3′ | 23 | 606 | 188 |

TABLE 32
CNS_neurodegeneration_v1.0

| | Rel. Exp. (%) | | Rel. Exp. (%) |
|---|---|---|---|
| | Ag5282, Run | | Ag5282, Run |
| Tissue Name | 233610764 | Tissue Name | 233610764 |
| AD 1 Hippo | 0.0 | Control (Path) 3 Temporal Ctx | 0.0 |
| AD 2 Hippo | 0.0 | Control (Path) 4 Temporal Ctx | 49.0 |
| AD 3 Hippo | 0.0 | AD 1 Occipital Ctx | 0.0 |
| AD 4 Hippo | 0.0 | AD 2 Occipital Ctx (Missing) | 0.0 |
| AD 5 hippo | 0.0 | AD 3 Occipital Ctx | 0.0 |
| AD 6 Hippo | 27.2 | AD 4 Occipital Ctx | 0.0 |
| Control 2 Hippo | 0.0 | AD 5 Occipital Ctx | 72.2 |
| Control 4 Hippo | 100.0 | AD 6 Occipital Ctx | 0.0 |
| Control (Path) 3 Hippo | 0.0 | Control 1 Occipital Ctx | 0.0 |
| AD 1 Temporal Ctx | 0.0 | Control 2 Occipital Ctx | 0.0 |
| AD 2 Temporal Ctx | 0.0 | Control 3 Occipital Ctx | 0.0 |
| AD 3 Temporal Ctx | 0.0 | Control 4 Occipital Ctx | 0.0 |
| AD 4 Temporal Ctx | 0.0 | Control (Path) 1 Occipital Ctx | 42.0 |
| AD 5 Inf Temporal Ctx | 50.7 | Control (Path) 2 Occipital Ctx | 0.0 |
| AD 5 Sup Temporal Ctx | 39.0 | Control (Path) 3 Occipital Ctx | 0.0 |
| AD 6 Inf Temporal Ctx | 52.1 | Control (Path) 4 Occipital Ctx | 0.0 |
| AD 6 Sup Temporal Ctx | 0.0 | Control 1 Parietal Ctx | 0.0 |
| Control 1 Temporal Ctx | 0.0 | Control 2 Parietal Ctx | 53.6 |
| Control 2 Temporal Ctx | 0.0 | Control 3 Parietal Ctx | 0.0 |
| Control 3 Temporal Ctx | 0.0 | Control (Path) 1 Parietal Ctx | 39.0 |
| Control 4 Temporal Ctx | 0.0 | Control (Path) 2 Parietal Ctx | 0.0 |
| Control (Path) 1 Temporal Ctx | 0.0 | Control (Path) 3 Parietal Ctx | 0.0 |
| Control (Path) 2 Temporal Ctx | 0.0 | Control (Path) 4 Parietal Ctx | 0.0 |

TABLE 33
General_screening_panel_v1.5

| | Rel. Exp. (%) | | Rel. Exp. (%) |
|---|---|---|---|
| | Ag5282, Run | | Ag5282, Run |
| Tissue Name | 230565189 | Tissue Name | 230565189 |
| Adipose | 0.0 | Renal ca. TK-10 | 0.7 |
| Melanoma* Hs688(A).T | 0.0 | Bladder | 4.0 |
| Melanoma* Hs688(B).T | 5.1 | Gastric ca. (liver met.) NCI- | 100.0 |
| | | N87 | |
| Melanoma* M14 | 0.6 | Gastric ca. KATO III | 4.2 |
| Melanoma* LOXIMVI | 23.0 | Colon ca. SW-948 | 0.0 |
| Melanoma* SK-MEL-5 | 3.6 | Colon ca. SW480 | 2.2 |
| Squamous cell carcinoma SCC-4 | 10.9 | Colon ca.* (SW480 met) | 0.0 |
| | | SW620 | |
| Testis Pool | 0.0 | Colon ca. HT29 | 0.0 |
| Prostate ca.* (bone met) PC-3 | 0.0 | Colon ca. HCT-116 | 3.6 |
| Prostate Pool | 1.2 | Colon ca. CaCo-2 | 0.0 |
| Placenta | 2.8 | Colon cancer tissue | 1.8 |
| Uterus Pool | 2.8 | Colon ca. SW1116 | 0.0 |
| Ovarian ca. OVCAR-3 | 2.7 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 4.8 |
| Ovarian ca. OVCAR-5 | 6.1 | Small Intestine Pool | 4.3 |
| Ovarian ca. IGROV-1 | 5.4 | Stomach Pool | 0.0 |
| Ovarian ca. OVCAR-8 | 1.3 | Bone Marrow Pool | 0.9 |
| Ovary | 0.0 | Fetal Heart | 3.9 |
| Breast ca. MCF-7 | 0.0 | Heart Pool | 2.3 |
| Breast ca. MDA-MB-231 | 31.2 | Lymph Node Pool | 0.0 |
| Breast ca. BT 549 | 3.6 | Fetal Skeletal Muscle | 1.2 |
| Breast ca. T47D | 0.0 | Skeletal Muscle Pool | 3.1 |
| Breast ca. MDA-N | 3.7 | Spleen Pool | 3.9 |
| Breast Pool | 0.5 | Thymus Pool | 3.9 |
| Trachea | 1.1 | CNS cancer (glio/astro) U87- | 21.9 |
| | | MG | |
| Lung | 1.2 | CNS cancer (glio/astro) U-118- | 3.5 |
| | | MG | |
| Fetal Lung | 1.3 | CNS cancer (neuro; met) SK-N- | 1.4 |
| | | AS | |
| Lung ca. NCI-N417 | 0.0 | CNS cancer (astro) SF-539 | 0.0 |
| Lung ca. LX-1 | 2.7 | CNS cancer (astro) SNB-75 | 2.5 |
| Lung ca. NCI-H146 | 0.0 | CNS cancer (glio) SNB-19 | 8.7 |
| Lung ca. SHP-77 | 0.0 | CNS cancer (glio) SF-295 | 3.6 |
| Lung ca. A549 | 0.0 | Brain (Amygdala) Pool | 0.0 |
| Lung ca. NCI-H526 | 0.0 | Brain (cerebellum) | 1.3 |
| Lung ca. NCI-H23 | 0.8 | Brain (fetal) | 0.0 |
| Lung ca. NCI-H460 | 1.4 | Brain (Hippocampus) Pool | 0.0 |
| Lung ca. HOP-62 | 13.1 | Cerebral Cortex Pool | 2.1 |
| Lung ca. NCI-H522 | 0.0 | Brain (Substantia nigra) Pool | 0.0 |
| Liver | 0.0 | Brain (Thalamus) Pool | 0.9 |
| Fetal Liver | 0.0 | Brain (whole) | 0.0 |
| Liver ca. HepG2 | 0.0 | Spinal Cord Pool | 1.4 |
| Kidney Pool | 3.1 | Adrenal Gland | 2.2 |
| Fetal Kidney | 0.0 | Pituitary gland Pool | 0.0 |
| Renal ca. 786-0 | 5.1 | Salivary Gland | 0.0 |
| Renal ca. A498 | 2.1 | Thyroid (female) | 2.5 |
| Renal ca. ACHN | 1.3 | Pancreatic ca. CAPAN2 | 12.3 |
| Renal ca. UO-31 | 6.8 | Pancreas Pool | 1.8 |

TABLE 34
Panel 4.1D

| | Rel. | | Rel. |
|---|---|---|---|
| | Exp. (%) | | Exp. (%) |
| | Ag5282, | | Ag5282, |
| | Run | | Run |
| Tissue Name | 230510202 | Tissue Name | 230510202 |
| Secondary Th1 act | 5.6 | HUVEC IL-1beta | 1.7 |
| Secondary Th2 act | 18.6 | HUVEC IFN gamma | 25.5 |
| Secondary Tr1 act | 4.9 | HUVEC TNF alpha + IFN gamma | 13.9 |
| Secondary Th1 rest | 0.0 | HUVEC TNF alpha + IL4 | 2.0 |
| Secondary Th2 rest | 0.3 | HUVEC IL-11 | 0.0 |
| Secondary Tr1 rest | 0.9 | Lung Microvascular EC none | 9.9 |
| Primary Th1 act | 4.6 | Lung Microvascular EC TNF alpha + | 0.0 |
| | | IL-1beta | |
| Primary Th2 act | 4.1 | Microvascular Dermal EC none | 0.8 |
| Primary Tr1 act | 9.7 | Microsvasular Dermal EC TNF alpha + | 0.3 |
| | | IL-1beta | |
| Primary Th1 rest | 0.0 | Bronchial epithelium TNF alpha + | 0.3 |
| | | IL1beta | |
| Primary Th2 rest | 0.8 | Small airway epithelium none | 2.3 |
| Primary Tr1 rest | 0.0 | Small airway epithelium TNF alpha + | 2.2 |
| | | IL-1beta | |
| CD45RA CD4 lymphocyte act | 9.8 | Coronery artery SMC rest | 1.0 |
| CD45RO CD4 lymphocyte act | 13.1 | Coronery artery SMC TNF alpha + IL- | 1.9 |
| | | 1beta | |
| CD8 lymphocyte act | 0.0 | Astrocytes rest | 1.8 |
| Secondary CD8 lymphocyte rest | 6.7 | Astrocytes TNF alpha + IL-1beta | 5.3 |
| Secondary CD8 lymphocyte act | 0.8 | KU-812 (Basophil) rest | 0.0 |
| CD4 lymphocyte none | 0.0 | KU-812 (Basophil) PMA/ionomycin | 1.0 |
| 2ry Th1/Th2/Tr1_anti-CD95 CH11 | 0.0 | CCD1106 (Keratinocytes) none | 5.5 |
| LAK cells rest | 14.0 | CCD1106 (Keratinocytes) TNF alpha + | 18.8 |
| | | IL-1beta | |
| LAK cells IL-2 | 0.0 | Liver cirrhosis | 0.0 |
| LAK cells IL-2 + IL-12 | 1.9 | NCI-H292 none | 0.0 |
| LAK cells IL-2 + IFN gamma | 1.7 | NCI-H292 IL-4 | 1.0 |
| LAK cells IL-2 + IL-18 | 3.0 | NCI-H292 IL-9 | 1.0 |
| LAK cells PMA/ionomycin | 76.3 | NCI-H292 IL-13 | 1.5 |
| NK Cells IL-2 rest | 1.0 | NCI-H292 IFN gamma | 8.8 |
| Two Way MLR 3 day | 9.4 | HPAEC none | 0.9 |
| Two Way MLR 5 day | 4.0 | HPAEC TNF alpha + IL-1beta | 6.0 |
| Two Way MLR 7 day | 0.7 | Lung fibroblast none | 0.0 |
| PBMC rest | 0.0 | Lung fibroblast TNF alpha + IL-1beta | 1.0 |
| PBMC PWM | 6.1 | Lung fibroblast IL-4 | 2.3 |
| PBMC PHA-L | 6.1 | Lung fibroblast IL-9 | 1.6 |
| Ramos (B cell) none | 0.0 | Lung fibroblast IL-13 | 0.0 |
| Ramos (B cell) ionomycin | 0.0 | Lung fibroblast IFN gamma | 17.0 |
| B lymphocytes PWM | 3.6 | Dermal fibroblast CCD1070 rest | 6.6 |
| B lymphocytes CD40L and IL-4 | 0.0 | Dermal fibroblast CCD1070 TNF | 9.4 |
| | | alpha | |
| EOL-1 dbcAMP | 0.0 | Dermal fibroblast CCD1070 IL-1beta | 5.3 |
| EOL-1 dbcAMP PMA/ionomycin | 0.0 | Dermal fibroblast IFN gamma | 2.8 |
| Dendritic cells none | 10.2 | Dermal fibroblast IL-4 | 0.0 |
| Dendritic cells LPS | 36.1 | Dermal Fibroblasts rest | 0.0 |
| Dendritic cells anti-CD40 | 4.8 | Neutrophils TNFa + LPS | 0.9 |
| Monocytes rest | 0.0 | Neutrophils rest | 0.0 |
| Monocytes LPS | 100.0 | Colon | 0.0 |
| Macrophages rest | 2.6 | Lung | 0.0 |
| Macrophages LPS | 5.0 | Thymus | 0.0 |
| HUVEC none | 2.2 | Kidney | 0.0 |
| HUVEC starved | 3.8 | | |

CNS_neurodegeneration_v1.0 Summary: Ag5282 Expression of the NOV4b gene is low/undetectable in all samples on this panel (CTs>35). (Data not shown.)

General_screening_panel_v1.5 Summary: Ag5282 The expression of the NOV4b gene appears to be highest in a sample derived from a gastric cancer cell line (NCI-H87)(CT=3 1). Overall, there is relatively low expression in the remaining samples of panel 1.5. Thus, the expression of this gene could be used to distinguish NCI-H87 cells from other samples in the panel.

Panel 4.1D Summary: Ag5282 The NOV4b transcript is not expressed in the normal tissue samples on this panel. The transcript is expressed in LAK cells, and induced in LAK cells activated with PMA/ionomycin, dendritic cells treated with LPS, monocytes treated with LPS, Gamma interferon treated HUVEC cells and keratinocytes treated with TNFalpha and IL-1beta. The NOV4b transcript encodes a smaller isoform of B7-H1, an antigen presentation co-receptor. B7-H1 binds to PD-1 ligand on T cells and resulting in T cell activation and production of IL-10. Antibody or other types of therapeutics designed with B7-H1 could block T cell activation and be particularly important in the treatment of T cell-mediated diseases such as asthma, psoriasis, IBD and arthritis. Alternatively, agonistic therapeutics could be designed with this protein and have adjuvant like properties.

References:

Dong H, Zhu G, Tamada K, Chen L. B7-H1, a third member of the B7 family, co-stimulates T-cell proliferation and interleukin-10 secretion. Nat Med 1999 December;5(12):1365-9

The B7 family members B7-1 and B7-2 interact with CD28 and constitute an essential T-cell co-stimulatory pathway in the initiation of antigen-specific humoral and cell-mediated immune response. Here, we describe a third member of the B7 family, called B7-H1 that does not bind CD28, cytotoxic T-lymphocyte A4 or ICOS (inducible co-stimulator). Ligation of B7-H1 co-stimulated T-cell responses to polyclonal stimuli and allogeneic antigens, and preferentially stimulated the production of interleukin-10. Interleukin-2, although produced in small amounts, was required for the effect of B7-H1 co-stimulation. Our studies thus define a previously unknown co-stimulatory molecule that may be involved in the negative regulation of cell-mediated immune responses.

PMID: 10581077

D. NOV5a: Prostasin

Expression of the NOV5a gene (CG56142-01) was assessed using the primer-probe set Ag2888, described in Table 35. Results of the RTQ-PCR runs are shown in Tables 36, 37 and 38.

TABLE 35
Probe Name Ag2888

| | | | | SEQ ID |
|---|---|---|---|---|
| Primers | Sequences | Length | Start Position | NO: |
| Forward | 5′-aatgagaggggtttcctgtct-3′ | 21 | 18 | 189 |
| Probe | TET-5′-caggtcctgctccttctggtgctg-3′-TAMRA | 24 | 40 | 190 |
| Reverse | 5′-caacgatccgactggacat-3′ | 19 | 82 | 191 |

TABLE 36
Panel 1.3D

| | Rel. | Rel. | | Rel. | Rel. |
|---|---|---|---|---|---|
| | Exp. (%) | Exp. (%) | | Exp. (%) | Exp. (%) |
| | Ag2888, | Ag2888, | | Ag2888, | Ag2888, |
| | Run | Run | | Run | Run |
| Tissue Name | 160857626 | 165721688 | Tissue Name | 160857626 | 165721688 |
| Liver adenocarcinoma | 0.0 | 0.0 | Kidney (fetal) | 0.0 | 0.0 |
| Pancreas | 0.0 | 0.0 | Renal ca. 786-0 | 0.0 | 0.0 |
| Pancreatic ca. CAPAN2 | 0.0 | 0.0 | Renal ca. A498 | 0.0 | 0.0 |
| Adrenal gland | 0.0 | 0.0 | Renal ca. RXF 393 | 0.0 | 0.0 |
| Thyroid | 0.0 | 0.0 | Renal ca. ACHN | 0.0 | 0.0 |
| Salivary gland | 0.0 | 0.0 | Renal ca. UO-31 | 0.0 | 0.0 |
| Pituitary gland | 0.0 | 0.0 | Renal ca. TK-10 | 0.0 | 0.0 |
| Brain (fetal) | 0.0 | 0.0 | Liver | 0.0 | 0.0 |
| Brain (whole) | 0.0 | 0.0 | Liver (fetal) | 0.0 | 0.0 |
| Brain (amygdala) | 0.0 | 0.0 | Liver ca. (hepatoblast) | 0.0 | 0.0 |
| | | | HepG2 | | |
| Brain (cerebellum) | 0.0 | 0.0 | Lung | 0.0 | 0.0 |
| Brain (hippocampus) | 0.0 | 0.0 | Lung (fetal) | 0.0 | 0.0 |
| Brain (substantia nigra) | 0.0 | 0.0 | Lung ca. (small cell) | 0.0 | 0.0 |
| | | | LX-1 | | |
| Brain (thalamus) | 0.0 | 0.0 | Lung ca. (small cell) | 0.0 | 0.0 |
| | | | NCI-H69 | | |
| Cerebral Cortex | 0.0 | 0.0 | Lung ca. (s. cell var.) | 0.0 | 0.0 |
| | | | SHP-77 | | |
| Spinal cord | 0.0 | 0.0 | Lung ca. (large | 0.0 | 0.0 |
| | | | cell)NCI-H460 | | |
| glio/astro U87-MG | 0.0 | 0.0 | Lung ca. (non-sm. cell) | 0.0 | 0.0 |
| | | | A549 | | |
| glio/astro U-118-MG | 0.0 | 0.6 | Lung ca. (non-s. cell) | 0.0 | 0.0 |
| | | | NCI-H23 | | |
| astrocytoma SW1783 | 0.0 | 0.0 | Lung ca. (non-s. cell) | 0.0 | 0.0 |
| | | | HOP-62 | | |
| neuro*; met SK-N-AS | 0.0 | 0.0 | Lung ca. (non-s. cl) | 0.0 | 0.0 |
| | | | NCI-H522 | | |
| astrocytoma SF-539 | 0.0 | 0.0 | Lung ca. (squam.) SW | 0.0 | 0.0 |
| | | | 900 | | |
| astrocytoma SNB-75 | 0.0 | 0.0 | Lung ca. (squam.) | 0.0 | 0.0 |
| | | | NCI-H596 | | |
| glioma SNB-19 | 10.3 | 4.8 | Mammary gland | 0.0 | 0.0 |
| glioma U251 | 1.4 | 0.6 | Breast ca.* (pl. ef) | 0.0 | 0.0 |
| | | | MCF-7 | | |
| glioma SF-295 | 0.0 | 0.0 | Breast ca.* (pl. ef) | 0.0 | 0.0 |
| | | | MDA-MB-231 | | |
| Heart (fetal) | 0.0 | 0.0 | Breast ca.* (pl. ef) | 0.0 | 0.0 |
| | | | T47D | | |
| Heart | 0.0 | 0.0 | Breast ca. BT-549 | 0.0 | 0.0 |
| Skeletal muscle (fetal) | 0.0 | 0.0 | Breast ca. MDA-N | 0.0 | 0.6 |
| Skeletal muscle | 0.0 | 0.0 | Ovary | 1.6 | 0.0 |
| Bone marrow | 0.8 | 0.0 | Ovarian ca. OVCAR-3 | 0.0 | 0.0 |
| Thymus | 0.0 | 0.0 | Ovarian ca. OVCAR-4 | 0.0 | 0.0 |
| Spleen | 0.0 | 0.0 | Ovarian ca. OVCAR-5 | 0.0 | 0.0 |
| Lymph node | 0.0 | 0.0 | Ovarian ca. OVCAR-8 | 0.0 | 0.0 |
| Colorectal | 0.0 | 0.0 | Ovarian ca. IGROV-1 | 0.0 | 0.0 |
| Stomach | 3.4 | 0.0 | Ovarian ca.* (ascites) | 0.0 | 0.4 |
| | | | SK-OV-3 | | |
| Small intestine | 0.8 | 0.0 | Uterus | 0.0 | 0.0 |
| Colon ca. SW480 | 1.7 | 0.4 | Placenta | 0.0 | 0.0 |
| Colon ca.* SW620(SW480 | 0.0 | 0.0 | Prostate | 0.0 | 0.0 |
| met) | | | | | |
| Colon ca. HT29 | 0.0 | 0.0 | Prostate ca.* (bone | 0.0 | 0.0 |
| | | | met)PC-3 | | |
| Colon ca. HCT-116 | 0.0 | 0.0 | Testis | 1.4 | 0.0 |
| Colon ca. CaCo-2 | 0.0 | 0.0 | Melanoma Hs688(A).T | 0.0 | 0.0 |
| Colon ca. tissue(ODO3866) | 100.0 | 100.0 | Melanoma* (met) | 0.0 | 0.0 |
| | | | Hs688(B).T | | |
| Colon ca. HCC-2998 | 0.0 | 0.0 | Melanoma UACC-62 | 0.0 | 0.9 |
| Gastric ca.* (liver met) NCI- | 0.0 | 0.0 | Melanoma M14 | 0.0 | 0.0 |
| N87 | | | | | |
| Bladder | 0.0 | 0.0 | Melanoma LOX IMVI | 0.0 | 0.0 |
| Trachea | 0.0 | 0.0 | Melanoma* (met) SK- | 0.0 | 0.0 |
| | | | MEL-5 | | |
| Kidney | 0.0 | 0.0 | Adipose | 0.0 | 0.0 |

TABLE 37
Panel 2D

| | Rel. | | Rel. |
|---|---|---|---|
| | Exp. (%) | | Exp. (%) |
| | Ag2888, | | Ag2888, |
| | Run | | Run |
| Tissue Name | 160897960 | Tissue Name | 160897960 |
| Normal Colon | 0.0 | Kidney Margin 8120608 | 0.0 |
| CC Well to Mod Diff (ODO3866) | 100.0 | Kidney Cancer 8120613 | 0.0 |
| CC Margin (ODO3866) | 1.1 | Kidney Margin 8120614 | 0.0 |
| CC Gr.2 rectosigmoid (ODO3868) | 0.0 | Kidney Cancer 9010320 | 0.5 |
| CC Margin (ODO3868) | 0.0 | Kidney Margin 9010321 | 0.0 |
| CC Mod Diff (ODO3920) | 3.5 | Normal Uterus | 0.0 |
| CC Margin (ODO3920) | 0.0 | Uterus Cancer 064011 | 0.0 |
| CC Gr.2 ascend colon (ODO3921) | 1.2 | Normal Thyroid | 0.0 |
| CC Margin (ODO3921) | 0.1 | Thyroid Cancer 064010 | 0.0 |
| CC from Partial Hepatectomy | 14.2 | Thyroid Cancer A302152 | 0.0 |
| (ODO4309) Mets | | | |
| Liver Margin (ODO4309) | 0.2 | Thyroid Margin A302153 | 0.0 |
| Colon mets to lung (OD04451-01) | 3.5 | Normal Breast | 0.0 |
| Lung Margin (OD04451-02) | 0.0 | Breast Cancer (OD04566) | 0.0 |
| Normal Prostate 6546-1 | 0.0 | Breast Cancer (OD04590-01) | 0.0 |
| Prostate Cancer (OD04410) | 0.0 | Breast Cancer Mets (OD04590-03) | 0.0 |
| Prostate Margin (OD04410) | 0.0 | Breast Cancer Metastasis | 0.0 |
| | | (OD04655-05) | |
| Prostate Cancer (OD04720-01) | 0.0 | Breast Cancer 064006 | 0.0 |
| Prostate Margin (OD04720-02) | 0.0 | Breast Cancer 1024 | 0.0 |
| Normal Lung 061010 | 0.0 | Breast Cancer 9100266 | 0.0 |
| Lung Met to Muscle (ODO4286) | 0.0 | Breast Margin 9100265 | 0.0 |
| Muscle Margin (ODO4286) | 0.0 | Breast Cancer A209073 | 0.0 |
| Lung Malignant Cancer (OD03126) | 0.2 | Breast Margin A2090734 | 0.0 |
| Lung Margin (OD03126) | 0.0 | Normal Liver | 0.0 |
| Lung Cancer (OD04404) | 0.0 | Liver Cancer 064003 | 0.0 |
| Lung Margin (OD04404) | 0.0 | Liver Cancer 1025 | 0.2 |
| Lung Cancer (OD04565) | 0.0 | Liver Cancer 1026 | 0.0 |
| Lung Margin (OD04565) | 0.0 | Liver Cancer 6004-T | 0.0 |
| Lung Cancer (OD04237-01) | 0.2 | Liver Tissue 6004-N | 0.0 |
| Lung Margin (OD04327-02) | 0.0 | Liver Cancer 6005-T | 0.0 |
| Ocular Mel Met to Liver (ODO4310) | 0.0 | Liver Tissue 6005-N | 0.0 |
| Liver Margin (ODO4310) | 0.0 | Normal Bladder | 0.0 |
| Melanoma Mets to Lung (OD04321) | 0.7 | Bladder Cancer 1023 | 0.0 |
| Lung Margin (OD04321) | 0.0 | Bladder Cancer A302173 | 0.0 |
| Normal Kidney | 0.0 | Bladder Cancer (OD04718-01) | 0.3 |
| Kidney Ca, Nuclear grade 2 (OD04338) | 0.0 | Bladder Normal Adjacent | 0.0 |
| | | (OD04718-03) | |
| Kidney Margin (OD04338) | 0.0 | Normal Ovary | 0.4 |
| Kidney Ca Nuclear grade 1/2 (OD04339) | 0.0 | Ovarian Cancer 064008 | 0.0 |
| Kidney Margin (OD04339) | 0.0 | Ovarian Cancer (OD04768-07) | 0.1 |
| Kidney Ca, Clear cell type (OD04340) | 0.0 | Ovarian Margin (OD04768-08) | 0.0 |
| Kidney Margin (OD04340) | 0.0 | Normal Stomach | 0.7 |
| Kidney Ca, Nuclear grade 3 (OD04348) | 0.3 | Gastric Cancer 9060358 | 0.0 |
| Kidney Margin (OD04348) | 0.0 | Stomach Margin 9060359 | 0.5 |
| Kidney Cancer (OD04622-01) | 0.0 | Gastric Cancer 9060395 | 0.0 |
| Kidney Margin (OD04622-03) | 0.0 | Stomach Margin 9060394 | 0.0 |
| Kidney Cancer (OD04450-01) | 0.0 | Gastric Cancer 9060397 | 17.6 |
| Kidney Margin (OD04450-03) | 0.0 | Stomach Margin 9060396 | 0.0 |
| Kidney Cancer 8120607 | 0.0 | Gastric Cancer 064005 | 0.0 |

TABLE 38
Panel 3D

| | Rel. | | Rel. |
|---|---|---|---|
| | Exp. (%) | | Exp. (%) |
| | Ag2888, | | Ag2888, |
| | Run | | Run |
| Tissue Name | 164629839 | Tissue Name | 164629839 |
| Daoy-Medulloblastoma | 0.0 | Ca Ski-Cervical epidermoid carcinoma | 0.0 |
| | | (metastasis) | |
| TE671-Medulloblastoma | 0.0 | ES-2-Ovarian clear cell carcinoma | 0.0 |
| D283 Med-Medulloblastoma | 0.0 | Ramos-Stimulated with PMA/ionomycin | 0.0 |
| | | 6 h | |
| PFSK-1-Primitive Neuroectodermal | 0.0 | Ramos-Stimulated with PMA/ionomycin | 0.0 |
| | | 14h | |
| XF-498-CNS | 0.0 | MEG-01-Chronic myelogenous | 0.0 |
| | | leukemia (megokaryoblast) | |
| SNB-78-Glioma | 0.0 | Raji-Burkitt's lymphoma | 0.0 |
| SF-268-Glioblastoma | 0.0 | Daudi-Burkitt's lymphoma | 0.0 |
| T98G-Glioblastoma | 0.0 | U266-B-cell plasmacytoma | 0.0 |
| SK-N-SH-Neuroblastoma | 0.0 | CA46-Burkitt's lymphoma | 0.0 |
| (metastasis) | | | |
| SF-295-Glioblastoma | 0.0 | RL-non-Hodgkin's B-cell lymphoma | 0.0 |
| Cerebellum | 0.0 | JM1-pre-B-cell lymphoma | 0.0 |
| Cerebellum | 0.0 | Jurkat-T cell leukemia | 0.0 |
| NCI-H292-Mucoepidermoid lung | 0.0 | TF-1-Erythroleukemia | 0.0 |
| carcinoma | | | |
| DMS-114-Small cell lung cancer | 0.0 | HUT 78-T-cell lymphoma | 0.0 |
| DMS-79-Small cell lung cancer | 0.0 | U937-Histiocytic lymphoma | 0.0 |
| NCI-H146-Small cell lung cancer | 0.0 | KU-812-Myelogenous leukemia | 0.0 |
| NCI-H526-Small cell lung cancer | 0.0 | 769-P-Clear cell renal carcinoma | 0.0 |
| NCI-N417-Small cell lung cancer | 0.0 | Caki-2-Clear cell renal carcinoma | 0.0 |
| NCI-H82-Small cell lung cancer | 0.0 | SW 839-Clear cell renal carcinoma | 0.0 |
| NCI-H157-Squamous cell lung | 0.0 | G401-Wilms' tumor | 0.0 |
| cancer (metastasis) | | | |
| NCI-H1155-Large cell lung cancer | 0.0 | Hs766T-Pancreatic carcinoma (LN | 77.9 |
| | | metastasis) | |
| NCI-H1299-Large cell lung cancer | 0.0 | CAPAN-1-Pancreatic adenocarcinoma | 1.6 |
| | | (liver metastasis) | |
| NCI-H727-Lung carcinoid | 0.0 | SU86.86-Pancreatic carcinoma (liver | 2.5 |
| | | metastasis) | |
| NCI-UMC-11-Lung carcinoid | 0.0 | BxPC-3-Pancreatic adenocarcinoma | 0.0 |
| LX-1-Small cell lung cancer | 0.0 | HPAC-Pancreatic adenocarcinoma | 0.0 |
| Colo-205-Colon cancer | 0.0 | MIA PaCa-2-Pancreatic carcinoma | 0.0 |
| KM12-Colon cancer | 0.0 | CFPAC-1-Pancreatic ductal | 0.0 |
| | | adenocarcinoma | |
| KM20L2-Colon cancer | 0.0 | PANC-1-Pancreatic epithelioid ductal | 0.0 |
| | | carcinoma | |
| NCI-H716-Colon cancer | 0.0 | T24-Bladder carcinma (transitional cell) | 0.0 |
| SW-48-Colon adenocarcinoma | 0.0 | 5637-Bladder carcinoma | 0.0 |
| SW1116-Colon adenocarcinoma | 0.0 | HT-1197-Bladder carcinoma | 0.0 |
| LS 174T-Colon adenocarcinoma | 0.0 | UM-UC-3-Bladder carcinma | 0.0 |
| | | (transitional cell) | |
| SW-948-Colon adenocarcinoma | 0.0 | A204-Rhabdomyosarcoma | 0.0 |
| SW-480-Colon adenocarcinoma | 0.0 | HT-1080-Fibrosarcoma | 0.0 |
| NCI-SNU-5-Gastric carcinoma | 100.0 | MG-63-Osteosarcoma | 0.0 |
| KATO III-Gastric carcinoma | 0.0 | SK-LMS-1-Leiomyosarcoma (vulva) | 0.0 |
| NCI-SNU-16-Gastric carcinoma | 0.0 | SJRH30-Rhabdomyosarcoma (met to | 0.0 |
| | | bone marrow) | |
| NCI-SNU-1-Gastric carcinoma | 0.0 | A431-Epidermoid carcinoma | 0.0 |
| RF-1-Gastric adenocarcinoma | 0.0 | WM266-4-Melanoma | 0.0 |
| RF-48-Gastric adenocarcinoma | 0.0 | DU 145-Prostate carcinoma (brain | 0.0 |
| | | metastasis) | |
| MKN-45-Gastric carcinoma | 0.0 | MDA-MB-468-Breast adenocarcinoma | 0.0 |
| NCI-N87-Gastric carcinoma | 27.9 | SCC-4-Squamous cell carcinoma of | 0.0 |
| | | tongue | |
| OVCAR-5-Ovarian carcinoma | 0.0 | SCC-9-Squamous cell carcinoma of | 0.0 |
| | | tongue | |
| RL95-2-Uterine carcinoma | 0.0 | SCC-15-Squamous cell carcinoma of | 0.0 |
| | | tongue | |
| HelaS3-Cervical adenocarcinoma | 0.0 | CAL 27-Squamous cell carcinoma of | 0.0 |
| | | tongue | |

Panel 1.3D Summary: Ag2888 The expression of the NOV5a gene was assessed in two independent runs in panel 1.3D. The expression of this gene appears to be highest and almost exclusive to a sample derived from a colon cancer (CTs=31). Thus, the expression of the NOV5a gene could be used to distinguish this sample from other samples in the panel. Moreover, therapeutic modulation of this gene, through the use of small molecule drugs, antibodies or protein therapeutics might be beneficial in the treatment of colon cancer.

Panel 2D Summary: Ag2888 The expression of the NOV5a gene appears to be highest and almost exclusive to a sample derived from a colon cancer (CT=30). This expression is consistent with the expression in panel 1.3D. Thus, the expression of the NOV5a gene could be used to distinguish this sample from other samples in the panel. Moreover, therapeutic modulation of this gene, through the use of small molecule drugs, antibodies or protein therapeutics might be beneficial in the treatment of colon cancer.

Panel 3D Summary: Ag2888 The expression of the NOV5a gene appears to be highest and almost exclusive to a sample derived from a gastric cancer cell line (CT=34.1). Thus, the expression of this gene could be used to distinguish this sample from other samples in the panel. Moreover, therapeutic modulation of the NOV5a gene, through the use of small molecule drugs, antibodies or protein therapeutics might be beneficial in the treatment of gastric cancer.

Panel 4D Summary: Ag2888 Expression of the NOV5a gene is low/undetectable in all samples on this panel (CTs>35). (Data not shown.)

E. NOV5b: Prostasin

Expression of the NOV5b gene (CG56142-02) was assessed using the primer-probe set Ag4095, described in Table 39. Results of the RTQ-PCR runs are shown in Tables 40, 41 and 42.

TABLE 39
Probe Name Ag4095

| Primers | Sequences | Length | Start Position | SEQ ID NO: |
|---|---|---|---|---|
| Forward | 5′-aatgagaggggtttcctgtct-3′ | 21 | 90 | 192 |
| Probe | TET-5′-caggtcctgctccttctggtgctg-3′-TAMRA | 24 | 112 | 193 |
| Reverse | 5′-gcagacttccttccctgagt-3′ | 20 | 148 | 194 |

TABLE 40
CNS_neurodegeneration_v1.0

| | Rel. | | Rel. |
|---|---|---|---|
| | Exp. (%) | | Exp. (%) |
| | Ag4095, | | Ag4095, |
| | Run | | Run |
| Tissue Name | 214296164 | Tissue Name | 214296164 |
| AD 1 Hippo | 0.0 | Control (Path) 3 Temporal Ctx | 0.0 |
| AD 2 Hippo | 0.0 | Control (Path) 4 Temporal Ctx | 0.0 |
| AD 3 Hippo | 0.0 | AD 1 Occipital Ctx | 0.0 |
| AD 4 Hippo | 0.0 | AD 2 Occipital Ctx (Missing) | 0.0 |
| AD 5 Hippo | 0.0 | AD 3 Occipital Ctx | 0.0 |
| AD 6 Hippo | 0.0 | AD 4 Occipital Ctx | 0.0 |
| Control 2 Hippo | 0.0 | AD 5 Occipital Ctx | 0.0 |
| Control 4 Hippo | 0.0 | AD 6 Occipital Ctx | 0.0 |
| Control (Path) 3 Hippo | 0.0 | Control 1 Occipital Ctx | 0.0 |
| AD 1 Temporal Ctx | 0.0 | Control 2 Occipital Ctx | 0.0 |
| AD 2 Temporal Ctx | 0.0 | Control 3 Occipital Ctx | 0.0 |
| AD 3 Temporal Ctx | 0.0 | Control 4 Occipital Ctx | 0.0 |
| AD 4 Temporal Ctx | 0.0 | Control (Path) 1 Occipital Ctx | 0.0 |
| AD 5 Inf Temporal Ctx | 0.0 | Control (Path) 2 Occipital Ctx | 0.0 |
| AD 5 Sup Temporal Ctx | 0.0 | Control (Path) 3 Occipital Ctx | 0.0 |
| AD 6 Inf Temporal Ctx | 0.0 | Control (Path) 4 Occipital Ctx | 0.0 |
| AD 6 Sup Temporal Ctx | 0.0 | Control 1 Parietal Ctx | 0.0 |
| Control 1 Temporal Ctx | 100.0 | Control 2 Parietal Ctx | 0.0 |
| Control 2 Temporal Ctx | 0.0 | Control 3 Parietal Ctx | 0.0 |
| Control 3 Temporal Ctx | 0.0 | Control (Path) 1 Parietal Ctx | 0.0 |
| Control 3 Temporal Ctx | 0.0 | Control (Path) 2 Parietal Ctx | 0.0 |
| Control (Path) 1 Temporal Ctx | 0.0 | Control (Path) 3 Parietal Ctx | 0.0 |
| Control (Path) 2 Temporal Ctx | 0.0 | Control (Path) 4 Parietal Ctx | 0.0 |

TABLE 41
General_screening_panel_v1.4

| | Rel. | | Rel. |
|---|---|---|---|
| | Exp. (%) | | Exp. (%) |
| | Ag4095, | | Ag4095, |
| | Run | | Run |
| Tissue Name | 219575329 | Tissue Name | 219575329 |
| Adipose | 0.0 | Renal ca. TK-10 | 0.2 |
| Melanoma* Hs688(A).T | 0.0 | Bladder | 0.0 |
| Melanoma* Hs688(B).T | 0.0 | Gastric ca. (liver met.) NCI-N87 | 0.1 |
| Melanoma* M14 | 0.0 | Gastric ca. KATO III | 1.3 |
| Melanoma* LOXIMVI | 0.0 | Colon ca. SW-948 | 0.2 |
| Melanoma* SK-MEL-5 | 0.0 | Colon ca. SW480 | 2.9 |
| Squamous cell carcinoma SCC-4 | 0.0 | Colon ca.* (SW480 met) SW620 | 0.0 |
| Testis Pool | 0.0 | Colon ca. HT29 | 0.0 |
| Prostate ca.* (bone met) PC-3 | 0.0 | Colon ca. HCT-116 | 0.0 |
| Prostate Pool | 0.0 | Colon ca. CaCo-2 | 0.0 |
| Placenta | 0.0 | Colon cancer tissue | 100.0 |
| Uterus Pool | 0.0 | Colon ca. SW1116 | 0.1 |
| Ovarian ca. OVCAR-3 | 0.0 | Colon ca. Colo-205 | 0.0 |
| Ovarian ca. SK-OV-3 | 0.0 | Colon ca. SW-48 | 0.1 |
| Ovarian ca. OVCAR-4 | 0.0 | Colon Pool | 0.0 |
| Ovarian ca. OVCAR-5 | 0.0 | Small Intestine Pool | 0.0 |
| Ovarian ca. IGROV-1 | 7.5 | Stomach Pool | 0.0 |
| Ovarian ca. OVCAR-8 | 2.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB-231 | 0.2 | Lymph Node Pool | 0.0 |
| Breast ca. BT 549 | 0.0 | Fetal Skeletal Muscle | 0.0 |
| Breast ca. T47D | 0.5 | Skeletal Muscle Pool | 0.0 |
| Breast ca. MDA-N | 0.2 | Spleen Pool | 0.0 |
| Breast Pool | 0.0 | Thymus Pool | 0.0 |
| Trachea | 0.0 | CNS cancer (glio/astro) U87-MG | 0.0 |
| Lung | 1.2 | CNS cancer (glio/astro) U-118-MG | 0.0 |
| Fetal Lung | 0.0 | CNS cancer (neuro; met) SK-N-AS | 0.0 |
| Lung ca. NCI-N417 | 0.0 | CNS cancer (astro) SF-539 | 0.0 |
| Lung ca. LX-1 | 0.0 | CNS cancer (astro) SNB-75 | 0.2 |
| Lung ca. NCI-H146 | 0.0 | CNS cancer (glio) SNB-19 | 6.3 |
| Lung ca. SHP-77 | 0.7 | CNS cancer (glio) SF-295 | 0.5 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) Pool | 0.1 |
| Liver | 0.0 | Brain (Thalamus) Pool | 0.0 |
| Fetal Liver | 0.1 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 0.1 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

TABLE 42
Panel 4.1D

| | Rel. | | Rel. |
|---|---|---|---|
| | Exp. (%) | | Exp. (%) |
| | Ag4095, | | Ag4095, |
| | Run | | Run |
| Tissue Name | 172383943 | Tissue Name | 172383943 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + | 0.0 |
| | | IL-1beta | |
| Primary Th2 act | 0.0 | Microvascular Dermal EC none | 0.0 |
| Primary Tr1 act | 0.0 | Microsvasular Dermal EC TNF alpha + | 0.0 |
| | | IL-1beta | |
| Primary Th1 rest | 0.0 | Bronchial epithelium TNF alpha + | 0.0 |
| | | IL1beta | |
| Primary Th2 rest | 0.0 | Small airway epithelium none | 0.0 |
| Primary Tr1 rest | 0.0 | Small airway epithelium TNF alpha + | 0.0 |
| | | IL-1beta | |
| CD45RA CD4 lymphocyte act | 0.0 | Coronery artery SMC rest | 0.0 |
| CD45RO CD4 lymphocyte act | 0.0 | Coronery artery SMC TNF alpha + | 0.0 |
| | | IL-1beta | |
| CD8 lymphocyte act | 0.0 | Astrocytes rest | 8.3 |
| Secondary CD8 lymphocyte rest | 0.0 | Astrocytes TNF alpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.0 |
| 2ry Th1/Th2/Tr1_anti-CD95 CH11 | 0.0 | CCD1106 (Keratinocytes) none | 0.0 |
| LAK cells rest | 0.0 | CCD1106 (Keratinocytes) TNF alpha + | 0.0 |
| | | IL-1beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN gamma | 0.0 | NCI-H292 IL-4 | 0.0 |
| LAK cells IL-2 + IL-18 | 0.0 | NCI-H292 IL-9 | 0.0 |
| LAK cells PMA/ionomycin | 0.0 | NCI-H292 IL-13 | 0.0 |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1beta | 0.0 |
| Two Way MLR 7 day | 0.0 | Lung fibroblast none | 0.0 |
| PBMC rest | 0.0 | Lung fibroblast TNF alpha + IL- | 0.0 |
| | | 1beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 0.0 | Dermal fibroblast CCD1070 rest | 0.0 |
| B lymphocytes CD40L and IL-4 | 0.0 | Dermal fibroblast CCD1070 TNF | 0.0 |
| | | alpha | |
| EOL-1 dbcAMP | 0.0 | Dermal fibroblast CCD1070 IL- | 0.0 |
| | | 1beta | |
| EOL-1 dbcAMP PMA/ionomycin | 0.0 | Dermal fibroblast IFN gamma | 0.0 |
| Dendritic cells none | 0.0 | Dermal fibroblast IL-4 | 3.9 |
| Dendritic cells LPS | 0.0 | Dermal Fibroblasts rest | 0.0 |
| Dendritic cells anti-CD40 | 0.0 | Neutrophils TNFa + LPS | 17.9 |
| Monocytes rest | 0.0 | Neutrophils rest | 100.0 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

CNS_neurodegeneration_v1.0 Summary: Ag4095 Expression of the NOV5b gene is low/undetectable in all samples on this panel (CTs>35). (Data not shown.)

General_screening_panel_v1.4 Summary: Ag4095 The expression of the NOV5b gene appears to be highest and almost exclusive to a sample derived from a colon cancer (CT=27). Thus, the expression of this gene could be used to distinguish this colon cancer sample from other samples in the panel. Moreover, therapeutic modulation of the NOV5b gene, through the use of small molecule drugs, protein therapeutics or antibodies might be beneficial in the treatment of colon cancer.

Panel 4.1D Summary: Ag4095 The NOV5b gene, a prostasin homolog, is expressed almost exclusively in resting neutrophils. This expression is reduced nearly to the background level (CT=34.18) in neutrophils activated by TNF-alpha+LPS. This expression profile suggests that the serine proteinase homolog encoded by the NOV5b gene is produced by resting neutrophils but not by activated neutrophils. Therefore, the NOV5b gene product may reduce activation of these inflammatory cells and be useful as a protein therapeutic to reduce or eliminate the symptoms in patients with Crohn's disease, ulcerative colitis, multiple sclerosis, chronic obstructive pulmonary disease, asthma, emphysema, rheumatoid arthritis, lupus erythematosus, or psoriasis.

In addition, small molecule or antibody antagonists of the NOV5b gene product may be effective in increasing the immune response in patients with AIDS or other immunodeficiencies.

F. NOV6 (NOV6a,NOV6b and NOV6c): Lysosomal Acid Lipase Precursor

Expression of the NOV6a and NOV6b genes (CG50159-01 and CG50159-02) was assessed using the primer-probe sets Ag1456, Ag2446, Ag2132, Ag2444, Ag1899 and Ag2059, described in Tables 43, 44, 45, 46, 47 and 48. Results of the RTQ-PCR runs are shown in Tables 52, 53, and 54. Please note that the probe and primer sets Ag2059, Ag2132, Ag2444, Ag2446 do not correspond to the NOV6b variant. The probe and primer set Ag2919, described in Table 49, do not correspond to NOV6a. NOV6c (CG50159-04) does not match the probe and primer sets Ag2059 and Ag2132. The probe and primer sets Ag2131 and Ag6048, described in Tables 51 and 50 are exclusive to NOV6c. These exclusions do not change the expression results or analyses presented below.

TABLE 43
Probe Name Ag1456

| Primers | Sequences | Length | Start | SEQ ID NO: |
|---|---|---|---|---|
| Forward | 5′-tcctgaggtgtggatgaatact | 22 | 91 | 195 |
| Probe | TET-5′-catcatctacaatggctaccccagtga-3′-TAMRA | 27 | 121 | 196 |
| Reverse | 5′-ccatcttcagtggtgacttcat-3′ | 22 | 153 | 197 |

TABLE 44
Probe Name Ag2446

| | | | | SEQ ID |
|---|---|---|---|---|
| Primers | Sequences | Length | Start Position | NO: |
| Forward | 5′-gaaacagtcggggaaacact-3′ | 20 | 354 | 198 |
| Probe | TET-5′-tggtcaagaagacacaaaacactctca-3′-TAMRA | 27 | 374 | 199 |
| Reverse | 5′-aaaccaaaggcccagaattt-3′ | 20 | 413 | 200 |

TABLE 45
Probe Name Ag2132

| Primers | Sequences | Length | Start Position | SEQ ID NO: |
|---|---|---|---|---|
| Forward | 5′-ggggaaatgacgctgataatat-3′ | 22 | 858 | 201 |
| Probe | TET-5′-cccctatatatgacctgactgccatg-3′-TAMRA | 26 | 903 | 202 |
| Reverse | 5′-cccaaatagcagtaggcacttt-3′ | 22 | 929 | 203 |

TABLE 46
Probe Name Ag2444

| | | | | SEQ ID |
|---|---|---|---|---|
| Primers | Sequences | Length | Start Position | NO: |
| Forward | 5′-gaaacagtcggggaaacact-3′ | 20 | 354 | 204 |
| Probe | TET-5′-tggtcaagaagacacaaaacactctca-3′-TAMRA | 27 | 374 | 205 |
| Reverse | 5′-aaaccaaaggcccagaattt-3′ | 20 | 413 | 206 |

TABLE 47
Probe Name Ag1899

| Primers | Sequences | Length | Start Position | SEQ ID NO: |
|---|---|---|---|---|
| Forward | 5′-tcctgaggtgtggatgaatact-3′ | 22 | 91 | 207 |
| Probe | TET-5′-catcatctacaatggctaccccagtga-3′-TAMRA | 27 | 121 | 208 |
| Reverse | 5′-ccatcttcagtggtgacttcat-3′ | 22 | 153 | 209 |

TABLE 48
Probe Name Ag2059

| Primers | Sequences | Length | Start Position | SEQ ID NO: |
|---|---|---|---|---|
| Forward | 5′-ggggaaatgacgctgataatat-3′ | 22 | 858 | 210 |
| Probe | TET-5′-cccctatatatgacctgactgccatg-3′-TAMRA | 26 | 903 | 211 |
| Reverse | 5′-cccaaatagcagtaggcacttt-3′ | 22 | 929 | 212 |

TABLE 49
Probe Name Ag2919

| Primers | Sequences | Length | Start Position | SEQ ID NO: |
|---|---|---|---|---|
| Forward | 5′-gaaatggcgctgataatatgaa-3′ | 22 | 861 | 213 |
| Probe | TET-5′-cccctatatatgacctgactgccatg-3′-TAMRA | 26 | 903 | 214 |
| Reverse | 5′-cccaaatagcagtaggcacttt-3′ | 22 | 929 | 215 |

TABLE 50
Probe Name Ag6048

| | | | Start | SEQ ID |
|---|---|---|---|---|
| Primers | Sequences Length | Position | NO: | |
| Forward | 5′-aattccataatcaaggctgtttt-3′ | 23 | 662 | 216 |
| Probe | TET-5′-tgcaacaataagatactctggttgatatgtagcga-3′-TAMRA | 35 | 743 | 217 |
| Reverse | 5′-ggggatgactctgattcatatttt-3′ | 24 | 810 | 218 |

TABLE 51
Probe Name Ag2131

| Primers | Sequences | Length | Start Position | SEQ ID NO: |
|---|---|---|---|---|
| Forward | 5′-tttatgtccttatgggctggat-3′ | 22 | 779 | 219 |
| Probe | TET-5′-cccctatatatgacctgactgccatg-3′-TAMRA | 26 | 831 | 220 |
| Reverse | 5′-cccaaatagcagtaggcacttt-3′ | 22 | 857 | 221 |

TABLE 52
AI_comprehensive panel_v1.0

| | Rel. | | |
|---|---|---|---|
| | Exp. (%) | | |
| | Ag1456, | | Rel. Exp. (%) |
| | Run | | Ag1456, Run |
| Tissue Name | 224501612 | Tissue Name | 224501612 |
| 110967 COPD-F | 0.0 | 112427 Match Control Psoriasis-F | 0.0 |
| 110980 COPD-F | 2.1 | 112418 Psoriasis-M | 0.0 |
| 110968 COPD-M | 0.0 | 112723 Match Control Psoriasis-M | 0.0 |
| 110977 COPD-M | 0.0 | 112419 Psoriasis-M | 0.0 |
| 110989 Emphysema-F | 2.6 | 112424 Match Control Psoriasis-M | 0.0 |
| 110992 Emphysema-F | 0.0 | 112420 Psoriasis-M | 4.4 |
| 110993 Emphysema-F | 0.0 | 112425 Match Control Psoriasis-M | 0.0 |
| 110994 Emphysema-F | 0.0 | 104689 (MF) OA Bone-Backus | 0.0 |
| 110995 Emphysema-F | 0.0 | 104690 (MF) Adj “Normal” Bone- | 3.0 |
| | | Backus | |
| 110996 Emphysema-F | 0.0 | 104691 (MF) OA Synovium-Backus | 35.1 |
| 110997 Asthma-M | 5.0 | 104692 (BA) OA Cartilage-Backus | 0.0 |
| 111001 Asthma-F | 1.6 | 104694 (BA) OA Bone-Backus | 3.2 |
| 111002 Asthma-F | 2.5 | 104695 (BA) Adj “Normal” Bone- | 3.1 |
| | | Backus | |
| 111003 Atopic Asthma-F | 0.0 | 104696 (BA) OA Synovium-Backus | 20.9 |
| 111004 Atopic Asthma-F | 0.0 | 104700 (SS) OA Bone-Backus | 39.0 |
| 111005 Atopic Asthma-F | 0.0 | 104701 (SS) Adj “Normal” Bone- | 3.3 |
| | | Backus | |
| 111006 Atopic Asthma-F | 0.0 | 104702 (SS) OA Synovium-Backus | 5.0 |
| 111417 Allergy-M | 0.0 | 117093 OA Cartilage Rep7 | 0.0 |
| 112347 Allergy-M | 0.8 | 112672 OA Bone5 | 0.0 |
| 112349 Normal Lung-F | 0.0 | 112673 OA Synovium5 | 0.0 |
| 112357 Normal Lung-F | 0.0 | 112674 OA Synovial Fluid cells5 | 0.0 |
| 112354 Normal Lung-M | 0.0 | 117100 OA Cartilage Rep14 | 0.0 |
| 112374 Crohns-F | 2.4 | 112756 OA Bone9 | 0.0 |
| 112389 Match Control Crohns-F | 100.0 | 112757 OA Synovium9 | 0.0 |
| 112375 Crohns-F | 0.0 | 112758 OA Synovial Fluid Cells9 | 1.3 |
| 112732 Match Control Crohns-F | 5.0 | 117125 RA Cartilage Rep2 | 0.0 |
| 112725 Crohns-M | 1.5 | 113492 Bone2 RA | 62.0 |
| 112387 Match Control Crohns-M | 0.0 | 113493 Synovium2 RA | 8.7 |
| 112378 Crohns-M | 0.0 | 113494 Syn Fluid Cells RA | 21.0 |
| 112390 Match Control Crohns-M | 2.3 | 113499 Cartilage4 RA | 20.6 |
| 112726 Crohns-M | 0.0 | 113500 Bone4 RA | 25.5 |
| 112731 Match Control Crohns-M | 0.0 | 113501 Synovium4 RA | 15.3 |
| 112380 Ulcer Col-F | 0.0 | 113502 Syn Fluid Cells4 RA | 8.5 |
| 112734 Match Control Ulcer Col-F | 52.5 | 113495 Cartilage3 RA | 33.7 |
| 112384 Ulcer Col-F | 0.0 | 1113496 Bone3 RA | 33.7 |
| 112737 Match Control Ulcer Col-F | 2.5 | 113497 Synovium3 RA | 19.9 |
| 112386 Ulcer Col-F | 2.4 | 113498 Syn Fluid Cells3 RA | 37.6 |
| 112738 Match Control Ulcer Col-F | 3.3 | 117106 Normal Cartilage Rep20 | 0.0 |
| 112381 Ulcer Col-M | 0.0 | 113663 Bone3 Normal | 0.0 |
| 112735 Match Control Ulcer Col-M | 1.4 | 113664 Synovium3 Normal | 0.9 |
| 112382 Ulcer Col-M | 28.5 | 113665 Syn Fluid Cells3 Normal | 0.0 |
| 112394 Match Control Ulcer Col-M | 0.0 | 117107 Normal Cartilage Rep22 | 2.4 |
| 112383 Ulcer Col-M | 0.0 | 113667 Bone4 Normal | 0.0 |
| 112736 Match Control Ulcer Col-M | 74.2 | 113668 Synovium4 Normal | 0.0 |
| 112423 Psoriasis-F | 4.4 | 113669 Syn Fluid Cells4 Normal | 0.0 |

TABLE 53
Panel 1.2

| | Rel. | | Rel. |
|---|---|---|---|
| | Exp. (%) | | Exp. (%) |
| | Ag1456, | | Ag1456, |
| | Run | | Run |
| Tissue Name | 138374123 | Tissue Name | 138374123 |
| Endothelial cells | 0.0 | Renal ca. 786-0 | 0.0 |
| Heart (Fetal) | 0.6 | Renal ca. A498 | 0.0 |
| Pancreas | 0.0 | Renal ca. RXF 393 | 0.0 |
| Pancreatic ca. CAPAN 2 | 0.0 | Renal ca. ACHN | 0.0 |
| Adrenal Gland | 10.7 | Renal ca. UO-31 | 0.0 |
| Thyroid | 1.3 | Renal ca. TK-10 | 0.0 |
| Salivary gland | 3.2 | Liver | 4.1 |
| Pituitary gland | 0.3 | Liver (fetal) | 4.5 |
| Brain (fetal) | 0.6 | Liver ca. (hepatoblast) HepG2 | 0.0 |
| Brain (whole) | 0.0 | Lung | 5.6 |
| Brain (amygdala) | 0.5 | Lung (fetal) | 1.2 |
| Brain (cerebellum) | 0.0 | Lung ca. (small cell) LX-1 | 5.9 |
| Brain (hippocampus) | 0.7 | Lung ca. (small cell) NCI-H69 | 1.7 |
| Brain (thalamus) | 0.7 | Lung ca. (s.cell var.) SHP-77 | 0.0 |
| Cerebral Cortex | 0.0 | Lung ca. (large cell)NCI-H460 | 0.0 |
| Spinal cord | 2.1 | Lung ca. (non-sm. cell) A549 | 0.0 |
| glio/astro U87-MG | 0.0 | Lung ca. (non-s.cell) NCI-H23 | 60.3 |
| glio/astro U-118-MG | 1.8 | Lung ca. (non-s.cell) HOP-62 | 0.0 |
| astrocytoma SW1783 | 0.0 | Lung ca. (non-s.cl) NCI-H522 | 2.8 |
| Neuro*; met SK-N-AS | 0.0 | Lung ca. (squam.) SW 900 | 0.0 |
| astrocytoma SF-539 | 0.0 | Lung ca. (squam.) NCI-H596 | 0.0 |
| astrocytoma SNB-75 | 0.0 | Mammary gland | 0.0 |
| glioma SNB-19 | 0.0 | Breast ca.* (pl.ef) MCF-7 | 0.9 |
| glioma U251 | 0.0 | Breast ca.* (pl.ef) MDA-MB-231 | 0.0 |
| glioma SF-295 | 0.0 | Breast ca.* (pl.ef) T47D | 0.0 |
| Heart | 19.9 | Breast ca. BT-549 | 0.0 |
| Skeletal Muscle | 8.2 | Breast ca. MDA-N | 0.0 |
| Bone marrow | 100.0 | Ovary | 0.0 |
| Thymus | 0.6 | Ovarian ca. OVCAR-3 | 0.0 |
| Spleen | 12.3 | Ovarian ca. OVCAR-4 | 0.0 |
| Lymph node | 0.9 | Ovarian ca. OVCAR-5 | 1.4 |
| Colorectal Tissue | 1.9 | Ovarian ca. OVCAR-8 | 0.0 |
| Stomach | 2.0 | Ovarian ca. IGROV-1 | 0.0 |
| Small intestine | 1.2 | Ovarian ca. (ascites) SK-OV-3 | 0.0 |
| Colon ca. SW480 | 0.5 | Uterus | 0.4 |
| Colon ca.* SW620 (SW480 met) | 3.1 | Placenta | 2.2 |
| Colon ca. HT29 | 0.0 | Prostate | 0.0 |
| Colon ca. HCT-116 | 0.0 | Prostate ca.* (bone met) PC-3 | 0.0 |
| Colon ca. CaCo-2 | 0.5 | Testis | 0.0 |
| Colon ca. Tissue (ODO3866) | 8.2 | Melanoma Hs688(A).T | 0.0 |
| Colon ca. HCC-2998 | 0.0 | Melanoma* (met) Hs688(B).T | 0.0 |
| Gastric ca.* (liver met) NCI-N87 | 2.4 | Melanoma UACC-62 | 0.0 |
| Bladder | 29.1 | Melanoma M14 | 0.0 |
| Trachea | 0.6 | Melanoma LOX IMVI | 0.0 |
| Kidney | 3.1 | Melanoma* (met) SK-MEL-5 | 1.2 |
| Kidney (fetal) | 2.5 | | |

TABLE 54
Panel 1.3D

| | | | | Rel. Exp. (%) |
|---|---|---|---|---|
| | Rel. Exp. (%) | Rel. Exp. (%) | Rel. Exp. (%) | Ag2444, |
| | Ag1456, Run | Ag1456, Run | Ag2132, Run | Run |
| Tissue Name | 147644869 | 165529464 | 160164823 | 165629988 |
| Liver adenocarcinoma | 0.0 | 0.0 | 0.0 | 0.0 |
| Pancreas | 0.0 | 0.0 | 0.0 | 1.9 |
| Pancreatic ca. CAPAN 2 | 0.0 | 0.0 | 0.0 | 0.0 |
| Adrenal gland | 9.2 | 7.6 | 5.2 | 1.9 |
| Thyroid | 0.0 | 0.0 | 0.0 | 1.6 |
| Salivary gland | 0.0 | 0.0 | 0.0 | 0.4 |
| Pituitary gland | 0.0 | 0.0 | 0.0 | 0.6 |
| Brain (fetal) | 0.0 | 0.0 | 0.0 | 1.4 |
| Brain (whole) | 0.0 | 0.0 | 0.0 | 0.3 |
| Brain (amygdala) | 0.0 | 0.0 | 0.0 | 0.0 |
| Brain (cerebellum) | 0.0 | 0.0 | 0.0 | 0.0 |
| Brain (hippocampus) | 0.0 | 0.0 | 0.0 | 0.4 |
| Brain (substantia nigra) | 4.6 | 0.0 | 0.0 | 0.4 |
| Brain (thalamus) | 0.0 | 0.0 | 0.0 | 0.0 |
| Cerebral Cortex | 0.0 | 0.0 | 0.0 | 0.5 |
| Spinal cord | 0.0 | 10.4 | 3.5 | 1.2 |
| glio/astro U87-MG | 0.0 | 0.0 | 0.0 | 0.0 |
| glio/astro U-118-MG | 12.4 | 0.0 | 10.7 | 8.5 |
| astrocytoma SW1783 | 0.0 | 0.0 | 0.0 | 0.0 |
| Neuro*; met SK-N-AS | 0.0 | 0.0 | 0.0 | 0.0 |
| astrocytoma SF-539 | 0.0 | 0.0 | 0.0 | 0.0 |
| astrocytoma SNB-75 | 0.0 | 0.0 | 0.0 | 2.5 |
| glioma SNB-19 | 0.0 | 0.0 | 0.0 | 0.0 |
| glioma U251 | 0.0 | 0.0 | 0.0 | 0.6 |
| glioma SF-295 | 0.0 | 0.0 | 0.0 | 0.0 |
| Heart (fetal) | 5.8 | 0.0 | 0.0 | 0.0 |
| Heart | 0.0 | 0.0 | 0.0 | 0.5 |
| Skeletal muscle (fetal) | 0.0 | 0.0 | 0.0 | 0.3 |
| Skeletal muscle | 0.0 | 6.2 | 5.0 | 0.6 |
| Bone marrow | 100.0 | 100.0 | 66.4 | 0.0 |
| Thymus | 0.0 | 0.0 | 7.2 | 0.0 |
| Spleen | 11.4 | 8.8 | 21.2 | 0.0 |
| Lymph node | 5.0 | 7.4 | 0.0 | 1.3 |
| Colorectal | 0.0 | 0.0 | 0.0 | 0.3 |
| Stomach | 0.0 | 0.0 | 0.0 | 0.9 |
| Small intestine | 0.0 | 0.0 | 0.0 | 0.4 |
| Colon ca. SW480 | 0.0 | 0.0 | 0.0 | 0.0 |
| Colon ca.* SW620(SW480 met) | 0.0 | 0.0 | 0.0 | 0.0 |
| Colon ca. HT29 | 0.0 | 0.0 | 0.0 | 1.1 |
| Colon ca. HCT-116 | 0.0 | 0.0 | 0.0 | 0.0 |
| Colon ca. CaCo-2 | 0.0 | 0.0 | 0.0 | 0.8 |
| Colon ca. tissue(ODO3866) | 10.8 | 17.3 | 23.2 | 0.6 |
| Colon ca. HCC-2998 | 0.0 | 0.0 | 0.0 | 1.4 |
| Gastric ca.* (liver met) NCI-N87 | 0.0 | 0.0 | 1.8 | 100.0 |
| Bladder | 0.0 | 6.7 | 0.0 | 1.5 |
| Trachea | 0.0 | 0.0 | 31.6 | 1.2 |
| Kidney | 0.0 | 0.0 | 0.0 | 0.6 |
| Kidney (fetal) | 5.1 | 0.0 | 0.0 | 0.0 |
| Renal ca. 786-0 | 0.0 | 0.0 | 0.0 | 0.0 |
| Renal ca. A498 | 0.0 | 0.0 | 3.9 | 0.1 |
| Renal ca. RXF 393 | 0.0 | 0.0 | 0.0 | 1.4 |
| Renal ca. ACHN | 0.0 | 0.0 | 0.0 | 24.7 |
| Renal ca. UO-31 | 0.0 | 0.0 | 0.0 | 0.0 |
| Renal ca. TK-10 | 0.0 | 0.0 | 0.0 | 0.0 |
| Liver | 0.0 | 0.0 | 0.0 | 0.0 |
| Liver (fetal) | 3.7 | 0.0 | 0.0 | 0.0 |
| Liver ca. (hepatoblast) HepG2 | 0.0 | 0.0 | 0.0 | 0.0 |
| Lung | 38.4 | 25.0 | 100.0 | 1.3 |
| Lung (fetal) | 18.9 | 5.7 | 15.1 | 0.0 |
| Lung ca. (small cell) LX-1 | 11.7 | 0.0 | 0.0 | 0.3 |
| Lung ca. (small cell) NCI-H69 | 0.0 | 0.0 | 0.0 | 2.3 |
| Lung ca. (s.cell var.) SHP-77 | 0.0 | 0.0 | 0.0 | 0.0 |
| Lung ca. (large cell) NCI-H460 | 0.0 | 0.0 | 0.0 | 0.5 |
| Lung ca. (non-sm.cell) A549 | 0.0 | 0.0 | 0.0 | 3.3 |
| Lung ca. (non-s.cell) NCI-H23 | 38.2 | 17.9 | 10.2 | 21.5 |
| Lung ca. (non-s.cell) HOP-62 | 0.0 | 0.0 | 0.0 | 0.0 |
| Lung ca. (non-s.cl) NCI-H522 | 0.0 | 0.0 | 0.0 | 0.3 |
| Lung ca. (squam.) SW 900 | 0.0 | 0.0 | 0.0 | 2.2 |
| Lung ca. (squam.) NCI-H596 | 0.0 | 0.0 | 0.0 | 0.5 |
| Mammary gland | 0.0 | 0.0 | 0.0 | 0.6 |
| Breast ca.* (pl.ef) MCF-7 | 0.0 | 0.0 | 0.0 | 35.4 |
| Breast ca.* (pl.ef) MDA-MB-231 | 0.0 | 0.0 | 0.0 | 0.0 |
| Breast ca.* (pl.ef) T47D | 0.0 | 0.0 | 0.0 | 5.6 |
| Breast ca. BT-549 | 0.0 | 0.0 | 0.0 | 1.7 |
| Breast ca. MDA-N | 0.0 | 0.0 | 0.0 | 0.0 |
| Ovary | 0.0 | 0.0 | 0.0 | 2.3 |
| Ovarian ca. OVCAR-3 | 0.0 | 0.0 | 0.0 | 17.7 |
| Ovarian ca. OVCAR-4 | 0.0 | 0.0 | 0.0 | 17.1 |
| Ovarian ca. OVCAR-5 | 0.0 | 0.0 | 0.0 | 0.9 |
| Ovarian ca. OVCAR-8 | 0.0 | 0.0 | 0.0 | 4.4 |
| Ovarian ca. IGROV-1 | 0.0 | 0.0 | 0.0 | 0.0 |
| Ovarian ca.* (ascites) SK-OV-3 | 0.0 | 0.0 | 0.0 | 8.0 |
| Uterus | 0.0 | 0.0 | 0.0 | 3.0 |
| Placenta | 5.3 | 0.0 | 16.5 | 0.0 |
| Prostate | 0.0 | 0.0 | 0.0 | 0.0 |
| Prostate ca.* (bone met)PC-3 | 0.0 | 0.0 | 0.0 | 32.8 |
| Testis | 5.3 | 0.0 | 0.0 | 1.3 |
| Melanoma Hs688(A).T | 0.0 | 0.0 | 0.0 | 0.0 |
| Melanoma* (met) Hs688(B).T | 0.0 | 0.0 | 0.0 | 0.0 |
| Melanoma UACC-62 | 0.0 | 0.0 | 0.0 | 0.5 |
| Melanoma M14 | 0.0 | 0.0 | 0.0 | 0.6 |
| Melanoma LOX IMVI | 0.0 | 0.0 | 0.0 | 0.0 |
| Melanoma* (met) SK-MEL-5 | 0.0 | 0.0 | 0.0 | 0.0 |
| Adipose | 27.0 | 14.3 | 10.7 | 4.0 |

TABLE 55
Panel 2D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1456, | Ag1456, | Ag1456, | | Ag1456, | Ag1456, | Ag1456, |
| | Run | Run | Run | Tissue | Run | Run | Run |
| Tissue Name | 147644930 | 148059395 | 162599938 | Name | 147644930 | 148059395 | 162599938 |
| Normal | 13.2 | 2.1 | 6.3 | Kidney | 0.0 | 0.6 | 1.0 |
| Colon | | | | Margin | | | |
| | | | | 8120608 | | | |
| CC Well to | 5.5 | 2.4 | 2.6 | Kidney | 1.0 | 0.8 | 0.8 |
| Mod Diff | | | | Cancer | | | |
| (ODO3866) | | | | 8120613 | | | |
| CC Margin | 2.1 | 3.2 | 2.3 | Kidney | 0.0 | 0.0 | 0.0 |
| (ODO3866) | | | | Margin | | | |
| | | | | 8120614 | | | |
| CC Gr. 2 | 0.6 | 0.0 | 1.7 | Kidney | 17.9 | 13.8 | 15.0 |
| rectosigmoid | | | | Cancer | | | |
| (ODO3868) | | | | 9010320 | | | |
| CC Margin | 0.0 | 0.0 | 0.8 | Kidney | 0.7 | 1.4 | 1.4 |
| (ODO3868) | | | | Margin | | | |
| | | | | 9010321 | | | |
| CC Mod Diff | 1.8 | 2.9 | 3.5 | Normal | 0.0 | 0.0 | 0.0 |
| (ODO3920) | | | | Uterus | | | |
| CC Margin | 0.5 | 1.2 | 2.6 | Uterus | 1.2 | 0.5 | 2.1 |
| (ODO3920) | | | | Cancer | | | |
| | | | | 064011 | | | |
| CC Gr. 2 | 1.3 | 9.2 | 6.5 | Normal | 0.0 | 0.6 | 0.7 |
| ascend colon | | | | Thyroid | | | |
| (ODO3921) | | | | | | | |
| CC Margin | 0.0 | 0.5 | 1.7 | Thyroid | 0.0 | 1.3 | 2.8 |
| (ODO3921) | | | | Cancer | | | |
| | | | | 064010 | | | |
| CC from | 2.3 | 6.7 | 7.1 | Thyroid | 1.9 | 0.6 | 3.0 |
| Partial | | | | Cancer | | | |
| Hepatectomy | | | | A302152 | | | |
| (ODO4309) | | | | | | | |
| Mets | | | | | | | |
| Liver Margin | 3.2 | 7.3 | 2.3 | Thyroid | 0.0 | 0.0 | 1.9 |
| (ODO4309) | | | | Margin | | | |
| | | | | A302153 | | | |
| Colon mets to | 1.3 | 0.6 | 0.0 | Normal | 0.8 | 1.9 | 0.0 |
| lung | | | | Breast | | | |
| (OD04451- | | | | | | | |
| 01) | | | | | | | |
| Lung Margin | 2.0 | 4.5 | 1.9 | Breast | 0.0 | 0.0 | 0.0 |
| (OD04451- | | | | Cancer | | | |
| 02) | | | | (OD04566) | | | |
| Normal | 0.0 | 0.0 | 0.0 | Breast | 0.0 | 1.9 | 0.0 |
| Prostate | | | | Cancer | | | |
| 6546-1 | | | | (OD04590- | | | |
| | | | | 01) | | | |
| Prostate | 0.7 | 0.0 | 2.9 | Breast | 0.9 | 0.5 | 1.4 |
| Cancer | | | | Cancer | | | |
| (OD04410) | | | | Mets | | | |
| | | | | (OD04590- | | | |
| | | | | 03) | | | |
| Prostate | 0.6 | 0.0 | 0.0 | Breast | 1.1 | 0.6 | 1.7 |
| Margin | | | | Cancer | | | |
| (OD04410) | | | | Metastasis | | | |
| | | | | (OD04655- | | | |
| | | | | 05) | | | |
| Prostate | 0.6 | 0.0 | 0.0 | Breast | 0.0 | 0.7 | 0.0 |
| Cancer | | | | Cancer | | | |
| (OD04720- | | | | 064006 | | | |
| 01) | | | | | | | |
| Prostate | 2.8 | 0.2 | 2.9 | Breast | 0.7 | 0.0 | 0.9 |
| Margin | | | | Cancer | | | |
| (OD04720- | | | | 1024 | | | |
| 02) | | | | | | | |
| Normal Lung | 7.4 | 8.2 | 0.0 | Breast | 0.0 | 0.0 | 0.0 |
| 061010 | | | | Cancer | | | |
| | | | | 9100266 | | | |
| Lung Met to | 6.1 | 2.0 | 5.8 | Breast | 0.7 | 0.0 | 0.0 |
| Muscle | | | | Margin | | | |
| (ODO4286) | | | | 9100265 | | | |
| Muscle | 1.5 | 0.6 | 1.1 | Breast | 0.8 | 0.0 | 0.0 |
| Margin | | | | Cancer | | | |
| (ODO4286) | | | | A209073 | | | |
| Lung | 9.9 | 7.3 | 4.1 | Breast | 0.0 | 0.0 | 0.0 |
| Malignant | | | | Margin | | | |
| Cancer | | | | A2090734 | | | |
| (OD03126) | | | | | | | |
| Lung Margin | 33.9 | 28.1 | 27.0 | Normal | 0.0 | 0.0 | 1.1 |
| (OD03126) | | | | Liver | | | |
| Lung Cancer | 13.3 | 11.2 | 13.0 | Liver | 1.4 | 0.0 | 0.0 |
| (OD04404) | | | | Cancer | | | |
| | | | | 064003 | | | |
| Lung Margin | 32.8 | 22.2 | 28.3 | Liver | 0.0 | 0.0 | 0.8 |
| (OD04404) | | | | Cancer | | | |
| | | | | 1025 | | | |
| Lung Cancer | 4.5 | 1.3 | 5.7 | Liver | 2.2 | 1.8 | 0.9 |
| (OD04565) | | | | Cancer | | | |
| | | | | 1026 | | | |
| Lung Margin | 0.0 | 7.2 | 4.9 | Liver | 1.2 | 1.0 | 0.0 |
| (OD04565) | | | | Cancer | | | |
| | | | | 6004-T | | | |
| Lung Cancer | 2.1 | 1.6 | 3.5 | Liver | 1.1 | 0.7 | 2.7 |
| (OD04237- | | | | Tissue | | | |
| 01) | | | | 6004-N | | | |
| Lung Margin | 100.0 | 100.0 | 100.0 | Liver | 0.0 | 0.0 | 0.8 |
| (OD04237- | | | | Cancer | | | |
| 02) | | | | 6005-T | | | |
| Ocular Mel | 0.3 | 0.0 | 0.0 | Liver | 0.0 | 0.0 | 0.6 |
| Met to Liver | | | | Tissue | | | |
| (ODO4310) | | | | 6005-N | | | |
| Liver Margin | 1.9 | 0.6 | 0.7 | Normal | 3.9 | 1.8 | 8.4 |
| (ODO4310) | | | | Bladder | | | |
| Melanoma | 0.5 | 0.0 | 0.0 | Bladder | 0.0 | 0.0 | 0.0 |
| Mets to Lung | | | | Cancer | | | |
| (OD04321) | | | | 1023 | | | |
| Lung Margin | 22.8 | 27.5 | 24.5 | Bladder | 3.3 | 5.2 | 1.7 |
| (OD04321) | | | | Cancer | | | |
| | | | | A302173 | | | |
| Normal | 0.0 | 0.6 | 1.6 | Bladder | 13.0 | 11.0 | 11.8 |
| Kidney | | | | Cancer | | | |
| | | | | (OD04718- | | | |
| | | | | 01) | | | |
| Kidney Ca, | 8.7 | 11.5 | 16.5 | Bladder | 14.6 | 12.7 | 15.9 |
| Nuclear grade | | | | Normal | | | |
| 2 (OD04338) | | | | Adjacent | | | |
| | | | | (OD04718- | | | |
| | | | | 03) | | | |
| Kidney | 2.0 | 6.1 | 3.2 | Normal | 0.0 | 0.0 | 0.0 |
| Margin | | | | Ovary | | | |
| (OD04338) | | | | | | | |
| Kidney Ca | 1.4 | 0.6 | 0.8 | Ovarian | 0.0 | 0.8 | 0.0 |
| Nuclear grade | | | | Cancer | | | |
| 1/2 | | | | 064008 | | | |
| (OD04339) | | | | | | | |
| Kidney | 0.0 | 0.5 | 2.6 | Ovarian | 2.9 | 2.3 | 6.0 |
| Margin | | | | Cancer | | | |
| (OD04339) | | | | (OD04768- | | | |
| | | | | 07) | | | |
| Kidney Ca, | 20.0 | 26.8 | 25.9 | Ovary | 16.7 | 20.9 | 12.9 |
| Clear cell | | | | Margin | | | |
| type | | | | (OD04768- | | | |
| (OD04340) | | | | 08) | | | |
| Kidney | 7.2 | 3.4 | 9.7 | Normal | 1.1 | 3.3 | 3.2 |
| Margin | | | | Stomach | | | |
| (OD04340) | | | | | | | |
| Kidney Ca, | 0.7 | 0.0 | 0.5 | Gastric | 0.0 | 0.0 | 0.0 |
| Nuclear grade | | | | Cancer | | | |
| 3 (OD04348) | | | | 9060358 | | | |
| Kidney | 1.2 | 1.4 | 1.8 | Stomach | 3.1 | 5.9 | 3.3 |
| Margin | | | | Margin | | | |
| (OD04348) | | | | 9060359 | | | |
| Kidney | 11.2 | 11.2 | 20.9 | Gastric | 13.2 | 3.7 | 11.0 |
| Cancer | | | | Cancer | | | |
| (OD04622- | | | | 9060395 | | | |
| 01) | | | | | | | |
| Kidney | 1.6 | 1.0 | 1.4 | Stomach | 1.6 | 2.7 | 4.3 |
| Margin | | | | Margin | | | |
| (OD04622- | | | | 9060394 | | | |
| 03) | | | | | | | |
| Kidney | 0.7 | 0.0 | 0.0 | Gastric | 19.1 | 7.4 | 9.8 |
| Cancer | | | | Cancer | | | |
| (OD04450- | | | | 9060397 | | | |
| 01) | | | | | | | |
| Kidney | 0.0 | 1.4 | 3.2 | Stomach | 0.0 | 1.2 | 0.8 |
| Margin | | | | Margin | | | |
| (OD04450- | | | | 9060396 | | | |
| 03) | | | | | | | |
| Kidney | 0.0 | 0.0 | 0.0 | Gastric | 4.3 | 5.6 | 3.9 |
| Cancer | | | | Cancer | | | |
| 8120607 | | | | 064005 | | | |

TABLE 56
Panel 4D

| | Rel. | Rel. | Rel. | Rel. | Rel. | Rel. |
|---|---|---|---|---|---|---|
| | Exp. (%) | Exp. (%) | Exp. (%) | Exp. (%) | Exp. (%) | Exp. (%) |
| | Ag1456, | Ag1456, | Ag1899, | Ag2059, | Ag2132, | Ag2444, |
| | Run | Run | Run | Run | Run | Run |
| Tissue Name | 139309823 | 144691235 | 165870453 | 161426290 | 159366502 | 164320874 |
| Secondary Th1 act | 0.0 | 0.0 | 0.0 | 0.0 | 0.0 | 0.0 |
| Secondary Th2 act | 0.4 | 0.4 | 0.0 | 0.0 | 0.0 | 0.0 |
| Secondary Tr1 act | 0.0 | 0.0 | 0.0 | 0.0 | 0.0 | 0.0 |
| Secondary Th1 rest | 0.0 | 0.0 | 0.3 | 0.0 | 0.0 | 0.0 |
| Secondary Th2 rest | 6.1 | 4.8 | 2.4 | 0.8 | 2.7 | 0.0 |
| Secondary Tr1 rest | 0.4 | 0.0 | 0.3 | 0.0 | 1.4 | 0.0 |
| Primary Th1 act | 0.0 | 0.7 | 0.0 | 0.0 | 0.0 | 0.0 |
| Primary Th2 act | 1.5 | 0.3 | 0.6 | 0.0 | 0.0 | 0.0 |
| Primary Tr1 act | 0.0 | 0.6 | 0.1 | 0.0 | 0.0 | 0.0 |
| Primary Th1 rest | 4.5 | 4.1 | 7.9 | 3.0 | 5.3 | 0.0 |
| Primary Th2 rest | 6.5 | 2.9 | 3.7 | 6.3 | 1.1 | 41.5 |
| Primary Tr1 rest | 2.7 | 3.5 | 1.6 | 2.5 | 1.0 | 0.0 |
| CD45RA CD4 | 0.0 | 0.0 | 0.0 | 0.0 | 0.0 | 0.0 |
| lymphocyte act | | | | | | |
| CD45RO CD4 | 0.0 | 0.4 | 0.3 | 0.0 | 0.0 | 0.0 |
| lymphocyte act | | | | | | |
| CD8 lymphocyte act | 0.0 | 0.0 | 0.0 | 0.0 | 0.0 | 0.0 |
| Secondary CD8 | 0.5 | 0.0 | 0.2 | 0.0 | 0.0 | 0.0 |
| lymphocyte rest | | | | | | |
| Secondary CD8 | 0.6 | 0.0 | 0.0 | 0.0 | 0.0 | 0.0 |
| lymphocyte act | | | | | | |
| CD4 lymphocyte | 3.1 | 1.1 | 1.4 | 5.1 | 0.0 | 0.0 |
| none | | | | | | |
| 2ry | 4.3 | 5.9 | 4.7 | 2.1 | 3.5 | 0.0 |
| Th1/Th2/Tr1_anti- | | | | | | |
| CD95 CH11 | | | | | | |
| LAK cells rest | 0.5 | 1.1 | 0.5 | 0.0 | 0.0 | 0.0 |
| LAK cells IL-2 | 1.0 | 1.4 | 0.8 | 0.0 | 1.6 | 0.0 |
| LAK cells IL-2 + IL- | 1.0 | 0.9 | 0.2 | 0.0 | 0.0 | 0.0 |
| 12 | | | | | | |
| LAK cells IL-2 + IFN | 0.5 | 2.1 | 0.6 | 0.0 | 0.0 | 0.0 |
| gamma | | | | | | |
| LAK cells IL-2 + IL- | 1.0 | 0.4 | 0.4 | 0.0 | 0.0 | 0.0 |
| 18 | | | | | | |
| LAK cells | 17.1 | 17.8 | 8.0 | 8.5 | 10.0 | 0.0 |
| PMA/ionomycin | | | | | | |
| NK Cells IL-2 rest | 0.0 | 0.0 | 0.2 | 1.2 | 0.0 | 0.0 |
| Two Way MLR 3 | 0.0 | 0.0 | 0.0 | 1.5 | 0.0 | 38.7 |
| day | | | | | | |
| Two Way MLR 5 | 0.0 | 0.3 | 0.0 | 0.0 | 0.0 | 0.0 |
| day | | | | | | |
| Two Way MLR 7 | 0.0 | 0.5 | 0.0 | 0.0 | 0.0 | 0.0 |
| day | | | | | | |
| PBMC rest | 20.3 | 22.2 | 18.4 | 6.7 | 14.0 | 100.0 |
| PBMC PWM | 0.5 | 0.0 | 0.0 | 0.0 | 1.3 | 45.7 |
| PBMC PHA-L | 0.0 | 1.0 | 0.2 | 0.0 | 0.0 | 0.0 |
| Ramos (B cell) none | 36.1 | 48.6 | 21.0 | 0.0 | 7.2 | 44.1 |
| Ramos (B cell) | 100.0 | 87.1 | 16.6 | 44.1 | 27.9 | 46.7 |
| ionomycin | | | | | | |
| B lymphocytes | 0.5 | 0.0 | 0.0 | 1.6 | 0.0 | 0.0 |
| PWM | | | | | | |
| B lymphocytes | 0.5 | 0.0 | 0.0 | 0.0 | 0.0 | 0.0 |
| CD40L and IL-4 | | | | | | |
| EOL-1 dbcAMP | 0.0 | 0.0 | 0.2 | 0.0 | 0.0 | 0.0 |
| EOL-1 dbcAMP | 0.4 | 0.0 | 0.6 | 1.1 | 1.2 | 0.0 |
| PMA/ionomycin | | | | | | |
| Dendritic cells none | 5.6 | 4.7 | 4.3 | 3.7 | 8.4 | 0.0 |
| Dendritic cells LPS | 3.0 | 1.8 | 2.3 | 3.7 | 1.8 | 30.1 |
| Dendritic cells anti- | 2.6 | 3.2 | 2.0 | 4.7 | 0.0 | 0.0 |
| CD40 | | | | | | |
| Monocytes rest | 97.3 | 100.0 | 100.0 | 100.0 | 100.0 | 82.4 |
| Monocytes LPS | 34.2 | 34.4 | 20.3 | 15.8 | 19.3 | 32.5 |
| Macrophages rest | 5.1 | 5.5 | 3.0 | 4.0 | 1.3 | 0.0 |
| Macrophages LPS | 7.5 | 9.7 | 4.8 | 3.0 | 0.0 | 0.0 |
| HUVEC none | 0.0 | 0.0 | 0.0 | 0.0 | 0.0 | 0.0 |
| HUVEC starved | 0.0 | 0.0 | 0.0 | 0.0 | 0.0 | 0.0 |
| HUVEC IL-1beta | 0.0 | 0.0 | 0.0 | 0.0 | 0.0 | 0.0 |
| HUVEC IFN | 0.0 | 0.0 | 0.0 | 0.0 | 0.0 | 0.0 |
| gamma | | | | | | |
| HUVEC TNF | 0.0 | 0.0 | 0.0 | 0.0 | 0.0 | 0.0 |
| alpha + IFN gamma | | | | | | |
| HUVEC TNF | 0.0 | 0.0 | 0.0 | 0.0 | 0.0 | 0.0 |
| alpha + IL4 | | | | | | |
| HUVEC IL-11 | 0.0 | 0.0 | 0.0 | 0.0 | 0.0 | 0.0 |
| Lung Microvascular | 0.0 | 0.0 | 0.0 | 0.0 | 0.0 | 0.0 |
| EC none | | | | | | |
| Lung Microvascular | 0.0 | 0.0 | 0.0 | 0.0 | 0.0 | 0.0 |
| EC TNFalpha + IL- | | | | | | |
| 1beta | | | | | | |
| Microvascular | 0.0 | 0.0 | 0.0 | 0.0 | 0.0 | 0.0 |
| Dermal EC none | | | | | | |
| Microsvasular | 0.0 | 0.0 | 0.0 | 0.0 | 0.0 | 0.0 |
| Dermal EC | | | | | | |
| TNFalpha + IL- | | | | | | |
| 1beta | | | | | | |
| Bronchial | 0.0 | 0.0 | 0.0 | 0.0 | 0.0 | 0.0 |
| Epithelium | | | | | | |
| TNFalpha + IL1beta | | | | | | |
| Small airway | 0.5 | 0.5 | 0.5 | 0.0 | 0.0 | 0.0 |
| epithelium none | | | | | | |
| Small airway | 4.0 | 3.8 | 2.1 | 6.2 | 6.3 | 0.0 |
| epithelium | | | | | | |
| TNFalpha + IL- | | | | | | |
| 1beta | | | | | | |
| Coronery artery | 0.0 | 0.0 | 0.0 | 0.0 | 0.0 | 0.0 |
| SMC rest | | | | | | |
| Coronery artery | 0.0 | 0.0 | 0.0 | 0.0 | 0.0 | 0.0 |
| SMC TNFalpha + | | | | | | |
| IL-1beta | | | | | | |
| Astrocytes rest | 0.0 | 0.0 | 0.0 | 0.0 | 0.0 | 0.0 |
| Astrocytes | 0.0 | 0.0 | 0.0 | 0.0 | 0.0 | 0.0 |
| TNFalpha + IL- | | | | | | |
| 1beta | | | | | | |
| KU-812 (Basophil) | 0.0 | 0.0 | 0.0 | 0.0 | 0.0 | 0.0 |
| rest | | | | | | |
| KU-812 (Basophil) | 0.0 | 0.0 | 0.0 | 0.0 | 0.0 | 0.0 |
| PMA/ionomycin | | | | | | |
| CCD1106 | 0.0 | 0.0 | 0.0 | 0.0 | 0.0 | 0.0 |
| (Keratinocytes) | | | | | | |
| none | | | | | | |
| CCD1106 | 0.0 | 0.4 | 0.2 | 0.0 | 0.0 | 0.0 |
| (Keratinocytes) | | | | | | |
| TNFalpha + IL- | | | | | | |
| 1beta | | | | | | |
| Liver cirrhosis | 5.4 | 5.4 | 6.9 | 3.0 | 1.4 | 0.0 |
| Lupus kidney | 0.4 | 0.4 | 0.9 | 0.0 | 0.0 | 0.0 |
| NCI-H292 none | 0.0 | 0.4 | 0.0 | 0.0 | 1.5 | 0.0 |
| NCI-H292 IL-4 | 0.0 | 0.0 | 0.0 | 0.0 | 0.0 | 0.0 |
| NCI-H292 IL-9 | 0.0 | 0.0 | 0.3 | 0.0 | 0.0 | 0.0 |
| NCI-H292 IL-13 | 0.0 | 0.0 | 0.0 | 0.0 | 0.0 | 0.0 |
| NCI-H292 IFN | 0.0 | 0.0 | 0.0 | 0.0 | 0.0 | 0.0 |
| gamma | | | | | | |
| HPAEC none | 0.0 | 0.0 | 0.0 | 0.0 | 0.0 | 0.0 |
| HPAEC TNF | 0.0 | 0.0 | 0.0 | 0.0 | 0.0 | 0.0 |
| alpha + IL-1 beta | | | | | | |
| Lung fibroblast | 0.0 | 0.0 | 0.0 | 0.0 | 0.0 | 0.0 |
| none | | | | | | |
| Lung fibroblast TNF | 0.0 | 0.0 | 0.0 | 0.0 | 0.0 | 27.0 |
| alpha + IL-1 beta | | | | | | |
| Lung fibroblast IL-4 | 0.0 | 0.0 | 0.0 | 0.0 | 0.0 | 0.0 |
| Lung fibroblast IL-9 | 0.0 | 0.0 | 0.0 | 0.0 | 0.0 | 0.0 |
| Lung fibroblast IL- | 0.0 | 0.0 | 0.0 | 0.0 | 0.0 | 0.0 |
| 13 | | | | | | |
| Lung fibroblast IFN | 0.0 | 0.0 | 0.0 | 0.0 | 0.0 | 0.0 |
| gamma | | | | | | |
| Dermal fibroblast | 0.0 | 0.0 | 0.0 | 0.0 | 0.0 | 0.0 |
| CCD1070 rest | | | | | | |
| Dermal fibroblast | 1.6 | 0.0 | 0.2 | 0.0 | 0.0 | 0.0 |
| CCD1070 TNF | | | | | | |
| alpha | | | | | | |
| Dermal fibroblast | 0.0 | 0.0 | 0.0 | 0.0 | 0.0 | 0.0 |
| CCD1070 IL-1 beta | | | | | | |
| Dermal fibroblast | 0.0 | 0.0 | 0.1 | 0.0 | 0.0 | 0.0 |
| IFN gamma | | | | | | |
| Dermal fibroblast | 0.5 | 0.0 | 0.0 | 0.0 | 0.0 | 0.0 |
| IL-4 | | | | | | |
| IBD Colitis 2 | 0.6 | 0.0 | 1.4 | 0.0 | 0.0 | 0.0 |
| IBD Crohn's | 1.4 | 1.5 | 2.0 | 0.0 | 0.0 | 0.0 |
| Colon | 0.6 | 0.0 | 0.6 | 0.0 | 3.1 | 0.0 |
| Lung | 3.7 | 5.2 | 1.5 | 2.1 | 4.9 | 0.0 |
| Thymus | 0.5 | 0.0 | 0.2 | 0.0 | 0.0 | 0.0 |
| Kidney | 2.6 | 4.4 | 0.6 | 1.6 | 0.0 | 0.0 |

AI_comprehensive panel_v1.0 Summary: Ag 1456 Highest expression of the NOV6a transcript is found in normal colon tissue adjacent to tissue affected by Crohn's or ulcerative colitis (CTs=33). This transcript is also found in normal colon on panels 1.2 and 2D. Since the NOV6a transcript appears to be down regulated in diseased colon, therapeutic modulation of the expression or function of the this gene or its protein product, through the use protein therapeutics, could regulate normal homeostasis of this tissue and be beneficial for the treatment of inflammatory bowel diseases.

CNS_neurodegeneration_v1.0 Summary: Ag2446 Expression of the NOV6a gene is low/undetectable in all samples on this panel. (CTs>35). The amp plot indicates that there may have been a probe failure in this experiment. (Data not shown.)

Panel 1.2 Summary: Ag1456 Highest expression of the NOV6a gene is detected in bone marrow (CT=28.9). Furthermore, the difference in expression between heart (CT=31.2) and fetal heart tissue (CT=36.2) is significant in this panel. Thus, the expression of the NOV6a gene could be used to distinguish bone marrow from the other samples in the panel. In addition, the expression of this gene could be used to distinguish adult heart tissue from fetal heart tissue.

The NOV6a gene is also expressed in many tissues with metabolic function, including the heart, fetal and adult liver, skeletal muscle and adrenal gland. The protein encoded by the NOV6a gene is a lipase homolog and may be involved in the dynamic mobilization of fat in these tissues. Therefore, administration of this gene product or an agonist designed to it could enhance lipolysis and may act as an effective therapy against obesity and lipodystrophy. Conversely, an antagonist of this gene product may be useful in the treatment of conditions involving excessive depletion of fat reserves, such as cachexia.

Panel 1.3D Summary: Ag1456/Ag2132/Ag2444 Three out of four experiments using different probe and primer sets show expression of the NOV6a gene in bone marrow (CTs=33-34) and the lung (CT=32.4). The high expression in bone marrow is consistent with its expression seen in Panel 1.2. Thus, the expression of the NOV6a gene could be used to distinguish samples derived from bone marrow and lung from other tissues on this panel. Furthermore, expression of the NOV6a gene could be used to distinguish between adult and fetal lung tissue. Ag2059/Ag2446 Expression of the gene is low/undetectable (Ct values>35) in all samples in Panel 1.3D (data not shown).

Panel 2D Summary: Ag1456 Three experiments with the same probe and primer produce results that are in excellent agreement, with highest expression of the NOV6a gene in normal lung tissue adjacent to a tumor (CTs=30-31). In addition, the NOV6a gene appears to be overexpressed in three pairs of normal lung tissue when compared to corresponding cancerous tissue. In addition, four of nine kidney cancers show overexpression of this gene when compared to their respective normal adjacent tissue. Thus, the expression of the NOV6a gene could be used to distinguish normal lung tissue from malignant lung tissue as well as malignant kidney from normal kidney. Moreover, therapeutic modulation of the expression of the CG50159-01 gene or its gene product, through the use of small molecule drugs, antibodies or protein therapeutics may be effective in the treatment of kidney cancer or lung cancer.

Panel 4D Summary: Ag1456/Ag1899/Ag2059/Ag2132 Multiple experiments with different probe and primer sets show highest expression of the NOV6a gene in resting monocytes (CTs=29-32). The gene appears to be downregulated in these cells following LPS treatment (CTs=32-34) and is not expressed at detectable levels in macrophages. The protein encoded by the NOV6a gene is homologous to acidic lipases and may play a role in lipid metabolism, differentiation, and activities such as phagocytosis, of these cells. Therefore, therapeutic modulation of the expression or function of the NOV6a gene or its protein product, through the use protein therapeutics, could regulate monocyte function and/or differentiation.

Conversely, modulation of the expression or activity of the putative protein encoded by this transcript by antibodies or small molecules can reduce or prevent the inflammatory symptoms associated with accumulation of monocytes observed in diseases such as asthma, allergies, inflammatory bowel disease, lupus erythematosus, or rheumatoid arthritis. Please note that results from two other experiments, designated 144575331 and 164391568 were not included. Bad amp plots indicate that there were experimental difficulties with these experiments.

G. NOV7: Tryptase 4

Expression of the NOV7 gene (CG56140-01) was assessed using the primer-probe sets Ag2886 and Ag2887, described in Tables 57 and 58. Results of the RTQ-PCR runs are shown in Tables 59 and 60.

TABLE 57
Probe Name Ag2886

| Primers | Sequences | Length | Start Position | SEQ ID NO: |
|---|---|---|---|---|
| Forward | 5′-cccacctgaactcctaaattgt-3′ | 22 | 1071 | 222 |
| Probe | TET-5′-ffttgttgcgaacagcagcaccct-3′-TAMRA | 24 | 1102 | 223 |
| Reverse | 5′-atctttccgatggaaataacca-3′ | 22 | 1127 | 224 |

TABLE 58
Probe Name Ag2887

| Primers | Sequences | Length | Start Position | SEQ ID NO: |
|---|---|---|---|---|
| Forward | 5′-aatcacacaaatgccagatgtt-3′ | 22 | 1255 | 225 |
| Probe | TET-5′-cactccaatggttgacctaaaaccagg-3′-TAMRA | 27 | 1294 | 226 |
| Reverse | 5′-agataaactaccgcacccatgt-3′ | 22 | 1321 | 227 |

TABLE 59
Panel 1.3D

| | Rel. Exp. (%) | | Rel. Exp. (%) |
|---|---|---|---|
| | Ag2886, Run | | Ag2886, Run |
| Tissue Name | 160838439 | Tissue Name | 160838439 |
| Liver adenocarcinoma | 0.0 | Kidney (fetal) | 24.7 |
| Pancreas | 0.0 | Renal ca. 786-0 | 0.0 |
| Pancreatic ca. CAPAN2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) HepG2 | 0.0 |
| Brain (cerebellum) | 0.0 | Lung | 0.0 |
| Brain (hippocampus) | 31.2 | Lung (fetal) | 0.0 |
| Brain (substantia nigra) | 15.0 | Lung ca. (small cell) LX-1 | 0.0 |
| Brain (thalamus) | 0.0 | Lung ca. (small cell) NCI-H69 | 0.0 |
| Cerebral Cortex | 0.0 | Lung ca. (s. cell var.) SHP-77 | 0.0 |
| Spinal cord | 0.0 | Lung ca. (large cell) NCI-H460 | 0.0 |
| glio/astro U87-MG | 0.0 | Lung ca. (non-sm. cell) A549 | 0.0 |
| glio/astro U-118-MG | 78.5 | Lung ca. (non-s. cell) NCI-H23 | 0.0 |
| astrocytoma SW1783 | 0.0 | Lung ca. (non-s. cell) HOP-62 | 0.0 |
| neuro*; met SK-N-AS | 9.4 | Lung ca. (non-s. cl) NCI-H522 | 0.0 |
| astrocytoma SF-539 | 0.0 | Lung ca. (squam.) SW 900 | 0.0 |
| astrocytoma SNB-75 | 0.0 | Lung ca. (squam.) NCI-H596 | 0.0 |
| glioma SNB-19 | 0.0 | Mammary gland | 0.0 |
| glioma U251 | 0.0 | Breast ca.* (pl. ef) MCF-7 | 0.0 |
| glioma SF-295 | 0.0 | Breast ca.* (pl. ef) MDA-MB- | 0.0 |
| | | 231 | |
| Heart (fetal) | 0.0 | Breast ca.* (pl. ef) T47D | 0.0 |
| Heart | 0.0 | Breast ca. BT-549 | 19.5 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 23.3 |
| Colorectal | 100.0 | Ovarian ca. IGROV-1 | 0.0 |
| Stomach | 0.0 | Ovarian ca.* (ascites) SK-OV-3 | 0.0 |
| Small intestine | 0.0 | Uterus | 0.0 |
| Colon ca. SW480 | 0.0 | Placenta | 0.0 |
| Colon ca.* SW620 (SW480 met) | 0.0 | Prostate | 0.0 |
| Colon ca. HT29 | 0.0 | Prostate ca.* (bone met) PC-3 | 0.0 |
| Colon ca. HCT-116 | 0.0 | Testis | 45.4 |
| Colon ca. CaCo-2 | 0.0 | Melanoma Hs688(A).T | 0.0 |
| Colon ca. tissue (ODO3866) | 0.0 | Melanoma* (met) Hs688(B).T | 0.0 |
| Colon ca. HCC-2998 | 13.6 | Melanoma UACC-62 | 0.0 |
| Gastric ca.* (liver met) NCI-N87 | 0.0 | Melanoma M14 | 0.0 |
| Bladder | 8.0 | Melanoma LOX IMVI | 0.0 |
| Trachea | 0.0 | Melanoma* (met) SK-MEL-5 | 0.0 |
| Kidney | 0.0 | Adipose | 0.0 |

TABLE 60
Panel 4D

| | Rel. Exp. (%) | Rel. Exp. (%) | | Rel. Exp. (%) | Rel. Exp. (%) |
|---|---|---|---|---|---|
| | Ag2886, Run | Ag2887, Run | | Ag2886, Run | Ag2887, Run |
| Tissue Name | 164031519 | 159843272 | Tissue Name | 164031519 | 159843272 |
| Secondary Th1 act | 0.0 | 0.0 | HUVEC IL-1beta | 0.0 | 0.0 |
| Secondary Th2 act | 10.0 | 0.0 | HUVEC IFN | 0.0 | 0.0 |
| | | | gamma | | |
| Secondary Tr1 act | 0.0 | 0.0 | HUVEC TNF | 0.0 | 0.0 |
| | | | alpha + IFN gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 0.0 | 0.0 | Lung Microvascular | 0.0 | 0.0 |
| | | | EC none | | |
| Primary Th1 act | 19.3 | 0.0 | Lung Microvascular | 0.0 | 0.0 |
| | | | EC TNFalpha + IL- | | |
| | | | 1beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 0.0 |
| | | | Dermal EC | | |
| | | | TNFalpha + IL- | | |
| | | | 1beta | | |
| Primary Th1 rest | 0.0 | 0.0 | Bronchial | 0.0 | 0.0 |
| | | | epithelium | | |
| | | | TNFalpha + IL1beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium | | |
| | | | TNFalpha + IL- | | |
| | | | 1beta | | |
| CD45RA CD4 | 0.0 | 0.0 | Coronery artery | 0.0 | 0.0 |
| lymphocyte act | | | SMC rest | | |
| CD45RO CD4 | 12.6 | 0.0 | Coronery artery | 0.0 | 0.0 |
| lymphocyte act | | | SMC TNFalpha + | | |
| | | | IL-1beta | | |
| CD8 lymphocyte act | 0.0 | 0.0 | Astrocytes rest | 0.0 | 0.0 |
| Secondary CD8 | 0.0 | 0.0 | Astrocytes | 0.0 | 0.0 |
| lymphocyte rest | | | TNFalpha + IL- | | |
| | | | 1beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 0.0 | 0.0 |
| none | | | PMA/ionomycin | | |
| 2ry | 0.0 | 0.0 | CCD1106 | 0.0 | 0.0 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.0 |
| | | | (Keratinocytes) | | |
| | | | TNFalpha + IL- | | |
| | | | 1beta | | |
| LAK cells IL-2 | 0.0 | 0.0 | Liver cirrhosis | 48.6 | 62.0 |
| LAK cells IL-2 + IL- | 0.0 | 0.0 | Lupus kidney | 0.0 | 0.0 |
| 12 | | | | | |
| LAK cells IL-2 + IFN | 0.0 | 0.0 | NCI-H292 none | 0.0 | 0.0 |
| gamma | | | | | |
| LAK cells IL-2 + IL- | 0.0 | 15.0 | NCI-H292 IL-4 | 0.0 | 0.0 |
| 18 | | | | | |
| LAK cells | 29.5 | 0.0 | NCI-H292 IL-9 | 0.0 | 0.0 |
| PMA/ionomycin | | | | | |
| NK Cells IL-2 rest | 0.0 | 0.0 | NCI-H292 IL-13 | 0.0 | 0.0 |
| Two Way MLR 3 | 0.0 | 0.0 | NCI-H292 IFN | 0.0 | 0.0 |
| day | | | gamma | | |
| Two Way MLR 5 | 0.0 | 0.0 | HPAEC none | 0.0 | 0.0 |
| day | | | | | |
| Two Way MLR 7 | 0.0 | 0.0 | HPAEC TNF | 0.0 | 0.0 |
| day | | | alpha + IL-1 beta | | |
| PBMC rest | 0.0 | 0.0 | Lung fibroblast | 0.0 | 0.0 |
| | | | none | | |
| PBMC PWM | 0.0 | 0.0 | Lung fibroblast | 0.0 | 0.0 |
| | | | TNF alpha + IL-1 | | |
| | | | beta | | |
| PBMC PHA-L | 0.0 | 0.0 | Lung fibroblast IL-4 | 0.0 | 0.0 |
| Ramos (B cell) none | 0.0 | 0.0 | Lung fibroblast IL-9 | 0.0 | 0.0 |
| Ramos (B cell) | 4.6 | 0.0 | Lung fibroblast IL- | 0.0 | 0.0 |
| ionomycin | | | 13 | | |
| B lymphocytes | 32.1 | 10.7 | Lung fibroblast IFN | 0.0 | 0.0 |
| PWM | | | gamma | | |
| B lymphocytes | 15.7 | 0.0 | Dermal fibroblast | 0.0 | 0.0 |
| CD40L and IL-4 | | | CCD1070 rest | | |
| EOL-1 dbcAMP | 0.0 | 0.0 | Dermal fibroblast | 0.0 | 0.0 |
| | | | CCD1070 TNF | | |
| | | | alpha | | |
| EOL-1 dbcAMP | 0.0 | 0.0 | Dermal fibroblast | 0.0 | 0.0 |
| PMA/ionomycin | | | CCD1070 IL-1 beta | | |
| Dendritic cells none | 0.0 | 0.0 | Dermal fibroblast | 0.0 | 0.0 |
| | | | IFN gamma | | |
| Dendritic cells LPS | 36.1 | 0.0 | Dermal fibroblast | 0.0 | 0.0 |
| | | | IL-4 | | |
| Dendritic cells anti- | 0.0 | 0.0 | IBD Colitis 2 | 0.0 | 12.7 |
| CD40 | | | | | |
| Monocytes rest | 0.0 | 0.0 | IBD Crohn's | 0.0 | 0.0 |
| Monocytes LPS | 0.0 | 0.0 | Colon | 100.0 | 100.0 |
| Macrophages rest | 0.0 | 0.0 | Lung | 8.0 | 14.7 |
| Macrophages LPS | 0.0 | 0.0 | Thymus | 16.2 | 0.0 |
| HUVEC none | 0.0 | 0.0 | Kidney | 0.0 | 0.0 |
| HUVEC starved | 0.0 | 0.0 | | | |

CNS_neurodegeneration_v1.0 Summary: Ag2886/Ag2887 Expression of the NOV7 gene is low/undetectable (CTs>35) in all samples on this panel. (Data not shown.)

Panel 1.3D Summary: Ag2886 Expression of the NOV7gene is restricted to normal colorectal tissue (CT=34.8). Thus, expression of this gene could be used to differentiate between this sample and other samples on this panel and between colorectal tissue and other normal and malignant tissue. Two other experiments with the probe and primer set Ag2877 showed low/undetectable (CTs>35) level of expression in all the samples on this panel. (Data not shown.)

Panel 2D Summary: Ag2886/Ag2887 Expression of the NOV7 gene is low/undetectable (CTs>35) in all samples on this panel. (Data not shown.)

Panel 3D Summary: Ag2887 Expression of the NOV7 gene is low/undetectable (CTs>35) in all samples on this panel. (Data not shown.)

Panel 4D Summary: Ag2886/Ag2887 Expression of the NOV7 gene is restricted to normal colon tissue (CTs=34.5). Furthermore, expression of this gene is undetectable in samples derived from patients with inflammatory bowel disease. Therefore, expression of the NOV7 transcript could be used to used to differentiate between normal and diseased colon. Furthermore, the highly specific expression of the NOV7 gene in colorectal tissue in this panel and panel 1.3D suggest that therapeutic modulation of the activity of the protein encoded by this gene may be useful in the treatment of inflammatory bowel disease.

H. NOV9: MITSUGUMIN 29

Expression of the NOV9 gene (CG56207-01) was assessed using the primer-probe set Ag2284, described in Table 61. Results of the RTQ-PCR runs are shown in Tables 62 and 63.

TABLE 61
Probe Name Ag2284

| | | | | SEQ ID |
|---|---|---|---|---|
| Primers | Sequences | Length | Start Position | NO: |
| Forward | 5′-tagttatctacctgcgcttcca-3′ | 22 | 386 | 228 |
| Probe | TET-5′-tctacacagagaacaaacgcttcccg-3′-TAMRA | 26 | 413 | 229 |
| Reverse | 5′-gaaggtgaaggagacagtcaca-3′ | 22 | 453 | 230 |

TABLE 62
Panel 1.3D

| | Rel. Exp. (%) | | Rel. Exp. (%) |
|---|---|---|---|
| | Ag2284, Run | | Ag2284, Run |
| Tissue Name | 167985231 | Tissue Name | 1167985231 |
| Liver adenocarcinoma | 0.2 | Kidney (fetal) | 1.6 |
| Pancreas | 0.3 | Renal ca. 786-0 | 0.0 |
| Pancreatic ca. CAPAN2 | 0.0 | Renal ca. A498 | 0.0 |
| Adrenal gland | 0.5 | Renal ca. RXF 393 | 0.0 |
| Thyroid | 1.2 | Renal ca. ACHN | 0.0 |
| Salivary gland | 0.4 | Renal ca. UO-31 | 0.0 |
| Pituitary gland | 0.1 | Renal ca. TK-10 | 0.0 |
| Brain (fetal) | 0.0 | Liver | 0.4 |
| Brain (whole) | 0.2 | Liver (fetal) | 0.1 |
| Brain (amygdala) | 0.2 | Liver ca. (hepatoblast) HepG2 | 0.1 |
| Brain (cerebellum) | 0.1 | Lung | 0.0 |
| Brain (hippocampus) | 0.1 | Lung (fetal) | 0.1 |
| Brain (substantia nigra) | 0.1 | Lung ca. (small cell) LX-1 | 0.0 |
| Brain (thalamus) | 0.1 | Lung ca. (small cell) NCI-H69 | 0.0 |
| Cerebral Cortex | 0.2 | Lung ca. (s. cell var.) SHP-77 | 0.0 |
| Spinal cord | 0.1 | Lung ca. (large cell) NCI-H466 | 0.0 |
| glio/astro U87-MG | 0.0 | Lung ca. (non-sm. cell) A549 | 0.0 |
| glio/astro U-118-MG | 0.2 | Lung ca. (non-s. cell) NCI-H23 | 0.5 |
| astrocytoma SW1783 | 0.1 | Lung ca. (non-s. cell) HOP-62 | 0.0 |
| neuro*; met SK-N-AS | 0.0 | Lung ca. (non-s. cl) NCI-H522 | 8.1 |
| astrocytoma SF-539 | 0.0 | Lung ca. (squam.) SW 900 | 0.2 |
| astrocytoma SNB-75 | 0.1 | Lung ca. (squam.) NCI-H596 | 0.0 |
| glioma SNB-19 | 0.0 | Mammary gland | 0.2 |
| glioma U251 | 0.1 | Breast ca.* (pl. ef) MCF-7 | 0.0 |
| glioma SF-295 | 0.0 | Breast ca.* (pl. ef) MDA-MB- | 0.0 |
| | | 231 | |
| Heart (fetal) | 1.8 | Breast ca.* (pl. ef) T47D | 0.1 |
| Heart | 2.3 | Breast ca. BT-549 | 0.2 |
| Skeletal muscle (fetal) | 100.0 | Breast ca. MDA-N | 0.0 |
| Skeletal muscle | 88.3 | Ovary | 0.8 |
| Bone marrow | 0.2 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.1 |
| Lymph node | 0.1 | Ovarian ca. OVCAR-8 | 0.1 |
| Colorectal | 0.0 | Ovarian ca. IGROV-1 | 0.0 |
| Stomach | 0.2 | Ovarian ca.* (ascites) SK-OV-3 | 0.1 |
| Small intestine | 0.2 | Uterus | 1.0 |
| Colon ca. SW480 | 0.1 | Placenta | 0.2 |
| Colon ca.* SW620 (SW480 met) | 0.0 | Prostate | 0.2 |
| Colon ca. HT29 | 0.0 | Prostate ca.* (bone met) PC-3 | 0.0 |
| Colon ca. HCT-116 | 0.2 | Testis | 1.1 |
| Colon ca. CaCo-2 | 0.1 | Melanoma Hs688(A).T | 0.0 |
| Colon ca. tissue (ODO3866) | 0.1 | Melanoma* (met) Hs688(B).T | 0.0 |
| Colon ca. HCC-2998 | 0.0 | Melanoma UACC-62 | 0.1 |
| Gastric ca.* (liver met) NCI-N87 | 0.1 | Melanoma M14 | 0.0 |
| Bladder | 0.2 | Melanoma LOX IMVI | 0.0 |
| Trachea | 0.1 | Melanoma* (met) SK-MEL-5 | 0.0 |
| Kidney | 2.8 | Adipose | 0.7 |

TABLE 63
Panel 4.1D

| | Rel. Exp. (%) | | Rel. Exp. (%) |
|---|---|---|---|
| | Ag2284, Run | | Ag2284, Run |
| Tissue Name | 170069125 | Tissue Name | 170069125 |
| Secondary Th1 act | 0.0 | HUVEC IL-1beta | 0.0 |
| Secondary Th2 act | 1.0 | HUVEC IFN gamma | 0.0 |
| Secondary Tr1 act | 0.0 | HUVEC TNF alpha + IFN gamma | 0.0 |
| Secondary Th1 rest | 0.7 | HUVEC TNF alpha + IL4 | 0.0 |
| Secondary Th2 rest | 0.5 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + | 0.0 |
| | | IL-1beta | |
| Primary Th2 act | 0.7 | Microvascular Dermal EC none | 0.0 |
| Primary Tr1 act | 0.0 | Microsvasular Dermal EC | 0.0 |
| | | TNFalpha + IL-1beta | |
| Primary Th1 rest | 0.0 | Bronchial epithelium TNFalpha + | 1.0 |
| | | IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1beta | |
| CD45RA CD4 lymphocyte act | 7.5 | Coronery artery SMC rest | 0.0 |
| CD45RO CD4 lymphocyte act | 0.0 | Coronery artery SMC TNFalpha + | 0.0 |
| | | IL-1beta | |
| CD8 lymphocyte act | 0.0 | Astrocytes rest | 1.9 |
| Secondary CD8 lymphocyte | 0.0 | Astrocytes TNFalpha + IL-1beta | 3.2 |
| rest | | | |
| Secondary CD8 lymphocyte act | 0.0 | KU-812 (Basophil) rest | 0.0 |
| CD4 lymphocyte none | 0.0 | KU-8I2 (Basophil) PMA/ionomycin | 0.9 |
| 2ry Th1/Th2/Tr1_anti-CD95 | 1.2 | CCD1106 (Keratinocytes) none | 0.0 |
| CH11 | | | |
| LAK cells rest | 0.8 | CCD1106 (Keratinocytes) | 0.0 |
| | | TNFalpha + IL-1beta | |
| LAK cells IL-2 | 0.0 | Liver cirrhosis | 2.2 |
| LAK cells IL-2 + IL-12 | 0.4 | NCI-H292 none | 0.8 |
| LAK cells IL-2 + IFN gamma | 0.0 | NCI-H292 IL-4 | 0.0 |
| LAK cells IL-2 + IL-18 | 0.0 | NCI-H292 IL-9 | 0.0 |
| LAK cells PMA/ionomycin | 1.5 | NCI-H292 IL-13 | 0.0 |
| NK Cells IL-2 rest | 1.3 | NCI-H292 IFN gamma | 0.0 |
| Two Way MLR 3 day | 1.3 | HPAEC none | 0.0 |
| Two Way MLR 5 day | 1.8 | HPAEC TNF alpha + IL-1 beta | 0.0 |
| Two Way MLR 7 day | 0.0 | Lung fibroblast none | 27.9 |
| PBMC rest | 0.0 | Lung fibroblast TNF alpha + IL-1 | 4.7 |
| | | beta | |
| PBMC PWM | 0.9 | Lung fibroblast IL-4 | 19.3 |
| PBMC PHA-L | 0.0 | Lung fibroblast IL-9 | 32.3 |
| Ramos (B cell) none | 0.0 | Lung fibroblast IL-13 | 11.4 |
| Ramos (B cell) ionomycin | 0.0 | Lung fibroblast IFN gamma | 9.9 |
| B lymphocytes PWM | 0.8 | Dermal fibroblast CCD1070 rest | 43.2 |
| B lymphocytes CD40L and IL-4 | 0.0 | Dermal fibroblast CCD1070 TNF | 31.0 |
| | | alpha | |
| EOL-1 dbcAMP | 0.0 | Dermal fibroblast CCD1070 IL-1 | 7.4 |
| | | beta | |
| EOL-1 dbcAMP | 0.0 | Dermal fibroblast IFN gamma | 5.8 |
| PMA/ionomycin | | | |
| Dendritic cells none | 0.0 | Dermal fibroblast IL-4 | 38.4 |
| Dendritic cells LPS | 0.5 | Dermal Fibroblasts rest | 24.7 |
| Dendritic cells anti-CD40 | 0.9 | Neutrophils TNFa + LPS | 0.0 |
| Monocytes rest | 0.0 | Neutrophils rest | 0.0 |
| Monocytes LPS | 2.4 | Colon | 1.0 |
| Macrophages rest | 8.9 | Lung | 7.3 |
| Macrophages LPS | 0.0 | Thymus | 3.1 |
| HUVEC none | 0.0 | Kidney | 100.0 |
| HUVEC starved | 0.0 | | |

Panel 1.3D Summary: Ag2284 The NOV9 gene, a mitsugumin 29 homolog, is most highly expressed in fetal skeletal muscle (CT=26.3) and adult skeletal muscle (CT=26.4). Much lower but significant expression is also detected in adipose, testis, uterus, ovary, kidney, heart, thyroid and adrenal gland (CTs=31-33). Thus, expression of the NOV9 gene could be used to distinguish skeletal muscle from other tissues. Nishi M. et all have shown that mitsugumin is essential for proper function of muscle. Therefore, therapeutic modulation of the NOV9 gene or gene product, through replacement therapy, could be used as a regenerative therapy for muscle disease.

References:

1. Nishi M., Komazaki S., Kurebayashi N., Ogawa Y., Noda T., Tino M., Takeshima H. (1999) Abnormal features in skeletal muscle from mice lacking mitsugumin29. J. Cell Biol. 147:1473-1480.

Physiological roles of the members of the synaptophysin family, carrying four transmembrane segments and being basically distributed on intracellular membranes including synaptic vesicles, have not been established yet. Recently, mitsugumin29 (MG29) was identified as a novel member of the synaptophysin family from skeletal muscle. MG29 is expressed in the junctional membrane complex between the cell surface transverse (T) tubule and the sarcoplasmic reticulum (SR), called the triad junction, where the depolarization signal is converted to Ca(2+) release from the SR. In this study, we examined biological functions of MG29 by generating knockout mice. The MG29-deficient mice exhibited normal health and reproduction but were slightly reduced in body weight. Ultrastructural abnormalities of the membranes around the triad junction were detected in skeletal muscle from the mutant mice, i.e., swollen T tubules, irregular SR structures, and partial misformation of triad junctions. In the mutant muscle, apparently normal tetanus tension was observed, whereas twitch tension was significantly reduced. Moreover, the mutant muscle showed faster decrease of twitch tension under Ca(2+)-free conditions. The morphological and functional abnormalities of the mutant muscle seem to be related to each other and indicate that MG29 is essential for both refinement of the membrane structures and effective excitation-contraction coupling in the skeletal muscle triad junction. Our results further imply a role of MG29 as a synaptophysin family member in the accurate formation of junctional complexes between the cell surface and intracellular membranes.

PMID: 10613905

Panel 4.1D Summary: Ag2284 Significant expression of the NOV9 gene in this panel is seen mainly in kidney. Furthermore, the homologous mitsugumin29 gene is also expressed in the kidney and is thought to be involved in secretory activities and perhaps in specialized endoplasmic reticulum systems (Ref. 1). Therefore, therapeutic drugs designed against the NOV9 gene product may be important for regulating the function of the kidney.

References:

1. Shimuta M., Komazaki S., Nishi M., Iino M., Nakagawara K., Takeshima H. (1998) Structure and expression of mitsugumin29 gene. FEBS Lett. 431:263-267.

Recently mitsugumin29 unique to the triad junction in skeletal muscle was identified as a novel member of the synaptophysin family; the members of this family have four transmembrane segments and are distributed on intracellular vesicles. In this study, we isolated and analyzed mouse mitsugumin29 cDNA and genomic DNA containing the gene. The mitsugumin29 gene mapped to the mouse chromosome 3 F3-H2 is closely related to the synaptophysin gene in exon-intron organization, which indicates their intimate relationship in molecular evolution. RNA blot hybridization and immunoblot analysis revealed that mitsugumin29 is expressed abundantly in skeletal muscle and at lower levels in the kidney. Immunofluorescence microscopy demonstrated that mitsugumin29 exists specifically in cytoplasmic regions of the proximal and distal tubule cells in the kidney. The results obtained may suggest that mitsugumin29 is involved in the formation of specialized endoplasmic reticulum systems in skeletal muscle and renal tubule cells.

PMID: 9708916

I. NOV10: Micromolar Calcium Activated Neutral Protease 1 Like

Expression of the NOV10 gene (CG56127-01) was assessed using the primer-probe sets Ag2885 and Ag2882, described in Tables 64 and 65. Results of the RTQ-PCR runs are shown in Tables 66, 67, 68 and 69.

TABLE 64
Probe Name Ag2885

| | | | | SEQ ID |
|---|---|---|---|---|
| Primers | Sequences | Length | Start Postion | NO: |
| Forward | 5′-ttcagaaacactgtccaaagct-3′ | 22 | 1592 | 231 |
| Probe | TET-5′-caccatgacttaccatctgagccctg-3′-TAMRA | 26 | 1639 | 232 |
| Reverse | 5′-gtgtctgtgcaaccacaacata-3′ | 22 | 1670 | 233 |

TABLE 65
Probe Name Ag2882

| | | | | SEQ ID |
|---|---|---|---|---|
| Primers | Sequences | Length | Start Position | NO: |
| Forward | 5′-ttcagaaacactgtccaaagct-3′ | 22 | 1592 | 234 |
| Probe | TET-5′-caccatgacttaccatctgagccctg-3′-TAMRA | 26 | 1639 | 235 |
| Reverse | 5′-gtgtctgtgcaaccacaacata-3′ | 22 | 1670 | 236 |

TABLE 66
CNS_neurodegeneration_v1.0

| | Rel. Exp. (%) | | Rel. Exp. (%) |
|---|---|---|---|
| | Ag2885, Run | | Ag2885, Run |
| Tissue Name | 219923398 | Tissue Name | 219923398 |
| AD 1 Hippo | 6.8 | Control (Path) 3 Temporal Ctx | 3.0 |
| AD 2 Hippo | 28.7 | Control (Path) 4 Temporal Ctx | 16.2 |
| AD 3 Hippo | 11.0 | AD 1 Occipital Ctx | 6.9 |
| AD 4 Hippo | 8.2 | AD 2 Occipital Ctx (Missing) | 0.0 |
| AD 5 Hippo | 33.9 | AD 3 Occipital Ctx | 0.0 |
| AD 6 Hippo | 28.9 | AD 4 Occipital Ctx | 21.2 |
| Control 2 Hippo | 34.6 | AD 5 Occipital Ctx | 30.6 |
| Control 4 Hippo | 8.8 | AD 6 Occipital Ctx | 36.1 |
| Control (Path) 3 Hippo | 1.8 | Control 1 Occipital Ctx | 0.0 |
| AD 1 Temporal Ctx | 12.3 | Control 2 Occipital Ctx | 31.2 |
| AD 2 Temporal Ctx | 15.9 | Control 3 Occipital Ctx | 9.8 |
| AD 3 Temporal Ctx | 12.8 | Control 4 Occipital Ctx | 8.0 |
| AD 4 Temporal Ctx | 18.3 | Control (Path) 1 Occipital Ctx | 68.3 |
| AD 5 Inf Temporal Ctx | 100.0 | Control (Path) 2 Occipital Ctx | 4.2 |
| AD 5 Sup Temporal Ctx | 41.8 | Control (Path) 3 Occipital Ctx | 4.1 |
| AD 6 Inf Temporal Ctx | 79.0 | Control (Path) 4 Occipital Ctx | 17.3 |
| AD 6 Sup Temporal Ctx | 17.3 | Control 1 Parietal Ctx | 5.3 |
| Control 1 Temporal Ctx | 4.2 | Control 2 Parietal Ctx | 23.5 |
| Control 2 Temporal Ctx | 32.5 | Control 3 Parietal Ctx | 17.8 |
| Control 3 Temporal Ctx | 8.7 | Control (Path) 1 Parietal Ctx | 48.0 |
| Control 3 Temporal Ctx | 8.7 | Control (Path) 2 Parietal Ctx | 15.1 |
| Control (Path) 1 Temporal Ctx | 32.1 | Control (Path) 3 Parietal Ctx | 3.0 |
| Control (Path) 2 Temporal Ctx | 8.2 | Control (Path) 4 Parietal Ctx | 35.6 |

TABLE 67
Panel 1.3D

| | Rel. Exp. (%) | Rel. Exp. (%) | | Rel. Exp. (%) | Rel. Exp. (%) |
|---|---|---|---|---|---|
| | Ag2882, Run | Ag2885, Run | | Ag2882, Run | Ag2885, Run |
| Tissue Name | 167649456 | 167649462 | Tissue Name | 167649456 | 167649462 |
| Liver | 0.0 | 0.0 | Kidney (fetal) | 1.1 | 17.6 |
| adenocarcinoma | | | | | |
| Pancreas | 0.8 | 13.1 | Renal ca. 786-0 | 0.1 | 1.5 |
| Pancreatic ca. | 0.0 | 0.0 | Renal ca. A498 | 0.1 | 0.0 |
| CAPAN2 | | | | | |
| Adrenal gland | 0.0 | 0.0 | Renal ca. RXF | 0.0 | 2.6 |
| | | | 393 | | |
| Thyroid | 0.0 | 0.0 | Renal ca. | 0.0 | 0.0 |
| | | | ACHN | | |
| Salivary gland | 0.0 | 1.3 | Renal ca. UO- | 0.0 | 0.0 |
| | | | 31 | | |
| Pituitary gland | 0.0 | 3.1 | Renal ca. TK- | 0.0 | 0.0 |
| | | | 10 | | |
| Brain (fetal) | 0.0 | 0.0 | Liver | 0.0 | 0.0 |
| Brain (whole) | 1.1 | 10.0 | Liver (fetal) | 0.6 | 3.6 |
| Brain (amygdala) | 0.2 | 2.7 | Liver ca. | 0.0 | 0.0 |
| | | | (hepatoblast) | | |
| | | | HepG2 | | |
| Brain (cerebellum) | 0.7 | 1.7 | Lung | 1.4 | 46.7 |
| Brain | 0.6 | 7.2 | Lung (fetal) | 0.7 | 7.6 |
| (hippocampus) | | | | | |
| Brain (substantia | 0.9 | 9.3 | Lung ca. (small | 0.0 | 0.0 |
| nigra) | | | cell) LX-1 | | |
| Brain (thalamus) | 0.7 | 15.9 | Lung ca. (small | 0.0 | 0.0 |
| | | | cell) NCI-H69 | | |
| Cerebral Cortex | 0.0 | 2.6 | Lung ca. (s. cell | 0.0 | 0.0 |
| | | | var.) SHP-77 | | |
| Spinal cord | 0.2 | 5.5 | Lung ca. (large | 0.0 | 0.0 |
| | | | cell) NCI-H460 | | |
| glio/astro U87-MG | 0.0 | 0.0 | Lung ca. (non- | 0.3 | 1.9 |
| | | | sm. cell) A549 | | |
| glio/astro U118- | 0.0 | 0.0 | Lung ca. (non- | 0.0 | 0.0 |
| MG | | | s. cell)NCI-H23 | | |
| astrocytoma | 0.0 | 0.0 | Lung ca. (non- | 0.0 | 0.0 |
| SW1783 | | | s. cell) HOP-62 | | |
| neuro*; met SK-N- | 0.0 | 1.1 | Lung ca. (non- | 0.0 | 0.0 |
| AS | | | s. cl) NCI-H522 | | |
| astrocytoma SF- | 0.0 | 0.0 | Lung ca. | 2.2 | 51.8 |
| 539 | | | (squam.) SW | | |
| | | | 900 | | |
| astrocytoma SNB- | 0.6 | 15.7 | Lung ca. | 0.0 | 0.0 |
| 75 | | | (squam.) NCI- | | |
| | | | H596 | | |
| glioma SNB-19 | 0.1 | 0.0 | Mammary | 3.2 | 55.5 |
| | | | gland | | |
| glioma U251 | 0.0 | 0.0 | Breast ca.* | 3.0 | 43.8 |
| | | | (pl. ef) MCF-7 | | |
| glioma SF-295 | 0.0 | 0.0 | Breast ca.* | 0.0 | 0.0 |
| | | | (pl. ef) MDA- | | |
| | | | MB-231 | | |
| Heart (fetal) | 0.0 | 0.0 | Breast ca.* | 4.4 | 90.1 |
| | | | (pl. ef) T47D | | |
| Heart | 0.0 | 0.0 | Breast ca. BT- | 0.0 | 0.0 |
| | | | 549 | | |
| Skeletal muscle | 0.1 | 5.6 | Breast ca. | 0.0 | 0.0 |
| (fetal) | | | MDA-N | | |
| Skeletal muscle | 0.0 | 0.0 | Ovary | 0.0 | 0.0 |
| Bone marrow | 0.0 | 1.5 | Ovarian ca. | 0.6 | 8.8 |
| | | | OVCAR-3 | | |
| Thymus | 0.0 | 0.0 | Ovarian ca. | 5.6 | 98.6 |
| | | | OVCAR-4 | | |
| Spleen | 0.0 | 1.8 | Ovarian ca. | 5.7 | 100.0 |
| | | | OVCAR-5 | | |
| Lymph node | 0.0 | 0.0 | Ovarian ca. | 0.0 | 3.1 |
| | | | OVCAR-8 | | |
| Colorectal | 5.6 | 61.6 | Ovarian ca. | 1.0 | 24.8 |
| | | | IGROV-1 | | |
| Stomach | 4.9 | 94.0 | Ovarian ca.* | 2.0 | 57.4 |
| | | | (ascites) SK- | | |
| | | | OV-3 | | |
| Small intestine | 2.0 | 54.7 | Uterus | 0.0 | 0.0 |
| Colon ca. SW480 | 0.0 | 0.0 | Placenta | 0.0 | 0.0 |
| Colon ca.* | 0.1 | 2.1 | Prostate | 0.2 | 2.9 |
| SW620(SW480 | | | | | |
| met) | | | | | |
| Colon ca. HT29 | 0.7 | 15.4 | Prostate ca.* | 0.0 | 0.0 |
| | | | (bone met)PC-3 | | |
| Colon ca. HCT-116 | 0.0 | 0.0 | Testis | 0.0 | 0.0 |
| Colon ca. CaCo-2 | 0.8 | 7.4 | Melanoma | 0.0 | 0.0 |
| | | | Hs688(A).T | | |
| Colon ca. | 0.0 | 0.0 | Melanoma* | 0.0 | 0.0 |
| tissue | | | (met) | | |
| (ODO3866) | | | Hs688(B).T | | |
| Colon ca. HCC- | 0.0 | 0.0 | Melanoma | 0.0 | 0.0 |
| 2998 | | | UACC-62 | | |
| Gastric ca.* (liver | 0.3 | 6.2 | Melanoma M14 | 0.0 | 0.0 |
| met) NCI-N87 | | | | | |
| Bladder | 0.7 | 13.3 | Melanoma LOX | 0.0 | 0.0 |
| | | | IMVI | | |
| Trachea | 100.0 | 15.4 | Melanoma* | 0.0 | 0.0 |
| | | | (met) SK-MEL- | | |
| | | | 5 | | |
| Kidney | 1.8 | 60.3 | Adipose | 0.0 | 0.0 |

TABLE 68
Panel 2.2

| | Rel. Exp. (%) | Rel. Exp. (%) | | Rel. Exp. (%) | Rel. Exp. (%) |
|---|---|---|---|---|---|
| | Ag2882, Run | Ag2885, Run | | Ag2882, Run | Ag2885, Run |
| Tissue Name | 175119370 | 175119371 | Tissue Name | 175119370 | 175119371 |
| Normal Colon | 14.9 | 22.8 | Kidney Margin | 41.5 | 46.0 |
| | | | (OD04348) | | |
| Colon cancer | 0.0 | 0.0 | Kidney malignant | 10.6 | 11.2 |
| (OD06064) | | | cancer | | |
| | | | (OD06204B) | | |
| Colon Margin | 5.8 | 12.9 | Kidney normal | 2.9 | 0.0 |
| (OD06064) | | | adjacent tissue | | |
| | | | (OD06204E) | | |
| Colon cancer | 0.0 | 0.0 | Kidney Cancer | 0.0 | 0.0 |
| (OD06159) | | | (OD04450-01) | | |
| Colon Margin | 9.2 | 6.6 | Kidney Margin | 12.9 | 10.2 |
| (OD06159) | | | (OD04450-03) | | |
| Colon cancer | 0.0 | 0.0 | Kidney Cancer | 0.0 | 0.0 |
| (OD06297-04) | | | 8120613 | | |
| Colon Margin | 39.5 | 38.4 | Kidney Margin | 7.4 | 5.3 |
| (OD06297-015) | | | 8120614 | | |
| CC Gr. 2 ascend | 2.5 | 1.6 | Kidney Cancer | 0.0 | 0.0 |
| colon (ODO3921) | | | 9010320 | | |
| CC Margin | 2.5 | 1.3 | Kidney Margin | 3.8 | 1.7 |
| (ODO3921) | | | 9010321 | | |
| Colon cancer | 0.0 | 2.1 | Kidney Cancer | 7.9 | 5.2 |
| metastasis | | | 8120607 | | |
| (OD06104) | | | | | |
| Lung Margin | 8.8 | 6.7 | Kidney Margin | 2.8 | 1.4 |
| (OD06104) | | | 8120608 | | |
| Colon mets to | 10.8 | 6.3 | Normal Uterus | 0.0 | 0.0 |
| lung (OD04451- | | | | | |
| 01) | | | | | |
| Lung Margin | 12.2 | 14.1 | Uterine Cancer | 47.6 | 50.0 |
| (OD04451-02) | | | 064011 | | |
| Normal Prostate | 0.0 | 2.1 | Normal Thyroid | 0.0 | 0.0 |
| Prostate Cancer | 0.0 | 0.0 | Thyroid Cancer | 0.0 | 0.0 |
| (OD04410) | | | 064010 | | |
| Prostate Margin | 0.0 | 0.0 | Thyroid Cancer | 0.0 | 0.0 |
| (OD04410) | | | A302152 | | |
| Normal Ovary | 0.0 | 0.0 | Thyroid Margin | 0.0 | 0.0 |
| | | | A302153 | | |
| Ovarian cancer | 0.0 | 2.7 | Normal Breast | 19.1 | 22.2 |
| (OD06283-03) | | | | | |
| Ovarian Margin | 0.0 | 0.0 | Breast Cancer | 0.0 | 6.1 |
| (OD06283-07) | | | (OD04566) | | |
| Ovarian Cancer | 10.1 | 5.4 | Breast Cancer | 71.2 | 72.2 |
| 064008 | | | 1024 | | |
| Ovarian cancer | 0.0 | 1.3 | Breast Cancer | 7.0 | 4.3 |
| (OD06145) | | | (OD04590-01) | | |
| Ovarian Margin | 0.0 | 0.0 | Breast Cancer | 7.4 | 3.4 |
| (OD06145) | | | Mets (OD04590- | | |
| | | | 03) | | |
| Ovarian cancer | 7.4 | 12.9 | Breast Cancer | 24.8 | 21.8 |
| (OD06455-03) | | | Metastasis | | |
| | | | (OD04655-05) | | |
| Ovarian Margin | 0.0 | 0.0 | Breast Cancer | 28.9 | 27.2 |
| (OD06455-07) | | | 064006 | | |
| Normal Lung | 2.4 | 5.6 | Breast Cancer | 18.6 | 20.7 |
| | | | 9100266 | | |
| Invasive poor diff. | 59.5 | 53.2 | Breast Margin | 11.2 | 10.2 |
| lung adeno | | | 9100265 | | |
| (ODO4945-01 | | | | | |
| Lung Margin | 12.1 | 4.2 | Breast Cancer | 10.2 | 21.8 |
| (ODO4945-03) | | | A209073 | | |
| Lung Malignant | 8.2 | 11.0 | Breast Margin | 7.2 | 10.1 |
| Cancer | | | A2090734 | | |
| (OD03126) | | | | | |
| Lung Margin | 2.0 | 6.7 | Breast cancer | 100.0 | 73.7 |
| (OD03126) | | | (OD06083) | | |
| Lung Cancer | 2.4 | 6.3 | Breast cancer | 54.3 | 49.0 |
| (OD05014A) | | | node metastasis | | |
| | | | (OD06083) | | |
| Lung Margin | 9.8 | 8.1 | Normal Liver | 0.0 | 0.0 |
| (OD05014B) | | | | | |
| Lung cancer | 2.6 | 1.7 | Liver Cancer | 0.0 | 0.0 |
| (OD06081) | | | 1026 | | |
| Lung Margin | 14.1 | 17.2 | Liver Cancer | 0.0 | 1.8 |
| (OD06081) | | | 1025 | | |
| Lung Cancer | 14.9 | 12.1 | Liver Cancer | 0.0 | 1.0 |
| (OD04237-01) | | | 6004-T | | |
| Lung Margin | 21.3 | 22.2 | Liver Tissue | 0.0 | 0.0 |
| (OD04237-02) | | | 6004-N | | |
| Ocular Melanoma | 0.0 | 0.0 | Liver Cancer | 8.9 | 1.7 |
| Metastasis | | | 6005-T | | |
| Ocular Melanoma | 0.0 | 1.8 | Liver Tissue | 0.0 | 0.0 |
| Margin (Liver) | | | 6005-N | | |
| Melanoma | 1.5 | 0.0 | Liver Cancer | 0.0 | 0.0 |
| Metastasis | | | 064003 | | |
| Melanoma | 21.3 | 8.3 | Normal Bladder | 3.5 | 0.0 |
| Margin (Lung) | | | | | |
| Normal Kidney | 9.7 | 5.6 | Bladder Cancer | 5.4 | 2.6 |
| | | | 1023 | | |
| Kidney Ca, | 13.1 | 11.9 | Bladder Cancer | 0.0 | 0.0 |
| Nuclear grade 2 | | | A302173 | | |
| (OD04338) | | | | | |
| Kidney Margin | 2.3 | 3.0 | Normal Stomach | 95.9 | 100.0 |
| (OD04338) | | | | | |
| Kidney Ca, | 24.0 | 24.7 | Gastric Cancer | 0.0 | 0.0 |
| Nuclear grade 1/2 | | | 9060397 | | |
| (OD04339) | | | | | |
| Kidney Margin | 16.6 | 12.5 | Stomach Margin | 11.9 | 2.6 |
| (OD04339) | | | 9060396 | | |
| Kidney Ca, Clear | 5.1 | 6.3 | Gastric Cancer | 0.0 | 1.2 |
| cell type | | | 9060395 | | |
| (OD04340) | | | | | |
| Kidney Margin | 15.2 | 11.6 | Stomach Margin | 24.1 | 21.8 |
| (OD04340) | | | 9060394 | | |
| Kidney Ca, | 0.0 | 0.0 | Gastric Cancer | 5.8 | 3.3 |
| Nuclear grade 3 | | | 064005 | | |
| (OD04348) | | | | | |

TABLE 69
Panel 4D

| | Rel. Exp. (%) | Rel. Exp. (%) | | Rel. Exp. (%) | Rel. Exp. (%) |
|---|---|---|---|---|---|
| | Ag2882, Run | Ag2885, Run | | Ag2882, Run | Ag2885, Run |
| Tissue Name | 164311038 | 164311039 | Tissue Name | 164311038 | 164311039 |
| Secondary Th1 act | 0.5 | 0.0 | HUVEC IL-1beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 0.0 | HUVEC TNF | 0.0 | 0.0 |
| | | | alpha + IFN gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 0.0 | 0.0 | Lung Microvascular | 0.0 | 0.0 |
| | | | EC none | | |
| Primary Th1 act | 0.0 | 0.0 | Lung Microvascular | 0.0 | 0.0 |
| | | | EC TNFalpha + IL- | | |
| | | | 1beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 0.0 |
| | | | Dermal EC | | |
| | | | TNFalpha + IL- | | |
| | | | 1beta | | |
| Primary Th1 rest | 0.0 | 0.0 | Bronchial | 0.0 | 0.0 |
| | | | epithelium | | |
| | | | TNFalpha + IL1beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.5 | 0.6 |
| | | | epithelium | | |
| | | | TNFalpha + IL- | | |
| | | | 1beta | | |
| CD45RA CD4 | 0.0 | 0.0 | Coronery artery | 0.0 | 0.0 |
| lymphocyte act | | | SMC rest | | |
| CD45RO CD4 | 0.0 | 0.0 | Coronery artery | 0.0 | 0.0 |
| lymphocyte act | | | SMC TNFalpha + | | |
| | | | IL-1beta | | |
| CD8 lymphocyte act | 0.0 | 0.0 | Astrocytes rest | 0.0 | 0.2 |
| Secondary CD8 | 0.4 | 0.0 | Astrocytes | 0.0 | 0.0 |
| lymphocyte rest | | | TNFalpha + IL- | | |
| | | | 1beta | | |
| Secondary CD8 | 0.0 | 0.0 | KU-812 (Basophil) | 0.0 | 0.4 |
| lymphocyte act | | | rest | | |
| CD4 lymphocyte | 0.0 | 0.0 | KU-812 (Basophil) | 0.0 | 0.0 |
| none | | | PMA/ionomycin | | |
| 2ry | 0.0 | 0.0 | CCD1106 | 0.0 | 0.0 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.0 |
| | | | (Keratinocytes) | | |
| | | | TNFalpha + IL- | | |
| | | | 1beta | | |
| LAK cells IL-2 | 0.0 | 0.0 | Liver cirrhosis | 1.3 | 0.7 |
| LAK cells IL-2 + IL- | 0.0 | 0.0 | Lupus kidney | 6.1 | 6.3 |
| 12 | | | | | |
| LAK cells IL-2 + IFN | 0.0 | 0.4 | NCI-H292 none | 0.0 | 0.5 |
| gamma | | | | | |
| LAK cells IL-2 + IL- | 0.0 | 0.0 | NCI-H292 IL-4 | 0.0 | 0.0 |
| 18 | | | | | |
| LAK cells | 0.0 | 0.0 | NCI-H292 IL-9 | 0.0 | 0.3 |
| PMA/ionomycin | | | | | |
| NK Cells IL-2 rest | 0.0 | 0.0 | NCI-H292 IL-13 | 0.5 | 0.0 |
| Two Way MLR 3 | 0.0 | 0.0 | NCI-H292 IFN | 0.0 | 0.3 |
| day | | | gamma | | |
| Two Way MLR 5 | 0.0 | 0.0 | HPAEC none | 0.0 | 0.0 |
| day | | | | | |
| Two Way MLR 7 | 0.0 | 0.0 | HPAEC TNF | 0.0 | 0.0 |
| day | | | alpha + IL-1 beta | | |
| PBMC rest | 0.0 | 0.0 | Lung fibroblast | 0.0 | 0.0 |
| | | | none | | |
| PBMC PWM | 0.0 | 0.0 | Lung fibroblast | 0.0 | 0.0 |
| | | | TNFalpha + IL-1 | | |
| | | | beta | | |
| PBMC PHA-L | 0.4 | 0.0 | Lung fibroblast IL-4 | 0.0 | 0.0 |
| Ramos (B cell) none | 0.0 | 0.0 | Lung fibroblast IL-9 | 0.0 | 0.0 |
| Ramos (B cell) | 0.0 | 0.0 | Lung fibroblast IL- | 0.0 | 0.0 |
| ionomycin | | | 13 | | |
| B lymphocytes | 0.5 | 0.2 | Lung fibroblast IFN | 0.0 | 0.0 |
| PWM | | | gamma | | |
| B lymphocytes | 0.0 | 0.0 | Dermal fibroblast | 0.0 | 0.0 |
| CD40L and IL-4 | | | CCD1070 rest | | |
| EOL-1 dbcAMP | 0.0 | 0.0 | Dermal fibroblast | 0.0 | 0.0 |
| | | | CCD1070 TNF | | |
| | | | alpha | | |
| EOL-1 dbcAMP | 0.0 | 0.0 | Dermal fibroblast | 0.0 | 0.0 |
| PMA/ionomycin | | | CCD1070 IL-1 beta | | |
| Dendritic cells none | 0.0 | 0.0 | Dermal fibroblast | 0.0 | 0.0 |
| | | | IFN gamma | | |
| Dendritic cells LPS | 0.0 | 0.0 | Dermal fibroblast | 0.0 | 0.2 |
| | | | IL-4 | | |
| Dendritic cells anti- | 0.0 | 0.0 | IBD Colitis 2 | 0.0 | 0.8 |
| CD40 | | | | | |
| Monocytes rest | 0.0 | 0.0 | IBD Crohn's | 17.7 | 21.6 |
| Monocytes LPS | 0.0 | 0.0 | Colon | 100.0 | 100.0 |
| Macrophages rest | 0.0 | 0.0 | Lung | 12.2 | 14.5 |
| Macrophages LPS | 0.0 | 0.0 | Thymus | 48.0 | 40.1 |
| HUVEC none | 0.0 | 0.0 | Kidney | 0.0 | 0.0 |
| HUVEC starved | 0.0 | 0.0 | | | |

CNS_neurodegeneration_v1.0 Summary: Ag2885 Expression of the NOV10 gene is widespread at low but significant levels in the adult central nervous system. From the results in this panel, this gene appears to be differentially expressed in Alzheimer's disease. Therefore, inhibition of this NOV10 protease homolog may be of benefit in the treatment of Alzheimer's disease.

Panel 1.3D Summary: Ag2882/2885 Two experiments with the same probe and primer set produce results that are in reasonable agreement. Highest expression of the NOV10 gene is seen in an ovarian cancer cell line (OVCAR-5) (CT=33) and the trachea (CT=30). In addition, there appears to be substantial expression in other ovarian cancer cell lines, breast cancer cell lines, colon tissue, small intestine tissue, stomach tissue, kidney tissue, lung tissue and mammary gland tissue. Thus, the expression of the NOV10 gene could be used to distinguish OVCAR-5 cells from other samples in the panel. Differential expression of calpain has been observed in a variety of cancers and Braun et. al (see reference below) have suggested that it probably plays a role in carcinogenesis and tumor progression. Therefore, therapeutic modulation of the NOV10 gene, through the use of small molecule drugs, antibodies or protein therapeutics might be beneficial in the treatment of ovarian cancer or breast cancer.

References:

Braun C, Engel M, Seifert M, Theisinger B, Seitz G, Zang K D, Welter C. Expression of calpain I messenger RNA in human renal cell carcinoma: correlation with lymph node metastasis and histological type. Int J Cancer Feb. 19, 1999;84(1):6-9

Calpain, also named CANP (for calcium-activated neutral protease), is an intracellular cytoplasmatic non-lysosomal cysteine endopeptidase that requires calcium ions for activity. Many substrates of the calpain isoenzymes, such as the transcription factors c-Fos and c-Jun, the tumor supressor protein p53, protein kinase C, pp60c-src and the adhesion molecule integrin, have been implicated in the pathogenesis of different human tumors, suggesting an important role of the calpains in malignant diseases. We now report differential expression of the calpain I gene (CL I) in a variety of tumors, extending our study to a larger series of renal cell carcinomas. Using Northern-blot analysis, we studied calpain I expression in 30 renal cell carcinomas as compared with matched healthy tissues. Tumor samples were classified according to their histological type: 21 clear cell carcinomas, 4 chromophobe carcinomas, 3 papillary carcinomas and 2 oncocytomas. In renal tumor samples, calpain I gene mRNA was expressed at highly variable levels, significantly depending on the different histological types. Moreover, there was a correlation of higher calpain I expression with increased malignancy: within the clear cell carcinoma subset, tumor samples with advanced nodal status (N1 and N2) showed a significantly higher calpain I expression than tumors without metastasis to regional lymph nodes. Our data suggest an important role of calpain isoenzymes in carcinogenesis and tumor progression.

PMID: 9988224

Panel 2.2 Summary: Ag2882/2885 Two experiments with the same probe and primer set produce results that are in reasonable agreement. Highest expression of the NOV10 gene is seen in samples derived from a breast cancer or normal stomach tissue (CTs=33). In addition, there appears to be substantial expression in other breast cancer samples, a uterine cancer sample and a lung cancer sample. Thus, the expression of the NOV10 gene could be used to distinguish these samples from other samples in the panel. The significant levels of expression in this calpain homolog are in concordance with published data showing differential expression of calpain in a variety of tumors and the suggestion that it plays a role in carcinogenesis and tumor progression. (Please see Panel 1.3D for references) Therefore, therapeutic modulation of the NOV10 gene, through the use of small molecule drugs, antibodies or protein therapeutics might be beneficial in the treatment of breast cancer, lung cancer or uterine cancer.

Panel 4D Summary: Ag2882/2885 Two experiments with the same probe and primer set produce results that are in reasonable agreement. Significant expression of the NOV10 gene is limited to normal colon, thymus and lung. The NOV10 transcript encodes for a calcium activated neutral protease like molecule. This family of molecules is implicated in cytoskeletal organization, cell proliferation, cell motility, and hemostasis. Therefore, the NOV10 gene product may play an important role in the normal homeostasis of these tissues. In addition, calpain I has been shown to inhibit the activation of NF-kappa B, and may be useful in the treatment of conditions associated with local or systemic inflammation. (See reference below) Thus, therapeutics designed with the protein encoded for by the NOV10 transcript using small molecules could be important for maintaining or restoring normal function to the lung, colon and thymus during inflammation.

References:

Ruetten H, Thiemermann C. Effect of calpain inhibitor I, an inhibitor of the proteolysis of I kappa B, on the circulatory failure and multiple organ dysfunction caused by endotoxin in the rat. Br J Pharmacol 1997 June;121(4):695-704

1. We compared the effects of calpain inhibitor I (inhibitor of the proteolysis of I kappa B and, hence, of the activation of nuclear factor kappa B (NF kappa B) and dexamethasone on (i) the circulatory failure, (ii) multiple organ dysfunction and (iii) induction of the inducible isoforms of nitric oxide (NO) synthase (iNOS) and cyclo-oxygenase (COX-2) in anaesthetized rats with endotoxic shock. 2. Injection of lipopolysaccharide (LPS, E. coli, 10 mg kg-1, i.v.) resulted in hypotension and a reduction of the pressor responses elicited by noradrenaline. This circulatory dysfunction was attenuated by pretreatment of LPS-rats with calpain inhibitor I (10 mg kg-1, i.v., 2 h before LPS) or dexamethasone (1 mg kg-1, i.v.). 3. Endotoxaemia also caused rises in the serum levels of (i) urea and creatinine (renal dysfunction), (ii) alanine aminotransferase (ALT), aspartate aminotransferase (AST) (hepatocellular injury), bilirubin and gamma-glutamyl transferase (gamma GT) (liver dysfunction), (iii) lipase (pancreatic injury) and (iv) lactate. Calpain inhibitor I and dexamethasone attenuated the liver injury, the pancreatic injury, the lactic acidosis as well as the hypoglycaemia caused by LPS. Dexamethasone, but not calpain inhibitor I, reduced the renal dysfunction caused by LPS. 4. Endotoxaemia for 6 h resulted in a substantial increase in iNOS and COX-2 protein and activity in lung and liver, which was attenuated in LPS-rats pretreated with calpain inhibitor I or dexamethasone. 5. Thus, calpain inhibitor I and dexamethasone attenuate (i) the circulatory failure, (ii) the multiple organ dysfunction (liver and pancreatic dysfunction/injury, lactic acidosis, hypoglycaemia), as well as (iii) the induction of iNOS and COX-2 protein and activity in rats with endotoxic shock. We propose that prevention of the activation of NF-kappa B in vivo may be useful in the therapy of circulatory shock or of disorders associated with local or systemic inflammation.

PMID: 9208136

J. NOV11: Novel P2×2C Receptor

Expression of the NOV11 gene (CG56179-01) was assessed using the primer-probe set Ag3491, described in Table 70. Results of the RTQ-PCR runs are shown in Tables 71 and 72.

TABLE 70
Probe Name Ag3491

| Primers | Sequences | Length | Start Position | SEQ ID NO: |
|---|---|---|---|---|
| Forward | 5′-agcctgattcccaccattatta-3′ | 22 | 937 | 237 |
| Probe | TET-5′-atctggccacagctctgacttccgt-3′-TAMRA | 25 | 959 | 238 |
| Reverse | 5′-accccagagagggttcctta-3′ | 20 | 992 | 239 |

TABLE 71
General_screening_panel_v1.4

| | Rel. Exp. (%) | | Rel. Exp. (%) |
|---|---|---|---|
| | Ag3491, Run | | Ag3491, Run |
| Tissue Name | 213390613 | Tissue Name | 213390613 |
| Adipose | 0.9 | Renal ca. TK-10 | 1.8 |
| Melanoma* Hs688(A).T | 0.0 | Bladder | 0.0 |
| Melanoma* Hs688(B).T | 0.0 | Gastric ca. (liver met.) NCI-N87 | 0.8 |
| Melanoma* M14 | 0.5 | Gastric ca. KATO III | 0.5 |
| Melanoma* LOXIMVI | 0.0 | Colon ca. SW-948 | 0.0 |
| Melanoma* SK-MEL-5 | 0.0 | Colon ca. SW480 | 1.5 |
| Squamous cell carcinoma SCC-4 | 0.0 | Colon ca.* (SW480 met) SW620 | 0.0 |
| Testis Pool | 33.2 | Colon ca. HT29 | 1.4 |
| Prostate ca.* (bone met) PC-3 | 0.0 | Colon ca. HCT-116 | 2.0 |
| Prostate Pool | 12.6 | Colon ca. CaCo-2 | 3.1 |
| Placenta | 1.5 | Colon cancer tissue | 1.5 |
| Uterus Pool | 0.0 | Colon ca. SW1116 | 0.0 |
| Ovarian ca. OVCAR-3 | 0.0 | Colon ca. Colo-205 | 3.2 |
| Ovarian ca. SK-OV-3 | 5.5 | Colon ca. SW-48 | 4.0 |
| Ovarian ca. OVCAR-4 | 1.7 | Colon Pool | 0.8 |
| Ovarian ca. OVCAR-5 | 2.6 | Small Intestine Pool | 2.2 |
| Ovarian ca. IGROV-1 | 3.1 | Stomach Pool | 0.0 |
| Ovarian ca. OVCAR-8 | 0.0 | Bone Marrow Pool | 1.3 |
| Ovary | 0.0 | Fetal Heart | 0.0 |
| Breast ca. MCF-7 | 100.0 | Heart Pool | 2.0 |
| Breast ca. MDA-MB-231 | 3.6 | Lymph Node Pool | 1.1 |
| Breast ca. BT 549 | 0.7 | Fetal Skeletal Muscle | 0.0 |
| Breast ca. T47D | 29.9 | Skeletal Muscle Pool | 1.0 |
| Breast ca. MDA-N | 0.4 | Spleen Pool | 6.7 |
| Breast Pool | 0.0 | Thymus Pool | 3.6 |
| Trachea | 36.9 | CNS cancer (glio/astro) U87-MG | 1.3 |
| Lung | 1.8 | CNS cancer (glio/astro) U-118-MG | 2.2 |
| Fetal Lung | 67.8 | CNS cancer (neuro; met) SK-N-AS | 1.7 |
| Lung ca. NCI-N417 | 0.7 | CNS cancer (astro) SF-539 | 0.0 |
| Lung ca. LX-1 | 2.1 | CNS cancer (astro) SNB-75 | 0.8 |
| Lung ca. NCI-H146 | 5.4 | CNS cancer (glio) SNB-19 | 2.6 |
| Lung ca. SHP-77 | 26.4 | CNS cancer (glio) SF-295 | 0.5 |
| Lung ca. A549 | 2.0 | Brain (Amygdala) Pool | 8.1 |
| Lung ca. NCI-H526 | 7.1 | Brain (cerebellum) | 0.0 |
| Lung ca. NCI-H23 | 15.7 | Brain (fetal) | 1.5 |
| Lung ca. NCI-H460 | 3.3 | Brain (Hippocampus) Pool | 9.2 |
| Lung ca. HOP-62 | 1.0 | Cerebral Cortex Pool | 4.9 |
| Lung ca. NCI-H522 | 1.5 | Brain (Substantia nigra) Pool | 1.4 |
| Liver | 0.0 | Brain (Thalamus) Pool | 17.1 |
| Fetal Liver | 0.0 | Brain (whole) | 6.0 |
| Liver ca. HepG2 | 0.0 | Spinal Cord Pool | 0.0 |
| Kidney Pool | 4.6 | Adrenal Gland | 0.6 |
| Fetal Kidney | 12.0 | Pituitary gland Pool | 0.0 |
| Renal ca. 786-0 | 1.1 | Salivary Gland | 0.0 |
| Renal ca. A498 | 0.0 | Thyroid (female) | 0.0 |
| Renal ca. ACHN | 1.7 | Pancreatic ca. CAPAN2 | 1.8 |
| Renal ca. UO-31 | 0.0 | Pancreas Pool | 8.4 |

TABLE 72
Panel 5 Islet

| | Rel. | | Rel. |
|---|---|---|---|
| | Exp. (%) | | Exp. (%) |
| | Ag3491, | | Ag3491, |
| | Run | | Run |
| Tissue Name | 242385402 | Tissue Name | 242385402 |
| 97457_Patient-02go_adipose | 0.0 | 94709_Donor 2 AM - A_adipose | 0.0 |
| 97476_Patient-07sk_skeletal | 0.0 | 94710_Donor 2 AM - B_adipose | 0.0 |
| muscle | | | |
| 97477_Patient-07ut_uterus | 0.0 | 94711_Donor 2 AM - C_adipose | 0.0 |
| 97478_Patient-07pl_placenta | 0.0 | 94712_Donor 2 AD - A_adipose | 0.0 |
| 99167_Bayer Patient 1 | 100.0 | 94713_Donor 2 AD - B_adipose | 0.0 |
| 97482_Patient-08ut_uterus | 0.0 | 94714_Donor 2 AD - C_adipose | 0.0 |
| 97483_Patient-08pl_placenta | 24.1 | 94742_Donor 3 U - A_Mesenchymal Stem | 0.0 |
| | | Cells | |
| 97486_Patient-09sk_skeletal | 0.0 | 94743_Donor 3 U - B_Mesenchymal Stem | 0.0 |
| muscle | | Cells | |
| 97487_Patient-09ut_uterus | 0.0 | 94730_Donor 3 AM - A_adipose | 0.0 |
| 97488_Patient-09pl_placenta | 19.9 | 94731_Donor 3 AM - B_adipose | 0.0 |
| 97492_Patient-10ut_uterus | 0.0 | 94732_Donor 3 AM - C_adipose | 0.0 |
| 97493_Patient-10pl_placenta | 0.0 | 94733_Donor 3 AD - A_adipose | 0.0 |
| 97495_Patient-11go_adipose | 0.0 | 94734_Donor 3 AD - B_adipose | 0.0 |
| 97496_Patient-11sk_skeletal | 0.0 | 94735_Donor 3 AD - C_adipose | 0.0 |
| muscle | | | |
| 97497_Patient-11ut_uterus | 0.0 | 77138_Liver_HepG2untreated | 0.0 |
| 97498_Patient-11pl_placenta | 15.3 | 73556_Heart_Cardiac stromal cells | 0.0 |
| | | (primary) | |
| 97500_Patient-12go_adipse | 0.0 | 81735_Small Intestine | 2.9 |
| 97501_Patient-12sk_skeletal | 0.0 | 72409_Kidney_Proximal Convoluted | 0.0 |
| muscle | | Tubule | |
| 97502_Patient-12ut_uterus | 0.0 | 82685_Small intestine_Duodenum | 0.0 |
| 97503_Patient-12pl_placenta | 0.0 | 90650_Adrenal_Adrenocortical adenoma | 0.0 |
| 94721_Donor 2 U - | 0.0 | 72410_Kidney_HRCE | 0.0 |
| A_Mesenchymal Stem Cells | | | |
| 94722_Donor 2 U - | 0.0 | 72411_Kidney_HRE | 0.0 |
| B_Mesenchymal Stem Cells | | | |
| 94723_Donor 2 U - | 0.0 | 73139_Uterus_Uterine smooth muscle | 0.0 |
| C_Mesenchymal Stem Cells | | cells | |

General_screening_panel-v1.4 Summary: Ag3491 The expression of the NOV11 gene appears to be highest in a sample derived from a breast cancer cell line (MCF-7)(CT=29.8). Thus, the expression of the NOV11 gene could be used to distinguish MCF-7 cells from the other samples in the panel. Moreover, therapeutic modulation of this gene, through the use of small molecule drugs, protein therapeutics or antibodies might be beneficial in the treatment of breast cancer.

In addition, there appears to be substantial expression of NOV11 associated with fetal lung (CT=30.3), when compared to expression in adult lung. Therefore, the expression of the NOV11 gene could be used to distinguish fetal lung tissue from adult lung tissue (CT=35.6).

Among tissues with metabolic function, the NOV11 gene is expressed in the pancreas (CT=33). Please see Panel 5 for discussion of utility of this gene in metabolic disease.

The NOV11 gene is also expressed at low levels in the hippocampus, cortex, thalamus, and amygdala. The NOV11 gene is a novel ionotropic purinergic receptor. These receptors play an important role in neuron excitatory transmission. In addition, all seizure disorders with genetic linkage currently known result from an ion channel mutation. The NOV11 gene is therefore an excellent small molecule target for the treatment of epilepsy or any seizure disorder, as well as any neuropsychiatric disease in which altered neurotransmission has been implicated (schizophrenia, bipolar disorder, or depression).

References:

Pankratov Y, Castro E, Miras-Portugal M T, Krishtal O. A purinergic component of the excitatory postsynaptic current mediated by P2X receptors in the CA1 neurons of the rat hippocampus. Eur J Neurosci Dec. 10, 1998 (12):3898-902

The pyramidal neurons in the CA1 area of hippocampal slices from 17- to 19-day-old rats have been investigated by means of patch clamp. Excitatory postsynaptic currents (EPSCs) were elicited by stimulating the Schaffer collateral at a frequency below 0.2 Hz. It was found that inhibition of glutamatergic transmission by 20 microM 6-cyano-7-nitroquinoxaline-2,3-dione (CNQX) and 100 microM 2-amino-5-phosphonovaleric acid (D-APV) left a small component of the EPSC uninhibited. The amplitude of this residual EPSC (rEPSC) comprised 25+/−11% of the total EPSC when measured at a holding potential of −50 mV. The rEPSC was blocked by selective P2 blocker pyridoxal phosphate-6-azophenyl-2′-4′-disulphonic acid (PPADS) 10 microM and bath incubation with non-hydrolysable ATP analogues, ATP-gamma-S and alpha, beta-methylene-ATP at 50 and 20 microM, respectively. The rEPSC was dramatically potentiated by external Zn2+(10 microM). In another series of experiments exogenous ATP was applied to the CA1 neurons in situ. An inward current evoked by ATP was inhibited by PPADS to the same extent as the rEPSC. It is concluded that, depending on membrane voltage, about one-fifth to one-quarter of the EPSC generated by the excitatory synaptic input to the hippocampal CA1 neurons of rat is due to the activity of P2X receptors.

Panel 5 Islet Summary: Ag3491 Expression of the NOV11 gene is limited to a sample derived from human islets of Langerhans (CT=33). This is in concordance with the expression seen in the pancreas in Panel 1.3D. Stimulation of P2 receptors with ligand enhances insulin secretion from islets. Therefore, an agonist for the P2 receptor homolog encoded by this gene may be a treatment for all types of Type 2 diabetes with beta cell secretory defects.

References:

Fernandez-Alvarez J, Hillaire-Buys D, Loubatieres-Mariani M M, Gomis R, Petit P. P2 receptor agonists stimulate insulin release from human pancreatic islets. Pancreas. January 2001; 22(1):69-71.

Although P2 receptors for adenosine 5′-triphosphate (ATP) and/or adenosine 5′-diphosphate (ADP) have been characterized in mammalian pancreatic beta cells, no evidence for an insulin-secreting effect of P2 receptor agonists has been reported as yet in humans. The present study aimed at investigating whether P2 receptor agonists could stimulate insulin release in human pancreatic islets obtained from brain-dead organ donors. Experiments were performed using different glucose concentrations and insulin was measured by radioimmunoassay. When the glucose concentration (8.3 mmol/L) was slightly stimulating for insulin release, alpha,beta-methylene ATP (200 micromol/L) and ADPbetaS (50 micromol/L) similarly amplified insulin secretion: both compounds induced a threefold increase in insulin response. In the presence of a nonstimulating glucose concentration (3.0 mmol/L), only alpha,beta-methylene ATP could induce a significant 1.4-fold increase in insulin release, ADPbetaS being completely ineffective. These results give evidence that P2 receptor agonists are effective in stimulating insulin release in humans, the effect of the P2Y agonist being essentially glucose dependent.

PMID: 11138974

K. NOV12: DIABLO-Like

Expression of the NOV12 gene (CG56132-01) was assessed using the primer-probe set Ag2884, described in Table 73. Results of the RTQ-PCR runs are shown in Tables 74, 75 and 76.

TABLE 73
Probe Name Ag2884

| | | | | SEQ ID |
|---|---|---|---|---|
| Primers | Sequences | Length | Start Position | NO: |
| Forward | 5′-tcctgaaagaatgttgtgcatt-3′ | 22 | 389 | 240 |
| Probe | TET-5′-tcttgaaagccaacttgatcctggta-3′-TAMRA | 26 | 411 | 241 |
| Reverse | 5′-accatatgtttctgcaaaacga-3′ | 22 | 453 | 242 |

TABLE 74
CNS_neurodegeneration_v1.0

| | Rel. Exp. (%) | | Rel. Exp. (%) |
|---|---|---|---|
| | Ag2884, Run | | Ag2884, Run |
| Tissue Name | 209058928 | Tissue Name | 209058928 |
| AD 1 Hippo | 5.9 | Control (Path) 3 Temporal | 2.2 |
| | | Ctx | |
| AD 2 Hippo | 19.6 | Control (Path) 4 Temporal | 22.5 |
| | | Ctx | |
| AD 3 Hippo | 3.0 | AD 1 Occipital Ctx | 8.4 |
| AD 4 Hippo | 3.1 | AD 2 Occipital Ctx (Missing) | 0.0 |
| AD 5 Hippo | 100.0 | AD 3 Occipital Ctx | 6.0 |
| AD 6 Hippo | 61.6 | AD 4 Occipital Ctx | 7.3 |
| Control 2 Hippo | 16.2 | AD 5 Occipital Ctx | 48.0 |
| Control 4 Hippo | 9.7 | AD 6 Occipital Ctx | 27.7 |
| Control (Path) 3 Hippo | 4.7 | Control 1 Occipital Ctx | 2.7 |
| AD 1 Temporal Ctx | 9.3 | Control 2 Occipital Ctx | 62.9 |
| AD 2 Temporal Ctx | 20.2 | Control 3 Occipital Ctx | 7.4 |
| AD 3 Temporal Ctx | 4.9 | Control 4 Occipital Ctx | 4.2 |
| AD 4 Temporal Ctx | 12.2 | Control (Path) 1 Occipital Ctx | 89.5 |
| AD 5 Inf Temporal Ctx | 88.3 | Control (Path) 2 Occipital Ctx | 7.6 |
| AD 5 Sup Temporal Ctx | 35.6 | Control (Path) 3 Occipital Ctx | 2.0 |
| AD 6 Inf Temporal Ctx | 65.5 | Control (Path) 4 Occipital Ctx | 14.5 |
| AD 6 Sup Temporal Ctx | 65.1 | Control 1 Parietal Ctx | 4.4 |
| Control 1 Temporal Ctx | 5.2 | Control 2 Parietal Ctx | 39.0 |
| Control 2 Temporal Ctx | 40.1 | Control 3 Parietal Ctx | 14.8 |
| Control 3 Temporal Ctx | 8.0 | Control (Path) 1 Parietal Ctx | 76.8 |
| Control 3 Temporal Ctx | 5.7 | Control (Path) 2 Parietal Ctx | 15.2 |
| Control (Path) 1 Temporal Ctx | 50.7 | Control (Path) 3 Parietal Ctx | 4.1 |
| Control (Path) 2 Temporal Ctx | 23.0 | Control (Path) 4 Parietal Ctx | 26.2 |

TABLE 75
Panel 1.3D

| | Rel. Exp. (%) | | Rel. Exp. (%) |
|---|---|---|---|
| | Ag2884, Run | | Ag2884, Run |
| Tissue Name | 167660112 | Tissue Name | 167660112 |
| Liver adenocarcinoma | 13.9 | Kidney (fetal) | 21.2 |
| Pancreas | 2.0 | Renal ca. 786-0 | 19.8 |
| Pancreatic ca. CAPAN 2 | 10.0 | Renal ca. A498 | 3.9 |
| Adrenal gland | 2.2 | Renal ca. RXF 393 | 8.2 |
| Thyroid | 1.7 | Renal ca. ACHN | 7.9 |
| Salivary gland | 2.8 | Renal ca. UO-31 | 4.8 |
| Pituitary gland | 5.8 | Renal ca. TK-10 | 14.6 |
| Brain (fetal) | 12.1 | Liver | 2.8 |
| Brain (whole) | 18.6 | Liver (fetal) | 3.2 |
| Brain (amygdala) | 9.3 | Liver ca. (hepatoblast) HepG2 | 8.1 |
| Brain (cerebellum) | 24.8 | Lung | 3.9 |
| Brain (hippocampus) | 6.8 | Lung (fetal) | 11.1 |
| Brain (substantia nigra) | 10.3 | Lung ca. (small cell) LX-1 | 11.7 |
| Brain (thalamus) | 7.2 | Lung ca. (small cell) NCI-H69 | 27.0 |
| Cerebral Cortex | 8.4 | Lung ca. (s. cell var.) SHP-77 | 100.0 |
| Spinal cord | 4.5 | Lung ca. (large cell) NCI-H460 | 4.1 |
| glio/astro U87-MG | 8.5 | Lung ca. (non-sm. cell) A549 | 39.5 |
| glio/astro U-118-MG | 11.6 | Lung ca. (non-s. cell) NCI-H23 | 15.5 |
| astrocytoma SW1783 | 14.1 | Lung ca. (non-s. cell) HOP-62 | 11.8 |
| neuro*; met SK-N-AS | 17.2 | Lung ca. (non-s. cl) NCI-H522 | 5.4 |
| astrocytoma SF-539 | 7.2 | Lung ca. (squam.) SW 900 | 30.8 |
| astrocytoma SNB-75 | 27.5 | Lung ca. (squam.) NCI-H596 | 31.6 |
| glioma SNB-19 | 8.0 | Mammary gland | 3.8 |
| glioma U251 | 27.5 | Breast ca.* (pl. ef) MCF-7 | 41.8 |
| glioma SF-295 | 17.1 | Breast ca.* (pl. ef) MDA-MB- | 7.7 |
| | | 231 | |
| Heart (fetal) | 4.6 | Breast ca.* (pl. ef) T47D | 58.6 |
| Heart | 3.8 | Breast ca. BT-549 | 3.1 |
| Skeletal muscle (fetal) | 3.6 | Breast ca. MDA-N | 6.0 |
| Skeletal muscle | 14.4 | Ovary | 3.3 |
| Bone marrow | 3.6 | Ovarian ca. OVCAR-3 | 17.7 |
| Thymus | 13.9 | Ovarian ca. OVCAR-4 | 7.8 |
| Spleen | 3.9 | Ovarian ca. OVCAR-5 | 85.3 |
| Lymph node | 8.0 | Ovarian ca. OVCAR-8 | 8.2 |
| Colorectal | 7.0 | Ovarian ca. IGROV-1 | 6.9 |
| Stomach | 4.9 | Ovarian ca.* (ascites) SK-OV- | 40.1 |
| | | 3 | |
| Small intestine | 4.8 | Uterus | 3.9 |
| Colon ca. SW480 | 6.9 | Placenta | 0.6 |
| Colon ca.* SW620 (SW480 met) | 28.7 | Prostate | 2.2 |
| Colon ca. HT29 | 6.0 | Prostate ca.* (bone met) PC-3 | 17.2 |
| Colon ca. HCT-116 | 7.4 | Testis | 2.0 |
| Colon ca. CaCo-2 | 9.0 | Melanoma Hs688(A).T | 3.6 |
| Colon ca. tissue (ODO3866) | 5.1 | Melanoma*; (met) Hs688(B).T | 7.0 |
| Colon ca. HCC-2998 | 17.7 | Melanoma UACC-62 | 13.5 |
| Gastric ca.* (liver met) NCI-N87 | 24.0 | Melanoma M14 | 1.7 |
| Bladder | 11.4 | Melanoma LOX IMVI | 5.2 |
| Trachea | 3.5 | Melanoma* (met) SK-MEL-5 | 8.3 |
| Kidney | 5.4 | Adipose | 11.6 |

TABLE 76
Panel 4D

| | Rel. Exp. (%) | | Rel. Exp. (%) |
|---|---|---|---|
| | Ag2884, Run | | Ag2884, Run |
| Tissue Name | 241701515 | Tissue Name | 241701515 |
| Secondary Th1 act | 18.8 | HUVEC IL-1beta | 5.7 |
| Secondary Th2 act | 38.2 | HUVEC IFN gamma | 12.4 |
| Secondary Tr1 act | 37.9 | HUVEC TNF alpha + IFN | 18.4 |
| | | gamma | |
| Secondary Th1 rest | 10.1 | HUVEC TNF alpha + IL4 | 14.3 |
| Secondary Th2 rest | 22.2 | HUVEC IL-11 | 3.8 |
| Secondary Tr1 rest | 16.4 | Lung Microvascular EC none | 24.7 |
| Primary Th1 act | 20.0 | Lung Microvascular EC | 14.8 |
| | | TNFalpha + IL-1beta | |
| Primary Th2 act | 29.5 | Microvascular Dermal EC none | 16.4 |
| Primary Tr1 act | 27.9 | Microsvasular Dermal EC | 14.1 |
| | | TNFalpha + IL-1beta | |
| Primary Th1 rest | 75.8 | Bronchial epithelium | 28.1 |
| | | TNFalpha + IL1beta | |
| Primary Th2 rest | 34.2 | Small airway epithelium none | 7.2 |
| Primary Tr1 rest | 32.3 | Small airway epithelium | 63.7 |
| | | TNFalpha + IL-1beta | |
| CD45RA CD4 lymphocyte act | 10.2 | Coronery artery SMC rest | 16.4 |
| CD45RO CD4 lymphocyte act | 25.7 | Coronery artery SMC | 8.1 |
| | | TNFalpha + IL-1beta | |
| CD8 lymphocyte act | 21.9 | Astrocytes rest | 15.0 |
| Secondary CD8 lymphocyte rest | 18.4 | Astrocytes TNFalpha + IL- | 19.8 |
| | | 1beta | |
| Secondary CD8 lymphocyte act | 21.0 | KU-812 (Basophil) rest | 24.3 |
| CD4 lymphocyte none | 26.4 | KU-812 (Basophil) | 99.3 |
| | | PMA/ionomycin | |
| 2ry Th1/Th2/Tr1_anti-CD95 | 45.1 | CCD1106 (Keratinocytes) none | 9.0 |
| CH11 | | | |
| LAK cells rest | 32.1 | CCD1106 (Keratinocytes) | 5.2 |
| | | TNFalpha + IL-1beta | |
| LAK cells IL-2 | 25.9 | Liver cirrhosis | 6.9 |
| LAK cells IL-2 + IL-12 | 53.2 | Lupus kidney | 5.3 |
| LAK cells IL-2 + IFN gamma | 60.7 | NCI-H292 none | 29.7 |
| LAK cells IL-2 + IL-18 | 37.6 | NCI-H292 IL-4 | 29.5 |
| LAK cells PMA/ionomycin | 26.6 | NCI-H292 IL-9 | 33.4 |
| NK Cells IL-2 rest | 42.2 | NCI-H292 IL-13 | 18.6 |
| Two Way MLR 3 day | 60.7 | NCI-H292 IFN gamma | 18.0 |
| Two Way MLR 5 day | 24.3 | HPAEC none | 7.3 |
| Two Way MLR 7 day | 19.9 | HPAEC TNF alpha + IL-1 beta | 14.9 |
| PBMC rest | 27.9 | Lung fibroblast none | 6.0 |
| PBMC PWM | 73.2 | Lung fibroblast TNF alpha + | 8.3 |
| | | IL-1 beta | |
| PBMC PHA-L | 32.1 | Lung fibroblast IL-4 | 21.6 |
| Ramos (B cell) none | 31.0 | Lung fibroblast IL-9 | 14.3 |
| Ramos (B cell) ionomycin | 100.0 | Lung fibroblast IL-13 | 13.6 |
| B lymphocytes PWM | 74.7 | Lung fibroblast IFN gamma | 26.8 |
| B lymphocytes CD40L and IL-4 | 29.3 | Dermal fibroblast CCD1070 | 32.8 |
| | | rest | |
| EOL-1 dbcAMP | 12.2 | Dermal fibroblast CCD1070 | 50.0 |
| | | TNF alpha | |
| EOL-1 dbcAMP PMA/ionomycin | 34.9 | Dermal fibroblast CCD1070 | 23.3 |
| | | IL-1 beta | |
| Dendritic cells none | 27.2 | Dermal fibroblast IFN gamma | 13.7 |
| Dendritic cells LPS | 31.0 | Dermal fibroblast IL-4 | 31.6 |
| Dendritic cells anti-CD40 | 23.7 | IBD Colitis 2 | 2.4 |
| Monocytes rest | 23.8 | IBD Crohn's | 2.5 |
| Monocytes LPS | 32.5 | Colon | 29.7 |
| Macrophages rest | 27.0 | Lung | 18.4 |
| Macrophages LPS | 31.9 | Thymus | 33.9 |
| HUVEC none | 12.2 | Kidney | 90.8 |
| HUVEC starved | 16.3 | | |

CNS_neurodegeneration_v1.0 Summary: Ag2884 No difference was found in the expression of the NOV12 gene in the postmortem brains of AD patients when compared to non-demented controls. This panel does demonstrate the expression of the NOV12 gene in the CNS of an independent group of patients. Please see panel 1.3D for a discussion of utility of this gene in the central nervous system.

Panel 1.3D Summary: Ag2884 The expression of the NOV12 gene, a diablo homolog, appears to be highest in a sample derived from a lung cancer cell line (SHP-77) (CT=30.4). In addition, there is substantial expression in other lung cancer cell lines, breast cancer cell lines and ovarian cancer cell lines. Thus, the expression of the NOV12 gene could be used to distinguish SHP-77 cells from other samples in the panel. Diablo activates caspases and promotes apoptosis. Mitochondria-mediated apoptosis plays a central role in animal development and tissue homeostasis, and its alteration results in a range of malignant disorders including cancer. Therefore, therapeutic modulation of the NOV12 gene, through the use of small molecule drugs, protein therapeutics or antibodies might be beneficial for the treatment of lung cancer, breast cancer or ovarian cancer.

The NOV12 gene is also expressed at low but significant levels in all CNS regions examined. Apoptosis has been implicated in Alzheimer's disease, traumatic brain injury, pathologic pain, stroke, viral infections of the CNS, Parkinson's disease, Huntington's disease, and multiple sclerosis. Therefore, the selective blockage/down regulation of the NOV12 gene or its protein product may have broad implications and utility in a number of CNS diseases/clinical conditions.

Among tissues with metabolic function, the NOV12 gene has low levels of expression in pituitary, fetal heart, skeletal muscle and adipose. Diablo proteins promote apoptosis by activating mitochondrial caspases. Therefore, inhibition of the NOV12 gene may protect against apoptosis/tissue wasting in diseases of the pituitary or skeletal muscle.

References:

Madesh M, Antonsson B, Srinivasula S M, Alnemri E S, Hajnoczky G. Rapid kinetics of tBid-induced cytochrome c and Smac/DIABLO release and mitochondrial depolarization.

J Biol Chem. Dec. 6, 2001 [epub ahead of print]

Cleavage of Bid has been shown to promote apoptosis by inducing mitochondrial membrane permeabilization with the resultant release of apoptosis-inducing proteins from the intermembrane space into the cytosol. However direct visualization of the Bid-induced release of various proteins from the highly compartmentalized intermembrane space and the changes in the mitochondrial metabolic machinery remain elusive. Using GFP fusion proteins and immunostaining in individual permeabilized HepG2 cells, first we demonstrated that truncated Bid (1 5.5-kDa C-terminal fragment, tBid) evoked a rapid and essentially complete release of cytochrome c and Smac/DIABLO from every mitochondrion. To establish at a resolution of seconds the kinetics of tBid-induced cytochrome c and Smac/DIABLO release and depolarization, we monitored the mitochondrial membrane potential fluorimetrically in permeabilized cells and applied a rapid filtration method to obtain cytosolic fractions for Western blotting. We found that subnanomolar doses of tBid were sufficient to evoke cytochrome c release and mitochondrial depolarization, whereas full-length Bid was 100-fold less effective. Bcl-xL prevented tBID-induced cytochrome c release and depolarization. In response to 2.5 nM tBid, cytochrome c release started after 10 s delay, displayed rapid progression and was complete at 50-70 s. Release of Smac/DIABLO was synchronized with cytochrome c release, whereas the loss of the mitochondrial membrane potential lagged slightly behind cytochrome c release. Furthermore, tBid-induced cytochrome c release was insensitive to changes in substrate composition, but tBid-induced depolarization did not occur in the presence of extramitochondrial ATP supply. Thus, tBID-induced permeabilization of the outer membrane permits rapid release of cytochrome c and Smac/DIABLO from all domains of the intermembrane space. The tBID-induced loss of mitochondrial membrane potential occurs after cytochrome c release and reflects impairment of oxidative metabolism.

PMID: 11741882

Adrain C, Creagh E M, Martin S J. Apoptosis-associated release of Smac/DIABLO from mitochondria requires active caspases and is blocked by Bcl-2. EMBO J. Dec. 3, 2001 ;20(23):6627-36.

Smac/DIABLO is a mitochondrial protein that potentiates some forms of apoptosis, possibly by neutralizing one or more members of the IAP family of apoptosis inhibitory proteins. Smac has been shown to exit mitochondria and enter the cytosol during apoptosis triggered by UV- or gamma-irradiation. Here, we report that Smac/DIABLO export from mitochondria into the cytosol is provoked by cytotoxic drugs and DNA damage, as well as by ligation of the CD95 death receptor. Mitochondrial efflux of Smac/DIABLO, in response to a variety of pro-apoptotic agents, was profoundly inhibited in Bcl-2-overexpressing cells. Thus, in addition to modulating apoptosis-associated mitochondrial cytochrome c release, Bcl-2 also regulates Smac release, suggesting that both molecules may escape via the same route. However, whereas cell stress-associated mitochondrial cytochrome c release was largely caspase independent, release of Smac/DIABLO in response to the same stimuli was blocked by a broad-spectrum caspase inhibitor. This suggests that apoptosis-associated cytochrome c and Smac/DIABLO release from mitochondria do not occur via the same mechanism. Rather, Smac/DIABLO efflux from mitochondria is a caspase-catalysed event that occurs downstream of cytochrome c release.

PMID: 11726499

Huang P, Oliff A. Signaling pathways in apoptosis as potential targets for cancer therapy. Trends Cell Biol August 2001;11(8):343-8

Genetic instability contributes to the origin of cancer as well as to the ability of cancer cells to become resistant to various therapies. Because of this, cytotoxic rather than cytostatic therapies might be most effective against this disease. Many oncogenes and tumor suppressors mediate their effects by interfering with or inducing apoptotic signaling. Thus, apoptotic pathways might be significantly altered in cancer cells relative to untransformed cells, and these differences might present a therapeutic window that can be exploited for development of cancer drugs.

PMID: 11489640

Panel 4D Summary: Ag2884 The expression of the NOV12 transcript is ubiquitous across panel 4D, with highest expression in kidney (CT=28.7), the basophil cell line Ku-812 and the B cell lymphoma cell line, Ramos, both upon treatment with ionomycin (CT 28.6). It is also moderately expressed in primary Th1 cells and PWM activated B cells but not in B cells treated with CD40L, a condition which was reported to promote survival. Moderate expression of the NOV12 transcript is also found in dermal fibroblasts and small airway epithelium treated with TNF-a whose cytotoxicity is well documented. The NOV12 transcript encodes for a Diablo like protein. Diablo proteins are pro-apoptotic mitochondrial proteins that are crucial for the activation of downstream effectors of apoptosis. Apoptosis has been implicated in the pathology of many autoimmune and inflammatory diseases. Therefore, modulation of the expression or activity of the NOV12 putative protein by small molecules may be beneficial for the treatment of rheumatoid arthritis, inflammatory bowel diseases, psoriasis, type 1 diabetes, lupus erythematosus and lung inflammatory diseases.

L. NOV13: HRPET-1 Related Protein

Expression of the NOV13 gene (CG56195-01) was assessed using the primer-probe set Ag2895, described in Table 77. Results of the RTQ-PCR runs are shown in Tables 78, 79, 80 and 81.

TABLE 77
Probe Name Ag2895

| Primers | Sequences | Length | Start Position | SEQ ID NO: |
|---|---|---|---|---|
| Forward | 5′-gcaggctttgaccttctca-3′ | 19 | 955 | 243 |
| Probe | TET-5′-acccggaagatgatcttgacccct-3′-TAMRA | 24 | 1010 | 244 |
| Reverse | 5′-ctgggacatgttcttctgtga-3′ | 21 | 1034 | 245 |

TABLE 78
CNS_neurodegeneration_v1.0

| | Rel. Exp. (%) | | Rel. Exp. (%) |
|---|---|---|---|
| | Ag2895, Run | | Ag2895, Run |
| Tissue Name | 224116296 | Tissue Name | 224116296 |
| AD 1 Hippo | 20.7 | Control (Path) 3 Temporal Ctx | 18.9 |
| AD 2 Hippo | 50.7 | Control (Path) 4 Temporal Ctx | 28.3 |
| AD 3 Hippo | 15.3 | AD 1 Occipital Ctx | 16.2 |
| AD 4 Hippo | 15.6 | AD 2 Occipital Ctx (Missing) | 0.0 |
| AD 5 hippo | 73.7 | AD 3 Occipital Ctx | 17.1 |
| AD 6 Hippo | 59.9 | AD 4 Occipital Ctx | 36.9 |
| Control 2 Hippo | 59.0 | AD 5 Occipital Ctx | 23.2 |
| Control 4 Hippo | 18.0 | AD 6 Occipital Ctx | 57.0 |
| Control (Path) 3 Hippo | 18.3 | Control 1 Occipital Ctx | 18.2 |
| AD 1 Temporal Ctx | 19.3 | Control 2 Occipital Ctx | 92.7 |
| AD 2 Temporal Ctx | 42.3 | Control 3 Occipital Ctx | 28.3 |
| AD 3 Temporal Ctx | 13.1 | Control 4 Occipital Ctx | 21.8 |
| AD 4 Temporal Ctx | 40.9 | Control (Path) 1 Occipital Ctx | 71.7 |
| AD 5 Inf Temporal Ctx | 100.0 | Control (Path) 2 Occipital Ctx | 21.6 |
| AD 5 Sup Temporal Ctx | 72.2 | Control (Path) 3 Occipital Ctx | 16.3 |
| AD 6 Inf Temporal Ctx | 46.0 | Control (Path) 4 Occipital Ctx | 14.7 |
| AD 6 Sup Temporal Ctx | 44.4 | Control 1 Parietal Ctx | 17.6 |
| Control 1 Temporal Ctx | 18.8 | Control 2 Parietal Ctx | 59.9 |
| Control 2 Temporal Ctx | 57.0 | Control 3 Parietal Ctx | 33.4 |
| Control 3 Temporal Ctx | 28.3 | Control (Path) 1 Parietal Ctx | 83.5 |
| Control 4 Temporal Ctx | 21.9 | Control (Path) 2 Parietal Ctx | 34.2 |
| Control (Path) 1 Temporal Ctx | 58.6 | Control (Path) 3 Parietal Ctx | 22.2 |
| Control (Path) 2 Temporal Ctx | 45.1 | Control (Path) 4 Parietal Ctx | 32.1 |

TABLE 79
Panel 1.3D

| | Rel. Exp. (%) | | Rel. Exp. (%) |
|---|---|---|---|
| | Ag2895, Run | | Ag2895, Run |
| Tissue Name | 167649468 | Tissue Name | 167649468 |
| Liver adenocarcinoma | 10.1 | Kidney (fetal) | 62.0 |
| Pancreas | 10.4 | Renal ca. 786-0 | 12.9 |
| Pancreatic ca. CAPAN2 | 6.6 | Renal ca. A498 | 26.6 |
| Adrenal gland | 5.8 | Renal ca. RXF 393 | 26.8 |
| Thyroid | 9.7 | Renal ca. ACHN | 10.4 |
| Salivary gland | 8.5 | Renal ca. UO-31 | 6.7 |
| Pituitary gland | 12.6 | Renal ca. TK-10 | 10.3 |
| Brain (fetal) | 21.0 | Liver | 12.4 |
| Brain (whole) | 13.5 | Liver (fetal) | 8.8 |
| Brain (amygdala) | 15.2 | Liver ca. (hepatoblast) HepG2 | 3.1 |
| Brain (cerebellum) | 16.5 | Lung | 16.8 |
| Brain (hippocampus) | 11.3 | Lung (fetal) | 15.7 |
| Brain (substantia nigra) | 5.4 | Lung ca. (small cell) LX-1 | 5.3 |
| Brain (thalamus) | 8.3 | Lung ca. (small cell) NCI-H69 | 5.3 |
| Cerebral Cortex | 32.3 | Lung ca. (s. cell var.) SHP-77 | 55.1 |
| Spinal cord | 9.3 | Lung ca. (large cell) NCI-H460 | 3.1 |
| glio/astro U87-MG | 19.8 | Lung ca. (non-sm. cell) A549 | 8.5 |
| glio/astro U-118-MG | 19.6 | Lung ca. (non-s. cell) NCI-H23 | 11.0 |
| astrocytoma SW1783 | 12.8 | Lung ca. (non-s. cell) HOP-62 | 13.4 |
| neuro*; met SK-N-AS | 5.3 | Lung ca. (non-s. cl) NCI-H522 | 5.6 |
| astrocytoma SF-539 | 9.4 | Lung ca. (squam.) SW 900 | 24.5 |
| astrocytoma SNB-75 | 26.2 | Lung ca. (squam.) NCI-H596 | 6.6 |
| Glioma SNB-19 | 2.7 | Mammary gland | 29.3 |
| Glioma U251 | 16.7 | Breast ca.* (pl. ef) MCF-7 | 9.8 |
| Glioma SF-295 | 27.7 | Breast ca.* (pl. ef) MDA-MB- | 13.2 |
| | | 231 | |
| Heart (fetal) | 51.8 | Breast ca.* (pl. ef) T47D | 19.1 |
| Heart | 9.1 | Breast ca. BT-549 | 4.4 |
| Skeletal muscle (fetal) | 17.0 | Breast ca. MDA-N | 15.2 |
| Skeletal muscle | 3.7 | Ovary | 28.9 |
| Bone marrow | 5.1 | Ovarian ca. OVCAR-3 | 13.1 |
| Thymus | 25.2 | Ovarian ca. OVCAR-4 | 17.3 |
| Spleen | 22.1 | Ovarian ca. OVCAR-5 | 100.0 |
| Lymph node | 19.3 | Ovarian ca. OVCAR-8 | 1.3 |
| Colorectal | 6.3 | Ovarian ca. IGROV-1 | 4.0 |
| Stomach | 6.9 | Ovarian ca.* (ascites) SK-OV- | 13.8 |
| | | 3 | |
| Small intestine | 9.5 | Uterus | 12.9 |
| Colon ca. SW480 | 9.8 | Placenta | 12.9 |
| Colon ca.* SW620 (SW480 met) | 7.3 | Prostate | 13.7 |
| Colon ca. HT29 | 7.2 | Prostate ca.* (bone met) PC-3 | 9.2 |
| Colon ca. HCT-116 | 4.2 | Testis | 16.7 |
| Colon ca. CaCo-2 | 11.7 | Melanoma Hs688(A).T | 8.5 |
| Colon ca. tissue (ODO3866) | 10.1 | Melanoma* (met) Hs688(B).T | 11.7 |
| Colon ca. HCC-2998 | 7.3 | Melanoma UACC-62 | 50.7 |
| Gastric ca.* (liver met) NCI-N87 | 20.4 | Melanoma M14 | 9.5 |
| Bladder | 6.8 | Melanoma LOX IMVI | 0.0 |
| Trachea | 16.2 | Melanoma* (met) SK-MEL-5 | 12.2 |
| Kidney | 27.0 | Adipose | 10.0 |

TABLE 80
Panel 2.2

| | Rel. Exp. (%) | | Rel. Exp. (%) |
|---|---|---|---|
| | Ag2895, Run | | Ag2895, Run |
| Tissue Name | 175119368 | Tissue Name | 175119368 |
| Normal Colon | 28.9 | Kidney Margin (OD04348) | 63.7 |
| Colon cancer (OD06064) | 25.7 | Kidney malignant cancer | 25.9 |
| | | (OD06204B) | |
| Colon Margin (OD06064) | 14.4 | Kidney normal adjacent tissue | 20.3 |
| | | (OD06204E) | |
| Colon cancer (OD06159) | 7.3 | Kidney Cancer (OD04450-01) | 26.1 |
| Colon Margin (OD06159) | 22.4 | Kidney Margin (OD04450-03) | 19.9 |
| Colon cancer (OD06297-04) | 7.9 | Kidney Cancer 8120613 | 7.9 |
| Colon Margin (OD06297-015) | 14.7 | Kidney Margin 8120614 | 31.9 |
| CC Gr. 2 ascend colon (ODO3921) | 9.4 | Kidney Cancer 9010320 | 11.0 |
| CC Margin (ODO3921) | 11.3 | Kidney Margin 9010321 | 10.4 |
| Colon cancer metastasis (OD06104) | 8.1 | Kidney Cancer 8120607 | 19.8 |
| Lung Margin (OD06104) | 13.9 | Kidney Margin 8120608 | 30.1 |
| Colon mets to lung (OD04451-01) | 21.8 | Normal Uterus | 23.2 |
| Lung Margin (OD04451-02) | 9.3 | Uterine Cancer 064011 | 5.4 |
| Normal Prostate | 27.5 | Normal Thyroid | 13.5 |
| Prostate Cancer (OD04410) | 10.4 | Thyroid Cancer 064010 | 20.4 |
| Prostate Margin (OD04410) | 14.7 | Thyroid Cancer A302152 | 27.7 |
| Normal Ovary | 52.1 | Thyroid Margin A302153 | 10.4 |
| Ovarian cancer (OD06283-03) | 21.2 | Normal Breast | 12.9 |
| Ovarian Margin (OD06283-07) | 10.8 | Breast Cancer (OD04566) | 7.6 |
| Ovarian Cancer 064008 | 18.6 | Breast Cancer 1024 | 50.0 |
| Ovarian cancer (OD06145) | 2.7 | Breast Cancer (OD04590-01) | 35.6 |
| Ovarian Margin (OD06145) | 10.4 | Breast Cancer Mets (OD04590- | 27.0 |
| | | 03) | |
| Ovarian cancer (OD06455-03) | 10.0 | Breast Cancer Metastasis | 100.0 |
| | | (OD04655-05) | |
| Ovarian Margin (OD06455-07) | 3.4 | Breast Cancer 064006 | 15.9 |
| Normal Lung | 20.6 | Breast Cancer 9100266 | 15.8 |
| Invasive poor diff. lung adeno | 12.3 | Breast Margin 9100265 | 4.8 |
| (ODO4945-01 | | | |
| Lung Margin (ODO4945-03) | 7.7 | Breast Cancer A209073 | 20.3 |
| Lung Malignant Cancer (OD03126) | 8.5 | Breast Margin A2090734 | 25.9 |
| Lung Margin (OD03126) | 7.0 | Breast cancer (OD06083) | 26.4 |
| Lung Cancer (OD05014A) | 30.1 | Breast cancer node metastasis | 16.4 |
| | | (OD06083) | |
| Lung Margin (OD05014B) | 6.2 | Normal Liver | 16.5 |
| Lung cancer (OD06081) | 29.1 | Liver Cancer 1026 | 21.9 |
| Lung Margin (OD06081) | 8.2 | Liver Cancer 1025 | 38.4 |
| Lung Cancer (OD04237-01) | 4.9 | Liver Cancer 6004-T | 27.5 |
| Lung Margin (OD04237-02) | 32.8 | Liver Tissue 6004-N | 2.0 |
| Ocular Melanoma Metastasis | 8.1 | Liver Cancer 6005-T | 30.6 |
| Ocular Melanoma Margin (Liver) | 17.8 | Liver Tissue 6005-N | 44.8 |
| Melanoma Metastasis | 17.7 | Liver Cancer 064003 | 26.8 |
| Melanoma Margin (Lung) | 18.6 | Normal Bladder | 14.7 |
| Normal Kidney | 10.7 | Bladder Cancer 1023 | 14.4 |
| Kidney Ca, Nuclear grade 2 | 36.6 | Bladder Cancer A302173 | 19.5 |
| (OD04338) | | | |
| Kidney Margin (OD04338) | 6.4 | Normal Stomach | 39.0 |
| Kidney Ca Nuclear grade 1/2 | 21.2 | Gastric Cancer 9060397 | 9.0 |
| (OD04339) | | | |
| Kidney Margin (OD04339) | 31.4 | Stomach Margin 9060396 | 16.5 |
| Kidney Ca, Clear cell type | 20.2 | Gastric Cancer 9060395 | 17.3 |
| (OD04340) | | | |
| Kidney Margin (OD04340) | 33.2 | Stomach Margin 9060394 | 27.5 |
| Kidney Ca, Nuclear grade 3 | 7.3 | Gastric Cancer 064005 | 15.3 |
| (OD04348) | | | |

TABLE 81
Panel 4D

| | Rel. Exp. (%) | | Rel. Exp. (%) |
|---|---|---|---|
| | Ag2895, Run | | Ag2895, Run |
| Tissue Name | 164310477 | Tissue Name | 164310477 |
| Secondary Th1 act | 41.8 | HUVEC IL-1beta | 5.5 |
| Secondary Th2 act | 43.2 | HUVEC IFN gamma | 17.0 |
| Secondary Tr1 act | 39.8 | HUVEC TNF alpha + IFN gamma | 29.9 |
| Secondary Th1 rest | 19.8 | HUVEC TNF alpha + IL4 | 34.6 |
| Secondary Th2 rest | 36.9 | HUVEC IL-11 | 6.7 |
| Secondary Tr1 rest | 39.8 | Lung Microvascular EC none | 20.7 |
| Primary Th1 act | 24.5 | Lung Microvascular EC | 45.7 |
| | | TNFalpha + IL-1beta | |
| Primary Th2 act | 33.2 | Microvascular Dermal EC none | 26.8 |
| Primary Tr1 act | 30.1 | Microsvasular Dermal EC | 41.8 |
| | | TNFalpha + IL-1beta | |
| Primary Th1 rest | 92.0 | Bronchial epithelium TNFalpha + | 72.2 |
| | | IL1beta | |
| Primary Th2 rest | 62.0 | Small airway epithelium none | 33.9 |
| Primary Tr1 rest | 55.9 | Small airway epithelium | 88.3 |
| | | TNFalpha + IL-1beta | |
| CD45RA CD4 lymphocyte act | 0.5 | Coronery artery SMC rest | 36.9 |
| CD45RO CD4 lymphocyte act | 29.7 | Coronery artery SMC TNFalpha + | 23.5 |
| | | IL-1beta | |
| CD8 lymphocyte act | 24.1 | Astrocytes rest | 52.1 |
| Secondary CD8 lymphocyte rest | 24.3 | Astrocytes TNFalpha + IL-1beta | 71.2 |
| Secondary CD8 lymphocyte act | 30.6 | KU-812 (Basophil) rest | 11.3 |
| CD4 lymphocyte none | 40.9 | KU-812 (Basophil) | 21.8 |
| | | PMA/ionomycin | |
| 2ry Th1/Th2/Tr1_anti-CD95 CH11 | 53.6 | CCD1106 (Keratinocytes) none | 34.6 |
| LAK cells rest | 35.1 | CCD1106 (Keratinocytes) | 54.7 |
| | | TNFalpha + IL-1beta | |
| LAK cells IL-2 | 34.9 | Liver cirrhosis | 12.6 |
| LAK cells IL-2 + IL-12 | 25.5 | Lupus kidney | 10.2 |
| LAK cells IL-2 + IFN gamma | 53.6 | NCI-H292 none | 18.3 |
| LAK cells IL-2 + IL-18 | 39.2 | NCI-H292 IL-4 | 12.7 |
| LAK cells PMA/ionomycin | 22.7 | NCI-H292 IL-9 | 12.1 |
| NK Cells IL-2 rest | 42.3 | NCI-H292 IL-13 | 11.5 |
| Two Way MLR 3 day | 37.1 | NCI-H292 IFN gamma | 17.9 |
| Two Way MLR 5 day | 23.7 | HPAEC none | 16.2 |
| Two Way MLR 7 day | 25.0 | HPAEC TNF alpha + IL-1 beta | 51.4 |
| PBMC rest | 40.3 | Lung fibroblast none | 22.1 |
| PBMC PWM | 54.3 | Lung fibroblast TNF alpha + IL-1 | 32.1 |
| | | beta | |
| PBMC PHA-L | 50.3 | Lung fibroblast IL-4 | 49.3 |
| Ramos (B cell) none | 14.2 | Lung fibroblast IL-9 | 32.3 |
| Ramos (B cell) ionomycin | 30.8 | Lung fibroblast IL-13 | 25.9 |
| B lymphocytes PWM | 64.2 | Lung fibroblast IFN gamma | 35.8 |
| B lymphocytes CD40L and IL-4 | 41.5 | Dermal fibroblast CCD1070 rest | 32.1 |
| EOL-1 dbcAMP | 3.7 | Dermal fibroblast CCD1070 TNF | 100.0 |
| | | alpha | |
| EOL-1 dbcAMP PMA/ionomycin | 12.1 | Dermal fibroblast CCD1070 IL-1 | 34.2 |
| | | beta | |
| Dendritic cells none | 34.4 | Dermal fibroblast IFN gamma | 25.0 |
| Dendritic cells LPS | 54.0 | Dermal fibroblast IL-4 | 26.4 |
| Dendritic cells anti-CD40 | 41.8 | IBD Colitis 2 | 3.9 |
| Monocytes rest | 47.0 | IBD Crohn's | 6.2 |
| Monocytes LPS | 21.2 | Colon | 21.5 |
| Macrophages rest | 40.3 | Lung | 24.3 |
| Macrophages LPS | 42.6 | Thymus | 59.5 |
| HUVEC none | 12.3 | Kidney | 81.2 |
| HUVEC starved | 21.5 | | |

CNS_neurodegeneration_v1.0 Summary: Ag2895 No difference is found in the expression of the NOV13 gene in the postmortem brains of AD patients when compared to non-demented controls. This panel does demonstrate the expression of the NOV13 gene in the CNS of an independent group of patients. Please see panel 1.3D for a discussion of utility of this gene in the central nervous system.

Panel 1.3D Summary: Ag2895 The expression of the NOV13 gene appears to be highest in a sample derived from an ovarian cancer cell line (OVCAR-5)(CT=28.5). There appears to be general lower levels of expression across the remainder of the panel. Thus, the expression of this gene could be used to distinguish OVCAR-5 cells from the rest of the samples in the panel.

This gene also has low but significant levels of expression in pancreas, adrenal, thyroid, pituitary, adult and fetal heart, skeletal muscle and liver, and adipose. Thus, this newly-identified gene product may be important for the pathogenesis, diagnosis and/or treatment of metabolic and endocrine diseases, including obesity and Types 1 and 2 diabetes.

This gene is expressed at moderate levels in all CNS regions examined. The NOV13 gene encodes a protein similar to EPI64 (ebp50-pdz interactor of 64 kd) that was reported to be a membrane associated protein that may be involved in cell adhesion and/or migration. In the CNS, these functions are usually associated with axon/dendritic growth and targeting. This molecule may therefore be of use in directing compensatory synaptogenesis in response to neuron death in spinal cord or brain trauma, stroke, Alzheimer's, Parkinson's or Huntington's diseases, or spinocerebellar ataxia.

References:

Reczek D, Bretscher A. Identification of EPI64, a TBC/rabGAP domain-containing microvillar protein that binds to the first PDZ domain of EBP50 and E3KARP. J Cell Biol Apr. 2, 2001;153(1):191-206

The cortical scaffolding proteins EBP50 (ERM-binding phosphoprotein-50) and E3KARP (NHE3 kinase A regulatory protein) contain two PDZ (PSD-95/DlgA/ZO-1-like) domains followed by a COOH-terminal sequence that binds to active ERM family members. Using affinity chromatography, we identified polypeptides from placental microvilli that bind the PDZ domains of EBP50. Among these are 64- and/or 65-kD differentially phosphorylated polypeptides that bind preferentially to the first PDZ domain of EBP50, as well as to E3KARP, and that we call EPI64 (EBP50-PDZ interactor of 64 kD). The gene for human EPI64 lies on chromosome 22 where nine exons specify a protein of 508 residues that contains a Tre/Bub2/Cdc16 (TBC)/rab GTPase-activating protein (GAP) domain. EPI64 terminates in DTYL, which is necessary for binding to the PDZ domains of EBP50, as a mutant ending in DTYLA no longer interacts. EPI64 colocalizes with EBP50 and ezrin in syncytiotrophoblast and cultured cell microvilli, and this localization in cultured cells is abolished by introduction of the DTYLA mutation. In addition to EPI64, immobilized EBP50 PDZ domains retain several polypeptides from placental microvilli, including an isoform of nadrin, a rhoGAP domain-containing protein implicated in regulating vesicular transport. Nadrin binds EBP50 directly probably through its COOH-terminal STAL sequence. Thus, EBP50 appears to bind membrane proteins as well as factors potentially involved in regulating membrane traffic.

PMID: 11285285

Panel 2.2 Summary: Ag2895 The expression of the NOV13 gene appears to be highest in a sample derived from a metastatic breast cancer (CT=30.8). Thus, the expression of this gene could be used to distinguish this breast cancer sample from the rest of the samples in the panel. Moreover, therapeutic modulation of the NOV13 gene, through the use of protein therapeutics, small molecule drugs or antibodies might be beneficial in the treatment of breast cancer.

Panel 4D Summary: Ag2895 The NOV13 transcript is expressed at high to moderate levels in most of the cells present in panel 4D. Highest expression of this transcript is found in dermal fibroblasts treated with TNF-a (CT=28.1), small airway epithelium and bronchial epithelium treated with TNF-a and IL-1b. It is also expressed at moderate levels in T and B cells. The NOV13 transcript encodes a HRPET-1 related protein, similar to ebp50-pdz interactor of 64 kd, which was reported to be a membrane associated protein that may be involved in cell adhesion and/or migration (see reference above). Therefore, modulation of the expression and/or activity of this putative protein by antibodies could block the functions of B and T cells and the interaction of these cells with local epithelium or fibroblasts. Consequently, this may reduce or eliminate the symptoms of chronic obstructive pulmonary disease, asthma, emphysema, bronchitis, psoriasis, inflammatory bowel disease, lupus erythematosus, and rheumatoid arthritis.

M. NOV14: B7-H2B

Expression of the NOV14 gene (CG55790-02) was assessed using the primer-probe sets Ag1845, Ag2589, Ag2621, Ag2915 and Ag210, described in Tables 82, 83, 84, 85 and 86. Results of the RTQ-PCR runs are shown in Tables 87, 88, 89, 90, 91, 92 and 93.

TABLE 82
Probe Name Ag1845

| Primers | Sequences | Length | Start Position | SEQ ID NO: |
|---|---|---|---|---|
| Forward | 5′-catcacgtgtgagctcacagt-3′ | 21 | 7454 | 246 |
| Probe | TET-5′-cttccacatggtgcactgctgct-3′-TAMRA | 23 | 7413 | 247 |
| Reverse | 5′-agaatttgcagacacagcaatt-3′ | 22 | 7386 | 248 |

TABLE 83
Probe Name Ag2589

| Primers | Sequences | Length | Start Position | SEQ ID NO: |
|---|---|---|---|---|
| Forward | 5′-gagctcaccttcacgtgtacat-3′ | 22 | 480 | 249 |
| Probe | TET-5′-ctaccccaggcccaacgtgtactg-3′-TAMRA | 24 | 512 | 250 |
| Reverse | 5′-gctgttgtccgtcttattgatc-3′ | 22 | 536 | 251 |

TABLE 84
Probe Name Ag2621

| Primers | Sequences | Length | Start Position | SEQ ID NO: |
|---|---|---|---|---|
| Forward | 5′-gagctcaccttcacgtgtacat-3′ | 22 | 480 | 252 |
| Probe | TET-5′-ctaccccaggcccaacgtgtactg-3′-TAMRA | 24 | 512 | 253 |
| Reverse | 5′-gctgttgtccgtcttattgatc-3′ | 22 | 536 | 254 |

TABLE 85
Probe Name Ag2915

| Primers | Sequences | Length | Start Position | SEQ ID NO: |
|---|---|---|---|---|
| Forward | 5′-gagctcaccttcacgtgtacat-3′ | 22 | 480 | 255 |
| Probe | TET-5′-ctaccccaggcccaacgtgtactg-3′-TAMRA | 24 | 512 | 256 |
| Reverse | 5′-gctgttgtccgtcttattgatc-3′ | 22 | 536 | 257 |

TABLE 86
Probe Name Ag210

| Primers | Sequences | Length | Start Position | SEQ ID NO: |
|---|---|---|---|---|
| Forward | 5′-gaggcagaggtcgcagtga-3′ | 19 | 7169 | 258 |
| Probe | TET-5′-tgcaccactgccctccagcct-3′-TAMRA | 21 | 7196 | 259 |
| Reverse | 5′-tttgagacggagtcttgctctgt-3′ | 23 | 7222 | 260 |

TABLE 87
AI_comprehensive panel_v1.0

| | Rel. Exp. (%) | | Rel. Exp. (%) |
|---|---|---|---|
| | Ag4845, Run | | Ag4845, Run |
| Tissue Name | 217699370 | Tissue Name | 217699370 |
| 110967 COPD-F | 8.5 | 112427 Match Control Psoriasis-F | 14.5 |
| 110980 COPD-F | 4.8 | 112418 Psoriasis-M | 6.6 |
| 110968 COPD-M | 6.7 | 112723 Match Control Psoriasis-M | 1.0 |
| 110977 COPD-M | 8.5 | 112419 Psoriasis-M | 9.9 |
| 110989 Emphysema-F | 12.9 | 112424 Match Control Psoriasis-M | 10.7 |
| 110992 Emphysema-F | 7.5 | 112420 Psoriasis-M | 21.5 |
| 110993 Emphysema-F | 8.5 | 112425 Match Control Psoriasis-M | 13.8 |
| 110994 Emphysema-F | 3.0 | 104689 (MF) OA Bone-Backus | 17.1 |
| 110995 Emphysema-F | 14.9 | 104690 (MF) Adj “Normal” Bone- | 6.8 |
| | | Backus | |
| 110996 Emphysema-F | 1.6 | 104691 (MF) OA Synovium-Backus | 14.6 |
| 110997 Asthma-M | 14.1 | 104692 (BA) OA Cartilage-Backus | 0.4 |
| 111001 Asthma-F | 7.9 | 104694 (BA) OA Bone-Backus | 13.3 |
| 111002 Asthma-F | 12.6 | 104695 (BA) Adj “Normal” Bone- | 9.3 |
| | | Backus | |
| 111003 Atopic Asthma-F | 13.4 | 104696 (BA) OA Synovium-Backus | 7.9 |
| 111004 Atopic Asthma-F | 16.5 | 104700 (SS) OA Bone-Backus | 7.5 |
| 111005 Atopic Asthma-F | 7.7 | 104701 (SS) Adj “Normal” Bone- | 7.7 |
| | | Backus | |
| 111006 Atopic Asthma-F | 0.9 | 104702 (SS) OA Synovium-Backus | 24.5 |
| 111417 Allergy-M | 6.3 | 117093 OA Cartilage Rep7 | 7.7 |
| 112347 Allergy-M | 1.7 | 112672 OA Bone5 | 6.2 |
| 112349 Normal Lung-F | 0.5 | 112673 OA Synovium5 | 1.1 |
| 112357 Normal Lung-F | 1.7 | 112674 OA Synovial Fluid cells5 | 2.6 |
| 112354 Normal Lung-M | 2.9 | 117100 OA Cartilage Rep14 | 4.3 |
| 112374 Crohns-F | 3.3 | 112756 OA Bone9 | 14.6 |
| 112389 Match Control Crohns- | 17.9 | 112757 OA Synovium9 | 7.1 |
| F | | | |
| 112375 Crohns-F | 1.8 | 112758 OA Synovial Fluid Cells9 | 4.2 |
| 112732 Match Control Crohns- | 39.5 | 117125 RA Cartilage Rep2 | 4.7 |
| F | | | |
| 112725 Crohns-M | 1.7 | 113492 Bone2 RA | 4.8 |
| 112387 Match Control Crohns- | 4.6 | 113493 Synovium2 RA | 3.6 |
| M | | | |
| 112378 Crohns-M | 0.7 | 113494 Syn Fluid Cells RA | 7.5 |
| 112390 Match Control Crohns- | 12.1 | 113499 Cartilage4 RA | 9.6 |
| M | | | |
| 112726 Crohns-M | 10.1 | 113500 Bone4 RA | 10.7 |
| 112731 Match Control Crohns- | 18.9 | 113501 Synovium4 RA | 6.9 |
| M | | | |
| 112380 Ulcer Col-F | 9.6 | 113502 Syn Fluid Cells4 RA | 5.4 |
| 112734 Match Control Ulcer | 100.0 | 113495 Cartilage3 RA | 4.0 |
| Col-F | | | |
| 112384 Ulcer Col-F | 17.1 | 113496 Bone3 RA | 7.2 |
| 112737 Match Control Ulcer | 13.5 | 113497 Synovium3 RA | 4.4 |
| Col-F | | | |
| 112386 Ulcer Col-F | 1.8 | 113498 Syn Fluid Cells3 RA | 6.2 |
| 112738 Match Control Ulcer | 1.4 | 117106 Normal Cartilage Rep20 | 7.3 |
| Col-F | | | |
| 112381 Ulcer Col-M | 7.3 | 113663 Bone3 Normal | 0.2 |
| 112735 Match Control Ulcer | 7.2 | 113664 Synovium3 Normal | 0.0 |
| Col-M | | | |
| 112382 Ulcer Col-M | 12.5 | 113665 Syn Fluid Cells3 Normal | 0.4 |
| 112394 Match Control Ulcer | 2.3 | 117107 Normal Cartilage Rep22 | 3.0 |
| Col-M | | | |
| 112383 Ulcer Col-M | 6.3 | 113667 Bone4 Normal | 4.3 |
| 112736 Match Control Ulcer | 13.0 | 113668 Synovium4 Normal | 2.7 |
| Col-M | | | |
| 112423 Psoriasis-F | 7.1 | 113669 Syn Fluid Cells4 Normal | 7.0 |

TABLE 88
CNS_neurodegeneration_v1.0

| | Rel. | Rel. | Rel. | Rel. | Rel. |
|---|---|---|---|---|---|
| | Exp. (%) | Exp. (%) | Exp. (%) | Exp. (%) | Exp. (%) |
| | Ag1845, | Ag1845, | Ag2589, | Ag2621, | Ag2915, |
| | Run | Run | Run | Run | Run |
| Tissue Name | 207807655 | 224079124 | 208776915 | 208393684 | 209735956 |
| AD 1 Hippo | 14.5 | 15.3 | 10.2 | 10.3 | 16.3 |
| AD 2 Hippo | 17.1 | 14.8 | 17.2 | 13.9 | 17.4 |
| AD 3 Hippo | 11.3 | 9.0 | 6.9 | 4.3 | 5.9 |
| AD 4 Hippo | 7.7 | 6.5 | 5.3 | 3.2 | 6.6 |
| AD 5 Hippo | 37.1 | 57.4 | 33.0 | 27.0 | 40.6 |
| AD 6 Hippo | 65.5 | 100.0 | 60.7 | 49.0 | 59.5 |
| Control 2 Hippo | 34.6 | 27.2 | 27.5 | 17.4 | 25.0 |
| Control 4 Hippo | 9.5 | 9.1 | 11.3 | 8.4 | 10.2 |
| Control (Path) 3 Hippo | 6.3 | 55.9 | 4.0 | 3.4 | 4.1 |
| AD 1 Temporal Ctx | 22.4 | 25.3 | 15.8 | 12.9 | 15.7 |
| AD 2 Temporal Ctx | 26.1 | 39.5 | 16.8 | 13.9 | 22.5 |
| AD 3 Temporal Ctx | 11.1 | 10.5 | 5.1 | 3.9 | 3.5 |
| AD 4 Temporal Ctx | 19.2 | 15.7 | 13.3 | 12.0 | 18.4 |
| AD 5 Inf Temporal Ctx | 93.3 | 75.3 | 66.9 | 59.5 | 84.7 |
| AD 5 Sup Temporal Ctx | 46.7 | 34.6 | 35.8 | 30.8 | 43.2 |
| AD 6 Inf Temporal Ctx | 100.0 | 39.8 | 100.0 | 100.0 | 100.0 |
| AD 6 Sup Temporal Ctx | 61.1 | 58.2 | 50.3 | 35.6 | 52.1 |
| Control 1 Temporal Ctx | 3.2 | 5.3 | 4.0 | 2.4 | 3.8 |
| Control 2 Temporal Ctx | 22.5 | 31.6 | 20.6 | 18.2 | 7.5 |
| Control 3 Temporal Ctx | 9.2 | 13.8 | 8.3 | 5.8 | 7.7 |
| Control 3 Temporal Ctx | 7.6 | 3.1 | 5.1 | 4.2 | 9.2 |
| Control (Path) 1 Temporal Ctx | 25.2 | 47.3 | 25.5 | 17.6 | 26.4 |
| Control (Path) 2 Temporal Ctx | 11.9 | 10.2 | 13.0 | 11.5 | 12.6 |
| Control (Path) 3 Temporal Ctx | 5.7 | 6.0 | 1.9 | 1.4 | 1.8 |
| Control (Path) 4 Temporal Ctx | 11.8 | 47.0 | 10.2 | 8.5 | 11.7 |
| AD 1 Occipital Ctx | 13.5 | 8.1 | 9.9 | 6.3 | 11.1 |
| AD 2 Occipital Ctx (Missing) | 0.0 | 0.0 | 0.0 | 0.4 | 0.0 |
| AD 3 Occipital Ctx | 6.9 | 8.5 | 4.5 | 3.8 | 5.9 |
| AD 4 Occipital Ctx | 23.3 | 12.7 | 14.5 | 10.8 | 14.1 |
| AD 5 Occipital Ctx | 25.2 | 62.9 | 21.0 | 16.7 | 21.3 |
| AD 6 Occipital Ctx | 24.5 | 36.9 | 18.9 | 15.5 | 21.0 |
| Control 1 Occipital Ctx | 5.8 | 6.8 | 3.5 | 2.4 | 2.7 |
| Control 2 Occipital Ctx | 34.9 | 26.6 | 24.8 | 25.5 | 36.9 |
| Control 3 Occipital Ctx | 10.0 | 14.5 | 9.0 | 5.8 | 9.0 |
| Control 4 Occipital Ctx | 10.2 | 13.5 | 5.1 | 5.6 | 7.1 |
| Control (Path) 1 Occipital Ctx | 56.6 | 55.9 | 53.6 | 42.3 | 56.6 |
| Control (Path) 2 Occipital Ctx | 6.5 | 23.7 | 7.8 | 6.3 | 11.2 |
| Control (Path) 3 Occipital Ctx | 4.3 | 2.9 | 2.3 | 2.7 | 2.2 |
| Control (Path) 4 Occipital Ctx | 10.8 | 13.5 | 9.9 | 8.1 | 9.9 |
| Control 1 Parietal Ctx | 10.4 | 8.2 | 7.5 | 6.2 | 6.7 |
| Control 2 Parietal Ctx | 40.9 | 32.1 | 31.4 | 22.2 | 30.1 |
| Control 3 Parietal Ctx | 16.3 | 27.2 | 11.4 | 8.9 | 13.6 |
| Control (Path) 1 Parietal Ctx | 29.9 | 24.3 | 29.1 | 23.5 | 29.1 |
| Control (Path) 2 Parietal Ctx | 14.4 | 24.5 | 11.6 | 9.6 | 17.6 |
| Control (Path) 3 Parietal Ctx | 3.4 | 7.3 | 2.9 | 1.9 | 1.8 |
| Control (Path) 4 Parietal Ctx | 19.5 | 18.4 | 18.6 | 16.3 | 18.8 |

TABLE 89
Panel 1

| | Rel. Exp. (%) | | Rel. Exp. (%) |
|---|---|---|---|
| | Ag210, Run | | Ag210, Run |
| Tissue Name | 87987363 | Tissue Name | 87987363 |
| Endothelial cells | 5.5 | Renal ca. 786-0 | 2.5 |
| Endothelial cells (treated) | 0.4 | Renal ca. A498 | 0.7 |
| Pancreas | 17.1 | Renal ca. RXF 393 | 0.7 |
| Pancreatic ca. CAPAN2 | 0.5 | Renal ca. ACHN | 0.1 |
| Adrenal gland | 11.8 | Renal ca. UO-31 | 1.3 |
| Thyroid | 0.7 | Renal ca. TK-10 | 0.0 |
| Salivary gland | 2.3 | Liver | 13.6 |
| Pituitary gland | 1.1 | Liver (fetal) | 2.1 |
| Brain (fetal) | 14.8 | Liver ca. (hepatoblast) HepG2 | 0.0 |
| Brain (whole) | 36.6 | Lung | 89.5 |
| Brain (amygdala) | 9.8 | Lung (fetal) | 50.3 |
| Brain (cerebellum) | 100.0 | Lung ca. (small cell) LX-1 | 0.1 |
| Brain (hippocampus) | 33.0 | Lung ca. (small cell) NCI-H69 | 3.4 |
| Brain (substantia nigra) | 4.2 | Lung ca. (s. cell var.) SHP-77 | 0.0 |
| Brain (thalamus) | 7.1 | Lung ca. (large cell) NCI-H460 | 0.0 |
| Brain (hypothalamus) | 7.6 | Lung ca. (non-sm. cell) A549 | 1.2 |
| Spinal cord | 3.4 | Lung ca. (non-s. cell) NCI-H23 | 1.5 |
| Glio/astro U87-MG | 1.4 | Lung ca. (non-s. cell) HOP-62 | 0.1 |
| Glio/astro U-118-MG | 9.7 | Lung ca. (non-s. cl) NCI-H522 | 0.1 |
| astrocytoma SW1783 | 2.0 | Lung ca. (squam.) SW 900 | 1.8 |
| neuro*; met SK-N-AS | 0.6 | Lung ca. (squam.) NCI-H596 | 4.9 |
| astrocytoma SF-539 | 0.4 | Mammary gland | 18.7 |
| astrocytoma SNB-75 | 0.7 | Breast ca.* (pl. ef) MCF-7 | 3.3 |
| glioma SNB-19 | 1.7 | Breast ca.* (pl. ef) MDA-MB- | 1.2 |
| | | 231 | |
| glioma U251 | 0.6 | Breast ca.* (pl. ef) T47D | 12.2 |
| glioma SF-295 | 0.0 | Breast ca. BT-549 | 0.0 |
| Heart | 3.6 | Breast ca. MDA-N | 5.6 |
| Skeletal muscle | 1.0 | Ovary | 0.1 |
| Bone marrow | 13.2 | Ovarian ca. OVCAR-3 | 0.7 |
| Thymus | 12.8 | Ovarian ca. OVCAR-4 | 0.1 |
| Spleen | 2.5 | Ovarian ca. OVCAR-5 | 0.7 |
| Lymph node | 6.7 | Ovarian ca. OVCAR-8 | 1.2 |
| Colon (ascending) | 20.0 | Ovarian ca. IGROV-1 | 0.3 |
| Stomach | 6.9 | Ovarian ca. (ascites) SK-OV-3 | 1.2 |
| Small intestine | 2.6 | Uterus | 10.5 |
| Colon ca SW480 | 0.2 | Placenta | 15.4 |
| Colon ca.* SW620 (SW480 met) | 0.2 | Prostate | 9.0 |
| Colon ca. HT29 | 1.3 | Prostate ca.* (bone met) PC-3 | 0.0 |
| Colon ca. HCT-116 | 0.0 | Testis | 9.5 |
| Colon ca. CaCo-2 | 4.5 | Melanoma Hs688(A).T | 3.2 |
| Colon ca. HCT-15 | 3.3 | Melanoma* (met) Hs688(B).T | 2.5 |
| Colon ca. HCC-2998 | 3.8 | Melanoma UACC-62 | 0.0 |
| Gastric ca. * (liver met) NCI-N87 | 3.0 | Melanoma M14 | 2.6 |
| Bladder | 6.5 | Melanoma LOX IMVI | 18.0 |
| Trachea | 15.1 | Melanoma* (met) SK-MEL-5 | 0.0 |
| Kidney | 13.0 | Melanoma SK-MEL-28 | 4.5 |
| Kidney (fetal) | 13.1 | | |

TABLE 90
Panel 1.3D

| | Rel. | Rel. | Rel. | Rel. | Rel. |
|---|---|---|---|---|---|
| | Exp. (%) | Exp. (%) | Exp. (%) | Exp. (%) | Exp. (%) |
| | Ag1845, | Ag1845, | Ag2589, | Ag2621, | Ag2915, |
| | Run | Run | Run | Run | Run |
| Tissue Name | 148648438 | 149951496 | 167660070 | 167644903 | 167646705 |
| Liver adenocarcinoma | 3.9 | 4.7 | 23.8 | 17.6 | 22.8 |
| Pancreas | 0.9 | 0.3 | 0.8 | 1.6 | 2.3 |
| Pancreatic ca. CAPAN2 | 0.0 | 0.0 | 2.6 | 0.5 | 1.0 |
| Adrenal gland | 1.6 | 2.1 | 3.7 | 3.2 | 1.9 |
| Thyroid | 2.2 | 2.9 | 4.2 | 5.3 | 3.3 |
| Salivary gland | 2.0 | 3.1 | 6.3 | 4.0 | 4.2 |
| Pituitary gland | 0.9 | 1.1 | 2.0 | 2.0 | 1.4 |
| Brain (fetal) | 0.2 | 0.9 | 4.6 | 6.0 | 3.3 |
| Brain (whole) | 31.2 | 31.4 | 90.8 | 90.8 | 77.4 |
| Brain (amygdala) | 28.5 | 24.7 | 34.9 | 31.4 | 34.4 |
| Brain (cerebellum) | 3.0 | 1.5 | 21.6 | 18.6 | 20.7 |
| Brain (hippocampus) | 100.0 | 100.0 | 39.2 | 29.9 | 27.7 |
| Brain (substantia nigra) | 26.1 | 21.3 | 86.5 | 60.7 | 67.8 |
| Brain (thalamus) | 29.9 | 31.0 | 89.5 | 49.7 | 59.0 |
| Cerebral Cortex | 20.0 | 16.4 | 46.3 | 33.2 | 38.4 |
| Spinal cord | 15.0 | 12.7 | 29.9 | 20.2 | 31.0 |
| Glio/astro U87-MG | 2.6 | 3.5 | 18.7 | 13.3 | 18.9 |
| Glio/astro U-118-MG | 1.3 | 0.3 | 0.6 | 1.6 | 1.6 |
| astrocytoma SW1783 | 0.0 | 0.1 | 1.5 | 1.0 | 0.3 |
| neuro*; met SK-N-AS | 0.0 | 0.0 | 0.0 | 0.0 | 0.0 |
| astrocytoma SF-539 | 5.5 | 4.2 | 28.9 | 15.3 | 21.8 |
| astrocytoma SNB-75 | 1.9 | 1.3 | 10.7 | 5.4 | 5.5 |
| glioma SNB-19 | 0.6 | 1.6 | 2.4 | 1.7 | 3.6 |
| glioma 251 | 0.4 | 0.9 | 10.0 | 7.1 | 5.0 |
| glioma SF-295 | 11.3 | 12.1 | 24.1 | 17.1 | 25.5 |
| Heart (fetal) | 12.9 | 8.2 | 29.3 | 24.5 | 31.4 |
| Heart | 1.9 | 2.0 | 12.8 | 8.5 | 12.2 |
| Skeletal muscle (fetal) | 54.7 | 48.6 | 30.6 | 34.9 | 36.1 |
| Skeletal muscle | 1.0 | 0.6 | 3.1 | 4.4 | 3.2 |
| Bone marrow | 3.2 | 2.6 | 3.3 | 3.5 | 3.6 |
| Thymus | 4.9 | 5.3 | 10.2 | 11.1 | 11.3 |
| Spleen | 15.9 | 15.8 | 11.2 | 10.7 | 15.3 |
| Lymph node | 11.4 | 11.0 | 27.0 | 29.5 | 28.7 |
| Colorectal | 4.5 | 6.5 | 9.5 | 8.1 | 7.5 |
| Stomach | 9.2 | 8.5 | 9.5 | 8.2 | 9.7 |
| Small intestine | 5.7 | 6.4 | 6.8 | 4.6 | 5.7 |
| Colon ca. SW480 | 2.0 | 4.7 | 7.3 | 6.3 | 7.9 |
| Colon ca.* | 0.8 | 2.1 | 12.2 | 26.4 | 19.2 |
| SW620(SW480 met) | | | | | |
| Colon ca. HT29 | 0.3 | 0.4 | 30.8 | 4.2 | 4.3 |
| Colon ca. HCT-116 | 4.9 | 6.5 | 12.2 | 14.7 | 14.2 |
| Colon ca. CaCo-2 | 15.1 | 12.4 | 30.8 | 28.5 | 29.7 |
| Colon ca. | 5.9 | 5.6 | 17.3 | 24.3 | 19.3 |
| tissue(ODO3866) | | | | | |
| Colon ca. HCC-2998 | 5.0 | 9.9 | 30.8 | 31.9 | 35.8 |
| Gastric ca.* (liver met) | 3.3 | 6.0 | 6.1 | 7.4 | 6.2 |
| NCI-N87 | | | | | |
| Bladder | 2.1 | 1.9 | 14.3 | 9.7 | 15.7 |
| Trachea | 9.7 | 5.7 | 3.0 | 2.3 | 2.3 |
| Kidney | 5.5 | 3.9 | 24.0 | 23.2 | 21.9 |
| Kidney (fetal) | 11.8 | 14.4 | 100.0 | 100.0 | 85.9 |
| Renal ca. 786-0 | 6.5 | 5.2 | 27.4 | 28.3 | 33.2 |
| Renal ca. A498 | 8.2 | 9.6 | 19.3 | 21.5 | 21.8 |
| Renal ca. RXF 393 | 3.5 | 3.6 | 50.0 | 55.9 | 48.6 |
| Renal ca. ACHN | 3.2 | 4.7 | 8.7 | 7.6 | 9.2 |
| Renal ca. UO-31 | 3.0 | 50.0 | 3.3 | 4.2 | 4.2 |
| Renal ca. TK-10 | 4.4 | 4.9 | 18.0 | 13.7 | 15.5 |
| Liver | 3.2 | 1.2 | 5.6 | 4.4 | 8.0 |
| Liver (fetal) | 2.7 | 2.5 | 1.9 | 5.6 | 2.6 |
| Liver ca. (hepatoblast) | 1.9 | 3.0 | 10.8 | 8.7 | 9.6 |
| HepG2 | | | | | |
| Lung | 7.5 | 7.7 | 12.8 | 13.9 | 17.0 |
| Lung (fetal) | 7.0 | 8.2 | 13.3 | 6.8 | 5.0 |
| Lung ca. (small cell) | 2.6 | 2.3 | 9.0 | 6.1 | 9.6 |
| LX-1 | | | | | |
| Lung ca. (small cell) | 0.7 | 0.3 | 1.5 | 1.0 | 0.5 |
| NCI-H69 | | | | | |
| Lung ca. (s. cell var.) | 0.2 | 0.0 | 0.0 | 0.0 | 0.5 |
| SHP-77 | | | | | |
| Lung ca. (large | 0.5 | 1.1 | 0.6 | 0.0 | 0.7 |
| cell) NCI-H460 | | | | | |
| Lung ca. (non-sm. Cell) | 0.3 | 0.2 | 4.0 | 4.0 | 5.7 |
| A549 | | | | | |
| Lung ca. (non-s. cell) | 4.0 | 3.3 | 8.4 | 6.6 | 7.8 |
| NCI-H23 | | | | | |
| Lung ca. (non-s. cell) | 1.5 | 1.7 | 1.5 | 2.4 | 3.3 |
| HOP-62 | | | | | |
| Lung ca. (non-s. cl) NCI- | 4.0 | 8.4 | 13.4 | 12.3 | 12.0 |
| H522 | | | | | |
| Lung ca. (squam.) SW | 2.0 | 0.4 | 5.9 | 5.8 | 4.6 |
| 900 | | | | | |
| Lung ca. (squam.) NCI- | 0.0 | 0.0 | 0.0 | 0.4 | 1.1 |
| H596 | | | | | |
| Mammary gland | 12.3 | 7.6 | 32.1 | 26.2 | 32.1 |
| Breast ca.* (pl. ef) MCF-7 | 15.3 | 10.5 | 76.3 | 79.0 | 100.0 |
| Breast ca.* (pl. ef) | 1.8 | 2.0 | 6.2 | 6.7 | 6.5 |
| MDA-MB-231 | | | | | |
| Breast ca.* (pl. ef) T47D | 4.5 | 6.4 | 35.8 | 31.9 | 37.4 |
| Breast ca. BT-549 | 0.9 | 1.4 | 9.1 | 6.3 | 6.2 |
| Breast ca. MDA-N | 0.6 | 0.9 | 2.9 | 4.3 | 6.5 |
| Ovary | 2.2 | 4.0 | 5.0 | 6.3 | 6.3 |
| Ovarian ca. OVCAR-3 | 8.7 | 6.9 | 26.2 | 31.6 | 41.2 |
| Ovarian ca. OVCAR-4 | 1.6 | 1.9 | 23.8 | 11.5 | 20.2 |
| Ovarian ca. OVCAR-5 | 1.9 | 2.6 | 20.7 | 17.6 | 14.7 |
| Ovarian ca. OVCAR-8 | 0.9 | 1.8 | 2.5 | 2.7 | 1.3 |
| Ovarian ca. IGROV-1 | 1.9 | 1.0 | 10.7 | 8.1 | 9.9 |
| Ovarian ca.* (ascites) | 0.5 | 1.2 | 16.7 | 12.0 | 10.7 |
| SK-OV-3 | | | | | |
| Uterus | 1.9 | 3.4 | 2.4 | 4.2 | 4.1 |
| Placenta | 1.6 | 1.6 | 1.8 | 1.5 | 1.4 |
| Prostate | 7.6 | 3.2 | 4.6 | 2.9 | 3.8 |
| Prostate ca.* (bone | 6.5 | 7.0 | 19.9 | 17.2 | 19.3 |
| met)PC-3 | | | | | |
| Testis | 4.0 | 3.3 | 3.0 | 1.0 | 1.9 |
| Melanoma Hs688(A).T | 0.0 | 0.0 | 0.0 | 0.0 | 0.0 |
| Melanoma* (met) | 0.0 | 0.0 | 0.0 | 0.0 | 0.0 |
| Hs688(B).T | | | | | |
| Melanoma UACC-62 | 0.2 | 0.2 | 1.0 | 3.6 | 3.8 |
| Melanoma M14 | 0.0 | 0.0 | 0.0 | 0.0 | 0.0 |
| Melanoma LOX IMVI | 0.5 | 0.4 | 0.0 | 0.0 | 0.0 |
| Melanoma* (met) SK- | 1.1 | 0.2 | 1.3 | 2.2 | 2.4 |
| MEL-5 | | | | | |
| Adipose | 9.7 | 11.0 | 32.8 | 29.9 | 31.4 |

TABLE 91
Panel 2.2

| | Rel. Exp. (%) | | Rel. Exp. (%) |
|---|---|---|---|
| | Ag2621, Run | | Ag2621, Run |
| Tissue Name | 175063689 | Tissue Name | 175063689 |
| Normal Colon | 6.5 | Kidney Margin (OD04348) | 100.0 |
| Colon cancer (OD06064) | 8.7 | Kidney malignant cancer | 12.3 |
| | | (OD06204B) | |
| Colon Margin (OD06064) | 6.9 | Kidney normal adjacent tissue | 18.9 |
| | | (OD06204E) | |
| Colon cancer (OD06159) | 2.1 | Kidney Cancer (OD04450-01) | 6.7 |
| Colon Margin (OD06159) | 5.9 | Kidney Margin (OD04450-03) | 12.9 |
| Colon cancer (OD06297-04) | 3.1 | Kidney Cancer 8120613 | 5.8 |
| Colon Margin (OD06297-015) | 9.7 | Kidney Margin 8120614 | 32.8 |
| CC Gr. 2 ascend colon (ODO3921) | 10.8 | Kidney Cancer 9010320 | 13.8 |
| CC Margin (ODO3921) | 4.1 | Kidney Margin 9010321 | 14.9 |
| Colon cancer metastasis | 6.6 | Kidney Cancer 8120607 | 16.7 |
| (OD06104) | | | |
| Lung Margin (OD06104) | 6.0 | Kidney Margin 8120608 | 10.4 |
| Colon mets to lung (OD04451-01) | 9.9 | Normal Uterus | 9.0 |
| Lung Margin (OD04451-02) | 5.6 | Uterine Cancer 064011 | 4.7 |
| Normal Prostate | 4.7 | Normal Thyroid | 0.7 |
| Prostate Cancer (OD04410) | 2.1 | Thyroid Cancer 064010 | 10.1 |
| Prostate Margin (OD04410) | 4.5 | Thyroid Cancer A302152 | 3.9 |
| Normal Ovary | 2.5 | Thyroid Margin A302153 | 1.2 |
| Ovarian cancer (OD06283-03) | 19.3 | Normal Breast | 10.9 |
| Ovarian Margin (OD06283-07) | 7.6 | Breast Cancer (OD04566) | 9.5 |
| Ovarian Cancer 064008 | 5.6 | Breast Cancer 1024 | 28.3 |
| Ovarian cancer (OD06145) | 6.5 | Breast Cancer (OD04590-01) | 32.3 |
| Ovarian Margin (OD06145) | 11.7 | Breast Cancer Mets (OD04590- | 13.6 |
| | | 03) | |
| Ovarian cancer (OD06455-03) | 4.1 | Breast Cancer Metastasis | 12.9 |
| | | (OD04655-05) | |
| Ovarian Margin (OD06455-07) | 5.6 | Breast Cancer 064006 | 12.9 |
| Normal Lung | 14.6 | Breast Cancer 9100266 | 5.8 |
| Invasive poor diff. lung adeno | 3.8 | Breast Margin 9100265 | 7.8 |
| (ODO4945-01 | | | |
| Lung Margin (ODO4945-03) | 6.3 | Breast Cancer A209073 | 4.7 |
| Lung Malignant Cancer | 4.2 | Breast Margin A2090734 | 23.3 |
| (OD03126) | | | |
| Lung Margin (OD03126) | 6.7 | Breast cancer (OD06083) | 23.5 |
| Lung Cancer (OD05014A) | 5.9 | Breast cancer node metastasis | 15.8 |
| | | (OD06083) | |
| Lung Margin (OD05014B) | 8.5 | Normal Liver | 23.2 |
| Lung cancer (OD06081) | 5.5 | Liver Cancer 1026 | 5.6 |
| Lung Margin (OD06081) | 3.5 | Liver Cancer 1025 | 13.6 |
| Lung Cancer (OD04237-01) | 3.0 | Liver Cancer 6004-T | 19.1 |
| Lung Margin (OD04237-02) | 17.4 | Liver Tissue 6004-N | 1.4 |
| Ocular Melanoma Metastasis | 3.2 | Liver Cancer 6005-T | 19.2 |
| Ocular Melanoma Margin (Liver) | 9.7 | Liver Tissue 6005-N | 18.3 |
| Melanoma Metastasis | 1.4 | Liver Cancer 064003 | 2.2 |
| Melanoma Margin (Lung) | 5.3 | Normal Bladder | 16.2 |
| Normal Kidney | 10.6 | Bladder Cancer 1023 | 8.2 |
| Kidney Ca, Nuclear grade 2 | 45.7 | Bladder Cancer A302173 | 27.4 |
| (OD04338) | | | |
| Kidney Margin (OD04338) | 10.6 | Normal Stomach | 18.4 |
| Kidney Ca Nuclear grade ½ | 33.2 | Gastric Cancer 9060397 | 17.0 |
| (OD04339) | | | |
| Kidney Margin (OD04339) | 23.0 | Stomach Margin 9060396 | 7.5 |
| Kidney Ca, Clear cell type | 47.3 | Gastric Cancer 9060395 | 5.7 |
| (OD04340) | | | |
| Kidney Margin (OD04340) | 14.7 | Stomach Margin 9060394 | 13.6 |
| Kidney Ca, Nuclear grade 3 | 6.0 | Gastric Cancer 064005 | 11.3 |
| (OD04348) | | | |

TABLE 92
Panel 2D

| | | Rel. | | Rel. | Rel. |
|---|---|---|---|---|---|
| | Rel. | Exp. (%) | | Exp. (%) | Exp. (%) |
| | Exp. (%) Ag1845, | Ag1845, | | Ag1845, | Ag1845, |
| | Run | Run | | Run | Run |
| Tissue Name | 148648439 | 149957753 | Tissue Name | 148648439 | 149957753 |
| Normal Colon | 35.1 | 24.5 | Kidney Margin 8120608 | 44.4 | 28.3 |
| CC Well to Mod Diff | 19.3 | 16.6 | Kidney Cancer 8120613 | 30.4 | 17.8 |
| (ODO3866) | | | | | |
| CC Margin (ODO3866) | 9.4 | 11.9 | Kidney Margin 8120614 | 62.9 | 51.8 |
| CC Gr. 2 rectosigmoid | 14.0 | 11.7 | Kidney Cancer 9010320 | 36.3 | 26.4 |
| (ODO3868) | | | | | |
| CC Margin (ODO3868) | 1.4 | 1.9 | Kidney Margin 9010321 | 46.7 | 38.2 |
| CC Mod Diff (ODO3920) | 9.6 | 7.1 | Normal Uterus | 2.1 | 3.8 |
| CC Margin (ODO3920) | 6.1 | 8.0 | Uterus Cancer 064011 | 17.8 | 14.5 |
| CC Gr. 2 ascend colon | 58.2 | 47.6 | Normal Thyroid | 8.7 | 5.0 |
| (ODO3921) | | | | | |
| CC Margin (ODO3921) | 16.3 | 8.2 | Thyroid Cancer 064010 | 10.2 | 8.9 |
| CC from Partial | 28.5 | 22.5 | Thyroid Cancer | 2.9 | 4.5 |
| Hepatectomy (ODO4309) | | | A302152 | | |
| Mets | | | | | |
| Liver Margin (ODO4309) | 18.4 | 8.1 | Thyroid Margin | 10.3 | 7.3 |
| | | | A302153 | | |
| Colon mets to lung | 12.0 | 15.7 | Normal Breast | 18.0 | 17.1 |
| (OD04451-01) | | | | | |
| Lung Margin (OD04451- | 6.5 | 3.9 | Breast Cancer | 15.7 | 11.5 |
| 02) | | | (OD04566) | | |
| Normal Prostate 6546-1 | 23.2 | 17.3 | Breast Cancer | 42.6 | 49.7 |
| | | | (OD04590-01) | | |
| Prostate Cancer (OD04410) | 29.5 | 12.6 | Breast Cancer Mets | 42.6 | 31.2 |
| | | | (OD04590-03) | | |
| Prostate Margin (OD04410) | 18.3 | 12.7 | Breast Cancer | 33.4 | 23.3 |
| | | | Metastasis (OD04655- | | |
| | | | 05) | | |
| Prostate Cancer (OD04720- | 39.0 | 28.9 | Breast Cancer 064006 | 15.6 | 15.3 |
| 01) | | | | | |
| Prostate Margin (OD04720- | 32.8 | 32.3 | Breast Cancer 1024 | 64.2 | 49.0 |
| 02) | | | | | |
| Normal Lung 061010 | 47.3 | 42.9 | Breast Cancer 9100266 | 12.1 | 12.2 |
| Lung Met to Muscle | 16.5 | 19.6 | Breast Margin 9100265 | 17.7 | 20.0 |
| (ODO4286) | | | | | |
| Muscle Margin (ODO4286) | 3.5 | 4.5 | Breast Cancer A209073 | 27.5 | 33.2 |
| Lung Malignant Cancer | 25.3 | 26.2 | Breast Margin | 22.8 | 27.7 |
| (OD03126) | | | A2090734 | | |
| Lung Margin (OD03126) | 9.5 | 12.3 | Normal Liver | 14.4 | 8.1 |
| Lung Cancer (OD04404) | 19.8 | 13.9 | Liver Cancer 064003 | 4.9 | 4.1 |
| Lung Margin (OD04404) | 12.3 | 10.1 | Liver Cancer 1025 | 9.3 | 8.3 |
| Lung Cancer (OD04565) | 4.8 | 3.8 | Liver Cancer 1026 | 8.4 | 8.8 |
| Lung Margin (OD04565) | 13.7 | 8.3 | Liver Cancer 6004-T | 10.2 | 11.9 |
| Lung Cancer (OD04237- | 11.0 | 9.9 | Liver Tissue 6004-N | 2.5 | 2.0 |
| 01) | | | | | |
| Lung Margin (OD04237- | 21.0 | 23.0 | Liver Cancer 6005-T | 7.3 | 6.2 |
| 02) | | | | | |
| Ocular Mel Met to Liver | 4.9 | 3.5 | Liver Tissue 6005-N | 2.9 | 3.2 |
| (ODO4310) | | | | | |
| Liver Margin (ODO4310) | 5.2 | 4.0 | Normal Bladder | 17.3 | 15.2 |
| Melanoma Mets to Lung | 1.3 | 3.5 | Bladder Cancer 1023 | 11.0 | 12.3 |
| (OD04321) | | | | | |
| Lung Margin (OD04321) | 18.2 | 23.7 | Bladder Cancer | 46.3 | 47.0 |
| | | | A302173 | | |
| Normal Kidney | 65.5 | 50.3 | Bladder Cancer | 65.1 | 42.3 |
| | | | (OD04718-01) | | |
| Kidney Ca, Nuclear grade 2 | 49.0 | 36.1 | Bladder Normal | 11.6 | 6.7 |
| (OD04338) | | | Adjacent (OD04718-03) | | |
| Kidney Margin (OD04338) | 48.3 | 33.2 | Normal Ovary | 2.9 | 2.3 |
| Kidney Ca Nuclear grade | 19.8 | 14.0 | Ovarian Cancer 064008 | 32.5 | 28.1 |
| 1/2 (OD04339) | | | | | |
| Kidney Margin (OD04339) | 81.8 | 65.1 | Ovarian Cancer | 9.3 | 8.0 |
| | | | (OD04768-07) | | |
| Kidney Ca, Clear cell type | 100.0 | 100.0 | Ovary Margin | 8.8 | 6.3 |
| (OD04340) | | | (OD04768-08) | | |
| Kidney Margin (OD04340) | 82.4 | 66.0 | Normal Stomach | 15.4 | 16.3 |
| Kidney Ca, Nuclear grade 3 | 9.9 | 9.9 | Gastric Cancer 9060358 | 10.4 | 4.0 |
| (OD04348) | | | | | |
| Kidney Margin (OD04348) | 45.1 | 42.6 | Stomach Margin | 15.6 | 20.6 |
| | | | 9060359 | | |
| Kidney Cancer (OD04622- | 24.3 | 14.2 | Gastric Cancer 9060395 | 25.9 | 26.4 |
| 01) | | | | | |
| Kidney Margin (OD04622- | 9.4 | 10.0 | Stomach Margin | 35.8 | 29.9 |
| 103) | | | 9060394 | | |
| Kidney Cancer (OD04450- | 6.5 | 6.2 | Gastric Cancer 9060397 | 79.0 | 64.2 |
| 01) | | | | | |
| Kidney Margin (OD04450- | 22.4 | 33.0 | Stomach Margin | 10.9 | 8.4 |
| 03) | | | 9060396 | | |
| Kidney Cancer 8120607 | 33.4 | 24.5 | Gastric Cancer 064005 | 23.8 | 27.5 |

TABLE 93
Panel 4D

| | Rel. | Rel. | Rel. | Rel. | Rel. | Rel. | Rel. |
|---|---|---|---|---|---|---|---|
| | Exp. (%) | Exp. (%) | Exp. (%) | Exp. (%) | Exp. (%) | Exp. (%) | Exp. (%) |
| | Ag1845, | Ag1845, | Ag1845, | Ag2589, | Ag2589, | Ag2621, | Ag2915, |
| | Run | Run | Run | Run | Run | Run | Run |
| Tissue Name | 148648440 | 149957765 | 162733767 | 164289988 | 164347841 | 164299478 | 164403111 |
| Secondary Th1 act | 1.8 | 2.4 | 1.8 | 1.7 | 1.7 | 2.1 | 2.0 |
| Secondary Th2 act | 1.3 | 1.5 | 1.5 | 2.4 | 2.4 | 2.0 | 1.7 |
| Secondary Tr1 act | 2.5 | 2.7 | 3.3 | 2.3 | 2.3 | 3.0 | 2.1 |
| Secondary Th1 | 0.6 | 0.4 | 0.5 | 0.4 | 0.4 | 0.5 | 0.6 |
| rest | | | | | | | |
| Secondary Th2 | 1.0 | 0.7 | 0.9 | 0.6 | 0.6 | 1.1 | 0.5 |
| rest | | | | | | | |
| Secondary Tr1 rest | 1.2 | 1.2 | 0.7 | 1.1 | 1.1 | 1.8 | 0.9 |
| Primary Th1 act | 3.1 | 1.9 | 1.2 | 2.0 | 2.0 | 3.1 | 2.0 |
| Primary Th2 act | 2.1 | 4.5 | 2.3 | 3.0 | 3.0 | 4.6 | 3.8 |
| Primary Tr1 act | 3.8 | 5.1 | 2.8 | 3.1 | 3.1 | 6.2 | 4.2 |
| Primary Th1 rest | 2.7 | 3.4 | 3.1 | 2.8 | 2.8 | 6.0 | 4.0 |
| Primary Th2 rest | 2.2 | 2.3 | 1.4 | 1.6 | 1.6 | 3.8 | 1.8 |
| Primary Tr1 rest | 1.4 | 2.3 | 1.9 | 2.0 | 2.0 | 2.6 | 2.4 |
| CD45RA CD4 | 1.0 | 1.4 | 1.0 | 1.8 | 1.8 | 1.7 | 1.7 |
| lymphocyte act | | | | | | | |
| CD45RO CD4 | 1.2 | 1.9 | 2.1 | 3.4 | 3.4 | 1.9 | 2.2 |
| lymphocyte act | | | | | | | |
| CD8 lymphocyte | 0.7 | 0.5 | 0.5 | 1.1 | 1.1 | 0.8 | 1.4 |
| act | | | | | | | |
| Secondary CD8 | 0.7 | 0.7 | 1.0 | 1.8 | 1.8 | 2.2 | 1.9 |
| lymphocyte rest | | | | | | | |
| Secondary CD8 | 0.6 | 1.2 | 0.8 | 1.3 | 1.3 | 0.8 | 1.9 |
| lymphocyte act | | | | | | | |
| CD4 lymphocyte | 1.0 | 1.5 | 0.7 | 1.2 | 1.2 | 1.6 | 1.2 |
| none | | | | | | | |
| 2ry | 0.6 | 1.0 | 1.0 | 1.9 | 1.9 | 1.9 | 1.1 |
| Th1/Th2/Tr1_anti- | | | | | | | |
| CD95 CH11 | | | | | | | |
| LAK cells rest | 4.3 | 4.4 | 5.6 | 12.2 | 12.2 | 8.5 | 6.7 |
| LAK cells IL-2 | 0.2 | 0.3 | 0.4 | 1.4 | 1.4 | 1.1 | 0.7 |
| LAK cells IL- | 1.1 | 0.6 | 1.9 | 1.7 | 1.7 | 2.3 | 1.2 |
| 2 + IL-12 | | | | | | | |
| LAK cells IL- | 1.5 | 2.8 | 1.7 | 2.7 | 2.7 | 3.1 | 3.0 |
| 2 + IFN gamma | | | | | | | |
| LAK cells IL- | 1.1 | 1.7 | 1.4 | 2.6 | 2.6 | 3.2 | 2.2 |
| 2 + IL-18 | | | | | | | |
| LAK cells | 2.3 | 2.3 | 2.1 | 4.1 | 4.1 | 3.6 | 3.9 |
| PMA/ionomycin | | | | | | | |
| NK Cells IL-2 rest | 0.1 | 0.4 | 0.3 | 0.6 | 0.6 | 0.8 | 0.6 |
| Two Way MLR 3 | 7.0 | 8.0 | 3.8 | 9.2 | 9.2 | 9.5 | 8.8 |
| day | | | | | | | |
| Two Way MLR 5 | 1.9 | 2.0 | 2.2 | 3.9 | 3.9 | 4.4 | 2.5 |
| day | | | | | | | |
| Two Way MLR 7 | 1.1 | 0.8 | 0.4 | 1.8 | 1.8 | 1.6 | 1.1 |
| day | | | | | | | |
| PBMC rest | 2.7 | 3.3 | 2.0 | 6.8 | 6.8 | 5.7 | 4.6 |
| PBMC PWM | 2.0 | 2.6 | 0.9 | 4.3 | 4.3 | 5.8 | 5.4 |
| PBMC PHA-L | 1.0 | 0.9 | 0.9 | 2.2 | 2.2 | 2.0 | 2.5 |
| Ramos (B cell) | 9.3 | 12.9 | 8.8 | 13.8 | 13.8 | 19.2 | 15.2 |
| none | | | | | | | |
| Ramos (B cell) | 14.8 | 17.6 | 15.9 | 22.7 | 22.7 | 30.6 | 26.2 |
| ionomycin | | | | | | | |
| B lymphocytes | 9.2 | 11.7 | 10.4 | 10.9 | 10.9 | 18.7 | 11.3 |
| PWM | | | | | | | |
| B lymphocytes | 15.7 | 16.5 | 16.4 | 14.6 | 14.6 | 26.8 | 20.2 |
| CD40L and IL-4 | | | | | | | |
| EOL-1 dbcAMP | 27.2 | 34.9 | 23.0 | 23.7 | 23.7 | 26.8 | 25.3 |
| EOL-1 dbcAMP | 100.0 | 100.0 | 100.0 | 100.0 | 100.0 | 100.0 | 100.0 |
| PMA/ionomycin | | | | | | | |
| Dendritic cells | 9.2 | 9.6 | 6.7 | 12.9 | 12.9 | 9.9 | 8.6 |
| none | | | | | | | |
| Dendritic cells | 13.9 | 16.5 | 13.9 | 19.2 | 19.2 | 23.3 | 17.3 |
| LPS | | | | | | | |
| Dendritic cells | 14.4 | 20.4 | 11.4 | 16.4 | 16.4 | 17.1 | 11.7 |
| anti-CD40 | | | | | | | |
| Monocytes rest | 3.4 | 3.8 | 2.3 | 3.6 | 3.6 | 4.5 | 4.0 |
| Monocytes LPS | 6.4 | 8.9 | 6.7 | 11.5 | 11.5 | 12.2 | 11.2 |
| Macrophages rest | 4.9 | 6.3 | 4.2 | 6.2 | 6.2 | 10.5 | 7.9 |
| Macrophages LPS | 7.3 | 9.2 | 7.3 | 12.0 | 12.0 | 15.7 | 13.6 |
| HUVEC none | 1.2 | 1.1 | 1.0 | 1.8 | 1.8 | 2.3 | 1.1 |
| HUVEC starved | 2.2 | 2.5 | 2.0 | 3.1 | 3.1 | 4.3 | 3.9 |
| HUVEC IL-1beta | 7.7 | 7.7 | 3.3 | 6.2 | 6.2 | 9.6 | 7.6 |
| HUVEC IFN | 1.9 | 1.3 | 0.9 | 2.4 | 2.4 | 1.8 | 2.0 |
| gamma | | | | | | | |
| HUVEC TNF | 14.7 | 15.0 | 14.6 | 22.1 | 22.1 | 26.1 | 20.7 |
| alpha + IFN | | | | | | | |
| gamma | | | | | | | |
| HUVEC TNF | 10.0 | 13.0 | 11.5 | 28.7 | 28.7 | 20.2 | 19.2 |
| alpha + IL4 | | | | | | | |
| HUVEC IL-11 | 0.8 | 2.0 | 1.1 | 1.8 | 1.8 | 1.1 | 1.3 |
| Lung | 0.6 | 1.2 | 1.5 | 2.0 | 2.0 | 2.8 | 2.2 |
| Microvascular EC | | | | | | | |
| none | | | | | | | |
| Lung | 27.2 | 37.6 | 28.5 | 54.3 | 54.3 | 56.6 | 48.3 |
| Microvascular EC | | | | | | | |
| TNFalpha + IL- | | | | | | | |
| 1beta | | | | | | | |
| Microvascular | 0.5 | 1.6 | 0.5 | 1.5 | 1.5 | 1.0 | 1.3 |
| Dermal EC none | | | | | | | |
| Microsvasular | 47.6 | 55.9 | 39.5 | 47.3 | 47.3 | 61.6 | 48.6 |
| Dermal EC | | | | | | | |
| TNFalpha + IL- | | | | | | | |
| 1beta | | | | | | | |
| Bronchial | 2.0 | 2.4 | 0.6 | 3.2 | 3.2 | 4.7 | 3.1 |
| epithelium | | | | | | | |
| TNFalpha + | | | | | | | |
| IL1beta | | | | | | | |
| Small airway | 1.2 | 0.6 | 0.5 | 0.4 | 0.4 | 0.9 | 0.8 |
| epithelium none | | | | | | | |
| Small airway | 2.1 | 4.2 | 3.4 | 3.7 | 3.7 | 5.4 | 5.6 |
| epithelium | | | | | | | |
| TNFalpha + IL- | | | | | | | |
| 1beta | | | | | | | |
| Coronery artery | 0.2 | 0.3 | 0.2 | 0.3 | 0.3 | 0.5 | 0.1 |
| SMC rest | | | | | | | |
| Coronery artery | 0.5 | 0.6 | 0.5 | 0.8 | 0.8 | 0.8 | 1.0 |
| SMC TNFalpha + | | | | | | | |
| IL-1beta | | | | | | | |
| Astrocytes rest | 0.4 | 1.0 | 0.6 | 0.4 | 0.4 | 0.8 | 0.8 |
| Astrocytes | 22.8 | 28.5 | 23.5 | 26.2 | 26.2 | 27.9 | 22.8 |
| TNFalpha + IL- | | | | | | | |
| 1beta | | | | | | | |
| KU-812 | 0.5 | 1.2 | 0.7 | 0.7 | 0.7 | 0.4 | 0.3 |
| (Basophil) rest | | | | | | | |
| KU-812 | 2.1 | 2.0 | 1.7 | 1.9 | 1.9 | 2.4 | 1.9 |
| (Basophil) | | | | | | | |
| PMA/ionomycin | | | | | | | |
| CCD1106 | 0.5 | 0.9 | 0.8 | 1.0 | 1.0 | 1.4 | 1.0 |
| (Keratinocytes) | | | | | | | |
| none | | | | | | | |
| CCD1106 | 1.3 | 1.0 | 0.6 | 2.8 | 2.8 | 3.7 | 2.3 |
| (Keratinocytes) | | | | | | | |
| TNFalpha + IL- | | | | | | | |
| 1beta | | | | | | | |
| Liver cirrhosis | 0.9 | 1.3 | 0.6 | 1.0 | 1.0 | 0.9 | 0.9 |
| Lupus kidney | 0.9 | 1.0 | 1.8 | 1.9 | 1.9 | 1.9 | 1.7 |
| NCI-H292 none | 4.2 | 4.1 | 2.6 | 2.6 | 2.6 | 3.3 | 3.2 |
| NCI-H292 IL-4 | 2.5 | 2.3 | 1.0 | 2.2 | 2.2 | 2.1 | 2.6 |
| NCI-H292 IL-9 | 2.5 | 3.7 | 3.2 | 3.2 | 3.2 | 4.7 | 2.8 |
| NCI-H292 IL-13 | 0.7 | 2.2 | 1.9 | 2.2 | 2.2 | 1.6 | 1.2 |
| NCI-H292 IFN | 5.5 | 4.4 | 3.6 | 5.0 | 5.0 | 4.5 | 4.2 |
| gamma | | | | | | | |
| HPAEC none | 0.5 | 0.6 | 0.5 | 1.5 | 1.5 | 0.8 | 0.9 |
| HPAEC TNF | 46.3 | 58.6 | 29.3 | 69.3 | 69.3 | 89.5 | 70.2 |
| alpha + IL-1 beta | | | | | | | |
| Lung fibroblast | 0.1 | 0.0 | 0.1 | 0.1 | 0.1 | 0.0 | 0.1 |
| none | | | | | | | |
| Lung fibroblast | 0.1 | 0.4 | 0.1 | 0.6 | 0.6 | 0.5 | 0.5 |
| TNF alpha + IL-1 | | | | | | | |
| beta | | | | | | | |
| Lung fibroblast | 0.0 | 0.1 | 0.1 | 0.1 | 0.1 | 0.1 | 0.0 |
| IL-4 | | | | | | | |
| Lung fibroblast | 0.0 | 0.0 | 0.0 | 0.0 | 0.0 | 0.1 | 0.0 |
| IL-9 | | | | | | | |
| Lung fibroblast | 0.0 | 0.0 | 0.0 | 0.0 | 0.0 | 0.0 | 0.0 |
| IL-13 | | | | | | | |
| Lung fibroblast | 0.0 | 0.0 | 0.0 | 0.3 | 0.3 | 0.1 | 0.2 |
| IFN gamma | | | | | | | |
| Dermal fibroblast | 0.1 | 0.4 | 0.0 | 0.7 | 0.7 | 0.6 | 0.4 |
| CCD1070 rest | | | | | | | |
| Dermal fibroblast | 2.2 | 2.2 | 2.0 | 3.2 | 3.2 | 4.2 | 2.9 |
| CCD1070 TNF | | | | | | | |
| alpha | | | | | | | |
| Dermal fibroblast | 0.1 | 0.5 | 0.5 | 0.7 | 0.7 | 0.4 | 0.5 |
| CCD1070 IL-1 | | | | | | | |
| beta | | | | | | | |
| Dermal fibroblast | 0.1 | 0.1 | 0.0 | 0.3 | 0.3 | 0.5 | 0.4 |
| IFN gamma | | | | | | | |
| Dermal fibroblast | 0.3 | 0.1 | 0.0 | 0.6 | 0.6 | 0.4 | 0.4 |
| IL-4 | | | | | | | |
| IBD Colitis 2 | 0.3 | 0.7 | 1.1 | 1.0 | 1.0 | 1.4 | 1.3 |
| IBD Crohn's | 0.2 | 0.3 | 0.2 | 0.4 | 0.4 | 0.5 | 0.3 |
| Colon | 2.2 | 2.7 | 3.1 | 5.8 | 5.8 | 9.6 | 5.3 |
| Lung | 1.9 | 2.8 | 4.4 | 3.2 | 3.2 | 2.2 | 4.5 |
| Thymus | 8.6 | 9.3 | 7.4 | 11.0 | 11.0 | 14.2 | 12.9 |
| Kidney | 3.6 | 5.2 | 3.8 | 4.5 | 4.5 | 7.1 | 7.0 |

AI_comprehensive panel_v1.0 Summary: Ag1845 The NOV14 transcript is expressed at low levels in many different disease tissues. In comparison, normal lung and joint tissues express none or extremely low levels of this transcript. Since the NOV14 transcript is expressed in monocytes, and matched control tissues most likely contain these inflammatory cells (psoriasis, Crohn's and ulcerative colitis) it is not surprising that transcript expression is detected at these sites. The NOV14 transcript encodes B7-H2, which has been shown to be important in antigen presentation. It is a ligand for ICOS and serves as a costimulatory molecule (see panel 4). Therefore, therapeutics designed with the NOV14 transcript could reduce or inhibit antigen presentation and be important in the treatment of diseases such as asthma, IBD, psoriasis and arthritis in which T cells are chronically stimulated.

CNS_neurodegeneration_v1.0 Summary: Ag1845/Ag2589/Ag2621 /Ag2915 Multiple experiments with two different probe and primer sets are in excellent agreement. In all cases, the expression of the NOV14 gene is up-regulated in the temporal cortex of Alzheimer's disease patients when compared to non-demented controls. This difference is apparent when data are analyzed via ANCOVA, using overall RNA quality and/or quantity as a covariate. The up-regulation of the NOV14 gene is most apparent in the variant detected by Ag1845. The temporal cortex is a region that shows degeneration at the mid-stages of this disease. Thus, it is likely that the phenomenon of neurodegeneration was captured in this region, as opposed to the hippocampus and entorhinal cortex where a large number of neurons are already lost by the time of death in AD. Furthermore, in the occipital cortex (where neurodegeneration does not occur in Alzheimer's) the NOV14 gene is not found to be up-regulated in the same patients. Taken together, these data suggest that this gene is at least a marker of Alzheimer's-like neurodegeneration, and is probably involved in the process of neurodegeneration.

Furthermore, the NOV14 gene is a form of B7 protein (B7-H2B), which plays a role in inflammation. Neuroinflammation has been implicated in AD, to the extent that long-term usage of anti-inflammatory agents has been correlated with a reduced incidence of Alzheimer's in retrospective studies. This gene therefore represents an excellent drug target for the treatment of Alzheimer's disease, and any other neuroinflammatory condition.

Panel 1 Summary: Ag210 The expression of the NOV14 gene appears to be highest in a sample derived from normal brain tissue of the cerebellum (CT=19.5). Thus, the expression of this gene could be used to distinguish normal cerebellum tissue from the other tissues in the panel.

The NOV14 gene also shows widespread and high-to-moderate expression in metabolic tissues including pancreas, adrenal. Although a role for B7-H2 molecules in metabolism or endocrinology has not been described, based on its expression this gene product may be an antibody target for the treatment of metabolic or endocrine disease, including obesity and Types 1 and 2 diabetes.

Panel 1.3D Summary: Ag1845/Ag2589/Ag2621/Ag2915 Multiple experiments with two different probe and primer sets are in excellent agreement. Highest expression of the NOV14 gene is seen in the brain, fetal kidney, and a breast cancer cell line.

Expression in the CNS panel confirms the expression of the NOV14 gene in the CNS. Please see panel CNS_Neurodegeneration for a discussion of utility of this gene in the central nervous system.

Higher levels of expression are also consistently seen in fetal skeletal muscle (CTs=29-30), when compared to expression in adult skeletal muscle (CTs=33-35). Thus, expression of the NOV14 gene could be used to differentiate between the adult and fetal sources of this tissue.

The NOV14 gene product is also moderately expressed in pancreas, adrenal, thyroid, pituitary, adult and fetal liver, adult and fetal heart, and adipose. Based on its expression profile in metabolic tissues, the NOV14 gene product may be useful in the diagnosis and/or treatment of metabolic disease, including obesity and diabetes.

Panel 2.2 Summary: Ag2621 The expression of the NOV14 gene appears to be highest in a sample derived from a normal kidney margin (CT=29.1). In addition, there appears to be substantial expression associated with several kidney cancer samples. Thus, the expression of the NOV14 gene could be used to distinguish this normal kidney sample from others in the panel. Moreover, therapeutic modulation of this gene, through the use of small molecule drugs, protein therapeutics or antibodies might be beneficial for the treatment of kidney cancer.

Panel 2D Summary: Ag1845 The expression of the NOV14 gene was assessed in two independent runs in panel 2D with excellent concordance between runs. The expression of this gene is highest in a sample derived from a kidney cancer (CTs=28). In addition, there is substantial expression associated with other samples derived from kidney tissue, bladder cancer and breast cancer. Thus, the expression of the NOV14 gene could be used to distinguish this kidney cancer sample from other samples in the panel. Moreover, therapeutic modulation of this gene, through the use of small molecule drugs, protein therapeutics or antibodies might be beneficial for the treatment of kidney cancer, breast cancer or bladder cancer.

Panel 4D Summary: Ag2589/Ag2621/Ag2915/Ag1845 The NOV14 transcript is highly expressed in activated EOL cells, activated lung and dermal microvascular endothelium, activated human pulmonary aortic endothelial cells and in TNFalpha activated human umbilical vein endothelial cells. CG55790-02 encodes B7-H2, which has been shown to be important in antigen presentation. It is a ligand for ICOS and serves as a costimulatory molecule (Ref. 1-2). Therefore, monoclonal antibody therapeutics designed with the CG55790-02 protein product may reduce or inhibit antigen presentation and be important in the treatment of diseases such as asthma in which T cells are chronically stimulated.

References:

Ling V, Wu P W, Finnerty H F, Bean K M, Spaulding V, Fouser L A, Leonard J P, Hunter S E, Zollner R, Thomas J L, Miyashiro J S, Jacobs K A, Collins M. Cutting edge: identification of GL50, a novel B7-like protein that functionally binds to ICOS receptor. J Immunol Feb. 15, 2000;164(4):1653-7

By the genetic selection of mouse cDNAs encoding secreted proteins, a B7-like cDNA clone termed mouse GL50 (mGL50) was isolated encoding a 322-aa polypeptide identical with B7h. Isolation of the human ortholog of this cDNA (hGL50) revealed a coding sequence of 309 aa residues with 42% sequence identity with mGL50. Northern analysis indicated GL50 to be present in many tissues including lymphoid, embryonic yolk sac, and fetal liver samples. Of the CD28, CTLA4, and ICOS fusion constructs tested, flow cytometric analysis demonstrated only mouse ICOS-IgG binding to mGL50 cell transfectants. Subsequent phenotyping demonstrated high levels of ICOS ligand staining on splenic CDl9+B cells and low levels on CD3+T cells. These results indicate that GL50 is a specific ligand for the ICOS receptor and suggest that the GL50-ICOS interaction functions in lymphocyte costimulation.

Wang S, Zhu G, Chapoval A I, Dong H, Tamada K, Ni J, Chen L. Costimulation of T cells by B7-H2, a B7-like molecule that binds ICOS. Blood Oct. 15, 2000;96(8):2808-13

This report describes a new human B7-like gene designated B7-H2. Cell surface expression of B7-H2 protein is detected in monocyte-derived immature dendritic cells. Soluble B7-H2 and immunoglobulin (Ig) fusion protein, B7-H2Ig, binds activated but not resting T cells and the binding is abrogated by inducible costimulator Ig (ICOSIg), but not CTLA4Ig. In addition, ICOSIg stains Chinese hamster ovary cells transfected with B7-H2 gene. By suboptimal cross-linking of CD3, costimulation of T-cell proliferation by B7-H2Ig is dose-dependent and correlates with secretion of interleukin (IL)-2, whereas optimal CD3 ligation preferentially stimulates IL-10 production. The results indicate that B7-H2 is a putative ligand for the ICOS T-cell molecule. (Blood. 2000;96:2808-2813) PMID: 11023515

N. NOV15: Galactosyl Transferase

Expression of the NOV15 gene (CG56252-01) was assessed using the primer-probe set Ag2902, described in Table 94. Results of the RTQ-PCR runs are shown in Tables 95, 96 and 97.

TABLE 94
Probe Name Ag2902

| Primers | Sequences | Length | Start Position | SEQ ID NO: |
|---|---|---|---|---|
| Forward | 5′-acgctcaaggagatccactt-3′ | 20 | 682 | 261 |
| Probe | TET-5′-tctagcctgggcctcagctttctg-3′-TAMRA | 24 | 702 | 262 |
| Reverse | 5′-cacgttcacgaacacatctg-3′ | 20 | 758 | 263 |

TABLE 95
Panel 1.3D

| | Rel. Exp. (%) | | Rel. Exp. (%) |
|---|---|---|---|
| | Ag2902, Run | | Ag2902, Run |
| Tissue Name | 160999861 | Tissue Name | 160999861 |
| Liver adenocarcinoma | 18.7 | Kidney (fetal) | 6.8 |
| Pancreas | 1.5 | Renal ca. 786-0 | 3.7 |
| Pancreatic ca. CAPAN2 | 0.2 | Renal ca. A498 | 17.2 |
| Adrenal gland | 4.0 | Renal ca. RXF 393 | 8.8 |
| Thyroid | 29.5 | Renal ca. ACHN | 3.3 |
| Salivary gland | 3.1 | Renal ca. UO-31 | 5.9 |
| Pituitary gland | 15.7 | Renal ca. TK-10 | 0.8 |
| Brain (fetal) | 0.2 | Liver | 0.7 |
| Brain (whole) | 8.5 | Liver (fetal) | 2.5 |
| Brain (amygdala) | 4.2 | Liver ca. (hepatoblast) HepG2 | 0.6 |
| Brain (cerebellum) | 1.8 | Lung | 6.8 |
| Brain (hippocampus) | 15.0 | Lung (fetal) | 9.5 |
| Brain (substantia nigra) | 1.6 | Lung ca. (small cell) LX-1 | 1.4 |
| Brain (thalamus) | 5.8 | Lung ca. (small cell) NCI-H69 | 2.2 |
| Cerebral Cortex | 40.3 | Lung ca. (s. cell var.) SHP-77 | 2.1 |
| Spinal cord | 27.7 | Lung ca. (large cell) NCI-H460 | 3.7 |
| glio/astro U87-MG | 86.5 | Lung ca. (non-sm. cell) A549 | 4.9 |
| glio/astro U-118-MG | 12.2 | Lung ca. (non-s. cell) NCI-H23 | 12.9 |
| astrocytoma SW1783 | 40.3 | Lung ca. (non-s. cell) HOP-62 | 13.8 |
| neuro*; met SK-N-AS | 4.8 | Lung ca. (non-s. cl) NCI-H522 | 6.9 |
| astrocytoma SF-539 | 26.8 | Lung ca. (squam.) SW 900 | 3.6 |
| astrocytoma SNB-75 | 22.4 | Lung ca. (squam.) NCI-H596 | 0.3 |
| glioma SNB-19 | 22.7 | Mammary gland | 7.2 |
| glioma U251 | 7.5 | Breast ca.* (pl. ef) MCF-7 | 6.6 |
| glioma SF-295 | 24.3 | Breast ca.* (pl. ef) MDA-MB- | 5.7 |
| | | 231 | |
| Heart (fetal) | 35.6 | Breast ca.* (pl. ef) T47D | 1.8 |
| Heart | 9.0 | Breast ca. BT-549 | 8.5 |
| Skeletal muscle (fetal) | 60.7 | Breast ca. MDA-N | 1.3 |
| Skeletal muscle | 2.4 | Ovary | 100.0 |
| Bone marrow | 0.6 | Ovarian ca. OVCAR-3 | 3.6 |
| Thymus | 9.5 | Ovarian ca. OVCAR-4 | 1.0 |
| Spleen | 7.6 | Ovarian ca. OVCAR-5 | 5.5 |
| Lymph node | 2.3 | Ovarian ca. OVCAR-8 | 9.7 |
| Colorectal | 7.7 | Ovarian ca. IGROV-1 | 6.2 |
| Stomach | 5.3 | Ovarian ca.* (ascites) SK-OV- | 2.0 |
| | | 3 | |
| Small intestine | 9.7 | Uterus | 9.7 |
| Colon ca. SW480 | 2.9 | Placenta | 4.4 |
| Colon ca.* SW620 (SW480 met) | 3.6 | Prostate | 8.1 |
| Colon ca. HT29 | 5.5 | Prostate ca.* (bone met)PC-3 | 1.9 |
| Colon ca. HCT-116 | 0.7 | Testis | 14.9 |
| Colon ca. CaCo-2 | 2.0 | Melanoma Hs688(A).T | 17.1 |
| Colon ca. tissue (ODO3866) | 8.9 | Melanoma* (met) Hs688(B).T | 17.6 |
| Colon ca. HCC-2998 | 14.5 | Melanoma UACC-62 | 1.1 |
| Gastric ca.* (liver met) NCI-N87 | 10.4 | Melanoma M14 | 0.6 |
| Bladder | 7.0 | Melanoma LOX IMVI | 0.0 |
| Trachea | 25.0 | Melanoma* (met) SK-MEL-5 | 0.1 |
| Kidney | 9.9 | Adipose | 5.8 |

TABLE 96
Panel 2D

| | Rel. Exp. (%) | | Rel. Exp. (%) |
|---|---|---|---|
| | Ag2902, Run | | Ag2902, Run |
| Tissue Name | 160997627 | Tissue Name | 160997627 |
| Normal Colon | 21.2 | Kidney Margin 8120608 | 11.3 |
| CC Well to Mod Diff (ODO3866) | 6.1 | Kidney Cancer 8120613 | 1.4 |
| CC Margin (ODO3868) | 4.4 | Kidney Margin 8120614 | 23.0 |
| CC Gr. 2 rectosigmoid (ODO3868) | 3.3 | Kidney Cancer 9010320 | 35.6 |
| CC Margin (ODO3868) | 1.5 | Kidney Margin 9010321 | 17.0 |
| CC Mod Diff (ODO3920) | 9.3 | Normal Uterus | 10.5 |
| CC Margin (ODO3920) | 4.6 | Uterus Cancer 064011 | 11.4 |
| CC Gr. 2 ascend colon (ODO3921) | 11.3 | Normal Thyroid | 51.8 |
| CC Margin (ODO3921) | 5.6 | Thyroid Cancer 064010 | 46.0 |
| CC from Partial Hepatectomy | 6.7 | Thyroid Cancer A302152 | 18.0 |
| (ODO4309) Mets | | | |
| Liver Margin (ODO4309) | 3.9 | Thyroid Margin A302153 | 33.9 |
| Colon mets to lung (OD04451-01) | 6.1 | Normal Breast | 20.4 |
| Lung Margin (OD04451-02) | 7.3 | Breast Cancer (OD04566) | 6.5 |
| Normal Prostate 6546-1 | 9.9 | Breast Cancer (OD04590-01) | 100.0 |
| Prostate Cancer (OD04410) | 12.3 | Breast Cancer Mets (OD04590- | 97.9 |
| | | 03) | |
| Prostate Margin (OD04410) | 26.8 | Breast Cancer Metastasis | 24.0 |
| | | (OD04655-05) | |
| Prostate Cancer (OD04720-01) | 19.6 | Breast Cancer 064006 | 10.3 |
| Prostate Margin (OD04720-02) | 38.2 | Breast Cancer 1024 | 36.9 |
| Normal Lung 061010 | 20.0 | Breast Cancer 9100266 | 23.8 |
| Lung Met to Muscle (ODO4286) | 19.1 | Breast Margin 9100265 | 18.2 |
| Muscle Margin (ODO4286) | 7.9 | Breast Cancer A209073 | 21.5 |
| Lung Malignant Cancer (OD03126) | 20.7 | Breast Margin A2090734 | 12.4 |
| Lung Margin (OD03126) | 17.2 | Normal Liver | 2.4 |
| Lung Cancer (OD04404) | 19.5 | Liver Cancer 064003 | 0.9 |
| Lung Margin (OD04404) | 21.9 | Liver Cancer 1025 | 2.9 |
| Lung Cancer (OD04565) | 17.6 | Liver Cancer 1026 | 5.0 |
| Lung Margin (OD04565) | 9.8 | Liver Cancer 6004-T | 2.3 |
| Lung Cancer (OD04237-01) | 13.2 | Liver Tissue 6004-N | 3.4 |
| Lung Margin (OD04237-02) | 16.2 | Liver Cancer 6005-T | 6.7 |
| Ocular Mel Met to Liver | 2.6 | Liver Tissue 6005-N | 4.5 |
| (ODO4310) | | | |
| Liver Margin (ODO4310) | 4.9 | Normal Bladder | 13.1 |
| Melanoma Mets to Lung | 9.8 | Bladder Cancer 1023 | 8.6 |
| (OD04321) | | | |
| Lung Margin (OD04321) | 28.3 | Bladder Cancer A302173 | 17.1 |
| Normal Kidney | 28.9 | Bladder Cancer (OD04718-01) | 35.8 |
| Kidney Ca, Nuclear grade 2 | 29.3 | Bladder Normal Adjacent | 17.7 |
| (OD04338) | | (OD04718-03) | |
| Kidney Margin (OD04338) | 22.7 | Normal Ovary | 22.5 |
| Kidney Ca Nuclear grade 1/2 | 9.3 | Ovarian Cancer 064008 | 34.9 |
| (OD04339) | | | |
| Kidney Margin (OD04339) | 21.2 | Ovarian Cancer (OD04768-07) | 15.8 |
| Kidney Ca, Clear cell type | 66.4 | Ovary Margin (OD04768-08) | 10.6 |
| (OD04340) | | | |
| Kidney Margin (OD04340) | 23.5 | Normal Stomach | 15.2 |
| Kidney Ca, Nuclear grade 3 | 31.9 | Gastric Cancer 9060358 | 3.2 |
| (OD04348) | | | |
| Kidney Margin (OD04348) | 17.2 | Stomach Margin 9060359 | 10.8 |
| Kidney Cancer (OD04622-01) | 84.1 | Gastric Cancer 9060395 | 12.3 |
| Kidney Margin (OD04622-03) | 3.5 | Stomach Margin 9060394 | 15.4 |
| Kidney Cancer (OD04450-01) | 10.5 | Gastric Cancer 9060397 | 22.1 |
| Kidney Margin (OD04450-03) | 2.3 | Stomach Margin 9060396 | 7.2 |
| Kidney Cancer 8120607 | 7.2 | Gastric Cancer 064005 | 11.3 |

TABLE 97
Panel 4D

| | Rel. Exp. (%) | | Rel. Exp. (%) |
|---|---|---|---|
| | Ag2902, Run | | Ag2902, Run |
| Tissue Name | 159633371 | Tissue Name | 159633371 |
| Secondary Th1 act | 13.9 | HUVEC IL-1beta | 0.8 |
| Secondary Th2 act | 17.0 | HUVEC IFN gamma | 5.4 |
| Secondary Tr1 act | 27.7 | HUVEC TNF alpha + IFN gamma | 2.2 |
| Secondary Th1 rest | 11.7 | HUVEC TNF alpha + IL4 | 1.5 |
| Secondary Th2 rest | 10.6 | HUVEC IL-1 1 | 3.8 |
| Secondary Tr1 rest | 14.1 | Lung Microvascular EC none | 8.0 |
| Primary Th1 act | 6.0 | Lung Microvascular EC TNFalpha | 3.1 |
| | | + IL-1beta | |
| Primary Th2 act | 5.1 | Microvascular Dermal EC none | 5.5 |
| Primary Tr1 act | 8.4 | Microsvasular Dermal EC | 1.4 |
| | | TNFalpha + IL-1beta | |
| Primary Th1 rest | 28.1 | Bronchial epithelium TNFalpha + | 1.5 |
| | | IL1beta | |
| Primary Th2 rest | 12.5 | Small airway epithelium none | 8.4 |
| Primary Tr1 rest | 12.4 | Small airway epithelium TNFalpha | 6.8 |
| | | + IL-1beta | |
| CD45RA CD4 lymphocyte act | 18.9 | Coronery artery SMC rest | 30.4 |
| CD45RO CD4 lymphocyte act | 4.9 | Coronery artery SMC TNFalpha + | 28.1 |
| | | IL-1beta | |
| CD8 lymphocyte act | 4.0 | Astrocytes rest | 4.3 |
| Secondary CD8 lymphocyte rest | 8.7 | Astrocytes TNFalpha + IL-1beta | 3.2 |
| Secondary CD8 lymphocyte act | 6.3 | KU-812 (Basophil) rest | 3.3 |
| CD4 lymphocyte none | 4.2 | KU-812 (Basophil) | 6.9 |
| | | PMA/ionomycin | |
| 2ry Th1/Th2/Tr1_anti-CD95 CH11 | 8.0 | CCD1106 (Keratinocytes) none | 20.0 |
| LAK cells rest | 6.1 | CCD1106 (Keratinocytes) | 5.2 |
| | | TNFalpha + IL-1 beta | |
| LAK cells IL-2 | 5.8 | Liver cirrhosis | 6.2 |
| LAK cells IL-2 + IL-12 | 6.9 | Lupus kidney | 4.0 |
| LAK cells IL-2 + IFN gamma | 7.7 | NCI-H292 none | 30.6 |
| LAK cells IL-2 + IL-18 | 9.9 | NCI-H292 IL-4 | 39.0 |
| LAK cells PMA/ionomycin | 1.2 | NCI-H292 IL-9 | 34.4 |
| NK Cells IL-2 rest | 3.1 | NCI-H292 IL-13 | 29.5 |
| Two Way MLR 3 day | 4.0 | NCI-H292 IFN gamma | 35.8 |
| Two Way MLR 5 day | 2.5 | HPAEC none | 15.4 |
| Two Way MLR 7 day | 5.4 | HPAEC TNF alpha + IL-1 beta | 6.1 |
| PBMC rest | 2.8 | Lung fibroblast none | 49.0 |
| PBMC PWM | 14.7 | Lung fibroblast TNF alpha + IL-1 | 51.4 |
| | | beta | |
| PBMC PHA-L | 13.1 | Lung fibroblast IL-4 | 58.6 |
| Ramos (B cell) none | 1.4 | Lung fibroblast IL-9 | 46.7 |
| Ramos (B cell) ionomycin | 3.1 | Lung fibroblast IL-13 | 45.4 |
| B lymphocytes PWM | 8.5 | Lung fibroblast IFN gamma | 67.4 |
| B lymphocytes CD40L and IL-4 | 9.1 | Dermal fibroblast CCD1070 rest | 70.2 |
| EOL-1 dbcAMP | 0.9 | Dermal fibroblast CCD1070 TNF | 67.4 |
| | | alpha | |
| EOL-1 dbcAMP PMA/ionomycin | 3.7 | Dermal fibroblast CCD1070 IL-1 | 46.7 |
| | | beta | |
| Dendritic cells none | 2.6 | Dermal fibroblast IFN gamma | 48.3 |
| Dendritic cells LPS | 2.2 | Dermal fibroblast IL-4 | 100.0 |
| Dendritic cells anti-CD40 | 2.1 | IBD Colitis 2 | 0.1 |
| Monocytes rest | 2.2 | IBD Crohn's | 1.0 |
| Monocytes LPS | 3.7 | Colon | 18.7 |
| Macrophages rest | 3.4 | Lung | 39.5 |
| Macrophages LPS | 2.0 | Thymus | 16.7 |
| HUVEC none | 3.8 | Kidney | 7.7 |
| HUVEC starved | 4.1 | | |

CNS_neurodegeneration_v1.0 Summary: Ag2902 Results from one experiment with the NOV15 gene are not included. The amp plot indicates that there were experimental difficulties with this run.

Panel 1.3D Summary: Ag2902 Highest expression of the NOV15 gene is seen in the ovary (CT=28). Thus, expression of this gene could be used as a marker of normal ovarian tissue. The NOV15 gene also has moderate to high levels of expression in several endocrine/metabolic related tissues including, adipose, adrenal, GI tract, pituitary, skeletal muscle and thyroid. Therefore, a therapeutic modulator targeting the NOV15 gene and/or gene product may be useful in treating any number of diseases which afflict these tissues.

Significant expression is also detected in fetal skeletal muscle (CT=29). Interestingly, this gene is expressed at much higher levels in fetal when compared to adult skeletal muscle (CT=33.7). This observation suggests that expression of the NOV15 gene can be used to distinguish fetal from adult skeletal muscle. In addition, the relative overexpression of this gene in fetal skeletal muscle suggests that the protein product may enhance muscular growth or development in the fetus and thus may also act in a regenerative capacity in the adult. Therefore, therapeutic modulation of the protein encoded by this gene could be useful in treatment of muscle related diseases. More specifically, treatment of weak or dystrophic muscle with the protein encoded by the NOV15 gene could restore muscle mass or function.

There also appears to be substantial expression associated with various brain cancer cell lines. Moreover, therapeutic modulation of the NOV15 gene, through the use of small molecule drugs, protein therapeutics or antibodies might be beneficial in the treatment of brain cancer.

In addition, the NOV15 gene, a galactosyl transferase homolog, is expressed at low to moderate levels in all regions of the CNS examined. Galactosyl transferase plays a role in axonal myelination. Therefore, therapeutic modulation of this gene or its protein product may be of benefit in the treatment of multiple sclerosis or any demyelinating disease.

References:

Simons M, Kramer E M, Thiele C, Stoffel W, Trotter J. Assembly of myelin by association of proteolipid protein with cholesterol- and galactosylceramide-rich membrane domains. J Cell Biol Oct. 2, 2000;151(1):143-54

Myelin is a specialized membrane enriched in glycosphingolipids and cholesterol that contains a limited spectrum of proteins. We investigated the assembly of myelin components by oligodendrocytes and analyzed the role of lipid-protein interactions in this process. Proteolipid protein (PLP), the major myelin protein, was recovered from cultured oligodendrocytes from a low-density CHAPS-insoluble membrane fraction (CIMF) enriched in myelin lipids. PLP associated with the CIMF after leaving the endoplasmic reticulum but before exiting the Golgi apparatus, suggesting that myelin lipid and protein components assemble in the Golgi complex. The specific association of PLP with myelin lipids in CIMF was supported by the finding that it was efficiently cross-linked to photoactivable cholesterol, but not to phosphatidylcholine, which is underrepresented in both myelin and CIMF. Furthermore, depletion of cholesterol or inhibition of sphingolipid synthesis in oligodendrocytes abolished the association of PLP with CIMF. Thus, PLP may be recruited to myelin rafts, represented by CIMF, via lipid-protein interactions. In contrast to oligodendrocytes, after transfection in BHK cells, PLP is absent from isolated CIMF, suggesting that PLP requires specific lipids for raft association. In mice deficient in the enzyme ceramide galactosyl transferase, which cannot synthesize the main myelin glycosphingolipids, a large fraction of PLP no longer associates with rafts. Formation of a cholesterol- and galactosylceramide-rich membrane domain (myelin rafts) may be critical for the sorting of PLP and assembly of myelin in oligodendrocytes.

Panel 2D Summary: Ag2902 The expression of the NOV15 gene appears to be highest in a sample derived from a breast cancer (CT=28.1). There also appears to be substantial expression associated with other breast cancers, kidney cancer, bladder cancer and ovarian cancer. Thus, the expression of this gene could be used to distinguish this breast cancer sample from the rest of the samples on the panel. Moreover, therapeutic modulation of the NOV15 gene, through the use of small molecule drugs, protein therapeutics or antibodies might be beneficial in the treatment of breast cancer, ovarian cancer, bladder cancer or kidney cancer.

Panel 4D Summary: Ag2902 The NOV15 transcript is highly expressed in fibroblast and mucoepidermoid cell lines, with much lower expression in hematopoietic cell lines. The transcript encodes a galctosyl transferase isoform. This enzyme may be important both for the synthesis of galactose beta-1,4-N-acetylglucosamine and as a component of plasma membrane where it may function in intercellular recognition and/or adhesion (OMIM 137060). Protein glycosylation or trafficking through intracellular compartments in fibroblasts and leukocytes may be altered by the activity of this enzyme. This in turn could regulate the ability of these cells to express proteins involved in normal homeostasis in intercellular interactions. Therefore, therapeutics designed with the protein encoded by the NOV15 transcript could reduce or inhibit inflammation resulting from asthma, emphysema, psoriasis, IBD, and arthritis.

O. NOV16: Lymphocyte Antigen Precursor-Like Protein

Expression of the NOV16 gene (CG56303-01) was assessed using the primer-probe sets Ag3798 and Ag4119, described in Tables 98 and 99.

TABLE 98
Probe Name Ag3798

| Primers | Sequences | Length | Start Position | SEQ ID NO: |
|---|---|---|---|---|
| Forward | 5′-aacggagacaactgcttcaa-3′ | 20 | 115 | 264 |
| Probe | TET-5′-gctatggttgcctactgcatgaccac-3′-TAMRA | 26 | 151 | 265 |
| Reverse | 5′-taagttctctcccgcgaagt-3′ | 20 | 192 | 266 |

TABLE 99
Probe Name Ag4119

| Primers | Sequences | Length | Start Position | SEQ ID NO: |
|---|---|---|---|---|
| Forward | 5′-agatgaggacagcattgctg-3′ | 20 | 29 | 267 |
| Probe | TET-5′-cttgcagccctggctgtggctac-3′-TAMRA | 23 | 52 | 268 |
| Reverse | 5′-cagttgtctccgttgtaggc-3′ | 20 | 109 | 269 |

General_screening_panel_v1.4 Summary: Ag3798 Expression of the NOV16 gene is low/undetectable in all samples on this panel (CTs>35). (Data not shown.)

Panel 4.1D Summary: Ag3798/Ag4119 Expression of the NOV16 gene is low/undetectable in all samples on this panel (CTs>35). (Data not shown.)

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56;
(b) a variant of a mature form of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56; and
(d) a variant of an amino acid sequence selected from the group consisting of SEQ ID NOS: 2, 4, 6, 8, 10, 12, 14, 16, 18, 20, 22, 24, 26, 28, 30, 32, 34, 36, 38,40, 42, 44, 46, 48, 50, 52, 54 and 56 wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56;
(b) a variant of a mature form of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4,6,8, 10, 12, 14, 16, 18, 20, 22, 24, 26, 28, 30, 32, 34, 36, 38, 40, 42, 44, 46, 48, 50, 52, 54 and 56;
(d) a variant of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54 and 56, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55.

9. The nucleic acid molecule of claim 5, wherein said nucleic acid molecule comprises a nucleotide sequence selected from the group consisting of
(a) a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55;
(b) a nucleotide sequence differing by one or more nucleotides from a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53 and 55, or a complement of said nucleotide sequence.

11. The nucleic acid molecule of claim 5, wherein the nucleic acid molecule comprises a nucleotide sequence selected from the group consisting of
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that immunospecifically-binds to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule, thereby determining the presence or amount of the nucleic acid molecule in said sample.

20. A method of identifying an agent that binds to a polypeptide of claim 1, the method comprising:
(a) contacting said polypeptide with said agent; and
(b) determining whether said agent binds to said polypeptide.

21. A method for identifying an agent that modulates the expression or activity of the polypeptide of claim 1, the method comprising:
(a) providing a cell expressing said polypeptide;
(b) contacting the cell with said agent; and
(c) determining whether the agent modulates expression or activity of said polypeptide, whereby an alteration in expression or activity of said peptide indicates said agent modulates expression or activity of said polypeptide.

22. A method for modulating the activity of the polypeptide of claim 1, the method comprising contacting a cell sample expressing the polypeptide of said claim with a compound that binds to said polypeptide in an amount sufficient to modulate the activity of the polypeptide.

23. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

24. The method of claim 23, wherein said subject is a human.

25. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

26. The method of claim 25, wherein said subject is a human.

27. A method of treating or preventing a NOVX-associated disorder, said method comprising administering to a subject in which such treatment or prevention is desired the antibody of claim 15 in an amount sufficient to treat or prevent said NOVX-associated disorder in said subject.

28. The method of claim 27, wherein the subject is a human.

29. A pharmaceutical composition comprising the polypeptide of claim 1 and a pharmaceutically-acceptable carrier.

30. A pharmaceutical composition comprising the nucleic acid molecule of claim 5 and a pharmaceutically-acceptable carrier.

31. A pharmaceutical composition comprising the antibody of claim 15 and a pharmaceutically-acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. The use of a therapeutic in the manufacture of a medicament for treating a syndrome associated with a human disease, the disease selected from a NOVX-associated disorder, wherein said therapeutic is selected from the group consisting of a NOVX polypeptide, a NOVX nucleic acid, and a NOVX antibody.

36. A method for screening for a modulator of activity or of latency or predisposition to a NOVX-associated disorder, said method comprising:
(a) administering a test compound to a test animal at increased risk for a NOVX-associated disorder, wherein said test animal recombinantly expresses the polypeptide of claim 1;
(b) measuring the activity of said polypeptide in said test animal after administering the compound of step (a);
(c) comparing the activity of said protein in said test animal with the activity of said polypeptide in a control animal not administered said polypeptide, wherein a change in the activity of said polypeptide in said test animal relative to said control animal indicates the test compound is a modulator of latency of or predisposition to a NOVX-associated disorder.

37. The method of claim 36, wherein said test animal is a recombinant test animal that expresses a test protein transgene or expresses said transgene under the control of a promoter at an increased level relative to a wild-type test animal, and wherein said promoter is not the native gene promoter of said transgene.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

39. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

40. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54 and 56, or a biologically active fragment thereof.

41. A method of treating a pathological state in a mammal, the method comprising administering to the mammal the antibody of claim 15 in an amount sufficient to alleviate the pathological state.
